(19) United States
(12) Patent Application Publication
(10) Pub. No.: US 2025/0367309 A1
(43) Pub. Date: Dec. 4, 2025

(54) METHODS AND COMPOSITIONS FOR OLIGONUCLEOTIDE BIOCONJUGATION

(71) Applicant: Darbix LLC, Salt Lake City, UT (US)

(72) Inventors: Heemal Hansraj Dhanjee, Salem, MA (US); Bradley Lether Pentelute, Revere, MA (US); Jacob Joshua Lee Rodriguez, Brighton, MA (US); Daniel Jean-Francois Chinnapen, Quincy, MA (US)

(73) Assignee: Darbix LLC, Salt Lake City, UT (US)

(21) Appl. No.: 19/226,138

(22) Filed: Jun. 2, 2025

Related U.S. Application Data
(60) Provisional application No. 63/655,365, filed on Jun. 3, 2024.
(60) Provisional application No. 63/815,222, filed on May 30, 2025.

(51) Int. Cl. A61K 47/68; A61K 47/58; A61K 47/64; C07K 16/28; C12N 15/85

(52) U.S. Cl. CPC A61K 47/6807; A61K 47/58; A61K 47/64; A61K 47/6849; C07K 16/2818; C12N 15/85

(57) ABSTRACT

The present disclosure relates to oligonucleotide bioconjugates for targeted therapy, and processes to make the same. The present disclosure also relates to mRNA bioconjugates and pharmaceutical formulations thereof which prevent, slow the progression, or reduce the severity of cancer. Additionally, the present disclosure relates to mRNA bioconjugates and pharmaceutical formulations thereof which prevent, slow the progression, or reduce the severity of obesity or one or more other metabolic and/or cardiovascular disorders.

### CROSS REFERENCE TO RELATED APPLICATIONS

This application claims the benefit of U.S. Provisional Application No. 63/655,365, filed Jun. 3, 2024, and U.S. Provisional Application No. 63/815,222, filed May 30, 2025, each of which is herein incorporated by reference in its entirety.

### INCORPORATION OF SEQUENCE LISTING

A sequence listing contained in the file named “P35513_SL.xml” which is 281,944 bytes (measured in operating system MS-Windows®), created on Jun. 2, 2025, containing a total number of 129 sequences, starting from SEQ ID NO:1 to SEQ ID NO: 129, is filed electronically herewith and incorporated by reference in its entirety.

### FIELD OF THE INVENTION

The present disclosure relates to oligonucleotide bioconjugates and pharmaceutical formulations thereof for therapy, and processes to make the same. The present disclosure also relates to preventing, slowing the progression, or reducing the severity of cancer in a patient in need thereof by administering to the patient mRNA bioconjugates or pharmaceutical formulations thereof. The present disclosure also pertains to preventing, slowing the progression, or reducing the severity of obesity in a patient in need thereof by administering to the patient mRNA bioconjugates or pharmaceutical formulations thereof.

### BACKGROUND

The use of messenger RNA (mRNA) for therapeutic applications is more challenging compared to other biomolecules like DNA, polypeptides and proteins, due in large part to the many difficulties in cytosolic delivery. Like many proteins, mRNAs display poor pharmacokinetic properties in vivo, such as stimulation of the innate immune response, which results in their rapid degradation and renal clearance. RNA molecules are also susceptible to enzymatic degradation by RNAses in the bloodstream and tissues, rendering them deactivated before they arrive at their target locations. Once they arrive at their target cells, mRNAs have difficulty passing through cellular membranes in order to enter the cytoplasm of cells, due to their large size and polyanionic nature. Even after being internalized, exogenous RNA molecules tend to be sequestered within endosomal compartments. While DNA, polypeptides and proteins can be covalently modified with polymers, peptides, and other macromolecules to alleviate these problems, similar bioconjugation strategies for site-specific covalent modification of mRNA do not yet exist.

Circularization of mRNA can increase their stability by protecting the 3′-poly(A) tails from nonspecific 3′-exonucleases but does not allow for functional handles to be covalently added. Present bioconjugation strategies for site-specific covalent modification of mRNA run into several difficulties. It is difficult to add functional groups to internal bases due to the similar reactivity between any given purine or pyrimidine base, and the relatively low nucleophilicity of any ribose alcohol within an mRNA. On the other hand, the 5′ guanine cap of mRNAs is more easily modified by methyltransferase enzymes or the addition of photocleavable groups during synthesis, but the modified mRNA cap often arrests cap-dependent translation or yields a much lower translation efficiency compared to the unmodified mRNA.

The prior art provides generalized methods related to bioconjugation. For example, U.S. Pat. No. 10,925,935 (“the '935 Patent”) provides compositions and methods for the manufacture and modified mRNA molecules via optimization of their terminal architecture. International Patent Publication No. WO2023/212,213 A1 (“the '213 Publication”) provides tail-to-tail RNA conjugates translatable by eukaryotic ribosomes. International Patent Publication No. WO2023/250,528 A1 (“the '528 Publication”) provides compositions, reagents, and methods for producing capped, circular RNA molecules, circularized RNA molecules, and in particular, circularized mRNA molecules encoding a polypeptide such as a therapeutic protein. International Patent Publication No. WO2017/177,169 A1 (“the '169 Publication”) provides multimeric coding nucleic acids.

However, there still exists a need for new chemical techniques to modify and bioconjugate mRNA efficiently, to enable more effective drug delivery platforms.

### SUMMARY

The present specification addresses the need to identify compositions and methods for targeted therapy in a patient in need thereof using oligonucleotide bioconjugates or mRNA bioconjugates. The present specification also addresses the need to identify oligonucleotide bioconjugate or mRNA bioconjugate compositions and methods for treating obesity cancer in a patient in need thereof. The present specification also addresses the need to identify oligonucleotide bioconjugate or mRNA bioconjugate compositions and methods for treating obesity in a patient in need thereof. The present specification also addresses the need for robust methods for the preparation of oligonucleotide bioconjugates and mRNA bioconjugates.

In an aspect, the present specification provides, and includes, an oligonucleotide bioconjugate, or a pharmaceutically acceptable salt thereof, of formula (I)

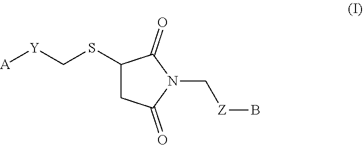

wherein
   A is an oligonucleotide having a 3′-end and a 5′-end;
   Y is a C₁-C₂₀ alkyl, wherein any one or more —CH₂— groups of the C₁-C₂₀ alkyl is each optionally replaced independently with —C(═O)—, —CF₂—, or a heteroatomic moiety selected from the group consisting of —O—, —S—, —NH—, and —N(C₁-C₆ alkyl)-, wherein any one or more —CH₃ groups of the C₁-C₂₀ alkyl is each optionally replaced independently with —CF₃, —CF₂H, —CH₂F, or a heteroatomic moiety selected from the group consisting of —NH(C₁-C₆ alkyl), —N(C₁-C₆ alkyl)₂, —O(C₁-C₆ alkyl), and —S(C₁-C₆ alkyl), and wherein any two heteroatomic moieties are separated from one another by at least two carbon atoms;
   Z is a C₁-C₂₀ alkyl, wherein any one or more —CH₂— groups of the C₁-C₂₀ alkyl is each optionally replaced independently with —C(═O)—, —CF₂—, or a heteroatomic moiety selected from the group consisting of —O—, —S—, —NH—, and —N(C₁-C₆ alkyl)-, wherein any one or more —CH₃ groups of the C₁-C₂₀ alkyl is each optionally replaced independently with —CF₃, —CF₂H, —CH₂F, or a heteroatomic moiety selected from the group consisting of —NH(C₁-C₆ alkyl), —N(C₁-C₆ alkyl)₂, —O(C₁-C₆ alkyl), and —S(C₁-C₆ alkyl), and wherein any two heteroatomic moieties are separated from one another by at least two carbon atoms; and
   B is an oligonucleotide, a polypeptide, a protein, a small molecule, a carbohydrate, a lipid, a polyethylene glycol (PEG) molecule, or a biopolymer,
wherein Y is covalently attached to the 3′-end of A through a first linkage.

In an aspect, the present specification provides, and includes, an oligonucleotide bioconjugate, or a pharmaceutically acceptable salt thereof, of formula (II)

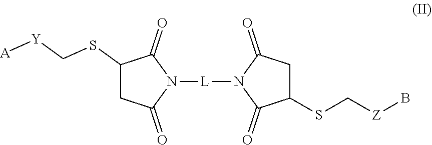

wherein
   A is an oligonucleotide having a 3′-end and a 5′-end;
   Y is a C₁-C₂₀ alkyl, wherein any one or more —CH₂— groups of the C₁-C₂₀ alkyl is each optionally replaced independently with —C(═O)—, —CF₂—, or a heteroatomic moiety selected from the group consisting of —O—, —S—, —NH—, and —N(C₁-C₆ alkyl)-, wherein any one or more —CH₃ groups of the C₁-C₂₀ alkyl is each optionally replaced independently with —CF₃, —CF₂H, —CH₂F, or a heteroatomic moiety selected from the group consisting of —NH(C₁-C₆ alkyl), —N(C₁-C₆ alkyl)₂, —O(C₁-C₆ alkyl), and —S(C₁-C₆ alkyl), and wherein any two heteroatomic moieties are separated from one another by at least two carbon atoms;
   L is a C₂-C₅₀ alkyl, C₂-C₅₀ alkenyl, C₂-C₅₀ alkynyl, C₃-C₈ cycloalkyl, aryl, heteroaryl, C₄-C₅₀ alkyl-cycloalkyl, C₇-C₅₀ alkyl-aryl, or C₆-C₅₀ alkyl-heteroaryl, wherein any one or more —CH₂— groups of L is each optionally replaced independently with —C(═O)—, —CF₂—, or a heteroatomic moiety selected from the group consisting of —O—, —S—, —NH—, and —N(C₁-C₆ alkyl)-, wherein any one or more —CH₃ groups of L is each optionally replaced independently with —CF₃, —CF₂H, —CH₂F, or a heteroatomic moiety selected from the group consisting of —NH(C₁-C₆ alkyl), —N(C₁-C₆alkyl)₂, —O(C₁-C₆ alkyl), and —S(C₁-C₆ alkyl), and wherein any two heteroatomic moieties are separated from one another by at least two carbon atoms;
   Z is a C₁-C₂₀ alkyl, wherein any one or more —CH₂— groups of the C₁-C₂₀ alkyl is each optionally replaced independently with —C(═O)—, —CF₂—, or a heteroatomic moiety selected from the group consisting of —O—, —S—, —NH—, and —N(C₁-C₆ alkyl)-, wherein any one or more —CH₃ groups of the C₁-C₂₀ alkyl is each optionally replaced independently with —CF₃, —CF₂H, —CH₂F, or a heteroatomic moiety selected from the group consisting of —NH(C₁-C₆ alkyl), —N(C₁-C₆ alkyl)₂, —O(C₁-C₆ alkyl), and —S(C₁-C₆ alkyl), and wherein any two heteroatomic moieties are separated from one another by at least two carbon atoms; and
   B is an oligonucleotide, a polypeptide, a protein, a small molecule, a lipid, a carbohydrate, a polyethylene glycol (PEG) molecule, or a biopolymer, wherein Y is covalently attached to the 3′-end of A through a first linkage.

In an aspect, the present specification provides, and includes, an oligonucleotide bioconjugate, or a pharmaceutically acceptable salt thereof, of formula (III)

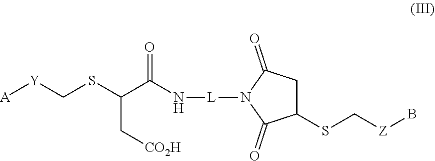

wherein
   A is an oligonucleotide having a 3′-end and a 5′-end;
   Y is a C₁-C₂₀ alkyl, wherein any one or more —CH₂— groups of the C₁-C₂₀ alkyl is each optionally replaced independently with —C(═O)—, —CF₂—, or a heteroatomic moiety selected from the group consisting of —O—, —S—, —NH—, and —N(C₁-C₆ alkyl)-, wherein any one or more —CH₃ groups of the C₁-C₂₀ alkyl is each optionally replaced independently with —CF₃, —CF₂H, —CH₂F, or a heteroatomic moiety selected from the group consisting of —NH(C₁-C₆ alkyl), —N(C₁-C₆ alkyl)₂, —O(C₁-C₆ alkyl), and —S(C₁-C₆ alkyl), and wherein any two heteroatomic moieties are separated from one another by at least two carbon atoms;
   L is a C₂-C₅₀ alkyl, C₂-C₅₀ alkenyl, C₂-C₅₀ alkynyl, C₃-C₈ cycloalkyl, aryl, heteroaryl, C₄-C₅₀ alkyl-cycloalkyl, C₇-C₅₀ alkyl-aryl, or C₆-C₅₀ alkyl-heteroaryl, wherein any one or more —CH₂— groups of L is each optionally replaced independently with —C(═O)—, —CF₂—, or a heteroatomic moiety selected from the group consisting of —O—, —S—, —NH—, and —N(C₁-C₆ alkyl)-, wherein any one or more —CH₃ groups of L is each optionally replaced independently with —CF₃, —CF₂H, —CH₂F, or a heteroatomic moiety selected from the group consisting of —NH(C₁-C₆ alkyl), —N(C₁-C₆alkyl)₂, —O(C₁-C₆ alkyl), and —S(C₁-C₆ alkyl), and wherein any two heteroatomic moieties are separated from one another by at least two carbon atoms;
   Z is a C₁-C₂₀ alkyl, wherein any one or more —CH₂— groups of the C₁-C₂₀ alkyl is each optionally replaced independently with —C(═O)—, —CF₂—, or a heteroatomic moiety selected from the group consisting of —O—, —S—, —NH—, and —N(C₁-C₆ alkyl)-, wherein any one or more —CH₃ groups of the C₁-C₂₀ alkyl is each optionally replaced independently with —CF₃, —CF₂H, —CH₂F, or a heteroatomic moiety selected from the group consisting of —NH(C₁-C₆ alkyl), —N(C₁-C₆ alkyl)₂, —O(C₁-C₆ alkyl), and —S(C₁-C₆ alkyl), and wherein any two heteroatomic moieties are separated from one another by at least two carbon atoms; and
   B is an oligonucleotide, a polypeptide, a protein, a small molecule, a lipid, a carbohydrate, a polyethylene glycol (PEG) molecule, or a biopolymer,
wherein Y is covalently attached to the 3′-end of A through a first linkage.

In an aspect, the present specification provides, and includes, an oligonucleotide bioconjugate, or a pharmaceutically acceptable salt thereof, of formula (IV)

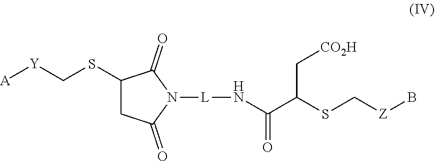

wherein
   A is an oligonucleotide having a 3′-end and a 5′-end;
   Y is a C₁-C₂₀ alkyl, wherein any one or more —CH₂— groups of the C₁-C₂₀ alkyl is each optionally replaced independently with —C(═O)—, —CF₂—, or a heteroatomic moiety selected from the group consisting of —O—, —S—, —NH—, and —N(C₁-C₆ alkyl)-, wherein any one or more —CH₃ groups of the C₁-C₂₀ alkyl is each optionally replaced independently with —CF₃, —CF₂H, —CH₂F, or a heteroatomic moiety selected from the group consisting of —NH(C₁-C₆ alkyl), —N(C₁-C₆ alkyl)₂, —O(C₁-C₆ alkyl), and —S(C₁-C₆ alkyl), and wherein any two heteroatomic moieties are separated from one another by at least two carbon atoms;
   L is a C₂-C₅₀ alkyl, C₂-C₅₀ alkenyl, C₂-C₅₀ alkynyl, C₃-C₈ cycloalkyl, aryl, heteroaryl, C₄-C₅₀ alkyl-cycloalkyl, C₇-C₅₀ alkyl-aryl, or C₆-C₅₀ alkyl-heteroaryl, wherein any one or more —CH₂— groups of L is each optionally replaced independently with —C(═O)—, —CF₂—, or a heteroatomic moiety selected from the group consisting of —O—, —S—, —NH—, and —N(C₁-C₆ alkyl)-, wherein any one or more —CH₃ groups of L is each optionally replaced independently with —CF₃, —CF₂H, —CH₂F, or a heteroatomic moiety selected from the group consisting of —NH(C₁-C₆ alkyl), —N(C₁-C₆alkyl)₂, —O(C₁-C₆ alkyl), and —S(C₁-C₆ alkyl), and wherein any two heteroatomic moieties are separated from one another by at least two carbon atoms;
   Z is a C₁-C₂₀ alkyl, wherein any one or more —CH₂— groups of the C₁-C₂₀ alkyl is each optionally replaced independently with —C(═O)—, —CF₂—, or a heteroatomic moiety selected from the group consisting of —O—, —S—, —NH—, and —N(C₁-C₆ alkyl)-, wherein any one or more —CH₃ groups of the C₁-C₂₀ alkyl is each optionally replaced independently with —CF₃, —CF₂H, —CH₂F, or a heteroatomic moiety selected from the group consisting of —NH(C₁-C₆ alkyl), —N(C₁-C₆ alkyl)₂, —O(C₁-C₆ alkyl), and —S(C₁-C₆ alkyl), and wherein any two heteroatomic moieties are separated from one another by at least two carbon atoms; and
   B is an oligonucleotide, a polypeptide, a protein, a small molecule, a lipid, a carbohydrate, a polyethylene glycol (PEG) molecule, or a biopolymer,
wherein Y is covalently attached to the 3′-end of A through a first linkage.

In an aspect, the present specification provides, and includes, an oligonucleotide bioconjugate, or a pharmaceutically acceptable salt thereof, of formula (V)

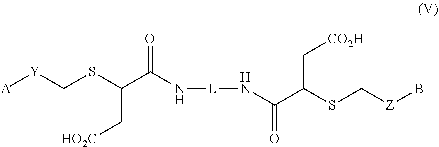

wherein
   A is an oligonucleotide having a 3′-end and a 5′-end;
   Y is a C₁-C₂₀ alkyl, wherein any one or more —CH₂— groups of the C₁-C₂₀ alkyl is each optionally replaced independently with —C(═O)—, —CF₂—, or a heteroatomic moiety selected from the group consisting of —O—, —S—, —NH—, and —N(C₁-C₆ alkyl)-, wherein any one or more —CH₃ groups of the C₁-C₂₀ alkyl is each optionally replaced independently with —CF₃, —CF₂H, —CH₂F, or a heteroatomic moiety selected from the group consisting of —NH(C₁-C₆ alkyl), —N(C₁-C₆ alkyl)₂, —O(C₁-C₆ alkyl), and —S(C₁-C₆ alkyl), and wherein any two heteroatomic moieties are separated from one another by at least two carbon atoms;
   L is a C₂-C₅₀ alkyl, C₂-C₅₀ alkenyl, C₂-C₅₀ alkynyl, C₃-C₈ cycloalkyl, aryl, heteroaryl, C₄-C₅₀ alkyl-cycloalkyl, C₇-C₅₀ alkyl-aryl, or C₆-C₅₀ alkyl-heteroaryl, wherein any one or more —CH₂— groups of the L is each optionally replaced independently with —C(═O)—, —CF₂—, or a heteroatomic moiety selected from the group consisting of —O—, —S—, —NH—, and —N(C₁-C₆ alkyl)-, wherein any one or more —CH₃ groups of L is each optionally replaced independently with —CF₃, —CF₂H, —CH₂F, or a heteroatomic moiety selected from the group consisting of —NH(C₁-C₆ alkyl), —N(C₁-C₆alkyl)₂, —O(C₁-C₆ alkyl), and —S(C₁-C₆ alkyl), and wherein any two heteroatomic moieties are separated from one another by at least two carbon atoms;
   Z is a C₁-C₂₀ alkyl, wherein any one or more —CH₂— groups of the C₁-C₂₀ alkyl is each optionally replaced independently with —C(═O)—, —CF₂—, or a heteroatomic moiety selected from the group consisting of —O—, —S—, —NH—, and —N(C₁-C₆ alkyl)-, wherein any one or more —CH₃ groups of the C₁-C₂₀ alkyl is each optionally replaced independently with —CF₃, —CF₂H, —CH₂F, or a heteroatomic moiety selected from the group consisting of —NH(C₁-C₆ alkyl), —N(C₁-C₆ alkyl)₂, —O(C₁-C₆ alkyl), and —S(C₁-C₆ alkyl), and wherein any two heteroatomic moieties are separated from one another by at least two carbon atoms; and
   B is an oligonucleotide, a polypeptide, a protein, a small molecule, a lipid, a carbohydrate, a polyethylene glycol (PEG) molecule, or a biopolymer,
wherein Y is covalently attached to the 3′-end of A through a first linkage.

In an aspect, the present specification provides, and includes a method of making an oligonucleotide bioconjugate, or a pharmaceutically acceptable salt thereof, of formula (I)

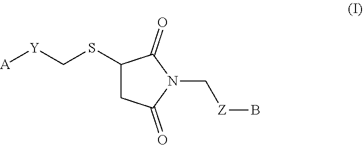

wherein
   A is an oligonucleotide having a 3′-end and a 5′-end;
   Y is a C₁-C₂₀ alkyl, wherein any one or more —CH₂— groups of the C₁-C₂₀ alkyl is each optionally replaced independently with —C(═O)—, —CF₂—, or a heteroatomic moiety selected from the group consisting of —O—, —S—, —NH—, and —N(C₁-C₆ alkyl)-, wherein any one or more —CH₃ groups of the C₁-C₂₀ alkyl is each optionally replaced independently with —CF₃, —CF₂H, —CH₂F, or a heteroatomic moiety selected from the group consisting of —NH(C₁-C₆ alkyl), —N(C₁-C₆ alkyl)₂, —O(C₁-C₆ alkyl), and —S(C₁-C₆ alkyl), and wherein any two heteroatomic moieties are separated from one another by at least two carbon atoms;
   L is a C₂-C₅₀ alkyl, C₂-C₅₀ alkenyl, C₂-C₅₀ alkynyl, C₃-C₅ cycloalkyl, aryl, heteroaryl, C₄-C₅₀ alkyl-cycloalkyl, C₇-C₅₀ alkyl-aryl, or C₆-C₅₀ alkyl-heteroaryl, wherein any one or more —CH₂— groups of the L is each optionally replaced independently with —C(═O)—, —CF₂—, or a heteroatomic moiety selected from the group consisting of —O—, —S—, —NH—, and —N(C₁-C₆ alkyl)-, wherein any one or more —CH₃ groups of L is each optionally replaced independently with —CF₃, —CF₂H, —CH₂F, or a heteroatomic moiety selected from the group consisting of —NH(C₁-C₆ alkyl), —N(C₁-C₆ alkyl)₂, —O(C₁-C₆ alkyl), and —S(C₁-C₆ alkyl), and wherein any two heteroatomic moieties are separated from one another by at least two carbon atoms;
   Z is a C₁-C₂₀ alkyl, wherein any one or more —CH₂— groups of the C₁-C₂₀ alkyl is each optionally replaced independently with —C(═O)—, —CF₂—, or a heteroatomic moiety selected from the group consisting of —O—, —S—, —NH—, and —N(C₁-C₆ alkyl)-, wherein any one or more —CH₃ groups of the C₁-C₂₀ alkyl is each optionally replaced independently with —CF₃, —CF₂H, —CH₂F, or a heteroatomic moiety selected from the group consisting of —NH(C₁-C₆ alkyl), —N(C₁-C₆ alkyl)₂, —O(C₁-C₆ alkyl), and —S(C₁-C₆ alkyl), and wherein any two heteroatomic moieties are separated from one another by at least two carbon atoms; and
   B is an oligonucleotide, a polypeptide, a protein, a small molecule, a lipid, a carbohydrate, a polyethylene glycol (PEG) molecule, or a biopolymer,
wherein Y is covalently attached to the 3′-end of A through a first linkage, the method comprising a step of reacting the thiol of formula (VI) with the maleimide of formula (VII) to form the oligonucleotide bioconjugate of formula (I)

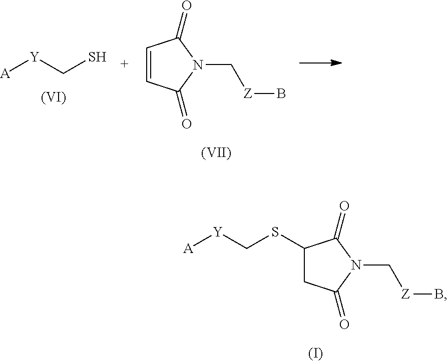

wherein A and Y of formula (VI) are defined as above for formula (I), and wherein Z and B of formula (VII) are defined as above for formula (I).

In an aspect, the present specification provides, and includes a method of making an oligonucleotide bioconjugate, or a pharmaceutically acceptable salt thereof, of formula (II)

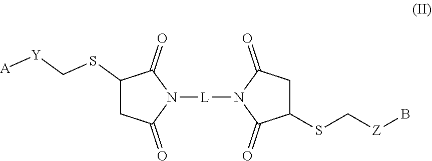

wherein
   A is an oligonucleotide having a 3′-end and a 5′-end;
   Y is a C₁-C₂₀ alkyl, wherein any one or more —CH₂— groups of the C₁-C₂₀ alkyl is each optionally replaced independently with —C(═O)—, —CF₂—, or a heteroatomic moiety selected from the group consisting of —O—, —S—, —NH—, and —N(C₁-C₆ alkyl)-, wherein any one or more —CH₃ groups of the C₁-C₂₀ alkyl is each optionally replaced independently with —CF₃, —CF₂H, —CH₂F, or a heteroatomic moiety selected from the group consisting of —NH(C₁-C₆ alkyl), —N(C₁-C₆ alkyl)₂, —O(C₁-C₆ alkyl), and —S(C₁-C₆ alkyl), and wherein any two heteroatomic moieties are separated from one another by at least two carbon atoms;
   L is a C₂-C₅₀ alkyl, C₂-C₅₀ alkenyl, C₂-C₅₀ alkynyl, C₃-C₈ cycloalkyl, aryl, heteroaryl, C₄-C₅₀ alkyl-cycloalkyl, C₇-C₅₀ alkyl-aryl, or C₆-C₅₀ alkyl-heteroaryl, wherein any one or more —CH₂— groups of L is each optionally replaced independently with —C(═O)—, —CF₂—, or a heteroatomic moiety selected from the group consisting of —O—, —S—, —NH—, and —N(C₁-C₆ alkyl)-, wherein any one or more —CH₃ groups of L is each optionally replaced independently with —CF₃, —CF₂H, —CH₂F, or a heteroatomic moiety selected from the group consisting of —NH(C₁-C₆ alkyl), —N(C₁-C₆alkyl)₂, —O(C₁-C₆ alkyl), and —S(C₁-C₆ alkyl), and wherein any two heteroatomic moieties are separated from one another by at least two carbon atoms;
   Z is a C₁-C₂₀ alkyl, wherein any one or more —CH₂— groups of the C₁-C₂₀ alkyl is each optionally replaced independently with —C(═O)—, —CF₂—, or a heteroatomic moiety selected from the group consisting of —O—, —S—, —NH—, and —N(C₁-C₆ alkyl)-, wherein any one or more —CH₃ groups of the C₁-C₂₀ alkyl is each optionally replaced independently with —CF₃, —CF₂H, —CH₂F, or a heteroatomic moiety selected from the group consisting of —NH(C₁-C₆ alkyl), —N(C₁-C₆ alkyl)₂, —O(C₁-C₆ alkyl), and —S(C₁-C₆ alkyl), and wherein any two heteroatomic moieties are separated from one another by at least two carbon atoms; and
   B is an oligonucleotide, a polypeptide, a protein, a small molecule, a lipid, a carbohydrate, a polyethylene glycol (PEG) molecule, or a biopolymer,
   wherein Y is covalently attached to the 3′-end of A through a first linkage,
the method comprising the steps of
(i) reacting the thiol of formula (VI) with one of the two maleimides of formula (VIII) to form a compound of formula (IX)

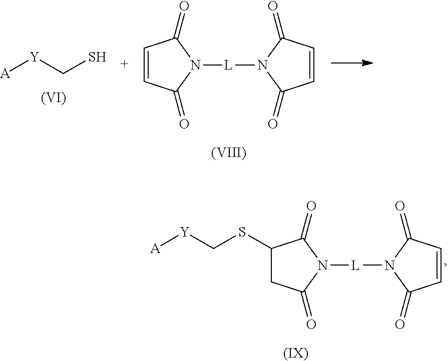

wherein A and Y of formula (VI) are defined as above for formula (II), and wherein L is defined as above for formula (II), and
(ii) reacting the maleimide of formula (IX) with the thiol of formula (X) to form the oligonucleotide bioconjugate of formula (II)

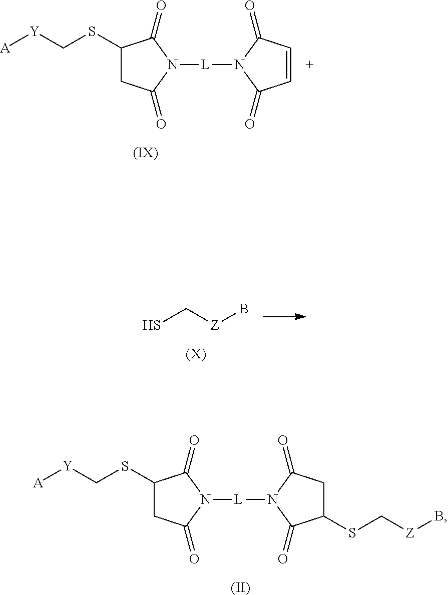

wherein Z and B of formula (X) are as defined above for formula (II).

In an aspect, the present specification provides, and includes a method of making an oligonucleotide bioconjugate, or a pharmaceutically acceptable salt thereof, of formula (III)

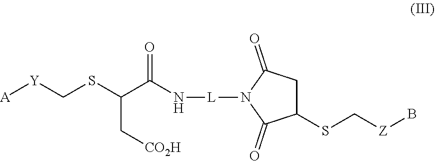

wherein
   A is an oligonucleotide having a 3′-end and a 5′-end;
   Y is a C₁-C₂₀ alkyl, wherein any one or more —CH₂— groups of the C₁-C₂₀ alkyl is each optionally replaced independently with —C(═O)—, —CF₂—, or a heteroatomic moiety selected from the group consisting of —O—, —S—, —NH—, and —N(C₁-C₆ alkyl)-, wherein any one or more —CH₃ groups of the C₁-C₂₀ alkyl is each optionally replaced independently with —CF₃, —CF₂H, —CH₂F, or a heteroatomic moiety selected from the group consisting of —NH(C₁-C₆ alkyl), —N(C₁-C₆ alkyl)₂, —O(C₁-C₆ alkyl), and —S(C₁-C₆ alkyl), and wherein any two heteroatomic moieties are separated from one another by at least two carbon atoms;
   L is a C₂-C₅₀ alkyl, C₂-C₅₀ alkenyl, C₂-C₅₀ alkynyl, C₃-C₅ cycloalkyl, aryl, heteroaryl, C₄-C₅₀ alkyl-cycloalkyl, C₇-C₅₀ alkyl-aryl, or C₆-C₅₀ alkyl-heteroaryl, wherein any one or more —CH₂— groups of L is each optionally replaced independently with —C(═O)—, —CF₂—, or a heteroatomic moiety selected from the group consisting of —O—, —S—, —NH—, and —N(C₁-C₆ alkyl)-, wherein any one or more —CH₃ groups of L is each optionally replaced independently with —CF₃, —CF₂H, —CH₂F, or a heteroatomic moiety selected from the group consisting of —NH(C₁-C₆ alkyl), —N(C₁-C₆ alkyl)₂, —O(C₁-C₆ alkyl), and —S(C₁-C₆ alkyl), and wherein any two heteroatomic moieties are separated from one another by at least two carbon atoms;
   Z is a C₁-C₂₀ alkyl, wherein any one or more —CH₂— groups of the C₁-C₂₀ alkyl is each optionally replaced independently with —C(═O)—, —CF₂—, or a heteroatomic moiety selected from the group consisting of —O—, —S—, —NH—, and —N(C₁-C₆ alkyl)-, wherein any one or more —CH₃ groups of the C₁-C₂₀ alkyl is each optionally replaced independently with —CF₃, —CF₂H, —CH₂F, or a heteroatomic moiety selected from the group consisting of —NH(C₁-C₆ alkyl), —N(C₁-C₆ alkyl)₂, —O(C₁-C₆ alkyl), and —S(C₁-C₆ alkyl), and wherein any two heteroatomic moieties are separated from one another by at least two carbon atoms; and
   B is an oligonucleotide, a polypeptide, a protein, a small molecule, a lipid, a carbohydrate, a polyethylene glycol (PEG) molecule, or a biopolymer,
wherein Y is covalently attached to the 3′-end of A through a first linkage,
the method comprising the steps of
(i) reacting the thiol of formula (VI) with one of the two maleimides of formula (VIII) to form a compound of formula (IX)

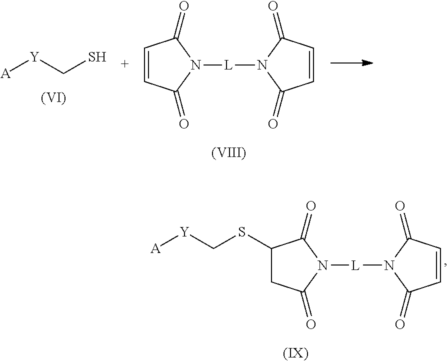

wherein A and Y of formula (VI) are defined as above for formula (III), and wherein L of formula (VIII) is defined as above for formula (III),
(ii) reacting the maleimide of formula (IX) with the thiol of formula (X) to form a compound of formula (II)

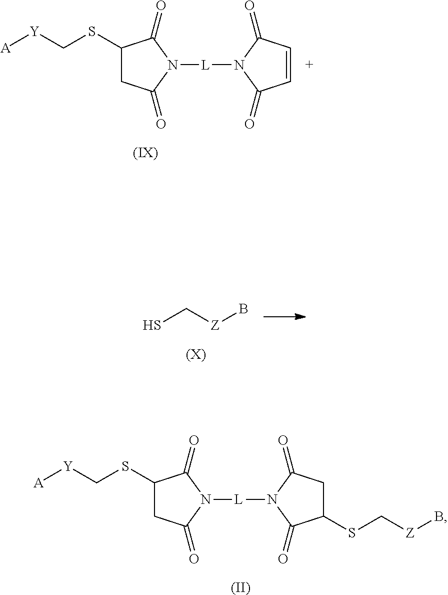

wherein Z and B of formula (X) are as defined above for formula (III), and
(iii) hydrolyzing one of the succinimides of formula (II) to form the oligonucleotide bioconjugate of formula (III)

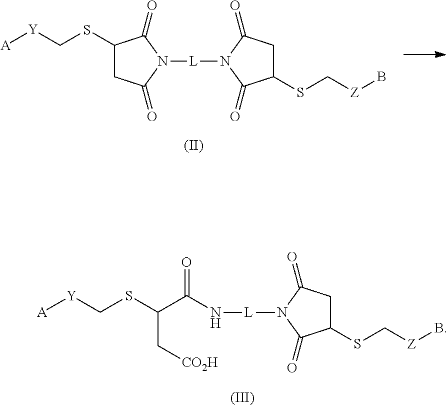

In an aspect, the present specification provides, and includes a method of making an oligonucleotide bioconjugate, or a pharmaceutically acceptable salt thereof, of formula (IV)

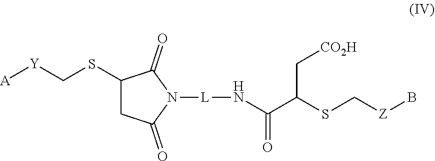

wherein
   A is an oligonucleotide having a 3′-end and a 5′-end;
   Y is a C₁-C₂₀ alkyl, wherein any one or more —CH₂— groups of the C₁-C₂₀ alkyl is each optionally replaced independently with —C(═O)—, —CF₂—, or a heteroatomic moiety selected from the group consisting of —O—, —S—, —NH—, and —N(C₁-C₆ alkyl)-, wherein any one or more —CH₃ groups of the C₁-C₂₀ alkyl is each optionally replaced independently with —CF₃, —CF₂H, —CH₂F, or a heteroatomic moiety selected from the group consisting of —NH(C₁-C₆ alkyl), —N(C₁-C₆ alkyl)₂, —O(C₁-C₆ alkyl), and —S(C₁-C₆ alkyl), and wherein any two heteroatomic moieties are separated from one another by at least two carbon atoms;
   L is a C₂-C₅₀ alkyl, C₂-C₅₀ alkenyl, C₂-C₅₀ alkynyl, C₃-C₈ cycloalkyl, aryl, heteroaryl, C₄-C₅₀ alkyl-cycloalkyl, C₇-C₅₀ alkyl-aryl, or C₆-C₅₀ alkyl-heteroaryl, wherein any one or more —CH₂— groups of L is each optionally replaced independently with —C(═O)—, —CF₂—, or a heteroatomic moiety selected from the group consisting of —O—, —S—, —NH—, and —N(C₁-C₆ alkyl)-, wherein any one or more —CH₃ groups of L is each optionally replaced independently with —CF₃, —CF₂H, —CH₂F, or a heteroatomic moiety selected from the group consisting of —NH(C₁-C₆ alkyl), —N(C₁-C₆ alkyl)₂, —O(C₁-C₆ alkyl), and —S(C₁-C₆ alkyl), and wherein any two heteroatomic moieties are separated from one another by at least two carbon atoms;
   Z is a C₁-C₂₀ alkyl, wherein any one or more —CH₂— groups of the C₁-C₂₀ alkyl is each optionally replaced independently with —C(═O)—, —CF₂—, or a heteroatomic moiety selected from the group consisting of —O—, —S—, —NH—, and —N(C₁-C₆ alkyl)-, wherein any one or more —CH₃ groups of the C₁-C₂₀ alkyl is each optionally replaced independently with —CF₃, —CF₂H, —CH₂F, or a heteroatomic moiety selected from the group consisting of —NH(C₁-C₆ alkyl), —N(C₁-C₆ alkyl)₂, —O(C₁-C₆ alkyl), and —S(C₁-C₆ alkyl), and wherein any two heteroatomic moieties are separated from one another by at least two carbon atoms; and
   B is an oligonucleotide, a polypeptide, a protein, a small molecule, a lipid, a carbohydrate, a polyethylene glycol (PEG) molecule, or a biopolymer,
wherein Y is covalently attached to the 3′-end of A through a first linkage,
the method comprising the steps of
(i) reacting the thiol of formula (VI) with the maleimide of formula (VIII) to form a compound of formula (IX)

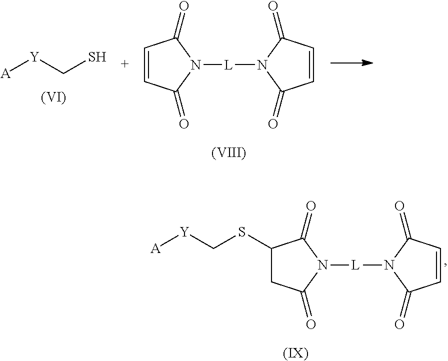

wherein A and Y of formula (VI) are defined as above for formula (IV), and wherein L of formula (VIII) is defined as above for formula (IV),
(ii) reacting the maleimide of formula (IX) with the thiol of formula (X) to form a compound of formula (II)

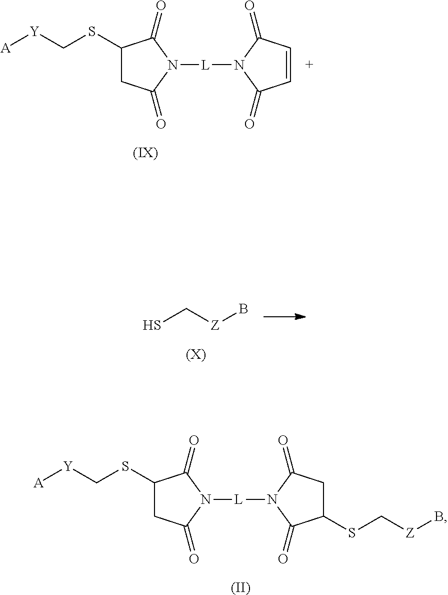

wherein Z and B of formula (X) are as defined above for formula (IV), and
(iii) hydrolyzing one of the succinimides of formula (II) to form the oligonucleotide bioconjugate of formula (IV)

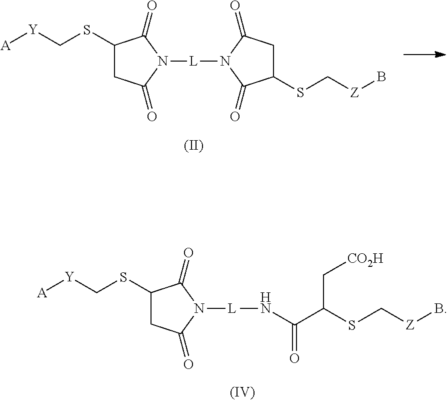

In an aspect, the present specification provides, and includes a method of making an oligonucleotide bioconjugate, or a pharmaceutically acceptable salt thereof, of formula (V)

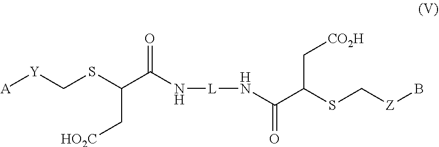

wherein
   A is an oligonucleotide having a 3′-end and a 5′-end;
   Y is a C₁-C₂₀ alkyl, wherein any one or more —CH₂— groups of the C₁-C₂₀ alkyl is each optionally replaced independently with —C(═O)—, —CF₂—, or a heteroatomic moiety selected from the group consisting of —O—, —S—, —NH—, and —N(C₁-C₆ alkyl)-, wherein any one or more —CH₃ groups of the C₁-C₂₀ alkyl is each optionally replaced independently with —CF₃, —CF₂H, —CH₂F, or a heteroatomic moiety selected from the group consisting of —NH(C₁-C₆ alkyl), —N(C₁-C₆ alkyl)₂, —O(C₁-C₆ alkyl), and —S(C₁-C₆ alkyl), and wherein any two heteroatomic moieties are separated from one another by at least two carbon atoms;
   L is a C₂-C₅₀ alkyl, C₂-C₅₀ alkenyl, C₂-C₅₀ alkynyl, C₃-C₅ cycloalkyl, aryl, heteroaryl, C₄-C₅₀ alkyl-cycloalkyl, C₇-C₅₀ alkyl-aryl, or C₆-C₅₀ alkyl-heteroaryl, wherein any one or more —CH₂— groups of L is each optionally replaced independently with —C(═O)—, —CF₂—, or a heteroatomic moiety selected from the group consisting of —O—, —S—, —NH—, and —N(C₁-C₆ alkyl)-, wherein any one or more —CH₃ groups of L is each optionally replaced independently with —CF₃, —CF₂H, —CH₂F, or a heteroatomic moiety selected from the group consisting of —NH(C₁-C₆ alkyl), —N(C₁—C₆ alkyl)₂, —O(C₁-C₆ alkyl), and —S(C₁-C₆ alkyl), and wherein any two heteroatomic moieties are separated from one another by at least two carbon atoms;
   Z is a C₁-C₂₀ alkyl, wherein any one or more —CH₂— groups of the C₁-C₂₀ alkyl is each optionally replaced independently with —C(═O)—, —CF₂—, or a heteroatomic moiety selected from the group consisting of —O—, —S—, —NH—, and —N(C₁-C₆ alkyl)-, wherein any one or more —CH₃ groups of the C₁-C₂₀ alkyl is each optionally replaced independently with —CF₃, —CF₂H, —CH₂F, or a heteroatomic moiety selected from the group consisting of —NH(C₁-C₆ alkyl), —N(C₁-C₆ alkyl)₂, —O(C₁-C₆ alkyl), and —S(C₁-C₆ alkyl), and wherein any two heteroatomic moieties are separated from one another by at least two carbon atoms; and
   B is an oligonucleotide, a polypeptide, a protein, a small molecule, a lipid, a carbohydrate, a polyethylene glycol (PEG) molecule, or a biopolymer,
wherein Y is covalently attached to the 3′-end of A through a first linkage,
the method comprising the steps of
(i) reacting the thiol of formula (VI) with one of the two maleimides of formula (VIII) to form a compound of formula (IX)

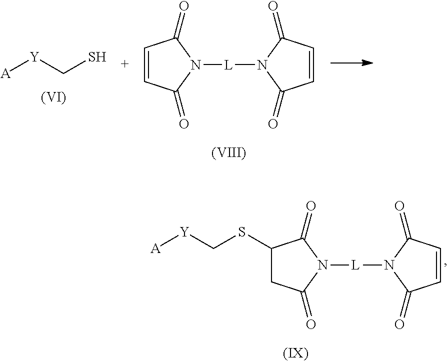

wherein A and Y of formula (VI) are defined as above for formula (V), and wherein L of formula (VIII) is defined as above for formula (V),
(ii) reacting the maleimide of formula (IX) with the thiol of formula (X) to form a compound of formula (II)

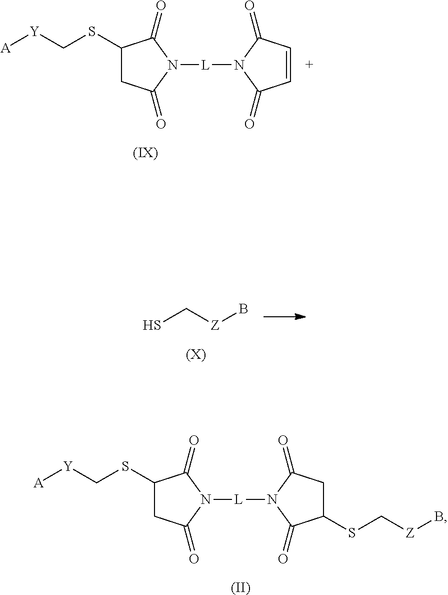

wherein Z and B of formula (X) are as defined above for formula (V), and (iii) hydrolyzing both of the succinimides of formula (II) to form the oligonucleotide bioconjugate of formula (V)

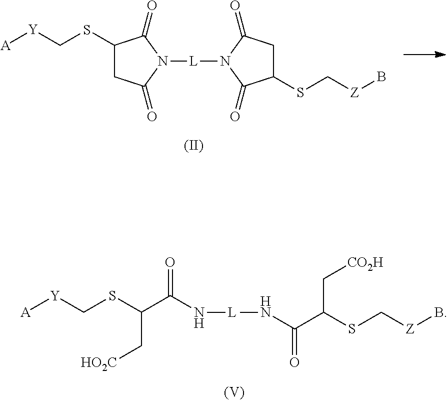

In an aspect, the present specification provides, and includes a method of co-expressing a first polypeptide and a second polypeptide in a cell, the method comprising a step of contacting the cell with an mRNA bioconjugate, or a pharmaceutically acceptable salt thereof, of formula (I)

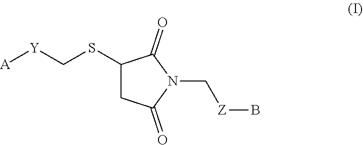

wherein
   A is a first mRNA molecule having a 3′-end and a 5′-end, wherein A encodes the first polypeptide;
   Y is a C₁-C₂₀ alkyl, wherein any one or more —CH₂— groups of the C₁-C₂₀ alkyl is each optionally replaced independently with —C(═O)—, —CF₂—, or a heteroatomic moiety selected from the group consisting of —O—, —S—, —NH—, and —N(C₁-C₆ alkyl)-, wherein any one or more —CH₃ groups of the C₁-C₂₀ alkyl is each optionally replaced independently with —CF₃, —CF₂H, —CH₂F, or a heteroatomic moiety selected from the group consisting of —NH(C₁-C₆ alkyl), —N(C₁-C₆ alkyl)₂, —O(C₁-C₆ alkyl), and —S(C₁-C₆ alkyl), and wherein any two heteroatomic moieties are separated from one another by at least two carbon atoms;
   Z is a C₁-C₂₀ alkyl, wherein any one or more —CH₂— groups of the C₁-C₂₀ alkyl is each optionally replaced independently with —C(═O)—, —CF₂—, or a heteroatomic moiety selected from the group consisting of —O—, —S—, —NH—, and —N(C₁-C₆ alkyl)-, wherein any one or more —CH₃ groups of the C₁-C₂₀ alkyl is each optionally replaced independently with —CF₃, —CF₂H, —CH₂F, or a heteroatomic moiety selected from the group consisting of —NH(C₁-C₆ alkyl), —N(C₁-C₆ alkyl)₂, —O(C₁-C₆ alkyl), and —S(C₁-C₆ alkyl), and wherein any two heteroatomic moieties are separated from one another by at least two carbon atoms; and
   B is a second mRNA molecule having a 3′-end and a 5′-end, wherein B encodes the second polypeptide,
wherein Y is covalently attached to the 3′-end of A through a first linkage, and wherein Z is covalently attached to the 3′-end of B through a second linkage.

In an aspect, the present specification provides, and includes a method of co-expressing a first polypeptide and a second polypeptide in a cell, the method comprising a step of contacting the cell with an mRNA bioconjugate, or a pharmaceutically acceptable salt thereof, of formula (II)

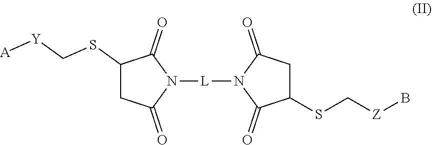

wherein
   A is a first mRNA molecule having a 3′-end and a 5′-end, wherein A encodes the first polypeptide;
   Y is a C₁-C₂₀ alkyl, wherein any one or more —CH₂— groups of the C₁-C₂₀ alkyl is each optionally replaced independently with —C(═O)—, —CF₂—, or a heteroatomic moiety selected from the group consisting of —O—, —S—, —NH—, and —N(C₁-C₆ alkyl)-, wherein any one or more —CH₃ groups of the C₁-C₂₀ alkyl is each optionally replaced independently with —CF₃, —CF₂H, —CH₂F, or a heteroatomic moiety selected from the group consisting of —NH(C₁-C₆ alkyl), —N(C₁-C₆ alkyl)₂, —O(C₁-C₆ alkyl), and —S(C₁-C₆ alkyl), and wherein any two heteroatomic moieties are separated from one another by at least two carbon atoms;
   L is a C₂-C₅₀ alkyl, C₂-C₅₀ alkenyl, C₂-C₅₀ alkynyl, C₃-C₈ cycloalkyl, aryl, heteroaryl, C₄-C₅₀ alkyl-cycloalkyl, C₇-C₅₀ alkyl-aryl, or C₆-C₅₀ alkyl-heteroaryl, wherein any one or more —CH₂— groups of L is each optionally replaced independently with —C(═O)—, —CF₂—, or a heteroatomic moiety selected from the group consisting of —O—, —S—, —NH—, and —N(C₁-C₆ alkyl)-, wherein any one or more —CH₃ groups of L is each optionally replaced independently with —CF₃, —CF₂H, —CH₂F, or a heteroatomic moiety selected from the group consisting of —NH(C₁-C₆ alkyl), —N(C₁-C₆ alkyl)₂, —O(C₁-C₆ alkyl), and —S(C₁-C₆ alkyl), and wherein any two heteroatomic moieties are separated from one another by at least two carbon atoms;
   Z is a C₁-C₂₀ alkyl, wherein any one or more —CH₂— groups of the C₁-C₂₀ alkyl is each optionally replaced independently with —C(═O)—, —CF₂—, or a heteroatomic moiety selected from the group consisting of —O—, —S—, —NH—, and —N(C₁-C₆ alkyl)-, wherein any one or more —CH₃ groups of the C₁-C₂₀ alkyl is each optionally replaced independently with —CF₃, —CF₂H, —CH₂F, or a heteroatomic moiety selected from the group consisting of —NH(C₁-C₆ alkyl), —N(C₁-C₆ alkyl)₂, —O(C₁-C₆ alkyl), and —S(C₁-C₆ alkyl), and wherein any two heteroatomic moieties are separated from one another by at least two carbon atoms; and
   B is a second mRNA molecule having a 3′-end and a 5′-end, wherein B encodes the second polypeptide;
wherein Y is covalently attached to the 3′-end of A through a first linkage, and wherein Z is covalently attached to the 3′-end of B through a second linkage.

In an aspect, the present specification provides, and includes a method of delivering equimolar amounts of a first mRNA molecule and a second mRNA molecule to a cell, the method comprising a step of contacting the cell with an mRNA bioconjugate, or a pharmaceutically acceptable salt thereof, of formula (I)

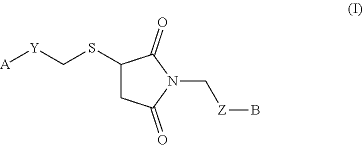

wherein
   A is a first mRNA molecule having a 3′-end and a 5′-end;
   Y is a C₁-C₂₀ alkyl, wherein any one or more —CH₂— groups of the C₁-C₂₀ alkyl is each optionally replaced independently with —C(═O)—, —CF₂—, or a heteroatomic moiety selected from the group consisting of —O—, —S—, —NH—, and —N(C₁-C₆ alkyl)-, wherein any one or more —CH₃ groups of the C₁-C₂₀ alkyl is each optionally replaced independently with —CF₃, —CF₂H, —CH₂F, or a heteroatomic moiety selected from the group consisting of —NH(C₁-C₆ alkyl), —N(C₁-C₆ alkyl)₂, —O(C₁-C₆ alkyl), and —S(C₁-C₆ alkyl), and wherein any two heteroatomic moieties are separated from one another by at least two carbon atoms;
   Z is a C₁-C₂₀ alkyl, wherein any one or more —CH₂— groups of the C₁-C₂₀ alkyl is each optionally replaced independently with —C(═O)—, —CF₂—, or a heteroatomic moiety selected from the group consisting of —O—, —S—, —NH—, and —N(C₁-C₆ alkyl)-, wherein any one or more —CH₃ groups of the C₁-C₂₀ alkyl is each optionally replaced independently with —CF₃, —CF₂H, —CH₂F, or a heteroatomic moiety selected from the group consisting of —NH(C₁-C₆ alkyl), —N(C₁-C₆ alkyl)₂, —O(C₁-C₆ alkyl), and —S(C₁-C₆ alkyl), and wherein any two heteroatomic moieties are separated from one another by at least two carbon atoms; and
   B is a second mRNA molecule having a 3′-end and a 5′-end,
wherein Y is covalently attached to the 3′-end of A through a first linkage, and wherein Z is covalently attached to the 3′-end of B through a second linkage.

In an aspect, the present specification provides, and includes a method of delivering equimolar amounts of a first mRNA molecule and a second mRNA molecule to a cell, the method comprising a step of contacting the cell with an mRNA bioconjugate, or a pharmaceutically acceptable salt thereof, of formula (II)

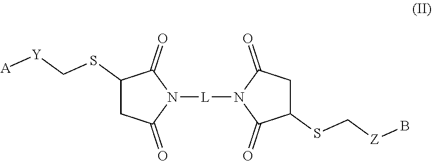

wherein
   A is a first mRNA molecule having a 3′-end and a 5′-end;
   Y is a C₁-C₂₀ alkyl, wherein any one or more —CH₂— groups of the C₁-C₂₀ alkyl is each optionally replaced independently with —C(═O)—, —CF₂—, or a heteroatomic moiety selected from the group consisting of —O—, —S—, —NH—, and —N(C₁-C₆ alkyl)-, wherein any one or more —CH₃ groups of the C₁-C₂₀ alkyl is each optionally replaced independently with —CF₃, —CF₂H, —CH₂F, or a heteroatomic moiety selected from the group consisting of —NH(C₁-C₆ alkyl), —N(C₁-C₆ alkyl)₂, —O(C₁-C₆ alkyl), and —S(C₁-C₆ alkyl), and wherein any two heteroatomic moieties are separated from one another by at least two carbon atoms;
   L is a C₂-C₅₀ alkyl, C₂-C₅₀ alkenyl, C₂-C₅₀ alkynyl, C₃-C₈ cycloalkyl, aryl, heteroaryl, C₄-C₅₀ alkyl-cycloalkyl, C₇-C₅₀ alkyl-aryl, or C₆-C₅₀ alkyl-heteroaryl, wherein any one or more —CH₂— groups of L is each optionally replaced independently with —C(═O)—, —CF₂—, or a heteroatomic moiety selected from the group consisting of —O—, —S—, —NH—, and —N(C₁-C₆ alkyl)-, wherein any one or more —CH₃ groups of L is each optionally replaced independently with —CF₃, —CF₂H, —CH₂F, or a heteroatomic moiety selected from the group consisting of —NH(C₁-C₆ alkyl), —N(C₁-C₆alkyl)₂, —O(C₁-C₆ alkyl), and —S(C₁-C₆ alkyl), and wherein any two heteroatomic moieties are separated from one another by at least two carbon atoms;
   Z is a C₁-C₂₀ alkyl, wherein any one or more —CH₂— groups of the C₁-C₂₀ alkyl is each optionally replaced independently with —C(═O)—, —CF₂—, or a heteroatomic moiety selected from the group consisting of —O—, —S—, —NH—, and —N(C₁-C₆ alkyl)-, wherein any one or more —CH₃ groups of the C₁-C₂₀ alkyl is each optionally replaced independently with —CF₃, —CF₂H, —CH₂F, or a heteroatomic moiety selected from the group consisting of —NH(C₁-C₆ alkyl), —N(C₁-C₆ alkyl)₂, —O(C₁-C₆ alkyl), and —S(C₁-C₆ alkyl), and wherein any two heteroatomic moieties are separated from one another by at least two carbon atoms; and
   B is a second mRNA molecule having a 3′-end and a 5′-end;
wherein Y is covalently attached to the 3′-end of A through a first linkage, and wherein Z is covalently attached to the 3′-end of B through a second linkage.

In an aspect, the present specification provides, and includes a method of targeted therapy, the method comprising a step of administering to a patient in need thereof a pharmaceutical formulation comprising an mRNA bioconjugate, or a pharmaceutically acceptable salt thereof, of formula (I)

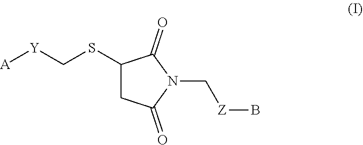

and a pharmaceutically acceptable carrier, excipient, or diluent,
wherein
   A is a first mRNA molecule having a 3′-end and a 5′-end, wherein A encodes a first therapeutic polypeptide;
   Y is a C₁-C₂₀ alkyl, wherein any one or more —CH₂— groups of the C₁-C₂₀ alkyl is each optionally replaced independently with —C(═O)—, —CF₂—, or a heteroatomic moiety selected from the group consisting of —O—, —S—, —NH—, and —N(C₁-C₆ alkyl)-, wherein any one or more —CH₃ groups of the C₁-C₂₀ alkyl is each optionally replaced independently with —CF₃, —CF₂H, —CH₂F, or a heteroatomic moiety selected from the group consisting of —NH(C₁-C₆ alkyl), —N(C₁-C₆ alkyl)₂, —O(C₁-C₆ alkyl), and —S(C₁-C₆ alkyl), and wherein any two heteroatomic moieties are separated from one another by at least two carbon atoms;
   Z is a C₁-C₂₀ alkyl, wherein any one or more —CH₂— groups of the C₁-C₂₀ alkyl is each optionally replaced independently with —C(═O)—, —CF₂—, or a heteroatomic moiety selected from the group consisting of —O—, —S—, —NH—, and —N(C₁-C₆ alkyl)-, wherein any one or more —CH₃ groups of the C₁-C₂₀ alkyl is each optionally replaced independently with —CF₃, —CF₂H, —CH₂F, or a heteroatomic moiety selected from the group consisting of —NH(C₁-C₆ alkyl), —N(C₁-C₆ alkyl)₂, —O(C₁-C₆ alkyl), and —S(C₁-C₆ alkyl), and wherein any two heteroatomic moieties are separated from one another by at least two carbon atoms; and
   B is a second therapeutic polypeptide, a therapeutic small molecule, or a second mRNA molecule having a 3′-end and a 5′-end encoding a second therapeutic polypeptide,
wherein Y is covalently attached to the 3′-end of A through a first linkage.

In an aspect, the present specification provides, and includes a method of targeted therapy, the method comprising a step of administering to a patient in need thereof a pharmaceutical formulation comprising an mRNA bioconjugate, or a pharmaceutically acceptable salt thereof, of formula (II)

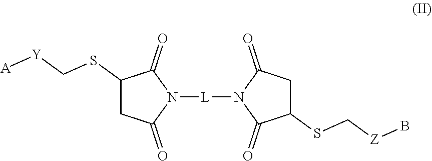

and a pharmaceutically acceptable carrier, excipient, or diluent,
wherein
   A is a first mRNA molecule having a 3′-end and a 5′-end, wherein A encodes a first therapeutic polypeptide;
   Y is a C₁-C₂₀ alkyl, wherein any one or more —CH₂— groups of the C₁-C₂₀ alkyl is each optionally replaced independently with —C(═O)—, —CF₂—, or a heteroatomic moiety selected from the group consisting of —O—, —S—, —NH—, and —N(C₁-C₆ alkyl)-, wherein any one or more —CH₃ groups of the C₁-C₂₀ alkyl is each optionally replaced independently with —CF₃, —CF₂H, —CH₂F, or a heteroatomic moiety selected from the group consisting of —NH(C₁-C₆ alkyl), —N(C₁-C₆ alkyl)₂, —O(C₁-C₆ alkyl), and —S(C₁-C₆ alkyl), and wherein any two heteroatomic moieties are separated from one another by at least two carbon atoms;
   L is a C₂-C₅₀ alkyl, C₂-C₅₀ alkenyl, C₂-C₅₀ alkynyl, C₃-C₈ cycloalkyl, aryl, heteroaryl, C₄-C₅₀ alkyl-cycloalkyl, C₇-C₅₀ alkyl-aryl, or C₆-C₅₀ alkyl-heteroaryl, wherein any one or more —CH₂— groups of L is each optionally replaced independently with —C(═O)—, —CF₂—, or a heteroatomic moiety selected from the group consisting of —O—, —S—, —NH—, and —N(C₁-C₆ alkyl)-, wherein any one or more —CH₃ groups of L is each optionally replaced independently with —CF₃, —CF₂H, —CH₂F, or a heteroatomic moiety selected from the group consisting of —NH(C₁-C₆ alkyl), —N(C₁-C₆ alkyl)₂, —O(C₁-C₆ alkyl), and —S(C₁-C₆ alkyl), and wherein any two heteroatomic moieties are separated from one another by at least two carbon atoms;
   Z is a C₁-C₂₀ alkyl, wherein any one or more —CH₂— groups of the C₁-C₂₀ alkyl is each optionally replaced independently with —C(═O)—, —CF₂—, or a heteroatomic moiety selected from the group consisting of —O—, —S—, —NH—, and —N(C₁-C₆ alkyl)-, wherein any one or more —CH₃ groups of the C₁-C₂₀ alkyl is each optionally replaced independently with —CF₃, —CF₂H, —CH₂F, or a heteroatomic moiety selected from the group consisting of —NH(C₁-C₆ alkyl), —N(C₁-C₆ alkyl)₂, —O(C₁-C₆ alkyl), and —S(C₁-C₆ alkyl), and wherein any two heteroatomic moieties are separated from one another by at least two carbon atoms; and
   B is a second therapeutic polypeptide, a therapeutic small molecule, or a second mRNA molecule having a 3′-end and a 5′-end encoding a second therapeutic polypeptide,
wherein Y is covalently attached to the 3′-end of A through a first linkage.

In an aspect, the present specification provides, and includes a method of treating, preventing, slowing the progression, or reducing the severity of cancer in a patient in need thereof, the method comprising a step of administering to the patient in need thereof a pharmaceutical formulation comprising an mRNA bioconjugate, or a pharmaceutically acceptable salt thereof, of

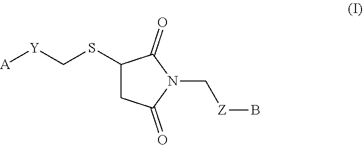

and a pharmaceutically acceptable carrier, excipient, or diluent,
wherein
   A is a first mRNA molecule having a 3′-end and a 5′-end, wherein A encodes a first therapeutic polypeptide;
   Y is a C₁-C₂₀ alkyl, wherein any one or more —CH₂— groups of the C₁-C₂₀ alkyl is each optionally replaced independently with —C(═O)—, —CF₂—, or a heteroatomic moiety selected from the group consisting of —O—, —S—, —NH—, and —N(C₁-C₆ alkyl)-, wherein any one or more —CH₃ groups of the C₁-C₂₀ alkyl is each optionally replaced independently with —CF₃, —CF₂H, —CH₂F, or a heteroatomic moiety selected from the group consisting of —NH(C₁-C₆ alkyl), —N(C₁-C₆ alkyl)₂, —O(C₁-C₆ alkyl), and —S(C₁-C₆ alkyl), and wherein any two heteroatomic moieties are separated from one another by at least two carbon atoms;
   Z is a C₁-C₂₀ alkyl, wherein any one or more —CH₂— groups of the C₁-C₂₀ alkyl is each optionally replaced independently with —C(═O)—, —CF₂—, or a heteroatomic moiety selected from the group consisting of —O—, —S—, —NH—, and —N(C₁-C₆ alkyl)-, wherein any one or more —CH₃ groups of the C₁-C₂₀ alkyl is each optionally replaced independently with —CF₃, —CF₂H, —CH₂F, or a heteroatomic moiety selected from the group consisting of —NH(C₁-C₆ alkyl), —N(C₁-C₆ alkyl)₂, —O(C₁-C₆ alkyl), and —S(C₁-C₆ alkyl), and wherein any two heteroatomic moieties are separated from one another by at least two carbon atoms; and
   B is a second therapeutic polypeptide, a therapeutic small molecule, or a second mRNA molecule having a 3′-end and a 5′-end encoding a second therapeutic polypeptide,
wherein Y is covalently attached to the 3′-end of A through a first linkage.

In an aspect, the present specification provides, and includes a method of treating, preventing, slowing the progression, or reducing the severity of cancer in a patient in need thereof, the method comprising a step of administering to the patient in need thereof a pharmaceutical formulation comprising an mRNA bioconjugate, or a pharmaceutically acceptable salt thereof, of formula (II)

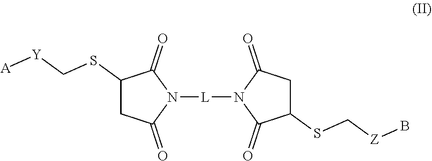

and a pharmaceutically acceptable carrier, excipient, or diluent,
wherein
   A is a first mRNA molecule having a 3′-end and a 5′-end, wherein A encodes a first therapeutic polypeptide;
   Y is a C₁-C₂₀ alkyl, wherein any one or more —CH₂— groups of the C₁-C₂₀ alkyl is each optionally replaced independently with —C(═O)—, —CF₂—, or a heteroatomic moiety selected from the group consisting of —O—, —S—, —NH—, and —N(C₁-C₆ alkyl)-, wherein any one or more —CH₃ groups of the C₁-C₂₀ alkyl is each optionally replaced independently with —CF₃, —CF₂H, —CH₂F, or a heteroatomic moiety selected from the group consisting of —NH(C₁-C₆ alkyl), —N(C₁-C₆ alkyl)₂, —O(C₁-C₆ alkyl), and —S(C₁-C₆ alkyl), and wherein any two heteroatomic moieties are separated from one another by at least two carbon atoms;
   L is a C₂-C₅₀ alkyl, C₂-C₅₀ alkenyl, C₂-C₅₀ alkynyl, C₃-C₈ cycloalkyl, aryl, heteroaryl, C₄-C₅₀ alkyl-cycloalkyl, C₇-C₅₀ alkyl-aryl, or C₆-C₅₀ alkyl-heteroaryl, wherein any one or more —CH₂— groups of the C₂-C₅₀ alkyl is each optionally replaced independently with —C(═O)—, —CF₂—, or a heteroatomic moiety selected from the group consisting of —O—, —S—, —NH—, and —N(C₁-C₆ alkyl)-, wherein any one or more —CH₃ groups of L is each optionally replaced independently with —CF₃, —CF₂H, —CH₂F, or a heteroatomic moiety selected from the group consisting of —NH(C₁-C₆ alkyl), —N(C₁-C₆ alkyl)₂, —O(C₁-C₆ alkyl), and —S(C₁-C₆ alkyl), and wherein any two heteroatomic moieties are separated from one another by at least two carbon atoms;
   Z is a C₁-C₂₀ alkyl, wherein any one or more —CH₂— groups of the C₁-C₂₀ alkyl is each optionally replaced independently with —C(═O)—, —CF₂—, or a heteroatomic moiety selected from the group consisting of —O—, —S—, —NH—, and —N(C₁-C₆ alkyl)-, wherein any one or more —CH₃ groups of the C₁-C₂₀ alkyl is each optionally replaced independently with —CF₃, —CF₂H, —CH₂F, or a heteroatomic moiety selected from the group consisting of —NH(C₁-C₆ alkyl), —N(C₁-C₆ alkyl)₂, —O(C₁-C₆ alkyl), and —S(C₁-C₆ alkyl), and wherein any two heteroatomic moieties are separated from one another by at least two carbon atoms; and
   B is a second therapeutic polypeptide, a therapeutic small molecule, or a second mRNA molecule having a 3′-end and a 5′-end encoding a second therapeutic polypeptide,
wherein Y is covalently attached to the 3′-end of A through a first linkage.

In an aspect, the present specification provides, and includes a method of treating, preventing, slowing the progression, or reducing the severity of obesity in a patient in need thereof, the method comprising a step of administering to the patient in need thereof a pharmaceutical formulation comprising an mRNA bioconjugate, or a pharmaceutically acceptable salt thereof, of formula (I)

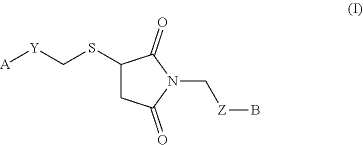

and a pharmaceutically acceptable carrier, excipient, or diluent,
wherein
   A is a first mRNA molecule having a 3′-end and a 5′-end, wherein A encodes a first therapeutic polypeptide;
   Y is a C₁-C₂₀ alkyl, wherein any one or more —CH₂— groups of the C₁-C₂₀ alkyl is each optionally replaced independently with —C(═O)—, —CF₂—, or a heteroatomic moiety selected from the group consisting of —O—, —S—, —NH—, and —N(C₁-C₆ alkyl)-, wherein any one or more —CH₃ groups of the C₁-C₂₀ alkyl is each optionally replaced independently with —CF₃, —CF₂H, —CH₂F, or a heteroatomic moiety selected from the group consisting of —NH(C₁-C₆ alkyl), —N(C₁-C₆ alkyl)₂, —O(C₁-C₆ alkyl), and —S(C₁-C₆ alkyl), and wherein any two heteroatomic moieties are separated from one another by at least two carbon atoms;
   Z is a C₁-C₂₀ alkyl, wherein any one or more —CH₂— groups of the C₁-C₂₀ alkyl is each optionally replaced independently with —C(═O)—, —CF₂—, or a heteroatomic moiety selected from the group consisting of —O—, —S—, —NH—, and —N(C₁-C₆ alkyl)-, wherein any one or more —CH₃ groups of the C₁-C₂₀ alkyl is each optionally replaced independently with —CF₃, —CF₂H, —CH₂F, or a heteroatomic moiety selected from the group consisting of —NH(C₁-C₆ alkyl), —N(C₁-C₆ alkyl)₂, —O(C₁-C₆ alkyl), and —S(C₁-C₆ alkyl), and wherein any two heteroatomic moieties are separated from one another by at least two carbon atoms; and
   B is a second therapeutic polypeptide, a therapeutic small molecule, or a second mRNA molecule having a 3′-end and a 5′-end encoding a second therapeutic polypeptide,
wherein Y is covalently attached to the 3′-end of A through a first linkage.

In an aspect, the present specification provides, and includes a method of treating, preventing, slowing the progression, or reducing the severity of obesity in a patient in need thereof, the method comprising a step of administering to the patient in need thereof a pharmaceutical formulation comprising an mRNA bioconjugate, or a pharmaceutically acceptable salt thereof, of formula (II)

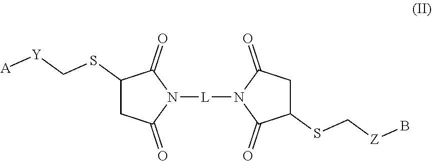

and a pharmaceutically acceptable carrier, excipient, or diluent,
wherein
   A is a first mRNA molecule having a 3′-end and a 5′-end, wherein A encodes a first therapeutic polypeptide;
   Y is a C₁-C₂₀ alkyl, wherein any one or more —CH₂— groups of the C₁-C₂₀ alkyl is each optionally replaced independently with —C(═O)—, —CF₂—, or a heteroatomic moiety selected from the group consisting of —O—, —S—, —NH—, and —N(C₁-C₆ alkyl)-, wherein any one or more —CH₃ groups of the C₁-C₂₀ alkyl is each optionally replaced independently with —CF₃, —CF₂H, —CH₂F, or a heteroatomic moiety selected from the group consisting of —NH(C₁-C₆ alkyl), —N(C₁-C₆ alkyl)₂, —O(C₁-C₆ alkyl), and —S(C₁-C₆ alkyl), and wherein any two heteroatomic moieties are separated from one another by at least two carbon atoms;
   L is a C₂-C₅₀ alkyl, C₂-C₅₀ alkenyl, C₂-C₅₀ alkynyl, C₃-C₈ cycloalkyl, aryl, heteroaryl, C₄-C₅₀ alkyl-cycloalkyl, C₇-C₅₀ alkyl-aryl, or C₆-C₅₀ alkyl-heteroaryl, wherein any one or more —CH₂— groups of the C₂-C₅₀ alkyl is each optionally replaced independently with —C(═O)—, —CF₂—, or a heteroatomic moiety selected from the group consisting of —O—, —S—, —NH—, and —N(C₁-C₆ alkyl)-, wherein any one or more —CH₃ groups of L is each optionally replaced independently with —CF₃, —CF₂H, —CH₂F, or a heteroatomic moiety selected from the group consisting of —NH(C₁-C₆ alkyl), —N(C₁-C₆ alkyl)₂, —O(C₁-C₆ alkyl), and —S(C₁-C₆ alkyl), and wherein any two heteroatomic moieties are separated from one another by at least two carbon atoms;
   Z is a C₁-C₂₀ alkyl, wherein any one or more —CH₂— groups of the C₁-C₂₀ alkyl is each optionally replaced independently with —C(═O)—, —CF₂—, or a heteroatomic moiety selected from the group consisting of —O—, —S—, —NH—, and —N(C₁-C₆ alkyl)-, wherein any one or more —CH₃ groups of the C₁-C₂₀ alkyl is each optionally replaced independently with —CF₃, —CF₂H, —CH₂F, or a heteroatomic moiety selected from the group consisting of —NH(C₁-C₆ alkyl), —N(C₁-C₆ alkyl)₂, —O(C₁-C₆ alkyl), and —S(C₁-C₆ alkyl), and wherein any two heteroatomic moieties are separated from one another by at least two carbon atoms; and
   B is a second therapeutic polypeptide, a therapeutic small molecule, or a second mRNA molecule having a 3′-end and a 5′-end encoding a second therapeutic polypeptide,
wherein Y is covalently attached to the 3′-end of A through a first linkage.

A method of enzyme replacement therapy, the method comprising a step of administering to a patient in need of enzyme replacement therapy a pharmaceutical formulation comprising an mRNA bioconjugate, or a pharmaceutically acceptable salt thereof, of formula (I)

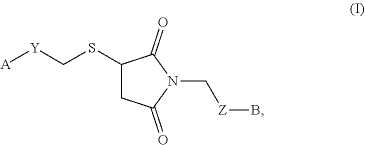

and a pharmaceutically acceptable carrier, excipient, or diluent,
wherein
   A is a first mRNA molecule having a 3′-end and a 5′-end, wherein A encodes a first enzyme;
   Y is a C₁-C₂₀ alkyl, wherein any one or more —CH₂— groups of the C₁-C₂₀ alkyl is each optionally replaced independently with —C(═O)—, —CF₂—, or a heteroatomic moiety selected from the group consisting of —O—, —S—, —NH—, and —N(C₁-C₆ alkyl)-, wherein any one or more —CH₃ groups of the C₁-C₂₀ alkyl is each optionally replaced independently with —CF₃, —CF₂H, —CH₂F, or a heteroatomic moiety selected from the group consisting of —NH(C₁-C₆ alkyl), —N(C₁-C₆ alkyl)₂, —O(C₁-C₆ alkyl), and —S(C₁-C₆ alkyl), and wherein any two heteroatomic moieties are separated from one another by at least two carbon atoms;
   Z is a C₁-C₂₀ alkyl, wherein any one or more —CH₂— groups of the C₁-C₂₀ alkyl is each optionally replaced independently with —C(═O)—, —CF₂—, or a heteroatomic moiety selected from the group consisting of —O—, —S—, —NH—, and —N(C₁-C₆ alkyl)-, wherein any one or more —CH₃ groups of the C₁-C₂₀ alkyl is each optionally replaced independently with —CF₃, —CF₂H, —CH₂F, or a heteroatomic moiety selected from the group consisting of —NH(C₁-C₆ alkyl), —N(C₁-C₆ alkyl)₂, —O(C₁-C₆ alkyl), and —S(C₁-C₆ alkyl), and wherein any two heteroatomic moieties are separated from one another by at least two carbon atoms; and
   B is an oligonucleotide, a polypeptide, a protein, a second enzyme, a small molecule, a carbohydrate, a lipid, a polyethylene glycol (PEG) molecule, or a biopolymer,
wherein Y is covalently attached to the 3′-end of A through a first linkage, and wherein the patient in need of enzyme replacement therapy is deficient of the first enzyme.

A method of enzyme replacement therapy, the method comprising a step of administering to a patient in need of enzyme replacement therapy a pharmaceutical formulation comprising an mRNA bioconjugate, or a pharmaceutically acceptable salt thereof, of formula (II)

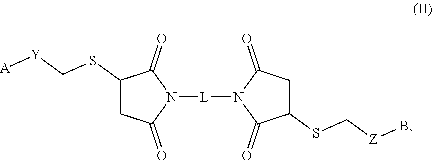

and a pharmaceutically acceptable carrier, excipient, or diluent,
wherein
   A is a first mRNA molecule having a 3′-end and a 5′-end, wherein A encodes a first enzyme;
   Y is a C₁-C₂₀ alkyl, wherein any one or more —CH₂— groups of the C₁-C₂₀ alkyl is each optionally replaced independently with —C(═O)—, —CF₂—, or a heteroatomic moiety selected from the group consisting of —O—, —S—, —NH—, and —N(C₁-C₆ alkyl)-, wherein any one or more —CH₃ groups of the C₁-C₂₀ alkyl is each optionally replaced independently with —CF₃, —CF₂H, —CH₂F, or a heteroatomic moiety selected from the group consisting of —NH(C₁-C₆ alkyl), —N(C₁-C₆ alkyl)₂, —O(C₁-C₆ alkyl), and —S(C₁-C₆ alkyl), and wherein any two heteroatomic moieties are separated from one another by at least two carbon atoms;
   L is a C₂-C₅₀ alkyl, C₂-C₅₀ alkenyl, C₂-C₅₀ alkynyl, C₃-C₈ cycloalkyl, aryl, heteroaryl, C₄-C₅₀ alkyl-cycloalkyl, C₇-C₅₀ alkyl-aryl, or C₆-C₅₀ alkyl-heteroaryl, wherein any one or more —CH₂— groups of the C₂-C₅₀ alkyl is each optionally replaced independently with —C(═O)—, —CF₂—, or a heteroatomic moiety selected from the group consisting of —O—, —S—, —NH—, and —N(C₁-C₆ alkyl)-, wherein any one or more —CH₃ groups of L is each optionally replaced independently with —CF₃, —CF₂H, —CH₂F, or a heteroatomic moiety selected from the group consisting of —NH(C₁-C₆ alkyl), —N(C₁-C₆ alkyl)₂, —O(C₁-C₆ alkyl), and —S(C₁-C₆ alkyl), and wherein any two heteroatomic moieties are separated from one another by at least two carbon atoms;
   Z is a C₁-C₂₀ alkyl, wherein any one or more —CH₂— groups of the C₁-C₂₀ alkyl is each optionally replaced independently with —C(═O)—, —CF₂—, or a heteroatomic moiety selected from the group consisting of —O—, —S—, —NH—, and —N(C₁-C₆ alkyl)-, wherein any one or more —CH₃ groups of the C₁-C₂₀ alkyl is each optionally replaced independently with —CF₃, —CF₂H, —CH₂F, or a heteroatomic moiety selected from the group consisting of —NH(C₁-C₆ alkyl), —N(C₁-C₆ alkyl)₂, —O(C₁-C₆ alkyl), and —S(C₁-C₆ alkyl), and wherein any two heteroatomic moieties are separated from one another by at least two carbon atoms; and
   B is an oligonucleotide, a polypeptide, a protein, a second enzyme, a small molecule, a carbohydrate, a lipid, a polyethylene glycol (PEG) molecule, or a biopolymer,
wherein Y is covalently attached to the 3′-end of A through a first linkage, and wherein the patient in need of enzyme replacement therapy is deficient of the first enzyme.

In an aspect, the present specification provides, and includes use of a pharmaceutical formulation comprising an mRNA bioconjugate, or a pharmaceutically acceptable salt thereof, of any one of formulae (I), (II), (III), (IV), or (V) and a pharmaceutically acceptable carrier, excipient, or diluent for targeted therapy in a patient in need thereof. In an aspect, the present specification provides, and includes use of a pharmaceutical formulation comprising an mRNA bioconjugate, or a pharmaceutically acceptable salt thereof, of any one of formulae (I), (II), (III), (IV), or (V) and a pharmaceutically acceptable carrier, excipient, or diluent for treating, preventing, slowing the progression, or reducing the severity of cancer in a patient in need thereof. In an aspect, the present specification provides, and includes use of a pharmaceutical formulation comprising an mRNA bioconjugate, or a pharmaceutically acceptable salt thereof, of any one of formulae (I), (II), (III), (IV), or (V) and a pharmaceutically acceptable carrier, excipient, or diluent for treating, preventing, slowing the progression, or reducing the severity of obesity or one or more other metabolic and/or cardiovascular disorders in a patient in need thereof. In an aspect, the present specification provides, and includes use of a pharmaceutical formulation comprising an mRNA bioconjugate, or a pharmaceutically acceptable salt thereof, of any one of formulae (I), (II), (III), (IV), or (V) and a pharmaceutically acceptable carrier, excipient, or diluent for enzyme replacement therapy in a patient in need thereof.

In an aspect, the present specification provides, and includes use of an mRNA bioconjugate, or a pharmaceutically acceptable salt thereof, of any one of formulae (I), (II), (III), (IV), or (V) for the manufacture of a medicament for targeted therapy in a patient in need thereof. In an aspect, the present specification provides, and includes use of an mRNA bioconjugate, or a pharmaceutically acceptable salt thereof, of any one of formulae (I), (II), (III), (IV), or (V) for the manufacture of a medicament for treating, preventing, slowing the progression, or reducing the severity of cancer in a patient in need thereof. In an aspect, the present specification provides, and includes use of an mRNA bioconjugate, or a pharmaceutically acceptable salt thereof, of any one of formulae (I), (II), (III), (IV), or (V) for the manufacture of a medicament for treating, preventing, slowing the progression, or reducing the severity of obesity or one or more other metabolic and/or cardiovascular disorders in a patient in need thereof. In an aspect, the present specification provides, and includes use of an mRNA bioconjugate, or a pharmaceutically acceptable salt thereof, of any one of formulae (I), (II), (III), (IV), or (V) for the manufacture of a medicament for enzyme replacement therapy in a patient in need thereof.

### BRIEF DESCRIPTION OF THE DRAWINGS

FIG. 1A shows the structure of PolyA-γ-mercaptopropanol.

FIG. 1B shows a HPLC chromatogram of PolyA-γ-mercaptopropanol obtained from a vendor.

FIG. 1C shows a mass spectrum of PolyA-γ-mercaptopropanol obtained from a vendor.

FIG. 2A shows the reaction converting PolyA-γ-mercaptopropanol to PolyA-SH.

FIG. 2B shows the UV absorbance chromatogram at 260 nm of the reaction converting PolyA-γ-mercaptopropanol to PolyA-SH. The PolyA-SH peaks are highlighted.

FIG. 2C shows the UV absorbance chromatogram at 260 nm of the reaction converting PolyA-γ-mercaptopropanol to PolyA-SH. The PolyA-γ-mercaptopropanol peaks are highlighted.

FIG. 2D shows the total ion count mass spectrum of PolyA-SH.

FIG. 2E shows the total ion count of purified PolyA-γ-mercaptopropanol.

FIG. 2F shows the structure of non-canonical PolyA-γ-mercaptopropanol.

FIG. 2G shows the UV absorbance chromatogram at 260 nm of non-canonical PolyA-γ-mercaptopropanol.

FIG. 2H shows the mass spectrogram of non-canonical PolyA-γ-mercaptopropanol.

FIG. 2I shows the structure of non-canonical PolyA-dideoxynucleotide.

FIG. 2J shows the UV absorbance chromatogram at 260 nm of non-canonical PolyA-dideoxynucleotide.

FIG. 2K shows the mass spectrogram of non-canonical PolyA-dideoxynucleotide.

FIG. 3A shows the reaction converting PolyA-SH to PolyA-S-Maleimide-SulfoCy5.

FIG. 3B shows the total ion count chromatogram of PolyA-S-Maleimide-SulfoCy5.

FIG. 3C shows the mass spectrum of PolyA-S-Maleimide-SulfoCy5.

FIG. 3D shows the mass spectrum of the peak highlighted in FIG. 3B.

FIG. 4A shows the reaction converting PolyA-SH to PolyA-S-Maleimide-PEG19-Maleimide.

FIG. 4B shows the UV absorbance chromatogram of PolyA-S-Maleimide-PEG19-Maleimide.

FIG. 4C shows the total ion count chromatogram of PolyA-S-Maleimide-PEG19-Maleimide.

FIG. 4D shows the mass spectrum of PolyA-S-Maleimide-PEG19-Maleimide.

FIG. 4E shows the extracted mass spectrum peak representing PolyA-S-Maleimide-PEG19-Maleimide (unhydrolyzed)

FIG. 5A shows the reaction converting PolyA-SH to PolyA-S-Maleimide-PEG19-Maleimide-S-PolyA.

FIG. 5B shows the UV absorbance chromatogram of PolyA-S-Maleimide-PEG19-Maleimide-S-PolyA.

FIG. 5C shows the total ion count chromatogram of PolyA-S-Maleimide-PEG19-Maleimide-S-PolyA.

FIG. 5D shows the mass spectrum of PolyA-S-Maleimide-PEG19-Maleimide-S-PolyA.

FIG. 6A shows the reaction converting eGFP mRNA to eGFP-SH through enzymatic ligation by T4 RNA Ligase.

FIG. 6B shows the UV absorbance chromatogram of eGFP (top) and eGFP-SH (bottom).

FIG. 6C shows the RP-HPLC chromatogram at an absorbance wavelength of 260 nm of eGFP-SH after 12 h at 4° C. (top); and eGFP-SH after reduction conditions and isolation using Purification Method 3 (bottom).

FIG. 7A shows the reaction converting mCherry mRNA to mCherry-SH through enzymatic ligation by T4 RNA Ligase.

FIG. 7B shows the UV absorbance chromatogram of mCherry (top) and mCherry-SH (bottom).

FIG. 7C shows the UV absorbance chromatogram at an absorbance wavelength of 260 nm of mCherry-SH after 12 h at 4° C. (top); and mCherry-SH after reduction conditions and isolation using Purification Method 3 (bottom).

FIG. 8A shows the reaction converting FLuc (Firefly Luciferase) mRNA to FLuc-SH through enzymatic ligation by T4 RNA Ligase.

FIG. 8B shows the UV absorbance chromatogram of Firefly FLuc (top) and FLuc-SH (bottom).

FIG. 8C shows FLuc mock ligation.

FIG. 8D shows the UV absorbance chromatogram of FLuc mock ligation.

FIG. 8E shows the reaction converting FLuc to FLuc-NC-ddnt.

FIG. 8F shows the UV absorbance chromatogram of FLuc (top) and FLuc-NC-ddnt (bottom).

FIG. 8G shows the reaction converting FLuc to FLuc-NC-SR.

FIG. 8H shows the reaction converting FLuc (top) to FLuc-NC-SR (bottom).

FIG. 8I shows the reaction converting FLuc to FLuc-NC-SH.

FIG. 8J shows the reaction converting FLuc (top), FLuc-NC-SR after the ligation step but before the DTT reduction step, and FLuc-NC-SH (bottom).

FIG. 9A shows the reaction converting eGFP-SH to eGFP-S-Maleimide-SulfoCy5 by chemical modification.

FIG. 9B shows the UV absorbance chromatogram of eGFP-SH (top) and eGFP-S-Maleimide-SulfoCy5 (bottom).

FIG. 10A shows the reaction converting eGFP to eGFP-S-Maleimide-SulfoCy5 by late-stage enzymatic modification.

FIG. 10B shows a UV absorbance chromatogram of background (top) and eGFP-S-Maleimide-SulfoCy5 (bottom) produced from enzymatic ligation of eGFP with PolyA-S-Maleimide-SulfoCy5 at 16.57 min retention time.

FIG. 11A shows the reaction converting mCherry-SH to mCherry-S-Maleimide-SulfoCy5 by chemical modification.

FIG. 11B shows the UV absorbance chromatogram of mCherry-SH (top) and mCherry-S-Maleimide-SulfoCy5 (bottom).

FIG. 12A shows the reaction converting FLuc-SH to FLuc-S-Maleimide-SulfoCy5 by chemical modification.

FIG. 12B shows the UV absorbance chromatogram of FLuc-SH (top) and FLuc-S-Maleimide-SulfoCy5 (bottom).

FIG. 13A shows the reaction converting FLuc to FLuc-S-Maleimide-SulfoCy5 by late-stage enzymatic modification.

FIG. 13B shows the UV absorbance chromatogram of the single step reaction shown in FIG. 13A to form FLuc-S-Maleimide-SulfoCy5 (bottom) from FLuc (top).

FIG. 13C shows structure of Tris-GalNAc-β-Ala-PEG3-Maleimide.

FIG. 13D shows abbreviated structure of Tris-GalNAc-3-Ala-PEG3-Maleimide.

FIG. 13E shows the UV absorbance chromatogram for FLuc-SH (top) and FLuc-S-Maleimide-GalNAc (bottom).

FIG. 13F shows the reaction converting FLuc-NC-SH to FLuc-NC-S-Maleimide-GalNAc.

FIG. 13G shows the UV absorbance chromatogram for a persistent impurity FLuc (top), FLuc-NC-SH (middle), and FLuc-S-Maleimide-GalNAc (bottom).

FIG. 14A shows the reaction converting eGFP-SH to eGFP-S-Maleimide-PEG19-Maleimide.

FIG. 14B shows the UV absorbance chromatogram of eGFP-S-Maleimide-PEG19-Maleimide as generated by the reaction shown in FIG. 14A. Peaks to the left of the main peak at 9.623 min and 10.043 min correspond to the starting material eGFP-SH.

FIG. 14C shows alternative UV absorbance chromatogram of eGFP-S-Maleimide-PEG19-Maleimide using different HPLC parameters.

FIG. 15A shows the reaction converting mCherry-SH to mCherry-S-Maleimide-PEG19-Maleimide.

FIG. 15B shows the UV absorbance chromatogram of mCherry-S-Maleimide-PEG19-Maleimide. Peaks to the left of the main peak correspond to the starting material mCherry-SH.

FIG. 16A shows the reaction of mCherry-S-Maleimide-PEG19-Maleimide with a small molecule thiol or a thiol linked to an oligonucleotide (R—SH).

FIG. 16B shows the UV absorbance chromatogram of mCherry-S-Maleimide-PEG19-Maleimide (top), the reaction of mCherry-S-Maleimide-PEG19-Maleimide with 0-mercaptoethanol (second from top), the reaction of mCherry-S-Maleimide-PEG19-Maleimide with cysteine (second from bottom), and PolyA-SH (bottom).

FIG. 17A shows the reaction converting FLuc-SH to FLuc-S-Maleimide-PEG19-Maleimide.

FIG. 17B shows the UV absorbance chromatogram of FLuc-S-Maleimide-PEG19-Maleimide. The two predominant peaks at 16.413 min and 17.087 min are likely the result of the HPLC method, as shown by the temperature-dependent study described in the examples.

FIG. 18A shows the reaction converting eGFP to eGFP-S-Maleimide-PEG19-Maleimide-S-PolyA by enzymatic ligation.

FIG. 18B shows the UV absorbance chromatogram of eGFP-S-Maleimide-PEG19-Maleimide-S-PolyA.

FIG. 19A shows the reaction converting eGFP to eGFP-S-Maleimide-PEG19-Maleimide-S-eGFP by enzymatic ligation.

FIG. 19B shows the UV absorbance chromatogram of eGFP-S-Maleimide-PEG19-Maleimide-S-eGFP. eGFP (top) is reacted with polyA-dimer to give a major peak at 10.131 min that is attributed to eGFP-S-Maleimide-PEG19-Maleimide-S-eGFP (bottom).

FIG. 20A shows the reaction converting eGFP-S-Maleimide-PEG19-Maleimide to eGFP-S-Maleimide-PEG19-Maleimide-S-mCherry.

FIG. 20B shows the UV absorbance chromatogram of eGFP-S-Maleimide-PEG19-Maleimide-S-mCherry. The crude reaction mixture (top) has peaks at 8.584 and 8.851 min that correspond to mCherry-SH, and peaks at 9.824 and 10.264 min correspond to eGFP-S-Maleimide-PEG19-Maleimide. The purified material (bottom) shows less than 5% residual mCherry-SH.

FIG. 21A shows the reaction converting eGFP-S-Maleimide-PEG19-Maleimide or mCherry-S-Maleimide-PEG19-Maleimide to eGFP-S-Maleimide-PEG19-Maleimide-S-mCherry.

FIG. 21B shows the UV absorbance chromatogram of eGFP-S-Maleimide-PEG19-Maleimide-S-mCherry. The crude reaction mixture shows peaks at 8.800 and 9.060 min that correspond to eGFP-SH, and peaks at 10.006 and 10.373 min correspond to mCherry-S-Maleimide-PEG19-Maleimide.

FIG. 21C shows the SEC-HPLC chromatograms of the eGFP-S-Maleimide-PEG19-Maleimide-S-mCherry reaction mixture.

FIG. 21D shows the capillary electrophoresis of Fractions 4 and 6.

FIG. 21E shows RP-HPLC chromatograms of eGFP, mCherry, Fraction 4, and Fraction 6.

FIG. 21F shows the RP-HPLC chromatogram at an absorbance wavelength of 260 nm of the crude reaction mixture containing eGFP-S-Maleimide-PEG19-Maleimide-S-mCherry without (top) and with (bottom) DTT treatment.

FIG. 21G shows the RP-HPLC chromatogram at an absorbance wavelength of 260 nm of another SEC-purified sample of eGFP-S-Maleimide-PEG19-Maleimide-S-mCherry.

FIG. 21H shows the area under the curve of each peak shown in FIG. 21G to determine the purity of the sample.

FIG. 22A shows the reaction converting eGFP-S-Maleimide-PEG19-Maleimide-S-PolyA to eGFP-S-Maleimide-PEG19-Maleimide-S-mCherry by late-stage enzymatic ligation.

FIG. 22B shows the UV absorbance chromatogram of eGFP-S-Maleimide-PEG19-Maleimide-S-mCherry. The crude reaction mixture (top) shows peaks corresponding to the reactant eGFP-S-Maleimide-PEG19-Maleimide-S-PolyA. The reaction mixture (bottom) shows peaks at 8.872 min and 9.119 min that correspond to mCherry.

FIG. 23A shows possible hydrolyzed and unhydrolyzed forms of PolyA-S-Maleimide-PEG19-Maleimide when the product is run at varying column temperatures.

FIG. 23B shows the amount of hydrolyzed product vs. temperature of the liquid chromatography column.

FIG. 23C shows the UV absorbance chromatogram of PolyA-S-Maleimide-PEG19-Maleimide at increasing temperatures of 20° C. (top), 37° C., 55° C., 65° C., 75° C., to 85° C. (bottom).

FIG. 23D shows the RP-HPLC chromatogram of reaction products synthesized by chemical modification and enzyme ligation.

FIG. 23E shows the size exclusion chromatogram of reaction products synthesized by chemical modification and enzyme ligation.

FIG. 24 shows the reaction of enzymatic ligation of mRNA with a modified single nucleotide followed by conjugation.

FIG. 25A shows an exemplary reaction conjugating peptides and mRNA.

FIG. 25B shows the RP-HPLC chromatogram of reaction products synthesized by enzymatic ligation and conjugation.

FIG. 25C shows the RP-HPLC chromatogram of reaction products synthesized when eGFP-Cyt-S-Maleimide-PEG19-Maleimide is conjugated with Peptide 1.

FIG. 25D shows the RP-HPLC chromatogram of reaction products synthesized when eGFP-Cyt-S-Maleimide-PEG19-Maleimide is conjugated with Peptide 2.

FIG. 25E shows the RP-HPLC chromatogram of reaction products synthesized when eGFP-Cyt-S-Maleimide-PEG19-Maleimide is conjugated with Peptide 3.

FIG. 25F shows the RP-HPLC chromatogram of reaction products synthesized when eGFP-Cyt-S-Maleimide-PEG19-Maleimide is conjugated with Peptide 4.

FIG. 25G shows the RP-HPLC chromatogram of reaction products synthesized when eGFP-Cyt-S-Maleimide-PEG19-Maleimide is conjugated with Peptide 5.

FIG. 25H shows the RP-HPLC chromatogram of reaction products synthesized when eGFP-Cyt-S-Maleimide-PEG19-Maleimide is conjugated with Peptide 6.

FIG. 25I shows the RP-HPLC chromatogram of reaction products synthesized when eGFP-Cyt-S-Maleimide-PEG19-Maleimide is conjugated with Peptide 7.

FIG. 25J shows the capillary electrophoresis of reaction products when eGFP-Cyt-S-Maleimide-PEG19-Maleimide is conjugated with Peptides 1-7.

FIG. 26 shows the RP-HPLC chromatogram when eGFP-Cyt-S-Maleimide-PEG19-Maleimide and Peptide 1 are conjugated and isolated.

FIG. 27A shows representative flow cytometry plots before gating for cells that are not fluorescent in either channel.

FIG. 27B shows representative flow cytometry plots after gating for cells that are not fluorescent in either channel.

FIG. 28A shows that majority of cells do not fluoresce (lower left quadrant).

FIG. 28B shows that majority of cells that do fluoresce (75% average) produce fluorescence in the green channel only (lower right quadrant).

FIG. 28C shows the average percentage of all gated events within a given quadrant for cells treated with eGFP mRNA.

FIG. 29A shows that majority of cells do not fluoresce (lower left quadrant).

FIG. 29B shows that about 18% of the fluorescent cells fluoresce in both red and green channels.

FIG. 29C shows the average percentage of all gated events within a given quadrant for cells treated with eGFP-S-Maleimide-SulfoCy5.

FIG. 30A shows the mean green fluorescence intensity of cells treated with eGFP mRNA and eGFP-S-Maleimide-SulfoCy5.

FIG. 30B shows the mean red fluorescence intensity of cells treated with eGFP mRNA and eGFP-S-Maleimide-SulfoCy5.

FIG. 31A shows representative flow cytometry plots before gating for cells that are not fluorescent in either channel.

FIG. 31B shows representative flow cytometry plots after gating for cells that are not fluorescent in either channel.

FIG. 31C shows the average percentage of all gated events within a given quadrant for cells treated with eGFP.

FIG. 32A shows representative flow cytometry plots before gating for cells that are not fluorescent in either channel.

FIG. 32B shows representative flow cytometry plots after gating for cells that are not fluorescent in either channel.

FIG. 32C shows the average percentage of all gated events within a given quadrant for cells treated with mCherry.

FIG. 33A shows representative flow cytometry plots before gating for cells that are not fluorescent in either channel.

FIG. 33B shows representative flow cytometry plots after gating for cells that are not fluorescent in either channel.

FIG. 33C shows the average percentage of all gated events within a given quadrant for cells treated with eGFP-S-Maleimide-PEG19-Maleimide-S-mCherry.

FIG. 34A shows the mean green fluorescence intensity of cells treated with eGFP mRNA, mCherry mRNA, and eGFP-S-Maleimide-PEG19-Maleimide-S-mCherry.

FIG. 34B shows the mean red fluorescence intensity of cells treated with eGFP mRNA, mCherry mRNA, and eGFP-S-Maleimide-PEG19-Maleimide-S-mCherry.

FIG. 35A shows representative flow cytometry plots before gating for cells that are not fluorescent in either channel.

FIG. 35B shows representative flow cytometry plots after gating for cells that are not fluorescent in either channel.

FIG. 35C shows the average percentage of all gated events within a given quadrant for cells treated with eGFP mRNA.

FIG. 36A shows representative flow cytometry plots before gating for cells that are not fluorescent in either channel.

FIG. 36B shows representative flow cytometry plots after gating for cells that are not fluorescent in either channel.

FIG. 36C shows the average percentage of all gated events within a given quadrant for cells treated with mCherry mRNA.

FIG. 37A shows representative flow cytometry plots before gating for cells that are not fluorescent in either channel.

FIG. 37B shows representative flow cytometry plots after gating for cells that are not fluorescent in either channel.

FIG. 37C shows the average percentage of all gated events within a given quadrant for cells treated with both eGFP mRNA and mCherry mRNA.

FIG. 38A shows representative flow cytometry plots before gating for cells that are not fluorescent in either channel.

FIG. 38B shows representative flow cytometry plots after gating for cells that are not fluorescent in either channel.

FIG. 38C shows the average percentage of all gated events within a given quadrant for cells treated with eGFP-S-Maleimide-PEG19-Maleimide-S-mCherry.

FIG. 39A shows the mean green fluorescence intensity of cells treated with eGFP-S-Maleimide-PEG19-Maleimide-S-mCherry and eGFP mRNA and mCherry mRNA.

FIG. 39B shows the mean red fluorescence intensity of cells treated with eGFP-S-Maleimide-PEG19-Maleimide-S-mCherry and eGFP mRNA and mCherry mRNA.

FIG. 40A shows the amount of fluorescence detected 24 hours after each sample in IVIS Study 1 was injected into the tail of a BALB/c mouse.

FIG. 40B shows the amount of fluorescence detected 24 hours after each sample in IVIS Study 1 was injected into the tail of a BALB/c mouse.

FIG. 41A shows the amount of fluorescence detected 24 hours after each sample in IVIS Study 2 was injected into the tail of a BALB/c mouse.

FIG. 41B shows the amount of fluorescence detected 24 hours after each sample in IVIS Study 2 was injected into the tail of a BALB/c mouse.

FIG. 42A shows the amount of fluorescence detected 24 hours after each sample in IVIS Study 3 was injected into the tail of a BALB/c mouse.

FIG. 42B shows the amount of fluorescence detected 24 hours after each sample in IVIS Study 3 was injected into the tail of a BALB/c mouse.

FIG. 42C shows the amount of fluorescence detected 48 hours after each sample in IVIS Study 3 was injected into the tail of a BALB/c mouse.

FIG. 42D shows the amount of fluorescence detected 48 hours after each sample in IVIS Study 3 was injected into the tail of a BALB/c mouse.

FIG. 42E shows the amount of fluorescence detected 96 hours after each sample in IVIS Study 3 was injected into the tail of a BALB/c mouse.

FIG. 42F shows the amount of fluorescence detected 96 hours after each sample in IVIS Study 3 was injected into the tail of a BALB/c mouse.

FIG. 43A shows the reaction of PembrolizumabHC with PolyA-γ-mercaptopropanol.

FIG. 43B shows the RP-HPLC chromatogram at an absorbance wavelength of 260 nm of PembrolizumabHC (top) and PembrolizumabHC-SH (bottom).

FIG. 44A shows the reaction of PembrolizumabHC with PolyA-γ-mercaptopropanol.

FIG. 44B shows the RP-HPLC chromatogram at an absorbance wavelength of 260 nm of PembrolizumabLC (top) and PembrolizumabLC-SH (bottom).

FIG. 45A shows the reaction of PembrolizumabHC-SH with bis-Maleimide-PEG19.

FIG. 45B shows the RP-HPLC chromatogram at an absorbance wavelength of 260 nm of Pembrolizumab-SH (top) and Pembrolizumab-S-Maleimide-PEG19-Maleimide (bottom).

FIG. 46A shows the reaction of PembrolizumabHC-S-Maleimide-PEG19-Maleimide with PembrolizumabHC-SH.

FIG. 46B shows the gel bands from capillary electrophoresis of components isolated from the reaction mixture of PembrolizumabHC-S-Maleimide-PEG19-Maleimide and PembrolizumabHC-SH (top).

### DETAILED DESCRIPTION

### A. Definitions

This description is not intended to be a detailed catalog of all the different ways in which the disclosure may be implemented, or all the features that may be added to the instant disclosure. For example, features illustrated with respect to one embodiment may be incorporated into other embodiment, and features illustrated with respect to a particular embodiment may be deleted from that embodiment. Thus, the disclosure contemplates that in some embodiments of the disclosure, any feature or combination of features set forth herein can be excluded or omitted. In addition, numerous variations and additions to the various embodiments suggested herein will be apparent to those skilled in the art in light of the instant disclosure, which do not depart from the instant disclosure. In other instances, well-known structures, interfaces, and processes have not been shown in detail in order not to unnecessarily obscure the invention. It is intended that no part of this specification be construed to effect a disavowal of any part of the full scope of the invention. Hence, the following descriptions are intended to illustrate some particular embodiments of the disclosure, and not to exhaustively specify all permutations, combinations and variations thereof.

Unless otherwise defined, all technical and scientific terms used herein have the same meaning as commonly understood by one of ordinary skill in the art to which this disclosure belongs. The terminology used in the description of the disclosure herein is for the purpose of describing particular aspects or embodiments only and is not intended to be limiting of the disclosure.

All publications, patent applications, patents and other references cited herein are incorporated by reference in their entireties for the teachings relevant to the sentence and/or paragraph in which the reference is presented. References to techniques employed herein are intended to refer to the techniques as commonly understood in the art, including variations on those techniques or substitutions of equivalent techniques that would be apparent to one of skill in the art.

Unless the context indicates otherwise, it is specifically intended that the various features of the disclosure described herein can be used in any combination. Moreover, the present disclosure also contemplates that in some embodiments of the disclosure, any feature or combination of features set forth herein can be excluded or omitted.

The term “and/or” when used in a list of two or more items, means that any one of the listed items can be employed by itself or in combination with any one or more of the listed items. For example, the expression “A and/or B” is intended to mean either or both of A and B, i.e., A alone, B alone, or A and B in combination. The expression “A, B and/or C” is intended to mean A alone, B alone, C alone, A and B in combination, A and C in combination, B and C in combination, or A, B, and C in combination.

As used herein, terms in the singular and the singular forms “a,” “an,” and “the,” for example, include plural referents unless the content clearly dictates otherwise.

Where a range of values is provided, it is understood that each intervening value, between the upper and lower limit of that range and any other stated or intervening value in that stated range is encompassed within the disclosure. The upper and lower limits of these smaller ranges may independently be included in the smaller ranges, and are also encompassed within the disclosure, subject to any specifically excluded limit in the stated range. Where the stated range includes one or both of the limits, ranges excluding either or both of those included limits are also included in the disclosure. Whenever the phrase “comprising” is used, variations such as “consisting essentially of” and “consisting of” are also contemplated.

Unless defined otherwise herein, terms are to be understood according to conventional usage by those of ordinary skill in the relevant art. Where a term is provided in the singular, the inventors also contemplate aspects of the disclosure described by the plural of that term. Where there are discrepancies in terms and definitions used in references that are incorporated by reference, the terms used in this application shall have the definitions given herein. Other technical terms used have their ordinary meaning in the art in which they are used, unless they are given a different definition herein.

As used herein, the term “exemplary” is used to mean serving as an example, instance, or illustration. Any embodiment or aspect described as “exemplary” is not necessarily to be construed as preferred or advantageous over other embodiments or aspects, nor is it meant to preclude equivalent structures and techniques known to those of ordinary skill in the art. Rather, use of the word exemplary is intended to present concepts in a concrete fashion, and the disclosed subject matter is not limited by such examples.

As used herein, “bioconjugate” refers to a conjugated molecule made of up of at least two constituent molecules linked by a covalent bond, where at least one of the constituent molecules is a biomolecule. In an aspect, at least one of the constituent molecules of a bioconjugate is a nucleotide. In an aspect, at least one of the constituent molecules of a bioconjugate is an oligonucleotide. In an aspect, at least one of the constituent molecules of a bioconjugate is a peptide. In an aspect, at least one of the constituent molecules of a bioconjugate is a polypeptide. In an aspect, at least one of the constituent molecules of a bioconjugate is a protein. In an aspect, at least one of the constituent molecules of a bioconjugate is a lipid. In an aspect, at least one of the constituent molecules of a bioconjugate is a carbohydrate.

As used herein, “nucleobase,” “nitrogenous base,” or “base” refers to a nitrogen-containing heterocyclic compound. In an aspect, a nucleobase comprises a simple ring. In an aspect, a nucleobase comprises a polycyclic ring. In an aspect, a nucleobase comprises a fused ring. In an aspect, a nucleobase is a purine. In an aspect, a purine base is guanine. In an aspect, a purine base is an adenine. In an aspect, a nucleobase is a pyrimidine. In an aspect, a pyrimidine is cytosine. In an aspect, a pyrimidine is a thymine. In an aspect, a nucleobase is a uracil. Nucleobases can be naturally-occurring, or non-natural. Nucleobases can also be modified. Examples of modified nucleobases include, without being limited to, hypoxanthine, xanthine, 7-methylguanine, 5,6-dihydrouracil, 5-methylcytosine, and 5-hydroxymethylcytosine.

As used herein, “nucleoside” refers to a compound composed of a nucleobase as described herein, and a five-carbon sugar. For example, the five-carbon sugar can be a ribose or a 2′-deoxyribose. Examples of nucleosides include but are not limited to adenosine, deoxyadenosine, guanosine, deoxyguanosine, 5-methyluridine, thymidine, uridine, deoxyuridine, cytidine, and deoxycytidine.

As used herein, a “nucleotide” refers to a compound composed of a nucleobase as described herein, a five-carbon sugar, and one or more phosphate groups. For example, the five-carbon sugar can be a ribose or a 2′-deoxyribose. Examples of nucleotides include but are not limited to adenine, guanine, 5-methyluridine, uridine, thymidine, and cytidine. Nucleotides can be naturally-occurring or synthetic.

As used herein, “oligonucleotides” are polymers of nucleotides. Oligonucleotides may comprise 2′-deoxyribonucleotides, ribonucleotides, and a combination thereof. In an aspect, an oligonucleotide is an RNA molecule. In an aspect, an oligonucleotide is an mRNA molecule. In an aspect, an oligonucleotide is a DNA molecule. In an aspect, an oligonucleotide is an antisense oligonucleotide.

As used herein, “oligonucleotide bioconjugate” refers to a conjugated molecule made of up of at least two constituent molecules linked by a covalent bond, where at least one of the constituent molecules is an oligonucleotide.

As used herein, “DNA” or “deoxyribonucleic acid” refers to a polymer of deoxyribonucleotides. DNA may be single-stranded, or double-stranded when two chains coil around each other to form a double helix. The nucleic acid sequence of a DNA can be naturally-occurring, e.g., genomic DNA, or synthetic.

As used herein, “RNA” or “ribonucleic acid” refers to a polymer of ribonucleotides. Without being bound by theory, RNA may be single-stranded, double-stranded, unstructured, or structured. RNA may be coding or non-coding. In an aspect, an RNA is a messenger RNA (mRNA). In an aspect, an RNA is a transfer RNA (tRNA). In an aspect, an RNA is a non-coding RNA (ncRNA). In an aspect, an RNA is a ribosomal RNA (rRNA). In an aspect, an RNA is a small nuclear RNA (snRNA). In an aspect, an RNA is a small nucleolar RNA (snoRNA). In an aspect, an RNA is a long non-coding RNA (lncRNA). In an aspect, an RNA is a microRNA (miRNA).

As used herein, “mRNA” or “messenger RNA” refers to a single-stranded RNA molecule that corresponds to the genetic sequence of a gene. Without being bound by theory, an mRNA can be read by a ribosome in the process of synthesizing a peptide or protein. As used herein, mRNA refers to both a pre-processed mRNA (pre-mRNA) where RNA splicing has not yet occurred, and processed mRNA after the introns have been removed. In an aspect, an mRNA molecule comprises a 5′-cap, a coding region, a 3′-UTR (untranslated region), and a poly-A tail. In an aspect, an mRNA molecule comprises a 5′-cap, a coding region, and a poly-A tail. In an aspect, an mRNA molecule comprises a poly-A tail.

As used herein, “mRNA bioconjugate” refers to a conjugated molecule made of up of at least two constituent molecules linked by a covalent bond, where at least one of the constituent molecules is an mRNA molecule.

As used herein, “peptide” and “polypeptide” can be used interchangeably, and refer to a chain of amino acids linked by peptide bonds. A peptide refers to a polypeptide of 2 amino acids to about 50 amino acids in length. In an aspect, a polypeptide is 2 to 50 amino acids long. In an aspect, a polypeptide is 10 to 50 amino acids long. In an aspect, a polypeptide is 15 to 45 amino acids long. In an aspect, a polypeptide is 20 to 40 amino acids long. In an aspect, a polypeptide is at least 30 amino acids long. In an aspect, a polypeptide is at least 40 amino acids long. In an aspect, a polypeptide is at least 50 amino acids long. In an aspect, a polypeptide is at least 60 amino acids long. In an aspect, a polypeptide is at least 70 amino acids long. In an aspect, a polypeptide is at least 80 amino acids long. In an aspect, a polypeptide is at least 90 amino acids long. In an aspect, a polypeptide is 40 to 100 amino acids long. In an aspect, a polypeptide is 50 to 90 amino acids long. In an aspect, a polypeptide is 60 to 80 amino acids long. In an aspect, a polypeptide is 50 to 70 amino acids long. In an aspect, a polypeptide is 60 to 90 amino acids long. In an aspect, a polypeptide is 40 to 80 amino acids long.

As used herein, “protein” refers to a biomolecule or macromolecule comprising one or more chains of amino acids linked by peptide bonds that has molecular weight greater than about 10,000 Daltons. In an aspect, a protein is a polypeptide. In an aspect, a protein comprises at least one polypeptide. In an aspect, a protein comprises two or more polypeptides. In an aspect, a protein is at least 90 amino acids long. In an aspect, a protein is about 90 amino acids to about 35,000 amino acids long. In an aspect, a protein is about 100 to about 2,000 amino acids long. In an aspect, a protein is about 200 to about 1,000 amino acids long. In an aspect, a protein is about 100 to about 900 amino acids long. In an aspect, a protein is about 150 to about 800 amino acids long. In an aspect, a protein is about 160 to about 700 amino acids long. In an aspect, a protein is about 200 to about 600 amino acids long.

As used herein, “small molecule” refers to a lower molecular weight (usually ≤1,000 Daltons) organic compound. In an aspect, a small molecule is capable of binding to a specific biological targe, and is capable of altering the activity or function of the target.

As used herein, “carbohydrate” refers to a compound having the general formula Cₓ(H2O)_{y}.

As used herein, “lipid” refers to a fatty acid or fatty acid derivative that is soluble in organic solvents but insoluble in water.

As used herein, “biopolymer” refers to a polymeric substance comprising a series of covalently-linked monomers that is biosynthesized by, or derived from, a living organism. Examples of biopolymers include polynucleotides, polypeptides, and polysaccharides.

As used herein, “linkage” refers to a connection between two chemical fragments. In an aspect, a linkage is a covalent bond. In an aspect, a linkage may comprise one or more chemical moieties. In an aspect, a linkage may comprise a phosphate, a phosphorothioate, a phosphoramidate, an amine, an amide, a triazole, an ether, and a thioether, and any combination thereof. In an aspect, a linkage is selected from the group consisting of a phosphate linkage, a phosphorothioate linkage, a phosphoramidate linkage, an amine linkage, an amide linkage, a triazole linkage, an ether linkage, and a thioether linkage.

As used herein, “ligation” refers to the covalent joining of two nucleic acid fragments through the action of an enzyme. In an aspect, the enzyme is a DNA ligase. In an aspect, the enzyme is an RNA ligase. In an aspect, the enzyme is T4 RNA Ligase I.

As used herein, “payload” refers to the molecule that is delivered to a cell. In an aspect, a payload is a polypeptide. In an aspect, a payload is a protein. In an aspect, a payload is a small molecule. In an aspect, a payload is an antibody. In an aspect, a payload is an enzyme. In an aspect, a payload is a bioconjugate. In an aspect, a payload is an antibody-drug conjugate (ADC).

As used herein, “targeted therapy” refers to a therapy where the therapeutic agent is delivered to a target area or cell. In an aspect, the target is a specific cell type. In an aspect, the target is a specific organ. In an aspect, the target is a specific anatomical location. In an aspect, the target is cells expressing specific cell surface receptors. In an aspect, the target is specific diseased cells.

As used herein, “enzyme replacement therapy” or “ERT” refers to a medical treatment whereby replacement enzymes are given to patients suffering from enzyme deficiencies or malfunction. Examples of such deficiencies or malfunction result in conditions that include, but are not limited to, Gaucher disease, Fabry disease, Pompe disease, glycogen storage disease type II, metachromatic leukodystrophy, gangliosidoses, Hunter syndrome, mucopolysaccharidoses, multiple sulfatase deficiency, corneal clouding, hepatosplenomegaly, Morquio syndrome, MPS disorders, mucolipidosis, neurologic manifestations, Sandhoff disease, sialidosis, lysosomal acid lipase deficiency, adenosine deaminase deficiency, and thrombotic thrombocytopenic purpura.

As used herein, “vaccine therapy” refers to therapy that uses one or more substances to stimulate the immune system to destroy a tumor or infectious microorganisms such as bacteria or viruses. In an aspect, vaccine therapy is cancer vaccine therapy.

As used herein, “expression” of a polypeptide refers to the production of the polypeptide by the process of translation. Without being bound by theory, during translation, a ribosome decodes the sequence in a messenger RNA (mRNA) and produces a polypeptide with an amino acid sequence that corresponds in accordance with the genetic code. Further, “co-expression” of two polypeptides refers to the expression of two different polypeptides within a single cell.

As used herein, “contacting” A to B refers to the act of bringing A and B physically together such that they are touching.

As used herein, a “therapeutic” agent refers to an agent that has properties of treating disease or disorders. In an aspect, a therapeutic agent may be a therapeutic polypeptide. In an aspect, a therapeutic agent may be a therapeutic enzyme. In an aspect, a therapeutic agent may be a therapeutic antibody. In an aspect, a therapeutic agent may be a therapeutic small molecule.

As used herein, “cancer” is a group of diseases involving abnormal cell growth with the potential to invade or spread to other parts of the body. Cancers may start in almost any organ or tissue in the body. As used herein, cancer also includes but is not limited to hyperplasia, dysplasia, and carcinoma in situ. In an aspect, a cancer is carcinoma. In an aspect, a cancer is sarcoma. In an aspect, a cancer is leukemia. In an aspect, a cancer is a lymphoma. In an aspect, a cancer is breast cancer. In an aspect, a cancer is lung cancer. In an aspect, a cancer is pancreatic cancer. In an aspect, a cancer is colorectal cancer.

As used herein, “obesity” is a disease or disorder characterized by excessive body fat which increases the risk of various health problems.

As used herein, “body mass index” or “BMI” is a measure of body fat based on height and weight. In an aspect, BMI is the body mass divided by the square of the body height, and is expressed in units of kg/m². A BMI of 25 to less than 30 is classified as overweight. A BMI of 30 to less than 35 is classified as class 1 obesity. A BMI of 35 to less than 40 is classified as class 2 obesity. A BMI of 40 or higher is classified as class 3 obesity, also referred to as severe obesity.

As used herein, “effective amount” or “therapeutically effective amount” refers to the amount of active pharmaceutical agent sufficient to effectuate a desired physiological outcome in an individual in need of the agent. The effective amount can vary among individuals depending on the health and physical condition of the individual to be treated, the taxonomic group of the individuals to be treated, the formulation of the composition, assessment of the individual's medical condition, and other relevant factors.

As used herein, “patient” or “subject” or “individual” refers to a human or non-human animal selected for treatment or therapy. In an aspect, the patient is a human.

As used herein, “pharmaceutical agent” refers to a substance that provides a therapeutic benefit when administered to an individual.

As used herein, “pharmaceutical composition” refers to a mixture of substances suitable for administering to an individual. For example, a pharmaceutical composition can comprise one or more active agents and a sterile aqueous solution.

As used herein, “pharmaceutically acceptable”, unless otherwise noted, is used to characterize a moiety (e.g., a salt, dosage form, or excipient) as being appropriate for use in accordance with sound medical judgment. In general, a pharmaceutically acceptable moiety has one or more benefits that outweigh any deleterious effect that the moiety may have. Deleterious effects may include, for example, excessive toxicity, irritation, allergic response, and other problems and complications.

As used herein, “formulation” refers to a medication in which different ingredients are combined, and which is administered in a specific form, e.g., by tablet or injection.

As used herein, “treat” or “treatment” or “treating” refers to administering a pharmaceutical composition to obtain beneficial or desired clinical results. In an aspect, the term “treat” or “treatment” means to administer the oligonucleotide bioconjugates, mRNA bioconjugates, or pharmaceutical formulations disclosed herein that partially or completely alleviate, ameliorate, relieve, inhibit, reduce severity of, and/or reduce incidence of one or more symptoms, features, or causes of the disease, condition, or disorder.

As used herein, “preventing” refers to administering a pharmaceutical composition prophylactically to stop the onset of disease. In an aspect, administering a pharmaceutical composition prophylactically stops the manifestation of clinical or subclinical symptoms thereof.

As used herein, “C₁-C₆” means a carbon group having 1, 2, 3, 4, 5, or 6 carbon atoms. As used herein, “C₁-C₂₀” means a carbon group having between 1 and 20 carbon atoms. As used herein, “C₃-C₇” means a carbon group having between 3 and 7 carbon atoms. As used herein, “C₃-C₂₀” means a carbon group having between 3 and 20 carbon atoms. As used herein, “C₄-C₂₀” means a carbon group having between 4 and 20 carbon atoms. As used herein, “C₇-C₂₀” means a carbon group having between 7 and 20 carbon atoms.

As used herein, “alkyl”, unless otherwise noted, includes both straight and branched chain alkyl groups and may be substituted or non-substituted. Alkyl groups include, but are not limited to, methyl, ethyl, n-propyl, iso-propyl, n-butyl, iso-butyl, sec-butyl, tert-butyl, and pentyl.

As used herein, “alkenyl”, unless otherwise noted, includes both straight and branched chain alkyl groups containing at least one double bond, and which may be substituted or non-substituted. Alkenyl groups include, but are not limited to, vinyl, allyl, propenyl, and butenyl.

As used herein, “alkynyl”, unless otherwise noted, includes both straight and branched chain alkyl groups containing at least one triple bond, and which may be substituted or non-substituted. Alkynyl groups include, but are not limited to, ethynyl, propynyl, butynyl, and propargyl.

As used herein, “cycloalkyl”, unless otherwise noted, includes cyclic alkyl groups and may be substituted or non-substituted. Cycloalkyl groups include, but are not limited to, cyclopropyl, cyclobutyl, cyclopentyl, and cyclohexyl.

As used herein, “alkyl-cycloalkyl”, unless otherwise noted, refers to groups having a combination of straight and/or branched chain alkyl segments, which may be substituted or non-substituted, and cyclic alkyl segments, which may be substituted or non-substituted. Alkyl-cycloalkyl groups include, but are not limited to, cyclopentylmethyl, cyclopentylethyl, cyclobutylmethyl, cyclobutylethyl, cyclopentylmethyl, cyclopentylethyl, cyclohexylmethyl, and cyclohexylethyl.

As used herein, “aryl”, unless otherwise noted, refers to a monocyclic or polycyclic aromatic ring system. Aryl groups include, but are not limited to, phenyl, naphthyl, anthryl, and biphenyl.

As used herein, “alkyl-aryl”, unless otherwise noted, refers to groups having a combination of straight and/or branched chain alkyl segments, which may be substituted or non-substituted, and at least one aryl group, which may be substituted or non-substituted.

As used herein, “heteroaryl”, unless otherwise noted, refers to an aromatic monocyclic or bicyclic group in which one or more ring atoms are nitrogen, oxygen, or sulfur, and the remaining ring atoms are carbon. Heteroaryl groups include, but are not limited to, pyrrolyl, furanyl, thienyl, oxazolyl, isoxazolyl, thiazolyl, isothiazolyl, imidazolyl, triazolyl, tetrazolyl, pyridinyl, pyrimidinyl, pyrazinyl, pyridazinyl, indoyl, benzofuranyl, benzothiophenyl, thiophenyl, benzimidazolyl, quinolinyl, isoquinolinyl, quinazolinyl, quinoxalinyl, and pyrazolyl.

As used herein “halogen” refers to toludide, chloride, boride, or iodide.

As used herein, “heterocycloalkyl” refers to a cycloalkyl group in which one or more of the ring methylene groups has been replaced with a group selected from —O—, —S—, —NH—, and —NR—, where R is an alkyl, cycloakyl, alkyl-cycloalkyl, or aryl group. In an aspect, the ring carbon atoms of the heterocycloalkyl are substituted. In an aspect, none of the ring carbon atoms of the heterocycloalkyl are substituted, indicating that the heterocycloalkyl group is unsubstituted.

As used herein, “alkyl-heteroaryl”, unless otherwise noted, refers to groups having a combination of straight and/or branched chain alkyl segments, which may be substituted or non-substituted, and at least one heteroaryl group, which may be substituted or non-substituted. Alkyl-heteroaryl groups include, but are not limited to, benzyl and homobenzyl.

As used herein, “acyl” refers to a group or radical of the form —C(═O)R, where R is any organic group.

As used herein, “polyamide” refers to a polymer comprising two or more amide linkages. In an aspect, a polyamide refers to a polymer with repeating units linked by amide bonds.

As used herein, a wavy line,

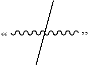

denotes a point of attachment of a substituent to another group.

As used herein, “co-administration” refers to administration of two or more agents to an individual. The two or more agents can be in a single pharmaceutical composition, or can be in separate pharmaceutical compositions. Each of the two or more agents can be administered through the same or different routes of administration. Co-administration encompasses simultaneous or sequential administration.

All publications, patents, and patent applications mentioned in this disclosure are herein incorporated by reference to the same extent as if each individual publication, patent, or patent application was specifically and individually indicated to be incorporated by reference.

### B. Oligonucleotide Bioconjugates and mRNA Bioconjugates

The present disclosure provides for, and includes, an oligonucleotide bioconjugate, or a pharmaceutically acceptable salt thereof, of formula (I)

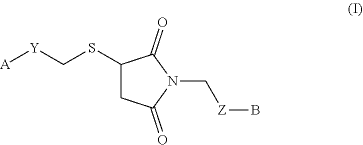

wherein
   A is an oligonucleotide having a 3′-end and a 5′-end;
   Y is a C₁-C₂₀ alkyl, wherein any one or more —CH₂— groups of the C₁-C₂₀ alkyl is each optionally replaced independently with —C(═O)—, —CF₂—, or a heteroatomic moiety selected from the group consisting of —O—, —S—, —NH—, and —N(C₁-C₆ alkyl)-, wherein any one or more —CH₃ groups of the C₁-C₂₀ alkyl is each optionally replaced independently with —CF₃, —CF₂H, —CH₂F, or a heteroatomic moiety selected from the group consisting of —NH(C₁-C₆ alkyl), —N(C₁-C₆ alkyl)₂, —O(C₁-C₆ alkyl), and —S(C₁-C₆ alkyl), and wherein any two heteroatomic moieties are separated from one another by at least two carbon atoms;
   Z is a C₁-C₂₀ alkyl, wherein any one or more —CH₂— groups of the C₁-C₂₀ alkyl is each optionally replaced independently with —C(═O)—, —CF₂—, or a heteroatomic moiety selected from the group consisting of —O—, —S—, —NH—, and —N(C₁-C₆ alkyl)-, wherein any one or more —CH₃ groups of the C₁-C₂₀ alkyl is each optionally replaced independently with —CF₃, —CF₂H, —CH₂F, or a heteroatomic moiety selected from the group consisting of —NH(C₁-C₆ alkyl), —N(C₁-C₆ alkyl)₂, —O(C₁-C₆ alkyl), and —S(C₁-C₆ alkyl), and wherein any two heteroatomic moieties are separated from one another by at least two carbon atoms; and
   B is an oligonucleotide, a polypeptide, a protein, a small molecule, a carbohydrate, a lipid, a polyethylene glycol (PEG) molecule, or a biopolymer,
wherein Y is covalently attached to the 3′-end of A through a first linkage.

The present disclosure provides for, and includes, an oligonucleotide bioconjugate, or a pharmaceutically acceptable salt thereof, of formula (II)

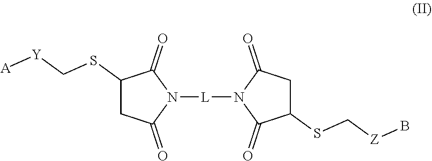

wherein
   A is an oligonucleotide having a 3′-end and a 5′-end;
   Y is a C₁-C₂₀ alkyl, wherein any one or more —CH₂— groups of the C₁-C₂₀ alkyl is each optionally replaced independently with —C(═O)—, —CF₂—, or a heteroatomic moiety selected from the group consisting of —O—, —S—, —NH—, and —N(C₁-C₆ alkyl)-, wherein any one or more —CH₃ groups of the C₁-C₂₀ alkyl is each optionally replaced independently with —CF₃, —CF₂H, —CH₂F, or a heteroatomic moiety selected from the group consisting of —NH(C₁-C₆ alkyl), —N(C₁-C₆ alkyl)₂, —O(C₁-C₆ alkyl), and —S(C₁-C₆ alkyl), and wherein any two heteroatomic moieties are separated from one another by at least two carbon atoms;
   L is a C₂-C₅₀ alkyl, C₂-C₅₀ alkenyl, C₂-C₅₀ alkynyl, C₃-C₈ cycloalkyl, aryl, heteroaryl, C₄-C₅₀ alkyl-cycloalkyl, C₇-C₅₀ alkyl-aryl, or C₆-C₅₀ alkyl-heteroaryl, wherein any one or more —CH₂— groups of L is each optionally replaced independently with —C(═O)—, —CF₂—, or a heteroatomic moiety selected from the group consisting of —O—, —S—, —NH—, and —N(C₁-C₆ alkyl)-, wherein any one or more —CH₃ groups of L is each optionally replaced independently with —CF₃, —CF₂H, —CH₂F, or a heteroatomic moiety selected from the group consisting of —NH(C₁-C₆ alkyl), —N(C₁-C₆alkyl)₂, —O(C₁-C₆ alkyl), and —S(C₁-C₆ alkyl), and wherein any two heteroatomic moieties are separated from one another by at least two carbon atoms;
   Z is a C₁-C₂₀ alkyl, wherein any one or more —CH₂— groups of the C₁-C₂₀ alkyl is each optionally replaced independently with —C(═O)—, —CF₂—, or a heteroatomic moiety selected from the group consisting of —O—, —S—, —NH—, and —N(C₁-C₆ alkyl)-, wherein any one or more —CH₃ groups of the C₁-C₂₀ alkyl is each optionally replaced independently with —CF₃, —CF₂H, —CH₂F, or a heteroatomic moiety selected from the group consisting of —NH(C₁-C₆ alkyl), —N(C₁-C₆ alkyl)₂, —O(C₁-C₆ alkyl), and —S(C₁-C₆ alkyl), and wherein any two heteroatomic moieties are separated from one another by at least two carbon atoms; and
   B is an oligonucleotide, a polypeptide, a protein, a small molecule, a lipid, a carbohydrate, a polyethylene glycol (PEG) molecule, or a biopolymer,
wherein Y is covalently attached to the 3′-end of A through a first linkage.

The present disclosure provides for, and includes, an oligonucleotide bioconjugate, or a pharmaceutically acceptable salt thereof, of formula (III)

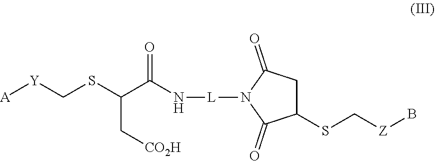

wherein
   A is an oligonucleotide having a 3′-end and a 5′-end;
   Y is a C₁-C₂₀ alkyl, wherein any one or more —CH₂— groups of the C₁-C₂₀ alkyl is each optionally replaced independently with —C(═O)—, —CF₂—, or a heteroatomic moiety selected from the group consisting of —O—, —S—, —NH—, and —N(C₁-C₆ alkyl)-, wherein any one or more —CH₃ groups of the C₁-C₂₀ alkyl is each optionally replaced independently with —CF₃, —CF₂H, —CH₂F, or a heteroatomic moiety selected from the group consisting of —NH(C₁-C₆ alkyl), —N(C₁-C₆ alkyl)₂, —O(C₁-C₆ alkyl), and —S(C₁-C₆ alkyl), and wherein any two heteroatomic moieties are separated from one another by at least two carbon atoms;
   L is a C₂-C₅₀ alkyl, C₂-C₅₀ alkenyl, C₂-C₅₀ alkynyl, C₃-C₈ cycloalkyl, aryl, heteroaryl, C₄-C₅₀ alkyl-cycloalkyl, C₇-C₅₀ alkyl-aryl, or C₆-C₅₀ alkyl-heteroaryl, wherein any one or more —CH₂— groups of L is each optionally replaced independently with —C(═O)—, —CF₂—, or a heteroatomic moiety selected from the group consisting of —O—, —S—, —NH—, and —N(C₁-C₆ alkyl)-, wherein any one or more —CH₃ groups of L is each optionally replaced independently with —CF₃, —CF₂H, —CH₂F, or a heteroatomic moiety selected from the group consisting of —NH(C₁-C₆ alkyl), —N(C₁-C₆alkyl)₂, —O(C₁-C₆ alkyl), and —S(C₁-C₆ alkyl), and wherein any two heteroatomic moieties are separated from one another by at least two carbon atoms;
   Z is a C₁-C₂₀ alkyl, wherein any one or more —CH₂— groups of the C₁-C₂₀ alkyl is each optionally replaced independently with —C(═O)—, —CF₂—, or a heteroatomic moiety selected from the group consisting of —O—, —S—, —NH—, and —N(C₁-C₆ alkyl)-, wherein any one or more —CH₃ groups of the C₁-C₂₀ alkyl is each optionally replaced independently with —CF₃, —CF₂H, —CH₂F, or a heteroatomic moiety selected from the group consisting of —NH(C₁-C₆ alkyl), —N(C₁-C₆ alkyl)₂, —O(C₁-C₆ alkyl), and —S(C₁-C₆ alkyl), and wherein any two heteroatomic moieties are separated from one another by at least two carbon atoms; and
   B is an oligonucleotide, a polypeptide, a protein, a small molecule, a lipid, a carbohydrate, a polyethylene glycol (PEG) molecule, or a biopolymer,
wherein Y is covalently attached to the 3′-end of A through a first linkage.

The present disclosure provides for, and includes, an oligonucleotide bioconjugate, or a pharmaceutically acceptable salt thereof, of formula (IV)

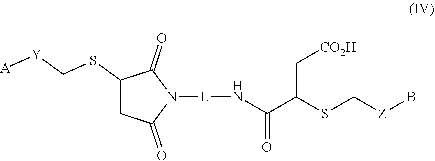

wherein
   A is an oligonucleotide having a 3′-end and a 5′-end;
   Y is a C₁-C₂₀ alkyl, wherein any one or more —CH₂— groups of the C₁-C₂₀ alkyl is each optionally replaced independently with —C(═O)—, —CF₂—, or a heteroatomic moiety selected from the group consisting of —O—, —S—, —NH—, and —N(C₁-C₆ alkyl)-, wherein any one or more —CH₃ groups of the C₁-C₂₀ alkyl is each optionally replaced independently with —CF₃, —CF₂H, —CH₂F, or a heteroatomic moiety selected from the group consisting of —NH(C₁-C₆ alkyl), —N(C₁-C₆ alkyl)₂, —O(C₁-C₆ alkyl), and —S(C₁-C₆ alkyl), and wherein any two heteroatomic moieties are separated from one another by at least two carbon atoms;
   L is a C₂-C₅₀ alkyl, C₂-C₅₀ alkenyl, C₂-C₅₀ alkynyl, C₃-C₈ cycloalkyl, aryl, heteroaryl, C₄-C₅₀ alkyl-cycloalkyl, C₇-C₅₀ alkyl-aryl, or C₆-C₅₀ alkyl-heteroaryl, wherein any one or more —CH₂— groups of L is each optionally replaced independently with —C(═O)—, —CF₂—, or a heteroatomic moiety selected from the group consisting of —O—, —S—, —NH—, and —N(C₁-C₆ alkyl)-, wherein any one or more —CH₃ groups of L is each optionally replaced independently with —CF₃, —CF₂H, —CH₂F, or a heteroatomic moiety selected from the group consisting of —NH(C₁-C₆ alkyl), —N(C₁-C₆alkyl)₂, —O(C₁-C₆ alkyl), and —S(C₁-C₆ alkyl), and wherein any two heteroatomic moieties are separated from one another by at least two carbon atoms;
   Z is a C₁-C₂₀ alkyl, wherein any one or more —CH₂— groups of the C₁-C₂₀ alkyl is each optionally replaced independently with —C(═O)—, —CF₂—, or a heteroatomic moiety selected from the group consisting of —O—, —S—, —NH—, and —N(C₁-C₆ alkyl)-, wherein any one or more —CH₃ groups of the C₁-C₂₀ alkyl is each optionally replaced independently with —CF₃, —CF₂H, —CH₂F, or a heteroatomic moiety selected from the group consisting of —NH(C₁-C₆ alkyl), —N(C₁-C₆ alkyl)₂, —O(C₁-C₆ alkyl), and —S(C₁-C₆ alkyl), and wherein any two heteroatomic moieties are separated from one another by at least two carbon atoms; and
   B is an oligonucleotide, a polypeptide, a protein, a small molecule, a lipid, a carbohydrate, a polyethylene glycol (PEG) molecule, or a biopolymer,
wherein Y is covalently attached to the 3′-end of A through a first linkage.

The present disclosure provides for, and includes, an oligonucleotide bioconjugate, or a pharmaceutically acceptable salt thereof, of formula (V)

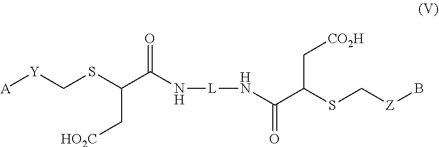

wherein
   A is an oligonucleotide having a 3′-end and a 5′-end;
   Y is a C₁-C₂₀ alkyl, wherein any one or more —CH₂— groups of the C₁-C₂₀ alkyl is each optionally replaced independently with —C(═O)—, —CF₂—, or a heteroatomic moiety selected from the group consisting of —O—, —S—, —NH—, and —N(C₁-C₆ alkyl)-, wherein any one or more —CH₃ groups of the C₁-C₂₀ alkyl is each optionally replaced independently with —CF₃, —CF₂H, —CH₂F, or a heteroatomic moiety selected from the group consisting of —NH(C₁-C₆ alkyl), —N(C₁-C₆ alkyl)₂, —O(C₁-C₆ alkyl), and —S(C₁-C₆ alkyl), and wherein any two heteroatomic moieties are separated from one another by at least two carbon atoms;
   L is a C₂-C₅₀ alkyl, C₂-C₅₀ alkenyl, C₂-C₅₀ alkynyl, C₃-C₈ cycloalkyl, aryl, heteroaryl, C₄-C₅₀ alkyl-cycloalkyl, C₇-C₅₀ alkyl-aryl, or C₆-C₅₀ alkyl-heteroaryl, wherein any one or more —CH₂— groups of the L is each optionally replaced independently with —C(═O)—, —CF₂—, or a heteroatomic moiety selected from the group consisting of —O—, —S—, —NH—, and —N(C₁-C₆ alkyl)-, wherein any one or more —CH₃ groups of L is each optionally replaced independently with —CF₃, —CF₂H, —CH₂F, or a heteroatomic moiety selected from the group consisting of —NH(C₁-C₆ alkyl), —N(C₁-C₆ alkyl)₂, —O(C₁-C₆ alkyl), and —S(C₁-C₆ alkyl), and wherein any two heteroatomic moieties are separated from one another by at least two carbon atoms;
   Z is a C₁-C₂₀ alkyl, wherein any one or more —CH₂— groups of the C₁-C₂₀ alkyl is each optionally replaced independently with —C(═O)—, —CF₂—, or a heteroatomic moiety selected from the group consisting of —O—, —S—, —NH—, and —N(C₁-C₆ alkyl)-, wherein any one or more —CH₃ groups of the C₁-C₂₀ alkyl is each optionally replaced independently with —CF₃, —CF₂H, —CH₂F, or a heteroatomic moiety selected from the group consisting of —NH(C₁-C₆ alkyl), —N(C₁-C₆ alkyl)₂, —O(C₁-C₆ alkyl), and —S(C₁-C₆ alkyl), and wherein any two heteroatomic moieties are separated from one another by at least two carbon atoms; and
   B is an oligonucleotide, a polypeptide, a protein, a small molecule, a lipid, a carbohydrate, a polyethylene glycol (PEG) molecule, or a biopolymer,
wherein Y is covalently attached to the 3′-end of A through a first linkage.

### 1. First Linkage

In an aspect, a first linkage covalently attaching Y to the 3′-end of A is selected from the group consisting of a phosphate linkage, a phosphorothioate linkage, a phosphoramidate linkage, an amine linkage, an amide linkage, a triazole linkage, an ether linkage, and a thioether linkage.

In an aspect, a first linkage covalently attaching Y to the 3′-end of A is a phosphate linkage. An example of the covalent attachment of a nucleotide located at the 3′-end of A through a phosphate linkage to Y is shown below in formula (A)

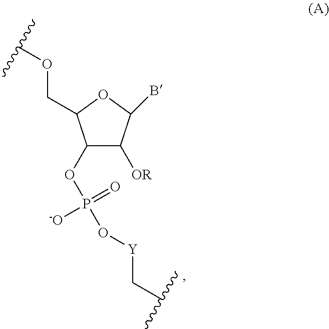

where B′ is a natural or non-natural nucleobase, and R is H, an alcohol protecting group, or a C₁-C₆alkyl, where any one or more —CH₂— groups of the C₁-C₆ alkyl is each optionally replaced independently with —CHF—, —CF₂—, or a heteroatomic moiety selected from the group consisting of —O— and —S—, and where any two heteroatomic moieties are separated from one another by at least two carbon atoms. In an aspect, a nucleotide located at the 3′-end of A comprises the phosphate linkage which covalently links A to Y. In an aspect, A comprises the phosphate linkage which covalently links A to Y. In an aspect, the phosphate linkage between Y and A comprises a phosphate attached to the 3′-oxygen atom of a nucleotide located at the 3′-end of A. In an aspect, the oligonucleotide bioconjugate of formula (I) comprises formula (A). In an aspect, the oligonucleotide bioconjugate of formula (II) comprises formula (A). In an aspect, the oligonucleotide bioconjugate of formula (III) comprises formula (A). In an aspect, the oligonucleotide bioconjugate of formula (IV) comprises formula (A). In an aspect, the oligonucleotide bioconjugate of formula (V) comprises formula (A).

In an aspect, a first linkage covalently attaching Y to the 3′-end of A is a phosphorothioate linkage. An example of the covalent attachment of a nucleotide located at the 3′-end of A through a phosphorothioate linkage to Y is shown below in formula (B)

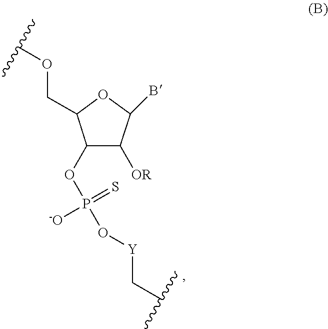

where B′ is a natural or non-natural nucleobase, and R is H, an alcohol protecting group, or a C₁-C₆ alkyl, where any one or more —CH₂— groups of the C₁-C₆ alkyl is each optionally replaced independently with —CHF—, —CF₂—, or a heteroatomic moiety selected from the group consisting of —O— and —S—, and where any two heteroatomic moieties are separated from one another by at least two carbon atoms. In an aspect, a nucleotide located at the 3′-end of A comprises the phosphorothioate linkage which covalently links A to Y. In an aspect, A comprises the phosphorothioate linkage which covalently links A to Y. In an aspect, the phosphorothioate linkage between Y and A comprises a phosphorothioate attached to the 3′-oxygen atom of a nucleotide located at the 3′-end of A. In an aspect, the oligonucleotide bioconjugate of formula (I) comprises formula (B). In an aspect, the oligonucleotide bioconjugate of formula (II) comprises formula (B). In an aspect, the oligonucleotide bioconjugate of formula (III) comprises formula (B). In an aspect, the oligonucleotide bioconjugate of formula (IV) comprises formula (B). In an aspect, the oligonucleotide bioconjugate of formula (V) comprises formula (B).

In an aspect, a first linkage covalently attaching Y to the 3′-end of A is a phosphoramidate linkage. An example of the covalent attachment of a nucleotide located at the 3′-end of A through a phosphoramidate linkage to Y is shown below in formula (C)

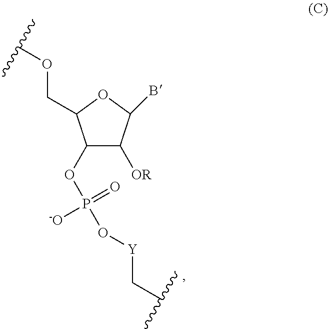

where B′ is a natural or non-natural nucleobase, and R is H, an alcohol protecting group, or a C₁-C₆alkyl, where any one or more —CH₂— groups of the C₁-C₆ alkyl is each optionally replaced independently with —CHF—, —CF₂—, or a heteroatomic moiety selected from the group consisting of —O— and —S—, and where any two heteroatomic moieties are separated from one another by at least two carbon atoms. In an aspect, a nucleotide located at the 3′-end of A comprises the phosphoramidate linkage which covalently links A to Y. In an aspect, A comprises the phosphoramidate linkage which covalently links A to Y. In an aspect, the phosphoramidate linkage between Y and A comprises a phosphoramidate attached to the 3′-oxygen atom of a nucleotide located at the 3′-end of A. In an aspect, the oligonucleotide bioconjugate of formula (I) comprises formula (C). In an aspect, the oligonucleotide bioconjugate of formula (II) comprises formula (C). In an aspect, the oligonucleotide bioconjugate of formula (III) comprises formula (C). In an aspect, the oligonucleotide bioconjugate of formula (IV) comprises formula (C). In an aspect, the oligonucleotide bioconjugate of formula (V) comprises formula (C).

In an aspect, a first linkage covalently attaching Y to the 3′-end of A is an amine linkage. An example of the covalent attachment of a nucleotide located at the 3′-end of A through an amine linkage to Y is shown below in formula (D)

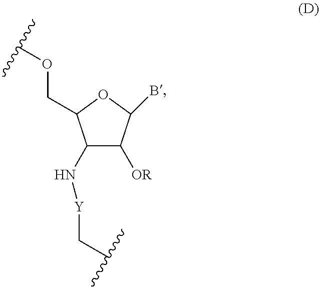

where B′ is a natural or non-natural nucleobase, and R is H, an alcohol protecting group, or a C₁-C₆alkyl, where any one or more —CH₂— groups of the C₁-C₆ alkyl is each optionally replaced independently with —CHF—, —CF₂—, or a heteroatomic moiety selected from the group consisting of —O— and —S—, and where any two heteroatomic moieties are separated from one another by at least two carbon atoms. In an aspect, a nucleotide located at the 3′-end of A comprises the amine linkage which covalently links A to Y. In an aspect, A comprises the amine linkage which covalently links A to Y. In an aspect, the amine linkage between Y and A comprises an amine attached to the 3′-position of a nucleotide located at the 3′-end of A. In an aspect, the oligonucleotide bioconjugate of formula (I) comprises formula (D). In an aspect, the oligonucleotide bioconjugate of formula (II) comprises formula (D). In an aspect, the oligonucleotide bioconjugate of formula (III) comprises formula (D). In an aspect, the oligonucleotide bioconjugate of formula (IV) comprises formula (D). In an aspect, the oligonucleotide bioconjugate of formula (V) comprises formula (D).

In an aspect, a first linkage covalently attaching Y to the 3′-end of A is an amide linkage. An example of the covalent attachment of a nucleotide located at the 3′-end of A through an amide linkage to Y is shown below in formula (E)

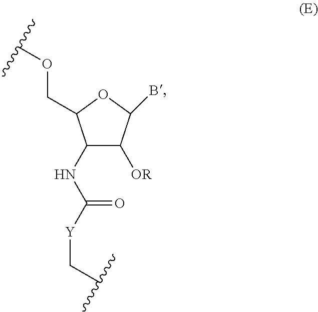

where B′ is a natural or non-natural nucleobase, and R is H or a C₁-C₆ alkyl, where any one or more —CH₂— groups of the C₁-C₆ alkyl is each optionally replaced independently with —CHF—, —CF₂—, or a heteroatomic moiety selected from the group consisting of —O— and —S—, and where any two heteroatomic moieties are separated from one another by at least two carbon atoms. In an aspect, a nucleotide located at the 3′-end of A comprises the amide linkage which covalently links A to Y. In an aspect, the final nucleotide located at the 3′-end of A comprises the NH of the amide linkage which covalently links A to Y. In an aspect, A comprises the amide group which covalently links A to Y. In an aspect, A comprises the NH of the amide group which covalently links A to Y. In an aspect, the amide linkage between Y and A comprises an amide attached to the 3′-position of a nucleotide located at the 3′-end of A. In an aspect, the oligonucleotide bioconjugate of formula (I) comprises formula (E). In an aspect, the oligonucleotide bioconjugate of formula (II) comprises formula (E). In an aspect, the oligonucleotide bioconjugate of formula (III) comprises formula (E). In an aspect, the oligonucleotide bioconjugate of formula (IV) comprises formula (E). In an aspect, the oligonucleotide bioconjugate of formula (V) comprises formula (E).

In an aspect, a first linkage covalently attaching Y to the 3′-end of A is a triazole linkage. Examples of the covalent attachment of a nucleotide located at the 3′-end of A through a triazole linkage to Y are shown below in formula (F) and formula (G)

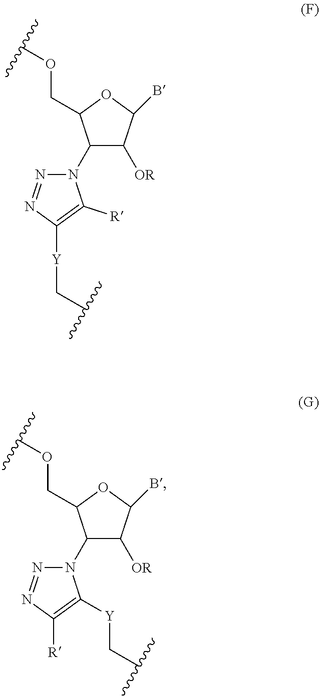

where B′ is a natural or non-natural nucleobase, R is H, an alcohol protecting group, or a C₁-C₆ alkyl, where any one or more —CH₂— groups of the C₁-C₆ alkyl is each optionally replaced independently with —CHF—, —CF₂—, or a heteroatomic moiety selected from the group consisting of —O— and —S—, and where any two heteroatomic moieties are separated from one another by at least two carbon atoms, and R′ is selected from the group consisting of H, C₁-C₁₂ alkyl, C₁-C₁₂ alkenyl, C₁-C₁₂ alkynyl, and C₁-C₁₂ cycloalkyl. In an aspect, a nucleotide located at the 3′-end of A comprises the triazole linkage which covalently links A to Y. In an aspect, A comprises the triazole linkage which covalently links A to Y. In an aspect, the triazole linkage between Y and A comprises a triazole attached to the 3′-position of a nucleotide located at the 3′-end of A. In an aspect, the oligonucleotide bioconjugate of formula (I) comprises formula (F) or formula (G). In an aspect, the oligonucleotide bioconjugate of formula (II) comprises formula (F) or formula (G). In an aspect, the oligonucleotide bioconjugate of formula (III) comprises formula (F) or formula (G). In an aspect, the oligonucleotide bioconjugate of formula (IV) comprises formula (F) or formula (G). In an aspect, the oligonucleotide bioconjugate of formula (V) comprises formula (F) or formula (G).

In an aspect, a first linkage covalently attaching Y to the 3′-end of A is an ether linkage. An example of the covalent attachment of a nucleotide located at the 3′-end of A through an ether linkage to Y is shown below in formula (H)

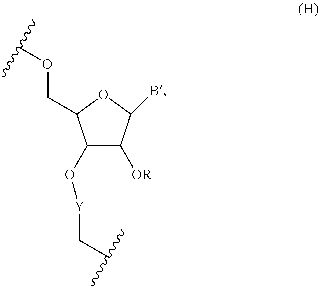

where B′ is a natural or non-natural nucleobase, and R is H, an alcohol protecting group, or a C₁-C₆ alkyl, where any one or more —CH₂— groups of the C₁-C₆ alkyl is each optionally replaced independently with —CHF—, —CF₂—, or a heteroatomic moiety selected from the group consisting of —O— and —S—, and where any two heteroatomic moieties are separated from one another by at least two carbon atoms. In an aspect, a nucleotide located at the 3′-end of A comprises the ether linkage which covalently links A to Y. In an aspect, A comprises the ether linkage which covalently links A to Y. In an aspect, the ether linkage between Y and A comprises a bond to the 3′-oxygen atom of a nucleotide located at the 3′-end of A. In an aspect, the oligonucleotide bioconjugate of formula (I) comprises formula (H). In an aspect, the oligonucleotide bioconjugate of formula (II) comprises formula (H). In an aspect, the oligonucleotide bioconjugate of formula (III) comprises formula (H). In an aspect, the oligonucleotide bioconjugate of formula (IV) comprises formula (H). In an aspect, the oligonucleotide bioconjugate of formula (V) comprises formula (H).

In an aspect, a first linkage covalently attaching Y to the 3′-end of A is a thioether linkage. An example of the covalent attachment of a nucleotide located at the 3′-end of A through a thioether linkage to Y is shown below in formula (J)

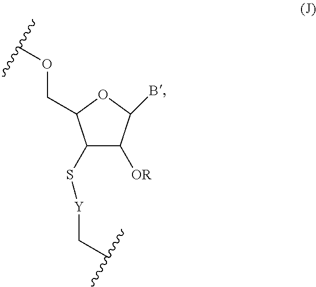

where B′ is a natural or non-natural nucleobase, and R is H, an alcohol protecting group, or a C₁-C₆alkyl, where any one or more —CH₂— groups of the C₁-C₆ alkyl is each optionally replaced independently with —CHF—, —CF₂—, or a heteroatomic moiety selected from the group consisting of —O— and —S—, and where any two heteroatomic moieties are separated from one another by at least two carbon atoms. In an aspect, a nucleotide located at the 3′-end of A comprises the thioether linkage which covalently links A to Y. In an aspect, A comprises the thioether linkage which covalently links A to Y. In an aspect, the thioether linkage between Y and A comprises a thioether attached to the 3′-position of a nucleotide located at the 3′-end of A. In an aspect, the oligonucleotide bioconjugate of formula (I) comprises formula (J). In an aspect, the oligonucleotide bioconjugate of formula (II) comprises formula (J). In an aspect, the oligonucleotide bioconjugate of formula (III) comprises formula (J). In an aspect, the oligonucleotide bioconjugate of formula (IV) comprises formula (J). In an aspect, the oligonucleotide bioconjugate of formula (V) comprises formula (J).

### 2. Cargo (B)

In an aspect, B is an oligonucleotide. In an aspect, B is a polypeptide. In an aspect, B is a protein. In an aspect, B is an antibody. In an aspect, B is a small molecule. In an aspect, B is a carbohydrate. In an aspect, B is cholesterol. In an aspect, B is a lipid. In an aspect, B is a PEG molecule. In an aspect, the PEG molecule is selected from the group consisting of PEG₄₀₀, PEG₁₅₀₀, PEG₂₀₀₀, PEG₃₃₅₀, PEG₄₀₀₀, PEG₆₀₀₀, PEG₈₀₀₀, PEG₁₀₀₀₀, PEG₁₅₀₀₀, PEG₂₀₀₀₀, and PEG₄₀₀₀₀. In an aspect, the PEG molecule is PEG₁₅₀₀. In an aspect, the PEG molecule is PEG₁₀₀₀₀. In an aspect, the PEG molecule is PEG₂₀₀₀₀. In an aspect, B is a biopolymer. In an aspect, the biopolymer is selected from the group consisting of proline/alanine/serine (PAS), XTEN, polysarcosine (pSar), polysaccharide, polyvinylpyrrolidone (PVP), polyglutamic acid (PGA), poly(hydroxyethyl-1-asparagine) (PHEA), poly(hydroxyethyl-1-glutamine) (PHEG), and poly(thioglycidyl glycerol) (PTTG), In an aspect, the biopolymer is PAS. In an aspect, the biopolymer is XTEN. In an aspect, the biopolymer is pSar. In an aspect, the biopolymer is PVP. In an aspect, the biopolymer is PGA. In an aspect, the biopolymer is PHEA. In an aspect, the biopolymer is PHEG. In an aspect, the biopolymer is PTTG.

In an aspect, B encodes an antibody. In aspect, B encodes the light chain of an antibody. In aspect, B encodes the heavy chain of an antibody. In an aspect, B encodes an antibody selected from the group consisting of adalimumab, dupilumab, trastuzumab, and pembrolizumab. In an aspect, B comprises a sequence selected from the group consisting of SEQ ID NOs: 108, 110, 112, 114, 116, and 118. In an aspect, B encodes an amino acid sequence selected from the group consisting of SEQ ID Nos: 109, 111, 113, 115, 117, and 119.

In an aspect, B encodes a peptide. In an aspect, B encodes a peptide selected from the group consisting of exenatide, GLP-1, gastric inhibitory peptide, and teriparatide. In an aspect, B comprises a sequence selected from the group consisting of SEQ ID NOs: 120, 122, 124, and 126. In an aspect, B encodes an amino acid sequence selected from the group consisting of SEQ ID Nos: 121, 123, 125, and 127.

a. Cell Penetrating Peptides

In an aspect, oligonucleotide bioconjugates can include polypeptides or proteins that aid in delivery of the oligonucleotide bioconjugates described herein into cells. Without being bound by theory, polypeptides or proteins with cell penetrating properties can increase cellular uptake and endosomal escape of the oligonucleotide bioconjugates. In an aspect, oligonucleotide bioconjugates described herein comprise a polypeptide or protein with cell penetrating properties. In an aspect, B is a cell penetrating polypeptide. Examples of cell penetrating peptides are described in the literature, for example, Qian et al., “Early Endosomal Escape of a Cyclic Cell-Penetrating Peptide Allows Effective Cytosolic Cargo Delivery,” Biochemistry 53(24): 4034-4046 (2014); Kalbenkova et al., “Chemistry of Peptide-Oligonucleotide Conjugates: A Review,” Molecules 26(17): 5420 (2021); Klipp et al., “Get out or die trying: Peptide- and protein-based endosomal escape of RNA therapeutics,” Adv. Drug Delivery Rev. 200:115047 (2023); Yokoo et al., “Cell-Penetrating Peptides: Emerging Tools for mRNA Delivery,” Pharmaceutics 14(1):78 (2021); the disclosures of which are incorporated by reference herein in their entireties for all purposes. In an aspect, B is a selected from the group consisting of a polycationic cell penetrating polypeptide, an amphipathic cell penetrating polypeptide, and a hydrophobic cell penetrating polypeptide.

In an aspect, B is a polycationic cell penetrating polypeptide. Without being bound by theory, electrostatic interactions between cationic cell penetrating polypeptides and negatively charged cytoplasmic membrane result in cellular internalization. In an aspect, the polycationic cell penetrating polypeptide is selected from the group consisting of the Tat peptide (RKKRRQRRR; SEQ ID NO: 1), penetratin (RQIKIWFQNRRMKWKK; SEQ ID NO: 2), polyarginine (RRRRRRRR; SEQ ID NO: 3), and cFΦR4 (cyclo(FΦRRRRQ); SEQ ID NO: 4).

Without being bound by theory, the Tat peptide, derived from the HIV-1 trans-activator of transcription protein at positions 48-60, internalizes into mammalian cells and activates viral replication. In an aspect, B is a fragment of the Tat peptide. In an aspect, B comprises the Tat peptide. In an aspect, B is the Tat peptide (RKKRRQRRR; SEQ ID NO: 1).

Without being bound by theory, penetratin is a short, positively charged peptide sequence derived from the Antennapedia protein that can effectively deliver molecules across cell membranes. In an aspect, B is penetratin (RQIKIWFQNRRMKWKK; SEQ ID NO: 2).

Without being bound by theory, polyarginine peptides bind to phospholipid phosphate groups, which destabilizes the membrane and creates a pore that allows peptide entry. In an aspect, B is polyarginine. In an aspect, B is arginine-8 (RRRRRRRR; SEQ ID NO: 3). In an aspect, B is an 9-mer of arginine. In an aspect, B is an 11-mer of arginine. In an aspect, B is a fatty acid modified polyarginine. In an aspect, B is cholesteryl oligoarginine.

Without being bound by theory, cyclization of certain arginine-rich CPPs enhances their cellular uptake compared to non-cyclized polyarginines. In an aspect, B is cyclo (FΦRRRRQ; SEQ ID NO: 4) (cFΦR4, where Φ is 1-2-naphthylalanine). In an aspect, B is cFΦR4.

In an aspect, B is an amphipathic cell penetrating peptide. In an aspect, the amphipathic cell penetrating polypeptide is selected from the group consisting of MPG (KETWWETWWTEWSQPKKRK; SEQ ID NO: 5), Pep-1 (GLAFLGFLGAAGSTMGAWSQPKKKRK; SEQ ID NO: 6), ARF (1-22) (MVRRFLVTLRIRRACGPPRVR; SEQ ID NO: 7), BPrPp (1-28) (MVKSKIGSWILVLFVAMWSDVGLCKKRPKP; SEQ ID NO: 8), MAP (KLALKALKALKAALKLA; SEQ ID NO: 9), transportan (GWTLNSAGYLLGKINLKALAALAKKIL; SEQ ID NO: 10), TP-10 (AGYLLGKINLKALAALAKKIL; SEQ ID NO: 11), CADY (GLWRALWRLLRSLWRLLWRA; SEQ ID NO: 12), RICK (KWLLRWLSRLLRWLARWLG; SEQ ID NO: 13), 599 (GLFEAIEGFIENGWEGMIDGWYGGGGRRRRRRRRRK; SEQ ID NO: 14), p28 (LSTAADMQGVVTDGMASGLDKDYLKPD; SEQ ID NO: 15), Bac7 (RRIRPRPPRLPRPRPRPLPFP; SEQ ID NO: 16), a proline-rich polypeptide (e.g., (PPR)n or (PRR)n (n=3-6)), and melittin (GIGAVLKVLTTGLPALISWIKRKRQQ; SEQ ID NO: 17). In an aspect, B is MPG. In an aspect, B is Pep-1. In an aspect, B is ARF (1-22). In an aspect, B is BPrPp (1-28). In an aspect, B is MAP. In an aspect, B is transportan. In an aspect, B is TP-10. In an aspect, B is CADY. In an aspect, B is RICK. In an aspect, B is 599. In an aspect, B is p28. In an aspect, B is Bac7. In an aspect, B is a proline-rich polypeptide. In an aspect, B is melittin.

In an aspect, B is a hydrophobic cell penetrating polypeptide. Without being bound by theory, hydrophobic cell penetrating peptides may reduce the risk of cell cytotoxicity and may accumulate less in organs compared to cationic cell penetrating peptides. In an aspect, the hydrophobic cell penetrating polypeptide is selected from the group consisting of C₁₀₅Y (CSIPPEVKFNKPFVYLI; SEQ ID NO: 18), Pep-7 (SDLWEMMMVSLACQ; SEQ ID NO: 19), P4 (LGAQSNF; SEQ ID NO: 20), Pepti (PLILLRLLRGQF; SEQ ID NO: 21), PTD1 (PFVYLI; SEQ ID NO: 22), and PTD2 (WSYGLRPG; SEQ ID NO: 23).

In an aspect, B is a polypeptide that induces endosomal membrane disruption. In an aspect, B is a polypeptide that enhances cellular uptake. In an aspect, B is a polypeptide that both induces endosomal membrane disruption and enhances cellular uptake. In an aspect, B is a cell penetrating polypeptide. In an aspect, B is selected from the group consisting of RALA (WEARLARALARALARHLARALARALRACEA; SEQ ID NO: 24), Pepfect 14 (PF14) (Stearyl-AGYLLGKLLOOLAAAALOOLL; SEQ ID NO: 25), and KALA (WEAKLAKALAKALAKHLAKALAKALKA; SEQ ID NO: 26). In an aspect, B is RALA. In an aspect, B is PF14, which has a sequence of Stearyl-AGYLLGKLLOOLAAAALOOLL where O is poly-L ornithine. In an aspect, B is KALA. In an aspect, B is formulated as a non-covalent complex with an mRNA. In an aspect, B is formulated as a covalent complex with an mRNA. In an aspect, B is formulated as a lipoplex comprising lipid nanoparticles (LNPs) attached to or complexed with a non-covalent complex of B and an mRNA. In an aspect, B is formulated as a lipoplex comprising lipid nanoparticles (LNPs) attached to or complexed with a covalent complex of B and an mRNA.

In an aspect, B is a polypeptide that modulates endocytotic pathways in dendritic cells (DCs). In an aspect, B is a cell penetrating polypeptide. In an aspect, B is selected from the group consisting of GALA (WEAALAEALAEALAEHLAEALAEALEALAA; SEQ ID NO: 27), LEDE (IGKEFKRIVERIKRFLRELVRPLR; SEQ ID NO: 28), LAH4-L1 (KKALLAHALHLLALLALHLAHALKKA; SEQ ID NO: 29), LAH4 (KKALLALALHHLAHLALHLALALKKA; SEQ ID NO: 30), and RALA. In an aspect, B is GALA. In an aspect, B is LEDE. In an aspect, B is LAH4-L1. In an aspect, B is RALA. In an aspect, B is formulated as a polyplex comprising polymers or nanoparticles coated with pegylated B. In an aspect, B is formulated as a nanoparticle complex comprising nanoparticles coated with a non-covalent complex of B and an mRNA. In an aspect, B is formulated as a nanoparticle complex comprising nanoparticles coated with a covalent complex of B and an mRNA.

In an aspect, B is a polypeptide that is a lung surfactant mimetic. In an aspect, B is a cell penetrating polypeptide. In an aspect, B is KL4 (KLLLLKLLLLKLLLLKLLLLK; SEQ ID NO: 31). In an aspect, B is formulated as a non-covalent complex of pegylated B with an mRNA. In an aspect, B is formulated as a covalent complex of pegylated B with an mRNA.

In an aspect, B is a peptide that is provides intracellular mRNA protection. In an aspect, B is a cell penetrating peptide. In an aspect, B is a peptide comprising oligoarginine and an α-aminoisobutyric acid (Aib) (OligoArg-Aib). In an aspect, B is OligoArg-Aib (RRXRRXRRXRRXRRX, where X represents Aib; SEQ ID NO: 32) or OligoArg (RRRRRRRRR; SEQ ID NO: 33). In an aspect, B is OligoArg-Aib. In an aspect, B is OligoArg. In an aspect, B is formulated as a non-covalent complex of pegylated B with an mRNA. In an aspect, B is formulated as a covalent complex of pegylated B with an mRNA.

In an aspect, B is a signal polypeptide for use intracellular localization, intracellular targeting, or both, as described in, for example, O'Neill et al., “Protein-Specific Signal Peptides for Mammalian Vector Engineering,” ACS Synthetic Biology 12(8): 2239-2352 (2023), the disclosure of which is incorporated by reference herein in its entirety for all purposes. In an aspect, the signal polypeptide is a metalloproteinase inhibitor 1 (TIMP1)N-terminal signal polypeptide, for example, SEQ ID NO: 34 (MAPFASLASGILLLLSLITSSKA). In an aspect, the signal polypeptide is a chronodroitin sulphate proteoglycan 4 (CSPG4)N-terminal signal polypeptide, for example, SEQ ID NO: 35 (MLLGPGHTLSAPALALAVTLTLLVRSASP). In an aspect, the signal polypeptide is a calreticulin (CALR)N-terminal signal peptide (CSPG4)N-terminal signal polypeptide, for example, SEQ ID NO: 36 (MLLSVPLLLGLLGLAAA). In an aspect, the signal polypeptide is a Dickkopf-related protein 3 (DKK3)N-terminal signal polypeptide, for example, SEQ ID NO: 37 (MQELRGILLCLLLAAAVPTTP). In an aspect, the signal polypeptide is a 60S acidic ribosomal protein P2 (RPLP2)N-terminus, for example, SEQ ID NO: 38 (MRYVASYLLAALGGNS). In an aspect, the signal polypeptide is a complement C is (CIS)N-terminal signal polypeptide, for example, SEQ ID NO: 39 (MGKSPEAWCIVLFSVLASFSA). In an aspect, the signal polypeptide is a cathepsin Z (CTSZ) N-terminal signal polypeptide, for example, SEQ ID NO: 40 (MASSGSVQQPRLVLLMLVLAGAARA). In an aspect, the signal polypeptide is a nucleobinin-2 (NUCB2)N-terminal signal polypeptide, for example, SEQ ID NO: 41 (MRWKIIQLQYCFLLVPCMLTALEA). In an aspect, the signal polypeptide is a protein disulphide-isomerase (PDIA1)N-terminal signal polypeptide, for example, SEQ ID NO: 42 (MLSRSLLCLALAWVARVGA). In an aspect, the signal polypeptide is a protein disulphide-isomerase A3 (PDIA3)N-terminal signal polypeptide, for example, SEQ ID NO: 43 (MRFSCLALLPGVALLLASARLAAA). In an aspect, the signal polypeptide is an endoplasmin (HSP90B1)N-terminal signal polypeptide, for example, SEQ ID NO: 44 (MRVLWVLGLCCVLLTFGFVRA). In an aspect, the signal polypeptide is a BiP (HSPA5)N-terminal signal polypeptide, for example, SEQ ID NO: 45 (MKFPMVAAALLLLCAVRA). In an aspect, the signal polypeptide is a Serpinh1 N-terminal signal polypeptide, for example, SEQ ID NO: 46 (MRSLLLASFCLLAVALA). In an aspect, the signal polypeptide is a clusterin (CLU)N-terminal signal polypeptide, for example, SEQ ID NO: 47 (MKILLLCVGLLLTWDNGMVLG). In an aspect, the signal polypeptide is a peptidylprolyl isomerase B (PPIB)N-terminal signal polypeptide, for example, SEQ ID NO: 48 (MLRISGRNMKVLFAAALIVGSVVFLLLPGPSVA). In an aspect, the signal polypeptide is a hypoxia upregulated protein 1 (HYOU1)N-terminal signal polypeptide, for example, SEQ ID NO: 49 (MAATVRRQRPRRLLCWTLVAVLLADLLALS). In an aspect, the signal polypeptide is a dolichyl-diphosphooligosaccharide protein glycotransferase (DDOST)N-terminal signal polypeptide, for example, SEQ ID NO: 50 (MKMGVRLAARAWPLCGLLLAALGGVCA). In an aspect, the signal polypeptide is an N-terminal signal polypeptide expressing SEAP in CHO—S cells, for example, SEQ ID NO: 51 (MWWRLWWLLLLLLLLWLALAAAA). In an aspect, the signal polypeptide is an N-terminal signal polypeptide expressing rituximab HC in CHO K1 cells, for example, SEQ ID NO: 52 (MGWSLILLFLVAVATRVLS). In an aspect, the signal polypeptide is an N-terminal signal peptide expressing rituximab LC in CHO K1 cells, for example, SEQ ID NO: 53 (MDFQVQIISFLLISASVIMSRG). In an aspect, the signal polypeptide is an N-terminal signal polypeptide expressing avastin, humira, rituxan, and remicade HC in CHO K1 cells, for example, SEQ ID NO: 54 (MEFGLSWVFLVALFRGVQC). In an aspect, the signal polypeptide is a serum albumin preproprotein N-terminal signal polypeptide expressing model antibody HC and LC and a model fusion protein in CHO K1 cells, and Gaussia luciferase in CHO DG44 and CHO AA8 cells, for example, SEQ ID NO: 55 (MKWVTFISLLFLFSSAYS). In an aspect, the signal polypeptide is an N-terminal signal polypeptide expressing anti-HER2 antibody in CHO DG44 and E. coli W3110 cells, for example, SEQ ID NO: 56 (MKLPVRLLVLMFWIPAASA). In an aspect, the signal polypeptide is a human trypsinogen-2 N-terminal signal polypeptide expressing Gaussia luciferase in CHO cells, for example, SEQ ID NO: 57 (MNLLLILTFVAAAVA). In an aspect, the signal polypeptide is an N-terminal signal polypeptide derived from CHO comprising a modified Ig kappa chain V-III region MOPC63-like precursor with the last 4 amino acids taken from azurocidin preproproteinm, where the signal polypeptide expresses GFP and a model scFv-Fc in CHO K1 and CHO DG44 cells, for example, SEQ ID NO: 58 (MGSAALLLWVLLLWVPSSRA). In an aspect, the signal polypeptide is an N-terminal azurocidin preproprotein signal polypeptide expressing two model antibodies HCs and LCs and a model fusion protein, GFP and a model scFv-Fc in CHO K1 and CHO DG44 cells, for example, SEQ ID NO: 59 (MTRLTVLALLAGLLASSRA). In an aspect, the signal polypeptide is an N-terminal signal polypeptide expressing SEAP, IFNá2, IL-25, sclerostin, mimecan, and prostaglandin-H2 d-isomerase in HEK293 and CHO—S cells, for example, SEQ ID NO: 60 (MWWRLWWLLLLLLLLWPMVWAAA). In an aspect, the signal polypeptide is an N-terminal signal polypeptide expressing an anti-HER2 antibody and an anti-HER2 Fab in CHO DG44 and E. coli W3110 cells, for example, SEQ ID NO: 61 (MKLPVRLLVLMFWIPASSS). In an aspect, the signal polypeptide is an N-terminal signal polypeptide expressing avastin, rituxan, remicade, herceptin, and humira light and HCs in CHO K1 cells, for example, SEQ ID NO: 62 (MDMRVPAQLLGLLLLWLSGARC). In an aspect, the signal polypeptide is an N-terminal signal polypeptide expressing avastin, rituxan, remicade, herceptin and humira light and HCs in CHO K1 cells, for example, SEQ ID NO: 63 (MKYLLPTAAAGLLLLAAQPAMA). In an aspect, the signal polypeptide is an N-terminal native G. princeps signal peptide expressing Gaussia luciferase in CHO cells including CHO K1 and CHO AA8 cells, for example, SEQ ID NO: 64 (MGVKVLFALICIAVAEA). In an aspect, the signal polypeptide is an N-terminal CD33 signal polypeptide expressing SEAP in HEK293 cells, for example, SEQ ID NO: 65 (MPLLLLLPLLWAGALA). In an aspect, the signal polypeptide is a signal polypeptide of SEQ ID NO: 66 (MRARALLAVLLLLLLVGIAAAA). In an aspect, the signal polypeptide is a signal polypeptide of SEQ ID NO: 67 (MATATLLAVLLLLLLVGSAGGA). In an aspect, the signal polypeptide is a signal polypeptide of SEQ ID NO: 68 (MRARALLVVLVLVVLLGVASSA). In an aspect, the signal polypeptide is a signal polypeptide of SEQ ID NO: 69 (MPGPGAALLLLLLVLLGLGSAA). In an aspect, the signal polypeptide is a signal polypeptide of SEQ ID NO: 70 (MTTTTVLLLLVLVVLAGLTSGA).

In an aspect, B is a polypeptide that binds to polyA binding protein (PAPB). In an aspect, B is a cell penetrating polypeptide. In an aspect, B is selected from the group consisting of Paip1, Paip2 (QFGDFDPSVEEEEDL; SEQ ID NO: 71), and eRF3. In an aspect, B is Paip1. In an aspect, B is Paip2. In an aspect, B is eRF3.

b. mRNA Molecules

In an aspect, B is an oligonucleotide having a 3′-end and a 5′-end. In an aspect, B comprises a poly-adenosine monophosphate region or a poly-thymidine monophosphate region. In an aspect, B comprises a poly-adenosine monophosphate region. In an aspect, B comprises a poly-thymidine monophosphate region. In an aspect, the 5′-end of B is ligated to an mRNA molecule. In an aspect, B is an mRNA molecule.

In an aspect, B includes an mCherry open reading frame sequence:

(SEQ ID NO: 72)
AUGGUGAGCAAGGGCGAGGAGGACAACAUGGCCAUCAUCAAGGAGUUCAUGCGG
UUCAAGGUGCACAUGGAGGGCAGCGUGAACGGCCACGAGUUCGAGAUCGAGGGC
GAGGGCGAGGGCCGGCCCUACGAGGGCACCCAGACCGCCAAGCUGAAGGUGACCA
AGGGCGGCCCCCUGCCCUUCGCCUGGGACAUCCUGAGCCCCCAGUUCAUGUACGG
CAGCAAGGCCUACGUGAAGCACCCCGCCGACAUCCCCGACUACCUGAAGCUGAGC
UUCCCCGAGGGCUUCAAGUGGGAGCGGGUGAUGAACUUCGAGGACGGCGGCGUG
GUGACCGUGACCCAGGACAGCAGCCUGCAGGACGGCGAGUUCAUCUACAAGGUGA
AGCUGCGGGGCACCAACUUCCCCAGCGACGGCCCCGUGAUGCAGAAGAAGACCAU
GGGCUGGGAGGCCAGCAGCGAGCGGAUGUACCCCGAGGACGGCGCCCUGAAGGGC
GAGAUCAAGCAGCGGCUGAAGCUGAAGGACGGCGGCCACUACGACGCCGAGGUGA
AGACCACCUACAAGGCCAAGAAGCCCGUGCAGCUGCCCGGCGCCUACAACGUGAA
CAUCAAGCUGGACAUCACCAGCCACAACGAGGACUACACCAUCGUGGAGCAGUAC
GAGCGGGCCGAGGGCCGGCACAGCACCGGCGGCAUGGACGAGCUGUACAAGAGCG
GCAACUGA, a 5′-UTR region, a 3′-UTR region, and a PolyA tail.

In an aspect, B includes an eGFP open reading frame sequence:

(SEQ ID NO: 73)
AUGGUGAGCAAGGGCGAGGAGCUGUUCACCGGGGUGGUGCCCAUCCUGGUCGAG
CUGGACGGCGACGUAAACGGCCACAAGUUCAGCGUGUCCGGCGAGGGCGAGGGCG
AUGCCACCUACGGCAAGCUGACCCUGAAGUUCAUCUGCACCACCGGCAAGCUGCC
CGUGCCCUGGCCCACCCUCGUGACCACCCUGACCUACGGCGUGCAGUGCUUCAGC
CGCUACCCCGACCACAUGAAGCAGCACGACUUCUUCAAGUCCGCCAUGCCCGAAG
GCUACGUCCAGGAGCGCACCAUCUUCUUCAAGGACGACGGCAACUACAAGACCCG
CGCCGAGGUGAAGUUCGAGGGCGACACCCUGGUGAACCGCAUCGAGCUGAAGGGC
AUCGACUUCAAGGAGGACGGCAACAUCCUGGGGCACAAGCUGGAGUACAACUACA
ACAGCCACAACGUCUAUAUCAUGGCCGACAAGCAGAAGAACGGCAUCAAGGUGAA
CUUCAAGAUCCGCCACAACAUCGAGGACGGCAGCGUGCAGCUCGCCGACCACUAC
CAGCAGAACACCCCCAUCGGCGACGGCCCCGUGCUGCUGCCCGACAACCACUACC
UGAGCACCCAGUCCGCCCUGAGCAAAGACCCCAACGAGAAGCGCGAUCACAUGGU
CCUGCUGGAGUUCGUGACCGCCGCCGGGAUCACUCUCGGCAUGGACGAGCUGUAC
AAGUAA, a 5′-UTR region, a 3′-UTR region, and a PolyA tail.

In an aspect, B includes a FLuc open reading frame sequence:

(SEQ ID NO: 74)
AUGGAGGACGCCAAGAACAUCAAGAAGGGCCCCGCCCCCUUCUACCCCCUGGAGG
ACGGCACCGCCGGCGAGCAGCUGCACAAGGCCAUGAAGCGGUACGCCCUGGUGCC
CGGCACCAUCGCCUUCACCGACGCCCACAUCGAGGUGGACAUCACCUACGCCGAG
UACUUCGAGAUGAGCGUGCGGCUGGCCGAGGCCAUGAAGCGGUACGGCCUGAACA
CCAACCACCGGAUCGUGGUGUGCAGCGAGAACAGCCUGCAGUUCUUCAUGCCCGU
GCUGGGCGCCCUGUUCAUCGGCGUGGCCGUGGCCCCCGCCAACGACAUCUACAAC
GAGCGGGAGCUGCUGAACAGCAUGGGCAUCAGCCAGCCCACCGUGGUGUUCGUGA
GCAAGAAGGGCCUGCAGAAGAUCCUGAACGUGCAGAAGAAGCUGCCCAUCAUCCA
GAAGAUCAUCAUCAUGGACAGCAAGACCGACUACCAGGGCUUCCAGAGCAUGUAC
ACCUUCGUGACCAGCCACCUGCCCCCCGGCUUCAACGAGUACGACUUCGUGCCCG
AGAGCUUCGACCGGGACAAGACCAUCGCCCUGAUCAUGAACAGCAGCGGCAGCAC
CGGCCUGCCCAAGGGCGUGGCCCUGCCCCACCGGACCGCCUGCGUGCGGUUCAGC
CACGCCCGGGACCCCAUCUUCGGCAACCAGAUCAUCCCCGACACCGCCAUCCUGA
GCGUGGUGCCCUUCCACCACGGCUUCGGCAUGUUCACCACCCUGGGCUACCUGAU
CUGCGGCUUCCGGGUGGUGCUGAUGUACCGGUUCGAGGAGGAGCUGUUCCUGCG
GAGCCUGCAGGACUACAAGAUCCAGAGCGCCCUGCUGGUGCCCACCCUGUUCAGC
UUCUUCGCCAAGAGCACCCUGAUCGACAAGUACGACCUGAGCAACCUGCACGAGA
UCGCCAGCGGCGGCGCCCCCCUGAGCAAGGAGGUGGGCGAGGCCGUGGCCAAGCG
GUUCCACCUGCCCGGCAUCCGGCAGGGCUACGGCCUGACCGAGACCACCAGCGCC
AUCCUGAUCACCCCCGAGGGCGACGACAAGCCCGGCGCCGUGGGCAAGGUGGUGC
CCUUCUUCGAGGCCAAGGUGGUGGACCUGGACACCGGCAAGACCCUGGGCGUGAA
CCAGCGGGGCGAGCUGUGCGUGCGGGGCCCCAUGAUCAUGAGCGGCUACGUGAAC
AACCCCGAGGCCACCAACGCCCUGAUCGACAAGGACGGCUGGCUGCACAGCGGCG
ACAUCGCCUACUGGGACGAGGACGAGCACUUCUUCAUCGUGGACCGGCUGAAGAG
CCUGAUCAAGUACAAGGGCUACCAGGUGGCCCCCGCCGAGCUGGAGAGCAUCCUG
CUGCAGCACCCCAACAUCUUCGACGCCGGCGUGGCCGGCCUGCCCGACGACGACG
CCGGCGAGCUGCCCGCCGCCGUGGUGGUGCUGGAGCACGGCAAGACCAUGACCGA
GAAGGAGAUCGUGGACUACGUGGCCAGCCAGGUGACCACCGCCAAGAAGCUGCGG
GGCGGCGUGGUGUUCGUGGACGAGGUGCCCAAGGGCCUGACCGGCAAGCUGGACG
CCCGGAAGAUCCGGGAGAUCCUGAUCAAGGCCAAGAAGGGCGGCAAGAUCGCCGU
GUGA, a 5′-UTR region, a 3′-UTR region, and a PolyA tail.

In an aspect, B encodes a therapeutic polypeptide. In an aspect, B encodes a therapeutic polypeptide having anti-cancer activity. In an aspect, B encodes a therapeutic polypeptide having anti-obesity activity. In an aspect, B encodes an amino acid sequence selected from the group consisting of SEQ ID NOs: 1 to 71.

### 3. Cargo (A)

In an aspect, A is an oligonucleotide having a 3′-end and a 5′-end. In an aspect, A comprises a poly-adenosine monophosphate region or a poly-thymidine monophosphate region. In an aspect, A comprises a poly-adenosine monophosphate region. In an aspect, A comprises a poly-thymidine monophosphate region. In an aspect, the 5′-end of A is ligated to an mRNA molecule. In an aspect, A is an mRNA molecule.

In an aspect, A is mCherry mRNA having the nucleotide sequence of SEQ ID NO: 72.

In an aspect, A is mCherry mRNA (SEQ ID NO: 72) having a 5′-UTR region, a 3′-UTR region, and PolyA tail attached.

In an aspect, A is eGFP mRNA having the nucleotide sequence of SEQ ID NO: 73.

In an aspect, A is eGFP mRNA (SEQ ID NO: 73) having a 5′-UTR region, a 3′-UTR region, and PolyA tail attached.

In an aspect, A is FLuc mRNA having the nucleotide sequence of SEQ ID NO: 74.

In an aspect, A is FLuc mRNA (SEQ ID NO: 74) having a 5′-UTR region, a 3′-UTR region, and PolyA tail attached.

In an aspect, A encodes a therapeutic polypeptide. In an aspect, A encodes a therapeutic polypeptide having anti-cancer activity. In an aspect, A encodes a therapeutic polypeptide having anti-obesity activity. In an aspect, A encodes an amino acid sequence selected from the group consisting of SEQ ID NOs: 1 to 71.

In an aspect, A encodes an antibody. In aspect, A encodes the light chain of an antibody. In aspect, A encodes the heavy chain of an antibody. In an aspect, A encodes an antibody selected from the group consisting of adalimumab, dupilumab, trastuzumab, and pembrolizumab. In an aspect, A comprises a sequence selected from the group consisting of SEQ ID NOs: 108, 110, 112, 114, 116, and 118. In an aspect, A encodes an amino acid sequence selected from the group consisting of SEQ ID Nos: 109, 111, 113, 115, 117, and 119.

In an aspect, A encodes a peptide. In an aspect, A encodes a peptide selected from the group consisting of exenatide, GLP-1, gastric inhibitory peptide, and teriparatide. In an aspect, A comprises a sequence selected from the group consisting of SEQ ID NOs: 120, 122, 124, and 126. In an aspect, A encodes an amino acid sequence selected from the group consisting of SEQ ID Nos: 121, 123, 125, and 127.

### 4. Second Linkage

In an aspect, B is an oligonucleotide having a 3′-end and a 5′-end. In an aspect, B comprises a poly-adenosine monophosphate region or a poly-thymidine monophosphate region. In an aspect, B comprises a poly-adenosine monophosphate region. In an aspect, B comprises a poly-thymidine monophosphate region. In an aspect, B is an mRNA molecule. In an aspect, B is a DNA molecule. In an aspect, Z is covalently attached to the 3′-end of B through a second linkage selected from the group consisting of a phosphate linkage, a phosphorothioate linkage, a phosphoramidate linkage, an amine linkage, an amide linkage, a triazole linkage, an ether linkage, and a thioether linkage.

In an aspect, B is an oligonucleotide having a 3′-end and a 5′-end, and Z is covalently attached to the 3′-end of B through a second linkage that is a phosphate linkage. An example of the covalent attachment of a nucleotide located at the 3′-end of B through a phosphate linkage to Z is shown below in formula (K)

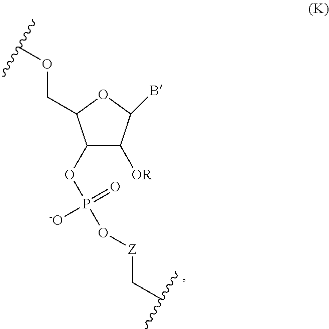

where B′ is a natural or non-natural nucleobase, and R is H, an alcohol protecting group, or a C₁-C₆alkyl, where any one or more —CH₂— groups of the C₁-C₆ alkyl is each optionally replaced independently with —CHF—, —CF₂—, or a heteroatomic moiety selected from the group consisting of —O— and —S—, and where any two heteroatomic moieties are separated from one another by at least two carbon atoms. In an aspect, a nucleotide located at the 3′-end of B comprises the phosphate linkage which covalently links B to Z. In an aspect, B comprises the phosphate linkage which covalently links A to Z. In an aspect, the phosphate linkage between Z and B comprises a phosphate attached to the 3′-oxygen atom of a nucleotide located at the 3′-end of B. In an aspect, the oligonucleotide bioconjugate of formula (I) comprises formula (K). In an aspect, the oligonucleotide bioconjugate of formula (II) comprises formula (K). In an aspect, the oligonucleotide bioconjugate of formula (III) comprises formula (K). In an aspect, the oligonucleotide bioconjugate of formula (IV) comprises formula (K). In an aspect, the oligonucleotide bioconjugate of formula (V) comprises formula (K).

In an aspect, B is an oligonucleotide having a 3′-end and a 5′-end, and Z is covalently attached to the 3′-end of B through a second linkage that is a phosphorothioate linkage. An example of the covalent attachment of a nucleotide located at the 3′-end of B through a phosphorothioate linkage to Z is shown below in formula (L)

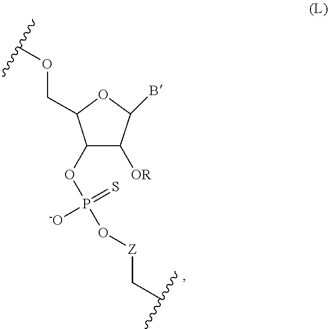

where B′ is a natural or non-natural nucleobase, and R is H, an alcohol protecting group, or a C₁-C₆alkyl, where any one or more —CH₂— groups of the C₁-C₆ alkyl is each optionally replaced independently with —CHF—, —CF₂—, or a heteroatomic moiety selected from the group consisting of —O— and —S—, and where any two heteroatomic moieties are separated from one another by at least two carbon atoms. In an aspect, a nucleotide located at the 3′-end of B comprises the phosphorothioate linkage which covalently links B to Z. In an aspect, B comprises the phosphorothioate linkage which covalently links B to Z. In an aspect, the phosphorothioate linkage between Z and B comprises a phosphorothioate attached to the 3′-oxygen atom of a nucleotide located at the 3′-end of B. In an aspect, the oligonucleotide bioconjugate of formula (I) comprises formula (L). In an aspect, the oligonucleotide bioconjugate of formula (II) comprises formula (L). In an aspect, the oligonucleotide bioconjugate of formula (III) comprises formula (L). In an aspect, the oligonucleotide bioconjugate of formula (IV) comprises formula (L). In an aspect, the oligonucleotide bioconjugate of formula (V) comprises formula (L).

In an aspect, B is an oligonucleotide having a 3′-end and a 5′-end, and Z is covalently attached to the 3′-end of B through a second linkage that is a phosphoramidate linkage. An example of the covalent attachment of a nucleotide located at the 3′-end of B through a phosphoramidate linkage to Z is shown below in formula (M)

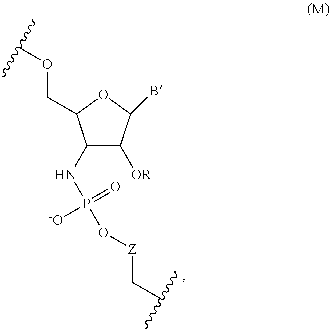

where B′ is a natural or non-natural nucleobase, and R is H, an alcohol protecting group, or a C₁-C₆alkyl, where any one or more —CH₂— groups of the C₁-C₆ alkyl is each optionally replaced independently with —CHF—, —CF₂—, or a heteroatomic moiety selected from the group consisting of —O— and —S—, and where any two heteroatomic moieties are separated from one another by at least two carbon atoms. In an aspect, a nucleotide located at the 3′-end of B comprises the phosphoramidate linkage which covalently links B to Z. In an aspect, B comprises the phosphoramidate linkage which covalently links B to Z. In an aspect, the phosphoramidate linkage between Z and B comprises a phosphoramidate attached to the 3′-oxygen atom of a nucleotide located at the 3′-end of B. In an aspect, the oligonucleotide bioconjugate of formula (I) comprises formula (M). In an aspect, the oligonucleotide bioconjugate of formula (II) comprises formula (M). In an aspect, the oligonucleotide bioconjugate of formula (III) comprises formula (M). In an aspect, the oligonucleotide bioconjugate of formula (IV) comprises formula (M). In an aspect, the oligonucleotide bioconjugate of formula (V) comprises formula (M).

In an aspect, B is an oligonucleotide having a 3′-end and a 5′-end, and Z is covalently attached to the 3′-end of B through a second linkage that is an amine linkage. An example of the covalent attachment of a nucleotide located at the 3′-end of B through an amine linkage to Z is shown below in formula (N)

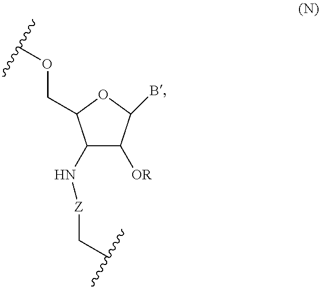

where B′ is a natural or non-natural nucleobase, and R is H, an alcohol protecting group, or a C₁-C₆alkyl, where any one or more —CH₂— groups of the C₁-C₆ alkyl is each optionally replaced independently with —CHF—, —CF₂—, or a heteroatomic moiety selected from the group consisting of —O— and —S—, and where any two heteroatomic moieties are separated from one another by at least two carbon atoms. In an aspect, a nucleotide located at the 3′-end of B comprises the amine linkage which covalently links B to Z. In an aspect, A comprises the amine linkage which covalently links B to Z. In an aspect, the amine linkage between B and Z comprises an amine attached to the 3′-position of a nucleotide located at the 3′-end of B. In an aspect, the oligonucleotide bioconjugate of formula (I) comprises formula (N). In an aspect, the oligonucleotide bioconjugate of formula (II) comprises formula (N). In an aspect, the oligonucleotide bioconjugate of formula (III) comprises formula (N). In an aspect, the oligonucleotide bioconjugate of formula (IV) comprises formula (N). In an aspect, the oligonucleotide bioconjugate of formula (V) comprises formula (N).

In an aspect, B is an oligonucleotide having a 3′-end and a 5′-end, and Z is covalently attached to the 3′-end of B through a second linkage that is an amide linkage. An example of the covalent attachment of a nucleotide located at the 3′-end of B through an amide linkage to Z is shown below in formula (O)

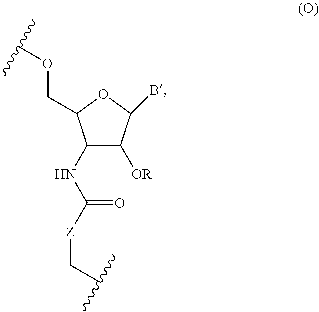

where B′ is a natural or non-natural nucleobase, and R is H or a C₁-C₆ alkyl, where any one or more —CH₂— groups of the C₁-C₆ alkyl is each optionally replaced independently with —CHF—, —CF₂—, or a heteroatomic moiety selected from the group consisting of —O— and —S—, and where any two heteroatomic moieties are separated from one another by at least two carbon atoms. In an aspect, a nucleotide located at the 3′-end of B comprises the amide linkage which covalently links B to Z. In an aspect, the final nucleotide located at the 3′-end of B comprises the NH of the amide linkage which covalently links B to Z. In an aspect, A comprises the amide group which covalently links B to Z. In an aspect, A comprises the NH of the amide group which covalently links B to Z. In an aspect, the amide linkage between Z and B comprises an amide attached to the 3′-position of a nucleotide located at the 3′-end of B. In an aspect, the oligonucleotide bioconjugate of formula (I) comprises formula (O). In an aspect, the oligonucleotide bioconjugate of formula (II) comprises formula (O). In an aspect, the oligonucleotide bioconjugate of formula (III) comprises formula (O). In an aspect, the oligonucleotide bioconjugate of formula (IV) comprises formula (O). In an aspect, the oligonucleotide bioconjugate of formula (V) comprises formula (O).

In an aspect, B is an oligonucleotide having a 3′-end and a 5′-end, and Z is covalently attached to the 3′-end of B through a second linkage that is a triazole linkage. Examples of the covalent attachment of a nucleotide located at the 3′-end of B through a triazole linkage to Z are shown below in formula (P) and formula (Q)

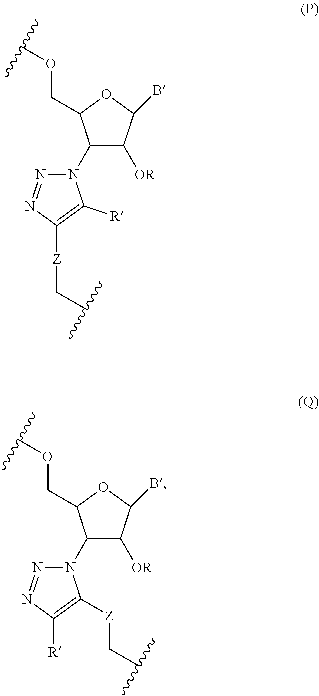

where B′ is a natural or non-natural nucleobase, R is H, an alcohol protecting group, or a C₁-C₆ alkyl, where any one or more —CH₂— groups of the C₁-C₆ alkyl is each optionally replaced independently with —CHF—, —CF₂—, or a heteroatomic moiety selected from the group consisting of —O— and —S—, and where any two heteroatomic moieties are separated from one another by at least two carbon atoms, and R′ is selected from the group consisting of H, C₁-C₁₂ alkyl, C₁-C₁₂ alkenyl, C₁-C₁₂ alkynyl, and C₁-C₁₂ cycloalkyl. In an aspect, a nucleotide located at the 3′-end of B comprises the triazole linkage which covalently links B to Z. In an aspect, B comprises the triazole linkage which covalently links B to Z. In an aspect, the triazole linkage between Z and B comprises a triazole attached to the 3′-position of a nucleotide located at the 3′-end of B. In an aspect, the oligonucleotide bioconjugate of formula (I) comprises formula (P) or formula (Q). In an aspect, the oligonucleotide bioconjugate of formula (II) comprises formula (P) or formula (Q). In an aspect, the oligonucleotide bioconjugate of formula (III) comprises formula (P) or formula (Q). In an aspect, the oligonucleotide bioconjugate of formula (IV) comprises formula (P) or formula (Q). In an aspect, the oligonucleotide bioconjugate of formula (V) comprises formula (P) or formula (Q).

In an aspect, B is an oligonucleotide having a 3′-end and a 5′-end, and Z is covalently attached to the 3′-end of B through a second linkage that is an ether linkage. An example of the covalent attachment of a nucleotide located at the 3′-end of B through an ether linkage to Z is shown below in formula (R)

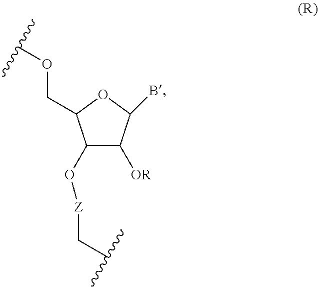

where B′ is a natural or non-natural nucleobase, and R is H, an alcohol protecting group, or a C₁-C₆alkyl, where any one or more —CH₂— groups of the C₁-C₆ alkyl is each optionally replaced independently with —CHF—, —CF₂—, or a heteroatomic moiety selected from the group consisting of —O— and —S—, and where any two heteroatomic moieties are separated from one another by at least two carbon atoms. In an aspect, a nucleotide located at the 3′-end of B comprises the ether linkage which covalently links B to Z. In an aspect, B comprises the ether linkage which covalently links B to Z. In an aspect, the ether linkage between B and Z comprises a bond to the 3′-oxygen atom of a nucleotide located at the 3′-end of B. In an aspect, the oligonucleotide bioconjugate of formula (I) comprises formula (R). In an aspect, the oligonucleotide bioconjugate of formula (II) comprises formula (R). In an aspect, the oligonucleotide bioconjugate of formula (III) comprises formula (R). In an aspect, the oligonucleotide bioconjugate of formula (IV) comprises formula (R). In an aspect, the oligonucleotide bioconjugate of formula (V) comprises formula (R).

In an aspect, B is an oligonucleotide having a 3′-end and a 5′-end, and Z is covalently attached to the 3′-end of B through a second linkage that is a thioether linkage. An example of the covalent attachment of a nucleotide located at the 3′-end of B through a thioether linkage to Z is shown below in formula (S)

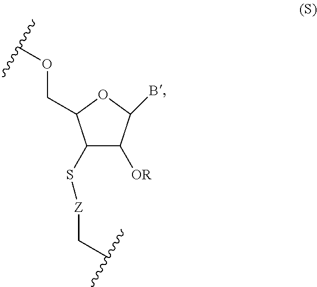

where B′ is a natural or non-natural nucleobase, and R is H, an alcohol protecting group, or a C₁-C₆alkyl, where any one or more —CH₂— groups of the C₁-C₆ alkyl is each optionally replaced independently with —CHF—, —CF₂—, or a heteroatomic moiety selected from the group consisting of —O— and —S—, and where any two heteroatomic moieties are separated from one another by at least two carbon atoms. In an aspect, a nucleotide located at the 3′-end of B comprises the thioether linkage which covalently links B to Z. In an aspect, B comprises the thioether linkage which covalently links B to Z. In an aspect, the thioether linkage between B and Z comprises a thioether attached to the 3′-position of a nucleotide located at the 3′-end of B. In an aspect, the oligonucleotide bioconjugate of formula (I) comprises formula (S). In an aspect, the oligonucleotide bioconjugate of formula (II) comprises formula (S). In an aspect, the oligonucleotide bioconjugate of formula (III) comprises formula (S). In an aspect, the oligonucleotide bioconjugate of formula (IV) comprises formula (S). In an aspect, the oligonucleotide bioconjugate of formula (V) comprises formula (S).

### 5. Y

In an aspect, Y is —CH₂—. In an aspect, Y is —CH₂CH₂—. In an aspect, Y is —CH₂CH₂CH₂—. In an aspect, Y is *—(CH₂)₂O(CH₂)₂—, wherein * indicates the attachment point to A. In an aspect, Y is *—(CH₂)₂O(CH₂)₂O(CH₂)₂—, wherein * indicates the attachment point to A. In an aspect, Y is *—(CH₂)₂S(CH₂)₂—, wherein * indicates the attachment point to A. In an aspect, Y is *—(CH₂)₂S(CH₂)₂S(CH₂)₂—, wherein * indicates the attachment point to A. In an aspect, Y is *—(CH₂)₂N(C₁-C₆ alkyl)(CH₂)₂—, wherein * indicates the attachment point to A. In an aspect, Y is *—(CH₂)₂N(C₁-C₆ alkyl)(CH₂)₂N(C₁-C₆ alkyl)(CH₂)₂—, wherein * indicates the attachment point to A. In an aspect, Y is —CH₂CH(OCH₃)CH₂—. In an aspect, Y is —CH₂CH(N(C₁-C₆ alkyl))CH₂—. In an aspect, Y comprises a polyamide moiety. In an aspect, Y comprises an acyl group. In an aspect, Y comprises an amide. In an aspect, Y comprises an ether. In an aspect, Y comprises an polyether. In an aspect, Y comprises a thioether. In an aspect, Y comprises an amine. In an aspect, Y comprises an polyamine. In an aspect, Y comprises a polyethylene glycol (PEG) moiety.

### 6. Z

In an aspect, Z is —CH₂—. In an aspect, Z is —CH₂CH₂—. In an aspect, Z is —CH₂CH₂CH₂—. In an aspect, Z is *—(CH₂)₂O(CH₂)₂—, wherein * indicates the attachment point to B. In an aspect, Z is *—(CH₂)₂O(CH₂)₂O(CH₂)₂—, wherein * indicates the attachment point to B. In an aspect, Z is *—(CH₂)₂S(CH₂)₂—, wherein * indicates the attachment point to B. In an aspect, Z is *—(CH₂)₂S(CH₂)₂S(CH₂)₂—, wherein * indicates the attachment point to B. In an aspect, Z is *—(CH₂)₂N(C₁-C₆ alkyl)(CH₂)₂—, wherein * indicates the attachment point to B. In an aspect, Z is *—(CH₂)₂N(C₁-C₆ alkyl)(CH₂)₂N(C₁-C₆ alkyl)(CH₂)₂—, wherein * indicates the attachment point to B. In an aspect, Z is —CH₂CH(OCH₃)CH₂—. In an aspect, Z is —CH₂CH(N(C₁-C₆ alkyl))CH₂—. In an aspect, Z comprises a polyamide moiety. In an aspect, Z comprises an acyl group. In an aspect, Z comprises an amide. In an aspect, Z comprises an ether. In an aspect, Z comprises an polyether. In an aspect, Z comprises a thioether. In an aspect, Z comprises an amine. In an aspect, Z comprises an polyamine. In an aspect, Z comprises a polyethylene glycol (PEG) moiety.

### 7. L

In an aspect, L is a C₂-C₅₀ alkyl. In an aspect, L is a C₂-C₅₀ alkenyl. In an aspect, L is a C₂-C₅₀ alkynyl. In an aspect, L is a C₃-C₈ cycloalkyl. In an aspect, L comprises a polyethylene glycol (PEG) moiety. In an aspect, L is a PEG₃ moiety. In an aspect, L is a PEG₁₉ moiety. In an aspect, L comprises a polyamide moiety. In an aspect, L is a polypeptide. In an aspect, L comprises an acyl group. In an aspect, L comprises an amide. In an aspect, L comprises an ether. In an aspect, L comprises an polyether. In an aspect, L comprises a thioether. In an aspect, L comprises an amine. In an aspect, L is an aryl group. In an aspect, L comprises an aryl group. In an aspect, L comprises a disubstituted aryl group. In an aspect, L comprises a trisubstituted aryl group. In an aspect, L comprises a tetrasubstituted aryl group. In an aspect, L is a heteroaryl group. In an aspect, L comprises a heteroaryl group. In an aspect, L is a C₄-C₅₀ alkyl-cycloalkyl. In an aspect, L is a C₇-C₅₀ alkyl-aryl. In an aspect, L is a C₆-C₅₀ alkyl-heteroaryl.

### 8. Conjugated Carbohydrates

Oligonucleotide bioconjugates of formula (I) or formula (II), or oligonucleotide precursors thereof, may comprise one or more attached carbohydrates. Without being limited by theory, the attachment of carbohydrates to an oligonucleotide bioconjugate, or a precursor thereof, may enhance tissue targeting or intracellular targeting. In an aspect, the one or more carbohydrates are attached to an mRNA molecule. In an aspect, the one or more carbohydrates are attached to the 3′-end of an mRNA molecule. In an aspect, the one or more carbohydrates are attached near the 3′-end of an mRNA molecule. In an aspect, the one or more carbohydrates are attached to A. In an aspect, the one or more carbohydrates are attached to the 3′-end of A. In an aspect, the one or more carbohydrates are attached near the 3′-end of A. In an aspect, the one or more carbohydrates are attached to B. In an aspect, the one or more carbohydrates are attached to the 3′-end of B. In an aspect, the one or more carbohydrates are attached near the 3′-end of B. In an aspect, the one or more carbohydrates are attached prior to bioconjugation. In an aspect, the one or more carbohydrates are attached during the bioconjugation process. In an aspect, the one or more carbohydrates are attached after bioconjugation. In an aspect, the one or more carbohydrates are covalently attached to an mRNA molecule, e.g., A or B. In an aspect, the one or more carbohydrates are covalently attached to the 3′-end of an mRNA molecule, e.g., A or B. In an aspect, the one or more carbohydrates are covalently attached near the 3′-end of an mRNA molecule, e.g., A or B.

In an aspect, each of the one or more carbohydrates is an O-linked carbohydrate. In an aspect, each of the one or more carbohydrates is an N-linked carbohydrate. In an aspect, the one or more carbohydrates are a combination of O-linked carbohydrates and N-linked carbohydrates. In an aspect, each of the one or more carbohydrates is a D-isomer. In an aspect, each of the one or more carbohydrates is an L-isomer. In an aspect, the one or more carbohydrates are a mixture of D- and L-isomers. In an aspect, one carbohydrate is attached to an oligonucleotide bioconjugate of formula (I) or formula (II), or an oligonucleotide precursor thereof. In an aspect, two carbohydrates are attached to an oligonucleotide bioconjugate of formula (I) or formula (II), or an oligonucleotide precursor thereof. In an aspect, three carbohydrates are attached to an oligonucleotide bioconjugate of formula (I) or formula (II), or an oligonucleotide precursor thereof. In an aspect, four carbohydrates are attached to an oligonucleotide bioconjugate of formula (I) or formula (II), or an oligonucleotide precursor thereof. In an aspect five carbohydrates are attached to an oligonucleotide bioconjugate of formula (I) or formula (II), or an oligonucleotide precursor thereof. In an aspect, six carbohydrates are attached to an oligonucleotide bioconjugate of formula (I) or formula (II), or an oligonucleotide precursor thereof. In an aspect, seven carbohydrates are attached to an oligonucleotide bioconjugate of formula (I) or formula (II), or an oligonucleotide precursor thereof. In an aspect, eight glycans carbohydrates are to an oligonucleotide bioconjugate of formula (I) or formula (II), or an oligonucleotide precursor thereof. In an aspect, nine carbohydrates are attached to an oligonucleotide bioconjugate of formula (I) or formula (II), or an oligonucleotide precursor thereof. In an aspect, ten carbohydrates are attached to an oligonucleotide bioconjugate of formula (I) or formula (II), or an oligonucleotide precursor thereof. In an aspect, eleven carbohydrates are attached to an oligonucleotide bioconjugate of formula (I) or formula (II), or an oligonucleotide precursor thereof. In an aspect, twelve carbohydrates are attached to an oligonucleotide bioconjugate of formula (I) or formula (II), or an oligonucleotide precursor thereof. In an aspect, one or two carbohydrates are attached to an oligonucleotide bioconjugate of formula (I) or formula (II), or an oligonucleotide precursor thereof. In an aspect, between one and three carbohydrates are attached to an oligonucleotide bioconjugate of formula (I) or formula (II), or an oligonucleotide precursor thereof. In an aspect, between one and six carbohydrates are attached to an oligonucleotide bioconjugate of formula (I) or formula (II), or an oligonucleotide precursor thereof. In an aspect, between three and six carbohydrates are attached to an oligonucleotide bioconjugate of formula (I) or formula (II), or an oligonucleotide precursor thereof. In an aspect, between one and 12 carbohydrates are attached to an oligonucleotide bioconjugate of formula (I) or formula (II), or an oligonucleotide precursor thereof. In an aspect, two or more carbohydrates are covalently attached to the oligonucleotide bioconjugate, or an oligonucleotide precursor thereof, in a linear arrangement. In an aspect, three or more carbohydrates are covalently attached to the oligonucleotide bioconjugate, or an oligonucleotide precursor thereof, in a branched arrangement. In an aspect, a primary hydroxyl group of a carbohydrate is covalently attached to the oligonucleotide bioconjugate, or an oligonucleotide precursor thereof. In an aspect, covalent attachment of the primary hydroxyl group of the carbohydrate to the oligonucleotide bioconjugate, or an oligonucleotide precursor thereof, is mediated by activation of the primary hydroxyl group via controlled periodate oxidation. In an aspect, a hydroxyl group at the C1 position of a carbohydrate is covalently attached to the oligonucleotide bioconjugate, or an oligonucleotide precursor thereof. In an aspect, covalent attachment of the hydroxyl group at the C1 position of the carbohydrate to the oligonucleotide bioconjugate, or an oligonucleotide precursor thereof, is mediated by oxidation. In an aspect, a carboxylate group of a carbohydrate is covalently attached to the oligonucleotide bioconjugate, or an oligonucleotide precursor thereof. In an aspect, covalent attachment of the carboxylate group of the carbohydrate to the oligonucleotide bioconjugate, or an oligonucleotide precursor thereof, is mediated by esterification. In an aspect, a vicinal diol of a carbohydrate is covalently attached to the oligonucleotide bioconjugate, or an oligonucleotide precursor thereof. In an aspect, the carbohydrate comprising a vicinal diol is a sialic acid. In an aspect, an orthogonal reactive group of a carbohydrate is condensed with the oligonucleotide bioconjugate, or an oligonucleotide precursor thereof. In an aspect, the orthogonal reactive group is an azide. In an aspect, the carbohydrate comprising an orthogonal reactive group is 2-azido-2-deoxy-D-glucose.

In an aspect, a carbohydrate of the present disclosure is a monosaccharide. In an aspect, a carbohydrate of the present disclosure comprises a monosaccharide. In an aspect, the monosaccharide is selected from the group consisting of glucose, galactose, mannose, allose, altrose, gulose, idose, talose, ribose, arabinose, xylose, and lyxose. In an aspect, the monosaccharide is glucose. In an aspect, the monosaccharide is galactose. In an aspect, the monosaccharide is mannose. In an aspect, the monosaccharide is allose. In an aspect, the monosaccharide is altrose. In an aspect, the monosaccharide is gulose. In an aspect, the monosaccharide is idose. In an aspect, the monosaccharide is talose. In an aspect, the monosaccharide is ribose. In an aspect, the monosaccharide is arabinose. In an aspect, the monosaccharide is xylose. In an aspect, the monosaccharide is lyxose. In an aspect, the monosaccharide is an N-acetyl sugar. In an aspect, the N-acetyl sugar is Glc-NAc or GalNAc. In an aspect, the N-acetyl sugar is Glc-NAc. In an aspect, the N-acetyl sugar is GalNAc. In an aspect, the monosaccharide is an alcohol sugar. In an aspect, the alcohol sugar is erythritol or mannitol. In an aspect, the alcohol sugar is erythritol. In an aspect, the alcohol sugar is mannitol. In an aspect, the monosaccharide is an acid sugar. In an aspect, the acid sugar is glucuronic acid or iduronic acid. In an aspect, the monosaccharide is a phosphorylated sugar. In aspect, the phosphorylated sugar is mannose-6-phosphate. In an aspect, the monosaccharide is a sulfated sugar. In aspect, the sulfated sugar is mannose-6-sulfate. In an aspect, the monosaccharide is a deoxy sugar. In an aspect, the deoxy sugar is rhamnose. In an aspect, the monosaccharide is a di-deoxy sugar. In an aspect, the di-deoxy sugar is olivose. In an aspect, the monosaccharide is mannose-derived sugar. In an aspect, the mannose-derived sugar is selected from the group consisting of mannose-6-phosphate, mannosamine, and Man-NAc. In an aspect, the mannose-derived sugar is mannose-6-phosphate. In an aspect, the mannose-derived sugar is mannosamine. In an aspect, the mannose-derived sugar is Man-NAc. In an aspect, the monosaccharide is an amino sugar. In an aspect, the amino sugar is galactosamine or glucosamine. In an aspect, the amino sugar is galactosamine. In an aspect, the amino sugar is glucosamine.

In an aspect, a carbohydrate of the present disclosure is a polysaccharide. In an aspect, a carbohydrate of the present disclosure comprises a polysaccharide. In an aspect, the polysaccharide is a linear polysaccharide. In an aspect, the polysaccharide is a branched polysaccharide. In an aspect the polysaccharide is a high mannose branched polysaccharide. In an aspect, the high mannose branched polysaccharide comprises an α(1,2) glycosidic linkage. In an aspect, the high mannose branched polysaccharide comprises an α(1,6) glycosidic linkage. In an aspect, the high mannose branched polysaccharide comprises an α(1,3) glycosidic linkage. In an aspect, the high mannose branched polysaccharide comprises a β(1,4) glycosidic linkage. In an aspect, the high mannose branched sugar is Man-3 GlcNAc2:

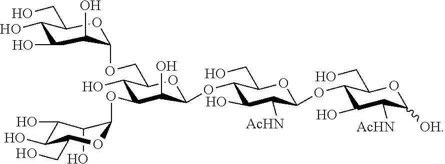

In an aspect, the high mannose branched polysaccharide is Man-6 GlcNAc2:

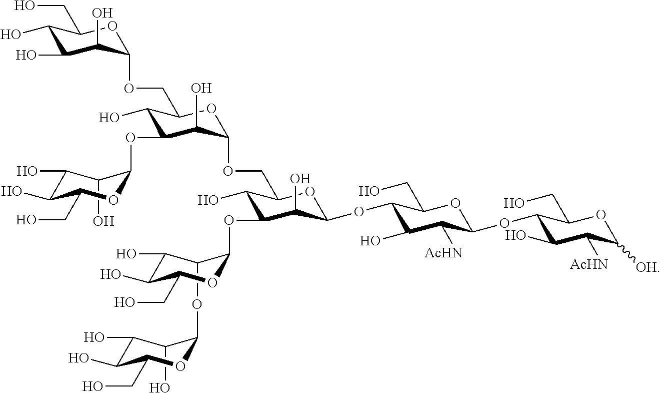

In an aspect, the high mannose branched polysaccharide is Man-9 GlcNAc2:

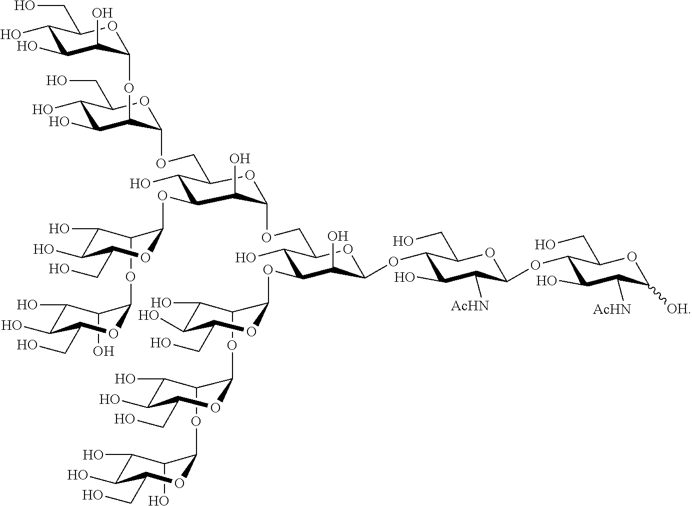

In an aspect, a carbohydrate of the present disclosure is a multi-antennary sugar. In an aspect, a carbohydrate of the present disclosure comprises a multi-antennary sugar. In an aspect, the multi-antennary sugar is a tri-antennary GalNAc sugar. In an aspect, the tri-antennary GalNAc sugar is:

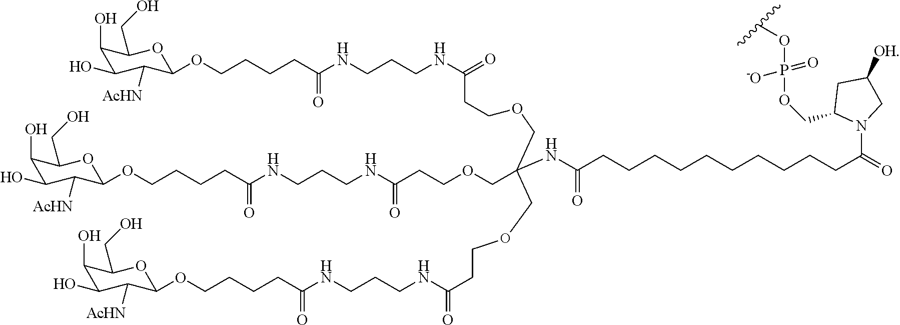

In an aspect, the multi-antennary sugar is a tri-antennary Man-6-P sugar. In an aspect, the tri-antennary Man-6-P sugar is:

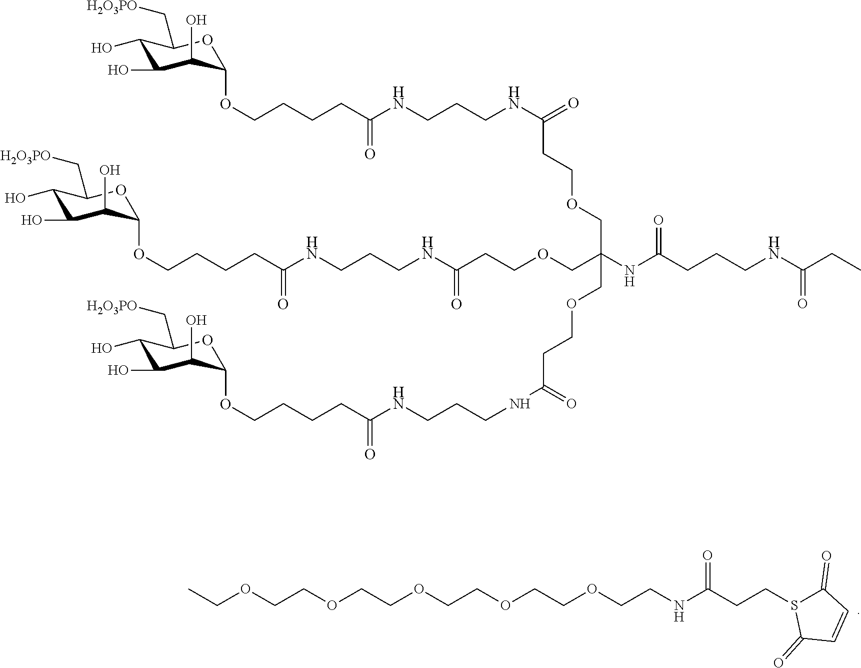

In an aspect, a carbohydrate of the present disclosure is a sialic acid. In an aspect, a carbohydrate of the present disclosure comprises a sialic acid. In an aspect, the sialic acid is N-acetylneuraminic acid:

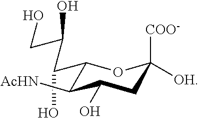

In an aspect, the sialic acid is Neu5Ac-α(2-3)-Gal-β(1-4)-GlcNAc-β-propylamine:

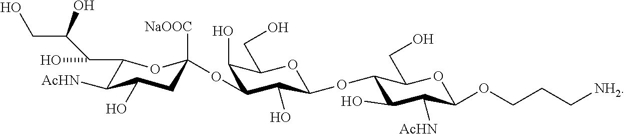

In an aspect, the covalent attachment is via an incorporated modified poly-A linker containing a sulfhydryl group. An example of a sulfide linkage to an oligonucleotide is shown below:

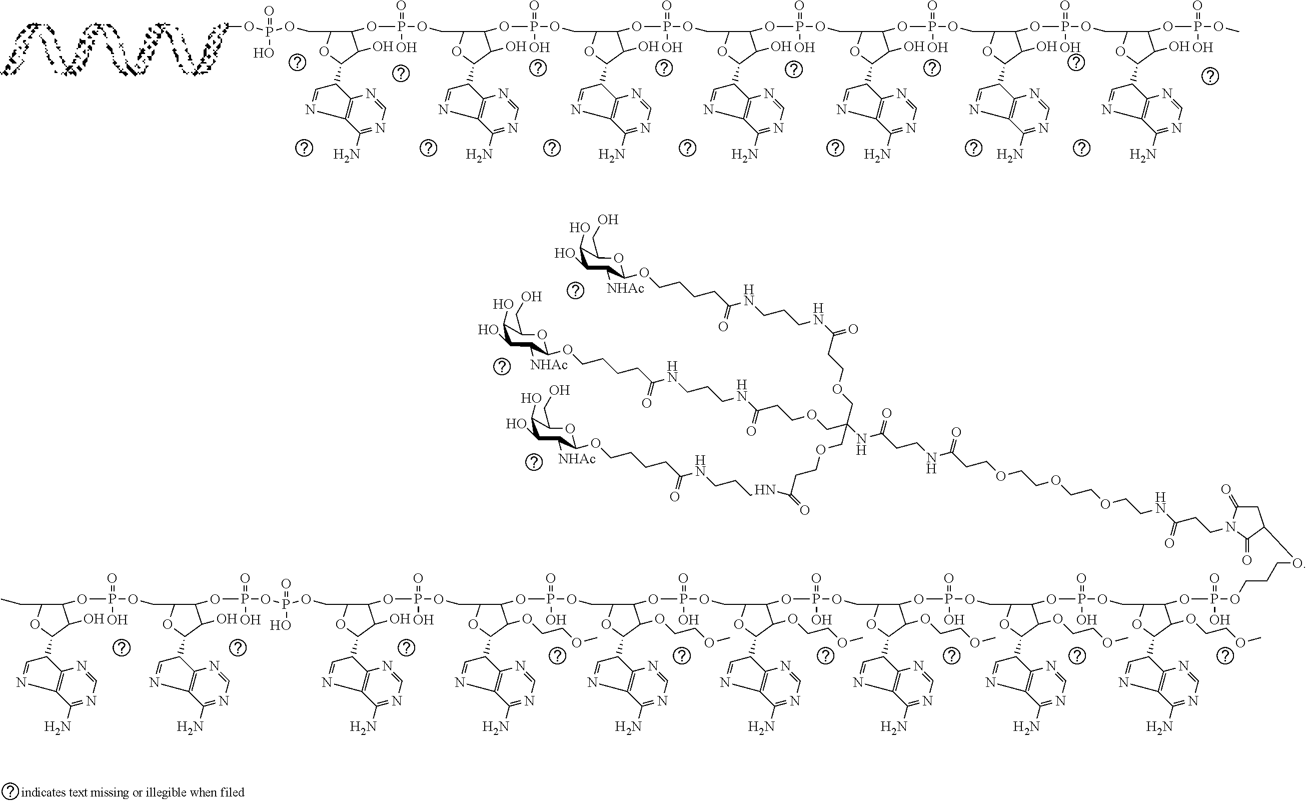

In an aspect, the covalent attachment is via a peptide containing internal reactive side-chains or terminal end (e.g., carboxyl, amine, sulfhydral, azide, or other groups). An example of a peptide linkage to an oligonucleotide-protein bioconjugate is shown below:

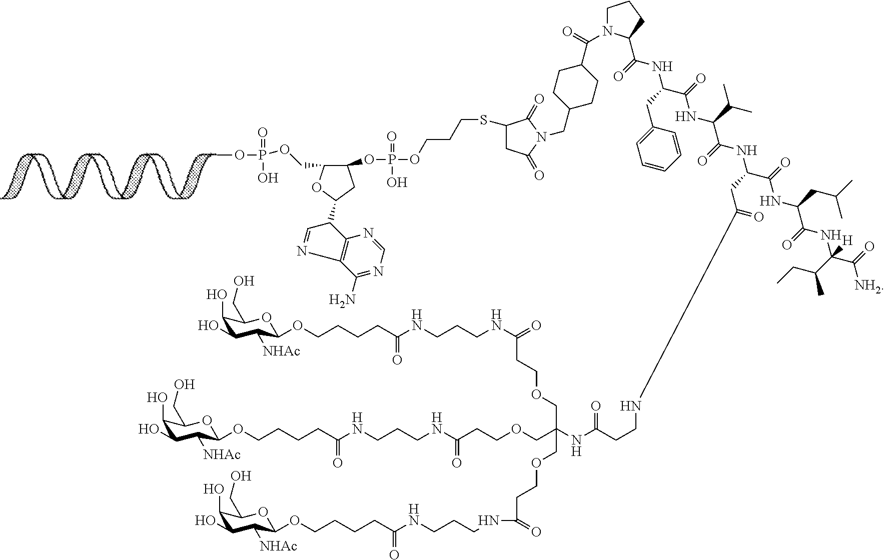

In an aspect, the covalent attachment is via an incorporated thiol-derived linker at the 3′-end of an oligonucleotide. An example of a sulfide linkage to the 3′-end of an oligonucleotide bioconjugate is shown below:

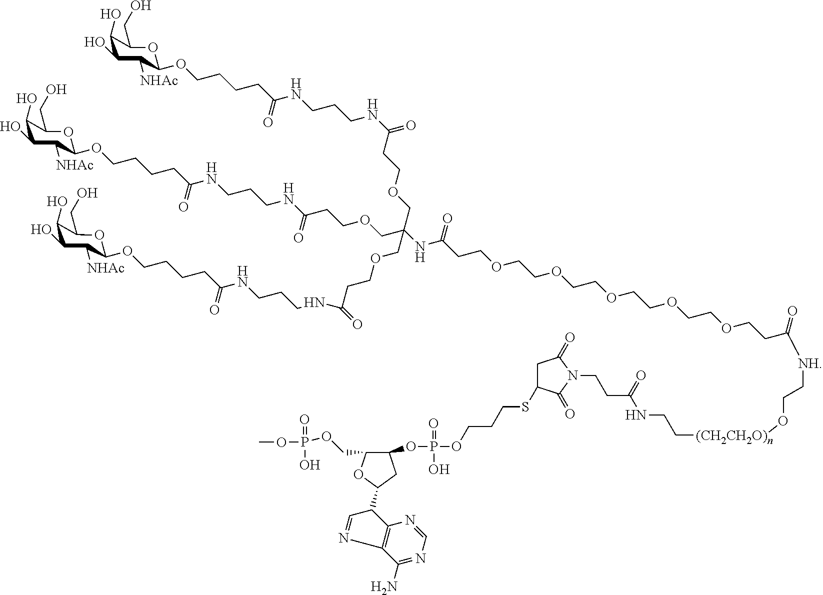

The oligonucleotide bioconjugates, mRNA bioconjugates, small molecules, and compounds of the present disclosure may be stereochemically enriched. Conventional techniques for the preparation and isolation of individual enantiomers include chiral synthesis from a suitable optically pure precursor, and resolution of the racemate (or the racemate of a salt or a derivative, using, for example, chiral high pressure liquid chromatography (HPLC). Alternatively, the racemate may be reacted or coordinated with a suitable optically active compound and then separated by chromatography or fractional crystallization. The stereochemical purities of an oligonucleotide bioconjugate, mRNA bioconjugate, small molecule, or compound of the present disclosure may be determined by standard analytical methods. Analytical methods to determine the diastereomeric excess of an oligonucleotide bioconjugate, mRNA bioconjugate, small molecule, or compound of the present disclosure include chromatography (e.g., high-performance liquid chromatography, gas chromatography, etc.), nuclear magnetic resonance (NMR) spectroscopy, and combinations thereof. Analytical methods to determine the enantiomeric excess of an oligonucleotide bioconjugate, mRNA bioconjugate, small molecule, or compound of the present disclosure include chiral chromatography, polarimetry, NMR spectroscopy, and combinations thereof. In an aspect, an oligonucleotide bioconjugate, mRNA bioconjugate, small molecule, or compound of the present disclosure has an >85% diastereomeric excess and an >85% enantiomeric excess. In an aspect, an oligonucleotide bioconjugate, mRNA bioconjugate, small molecule, or compound of the present disclosure has a >90% diastereomeric excess and an >85% enantiomeric excess. In an aspect, an oligonucleotide bioconjugate, mRNA bioconjugate, small molecule, or compound of the present disclosure has an >85% diastereomeric excess and a >90% enantiomeric excess. In an aspect, an oligonucleotide bioconjugate, mRNA bioconjugate, small molecule, or compound of the present disclosure has a >90% diastereomeric excess and a >90% enantiomeric excess. In an aspect, an oligonucleotide bioconjugate, mRNA bioconjugate, small molecule, or compound of the present disclosure has a >95% diastereomeric excess and a >90% enantiomeric excess. In an aspect, an oligonucleotide bioconjugate, mRNA bioconjugate, small molecule, or compound of the present disclosure has a >90% diastereomeric excess and a >95% enantiomeric excess. In an aspect, an oligonucleotide bioconjugate, mRNA bioconjugate, small molecule, or compound of the present disclosure has a >95% diastereomeric excess and a >95% enantiomeric excess. In an aspect, an oligonucleotide bioconjugate, mRNA bioconjugate, small molecule, or compound of the present disclosure has a >98% diastereomeric excess and a >95% enantiomeric excess. In an aspect, an oligonucleotide bioconjugate, mRNA bioconjugate, small molecule, or compound of the present disclosure has a >95% diastereomeric excess and a >98% enantiomeric excess. In an aspect, an oligonucleotide bioconjugate, mRNA bioconjugate, small molecule, or compound of the present disclosure has a >98% diastereomeric excess and a >98% enantiomeric excess. In an aspect, an oligonucleotide bioconjugate, mRNA bioconjugate, small molecule, or compound of the present disclosure has a 98% diastereomeric excess and a 98% enantiomeric excess. In an aspect, an oligonucleotide bioconjugate, mRNA bioconjugate, small molecule, or compound of the present disclosure has a 99% diastereomeric excess and a 99% enantiomeric excess.

The oligonucleotide bioconjugates and mRNA bioconjugates of the present disclosure include all pharmaceutically acceptable isotopically labeled compounds of formulae (I) and (II) where one or more atoms are replaced by atoms having the same atomic number, but an atomic mass or mass number different from the atomic mass or mass number which predominates in nature. Examples of isotopes suitable for incorporation into oligonucleotide bioconjugates or mRNA bioconjugates described herein include ²H, ³H, ¹¹C, ¹³C, ¹⁴C, ³⁶Cl, ¹⁸F, ¹²³I, ¹²⁵, ¹³N, ¹⁵N, ¹⁵, ¹⁷, ¹⁸Q, ³²P, and ³⁵S. Isotopically labeled compounds of formulae (I) and (II) can be prepared by conventional techniques known to those skilled in the art.

The oligonucleotide bioconjugates, mRNA bioconjugates, small molecules, and compounds of the present disclosure may be stereochemically enriched. Conventional techniques for the preparation and isolation of individual enantiomers include chiral synthesis from a suitable optically pure precursor, and resolution of the racemate (or the racemate of a salt or a derivative, using, for example, chiral high pressure liquid chromatography (HPLC). Alternatively, the racemate may be reacted or coordinated with a suitable optically active compound and then separated by chromatography or fractional crystallization. The stereochemical purities of an oligonucleotide bioconjugate, mRNA bioconjugate, small molecule, or compound of the present disclosure may be determined by standard analytical methods. Analytical methods to determine the diastereomeric excess of an oligonucleotide bioconjugate, mRNA bioconjugate, small molecule, or compound of the present disclosure include chromatography (e.g., high-performance liquid chromatography, gas chromatography, etc.), nuclear magnetic resonance (NMR) spectroscopy, and combinations thereof. Analytical methods to determine the enantiomeric excess of an oligonucleotide bioconjugate, mRNA bioconjugate, small molecule, or compound of the present disclosure include chiral chromatography, polarimetry, NMR spectroscopy, and combinations thereof. In an aspect, an oligonucleotide bioconjugate, mRNA bioconjugate, small molecule, or compound of the present disclosure has an >85% diastereomeric excess and an >85% enantiomeric excess. In an aspect, an oligonucleotide bioconjugate, mRNA bioconjugate, small molecule, or compound of the present disclosure has a >90% diastereomeric excess and an >85% enantiomeric excess. In an aspect, an oligonucleotide bioconjugate, mRNA bioconjugate, small molecule, or compound of the present disclosure has an >85% diastereomeric excess and a >90% enantiomeric excess. In an aspect, an oligonucleotide bioconjugate, mRNA bioconjugate, small molecule, or compound of the present disclosure has a >90% diastereomeric excess and a >90% enantiomeric excess. In an aspect, an oligonucleotide bioconjugate, mRNA bioconjugate, small molecule, or compound of the present disclosure has a >95% diastereomeric excess and a >90% enantiomeric excess. In an aspect, an oligonucleotide bioconjugate, mRNA bioconjugate, small molecule, or compound of the present disclosure has a >90% diastereomeric excess and a >95% enantiomeric excess. In an aspect, an oligonucleotide bioconjugate, mRNA bioconjugate, small molecule, or compound of the present disclosure has a >95% diastereomeric excess and a >95% enantiomeric excess. In an aspect, an oligonucleotide bioconjugate, mRNA bioconjugate, small molecule, or compound of the present disclosure has a >98% diastereomeric excess and a >95% enantiomeric excess. In an aspect, an oligonucleotide bioconjugate, mRNA bioconjugate, small molecule, or compound of the present disclosure has a >95% diastereomeric excess and a >98% enantiomeric excess. In an aspect, an oligonucleotide bioconjugate, mRNA bioconjugate, small molecule, or compound of the present disclosure has a >98% diastereomeric excess and a >98% enantiomeric excess. In an aspect, an oligonucleotide bioconjugate, mRNA bioconjugate, small molecule, or compound of the present disclosure has a 98% diastereomeric excess and a 98% enantiomeric excess. In an aspect, an oligonucleotide bioconjugate, mRNA bioconjugate, small molecule, or compound of the present disclosure has a 99% diastereomeric excess and a 99% enantiomeric excess.

The oligonucleotide bioconjugates and mRNA bioconjugates of the present disclosure include all pharmaceutically acceptable isotopically labeled compounds of formulae (I), (II), (III), (IV), and (V) where one or more atoms are replaced by atoms having the same atomic number, but an atomic mass or mass number different from the atomic mass or mass number which predominates in nature. Examples of isotopes suitable for incorporation into oligonucleotide bioconjugates or mRNA bioconjugates described herein include ²H, ³H, ¹¹C, ¹³C, ¹⁴C, ³⁶Cl, ¹⁸F, ¹²³I, ¹²⁵1, ¹³N, ¹⁵N, ¹⁵O, ¹⁷O, ¹⁸O, ³²P, and ³⁵S. Isotopically labeled compounds of formulae (I), (II), (III), (IV), or (V) can be prepared by conventional techniques known to those skilled in the art.

In an aspect, an oligonucleotide bioconjugate or mRNA bioconjugate of formulae (I), (II), (III), (IV), or (V) as described herein has anti-cancer activity. In an aspect, an oligonucleotide bioconjugate or mRNA bioconjugate of formulae (I), (II), (III), (IV), or (V) as described herein treats cancer in a patient in need thereof. In an aspect, an oligonucleotide bioconjugate or mRNA bioconjugate of formulae (I), (II), (III), (IV), or (V) as described herein has anti-obesity activity. In an aspect, an oligonucleotide bioconjugate or mRNA bioconjugate of formulae (I), (II), (III), (IV), or (V) as described herein reduces or treats obesity in a patient in need thereof.

### C. Preparation of Oligonucleotide Bioconjugates and mRNA Bioconjugates

The skilled person will recognize that the oligonucleotide bioconjugates and mRNA bioconjugates of the present disclosure and pharmaceutical formulations thereof, may be prepared, in known manner, in a variety of ways using the common general knowledge of one skilled in the art of synthetic organic chemistry and biochemistry. The starting materials used to prepare the oligonucleotide bioconjugates and mRNA bioconjugates of the present disclosure, and pharmaceutical formulations thereof, are commercially available and may be prepared by routine methods known in the art.

The present disclosure provides for, and includes, the preparation of oligonucleotide bioconjugate precursors by, for example, the route shown in Scheme 1. A first oligonucleotide 1 comprising a 5′-end m7-G cap and a 3′-end poly-A tail is ligated to a second oligonucleotide 2 comprising a disulfide moiety by the action of a ligase, for example T4 RNA Ligase I, to form a ligated disulfide oligonucleotide 3 having one or more repeating poly-A subunits (e.g., n is an integer greater than or equal to 1). Each of the disulfides of oligonucleotide 3 are then cleaved with a reducing agent, such as dithiothreitol (DTT), to form the oligonucleotide bioconjugate precursor 4 having a 5′-end m7-G cap and a 3′-end thiol group.

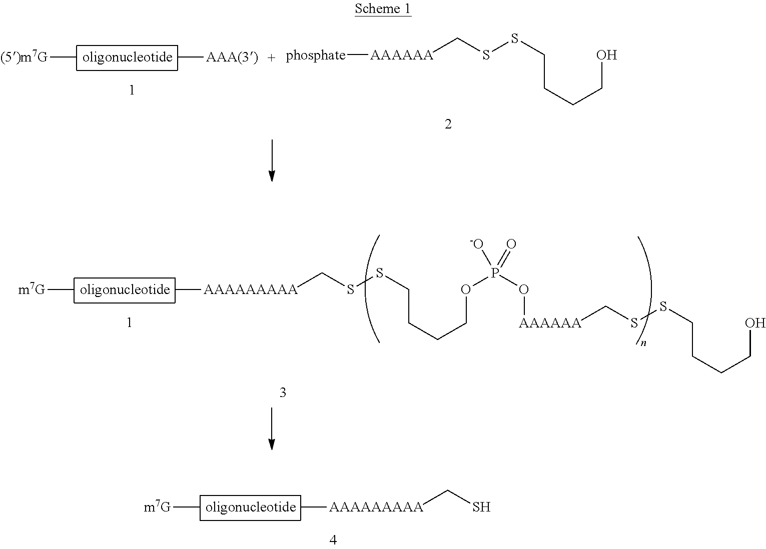

The present disclosure provides for, and includes, a method of making an oligonucleotide bioconjugate, or a pharmaceutically acceptable salt thereof, of formula (I)

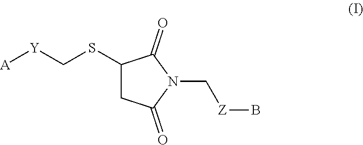

wherein
   A is an oligonucleotide having a 3′-end and a 5′-end;
   Y is a C₁-C₂₀ alkyl, wherein any one or more —CH₂— groups of the C₁-C₂₀ alkyl is each optionally replaced independently with —C(═O)—, —CF₂—, or a heteroatomic moiety selected from the group consisting of —O—, —S—, —NH—, and —N(C₁-C₆ alkyl)-, wherein any one or more —CH₃ groups of the C₁-C₂₀ alkyl is each optionally replaced independently with —CF₃, —CF₂H, —CH₂F, or a heteroatomic moiety selected from the group consisting of —NH(C₁-C₆ alkyl), —N(C₁-C₆ alkyl)₂, —O(C₁-C₆ alkyl), and —S(C₁-C₆ alkyl), and wherein any two heteroatomic moieties are separated from one another by at least two carbon atoms;
   Z is a C₁-C₂₀ alkyl, wherein any one or more —CH₂— groups of the C₁-C₂₀ alkyl is each optionally replaced independently with —C(═O)—, —CF₂—, or a heteroatomic moiety selected from the group consisting of —O—, —S—, —NH—, and —N(C₁-C₆ alkyl)-, wherein any one or more —CH₃ groups of the C₁-C₂₀ alkyl is each optionally replaced independently with —CF₃, —CF₂H, —CH₂F, or a heteroatomic moiety selected from the group consisting of —NH(C₁-C₆ alkyl), —N(C₁-C₆ alkyl)₂, —O(C₁-C₆ alkyl), and —S(C₁-C₆ alkyl), and wherein any two heteroatomic moieties are separated from one another by at least two carbon atoms; and
   B is an oligonucleotide, a polypeptide, a protein, a small molecule, a carbohydrate, a lipid, a polyethylene glycol (PEG) molecule, or a biopolymer,
wherein Y is covalently attached to the 3′-end of A through a first linkage, the method comprising a step of reacting the thiol of formula (VI) with the maleimide of formula (VII) to form the oligonucleotide bioconjugate of formula (I)

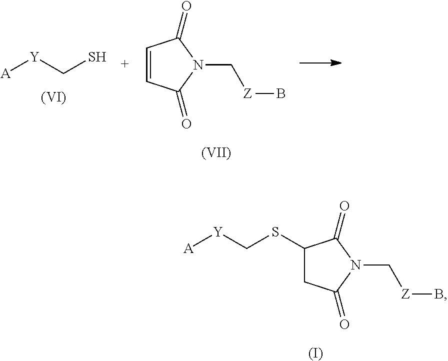

wherein A and Y of formula (VI) are defined as above for formula (I), and wherein Z and B of formula (VII) are defined as above for formula (I).

In an aspect, a first linkage is selected from the group consisting of a phosphate linkage, a phosphorothioate linkage, a phosphoramidate linkage, an amine linkage, an amide linkage, a triazole linkage, an ether linkage, and a thioether linkage. In an aspect, the first linkage is a phosphate linkage. In an aspect, the phosphate linkage between Y and A comprises a phosphate linked to the 3′-oxygen atom of a nucleotide located at the 3′-end of A. In an aspect, the first linkage is a phosphorothioate linkage. In an aspect, the first linkage is a phosphoramidate linkage. In an aspect, the first linkage is an amine linkage. In an aspect, the first linkage is an amide linkage. In an aspect, the first linkage is a triazole linkage. In an aspect, the first linkage is an ether linkage. In an aspect, the first linkage is a thioether linkage. In an aspect, the oligonucleotide bioconjugate of formula (I) comprises any of formulae (A), (B), (C), (D), (E), (F), (G), (H), and (J). In an aspect, the compound of formula (VI) comprises any of formulae (A), (B), (C), (D), (E), (F), (G), (H), and (J).

In an aspect, A comprises a poly-adenosine monophosphate region or a poly-thymidine monophosphate region. In an aspect, A comprises a poly-adenosine monophosphate region. In an aspect, A comprises a poly-thymidine monophosphate region. In an aspect, the 5′-end of A is ligated to an mRNA molecule. In an aspect, A is an mRNA molecule.

In an aspect, B is an oligonucleotide. In an aspect, B is a polypeptide. In an aspect, B is a protein. In an aspect, B is an antibody. In an aspect, B is a small molecule. In an aspect, B is a carbohydrate. In an aspect, B is cholesterol. In an aspect, B is a lipid. In an aspect, B is a PEG molecule. In an aspect, the PEG molecule is selected from the group consisting of PEG₄₀₀, PEG₁₅₀₀, PEG₂₀₀₀, PEG₃₃₅₀, PEG₄₀₀₀, PEG₆₀₀₀, PEG₈₀₀₀, PEG₁₀₀₀₀, PEG₁₅₀₀₀, PEG₂₀₀₀₀, and PEG₄₀₀₀₀. In an aspect, the PEG molecule is PEG₁₅₀₀. In an aspect, the PEG molecule is PEG₁₀₀₀₀. In an aspect, the PEG molecule is PEG₂₀₀₀₀. In an aspect, B is a biopolymer. In an aspect, the biopolymer is selected from the group consisting of proline/alanine/serine (PAS), XTEN, polysarcosine (pSar), polysaccharide, polyvinylpyrrolidone (PVP), polyglutamic acid (PGA), poly(hydroxyethyl-1-asparagine) (PHEA), poly(hydroxyethyl-1-glutamine) (PHEG), and poly(thioglycidyl glycerol) (PTTG). In an aspect, the biopolymer is PAS. In an aspect, the biopolymer is XTEN. In an aspect, the biopolymer is pSar. In an aspect, the biopolymer is PVP. In an aspect, the biopolymer is PGA. In an aspect, the biopolymer is PHEA. In an aspect, the biopolymer is PHEG. In an aspect, the biopolymer is PTTG.

In an aspect, B is an oligonucleotide having a 3′-end and a 5′-end. In an aspect, B comprises a poly-adenosine monophosphate region or a poly-thymidine monophosphate region. In an aspect, B comprises a poly-adenosine monophosphate region. In an aspect, B comprises a poly-thymidine monophosphate region. In an aspect, B is an mRNA molecule. In an aspect, B is a DNA molecule.

In an aspect, B is an oligonucleotide having a 3′-end and a 5′-end, and Z is covalently attached to the 3′-end of B through a second linkage selected from the group consisting of a phosphate linkage, a phosphorothioate linkage, a phosphoramidate linkage, an amine linkage, an amide linkage, a triazole linkage, an ether linkage, and a thioether linkage. In an aspect, the second linkage is a phosphate linkage. In an aspect, the phosphate linkage between Z and B comprises a phosphate linked to the 3′-oxygen atom of a nucleotide located at the 3′-end of B. In an aspect, the second linkage is a phosphorothioate linkage. In an aspect, the second linkage is a phosphoramidate linkage. In an aspect, the second linkage is an amine linkage. In an aspect, the first linkage is an amide linkage. In an aspect, the second linkage is a triazole linkage. In an aspect, the second linkage is an ether linkage. In an aspect, the second linkage is a thioether linkage. In an aspect, the oligonucleotide bioconjugate of formula (I) comprises any of formulae (K), (L), (M), (N), (O), (P), (Q), (R), and (S). In an aspect, the compound of formula (VII) comprises any of formulae (K), (L), (M), (N), (O), (P), (Q), (R), and (S).

In an aspect, Y is —CH₂—. In an aspect, Y is —CH₂CH₂—. In an aspect, Y is —CH₂CH₂CH₂—. In an aspect, Y is *—(CH₂)₂O(CH₂)₂—, wherein * indicates the attachment point to A. In an aspect, Y is *—(CH₂)₂O(CH₂)₂O(CH₂)₂—, wherein * indicates the attachment point to A. In an aspect, Y is *—(CH₂)₂S(CH₂)₂—, wherein * indicates the attachment point to A. In an aspect, Y is *—(CH₂)₂S(CH₂)₂S(CH₂)₂—, wherein * indicates the attachment point to A. In an aspect, Y is *—(CH₂)₂N(C₁-C₆ alkyl)(CH₂)₂—, wherein * indicates the attachment point to A. In an aspect, Y is *—(CH₂)₂N(C₁-C₆ alkyl)(CH₂)₂N(C₁-C₆ alkyl)(CH₂)₂—, wherein * indicates the attachment point to A. In an aspect, Y is —CH₂CH(OCH₃)CH₂—. In an aspect, Y is —CH₂CH(N(C₁-C₆ alkyl))CH₂—. In an aspect, Y comprises a polyamide moiety. In an aspect, Y comprises an acyl group. In an aspect, Y comprises an amide. In an aspect, Y comprises an ether. In an aspect, Y comprises an polyether. In an aspect, Y comprises a thioether. In an aspect, Y comprises an amine. In an aspect, Y comprises an polyamine. In an aspect, Y comprises a polyethylene glycol (PEG) moiety.

In an aspect, Z is —CH₂—. In an aspect, Z is —CH₂CH₂—. In an aspect, Z is —CH₂CH₂CH₂—. In an aspect, Z is *—(CH₂)₂O(CH₂)₂—, wherein * indicates the attachment point to B. In an aspect, Z is *—(CH₂)₂O(CH₂)₂O(CH₂)₂—, wherein * indicates the attachment point to B. In an aspect, Z is *—(CH₂)₂S(CH₂)₂—, wherein * indicates the attachment point to B. In an aspect, Z is *—(CH₂)₂S(CH₂)₂S(CH₂)₂—, wherein * indicates the attachment point to B. In an aspect, Z is *—(CH₂)₂N(C₁-C₆ alkyl)(CH₂)₂—, wherein * indicates the attachment point to B. In an aspect, Z is *—(CH₂)₂N(C₁-C₆ alkyl)(CH₂)₂N(C₁-C₆ alkyl)(CH₂)₂—, wherein * indicates the attachment point to B. In an aspect, Z is —CH₂CH(OCH₃)CH₂—. In an aspect, Z is —CH₂CH(N(C₁-C₆ alkyl))CH₂—. In an aspect, Z comprises a polyamide moiety. In an aspect, Z comprises an acyl group. In an aspect, Z comprises an amide. In an aspect, Z comprises an ether. In an aspect, Z comprises an polyether. In an aspect, Z comprises a thioether. In an aspect, Z comprises an amine. In an aspect, Z comprises an polyamine. In an aspect, Z comprises a polyethylene glycol (PEG) moiety.

The present disclosure provides for, and includes, a method of making an oligonucleotide bioconjugate, or a pharmaceutically acceptable salt thereof, of formula (II)

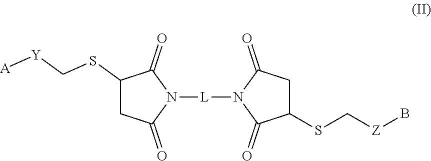

wherein
   A is an oligonucleotide having a 3′-end and a 5′-end;
   Y is a C₁-C₂₀ alkyl, wherein any one or more —CH₂— groups of the C₁-C₂₀ alkyl is each optionally replaced independently with —C(═O)—, —CF₂—, or a heteroatomic moiety selected from the group consisting of —O—, —S—, —NH—, and —N(C₁-C₆ alkyl)-, wherein any one or more —CH₃ groups of the C₁-C₂₀ alkyl is each optionally replaced independently with —CF₃, —CF₂H, —CH₂F, or a heteroatomic moiety selected from the group consisting of —NH(C₁-C₆ alkyl), —N(C₁-C₆ alkyl)₂, —O(C₁-C₆ alkyl), and —S(C₁-C₆ alkyl), and wherein any two heteroatomic moieties are separated from one another by at least two carbon atoms;
   L is a C₂-C₅₀ alkyl, C₂-C₅₀ alkenyl, C₂-C₅₀ alkynyl, C₃-C₈ cycloalkyl, aryl, heteroaryl, C₄-C₅₀ alkyl-cycloalkyl, C₇-C₅₀ alkyl-aryl, or C₆-C₅₀ alkyl-heteroaryl, wherein any one or more —CH₂— groups of L is each optionally replaced independently with —C(═O)—, —CF₂—, or a heteroatomic moiety selected from the group consisting of —O—, —S—, —NH—, and —N(C₁-C₆ alkyl)-, wherein any one or more —CH₃ groups of L is each optionally replaced independently with —CF₃, —CF₂H, —CH₂F, or a heteroatomic moiety selected from the group consisting of —NH(C₁-C₆ alkyl), —N(C₁-C₆alkyl)₂, —O(C₁-C₆ alkyl), and —S(C₁-C₆ alkyl), and wherein any two heteroatomic moieties are separated from one another by at least two carbon atoms;
   Z is a C₁-C₂₀ alkyl, wherein any one or more —CH₂— groups of the C₁-C₂₀ alkyl is each optionally replaced independently with —C(═O)—, —CF₂—, or a heteroatomic moiety selected from the group consisting of —O—, —S—, —NH—, and —N(C₁-C₆ alkyl)-, wherein any one or more —CH₃ groups of the C₁-C₂₀ alkyl is each optionally replaced independently with —CF₃, —CF₂H, —CH₂F, or a heteroatomic moiety selected from the group consisting of —NH(C₁-C₆ alkyl), —N(C₁-C₆ alkyl)₂, —O(C₁-C₆ alkyl), and —S(C₁-C₆ alkyl), and wherein any two heteroatomic moieties are separated from one another by at least two carbon atoms; and
   B is an oligonucleotide, a polypeptide, a protein, a small molecule, a lipid, a carbohydrate, a polyethylene glycol (PEG) molecule, or a biopolymer,
   wherein Y is covalently attached to the 3′-end of A through a first linkage,
the method comprising the steps of
   (i) reacting the thiol of formula (VI) with one of the two maleimides of formula (VIII) to form a compound of formula (IX)

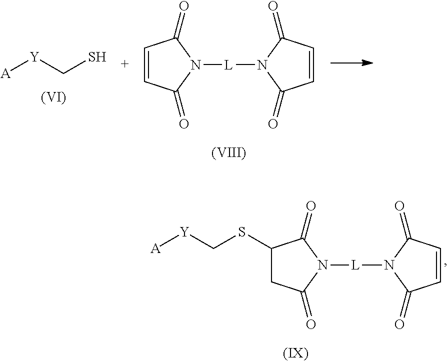

wherein A and Y of formula (VI) are defined as above for formula (II), and wherein L of formula (VIII) is defined as above for formula (II), and
(ii) reacting the maleimide of formula (IX) with the thiol of formula (X) to form the oligonucleotide bioconjugate of formula (II)

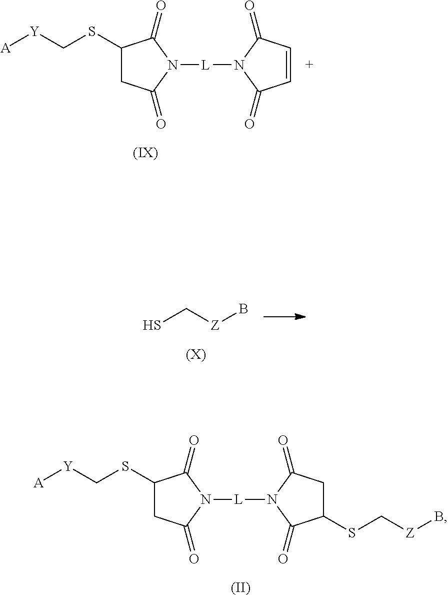

wherein Z and B of formula (X) are as defined above for formula (II).

In an aspect, a first linkage is selected from the group consisting of a phosphate linkage, a phosphorothioate linkage, a phosphoramidate linkage, an amine linkage, an amide linkage, a triazole linkage, an ether linkage, and a thioether linkage. In an aspect, the first linkage is a phosphate linkage. In an aspect, the phosphate linkage between Y and A comprises a phosphate linked to the 3′-oxygen atom of a nucleotide located at the 3′-end of A. In an aspect, the first linkage is a phosphorothioate linkage. In an aspect, the first linkage is a phosphoramidate linkage. In an aspect, the first linkage is an amine linkage. In an aspect, the first linkage is an amide linkage. In an aspect, the first linkage is a triazole linkage. In an aspect, the first linkage is an ether linkage. In an aspect, the first linkage is a thioether linkage. In an aspect, the oligonucleotide bioconjugate of formula (II) comprises any of formulae (A), (B), (C), (D), (E), (F), (G), (H), and (J). In an aspect, the compound of formula (VI) comprises any of formulae (A), (B), (C), (D), (E), (F), (G), (H), and (J). In an aspect, the compound of formula (IX) comprises any of formulae (A), (B), (C), (D), (E), (F), (G), (H), and (J).

In an aspect, A comprises a poly-adenosine monophosphate region or a poly-thymidine monophosphate region. In an aspect, A comprises a poly-adenosine monophosphate region. In an aspect, A comprises a poly-thymidine monophosphate region. In an aspect, the 5′-end of A is ligated to an mRNA molecule. In an aspect, A is an mRNA molecule.

In an aspect, B is an oligonucleotide. In an aspect, B is a polypeptide. In an aspect, B is a protein. In an aspect, B is an antibody. In an aspect, B is a small molecule. In an aspect, B is a carbohydrate. In an aspect, B is cholesterol. In an aspect, B is a lipid. In an aspect, B is a PEG molecule. In an aspect, the PEG molecule is selected from the group consisting of PEG₄₀₀, PEG₁₅₀₀, PEG₂₀₀₀, PEG₃₃₅₀, PEG₄₀₀₀, PEG₆₀₀₀, PEG₈₀₀₀, PEG₁₀₀₀₀, PEG₁₅₀₀₀, PEG₂₀₀₀₀, and PEG₄₀₀₀₀. In an aspect, the PEG molecule is PEG₁₅₀₀. In an aspect, the PEG molecule is PEG₁₀₀₀₀. In an aspect, the PEG molecule is PEG₂₀₀₀₀. In an aspect, B is a biopolymer. In an aspect, the biopolymer is selected from the group consisting of proline/alanine/serine (PAS), XTEN, polysarcosine (pSar), polysaccharide, polyvinylpyrrolidone (PVP), polyglutamic acid (PGA), poly(hydroxyethyl-1-asparagine) (P HEA), poly(hydroxyethyl-1-glutamine) (PHEG), and poly(thioglycidyl glycerol) (PTTG). In an aspect, the biopolymer is PAS. In an aspect, the biopolymer is XTEN. In an aspect, the biopolymer is pSar. In an aspect, the biopolymer is PVP. In an aspect, the biopolymer is PGA. In an aspect, the biopolymer is PHEA. In an aspect, the biopolymer is PHEG. In an aspect, the biopolymer is PTTG.

In an aspect, B is an oligonucleotide having a 3′-end and a 5′-end. In an aspect, B comprises a poly-adenosine monophosphate region or a poly-thymidine monophosphate region. In an aspect, B comprises a poly-adenosine monophosphate region. In an aspect, B comprises a poly-thymidine monophosphate region. In an aspect, B is an mRNA molecule. In an aspect, B is a DNA molecule.

In an aspect, B is an oligonucleotide having a 3′-end and a 5′-end, and Z is covalently attached to the 3′-end of B through a second linkage selected from the group consisting of a phosphate linkage, a phosphorothioate linkage, a phosphoramidate linkage, an amine linkage, an amide linkage, a triazole linkage, an ether linkage, and a thioether linkage. In an aspect, the second linkage is a phosphate linkage. In an aspect, the phosphate linkage between Z and B comprises a phosphate linked to the 3′-oxygen atom of a nucleotide located at the 3′-end of B. In an aspect, the second linkage is a phosphorothioate linkage. In an aspect, the second linkage is a phosphoramidate linkage. In an aspect, the second linkage is an amine linkage. In an aspect, the first linkage is an amide linkage. In an aspect, the second linkage is a triazole linkage. In an aspect, the second linkage is an ether linkage. In an aspect, the second linkage is a thioether linkage. In an aspect, the oligonucleotide bioconjugate of formula (II) comprises any of formulae (K), (L), (M), (N), (O), (P), (Q), (R), and (S). In an aspect, the compound of formula (X) comprises any of formulae (K), (L), (M), (N), (O), (P), (Q), (R), and (S).

In an aspect, Y is —CH₂—. In an aspect, Y is —CH₂CH₂—. In an aspect, Y is —CH₂CH₂CH₂—. In an aspect, Y is *—(CH₂)₂O(CH₂)₂—, wherein * indicates the attachment point to A. In an aspect, Y is *—(CH₂)₂O(CH₂)₂O(CH₂)₂—, wherein * indicates the attachment point to A. In an aspect, Y is *—(CH₂)₂S(CH₂)₂—, wherein * indicates the attachment point to A. In an aspect, Y is *—(CH₂)₂S(CH₂)₂S(CH₂)₂—, wherein * indicates the attachment point to A. In an aspect, Y is *—(CH₂)₂N(C₁-C₆ alkyl)(CH₂)₂—, wherein * indicates the attachment point to A. In an aspect, Y is *—(CH₂)₂N(C₁-C₆ alkyl)(CH₂)₂N(C₁-C₆ alkyl)(CH₂)₂—, wherein * indicates the attachment point to A. In an aspect, Y is —CH₂CH(OCH₃)CH₂—. In an aspect, Y is —CH₂CH(N(C₁-C₆ alkyl))CH₂—. In an aspect, Y comprises a polyamide moiety. In an aspect, Y comprises an acyl group. In an aspect, Y comprises an amide. In an aspect, Y comprises an ether. In an aspect, Y comprises an polyether. In an aspect, Y comprises a thioether. In an aspect, Y comprises an amine. In an aspect, Y comprises an polyamine. In an aspect, Y comprises a polyethylene glycol (PEG) moiety.

In an aspect, Z is —CH₂—. In an aspect, Z is —CH₂CH₂—. In an aspect, Z is —CH₂CH₂CH₂—. In an aspect, Z is *—(CH₂)₂O(CH₂)₂—, wherein * indicates the attachment point to B. In an aspect, Z is *—(CH₂)₂O(CH₂)₂O(CH₂)₂—, wherein * indicates the attachment point to B. In an aspect, Z is *—(CH₂)₂S(CH₂)₂—, wherein * indicates the attachment point to B. In an aspect, Z is *—(CH₂)₂S(CH₂)₂S(CH₂)₂—, wherein * indicates the attachment point to B. In an aspect, Z is *—(CH₂)₂N(C₁-C₆ alkyl)(CH₂)₂—, wherein * indicates the attachment point to B. In an aspect, Z is *—(CH₂)₂N(C₁-C₆ alkyl)(CH₂)₂N(C₁-C₆ alkyl)(CH₂)₂—, wherein * indicates the attachment point to B. In an aspect, Z is —CH₂CH(OCH₃)CH₂—. In an aspect, Z is —CH₂CH(N(C₁-C₆ alkyl))CH₂—. In an aspect, Z comprises a polyamide moiety. In an aspect, Z comprises an acyl group. In an aspect, Z comprises an amide. In an aspect, Z comprises an ether. In an aspect, Z comprises an polyether. In an aspect, Z comprises a thioether. In an aspect, Z comprises an amine. In an aspect, Z comprises an polyamine. In an aspect, Z comprises a polyethylene glycol (PEG) moiety.

In an aspect, L is a C₂-C₅₀ alkyl. In an aspect, L is a C₂-C₅₀ alkenyl. In an aspect, L is a C₂-C₅₀ alkynyl. In an aspect, L is a C₃-C₈ cycloalkyl. In an aspect, L comprises a polyethylene glycol (PEG) moiety. In an aspect, L is a PEG₃ moiety. In an aspect, L is a PEG₁₉ moiety. In an aspect, L comprises a polyamide moiety. In an aspect, L is a polypeptide. In an aspect, L comprises an acyl group. In an aspect, L comprises an amide. In an aspect, L comprises an ether. In an aspect, L comprises an polyether. In an aspect, L comprises a thioether. In an aspect, L comprises an amine. In an aspect, L is an aryl group. In an aspect, L comprises an aryl group. In an aspect, L comprises a disubstituted aryl group. In an aspect, L comprises a trisubstituted aryl group. In an aspect, L comprises a tetrasubstituted aryl group. In an aspect, L is a heteroaryl group. In an aspect, L comprises a heteroaryl group. In an aspect, L is a C₄-C₅₀ alkyl-cycloalkyl. In an aspect, L is a C₇-C₅₀ alkyl-aryl. In an aspect, L is a C₆-C₅₀ alkyl-heteroaryl.

The present disclosure provides for, and includes, a method of making an oligonucleotide bioconjugate, or a pharmaceutically acceptable salt thereof, of formula (III)

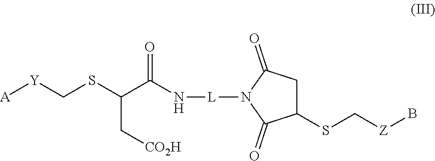

wherein
   A is an oligonucleotide having a 3′-end and a 5′-end;
   Y is a C₁-C₂₀ alkyl, wherein any one or more —CH₂— groups of the C₁-C₂₀ alkyl is each optionally replaced independently with —C(═O)—, —CF₂—, or a heteroatomic moiety selected from the group consisting of —O—, —S—, —NH—, and —N(C₁-C₆ alkyl)-, wherein any one or more —CH₃ groups of the C₁-C₂₀ alkyl is each optionally replaced independently with —CF₃, —CF₂H, —CH₂F, or a heteroatomic moiety selected from the group consisting of —NH(C₁-C₆ alkyl), —N(C₁-C₆ alkyl)₂, —O(C₁-C₆ alkyl), and —S(C₁-C₆ alkyl), and wherein any two heteroatomic moieties are separated from one another by at least two carbon atoms;
   L is a C₂-C₅₀ alkyl, C₂-C₅₀ alkenyl, C₂-C₅₀ alkynyl, C₃-C₈ cycloalkyl, aryl, heteroaryl, C₄-C₅₀ alkyl-cycloalkyl, C₇-C₅₀ alkyl-aryl, or C₆-C₅₀ alkyl-heteroaryl, wherein any one or more —CH₂— groups of L is each optionally replaced independently with —C(═O)—, —CF₂—, or a heteroatomic moiety selected from the group consisting of —O—, —S—, —NH—, and —N(C₁-C₆ alkyl)-, wherein any one or more —CH₃ groups of L is each optionally replaced independently with —CF₃, —CF₂H, —CH₂F, or a heteroatomic moiety selected from the group consisting of —NH(C₁-C₆ alkyl), —N(C₁-C₆alkyl)₂, —O(C₁-C₆ alkyl), and —S(C₁-C₆ alkyl), and wherein any two heteroatomic moieties are separated from one another by at least two carbon atoms;
   Z is a C₁-C₂₀ alkyl, wherein any one or more —CH₂— groups of the C₁-C₂₀ alkyl is each optionally replaced independently with —C(═O)—, —CF₂—, or a heteroatomic moiety selected from the group consisting of —O—, —S—, —NH—, and —N(C₁-C₆ alkyl)-, wherein any one or more —CH₃ groups of the C₁-C₂₀ alkyl is each optionally replaced independently with —CF₃, —CF₂H, —CH₂F, or a heteroatomic moiety selected from the group consisting of —NH(C₁-C₆ alkyl), —N(C₁-C₆ alkyl)₂, —O(C₁-C₆ alkyl), and —S(C₁-C₆ alkyl), and wherein any two heteroatomic moieties are separated from one another by at least two carbon atoms; and
   B is an oligonucleotide, a polypeptide, a protein, a small molecule, a lipid, a carbohydrate, a polyethylene glycol (PEG) molecule, or a biopolymer,
wherein Y is covalently attached to the 3′-end of A through a first linkage,
the method comprising the steps of
(i) reacting the thiol of formula (VI) with one of the two maleimides of formula (VIII) to form a compound of formula (IX)

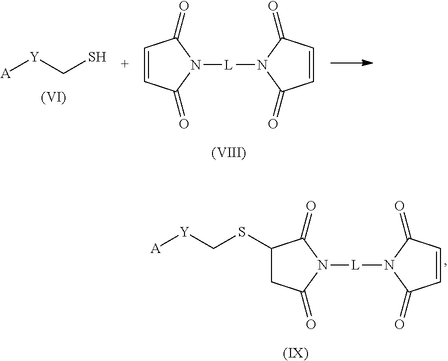

wherein A and Y of formula (VI) are defined as above for formula (III), and wherein L of formula (VIII) is defined as above for formula (III),
(ii) reacting the maleimide of formula (IX) with the thiol of formula (X) to form a compound of formula (II)

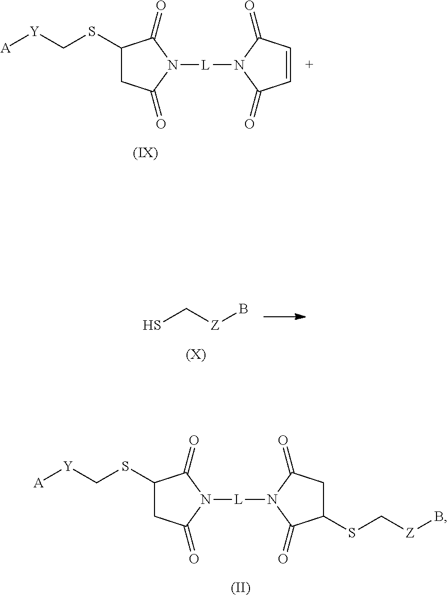

wherein Z and B of formula (X) are as defined above for formula (III), and
(iii) hydrolyzing one of the succinimides of formula (II) to form the oligonucleotide bioconjugate of formula (III)

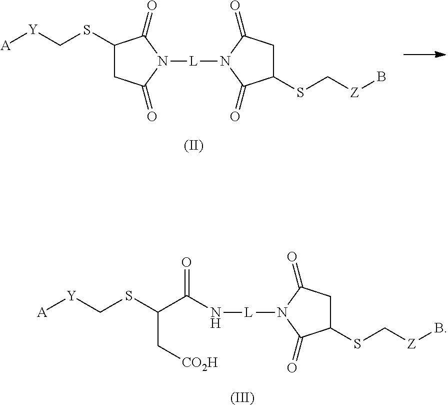

In an aspect, a first linkage is selected from the group consisting of a phosphate linkage, a phosphorothioate linkage, a phosphoramidate linkage, an amine linkage, an amide linkage, a triazole linkage, an ether linkage, and a thioether linkage. In an aspect, the first linkage is a phosphate linkage. In an aspect, the phosphate linkage between Y and A comprises a phosphate linked to the 3′-oxygen atom of a nucleotide located at the 3′-end of A. In an aspect, the first linkage is a phosphorothioate linkage. In an aspect, the first linkage is a phosphoramidate linkage. In an aspect, the first linkage is an amine linkage. In an aspect, the first linkage is an amide linkage. In an aspect, the first linkage is a triazole linkage. In an aspect, the first linkage is an ether linkage. In an aspect, the first linkage is a thioether linkage. In an aspect, the oligonucleotide bioconjugate of formula (III) comprises any of formulae (A), (B), (C), (D), (E), (F), (G), (H), and (J). In an aspect, the oligonucleotide bioconjugate of formula (II) comprises any of formulae (A), (B), (C), (D), (E), (F), (G), (H), and (J). In an aspect, the compound of formula (VI) comprises any of formulae (A), (B), (C), (D), (E), (F), (G), (H), and (J). In an aspect, the compound of formula (IX) comprises any of formulae (A), (B), (C), (D), (E), (F), (G), (H), and (J).

In an aspect, A comprises a poly-adenosine monophosphate region or a poly-thymidine monophosphate region. In an aspect, A comprises a poly-adenosine monophosphate region. In an aspect, A comprises a poly-thymidine monophosphate region. In an aspect, the 5′-end of A is ligated to an mRNA molecule. In an aspect, A is an mRNA molecule.

In an aspect, B is an oligonucleotide. In an aspect, B is a polypeptide. In an aspect, B is a protein. In an aspect, B is an antibody. In an aspect, B is a small molecule. In an aspect, B is a carbohydrate. In an aspect, B is cholesterol. In an aspect, B is a lipid. In an aspect, B is a PEG molecule. In an aspect, the PEG molecule is selected from the group consisting of PEG₄₀₀, PEG₁₅₀₀, PEG₂₀₀₀, PEG₃₃₅₀, PEG₄₀₀₀, PEG₆₀₀₀, PEG₈₀₀₀, PEG₁₀₀₀₀, PEG₁₅₀₀₀, PEG₂₀₀₀₀, and PEG₄₀₀₀₀. In an aspect, the PEG molecule is PEG₁₅₀₀. In an aspect, the PEG molecule is PEG₁₀₀₀₀. In an aspect, the PEG molecule is PEG₂₀₀₀₀. In an aspect, B is a biopolymer. In an aspect, the biopolymer is selected from the group consisting of proline/alanine/serine (PAS), XTEN, polysarcosine (pSar), polysaccharide, polyvinylpyrrolidone (PVP), polyglutamic acid (PGA), poly(hydroxyethyl-1-asparagine) (P HEA), poly(hydroxyethyl-1-glutamine) (PHEG), and poly(thioglycidyl glycerol) (PTTG). In an aspect, the biopolymer is PAS. In an aspect, the biopolymer is XTEN. In an aspect, the biopolymer is pSar. In an aspect, the biopolymer is PVP. In an aspect, the biopolymer is PGA. In an aspect, the biopolymer is PHEA. In an aspect, the biopolymer is PHEG. In an aspect, the biopolymer is PTTG.

In an aspect, B is an oligonucleotide having a 3′-end and a 5′-end. In an aspect, B comprises a poly-adenosine monophosphate region or a poly-thymidine monophosphate region. In an aspect, B comprises a poly-adenosine monophosphate region. In an aspect, B comprises a poly-thymidine monophosphate region. In an aspect, B is an mRNA molecule. In an aspect, B is a DNA molecule.

In an aspect, B is an oligonucleotide having a 3′-end and a 5′-end, and Z is covalently attached to the 3′-end of B through a second linkage selected from the group consisting of a phosphate linkage, a phosphorothioate linkage, a phosphoramidate linkage, an amine linkage, an amide linkage, a triazole linkage, an ether linkage, and a thioether linkage. In an aspect, the second linkage is a phosphate linkage. In an aspect, the phosphate linkage between Z and B comprises a phosphate linked to the 3′-oxygen atom of a nucleotide located at the 3′-end of B. In an aspect, the second linkage is a phosphorothioate linkage. In an aspect, the second linkage is a phosphoramidate linkage. In an aspect, the second linkage is an amine linkage. In an aspect, the first linkage is an amide linkage. In an aspect, the second linkage is a triazole linkage. In an aspect, the second linkage is an ether linkage. In an aspect, the second linkage is a thioether linkage. In an aspect, the oligonucleotide bioconjugate of formula (III) comprises any of formulae (K), (L), (M), (N), (O), (P), (Q), (R), and (S). In an aspect, the compound of formula (II) comprises any of formulae (K), (L), (M), (N), (O), (P), (Q), (R), and (S). In an aspect, the compound of formula (X) comprises any of formulae (K), (L), (M), (N), (O), (P), (Q), (R), and (S).

In an aspect, Y is —CH₂—. In an aspect, Y is —CH₂CH₂—. In an aspect, Y is —CH₂CH₂CH₂—. In an aspect, Y is *—(CH₂)₂O(CH₂)₂—, wherein * indicates the attachment point to A. In an aspect, Y is *—(CH₂)₂O(CH₂)₂O(CH₂)₂—, wherein * indicates the attachment point to A. In an aspect, Y is *—(CH₂)₂S(CH₂)₂—, wherein * indicates the attachment point to A. In an aspect, Y is *—(CH₂)₂S(CH₂)₂S(CH₂)₂—, wherein * indicates the attachment point to A. In an aspect, Y is *—(CH₂)₂N(C₁-C₆ alkyl)(CH₂)₂—, wherein * indicates the attachment point to A. In an aspect, Y is *—(CH₂)₂N(C₁-C₆ alkyl)(CH₂)₂N(C₁-C₆ alkyl)(CH₂)₂—, wherein * indicates the attachment point to A. In an aspect, Y is —CH₂CH(OCH₃)CH₂—. In an aspect, Y is —CH₂CH(N(C₁-C₆ alkyl))CH₂—. In an aspect, Y comprises a polyamide moiety. In an aspect, Y comprises an acyl group. In an aspect, Y comprises an amide. In an aspect, Y comprises an ether. In an aspect, Y comprises an polyether. In an aspect, Y comprises a thioether. In an aspect, Y comprises an amine. In an aspect, Y comprises an polyamine. In an aspect, Y comprises a polyethylene glycol (PEG) moiety.

In an aspect, Z is —CH₂—. In an aspect, Z is —CH₂CH₂—. In an aspect, Z is —CH₂CH₂CH₂—. In an aspect, Z is *—(CH₂)₂O(CH₂)₂—, wherein * indicates the attachment point to B. In an aspect, Z is *—(CH₂)₂O(CH₂)₂O(CH₂)₂—, wherein * indicates the attachment point to B. In an aspect, Z is *—(CH₂)₂S(CH₂)₂—, wherein * indicates the attachment point to B. In an aspect, Z is *—(CH₂)₂S(CH₂)₂S(CH₂)₂—, wherein * indicates the attachment point to B. In an aspect, Z is *—(CH₂)₂N(C₁-C₆ alkyl)(CH₂)₂—, wherein * indicates the attachment point to B. In an aspect, Z is *—(CH₂)₂N(C₁-C₆ alkyl)(CH₂)₂N(C₁-C₆ alkyl)(CH₂)₂—, wherein * indicates the attachment point to B. In an aspect, Z is —CH₂CH(OCH₃)CH₂—. In an aspect, Z is —CH₂CH(N(C₁-C₆ alkyl))CH₂—. In an aspect, Z comprises a polyamide moiety. In an aspect, Z comprises an acyl group. In an aspect, Z comprises an amide. In an aspect, Z comprises an ether. In an aspect, Z comprises an polyether. In an aspect, Z comprises a thioether. In an aspect, Z comprises an amine. In an aspect, Z comprises an polyamine. In an aspect, Z comprises a polyethylene glycol (PEG) moiety.

In an aspect, L is a C₂-C₅₀ alkyl. In an aspect, L is a C₂-C₅₀ alkenyl. In an aspect, L is a C₂-C₅₀ alkynyl. In an aspect, L is a C₃-C₈ cycloalkyl. In an aspect, L comprises a polyethylene glycol (PEG) moiety. In an aspect, L is a PEG₃ moiety. In an aspect, L is a PEG₁₉ moiety. In an aspect, L comprises a polyamide moiety. In an aspect, L is a polypeptide. In an aspect, L comprises an acyl group. In an aspect, L comprises an amide. In an aspect, L comprises an ether. In an aspect, L comprises an polyether. In an aspect, L comprises a thioether. In an aspect, L comprises an amine. In an aspect, L is an aryl group. In an aspect, L comprises an aryl group. In an aspect, L comprises a disubstituted aryl group. In an aspect, L comprises a trisubstituted aryl group. In an aspect, L comprises a tetrasubstituted aryl group. In an aspect, L is a heteroaryl group. In an aspect, L comprises a heteroaryl group. In an aspect, L is a C₄-C₅₀ alkyl-cycloalkyl. In an aspect, L is a C₇-C₅₀ alkyl-aryl. In an aspect, L is a C₆-C₅₀ alkyl-heteroaryl.

The present disclosure provides for, and includes, a method of making an oligonucleotide bioconjugate, or a pharmaceutically acceptable salt thereof, of formula (IV)

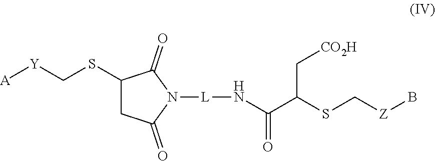

wherein
   A is an oligonucleotide having a 3′-end and a 5′-end;
   Y is a C₁-C₂₀ alkyl, wherein any one or more —CH₂— groups of the C₁-C₂₀ alkyl is each optionally replaced independently with —C(═O)—, —CF₂—, or a heteroatomic moiety selected from the group consisting of —O—, —S—, —NH—, and —N(C₁-C₆ alkyl)-, wherein any one or more —CH₃ groups of the C₁-C₂₀ alkyl is each optionally replaced independently with —CF₃, —CF₂H, —CH₂F, or a heteroatomic moiety selected from the group consisting of —NH(C₁-C₆ alkyl), —N(C₁-C₆ alkyl)₂, —O(C₁-C₆ alkyl), and —S(C₁-C₆ alkyl), and wherein any two heteroatomic moieties are separated from one another by at least two carbon atoms;
   L is a C₂-C₅₀ alkyl, C₂-C₅₀ alkenyl, C₂-C₅₀ alkynyl, C₃-C₈ cycloalkyl, aryl, heteroaryl, C₄-C₅₀ alkyl-cycloalkyl, C₇-C₅₀ alkyl-aryl, or C₆-C₅₀ alkyl-heteroaryl, wherein any one or more —CH₂— groups of L is each optionally replaced independently with —C(═O)—, —CF₂—, or a heteroatomic moiety selected from the group consisting of —O—, —S—, —NH—, and —N(C₁-C₆ alkyl)-, wherein any one or more —CH₃ groups of L is each optionally replaced independently with —CF₃, —CF₂H, —CH₂F, or a heteroatomic moiety selected from the group consisting of —NH(C₁-C₆ alkyl), —N(C₁-C₆alkyl)₂, —O(C₁-C₆ alkyl), and —S(C₁-C₆ alkyl), and wherein any two heteroatomic moieties are separated from one another by at least two carbon atoms;
   Z is a C₁-C₂₀ alkyl, wherein any one or more —CH₂— groups of the C₁-C₂₀ alkyl is each optionally replaced independently with —C(═O)—, —CF₂—, or a heteroatomic moiety selected from the group consisting of —O—, —S—, —NH—, and —N(C₁-C₆ alkyl)-, wherein any one or more —CH₃ groups of the C₁-C₂₀ alkyl is each optionally replaced independently with —CF₃, —CF₂H, —CH₂F, or a heteroatomic moiety selected from the group consisting of —NH(C₁-C₆ alkyl), —N(C₁-C₆ alkyl)₂, —O(C₁-C₆ alkyl), and —S(C₁-C₆ alkyl), and wherein any two heteroatomic moieties are separated from one another by at least two carbon atoms; and
   B is an oligonucleotide, a polypeptide, a protein, a small molecule, a lipid, a carbohydrate, a polyethylene glycol (PEG) molecule, or a biopolymer,
wherein Y is covalently attached to the 3′-end of A through a first linkage,
the method comprising the steps of
(i) reacting the thiol of formula (VI) with the maleimide of formula (VIII) to form a compound of formula (IX)

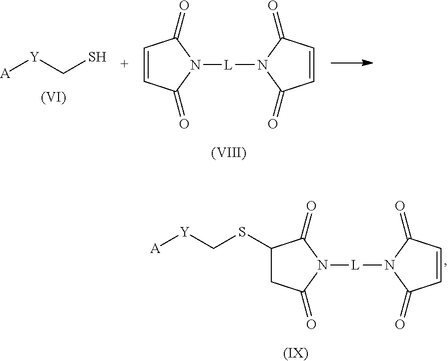

wherein A and Y of formula (VI) are defined as above for formula (IV), and wherein L of formula (VIII) is defined as above for formula (IV),
(ii) reacting the maleimide of formula (IX) with the thiol of formula (X) to form a compound of formula (II)

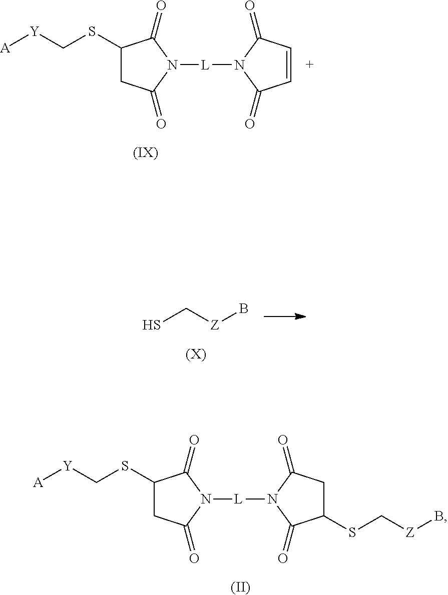

wherein Z and B of formula (X) are as defined above for formula (IV), and
(iv) hydrolyzing one of the succinimides of formula (II) to form the oligonucleotide bioconjugate of formula (IV)

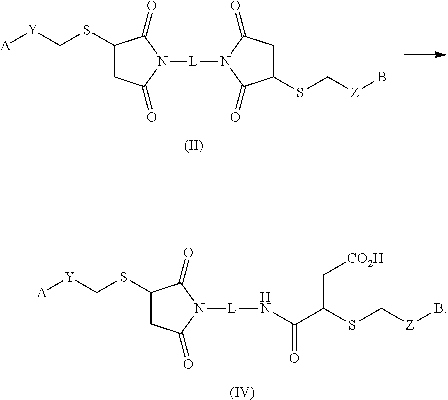

In an aspect, a first linkage is selected from the group consisting of a phosphate linkage, a phosphorothioate linkage, a phosphoramidate linkage, an amine linkage, an amide linkage, a triazole linkage, an ether linkage, and a thioether linkage. In an aspect, the first linkage is a phosphate linkage. In an aspect, the phosphate linkage between Y and A comprises a phosphate linked to the 3′-oxygen atom of a nucleotide located at the 3′-end of A. In an aspect, the first linkage is a phosphorothioate linkage. In an aspect, the first linkage is a phosphoramidate linkage. In an aspect, the first linkage is an amine linkage. In an aspect, the first linkage is an amide linkage. In an aspect, the first linkage is a triazole linkage. In an aspect, the first linkage is an ether linkage. In an aspect, the first linkage is a thioether linkage. In an aspect, the oligonucleotide bioconjugate of formula (IV) comprises any of formulae (A), (B), (C), (D), (E), (F), (G), (H), and (J). In an aspect, the oligonucleotide bioconjugate of formula (II) comprises any of formulae (A), (B), (C), (D), (E), (F), (G), (H), and (J). In an aspect, the compound of formula (VI) comprises any of formulae (A), (B), (C), (D), (E), (F), (G), (H), and (J). In an aspect, the compound of formula (IX) comprises any of formulae (A), (B), (C), (D), (E), (F), (G), (H), and (J).

In an aspect, A comprises a poly-adenosine monophosphate region or a poly-thymidine monophosphate region. In an aspect, A comprises a poly-adenosine monophosphate region. In an aspect, A comprises a poly-thymidine monophosphate region. In an aspect, the 5′-end of A is ligated to an mRNA molecule. In an aspect, A is an mRNA molecule.

In an aspect, B is an oligonucleotide. In an aspect, B is a polypeptide. In an aspect, B is a protein. In an aspect, B is an antibody. In an aspect, B is a small molecule. In an aspect, B is a carbohydrate. In an aspect, B is cholesterol. In an aspect, B is a lipid. In an aspect, B is a PEG molecule. In an aspect, the PEG molecule is selected from the group consisting of PEG₄₀₀, PEG₁₅₀₀, PEG₂₀₀₀, PEG₃₃₅₀, PEG₄₀₀₀, PEG₆₀₀₀, PEG₈₀₀₀, PEG₁₀₀₀₀, PEG₁₅₀₀₀, PEG₂₀₀₀₀, and PEG₄₀₀₀₀. In an aspect, the PEG molecule is PEG₁₅₀₀. In an aspect, the PEG molecule is PEG₁₀₀₀₀. In an aspect, the PEG molecule is PEG₂₀₀₀₀. In an aspect, B is a biopolymer. In an aspect, the biopolymer is selected from the group consisting of proline/alanine/serine (PAS), XTEN, polysarcosine (pSar), polysaccharide, polyvinylpyrrolidone (PVP), polyglutamic acid (PGA), poly(hydroxyethyl-1-asparagine) (P HEA), poly(hydroxyethyl-1-glutamine) (PHEG), and poly(thioglycidyl glycerol) (PTTG). In an aspect, the biopolymer is PAS. In an aspect, the biopolymer is XTEN. In an aspect, the biopolymer is pSar. In an aspect, the biopolymer is PVP. In an aspect, the biopolymer is PGA. In an aspect, the biopolymer is PHEA. In an aspect, the biopolymer is PHEG. In an aspect, the biopolymer is PTTG.

In an aspect, B is an oligonucleotide having a 3′-end and a 5′-end. In an aspect, B comprises a poly-adenosine monophosphate region or a poly-thymidine monophosphate region. In an aspect, B comprises a poly-adenosine monophosphate region. In an aspect, B comprises a poly-thymidine monophosphate region. In an aspect, B is an mRNA molecule. In an aspect, B is a DNA molecule.

In an aspect, B is an oligonucleotide having a 3′-end and a 5′-end, and Z is covalently attached to the 3′-end of B through a second linkage selected from the group consisting of a phosphate linkage, a phosphorothioate linkage, a phosphoramidate linkage, an amine linkage, an amide linkage, a triazole linkage, an ether linkage, and a thioether linkage. In an aspect, the second linkage is a phosphate linkage. In an aspect, the phosphate linkage between Z and B comprises a phosphate linked to the 3′-oxygen atom of a nucleotide located at the 3′-end of B. In an aspect, the second linkage is a phosphorothioate linkage. In an aspect, the second linkage is a phosphoramidate linkage. In an aspect, the second linkage is an amine linkage. In an aspect, the first linkage is an amide linkage. In an aspect, the second linkage is a triazole linkage. In an aspect, the second linkage is an ether linkage. In an aspect, the second linkage is a thioether linkage. In an aspect, the oligonucleotide bioconjugate of formula (IV) comprises any of formulae (K), (L), (M), (N), (O), (P), (Q), (R), and (S). In an aspect, the compound of formula (II) comprises any of formulae (K), (L), (M), (N), (O), (P), (Q), (R), and (S). In an aspect, the compound of formula (X) comprises any of formulae (K), (L), (M), (N), (O), (P), (Q), (R), and (S).

In an aspect, Y is —CH₂—. In an aspect, Y is —CH₂CH₂—. In an aspect, Y is —CH₂CH₂CH₂—. In an aspect, Y is *—(CH₂)₂O(CH₂)₂—, wherein * indicates the attachment point to A. In an aspect, Y is *—(CH₂)₂O(CH₂)₂O(CH₂)₂—, wherein * indicates the attachment point to A. In an aspect, Y is *—(CH₂)₂S(CH₂)₂—, wherein * indicates the attachment point to A. In an aspect, Y is *—(CH₂)₂S(CH₂)₂S(CH₂)₂—, wherein * indicates the attachment point to A. In an aspect, Y is *—(CH₂)₂N(C₁-C₆ alkyl)(CH₂)₂—, wherein * indicates the attachment point to A. In an aspect, Y is *—(CH₂)₂N(C₁-C₆ alkyl)(CH₂)₂N(C₁-C₆ alkyl)(CH₂)₂—, wherein * indicates the attachment point to A. In an aspect, Y is —CH₂CH(OCH₃)CH₂—. In an aspect, Y is —CH₂CH(N(C₁-C₆ alkyl))CH₂—. In an aspect, Y comprises a polyamide moiety. In an aspect, Y comprises an acyl group. In an aspect, Y comprises an amide. In an aspect, Y comprises an ether. In an aspect, Y comprises an polyether. In an aspect, Y comprises a thioether. In an aspect, Y comprises an amine. In an aspect, Y comprises an polyamine. In an aspect, Y comprises a polyethylene glycol (PEG) moiety.

In an aspect, Z is —CH₂—. In an aspect, Z is —CH₂CH₂—. In an aspect, Z is —CH₂CH₂CH₂—. In an aspect, Z is *—(CH₂)₂O(CH₂)₂—, wherein * indicates the attachment point to B. In an aspect, Z is *—(CH₂)₂O(CH₂)₂O(CH₂)₂—, wherein * indicates the attachment point to B. In an aspect, Z is *—(CH₂)₂S(CH₂)₂—, wherein * indicates the attachment point to B. In an aspect, Z is *—(CH₂)₂S(CH₂)₂S(CH₂)₂—, wherein * indicates the attachment point to B. In an aspect, Z is *—(CH₂)₂N(C₁-C₆ alkyl)(CH₂)₂—, wherein * indicates the attachment point to B. In an aspect, Z is *—(CH₂)₂N(C₁-C₆ alkyl)(CH₂)₂N(C₁-C₆ alkyl)(CH₂)₂—, wherein * indicates the attachment point to B. In an aspect, Z is —CH₂CH(OCH₃)CH₂—. In an aspect, Z is —CH₂CH(N(C₁-C₆ alkyl))CH₂—. In an aspect, Z comprises a polyamide moiety. In an aspect, Z comprises an acyl group. In an aspect, Z comprises an amide. In an aspect, Z comprises an ether. In an aspect, Z comprises an polyether. In an aspect, Z comprises a thioether. In an aspect, Z comprises an amine. In an aspect, Z comprises an polyamine. In an aspect, Z comprises a polyethylene glycol (PEG) moiety.

In an aspect, L is a C₂-C₅₀ alkyl. In an aspect, L is a C₂-C₅₀ alkenyl. In an aspect, L is a C₂-C₅₀ alkynyl. In an aspect, L is a C₃-C₈ cycloalkyl. In an aspect, L comprises a polyethylene glycol (PEG) moiety. In an aspect, L is a PEG₃ moiety. In an aspect, L is a PEG₁₉ moiety. In an aspect, L comprises a polyamide moiety. In an aspect, L is a polypeptide. In an aspect, L comprises an acyl group. In an aspect, L comprises an amide. In an aspect, L comprises an ether. In an aspect, L comprises an polyether. In an aspect, L comprises a thioether. In an aspect, L comprises an amine. In an aspect, L is an aryl group. In an aspect, L comprises an aryl group. In an aspect, L comprises a disubstituted aryl group. In an aspect, L comprises a trisubstituted aryl group. In an aspect, L comprises a tetrasubstituted aryl group. In an aspect, L is a heteroaryl group. In an aspect, L comprises a heteroaryl group. In an aspect, L is a C₄-C₅₀ alkyl-cycloalkyl. In an aspect, L is a C₇-C₅₀ alkyl-aryl. In an aspect, L is a C₆-C₅₀ alkyl-heteroaryl.

The present disclosure provides for, and includes, a method of making an oligonucleotide bioconjugate, or a pharmaceutically acceptable salt thereof, of formula (V)

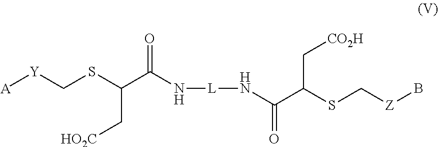

wherein
   A is an oligonucleotide having a 3′-end and a 5′-end;
   Y is a C₁-C₂₀ alkyl, wherein any one or more —CH₂— groups of the C₁-C₂₀ alkyl is each optionally replaced independently with —C(═O)—, —CF₂—, or a heteroatomic moiety selected from the group consisting of —O—, —S—, —NH—, and —N(C₁-C₆ alkyl)-, wherein any one or more —CH₃ groups of the C₁-C₂₀ alkyl is each optionally replaced independently with —CF₃, —CF₂H, —CH₂F, or a heteroatomic moiety selected from the group consisting of —NH(C₁-C₆ alkyl), —N(C₁-C₆ alkyl)₂, —O(C₁-C₆ alkyl), and —S(C₁-C₆ alkyl), and wherein any two heteroatomic moieties are separated from one another by at least two carbon atoms;
   L is a C₂-C₅₀ alkyl, C₂-C₅₀ alkenyl, C₂-C₅₀ alkynyl, C₃-C₈ cycloalkyl, aryl, heteroaryl, C₄-C₅₀ alkyl-cycloalkyl, C₇-C₅₀ alkyl-aryl, or C₆-C₅₀ alkyl-heteroaryl, wherein any one or more —CH₂— groups of L is each optionally replaced independently with —C(═O)—, —CF₂—, or a heteroatomic moiety selected from the group consisting of —O—, —S—, —NH—, and —N(C₁-C₆ alkyl)-, wherein any one or more —CH₃ groups of L is each optionally replaced independently with —CF₃, —CF₂H, —CH₂F, or a heteroatomic moiety selected from the group consisting of —NH(C₁-C₆ alkyl), —N(C₁-C₆ alkyl)₂, —O(C₁-C₆ alkyl), and —S(C₁-C₆ alkyl), and wherein any two heteroatomic moieties are separated from one another by at least two carbon atoms;
   Z is a C₁-C₂₀ alkyl, wherein any one or more —CH₂— groups of the C₁-C₂₀ alkyl is each optionally replaced independently with —C(═O)—, —CF₂—, or a heteroatomic moiety selected from the group consisting of —O—, —S—, —NH—, and —N(C₁-C₆ alkyl)-, wherein any one or more —CH₃ groups of the C₁-C₂₀ alkyl is each optionally replaced independently with —CF₃, —CF₂H, —CH₂F, or a heteroatomic moiety selected from the group consisting of —NH(C₁-C₆ alkyl), —N(C₁-C₆ alkyl)₂, —O(C₁-C₆ alkyl), and —S(C₁-C₆ alkyl), and wherein any two heteroatomic moieties are separated from one another by at least two carbon atoms; and
   B is an oligonucleotide, a polypeptide, a protein, a small molecule, a lipid, a carbohydrate, a polyethylene glycol (PEG) molecule, or a biopolymer,
wherein Y is covalently attached to the 3′-end of A through a first linkage,
the method comprising the steps of
(i) reacting the thiol of formula (VI) with one of the two maleimides of formula (VIII) to form a compound of formula (IX)

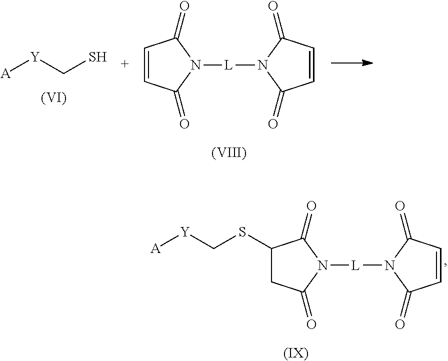

wherein A and Y of formula (VI) are defined as above for formula (V), and wherein L of formula (VIII) is defined as above for formula (V),
(ii) reacting the maleimide of formula (IX) with the thiol of formula (X) to form a compound of formula (II)

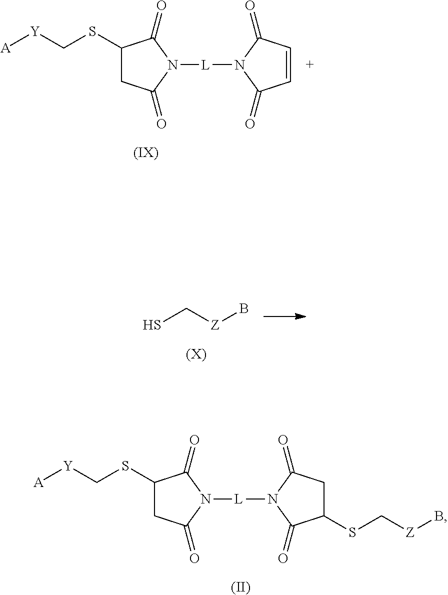

wherein Z and B of formula (X) are as defined above for formula (V), and
(iv) hydrolyzing both of the succinimides of formula (II) to form the oligonucleotide bioconjugate of formula (V)

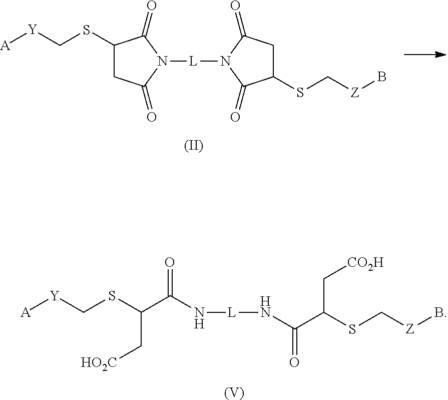

In an aspect, a first linkage is selected from the group consisting of a phosphate linkage, a phosphorothioate linkage, a phosphoramidate linkage, an amine linkage, an amide linkage, a triazole linkage, an ether linkage, and a thioether linkage. In an aspect, the first linkage is a phosphate linkage. In an aspect, the phosphate linkage between Y and A comprises a phosphate linked to the 3′-oxygen atom of a nucleotide located at the 3′-end of A. In an aspect, the first linkage is a phosphorothioate linkage. In an aspect, the first linkage is a phosphoramidate linkage. In an aspect, the first linkage is an amine linkage. In an aspect, the first linkage is an amide linkage. In an aspect, the first linkage is a triazole linkage. In an aspect, the first linkage is an ether linkage. In an aspect, the first linkage is a thioether linkage. In an aspect, the oligonucleotide bioconjugate of formula (V) comprises any of formulae (A), (B), (C), (D), (E), (F), (G), (H), and (J). In an aspect, the oligonucleotide bioconjugate of formula (II) comprises any of formulae (A), (B), (C), (D), (E), (F), (G), (H), and (J). In an aspect, the compound of formula (VI) comprises any of formulae (A), (B), (C), (D), (E), (F), (G), (H), and (J). In an aspect, the compound of formula (IX) comprises any of formulae (A), (B), (C), (D), (E), (F), (G), (H), and (J).

In an aspect, A comprises a poly-adenosine monophosphate region or a poly-thymidine monophosphate region. In an aspect, A comprises a poly-adenosine monophosphate region. In an aspect, A comprises a poly-thymidine monophosphate region. In an aspect, the 5′-end of A is ligated to an mRNA molecule. In an aspect, A is an mRNA molecule.

In an aspect, B is an oligonucleotide. In an aspect, B is a polypeptide. In an aspect, B is a protein. In an aspect, B is an antibody. In an aspect, B is a small molecule. In an aspect, B is a carbohydrate. In an aspect, B is cholesterol. In an aspect, B is a lipid. In an aspect, B is a PEG molecule. In an aspect, the PEG molecule is selected from the group consisting of PEG₄₀₀, PEG₁₅₀₀, PEG₂₀₀₀, PEG₃₃₅₀, PEG₄₀₀₀, PEG₆₀₀₀, PEG₈₀₀₀, PEG₁₀₀₀₀, PEG₁₅₀₀₀, PEG₂₀₀₀₀, and PEG₄₀₀₀₀. In an aspect, the PEG molecule is PEG₁₅₀₀. In an aspect, the PEG molecule is PEG₁₀₀₀₀. In an aspect, the PEG molecule is PEG₂₀₀₀₀. In an aspect, B is a biopolymer. In an aspect, the biopolymer is selected from the group consisting of proline/alanine/serine (PAS), XTEN, polysarcosine (pSar), polysaccharide, polyvinylpyrrolidone (PVP), polyglutamic acid (PGA), poly(hydroxyethyl-1-asparagine) (P HEA), poly(hydroxyethyl-1-glutamine) (PHEG), and poly(thioglycidyl glycerol) (PTTG). In an aspect, the biopolymer is PAS. In an aspect, the biopolymer is XTEN. In an aspect, the biopolymer is pSar. In an aspect, the biopolymer is PVP. In an aspect, the biopolymer is PGA. In an aspect, the biopolymer is PHEA. In an aspect, the biopolymer is PHEG. In an aspect, the biopolymer is PTTG.

In an aspect, B is an oligonucleotide having a 3′-end and a 5′-end. In an aspect, B comprises a poly-adenosine monophosphate region or a poly-thymidine monophosphate region. In an aspect, B comprises a poly-adenosine monophosphate region. In an aspect, B comprises a poly-thymidine monophosphate region. In an aspect, B is an mRNA molecule. In an aspect, B is a DNA molecule.

In an aspect, B is an oligonucleotide having a 3′-end and a 5′-end, and Z is covalently attached to the 3′-end of B through a second linkage selected from the group consisting of a phosphate linkage, a phosphorothioate linkage, a phosphoramidate linkage, an amine linkage, an amide linkage, a triazole linkage, an ether linkage, and a thioether linkage. In an aspect, the second linkage is a phosphate linkage. In an aspect, the phosphate linkage between Z and B comprises a phosphate linked to the 3′-oxygen atom of a nucleotide located at the 3′-end of B. In an aspect, the second linkage is a phosphorothioate linkage. In an aspect, the second linkage is a phosphoramidate linkage. In an aspect, the second linkage is an amine linkage. In an aspect, the first linkage is an amide linkage. In an aspect, the second linkage is a triazole linkage. In an aspect, the second linkage is an ether linkage. In an aspect, the second linkage is a thioether linkage. In an aspect, the oligonucleotide bioconjugate of formula (V) comprises any of formulae (K), (L), (M), (N), (O), (P), (Q), (R), and (S). In an aspect, the compound of formula (II) comprises any of formulae (K), (L), (M), (N), (O), (P), (Q), (R), and (S). In an aspect, the compound of formula (X) comprises any of formulae (K), (L), (M), (N), (O), (P), (Q), (R), and (S).

In an aspect, Y is —CH₂—. In an aspect, Y is —CH₂CH₂—. In an aspect, Y is —CH₂CH₂CH₂—. In an aspect, Y is *—(CH₂)₂O(CH₂)₂—, wherein * indicates the attachment point to A. In an aspect, Y is *—(CH₂)₂O(CH₂)₂O(CH₂)₂—, wherein * indicates the attachment point to A. In an aspect, Y is *—(CH₂)₂S(CH₂)₂—, wherein * indicates the attachment point to A. In an aspect, Y is *—(CH₂)₂S(CH₂)₂S(CH₂)₂—, wherein * indicates the attachment point to A. In an aspect, Y is *—(CH₂)₂N(C₁-C₆ alkyl)(CH₂)₂—, wherein * indicates the attachment point to A. In an aspect, Y is *—(CH₂)₂N(C₁-C₆ alkyl)(CH₂)₂N(C₁-C₆ alkyl)(CH₂)₂—, wherein * indicates the attachment point to A. In an aspect, Y is —CH₂CH(OCH₃)CH₂—. In an aspect, Y is —CH₂CH(N(C₁-C₆ alkyl))CH₂—. In an aspect, Y comprises a polyamide moiety. In an aspect, Y comprises an acyl group. In an aspect, Y comprises an amide. In an aspect, Y comprises an ether. In an aspect, Y comprises an polyether. In an aspect, Y comprises a thioether. In an aspect, Y comprises an amine. In an aspect, Y comprises an polyamine. In an aspect, Y comprises a polyethylene glycol (PEG) moiety.

In an aspect, Z is —CH₂—. In an aspect, Z is —CH₂CH₂—. In an aspect, Z is —CH₂CH₂CH₂—. In an aspect, Z is *—(CH₂)₂O(CH₂)₂—, wherein * indicates the attachment point to B. In an aspect, Z is *—(CH₂)₂O(CH₂)₂O(CH₂)₂—, wherein * indicates the attachment point to B. In an aspect, Z is *—(CH₂)₂S(CH₂)₂—, wherein * indicates the attachment point to B. In an aspect, Z is *—(CH₂)₂S(CH₂)₂S(CH₂)₂—, wherein * indicates the attachment point to B. In an aspect, Z is *—(CH₂)₂N(C₁-C₆ alkyl)(CH₂)₂—, wherein * indicates the attachment point to B. In an aspect, Z is *—(CH₂)₂N(C₁-C₆ alkyl)(CH₂)₂N(C₁-C₆ alkyl)(CH₂)₂—, wherein * indicates the attachment point to B. In an aspect, Z is —CH₂CH(OCH₃)CH₂—. In an aspect, Z is —CH₂CH(N(C₁-C₆ alkyl))CH₂—. In an aspect, Z comprises a polyamide moiety. In an aspect, Z comprises an acyl group. In an aspect, Z comprises an amide. In an aspect, Z comprises an ether. In an aspect, Z comprises an polyether. In an aspect, Z comprises a thioether. In an aspect, Z comprises an amine. In an aspect, Z comprises an polyamine. In an aspect, Z comprises a polyethylene glycol (PEG) moiety.

In an aspect, L is a C₂-C₅₀ alkyl. In an aspect, L is a C₂-C₅₀ alkenyl. In an aspect, L is a C₂-C₅₀ alkynyl. In an aspect, L is a C₃-C₈ cycloalkyl. In an aspect, L comprises a polyethylene glycol (PEG) moiety. In an aspect, L is a PEG₃ moiety. In an aspect, L is a PEG₁₉ moiety. In an aspect, L comprises a polyamide moiety. In an aspect, L is a polypeptide. In an aspect, L comprises an acyl group. In an aspect, L comprises an amide. In an aspect, L comprises an ether. In an aspect, L comprises an polyether. In an aspect, L comprises a thioether. In an aspect, L comprises an amine. In an aspect, L is an aryl group. In an aspect, L comprises an aryl group. In an aspect, L comprises a disubstituted aryl group. In an aspect, L comprises a trisubstituted aryl group. In an aspect, L comprises a tetrasubstituted aryl group. In an aspect, L is a heteroaryl group. In an aspect, L comprises a heteroaryl group. In an aspect, L is a C₄-C₅₀ alkyl-cycloalkyl. In an aspect, L is a C₇-C₅₀ alkyl-aryl. In an aspect, L is a C₆-C₅₀ alkyl-heteroaryl.

The skilled person will recognize that compounds used to make the oligonucleotide bioconjugates and mRNA bioconjugates of the present disclosure by the methods described herein, including compounds of formulae (VI), (VIII), and (X), may be prepared, in known manner, in a variety of ways.

### D. Pharmaceutical Formulations and Pharmaceutical Compositions

Provided herein are pharmaceutical formulations comprising an oligonucleotide bioconjugate or mRNA bioconjugate of formula (I) or a pharmaceutically acceptable salt thereof, and a pharmaceutically acceptable carrier, excipient, or diluent. Also provided herein are pharmaceutical compositions comprising an oligonucleotide bioconjugate or mRNA bioconjugate of formula (I) or a pharmaceutically acceptable salt thereof, and a pharmaceutically acceptable carrier, excipient, or diluent. Also provided herein are pharmaceutical formulations comprising an oligonucleotide bioconjugate or mRNA bioconjugate of formula (II) or a pharmaceutically acceptable salt thereof, and a pharmaceutically acceptable carrier, excipient, or diluent. Also provided herein are pharmaceutical formulations comprising an oligonucleotide bioconjugate or mRNA bioconjugate of formula (III) or a pharmaceutically acceptable salt thereof, and a pharmaceutically acceptable carrier, excipient, or diluent. Also provided herein are pharmaceutical formulations comprising an oligonucleotide bioconjugate or mRNA bioconjugate of formula (IV) or a pharmaceutically acceptable salt thereof, and a pharmaceutically acceptable carrier, excipient, or diluent. Also provided herein are pharmaceutical formulations comprising an oligonucleotide bioconjugate or mRNA bioconjugate of formula (V) or a pharmaceutically acceptable salt thereof, and a pharmaceutically acceptable carrier, excipient, or diluent. In an aspect, a pharmaceutical formulation provided herein comprises an effective amount of an oligonucleotide bioconjugate or mRNA bioconjugate of formula (I) and a pharmaceutically acceptable carrier, excipient, or diluent. In an aspect, a pharmaceutical formulation provided herein comprises an effective amount of an oligonucleotide bioconjugate or mRNA bioconjugate of formula (II) and a pharmaceutically acceptable carrier, excipient, or diluent. In an aspect, a pharmaceutical formulation provided herein comprises an effective amount of an oligonucleotide bioconjugate or mRNA bioconjugate of formula (III) and a pharmaceutically acceptable carrier, excipient, or diluent. In an aspect, a pharmaceutical formulation provided herein comprises an effective amount of an oligonucleotide bioconjugate or mRNA bioconjugate of formula (IV) and a pharmaceutically acceptable carrier, excipient, or diluent. In an aspect, a pharmaceutical formulation provided herein comprises an effective amount of an oligonucleotide bioconjugate or mRNA bioconjugate of formula (V) and a pharmaceutically acceptable carrier, excipient, or diluent. In an aspect, a pharmaceutical formulation provided herein comprises an effective amount of an oligonucleotide bioconjugate or mRNA bioconjugate according to the present disclosure.

A “pharmaceutically acceptable carrier, excipient, or diluent” can refer to one or more compatible solid or liquid fillers or gel substances which are suitable for use in the human body. The “compatible” herein refers to that all ingredients in a composition can be mixed with each other and can be mixed with the oligonucleotide bioconjugates or mRNA bioconjugates according to the present disclosure, while the medicinal effect of the oligonucleotide bioconjugates or mRNA bioconjugates is not significantly reduced. Some non-limiting examples of pharmaceutically acceptable carriers or diluents include cellulose and derivatives thereof (e.g., sodium carboxymethyl cellulose, sodium ethyl cellulose, and cellulose acetate), gelatin, talc, solid lubricants (e.g., stearic acid and magnesium stearate), calcium sulfate, plant oils (e.g., soybean oil, sesame oil, peanut oil, and olive oil), polyols (e.g., propylene glycol, glycerin, mannitol, and sorbitol), emulsifiers (e.g., Tween), wetting agents (e.g., sodium dodecyl sulfate), coloring agents, flavoring agents, stabilizing agents, antioxidants, preservatives, pyrogen-free water, and the like. In an aspect, the pharmaceutically acceptable carrier or diluent is sterile saline. In an aspect, the pharmaceutically acceptable carrier, excipient, or diluent is phosphate-buffered saline (PBS). In an aspect, the pharmaceutically acceptable carrier, excipient, or diluent is dextrose solution.

In an aspect, the pharmaceutically acceptable carrier, excipient, or diluent comprises a lipid-based carrier, a polymer-based carrier, or a nanocarrier. In an aspect, the pharmaceutically acceptable carrier, excipient, or diluent comprises a lipid-based carrier. In an aspect, the lipid-based carrier is a liposome, a liposome-like nanoparticle, a solid lipid nanoparticle, a nanostructured lipid carrier, a lipid-polymer hybrid nanoparticle, a nanoemulsion, an exosome, or a lipoprotein particle. See, e.g., Zhang et. al., “Lipid carriers for mRNA delivery,” Acta Pharm Sin B. 13(10):4105-4126. In an aspect, the lipid-based carrier is a liposome. In an aspect, the pharmaceutically acceptable carrier, excipient, or diluent comprises a polymer-based carrier. In an aspect, the a polymer-based carrier is polyethyleneimine (PEI), chitosan, poly(L-lysine) (PLL), poly(lactic-co-glycolic acid) (PLGA), polyamidoamine (PAMAM), a poly(β-amino) ester (PBAE), poly[2-(dimethylamino)ethyl methacrylate](PDMAEMA), or poly(ethylene glycol) (PEG). See, e.g., Mirón-Barroso et al., “Polymeric Carriers for Delivery of RNA Cancer Therapeutics,” Non-Coding RNA 8:58 (2022). In an aspect, the pharmaceutically acceptable carrier, excipient, or diluent comprises a nanocarrier. In an aspect, the nanocarrier is a polypeptide nanoparticle, a dendrimer, a lipid nanoparticle, or a self-assembled protein. See. e.g., Han et al., “Nanomaterials for Therapeutic RNA Delivery,” Matter 3:1948-1975 (2020). In an aspect, the nanocarrier is a mesoporous silica nanoparticle. In an aspect, the nanocarrier is a gold nanoparticle. In an aspect, the nanocarrier is a protamine. In an aspect, the nanocarrier is a bacterially-derived nanocell. In an aspect, the nanocarrier is a self-assembled protein. In an aspect, the self-assembled protein is a lentivirus. In an aspect, the self-assembled protein is an adenovirus.

The present disclosure contemplates that an oligonucleotide bioconjugate or mRNA bioconjugate according to the present disclosure, for example an oligonucleotide bioconjugate or mRNA bioconjugate of formulae (I), (II), (III), (IV), or (V), may be present in a pharmaceutical formulation or pharmaceutical composition as a pharmaceutically acceptable salt. For reviews on suitable salts, and pharmaceutically acceptable salts amenable for use herein, see Berge et al., “Pharmaceutical salts,” J. Pharm. Sci. 66(1):1-19 (1997); and “Handbook of Pharmaceutical Salts: Properties, selection and use”, P. H. Stahl, P. G. Vermuth, IUPAC, Wiley-VCH (2002), each of which is incorporated by reference herein in their entireties for all purposes. Without wishing to be bound by theory, a pharmaceutically acceptable salt of an oligonucleotide bioconjugate or mRNA bioconjugate described herein, for example, an oligonucleotide bioconjugate or mRNA bioconjugate of formula (I), formula (II), formula (III), formula (IV), or formula (V) may be advantageous due to one or more of its chemical or physical properties, such as stability in differing temperatures and humidities, or a desirable solubility in water, oil, or other solvent(s). In some instances, a salt may be used to aid in the isolation or purification of an oligonucleotide bioconjugate or mRNA bioconjugate of formulae (I), (II), (III), (IV), or (V).

Where an oligonucleotide bioconjugate or mRNA bioconjugate is sufficiently acidic, pharmaceutically acceptable salts include, but are not limited to, an alkali metal salt, e.g., Na or K, an alkali earth metal salt, e.g., Ca or Mg, or an organic amine salt. Where the oligonucleotide bioconjugate or mRNA bioconjugate is sufficiently basic, pharmaceutically acceptable salts include, but are not limited to, inorganic or organic acid addition salts. In an aspect, a pharmaceutically acceptable salt is a chloride salt, a bromide salt, a fluoride salt, a maleate salt, a sulfate salt, a citrate salt, a mesylate salt, a tartrate salt, a nitrate salt, a phosphate salt, an acetate salt, a trifluoroacetate salt, a gluconate salt, a fumarate salt, an oxalate salt, a sodium salt, a potassium salt, a calcium salt, a magnesium salt, or an ammonium salt. In an aspect, a pharmaceutically acceptable salt is a chloride salt. In an aspect, a pharmaceutically acceptable salt is a bromide salt. In an aspect, a pharmaceutically acceptable salt is a fluoride salt. In an aspect, a pharmaceutically acceptable salt is a maleate salt. In an aspect, a pharmaceutically acceptable salt is a sulfate salt. In an aspect, a pharmaceutically acceptable salt is a citrate salt. In an aspect, a pharmaceutically acceptable salt is a mesylate salt. In an aspect, a pharmaceutically acceptable salt is a tartrate salt. In an aspect, a pharmaceutically acceptable salt is a nitrate salt. In an aspect, a pharmaceutically acceptable salt is a phosphate salt. In an aspect, a pharmaceutically acceptable salt is an acetate salt. In an aspect, a pharmaceutically acceptable salt is a trifluoroacetate salt. In an aspect, a pharmaceutically acceptable salt is a gluconate salt. In an aspect, a pharmaceutically acceptable salt is a fumarate salt. In an aspect, a pharmaceutically acceptable salt is an oxalate salt. In an aspect, a pharmaceutically acceptable salt is a sodium salt. In an aspect, a pharmaceutically acceptable salt is a potassium salt. In an aspect, a pharmaceutically acceptable salt is a calcium salt. In an aspect, a pharmaceutically acceptable salt is a magnesium salt. In an aspect, a pharmaceutically acceptable salt is a calcium salt. In an aspect, a pharmaceutically acceptable salt is an ammonium salt.

In an aspect, an oligonucleotide bioconjugate or mRNA bioconjugate described herein may form mixtures of its salt and co-crystal forms. It should therefore be understood that the methods provided herein can employ such salt/co-crystal mixtures of, for example, the oligonucleotide bioconjugates of formulae (I), (II), (III), (IV), or (V).

Salts and co-crystals may be characterized using well known techniques, for example X-ray powder diffraction, single crystal X-ray diffraction (for example to evaluate proton position, bond lengths or bond angles), solid state NMR, (to evaluate for example, C, N or P chemical shifts) or spectroscopic techniques (to measure for example, OH, NH, or COOH signals and IR peak shifts resulting from hydrogen bonding).

In an aspect, an oligonucleotide bioconjugate or mRNA bioconjugate described herein may exist in solvated form, e.g., hydrates, including solvates of a pharmaceutically acceptable salt of an oligonucleotide bioconjugate or mRNA bioconjugate of formulae (I), (II), (III), (IV), or (V).

In an aspect, an oligonucleotide bioconjugate or mRNA bioconjugate described herein may also contain linkages (e.g., carbon-carbon bonds, carbon-nitrogen bonds such as amide bonds) wherein bond rotation is restricted about that particular linkage, e.g., restriction resulting from the presence of a ring bond or double bond. Accordingly, it is to be understood that the present disclosure encompasses all such isomers. In addition, an oligonucleotide bioconjugate or mRNA bioconjugate described herein may contain multiple tautomeric forms.

Procedures for the selection and preparation of suitable pharmaceutical formulations, depending on the route of administration, are described in, for example, “Pharmaceuticals—The Science of Dosage Form Designs”, M. E. Aulton, Churchill Livingstone, 2^{nd} Ed. 2002, incorporated by reference herein in its entirety for all purposes.

In an aspect, an oligonucleotide bioconjugate or mRNA bioconjugate according to the present disclosure is in a solid dosage form. In an aspect, a pharmaceutical formulation comprising an oligonucleotide bioconjugate or mRNA bioconjugate according to the present disclosure is in a solid dosage form. Solid dosage forms used for oral administration may include capsules, tablets, pills, powders, and granules. Among these solid dosage forms, an active an oligonucleotide bioconjugate or mRNA bioconjugate is mixed with at least one conventional inert excipient (or vehicle), such as sodium citrate or dicalcium phosphate, or mixed with any one or more of the following ingredients: (a) a filler or a compatibilizer, such as starch, lactose, sucrose, glucose, mannitol, and silicic acid; (b) a bonding agent, such as hydroxymethyl cellulose, alginate, gelatin, polyvinylpyrrolidone, sucrose, and gum arabic; (c) a moisturizer, such as glycerin; (d) a disintegrant, such as agar, calcium carbonate, potato starch or tapioca starch, alginic acid, some composite silicates, and sodium carbonate; (e) a slow solvent, such as paraffin; (f) an absorbing accelerator, such as quaternary amine compounds; (g) a wetting agent, such as cetyl alcohol and glyceryl monostearate; (h) an adsorbent, such as kaolin; and (i) a lubricant, such as talc, calcium stearate, magnesium stearate, solid polyethylene glycol, sodium dodecyl sulfate, or a mixture thereof. Dosage forms of capsules, tablets, and pills may also contain a buffer agent.

In an aspect, solid dosage forms such as tablets, sugared pills, capsules, pills, and granules may be prepared using coatings and shells, such as enteric coatings and other materials known in the art. The solid dosage forms may contain opacifiers, and moreover, active compounds or compounds in such compositions may be released in a portion of the digestive tract in a delayed manner. Non-limiting examples of embedding components that can be employed are polymeric materials and waxy materials. The active oligonucleotide bioconjugate or mRNA bioconjugate may also be formed into microcapsules with one or more of the above excipients.

In an aspect, an oligonucleotide bioconjugate or mRNA bioconjugate according to the present disclosure is in tablet form. In an aspect, a pharmaceutical formulation comprising a compound according to the present disclosure is in tablet form. In an aspect, an oligonucleotide bioconjugate or mRNA bioconjugate according to the present disclosure is in capsule form. In an aspect, a pharmaceutical formulation comprising an oligonucleotide bioconjugate or mRNA bioconjugate according to the present disclosure is in capsule form. In aspects, an oligonucleotide bioconjugate, mRNA bioconjugate, or pharmaceutical formulation in tablet form further comprises a tablet coating.

In an aspect, an oligonucleotide bioconjugate or mRNA bioconjugate according to the present disclosure is in a liquid dosage form. In an aspect, a pharmaceutical formulation comprising an oligonucleotide bioconjugate or mRNA bioconjugate according to the present disclosure is in a liquid dosage form. Liquid dosage forms for oral administration include pharmaceutically acceptable emulsions, solutions, suspensions, syrups, or tincture. In addition, a liquid dosage form may contain inert diluents conventionally employed in the art, such as water or other solvents, solubilizers, and emulsifiers, like ethanol, isopropanol, ethyl carbonate, ethyl acetate, propylene glycol, 1,3-butanediol, dimethylformamide, and oils, in particular cottonseed oil, peanut oil, corn germ oil, olive oil, castor oil, sesame oil, or a mixture of these substances.

In an aspect, liquid preparations for oral application may be in the form of syrups, solutions or suspensions. Solutions, for example, may contain the oligonucleotide bioconjugate or mRNA bioconjugate used in the methods of the present disclosure, the balance being sugar and a mixture of ethanol, water, glycerol and propylene glycol. Optionally such liquid preparations may contain coloring agents, flavoring agents, saccharine and/or carboxymethylcellulose as a thickening agent. Furthermore, other excipients known to those skilled in the art may be used when making formulations for oral use. In addition, liquid suspensions for oral application may contain a suspending agent, such as ethoxylated isostearyl alcohol, polyoxyethylene sorbitol, sorbitan ester, microcrystalline cellulose, aluminum methoxide, agar, or a mixture of these substances.

In an aspect, for oral administration, an oligonucleotide bioconjugate or mRNA bioconjugate according to the present disclosure may be admixed with one or more pharmaceutically acceptable adjuvants, diluents or carriers, for example, lactose, saccharose, sorbitol, mannitol; starch, for example, potato starch, corn starch or amylopectin; cellulose derivative; binder, for example, gelatin or polyvinylpyrrolidone; disintegrant, for example cellulose derivative, and/or lubricant, for example, magnesium stearate, calcium stearate, polyethylene glycol, wax, paraffin, and the like, and then compressed into tablets. If coated tablets are required, the cores, prepared as described above, may be coated with a suitable polymer dissolved or dispersed in water or readily volatile organic solvent(s). Alternatively, the tablet may be coated with a concentrated sugar solution which may contain, for example, gum arabic, gelatin, talcum and titanium dioxide.

In an aspect, an oral dosage form is a film-coated oral tablet. In an aspect, the dosage form is an immediate release dosage form with rapid dissolution characteristics under in vitro test conditions.

In an aspect, for the preparation of soft gelatin capsules, an oligonucleotide bioconjugate, mRNA bioconjugate, or pharmaceutical formulation of the present disclosure may be admixed with, for example, a vegetable oil or polyethylene glycol. Also, liquid or semisolid formulations of an oligonucleotide bioconjugate, mRNA bioconjugate, or pharmaceutical formulation of the present disclosure may be filled into hard gelatin capsules.

In an aspect, an oligonucleotide bioconjugate, mRNA bioconjugate, or pharmaceutical formulation of the present disclosure is provided as an oral disintegrating tablet (ODT). ODTs differ from traditional tablets in that they are designed to be dissolved on the tongue rather than swallowed whole.

In an aspect, an oligonucleotide bioconjugate, mRNA bioconjugate, or pharmaceutical formulation of the present disclosure is provided as an oral thin film or an oral disintegrating film (ODF). Without being limited by theory, such oral formulations, when placed on the tongue, hydrate via interaction with saliva, and releases the active oligonucleotide bioconjugate or mRNA bioconjugate from the dosage form. The ODF, in one aspect, contains a film-forming polymer such as hydroxypropyl methylcellulose (HPMC), hydroxypropyl cellulose (HPC), pullulan, carboxymethyl cellulose (CMC), pectin, starch, polyvinyl acetate (PVA) or sodium alginate.

In an aspect, an oligonucleotide bioconjugate, mRNA bioconjugate, or pharmaceutical formulation of the present disclosure is administered in the form of a prodrug which is broken down in the human or animal body. Examples of prodrugs include in vivo hydrolysable esters of an oligonucleotide bioconjugate or mRNA bioconjugate of formulae (I), (II), (III), (IV), or (V).

An in vivo hydrolysable (or cleavable) ester of an oligonucleotide bioconjugate or mRNA bioconjugate of the present disclosure that contains a carboxy or a hydroxy group is, for example, a pharmaceutically acceptable ester which is hydrolyzed in the human or animal body to produce the parent acid or alcohol. For examples of ester prodrugs derivatives, see: Beaumont et al., “Design of ester prodrugs to enhance oral absorption of poorly permeable compounds: challenges to the discovery scientist,” Curr. Drug. Metab. 4(6):461-485 (2003), incorporated by reference herein in its entirety for all purposes. Various other forms of prodrugs are known in the art and can be used in the methods provided herein. For examples of prodrugs, see: Rautio et al., “Prodrugs: design and clinical applications,” Nat Rev Drug Discov 7:255-270 (2008), the disclosure of which is incorporated by reference herein in its entirety for all purposes.

In an aspect, an oligonucleotide bioconjugate, mRNA bioconjugate, or pharmaceutical formulation according to the present disclosure may be entrapped in microcapsules prepared, for example, by coacervation techniques or by interfacial polymerization. For examples of such techniques, see: Remington, The Science and Practice of Pharmacy, 20^{th} Ed., Mack Publishing (2000), the disclosure of which is incorporated by reference herein in its entirety for all purposes.

In an aspect, a sustained-release preparation of an oligonucleotide bioconjugate, mRNA bioconjugate, or pharmaceutical formulation of the present disclosure is administered. Examples of sustained-release preparations include semi-permeable matrices of solid hydrophobic polymers containing the oligonucleotide bioconjugate or mRNA bioconjugate, where the matrices are in the form of shaped articles, e.g., films or microcapsules. Examples of sustained-release matrices include polyesters, hydrogels, and polylactides.

The dosage administered to a patient in need thereof will vary depending on the oligonucleotide bioconjugate, mRNA bioconjugate, or pharmaceutical formulation employed and the route of administration. Persons of ordinary skill in the art routinely determine optimum dosages, dosing methodologies and repetition rates. Optimum dosages may vary depending on the relative potency of each agent (e.g., an oligonucleotide bioconjugate or mRNAbioconjugate), and generally may be estimated based on the EC₅₀ found to be effective in in vitro and/or in vivo animal models. Dose regimen selection is also based on a variety of patient characteristics, e.g., the type, age, weight, sex, or medical condition of the patient, and the severity of the condition. Pharmaceutical compositions containing one or more oligonucleotide bioconjugates, e.g., an mRNA bioconjugate as described herein, may be given once or more daily, weekly, monthly, or even less often, or may be administered continuously for a period of time (e.g., hours, days, or weeks). In an aspect, if an oligonucleotide bioconjugate, mRNA bioconjugate, or pharmaceutical formulation of the present disclosure is administered orally, then the daily dosage used in the methods of the present disclosure may be in the range of 0.01 micrograms per kilogram body weight (g/kg) to 100 milligrams per kilogram body weight (mg/kg). In an aspect, the daily dosage used in the methods of the present disclosure may be in the range of about 1 g/kg to about 1 mg/kg. In an aspect, the daily dosage used in the methods of the present disclosure may be in the range of about 1 mg/kg to about 10 mg/kg. In an aspect, the daily dosage used in the methods of the present disclosure may be in the range of about 1 mg/kg to about 100 mg/kg.

In an aspect, a tablet for oral administration comprises between about 1 mg to about 500 mg, about 1 mg to about 100 mg, about 5 mg to about 100 mg, or about 10 mg to about 50 mg of an oligonucleotide bioconjugate, mRNA bioconjugate, or pharmaceutical formulation of the present disclosure. In an aspect, a total of one, two, three, four, five, six, seven, eight, nine, or ten tablets are administered daily. In an aspect, one tablet is administered daily. In an aspect, two tablets are administered daily. In an aspect, three tablets are administered daily. In an aspect, four tablets are administered daily. In an aspect, five tablets are administered daily. In an aspect, six tablets are administered daily. In an aspect, seven tablets are administered daily. In an aspect, eight tablets are administered daily. In an aspect, nine tablets are administered daily. In an aspect, ten tablets are administered daily.

In the methods provided herein, an oligonucleotide bioconjugate, mRNA bioconjugate, or pharmaceutical formulation of the present disclosure may be administered to a patient on an ongoing basis or for a discrete treatment period. In an aspect, the treatment period is at least one (1) month, at least three (3) months, at least six (6) months, at least twelve (12) months, at least eighteen (18) months, at least twenty-four (24) months, at least thirty (30) months, or at least thirty-six (36) months. In an aspect, the treatment period is about 4 years, about 5 years, about 6 years, about 7 years, about 8 years, about 9 years, about 10 years, about 11 years, about 12 years, about 13 years, about 14 years, about 15 years, about 16 years, about 17 years, about 18 years, about 19 years, or about 20 years. In an aspect, the treatment period is at least about one month. In an aspect, the treatment period is at least about one year. In an aspect, the treatment period is greater than one year. In an aspect, the treatment period is between one month to one year. In an aspect, the treatment period is between one month to two years. In an aspect, the treatment period is between one month to three years. In an aspect, the treatment period is between one year to three years. In an aspect, the treatment period is between one year to 10 years. In an aspect, the treatment period is open-ended.

In an aspect, an oligonucleotide bioconjugate, mRNA bioconjugate, or pharmaceutical formulation of the present disclosure is administered once daily. In an aspect, an oligonucleotide bioconjugate, mRNA bioconjugate, or pharmaceutical formulation of the present disclosure is administered once daily as an oral composition. In an aspect, the oral composition is administered once daily in tablet form at approximately the same time every day, e.g., prior to a breakfast. In an aspect, the oral composition is administered once daily in capsule form at approximately the same time every day.

In an aspect, an oligonucleotide bioconjugate, mRNA bioconjugate, or pharmaceutical formulation of the present disclosure is administered twice daily. In an aspect, an oligonucleotide bioconjugate, mRNA bioconjugate, or pharmaceutical formulation of the present disclosure is administered twice daily as an oral composition. In an aspect, the oral composition is administered twice daily in tablet form at approximately the same times every day. In an aspect, the oral composition is administered twice daily in capsule form at approximately the same time every day.

In an aspect, an oligonucleotide bioconjugate, mRNA bioconjugate, or pharmaceutical formulation of the present disclosure is administered three times a day. In an aspect, an oligonucleotide bioconjugate, mRNA bioconjugate, or pharmaceutical formulation of the present disclosure is administered three times a day as an oral composition. In an aspect, the oral composition is administered three times a day in tablet form at approximately the same times every day. In an aspect, the oral composition is administered three times a day in capsule form at approximately the same time every day.

In an aspect, an oligonucleotide bioconjugate, mRNA bioconjugate, or pharmaceutical formulation of the present disclosure is administered 1× per week, every other day, every third day, 2× per week, 3× per week, 4× per week, or 5× per week. In an aspect, an oligonucleotide bioconjugate, mRNA bioconjugate, or pharmaceutical formulation of the present disclosure is administered on an empty stomach. In an aspect, an oligonucleotide bioconjugate, mRNA bioconjugate, or pharmaceutical formulation of the present disclosure is administered before a meal. In an aspect, an oligonucleotide bioconjugate, mRNA bioconjugate, or pharmaceutical formulation of the present disclosure is administered after a meal. In aspects, an oligonucleotide bioconjugate, mRNA bioconjugate, or pharmaceutical formulation of the present disclosure is administered without food. In an aspect, an oligonucleotide bioconjugate, mRNA bioconjugate, or pharmaceutical formulation of the present disclosure is administered with food.

In an aspect, an oligonucleotide bioconjugate, mRNA bioconjugate, or pharmaceutical formulation according to the present disclosure is administered by parenterally, e.g., by injection or infusion. Compositions for parenteral injection may include a physiologically acceptable sterile aqueous or nonaqueous solution, dispersion, suspension, or emulsion, and sterile powder for re-dissolution into a sterile injectable solution or dispersion. Suitable aqueous and nonaqueous vehicles, diluents, solvents, or excipients include water, ethanol, polyol, and suitable mixtures thereof. In an aspect, parenteral administration is intravenous administration. In an aspect, parenteral administration is subcutaneous administration. In an aspect, parenteral administration is by infusion. In an aspect, parenteral administration is by intranasal administration. In an aspect, parenteral administration is intramuscular administration. In an aspect, parenteral administration is intraperitoneal administration. Suitable devices for parenteral administration include needle injectors, microneedle injectors, needle-free injectors, and infusion techniques.

In an aspect, an oligonucleotide bioconjugate, mRNA bioconjugate, or pharmaceutical formulation according to the present disclosure is administered topically to the skin or mucosa, i.e., dermally or transdermally.

In an aspect, an oligonucleotide bioconjugate, mRNA bioconjugate, or pharmaceutical formulation according to the present disclosure is administered by inhalation or insufflation, including solutions and suspensions in pharmaceutically acceptable aqueous or organic solvents, or mixtures thereof, and powders. The liquid or solid compositions may contain suitable pharmaceutically acceptable excipients. In an aspect, an oligonucleotide bioconjugate, mRNA bioconjugate, or pharmaceutical formulation according to the present disclosure is administered by the oral or nasal respiratory route. In an aspect, an oligonucleotide bioconjugate or mRNA bioconjugate according to the present disclosure in a sterile pharmaceutically acceptable solvent, may be nebulized, and the nebulized solution may be inhaled directly from a nebulizing device. Solution, suspension, or powder compositions may be administered, preferably orally or nasally, from devices which deliver the formulation in an appropriate manner.

In an aspect, an effective amount of an oligonucleotide bioconjugate, mRNA bioconjugate of the present disclosure is between about 10 micrograms (pg) and about 250 milligrams (mg). In an aspect, the effective amount is between about 10 μg and about 100 mg. In an aspect, the effective amount is between about 50 μg and about 100 mg. In an aspect, the effective amount is between about 50 μg and about 50 mg. In an aspect, the effective amount is between about 100 μg and about 50 mg. In an aspect, the effective amount is between about 100 μg and about 25 mg. In an aspect, the effective amount is between about 100 μg and about 10 mg. In an aspect, the effective amount is about 10 μg, about 20 μg, about 50 μg, about 100 μg, about 200 μg, about 500 μg, about 750 μg, about 1 mg, about 2 mg, about 5 mg, about 10 mg, about 25 mg, about 50 mg, about 75 mg, about 100 mg, about 125 mg, about 150 mg, about 175 mg, about 200 mg, about 225 mg, or about 250 mg.

In an aspect, an oligonucleotide bioconjugate, mRNA bioconjugate according to the present disclosure by may be co-administered with a second agent. In an aspect, the second agent is an anti-cancer compound. In an aspect, the second agent is an anti-obesity compound. In an aspect, the second agent is an appetite suppressant.

In an aspect, a compound or a pharmaceutical composition according to the present disclosure is administered sequentially with a second agent. In an aspect, the second agent is an anti-cancer compound. In an aspect, the second agent is an anti-obesity compound. In an aspect, the second agent is a weight loss medication. In an aspect, the second agent is an appetite suppressant. In an aspect, a compound or a pharmaceutical composition according to the present disclosure is administered prior to administration of the second agent. In an aspect, a compound or a pharmaceutical composition according to the present disclosure is administered after the administration of the second agent.

### E. Methods and Uses

The present disclosure provides methods for using the oligonucleotide conjugates, mRNA bioconjugates, formulations, and compositions disclosed herein.

The present disclosure provides a method of co-expressing a first polypeptide and a second polypeptide in a cell, comprising a step of contacting the cell with an oligonucleotide conjugate, e.g., an mRNA bioconjugate as disclosed herein. Targeted expression of polypeptides in a cell can be used to introduce polypeptides for therapeutic and other uses. Sometimes, expression of more than one type of polypeptide is desired. The present disclosure advantageously provide methods for co-expression of more than one type of polypeptide (e.g., a first and a second polypeptide), using a single oligonucleotide conjugate. In an aspect, the method for co-expression of a first and a second polypeptide uses an mRNA bioconjugate having a formula (I), (II), (III), (IV) or (V), wherein A is a first mRNA encoding a first polypeptide and B is a second mRNA encoding a second polypeptide. In an aspect, the first polypeptide is identical to the second polypeptide. In an aspect, the first polypeptide is different from the second polypeptide. In an aspect, contacting the cell with an oligonucleotide conjugate, e.g., an mRNA bioconjugate disclosed herein, comprises performing a method of transfection. In an aspect, a cell that has been transfected with a oligonucleotide conjugate, e.g., an mRNA bioconjugate disclosed herein, will express the first polypeptide and the second polypeptide from the mRNA bioconjugate.

As used herein, transfection is the process of introducing nucleic acids, including the oligonucleotide bioconjugates disclosed herein, into cells. General methods of transfection are known to persons of skill in the art and include, but are not limited to, chemical transfection methods, viral-based transfection methods, and physical/mechanical transfection methods. In an aspect, contacting the cell with an oligonucleotide conjugate, e.g., an mRNA bioconjugate disclosed herein, comprises performing a method of chemical transfection. In an aspect, the chemical transfection method is lipid-based. In an aspect, the chemical transfection method fuses cationic lipids with the oligonucleotides disclosed herein. In an aspect, the chemical transfection method is lipofection. In an aspect, the chemical transfection method is non lipid-based. In an aspect, the chemical transfection method uses calcium phosphate. In an aspect, the chemical transfection method uses cationic polymers. In an aspect, the chemical transfection method uses dendrimers. In an aspect, the chemical transfection method uses Fugene®.

In an aspect, contacting the cell with an oligonucleotide conjugate, e.g., an mRNA bioconjugate disclosed herein, comprises performing a method of physical transfection. Without being bound by theory, physical transfection methods uses physical forces to introduce payload (e.g., the oligonucleotide bioconjugates disclosed herein) into a target cell. In an aspect, the physical transfection method is electroporation. In an aspect, the physical transfection method is sonoporation. In an aspect, the physical transfection method is optical transfection. In an aspect, the physical transfection method is microinjection. In an aspect, the physical transfection method is magnetofection. In an aspect, the physical transfection method is biolistic particle delivery. In an aspect, the physical transfection method is hydrodynamic delivery.

The present disclosure provides a method of delivering equimolar amounts of a first mRNA molecule and a second mRNA molecule to a cell, comprising a step of contacting the cell with an oligonucleotide conjugate, e.g., an mRNA bioconjugate as disclosed herein. In an aspect, the mRNA bioconjugate has formula (I), (II), (III), (IV) or (V), wherein A is a first mRNA molecule and B is a second mRNA molecule. Without being bound by theory, mRNA molecules or bioconjugates may be delivered into cells for therapeutic and other purposes. Once in the cell, the mRNA molecules or bioconjugates can become the basis for expression of therapeutic polypeptides or proteins. In some instances, delivery of more than one type of mRNA molecule or bioconjugate coding for more than one polypeptide or protein is desired. Such mRNA molecules or bioconjugates can be delivered into cells using methods of transfection or transduction described herein, or by methods known generally to the person of skill in the art. Without being bound by theory, uptake of payload is stochastic, giving rise to cell-to-cell variability in payload copy numbers. Consequently, delivery of more than one type of mRNA molecule or bioconjugate will often result in unequal numbers of each type of mRNA molecule or bioconjugate. The present disclosure advantageously provide methods for delivering equimolar amounts of a first mRNA molecule and a second mRNA molecule to a cell. In an aspect, the method of delivering equimolar amounts of a first mRNA molecule and a second mRNA molecule to a cell uses an mRNA bioconjugate disclosed herein, wherein A is the first mRNA and B is the second mRNA. In an aspect, the first mRNA molecule is identical to the second mRNA molecule. In an aspect, the first mRNA molecule is different from the second mRNA molecule. In an aspect, contacting the cell with an oligonucleotide conjugate, e.g., an mRNA bioconjugate disclosed herein, comprises performing a method of transfection.

The present disclosure provides a method of targeted therapy, comprising a step of administering to a patient in need thereof a pharmaceutical formulation comprising an oligonucleotide conjugate, e.g., an mRNA bioconjugate as disclosed herein. In an aspect, the mRNA bioconjugate has formula (I), (II), (III), (IV) or (V), wherein A is a first mRNA molecule encoding a first therapeutic polypeptide, and wherein B is a second therapeutic polypeptide, a therapeutic small molecule, or a second mRNA molecule encoding a second therapeutic polypeptide. In some cases, it is desired to target two different therapeutic molecules, also known as payloads, to a specific tumor, organ, or cell type.

The present disclosure advantageously provides methods of targeted therapy where more than one type of payload (e.g., an mRNA molecule and a polypeptide, an mRNA molecule and a small molecule, a small molecule and a polypeptide) can be simultaneously delivered to the target location. Systems of targeted delivery are known to those of skill in the art, including but not limited to, using liposomes, lipid-based nanoparticles, polymer-based nanoparticles, silica-based nanoparticles, protein-drug conjugates, carbon nanotubes, metallic nanoparticles, dendrimers, quantum dots, nanogels, and nanocrystals. These systems may target specific cell types, organs, tumors, or anatomical locations, by recognizing and binding to specific cell surface receptors on the target cells. In an aspect, the targeted therapy delivers the mRNA bioconjugate to a specific cell type, organ, tumor, or anatomical location in the patient in need thereof. In an aspect, the targeted therapy delivers the mRNA bioconjugate to a specific cell type in the patient in need thereof. In an aspect, the targeted therapy delivers the mRNA bioconjugate to a specific organ in the patient in need thereof. In an aspect, the targeted therapy delivers the mRNA bioconjugate to a specific tumor in the patient in need thereof. In an aspect, the targeted therapy delivers the mRNA bioconjugate to a specific anatomical location in the patient in need thereof.

In aspect, the targeted therapy delivers a second payload to the target location. In an aspect, the second payload is B. In an aspect, B is a second mRNA molecule encoding a second therapeutic polypeptide. In an aspect, B is a second therapeutic polypeptide. In an aspect, the first therapeutic polypeptide and the second therapeutic polypeptide are different. In an aspect, the first therapeutic polypeptide and the second therapeutic polypeptide are the same. In an aspect, the method further comprises a step of co-expressing the first and second therapeutic polypeptides. In an aspect, the first and second therapeutic polypeptides are co-expressed in the specific cell type, organ, tumor, or anatomical location to which the mRNA bioconjugate has been delivered. In an aspect, the first and second therapeutic polypeptides are co-expressed in the specific cell type to which the mRNA bioconjugate has been delivered. In an aspect, the first and second therapeutic polypeptides are co-expressed in the specific organ to which the mRNA bioconjugate has been delivered. In an aspect, the first and second therapeutic polypeptides are co-expressed in the specific tumor to which the mRNA bioconjugate has been delivered. In an aspect, the first and second therapeutic polypeptides are co-expressed in the specific anatomical location to which the mRNA bioconjugate has been delivered. In an aspect, the method further comprises a step of expressing the first therapeutic polypeptide. In an aspect, the first therapeutic polypeptide is expressed in the specific cell type, organ, tumor, or anatomical location to which the mRNA bioconjugate has been delivered. In an aspect, the first therapeutic polypeptide is expressed in the specific cell type to which the mRNA bioconjugate has been delivered. In an aspect, the first therapeutic polypeptide is expressed in the specific organ to which the mRNA bioconjugate has been delivered. In an aspect, the first therapeutic polypeptide is expressed in the specific tumor to which the mRNA bioconjugate has been delivered. In an aspect, the first therapeutic polypeptide is expressed in the specific anatomical location to which the mRNA bioconjugate has been delivered.

In an aspect, B is a therapeutic antibody. In an aspect, B is a therapeutic enzyme. In an aspect, B is a therapeutic small molecule. In an aspect, B is cleaved off from the rest of the mRNA bioconjugate after it has been delivered to the specific cell type, organ, tumor, or anatomical location.

Methods of targeting delivery of payloads are known to those skilled in the art, including but not limited to, small molecule ligands, aptamers, peptides, and antibodies that interact with specific receptors on target cells. For reviews on methods of targeted delivery, see e.g., Zhao et al., “Targeting Strategies for Tissue-Specific Drug Delivery,” Cell 181(1):151-167 (2020), which is incorporated by reference herein in its entireties for all purposes. In an aspect, the targeted therapy is targeted cancer therapy. In an aspect, the targeted therapy is targeted obesity therapy.

The present disclosure provides a method of treating, preventing, slowing the progression, or reducing the severity of cancer in a patient in need thereof, comprising a step of administering to the patient in need thereof a pharmaceutical formulation comprising an oligonucleotide conjugate, e.g., an mRNA bioconjugate as disclosed herein. The present disclosure also provides a pharmaceutical formulation comprising an oligonucleotide conjugate, e.g., an mRNA bioconjugate as disclosed herein, for use in cancer therapy in a patient in need thereof. In an aspect, the mRNA bioconjugate has formula (I), (II), (III), (IV) or (V), wherein A is a first mRNA molecule encoding a first therapeutic polypeptide, and wherein B is a second therapeutic polypeptide, a therapeutic small molecule, or a second mRNA molecule encoding a second therapeutic polypeptide. In an aspect, the first therapeutic polypeptide and the second therapeutic polypeptide are different. In an aspect, the first therapeutic polypeptide and the second therapeutic polypeptide are the same.

Without being bound by theory, cancer is a group of diseases involving abnormal cell growth with the potential to invade or spread to other parts of the body. The efficacy of cancer treatment may be different for each cancer, and the methods for measuring efficacy of treatment are known to the person of skill in the art. For example, the efficacy of a particular treatment for breast cancer may be measured by markers, such as the decrease in levels of biomarkers in the blood of the patient (e.g., cancer antigen 15-3 (CA 15-3), cancer antigen 27-29 (CA 27-29), carcinoembryonic antigen (CEA), and alpha fetoprotein (AFP)), the reduction in size of tumor measured by imaging techniques (e.g., coherence tomography (CT), positron emission tomography-CT (PET-CT), magnetic resonance imaging (MRI)), and decrease in fluorodeoxyglucose (FDG) avidity as measured by PET-CT. On the other hand, the efficacy of a particular treatment for lung cancer may be measured by markers such as the decrease in the number of lesions measured by imaging techniques (e.g., CT), the decrease in FDG avidity (maximum standardized uptake), and the size and attenuation at CT. Regardless of the method used for measuring efficacy, a measurement of the marker made before treatment is compared with a measurement of the marker made after treatment to determine efficacy.

In an aspect, the oligonucleotide conjugate, e.g., an mRNA bioconjugate as disclosed herein, is administered to a patient suffering from cancer to treat the patient. In an aspect, the marker for cancer is decreased after administration compared to before administration. In an aspect, the marker for cancer is decreased after administration by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95%. In an aspect, the marker for caner is decreased by 100% compared to before administration.

In an aspect, the oligonucleotide conjugate, e.g., an mRNA bioconjugate as disclosed herein, is administered to a patient to prevent cancer. In an aspect, the patient does not have cancer before the administration of the mRNA bioconjugate. In an aspect, the marker for cancer after the administration of the mRNA bioconjugate is not indicative of cancer. In an aspect, the patient does not have cancer after the administration of the mRNA bioconjugate. In an aspect, the marker for cancer after the administration of the mRNA bioconjugate is not indicative of cancer.

In an aspect, the oligonucleotide conjugate, e.g., an mRNA bioconjugate as disclosed herein, is administered to a patient to slow the progression of cancer in the patient. In an aspect, the marker for cancer before administration is rising at a first rate over time. In an aspect, the marker for cancer after administration is rising at a second rate over time. In an aspect, the second rate over time is lower than the first rate over time. In an aspect, the marker for cancer of the patient after treatment does not rise over time.

In an aspect, the oligonucleotide conjugate, e.g., an mRNA bioconjugate as disclosed herein, is administered to a patient to reduce the severity of cancer in the patient. In an aspect, the marker for cancer is decreased after administration compared to before administration. In an aspect, the marker for cancer is decreased after administration by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95%.

In an aspect, the first therapeutic polypeptide treats, prevents, slows the progression, or reduces the severity of the cancer in the patient in need thereof. In an aspect, the second therapeutic polypeptide treats, prevents, slows the progression, or reduces the severity of the cancer in the patient in need thereof. In an aspect, the second therapeutic antibody treats, prevents, slows the progression, or reduces the severity of the cancer in the patient in need thereof. In an aspect, the second therapeutic enzyme treats, prevents, slows the progression, or reduces the severity of the cancer in the patient in need thereof. In an aspect, the second therapeutic small molecule treats, prevents, slows the progression, or reduces the severity of the cancer in the patient in need thereof. In an aspect, the cancer is selected from the group consisting of breast cancer, lung cancer, pancreatic cancer, and colorectal cancer. In an aspect, the cancer is breast cancer. In an aspect, the cancer is lung cancer. In an aspect, the cancer is pancreatic cancer. In an aspect, the cancer is colorectal cancer. In an aspect, the patient in need thereof is a human.

The present disclosure provides a method of treating, preventing, slowing the progression, or reducing the severity of obesity in a patient in need thereof, comprising a step of administering to the patient in need thereof a pharmaceutical formulation comprising an oligonucleotide conjugate, e.g., an mRNA bioconjugate as disclosed herein. In an aspect, the mRNA bioconjugate has formula (I), (II), (III), (IV) or (V), where A is a first mRNA molecule having a 3′-end and a 5′-end, wherein A encodes a first therapeutic polypeptide, and where B is a second therapeutic polypeptide, a therapeutic small molecule, or a second mRNA molecule having a 3′-end and a 5′-end encoding a second therapeutic polypeptide, and where Y is covalently attached to the 3′-end of A through a first linkage.

Without being bound by theory, obesity is a disease or disorder characterized by excessive body fat which increases the risk of various health problems. Obesity can lead to increased risk of type 2 diabetes and heart disease, it can affect bone health and reproduction, it increases the risk of certain cancers. The efficacy of treatment can be measured by comparing the body mass index (BMI) of the patient before and after administering the mRNA bioconjugate.

In an aspect, the oligonucleotide conjugate, e.g., an mRNA bioconjugate as disclosed herein, is administered to a patient suffering from obesity to treat the patient. In an aspect, the BMI of the patient is equal to or greater than 30 before administration. In an aspect, the BMI of the patient is reduced by at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, or at least 25 after administration. In an aspect, the BMI of the patient is less than 30 after administration.

In an aspect, the oligonucleotide conjugate, e.g., an mRNA bioconjugate as disclosed herein, is administered to a patient to prevent obesity. In an aspect, the BMI of the patient is less than 30 before administration. In an aspect, the BMI of the patient is reduced by at least 1, at least 2, at least 3, at least 4, at least 5, at least 6, at least 7, at least 8, at least 9, at least 10, at least 11, at least 12, at least 13, at least 14, or at least 15 after administration. In an aspect, the BMI of the patient is not changed after administration. In an aspect, the BMI of the patient is less than 30 after administration.

In an aspect, the oligonucleotide conjugate, e.g., an mRNA bioconjugate as disclosed herein, is administered to a patient to slow the progression of obesity in the patient. In an aspect, the BMI of the patient is equal to or greater than 30 before administration. In an aspect, the BMI of the patient before administration is rising at a first rate over time. In an aspect, the BMI of the patient after administration is rising at a second rate over time. In an aspect, the second rate over time is lower than the first rate over time. In an aspect, the BMI of the patient after administration does not rise over time.

In an aspect, the oligonucleotide conjugate, e.g., an mRNA bioconjugate as disclosed herein, is administered to a patient to reduce the severity of obesity in the patient. In an aspect, the BMI of the patient is equal to or greater than 30 before administration. In an aspect, the BMI of the patient is reduced by at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, or at least 25 after administration. In an aspect, the BMI of the patient is equal to or greater than 30 after administration.

In an aspect, B is a second therapeutic polypeptide. In an aspect, B is a therapeutic antibody. In an aspect, B is a therapeutic enzyme. In an aspect, B is a therapeutic small molecule. In an aspect, the first therapeutic polypeptide and the second therapeutic polypeptide are different. In an aspect, the first therapeutic polypeptide and the second therapeutic polypeptide are the same. In an aspect, the first therapeutic polypeptide treats, prevents, slows the progression, or reduces the severity of obesity in the patient in need thereof. In an aspect, the second therapeutic polypeptide treats, prevents, slows the progression, or reduces the severity of obesity in the patient in need thereof. In an aspect, the second therapeutic antibody treats, prevents, slows the progression, or reduces the severity of obesity in the patient in need thereof. In an aspect, the second therapeutic enzyme treats, prevents, slows the progression, or reduces the severity of obesity in the patient in need thereof. In an aspect, the second therapeutic small molecule treats, prevents, slows the progression, or reduces the severity of obesity in the patient in need thereof.

The present disclosure provides a method of enzyme replacement therapy, comprising a step of administering to the patient in need thereof a pharmaceutical formulation comprising an oligonucleotide conjugate, e.g., an mRNA bioconjugate as disclosed herein. The present disclosure also provides a pharmaceutical formulation comprising an oligonucleotide conjugate, e.g., an mRNA bioconjugate as disclosed herein, for use in enzyme replacement therapy (ERT) in a patient in need thereof. In an aspect, the mRNA bioconjugate has formula (I), (II), (III), (IV) or (V), where A is a first mRNA molecule having a 3′-end and a 5′-end, wherein A encodes a first enzyme, and where B is an oligonucleotide, a polypeptide, a protein, a second enzyme, a small molecule, a carbohydrate, a lipid, a polyethylene glycol (PEG) molecule, or a biopolymer, and where Y is covalently attached to the 3′-end of A through a first linkage, and where the patient in need of enzyme replacement therapy is deficient of the first enzyme.

Without being bound by theory, enzyme replacement therapy provides replacement enzymes to patients suffering from diseases resulting in enzyme deficiencies or malfunction, including but without limitation to lysosomal storage diseases or metabolic diseases. Examples of such deficiencies or malfunction result in conditions that include, but are not limited to, Gaucher disease, Fabry disease, Pompe disease, glycogen storage disease type II, metachromatic leukodystrophy, gangliosidoses, Hunter syndrome, mucopolysaccharidoses, multiple sulfatase deficiency, corneal clouding, hepatosplenomegaly, Morquio syndrome, MPS disorders, mucolipidosis, neurologic manifestations, Sandhoff disease, sialidosis, lysosomal acid lipase deficiency, adenosine deaminase deficiency, and thrombotic thrombocytopenic purpura. The replacement enzymes can be purified human-derived or animal-derived enzymes, or can be recombinant preparations. The present disclosure provides a method of administering to the patient recombinant replacement enzymes using the mRNA bioconjugate disclosed herein, where the replacement enzymes can be encoded in A or B of the bioconjugate.

In an aspect, B is a polypeptide. In an aspect, B is a protein. In an aspect, B is a second enzyme. In an aspect, B is a small molecule. In an aspect, B is a carbohydrate. In an aspect, B is a lipid. In an aspect, B is a polyethylene glycol (PEG) molecule. In an aspect, B is a biopolymer. In an aspect, B is an oligonucleotide having a 3′-end and a 5′-end, where Z is covalently attached to the 3′-end of B through a second linkage. In an aspect, B is an mRNA molecule. In an aspect, B encodes a second enzyme. In an aspect, the patient in need of enzyme replacement therapy is deficient of the second enzyme.

In an aspect, the method of enzyme replacement therapy replenishes the first enzyme in the patient in need thereof. In an aspect, the patient in need thereof is no longer deficient of the first enzyme after the administering. In an aspect, the method of enzyme replacement therapy increases a level of the first enzyme in the patient in need thereof. In an aspect, the level of the first enzyme is increased by at least 5% after the administration. In an aspect, the level of the first enzyme is increased by at least 10% after the administration. In an aspect, the level of the first enzyme is increased by at least 20% after the administration. In an aspect, the level of the first enzyme is increased by at least 30% after the administration. In an aspect, the level of the first enzyme is increased by at least 40% after the administration. In an aspect, the level of the first enzyme is increased by at least 50% after the administration. In an aspect, the level of the first enzyme is increased by at least 75% after the administration. In an aspect, the level of the first enzyme is increased by at least 90% after the administration. In an aspect, the level of the first enzyme is increased by about 5% after the administration. In an aspect, the level of the first enzyme is increased by about 10% after the administration. In an aspect, the level of the first enzyme is increased by about 20% after the administration. In an aspect, the level of the first enzyme is increased by about 30% after the administration. In an aspect, the level of the first enzyme is increased by about 40% after the administration. In an aspect, the level of the first enzyme is increased by about 50% after the administration. In an aspect, the level of the first enzyme is increased by about 75% after the administration. In an aspect, the level of the first enzyme is increased by about 90% after the administration. In an aspect, the level of the first enzyme is increased by between about 5% to about 25% after the administration. In an aspect, the level of the first enzyme is increased by between about 5% to about 50% after the administration. In an aspect, the level of the first enzyme is increased by between about 25% to about 75% after the administration. In an aspect, the level of the first enzyme is normal after the administration. In an aspect, the method of enzyme replacement therapy replenishes the second enzyme in the patient in need thereof. In an aspect, the patient in need thereof is no longer deficient of the second enzyme after the administering. In an aspect, the method of enzyme replacement therapy increases a level of the second enzyme in the patient in need thereof. In an aspect, the level of the second enzyme is increased by at least 5% after the administration. In an aspect, the level of the second enzyme is increased by at least 10% after the administration. In an aspect, the level of the second enzyme is increased by at least 20% after the administration. In an aspect, the level of the second enzyme is increased by at least 30% after the administration. In an aspect, the level of the second enzyme is increased by at least 40% after the administration. In an aspect, the level of the second enzyme is increased by at least 50% after the administration. In an aspect, the level of the second enzyme is increased by at least 75% after the administration. In an aspect, the level of the second enzyme is increased by at least 90% after the administration. In an aspect, the level of the second enzyme is increased by about 5% after the administration. In an aspect, the level of the second enzyme is increased by about 10% after the administration. In an aspect, the level of the second enzyme is increased by about 20% after the administration. In an aspect, the level of the second enzyme is increased by about 30% after the administration. In an aspect, the level of the second enzyme is increased by about 40% after the administration. In an aspect, the level of the second enzyme is increased by about 50% after the administration. In an aspect, the level of the second enzyme is increased by about 75% after the administration. In an aspect, the level of the second enzyme is increased by about 90% after the administration. In an aspect, the level of the second enzyme is increased by between about 5% to about 25% after the administration. In an aspect, the level of the second enzyme is increased by between about 5% to about 50% after the administration. In an aspect, the level of the second enzyme is increased by between about 25% to about 75% after the administration. In an aspect, the level of the second enzyme is normal following the administration. In an aspect, the level of the second enzyme is normal after the administration.

The present disclosure provides a pharmaceutical formulation comprising an oligonucleotide conjugate, e.g., an mRNA bioconjugate as disclosed herein, for use in vaccine therapy in a patient in need thereof. In an aspect, the vaccine therapy is for vaccination against an infectious disease. In an aspect, the vaccine therapy stimulates the immune system of the patient in need thereof to destroy an infectious microorganism. In an aspect, the vaccine therapy is for vaccination against cancer. In an aspect, the vaccine therapy stimulates the immune system of the patient in need thereof to destroy a tumor. In an aspect, the patient in need thereof is a human.

The present disclosure provides a method of using an oligonucleotide conjugate, e.g., an mRNA bioconjugate as disclosed herein, to conjugate amino acids to the 3′end of mRNAs. Such amino acids can be recognition sequences that enable the mRNA bioconjugate to be recognized and modified by enzymes. Without being bound by theory, sortase from S. aureus refers to prokaryotic enzymes that recognize and cleave a C-terminal peptide motif LPXTG between the Threonine and the Glycine, and catalyzes the formation of an amide bond between the carboxyl group of Threonine and the amino-group of the oligo-glycine motif in cell-wall peptidoglycan. In an aspect, the methods disclosed herein are used to attach a peptide recognition sequence to an mRNA. The recognition sequence may be any recognition sequence known to a person of skill in the art, including but not limited to those recognized by restriction enzymes. In an aspect, the peptide recognition sequence is LPXTGXX where X represents any amino acid. In an aspect, the peptide recognition sequence is Gₙ, where n is an integer. In an aspect, methods disclosed herein are used to attach a first peptide to the 3′end of a first mRNA, and a second peptide to the 3′ end of a second mRNA. In an aspect, the first peptide is LPXTGXX where X represents any amino acid. In an aspect, the first peptide is LPETGGG. In an aspect, the first peptide is LPETGEK. In an aspect, the second peptide is Gₙ, where n is an integer. In an aspect, the second peptide is GGG. In an aspect, the second peptide is GGGG. In an aspect, a sortase enzyme recognizes the LPXTGXX attached to the 3′end of the first mRNA and the Gₙ attached to the 3′end of the second mRNA. In an aspect, the sortase enzyme joins the first and the second mRNA by their 3′ends by forming a linker between the two mRNAs with the sequence LPXTG.. In an aspect, the sortase is wild-type S. aureus sortase A. In an aspect, the sortase is a mutant S. aureus sortase A.

### F. Kits

An aspect of the present disclosure provides kits comprising an oligonucleotide bioconjugate of formulae (I), (II), (III), (IV), or (V) or a pharmaceutical formulation or pharmaceutical composition comprising an oligonucleotide bioconjugate of formulae (I), (II), (III), (IV), or (V), as provided herein, that is suitable for use in performing the methods provided herein. An aspect of the present disclosure provides kits suitable for use in performing the methods provided herein, where the kit comprises (a) an oligonucleotide bioconjugate of formulae (I), (II), (III), (IV), or (V), or a pharmaceutical formulation or pharmaceutical composition comprising an oligonucleotide bioconjugate of formulae (I), (II), (III), (IV), or (V), as provided herein, and (b) a second agent as provided herein.

A kit can further include instructions for using the kit in a diagnostic or therapeutic method, including methods to treat, prevent, slow the progression, or reduce the severity of cancer as provided herein. In an aspect, the cancer is breast cancer, lung cancer, pancreatic cancer, or colorectal cancer. A kit can also further include instructions for using the kit in diagnostic or therapeutic methods to treat, prevent, slow the progression, or reduce the severity of a metabolic and/or cardiovascular disease or disorder as provided herein. In an aspect, the metabolic and/or cardiovascular disease is obesity. A kit can also further include instructions for using the kit in targeted therapy. A kit can also further include instructions for using the kit in enzyme replacement therapy.

In an aspect, a kit contains a first dosage form comprising one or more oligonucleotide bioconjugates, or a pharmaceutical composition or formulation thereof, of the present disclosure in quantities sufficient to carry out the methods provided herein. In an aspect, a kit comprises (a) one or more oligonucleotide bioconjugates, or a pharmaceutical composition or formulation thereof, of the present disclosure in quantities sufficient to carry out the methods provided herein, and (b) a container for the dosage of the one or more oligonucleotide bioconjugates.

Having now generally described the disclosure, the same will be more readily understood through reference to the following examples that are provided by way of illustration and are not intended to be limiting of the present disclosure, unless specified.

### G. Embodiments

From the foregoing, it will be appreciated that the subject matter of the present disclosure can be embodied in various ways, which include but are not limited to the following:

Embodiment 1. An oligonucleotide bioconjugate of formula (I)

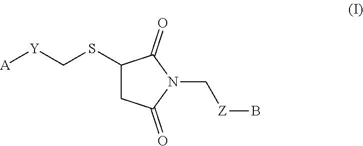

or a pharmaceutically acceptable salt thereof,
wherein
   A is an oligonucleotide having a 3′-end and a 5′-end;
   Y is a C₁-C₂₀ alkyl, wherein any one or more —CH₂— groups of the C₁-C₂₀ alkyl is each optionally replaced independently with —C(═O)—, —CF₂—, or a heteroatomic moiety selected from the group consisting of —O—, —S—, —NH—, and —N(C₁-C₆ alkyl)-, wherein any one or more —CH₃ groups of the C₁-C₂₀ alkyl is each optionally replaced independently with —CF₃, —CF₂H, —CH₂F, or a heteroatomic moiety selected from the group consisting of —NH(C₁-C₆ alkyl), —N(C₁-C₆ alkyl)₂, —O(C₁-C₆ alkyl), and —S(C₁-C₆ alkyl), and wherein any two heteroatomic moieties are separated from one another by at least two carbon atoms;
   Z is a C₁-C₂₀ alkyl, wherein any one or more —CH₂— groups of the C₁-C₂₀ alkyl is each optionally replaced independently with —C(═O)—, —CF₂—, or a heteroatomic moiety selected from the group consisting of —O—, —S—, —NH—, and —N(C₁-C₆ alkyl)-, wherein any one or more —CH₃ groups of the C₁-C₂₀ alkyl is each optionally replaced independently with —CF₃, —CF₂H, —CH₂F, or a heteroatomic moiety selected from the group consisting of —NH(C₁-C₆ alkyl), —N(C₁-C₆ alkyl)₂, —O(C₁-C₆ alkyl), and —S(C₁-C₆ alkyl), and wherein any two heteroatomic moieties are separated from one another by at least two carbon atoms; and
   B is an oligonucleotide, a polypeptide, a protein, a small molecule, a carbohydrate, a lipid, a polyethylene glycol (PEG) molecule, or a biopolymer,
wherein Y is covalently attached to the 3′-end of A through a first linkage.

Embodiment 2. The oligonucleotide bioconjugate of Embodiment 1, wherein the first linkage is selected from the group consisting of a phosphate linkage, a phosphorothioate linkage, a phosphoramidate linkage, an amine linkage, an amide linkage, a triazole linkage, an ether linkage, and a thioether linkage.

Embodiment 3. The oligonucleotide bioconjugate of Embodiment 1 or Embodiment 2, wherein the first linkage is a phosphate linkage.

Embodiment 4. The oligonucleotide bioconjugate of Embodiment 3, wherein the phosphate linkage between Y and A comprises a phosphate linked to the 3′-oxygen atom of a nucleotide located at the 3′-end of A.

Embodiment 5. The oligonucleotide bioconjugate of any one of Embodiments 1 to 4, wherein A comprises a poly-adenosine monophosphate region or a poly-thymidine monophosphate region.

Embodiment 6. The oligonucleotide bioconjugate of any one of Embodiments 1 to 5, wherein the 5′-end of A is ligated to an mRNA molecule.

Embodiment 7. The oligonucleotide bioconjugate of any one of Embodiments 1 to 4, wherein A is an mRNA molecule.

Embodiment 8. The oligonucleotide bioconjugate of any one of Embodiments 1 to 7, wherein B is an oligonucleotide having a 3′-end and a 5′-end.

Embodiment 9. The oligonucleotide bioconjugate of Embodiment 8, wherein Z is covalently attached to the 3′-end of B through a second linkage selected from the group consisting of a phosphate linkage, a phosphorothioate linkage, a phosphoramidate linkage. an amine linkage, an amide linkage, a triazole linkage, an ether linkage, and a thioether linkage.

Embodiment 10. The oligonucleotide bioconjugate of Embodiment 9, wherein the second linkage is a phosphate linkage.

Embodiment 11. The oligonucleotide bioconjugate of Embodiment 10, wherein the phosphate linkage between Z and B comprises a phosphate linked to the 3′-oxygen atom of a nucleotide located at the 3′-end of B.

Embodiment 12. The oligonucleotide bioconjugate of any one of Embodiments 8 to 11, wherein B comprises a poly-adenosine monophosphate region or a poly-thymidine monophosphate region.

Embodiment 13. The oligonucleotide bioconjugate of any one of Embodiments 8 to 21, wherein the 5′-end of B is ligated to an mRNA molecule.

Embodiment 14. The oligonucleotide bioconjugate of any one of Embodiments 8 to 11, wherein B is an mRNA molecule.

Embodiment 15. The oligonucleotide bioconjugate of any one of Embodiments 1 to 7, wherein B is a DNA molecule.

Embodiment 16. The oligonucleotide bioconjugate of any one of Embodiments 1 to 7, wherein B is a polypeptide.

Embodiment 17. The oligonucleotide bioconjugate of any one of Embodiments 1 to 7, wherein B is a protein.

Embodiment 18. The oligonucleotide bioconjugate of Embodiment 17, wherein B is an antibody.

Embodiment 19. The oligonucleotide bioconjugate of any one of Embodiments 1 to 7, wherein B is a small molecule.

Embodiment 20. The oligonucleotide bioconjugate of any one of Embodiments 1 to 7, wherein B is a carbohydrate.

Embodiment 21. The oligonucleotide bioconjugate of any one of Embodiments 1 to 7, wherein B is a lipid.

Embodiment 22. The oligonucleotide bioconjugate of any one of Embodiments 1 to 7, wherein B is a PEG molecule.

Embodiment 23. The oligonucleotide bioconjugate of Embodiment 22, wherein the PEG molecule is selected from the group consisting of PEG₄₀₀, PEG₁₅₀₀, PEG₃₃₅₀, PEG₄₀₀₀, PEG₆₀₀₀, PEG₈₀₀₀, PEG₁₀₀₀₀, PEG₁₅₀₀₀, and PEG₂₀₀₀₀.

Embodiment 24. The oligonucleotide bioconjugate of any one of Embodiments 1 to 7, wherein B is biopolymer.

Embodiment 25. The oligonucleotide bioconjugate of Embodiment 24, wherein the biopolymer is selected from the group consisting of proline/alanine/serine (PAS), XTEN, polysarcosine (pSar), polysaccharide, polyvinylpyrrolidone (PVP), polyglutamic acid (PGA), poly(hydroxyethyl-1-asparagine) (PHEA), poly(hydroxyethyl-1-glutamine) (PHEG), and poly(thioglycidyl glycerol) (PTTG).

Embodiment 26. The oligonucleotide bioconjugate of any one of Embodiments 1 to 25, wherein Y is —CH₂—.

Embodiment 27. The oligonucleotide bioconjugate of any one of Embodiments 1 to 25, wherein Y is —CH₂CH₂—.

Embodiment 28. The oligonucleotide bioconjugate of any one of Embodiments 1 to 25, wherein Y is *—(CH₂)₂O(CH₂)₂—, wherein * indicates the attachment point to A.

Embodiment 29. The oligonucleotide bioconjugate of any one of Embodiments 1 to 25, wherein Y is *—(CH₂)₂O(CH₂)₂O(CH₂)₂—, wherein * indicates the attachment point to A.

Embodiment 30. The oligonucleotide bioconjugate of any one of Embodiments 1 to 25, wherein Y is —CH₂CH(OCH₃)CH₂—.

Embodiment 31. The oligonucleotide bioconjugate of any one of Embodiments 1 to 25, wherein Y comprises a polyethylene glycol (PEG) moiety.

Embodiment 32. The oligonucleotide bioconjugate of any one of Embodiments 1 to 25, wherein Y comprises a polyamide moiety.

Embodiment 33. The oligonucleotide bioconjugate of any one of Embodiments 1 to 25, wherein Y comprises an acyl group.

Embodiment 34. The oligonucleotide bioconjugate of any one of Embodiments 1 to 33, wherein Z is —CH₂—.

Embodiment 35. The oligonucleotide bioconjugate of any one of Embodiments 1 to 33, wherein Z is —CH₂CH₂—.

Embodiment 36. The oligonucleotide bioconjugate of any one of Embodiments 1 to 33, wherein Z is —(CH₂)₂O(CH₂)₂—*, wherein * indicates the attachment point to B.

Embodiment 37. The oligonucleotide bioconjugate of any one of Embodiments 1 to 33, wherein Z is —(CH₂)₂O(CH₂)₂O(CH₂)₂—*, wherein * indicates the attachment point to B.

Embodiment 38. The oligonucleotide bioconjugate of any one of Embodiments 1 to 33, wherein Z is —CH₂CH(OCH₃)CH₂—.

Embodiment 39. The oligonucleotide bioconjugate of any one of Embodiments 1 to 33, wherein Z comprises a polyethylene glycol (PEG) moiety.

Embodiment 40. The oligonucleotide bioconjugate of any one of Embodiments 1 to 33, wherein Z comprises a polyamide moiety.

Embodiment 41. The oligonucleotide bioconjugate of any one of Embodiments 1 to 33, wherein Z comprises an acyl group.

Embodiment 42. An oligonucleotide bioconjugate of formula (II)

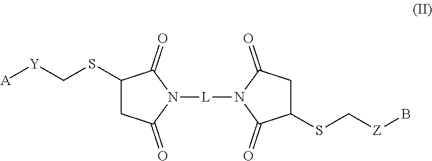

or a pharmaceutically acceptable salt thereof,
wherein
   A is an oligonucleotide having a 3′-end and a 5′-end;
   Y is a C₁-C₂₀ alkyl, wherein any one or more —CH₂— groups of the C₁-C₂₀ alkyl is each optionally replaced independently with —C(═O)—, —CF₂—, or a heteroatomic moiety selected from the group consisting of —O—, —S—, —NH—, and —N(C₁-C₆ alkyl)-, wherein any one or more —CH₃ groups of the C₁-C₂₀ alkyl is each optionally replaced independently with —CF₃, —CF₂H, —CH₂F, or a heteroatomic moiety selected from the group consisting of —NH(C₁-C₆ alkyl), —N(C₁-C₆ alkyl)₂, —O(C₁-C₆ alkyl), and —S(C₁-C₆ alkyl), and wherein any two heteroatomic moieties are separated from one another by at least two carbon atoms;
   L is a C₂-C₅₀ alkyl, C₂-C₅₀ alkenyl, C₂-C₅₀ alkynyl, C₃-C₈ cycloalkyl, aryl, heteroaryl, C₄-C₅₀ alkyl-cycloalkyl, C₇-C₅₀ alkyl-aryl, or C₆-C₅₀ alkyl-heteroaryl, wherein any one or more —CH₂— groups of L is each optionally replaced independently with —C(═O)—, —CF₂—, or a heteroatomic moiety selected from the group consisting of —O—, —S—, —NH—, and —N(C₁-C₆ alkyl)-, wherein any one or more —CH₃ groups of L is each optionally replaced independently with —CF₃, —CF₂H, —CH₂F, or a heteroatomic moiety selected from the group consisting of —NH(C₁-C₆ alkyl), —N(C₁-C₆alkyl)₂, —O(C₁-C₆ alkyl), and —S(C₁-C₆ alkyl), and wherein any two heteroatomic moieties are separated from one another by at least two carbon atoms;
   Z is a C₁-C₂₀ alkyl, wherein any one or more —CH₂— groups of the C₁-C₂₀ alkyl is each optionally replaced independently with —C(═O)—, —CF₂—, or a heteroatomic moiety selected from the group consisting of —O—, —S—, —NH—, and —N(C₁-C₆ alkyl)-, wherein any one or more —CH₃ groups of the C₁-C₂₀ alkyl is each optionally replaced independently with —CF₃, —CF₂H, —CH₂F, or a heteroatomic moiety selected from the group consisting of —NH(C₁-C₆ alkyl), —N(C₁-C₆ alkyl)₂, —O(C₁-C₆ alkyl), and —S(C₁-C₆ alkyl), and wherein any two heteroatomic moieties are separated from one another by at least two carbon atoms; and
   B is an oligonucleotide, a polypeptide, a protein, a small molecule, a lipid, a carbohydrate, a polyethylene glycol (PEG) molecule, or a biopolymer,
wherein Y is covalently attached to the 3′-end of A through a first linkage.

Embodiment 43. The oligonucleotide bioconjugate of Embodiment 42, wherein the first linkage is selected from the group consisting of a phosphate linkage, a phosphorothioate linkage, a phosphoramidate linkage, an amine linkage, an amide linkage, a triazole linkage, an ether linkage, and a thioether linkage.

Embodiment 44. The oligonucleotide bioconjugate of Embodiment 42 or Embodiment 43, wherein the first linkage is a phosphate linkage.

Embodiment 45. The oligonucleotide bioconjugate of Embodiment 44, wherein the phosphate linkage between Y and A comprises a phosphate linked to the 3′-oxygen atom of a nucleotide located at the 3′-end of A.

Embodiment 46. The oligonucleotide bioconjugate of any one of Embodiments 42 to 45, wherein A comprises a poly-adenosine monophosphate region or a poly-thymidine monophosphate region.

Embodiment 47. The oligonucleotide bioconjugate of any one of Embodiments 42 to 46, wherein the 5′-end of A is ligated to an mRNA molecule.

Embodiment 48. The oligonucleotide bioconjugate of any one of Embodiments 42 to 45, wherein A is an mRNA molecule.

Embodiment 49. The oligonucleotide bioconjugate of any one of Embodiments 42 to 48, wherein B is an oligonucleotide having a 3′-end and a 5′-end.

Embodiment 50. The oligonucleotide bioconjugate of Embodiment 49, wherein Z is covalently attached to the 3′-end of B through an second linkage selected from the group consisting of a phosphate linkage, a phosphorothioate linkage, a phosphoramidate linkage, an amine linkage, an amide linkage, a triazole linkage, an ether linkage, and a thioether linkage

Embodiment 51. The oligonucleotide bioconjugate of Embodiment 49 or Embodiment 50, wherein the second linkage is a phosphate linkage.

Embodiment 52. The oligonucleotide bioconjugate of Embodiment 51, wherein the phosphate linkage between Z and B comprises a phosphate linked to the 3′-oxygen atom of a nucleotide located at the 3′-end of B.

Embodiment 53. The oligonucleotide bioconjugate of any one of Embodiments 49 to 52, wherein B comprises a poly-adenosine monophosphate region or a poly-thymidine monophosphate region

Embodiment 54. The oligonucleotide bioconjugate of any one of Embodiments 49 to 53, wherein the 5′-end of B is ligated to an mRNA molecule.

Embodiment 55. The oligonucleotide bioconjugate of any one of Embodiments 49 to 52, wherein B is an mRNA molecule.

Embodiment 56. The oligonucleotide bioconjugate of any one of Embodiments 42 to 48, wherein B is a DNA molecule.

Embodiment 57. The oligonucleotide bioconjugate of any one of Embodiments 42 to 48, wherein B is a polypeptide.

Embodiment 58. The oligonucleotide bioconjugate of any one of Embodiments 42 to 48, wherein B is a protein.

Embodiment 59. The oligonucleotide bioconjugate of Embodiment 58, wherein B is an antibody.

Embodiment 60. The oligonucleotide bioconjugate of any one of Embodiments 42 to 48, wherein B is a small molecule.

Embodiment 61. The oligonucleotide bioconjugate of any one of Embodiments 42 to 48, wherein B is a carbohydrate.

Embodiment 62. The oligonucleotide bioconjugate of any one of Embodiments 42 to 48, wherein B is a lipid.

Embodiment 63. The oligonucleotide bioconjugate of any one of Embodiments 42 to 48, wherein B is a PEG molecule

Embodiment 64. The oligonucleotide bioconjugate of Embodiment 63, wherein the PEG molecule is selected from the group consisting of PEG₄₀₀, PEG₁₅₀₀, PEG₃₃₅₀, PEG₄₀₀₀, PEG₆₀₀₀, PEG₈₀₀₀, PEG₁₀₀₀₀, PEG₁₅₀₀₀, and PEG₂₀₀₀₀.

Embodiment 65. The oligonucleotide bioconjugate of any one of Embodiments 42 to 48, wherein B is a biopolymer.

Embodiment 66. The oligonucleotide bioconjugate of Embodiment 65, wherein the biopolymer is selected from the group consisting of proline/alanine/serine (PAS), XTEN, polysarcosine (pSar), polysaccharide, polyvinylpyrrolidone (PVP), polyglutamic acid (PGA), poly(hydroxyethyl-1-asparagine) (PHEA), poly(hydroxyethyl-1-glutamine) (PHEG), and poly(thioglycidyl glycerol) (PTTG).

Embodiment 67. The oligonucleotide bioconjugate of any one of Embodiments 42 to 66, wherein Y is —CH₂—.

Embodiment 68. The oligonucleotide bioconjugate of any one of Embodiments 42 to 66, wherein Y is —CH₂CH₂—.

Embodiment 69. The oligonucleotide bioconjugate of any one of Embodiments 42 to 66, wherein Y is *—(CH₂)₂O(CH₂)₂—, wherein * indicates the attachment point to A.

Embodiment 70. The oligonucleotide bioconjugate of any one of Embodiments 42 to 66, wherein Y is *—(CH₂)₂O(CH₂)₂O(CH₂)₂—, wherein * indicates the attachment point to A.

Embodiment 71. The oligonucleotide bioconjugate of any one of Embodiments 42 to 66, wherein Y is —CH₂CH(OCH₃)CH₂—.

Embodiment 72. The oligonucleotide bioconjugate of any one of Embodiments 42 to 66, wherein Y comprises a polyethylene glycol (PEG) moiety.

Embodiment 73. The oligonucleotide bioconjugate of any one of Embodiments 42 to 66, wherein Y comprises a polyamide moiety.

Embodiment 74. The oligonucleotide bioconjugate of any one of Embodiments 42 to 66, wherein Y comprises an acyl group.

Embodiment 75. The oligonucleotide bioconjugate of any one of Embodiments 42 to 74, wherein Z is —CH₂—.

Embodiment 76. The oligonucleotide bioconjugate of any one of Embodiments 42 to 74, wherein Z is —CH₂CH₂—.

Embodiment 77. The oligonucleotide bioconjugate of any one of Embodiments 42 to 74, wherein Z is —(CH₂)₂O(CH₂)₂—*, wherein * indicates the attachment point to B.

Embodiment 78. The oligonucleotide bioconjugate of any one of Embodiments 42 to 74, wherein Z is —(CH₂)₂O(CH₂)₂O(CH₂)₂—*, wherein * indicates the attachment point to B.

Embodiment 79. The oligonucleotide bioconjugate of any one of Embodiments 42 to 74, wherein Y is —CH₂CH(OCH₃)CH₂—.

Embodiment 80. The oligonucleotide bioconjugate of any one of Embodiments 42 to 74, wherein Z comprises a polyethylene glycol (PEG) moiety.

Embodiment 81. The oligonucleotide bioconjugate of any one of Embodiments 42 to 74, wherein Z comprises a polyamide moiety.

Embodiment 82. The oligonucleotide bioconjugate of any one of Embodiments 42 to 74, wherein Z comprises an acyl group.

Embodiment 83. The oligonucleotide bioconjugate of any one of Embodiments 42 to 82, wherein L is a C₂-C₅₀ alkyl.

Embodiment 84. The oligonucleotide bioconjugate of any one of Embodiments 42 to 82, wherein L comprises a polyethylene glycol (PEG) moiety.

Embodiment 85. The oligonucleotide bioconjugate of Embodiment 84, wherein L is a PEG₃ moiety.

Embodiment 86. The oligonucleotide bioconjugate of Embodiment 84, wherein L is a PEG₁₉ moiety.

Embodiment 87. The oligonucleotide bioconjugate of any one of Embodiments 42 to 82, wherein L comprises a polyamide moiety.

Embodiment 88. The oligonucleotide bioconjugate of any one of Embodiments 42 to 82, wherein L is a polypeptide.

Embodiment 89. The oligonucleotide bioconjugate of any one of Embodiments 42 to 82, wherein L comprises an acyl group.

Embodiment 90. The oligonucleotide bioconjugate of any one of Embodiments 42 to 82, wherein L is an aryl group.

Embodiment 91. The oligonucleotide bioconjugate of any one of Embodiments 42 to 82, wherein L comprises an aryl group.

Embodiment 92. An oligonucleotide bioconjugate of formula (III)

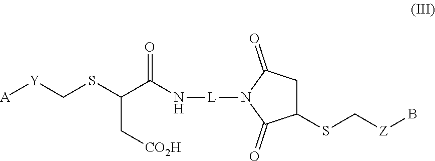

or a pharmaceutically acceptable salt thereof,
wherein
   A is an oligonucleotide having a 3′-end and a 5′-end;
   Y is a C₁-C₂₀ alkyl, wherein any one or more —CH₂— groups of the C₁-C₂₀ alkyl is each optionally replaced independently with —C(═O)—, —CF₂—, or a heteroatomic moiety selected from the group consisting of —O—, —S—, —NH—, and —N(C₁-C₆ alkyl)-, wherein any one or more —CH₃ groups of the C₁-C₂₀ alkyl is each optionally replaced independently with —CF₃, —CF₂H, —CH₂F, or a heteroatomic moiety selected from the group consisting of —NH(C₁-C₆ alkyl), —N(C₁-C₆ alkyl)₂, —O(C₁-C₆ alkyl), and —S(C₁-C₆ alkyl), and wherein any two heteroatomic moieties are separated from one another by at least two carbon atoms;
   L is a C₂-C₅₀ alkyl, C₂-C₅₀ alkenyl, C₂-C₅₀ alkynyl, C₃-C₈ cycloalkyl, aryl, heteroaryl, C₄-C₅₀ alkyl-cycloalkyl, C₇-C₅₀ alkyl-aryl, or C₆-C₅₀ alkyl-heteroaryl, wherein any one or more —CH₂— groups of L is each optionally replaced independently with —C(═O)—, —CF₂—, or a heteroatomic moiety selected from the group consisting of —O—, —S—, —NH—, and —N(C₁-C₆ alkyl)-, wherein any one or more —CH₃ groups of L is each optionally replaced independently with —CF₃, —CF₂H, —CH₂F, or a heteroatomic moiety selected from the group consisting of —NH(C₁-C₆ alkyl), —N(C₁-C₆alkyl)₂, —O(C₁-C₆ alkyl), and —S(C₁-C₆ alkyl), and wherein any two heteroatomic moieties are separated from one another by at least two carbon atoms;
   Z is a C₁-C₂₀ alkyl, wherein any one or more —CH₂— groups of the C₁-C₂₀ alkyl is each optionally replaced independently with —C(═O)—, —CF₂—, or a heteroatomic moiety selected from the group consisting of —O—, —S—, —NH—, and —N(C₁-C₆ alkyl)-, wherein any one or more —CH₃ groups of the C₁-C₂₀ alkyl is each optionally replaced independently with —CF₃, —CF₂H, —CH₂F, or a heteroatomic moiety selected from the group consisting of —NH(C₁-C₆ alkyl), —N(C₁-C₆ alkyl)₂, —O(C₁-C₆ alkyl), and —S(C₁-C₆ alkyl), and wherein any two heteroatomic moieties are separated from one another by at least two carbon atoms; and
   B is an oligonucleotide, a polypeptide, a protein, a small molecule, a lipid, a carbohydrate, a polyethylene glycol (PEG) molecule, or a biopolymer, wherein Y is covalently attached to the 3′-end of A through a first linkage.

Embodiment 93. The oligonucleotide bioconjugate of Embodiment 92, wherein the first linkage is selected from the group consisting of a phosphate linkage, a phosphorothioate linkage, a phosphoramidate linkage, an amine linkage, an amide linkage, a triazole linkage, an ether linkage, and a thioether linkage.

Embodiment 94. The oligonucleotide bioconjugate of Embodiment 92 or Embodiment 93, wherein the first linkage is a phosphate linkage.

Embodiment 95. The oligonucleotide bioconjugate of Embodiment 94, wherein the phosphate linkage between Y and A comprises a phosphate linked to the 3′-oxygen atom of a nucleotide located at the 3′-end of A.

Embodiment 96. The oligonucleotide bioconjugate of any one of Embodiments 92 to 95, wherein A is an mRNA molecule.

Embodiment 97. The oligonucleotide bioconjugate of any one of Embodiments 92 to 96, wherein B is an oligonucleotide having a 3′-end and a 5′-end.

Embodiment 98. The oligonucleotide bioconjugate of Embodiment 97, wherein Z is covalently attached to the 3′-end of B through a second linkage selected from the group consisting of a phosphate linkage, a phosphorothioate linkage, a phosphoramidate linkage, an amine linkage, an amide linkage, a triazole linkage, an ether linkage, and a thioether linkage.

Embodiment 99. The oligonucleotide bioconjugate of Embodiment 98, wherein the second linkage is a phosphate linkage.

Embodiment 100. The oligonucleotide bioconjugate of Embodiment 99, wherein the phosphate linkage between Z and B comprises a phosphate linked to the 3′-oxygen atom of a nucleotide located at the 3′-end of B.

Embodiment 101. The oligonucleotide bioconjugate of any one of Embodiments 92 to 100, wherein B is an mRNA molecule.

Embodiment 102. The oligonucleotide bioconjugate of any one of Embodiments 92 to 101, wherein L is a C₂-C₅₀ alkyl.

Embodiment 103. The oligonucleotide bioconjugate of any one of Embodiments 92 to 101, wherein L comprises a polyethylene glycol (PEG) moiety.

Embodiment 104. The oligonucleotide bioconjugate of Embodiment 103, wherein L is a PEG₃ moiety.

Embodiment 105. The oligonucleotide bioconjugate of Embodiment 103, wherein L is a PEG₁₉ moiety.

Embodiment 106. The oligonucleotide bioconjugate of any one of Embodiments 92 to 101, wherein L comprises a polyamide moiety.

Embodiment 107. The oligonucleotide bioconjugate of any one of Embodiments 92 to 101, wherein L is a polypeptide.

Embodiment 108. The oligonucleotide bioconjugate of any one of Embodiments 92 to 101, wherein L comprises an acyl group.

Embodiment 109. The oligonucleotide bioconjugate of any one of Embodiments 92 to 101, wherein L is an aryl group.

Embodiment 110. The oligonucleotide bioconjugate of any one of Embodiments 92 to 101, wherein L comprises an aryl group.

Embodiment 111. An oligonucleotide bioconjugate of formula (IV)

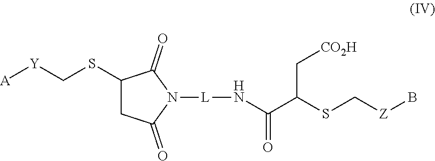

or a pharmaceutically acceptable salt thereof,
wherein
   A is an oligonucleotide having a 3′-end and a 5′-end;
   Y is a C₁-C₂₀ alkyl, wherein any one or more —CH₂— groups of the C₁-C₂₀ alkyl is each optionally replaced independently with —C(═O)—, —CF₂—, or a heteroatomic moiety selected from the group consisting of —O—, —S—, —NH—, and —N(C₁-C₆ alkyl)-, wherein any one or more —CH₃ groups of the C₁-C₂₀ alkyl is each optionally replaced independently with —CF₃, —CF₂H, —CH₂F, or a heteroatomic moiety selected from the group consisting of —NH(C₁-C₆ alkyl), —N(C₁-C₆ alkyl)₂, —O(C₁-C₆ alkyl), and —S(C₁-C₆ alkyl), and wherein any two heteroatomic moieties are separated from one another by at least two carbon atoms;
   L is a C₂-C₅₀ alkyl, C₂-C₅₀ alkenyl, C₂-C₅₀ alkynyl, C₃-C₈ cycloalkyl, aryl, heteroaryl, C₄-C₅₀ alkyl-cycloalkyl, C₇-C₅₀ alkyl-aryl, or C₆-C₅₀ alkyl-heteroaryl, wherein any one or more —CH₂— groups of L is each optionally replaced independently with —C(═O)—, —CF₂—, or a heteroatomic moiety selected from the group consisting of —O—, —S—, —NH—, and —N(C₁-C₆ alkyl)-, wherein any one or more —CH₃ groups of L is each optionally replaced independently with —CF₃, —CF₂H, —CH₂F, or a heteroatomic moiety selected from the group consisting of —NH(C₁-C₆ alkyl), —N(C₁-C₆ alkyl)₂, —O(C₁-C₆ alkyl), and —S(C₁-C₆ alkyl), and wherein any two heteroatomic moieties are separated from one another by at least two carbon atoms;
   Z is a C₁-C₂₀ alkyl, wherein any one or more —CH₂— groups of the C₁-C₂₀ alkyl is each optionally replaced independently with —C(═O)—, —CF₂—, or a heteroatomic moiety selected from the group consisting of —O—, —S—, —NH—, and —N(C₁-C₆ alkyl)-, wherein any one or more —CH₃ groups of the C₁-C₂₀ alkyl is each optionally replaced independently with —CF₃, —CF₂H, —CH₂F, or a heteroatomic moiety selected from the group consisting of —NH(C₁-C₆ alkyl), —N(C₁-C₆ alkyl)₂, —O(C₁-C₆ alkyl), and —S(C₁-C₆ alkyl), and wherein any two heteroatomic moieties are separated from one another by at least two carbon atoms; and
   B is an oligonucleotide, a polypeptide, a protein, a small molecule, a lipid, a carbohydrate, a polyethylene glycol (PEG) molecule, or a biopolymer,
wherein Y is covalently attached to the 3′-end of A through a first linkage.

Embodiment 112. The oligonucleotide bioconjugate of Embodiment 111, wherein the first linkage is selected from the group consisting of a phosphate linkage, a phosphorothioate linkage, a phosphoramidate linkage, an amine linkage, an amide linkage, a triazole linkage, an ether linkage, and a thioether linkage.

Embodiment 113. The oligonucleotide bioconjugate of Embodiment 111 or Embodiment 112, wherein the first linkage is a phosphate linkage.

Embodiment 114. The oligonucleotide bioconjugate of Embodiment 113, wherein the phosphate linkage between Y and A comprises a phosphate linked to the 3′-oxygen atom of a nucleotide located at the 3′-end of A.

Embodiment 115. The oligonucleotide bioconjugate of any one of Embodiments 111 to 114, wherein A is an mRNA molecule.

Embodiment 116. The oligonucleotide bioconjugate of any one of Embodiments 111 to 115, wherein B is an oligonucleotide having a 3′-end and a 5′-end.

Embodiment 117. The oligonucleotide bioconjugate of Embodiment 116, wherein Z is covalently attached to the 3′-end of B through a second linkage selected from the group consisting of a phosphate linkage, a phosphorothioate linkage, a phosphoramidate linkage, an amine linkage, an amide linkage, a triazole linkage, an ether linkage, and a thioether linkage.

Embodiment 118. The oligonucleotide bioconjugate of Embodiment 117, wherein the second linkage is a phosphate linkage.

Embodiment 119. The oligonucleotide bioconjugate of Embodiment 118, wherein the phosphate linkage between Z and B comprises a phosphate linked to the 3′-oxygen atom of a nucleotide located at the 3′-end of B.

Embodiment 120. The oligonucleotide bioconjugate of any one of Embodiments 111 to 119, wherein B is an mRNA molecule.

Embodiment 121. The oligonucleotide bioconjugate of any one of Embodiments 111 to 120, wherein L is a C₂-C₅₀ alkyl.

Embodiment 122. The oligonucleotide bioconjugate of any one of Embodiments 111 to 120, wherein L comprises a polyethylene glycol (PEG) moiety.

Embodiment 123. The oligonucleotide bioconjugate of Embodiment 122, wherein L is a PEG₃ moiety.

Embodiment 124. The oligonucleotide bioconjugate of Embodiment 122, wherein L is a PEG₁₉ moiety.

Embodiment 125. The oligonucleotide bioconjugate of any one of Embodiments 111 to 120, wherein L comprises a polyamide moiety.

Embodiment 126. The oligonucleotide bioconjugate of any one of Embodiments 111 to 120, wherein L is a polypeptide.

Embodiment 127. The oligonucleotide bioconjugate of any one of Embodiments 111 to 120, wherein L comprises an acyl group.

Embodiment 128. The oligonucleotide bioconjugate of any one of Embodiments 111 to 120, wherein L is an aryl group.

Embodiment 129. The oligonucleotide bioconjugate of any one of Embodiments 111 to 120, wherein L comprises an aryl group.

Embodiment 130. An oligonucleotide bioconjugate of formula (V)

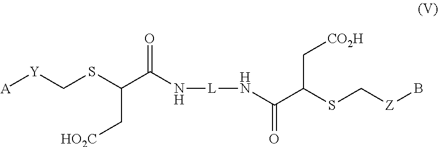

or a pharmaceutically acceptable salt thereof,
wherein
   A is an oligonucleotide having a 3′-end and a 5′-end;
   Y is a C₁-C₂₀ alkyl, wherein any one or more —CH₂— groups of the C₁-C₂₀ alkyl is each optionally replaced independently with —C(═O)—, —CF₂—, or a heteroatomic moiety selected from the group consisting of —O—, —S—, —NH—, and —N(C₁-C₆ alkyl)-, wherein any one or more —CH₃ groups of the C₁-C₂₀ alkyl is each optionally replaced independently with —CF₃, —CF₂H, —CH₂F, or a heteroatomic moiety selected from the group consisting of —NH(C₁-C₆ alkyl), —N(C₁-C₆ alkyl)₂, —O(C₁-C₆ alkyl), and —S(C₁-C₆ alkyl), and wherein any two heteroatomic moieties are separated from one another by at least two carbon atoms;
   L is a C₂-C₅₀ alkyl, C₂-C₅₀ alkenyl, C₂-C₅₀ alkynyl, C₃-C₈ cycloalkyl, aryl, heteroaryl, C₄-C₅₀ alkyl-cycloalkyl, C₇-C₅₀ alkyl-aryl, or C₆-C₅₀ alkyl-heteroaryl, wherein any one or more —CH₂— groups of the L is each optionally replaced independently with —C(═O)—, —CF₂—, or a heteroatomic moiety selected from the group consisting of —O—, —S—, —NH—, and —N(C₁-C₆ alkyl)-, wherein any one or more —CH₃ groups of L is each optionally replaced independently with —CF₃, —CF₂H, —CH₂F, or a heteroatomic moiety selected from the group consisting of —NH(C₁-C₆ alkyl), —N(C₁-C₆alkyl)₂, —O(C₁-C₆ alkyl), and —S(C₁-C₆ alkyl), and wherein any two heteroatomic moieties are separated from one another by at least two carbon atoms;
   Z is a C₁-C₂₀ alkyl, wherein any one or more —CH₂— groups of the C₁-C₂₀ alkyl is each optionally replaced independently with —C(═O)—, —CF₂—, or a heteroatomic moiety selected from the group consisting of —O—, —S—, —NH—, and —N(C₁-C₆ alkyl)-, wherein any one or more —CH₃ groups of the C₁-C₂₀ alkyl is each optionally replaced independently with —CF₃, —CF₂H, —CH₂F, or a heteroatomic moiety selected from the group consisting of —NH(C₁-C₆ alkyl), —N(C₁-C₆ alkyl)₂, —O(C₁-C₆ alkyl), and —S(C₁-C₆ alkyl), and wherein any two heteroatomic moieties are separated from one another by at least two carbon atoms; and
   B is an oligonucleotide, a polypeptide, a protein, a small molecule, a lipid, a carbohydrate, a polyethylene glycol (PEG) molecule, or a biopolymer, wherein Y is covalently attached to the 3′-end of A through a first linkage.

Embodiment 131. The oligonucleotide bioconjugate of Embodiment 130, wherein the first linkage is selected from the group consisting of a phosphate linkage, a phosphorothioate linkage, a phosphoramidate linkage, an amine linkage, an amide linkage, a triazole linkage, an ether linkage, and a thioether linkage.

Embodiment 132. The oligonucleotide bioconjugate of Embodiment 130 or Embodiment 131, wherein the first linkage is a phosphate linkage.

Embodiment 133. The oligonucleotide bioconjugate of Embodiment 132, wherein the phosphate linkage between Y and A comprises a phosphate linked to the 3′-oxygen atom of a nucleotide located at the 3′-end of A.

Embodiment 134. The oligonucleotide bioconjugate of any one of Embodiments 130 to 133, wherein A is an mRNA molecule.

Embodiment 135. The oligonucleotide bioconjugate of any one of Embodiments 130 to 134, wherein B is an oligonucleotide having a 3′-end and a 5′-end.

Embodiment 136. The oligonucleotide bioconjugate of Embodiment 135, wherein Z is covalently attached to the 3′-end of B through a second linkage selected from the group consisting of a phosphate linkage, a phosphorothioate linkage, a phosphoramidate linkage, an amine linkage, an amide linkage, a triazole linkage, an ether linkage, and a thioether linkage.

Embodiment 137. The oligonucleotide bioconjugate of Embodiment 136, wherein the second linkage is a phosphate linkage.

Embodiment 138. The oligonucleotide bioconjugate of Embodiment 137, wherein the phosphate linkage between Z and B comprises a phosphate linked to the 3′-oxygen atom of a nucleotide located at the 3′-end of B.

Embodiment 139. The oligonucleotide bioconjugate of any one of Embodiments 130 to 138, wherein B is an mRNA molecule.

Embodiment 140. The oligonucleotide bioconjugate of any one of Embodiments 130 to 139, wherein L is a C₂-C₅₀ alkyl.

Embodiment 141. The oligonucleotide bioconjugate of any one of Embodiments 130 to 139, wherein L comprises a polyethylene glycol (PEG) moiety.

Embodiment 142. The oligonucleotide bioconjugate of Embodiment 141, wherein L is a PEG₃ moiety,

Embodiment 143. The oligonucleotide bioconjugate of Embodiment 141, wherein L is a PEG₁₉ moiety.

Embodiment 144. The oligonucleotide bioconjugate of any one of Embodiments 130 to 139, wherein L comprises a polyamide moiety.

Embodiment 145. The oligonucleotide bioconjugate of any one of Embodiments 130 to 139, wherein L is a polypeptide.

Embodiment 146. The oligonucleotide bioconjugate of any one of Embodiments 130 to 139, wherein L comprises an acyl group.

Embodiment 147. The oligonucleotide bioconjugate of any one of Embodiments 130 to 139, wherein L is an aryl group.

Embodiment 148. The oligonucleotide bioconjugate of any one of Embodiments 130 to 139, wherein L comprises an aryl group.

Embodiment 149. A pharmaceutical formulation comprising the oligonucleotide bioconjugate of any one of Embodiments 1 to 148 and a pharmaceutically acceptable carrier, excipient, or diluent.

Embodiment 150. The pharmaceutical formulation of Embodiment 149, wherein the pharmaceutically acceptable carrier, excipient, or diluent comprises a lipid-based carrier, a polymer-based carrier, or a nanocarrier

Embodiment 151. The pharmaceutical formulation of Embodiment 150, wherein the lipid-based carrier is a liposome.

Embodiment 152. The pharmaceutical formulation of Embodiment 150, wherein the nanocarrier is a polypeptide nanoparticle, a dendrimer, a lipid nanoparticle, or a self-assembled protein.

Embodiment 153. The pharmaceutical formulation of Embodiment 152, wherein the self-assembled protein is a lentivirus or an adenovirus.

Embodiment 154. The pharmaceutical formulation of any one of Embodiments 149 to 153, for use in treating, preventing, slowing the progression, or reducing the severity of a disease, disorder, or condition in a patient in need thereof.

Embodiment 155. The pharmaceutical formulation of Embodiment 154, wherein the disease, disorder, or condition is selected from the group consisting of obesity and cancer.

Embodiment 156. The pharmaceutical formulation of any one of Embodiments 149 to 153, for use in enzyme replacement therapy (ERT) in a patient in need thereof, wherein the ERT treats one or more lysosomal storage diseases (LSDs).

Embodiment 157. The pharmaceutical formulation of any one of Embodiments 149 to 153, for use as a vaccine therapy in a patient in need thereof

Embodiment 158. The pharmaceutical formulation of Embodiment 157, wherein the vaccine therapy is for vaccination against an infectious disease.

Embodiment 159. The pharmaceutical formulation of any one of Embodiments 149 to 158, wherein the patient in need thereof is a human.

Embodiment 160. Use of the oligonucleotide bioconjugate of any one of Embodiments 1 to 148 for the manufacture of a medicament for treating, preventing, slowing the progression, or reducing the severity of a disease, disorder, or condition in a patient in need thereof.

Embodiment 161. The use of Embodiment 160, wherein the disease, disorder, or condition is selected from the group consisting of obesity and cancer.

Embodiment 162. Use of the oligonucleotide bioconjugate of any one of Embodiments 1 to 148 for the manufacture of a medicament for enzyme replacement therapy in a patient in need thereof.

Embodiment 163. Use of the oligonucleotide bioconjugate of any one of Embodiments 1 to 148 for the manufacture of a medicament for vaccination of a patient in need thereof.

Embodiment 164. The use of any one of Embodiments 160 to 163, wherein the patient in need thereof is a human.

Embodiment 165. A method of making an oligonucleotide bioconjugate of formula (I)

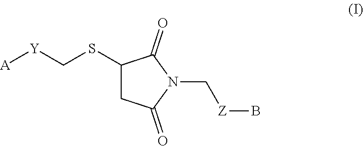

or a pharmaceutically acceptable salt thereof,
wherein
   A is an oligonucleotide having a 3′-end and a 5′-end;
   Y is a C₁-C₂₀ alkyl, wherein any one or more —CH₂— groups of the C₁-C₂₀ alkyl is each optionally replaced independently with —C(═O)—, —CF₂—, or a heteroatomic moiety selected from the group consisting of —O—, —S—, —NH—, and —N(C₁-C₆ alkyl)-, wherein any one or more —CH₃ groups of the C₁-C₂₀ alkyl is each optionally replaced independently with —CF₃, —CF₂H, —CH₂F, or a heteroatomic moiety selected from the group consisting of —NH(C₁-C₆ alkyl), —N(C₁-C₆ alkyl)₂, —O(C₁-C₆ alkyl), and —S(C₁-C₆ alkyl), and wherein any two heteroatomic moieties are separated from one another by at least two carbon atoms;
   Z is a C₁-C₂₀ alkyl, wherein any one or more —CH₂— groups of the C₁-C₂₀ alkyl is each optionally replaced independently with —C(═O)—, —CF₂—, or a heteroatomic moiety selected from the group consisting of —O—, —S—, —NH—, and —N(C₁-C₆ alkyl)-, wherein any one or more —CH₃ groups of the C₁-C₂₀ alkyl is each optionally replaced independently with —CF₃, —CF₂H, —CH₂F, or a heteroatomic moiety selected from the group consisting of —NH(C₁-C₆ alkyl), —N(C₁-C₆ alkyl)₂, —O(C₁-C₆ alkyl), and —S(C₁-C₆ alkyl), and wherein any two heteroatomic moieties are separated from one another by at least two carbon atoms; and
   B is an oligonucleotide, a polypeptide, a protein, a small molecule, a carbohydrate, a lipid, a polyethylene glycol (PEG) molecule, or a biopolymer,
wherein Y is covalently attached to the 3′-end of A through a first linkage, the method comprising a step of reacting the thiol of formula (VI) with the maleimide of formula (VII) to form the oligonucleotide bioconjugate of formula (I)

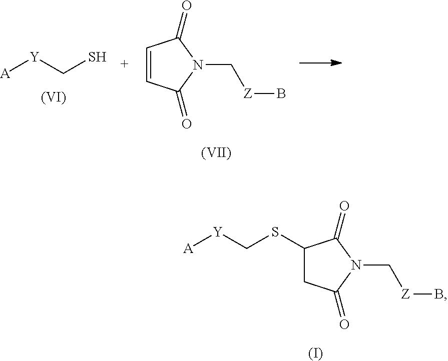

wherein A and Y of formula (VI) are defined as above for formula (I), and wherein Z and B of formula (VII) are defined as above for formula (I).

Embodiment 166. The method of Embodiment 165, wherein the first linkage is selected from the group consisting of a phosphate linkage, a phosphorothioate linkage, a phosphoramidate linkage, an amine linkage, an amide linkage, a triazole linkage, an ether linkage, and a thioether linkage.

Embodiment 167. The method of Embodiment 165 or Embodiment 166, wherein the first linkage is a phosphate linkage.

Embodiment 168. The method of Embodiment 167, wherein the phosphate linkage between Y and A comprises a phosphate linked to the 3′-oxygen atom of a nucleotide located at the 3′-end of A.

Embodiment 169. The method of any one of Embodiments 165 to 168, wherein B is an oligonucleotide having a 3′-end and a 5′-end.

Embodiment 170. The method of Embodiment 169, wherein Z is covalently attached to the 3′-end of B through a second linkage selected from the group consisting of a phosphate linkage, a phosphorothioate linkage, a phosphoramidate linkage, an amine linkage, an amide linkage, a triazole linkage, an ether linkage, and a thioether linkage.

Embodiment 171. The method of Embodiment 170, wherein the second linkage is a phosphate linkage.

Embodiment 172. The method of Embodiment 171, wherein the phosphate linkage between Z and B comprises a phosphate linked to the 3′-oxygen atom of a nucleotide located at the 3′-end of B.

Embodiment 173. A method of making an oligonucleotide bioconjugate of formula (II)

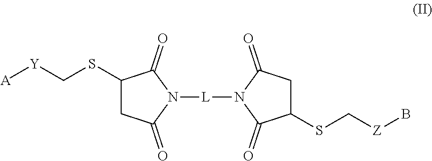

or a pharmaceutically acceptable salt thereof,
wherein
   A is an oligonucleotide having a 3′-end and a 5′-end;
   Y is a C₁-C₂₀ alkyl, wherein any one or more —CH₂— groups of the C₁-C₂₀ alkyl is each optionally replaced independently with —C(═O)—, —CF₂—, or a heteroatomic moiety selected from the group consisting of —O—, —S—, —NH—, and —N(C₁—C alkyl)-, wherein any one or more —CH₃ groups of the C₁-C₂₀ alkyl is each optionally replaced independently with —CF₃, —CF₂H, —CH₂F, or a heteroatomic moiety selected from the group consisting of —NH(C₁-C₆ alkyl), —N(C₁-C₆ alkyl)₂, —O(C₁-C₆ alkyl), and —S(C₁-C₆ alkyl), and wherein any two heteroatomic moieties are separated from one another by at least two carbon atoms;
   L is a C₂-C₅₀ alkyl, C₂-C₅₀ alkenyl, C₂-C₅₀ alkynyl, C₃-C₈ cycloalkyl, aryl, heteroaryl, C₄-C₅₀ alkyl-cycloalkyl, C₇-C₅₀ alkyl-aryl, or C₆-C₅₀ alkyl-heteroaryl, wherein any one or more —CH₂— groups of L is each optionally replaced independently with —C(═O)—, —CF₂—, or a heteroatomic moiety selected from the group consisting of —O—, —S—, —NH—, and —N(C₁-C₆ alkyl)-, wherein any one or more —CH₃ groups of L is each optionally replaced independently with —CF₃, —CF₂H, —CH₂F, or a heteroatomic moiety selected from the group consisting of —NH(C₁-C₆ alkyl), —N(C₁-C₆alkyl)₂, —O(C₁-C₆ alkyl), and —S(C₁-C₆ alkyl), and wherein any two heteroatomic moieties are separated from one another by at least two carbon atoms;
   Z is a C₁-C₂₀ alkyl, wherein any one or more —CH₂— groups of the C₁-C₂₀ alkyl is each optionally replaced independently with —C(═O)—, —CF₂—, or a heteroatomic moiety selected from the group consisting of —O—, —S—, —NH—, and —N(C₁-C₆ alkyl)-, wherein any one or more —CH₃ groups of the C₁-C₂₀ alkyl is each optionally replaced independently with —CF₃, —CF₂H, —CH₂F, or a heteroatomic moiety selected from the group consisting of —NH(C₁-C₆ alkyl), —N(C₁-C₆ alkyl)₂, —O(C₁-C₆ alkyl), and —S(C₁-C₆ alkyl), and wherein any two heteroatomic moieties are separated from one another by at least two carbon atoms; and
   B is an oligonucleotide, a polypeptide, a protein, a small molecule, a lipid, a carbohydrate, a polyethylene glycol (PEG) molecule, or a biopolymer,
   wherein Y is covalently attached to the 3′-end of A through a first linkage,
the method comprising the steps of
(i) reacting the thiol of formula (VI) with one of the two maleimides of formula (VIII) to form a compound of formula (IX)

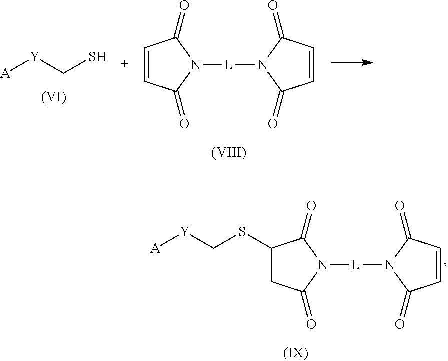

wherein A and Y of formula (VI) are defined as above for formula (II), and wherein L of formula (VIII) is defined as above for formula (II), and
(ii) reacting the maleimide of formula (IX) with the thiol of formula (X) to form the oligonucleotide bioconjugate of formula (II)

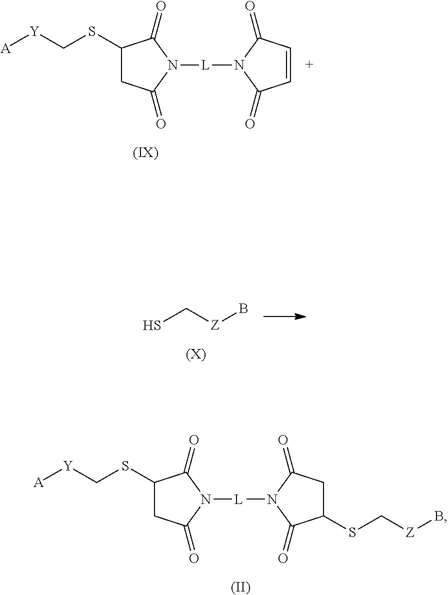

wherein Z and B of formula (X) are as defined above for formula (II).

Embodiment 174. The method of Embodiment 173, wherein the first linkage is selected from the group consisting of a phosphate linkage, a phosphorothioate linkage, a phosphoramidate linkage, an amine linkage, an amide linkage, a triazole linkage, an ether linkage, and a thioether linkage.

Embodiment 175. The method of Embodiment 173 or Embodiment 174, wherein the first linkage is a phosphate linkage.

Embodiment 176. The method of Embodiment 175, wherein the phosphate linkage between Y and A comprises a phosphate linked to the 3′-oxygen atom of a nucleotide located at the 3′-end of A.

Embodiment 177. The method of any one of Embodiments 173 to 176, wherein B is an oligonucleotide having a 3′-end and a 5′-end.

Embodiment 178. The method of Embodiment 177, wherein Z is covalently attached to the 3′-end of B through a second linkage selected from the group consisting of a phosphate linkage, a phosphorothioate linkage, a phosphoramidate linkage, an amine linkage, an amide linkage, a triazole linkage, an ether linkage, and a thioether linkage.

Embodiment 179. The method of Embodiment 178, wherein the second linkage is a phosphate linkage.

Embodiment 180. The method of Embodiment 179, wherein the phosphate linkage between Z and B comprises a phosphate linked to the 3′-oxygen atom of a nucleotide located at the 3′-end of B.

Embodiment 181. A method of making an oligonucleotide bioconjugate of formula (III)

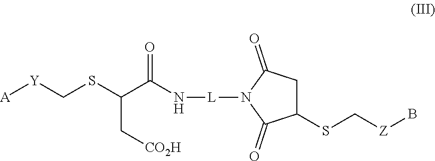

or a pharmaceutically acceptable salt thereof,
wherein
   A is an oligonucleotide having a 3′-end and a 5′-end;
   Y is a C₁-C₂₀ alkyl, wherein any one or more —CH₂— groups of the C₁-C₂₀ alkyl is each optionally replaced independently with —C(═O)—, —CF₂—, or a heteroatomic moiety selected from the group consisting of —O—, —S—, —NH—, and —N(C₁-C₆ alkyl)-, wherein any one or more —CH₃ groups of the C₁-C₂₀ alkyl is each optionally replaced independently with —CF₃, —CF₂H, —CH₂F, or a heteroatomic moiety selected from the group consisting of —NH(C₁-C₆ alkyl), —N(C₁-C₆ alkyl)₂, —O(C₁-C₆ alkyl), and —S(C₁-C₆ alkyl), and wherein any two heteroatomic moieties are separated from one another by at least two carbon atoms;
   L is a C₂-C₅₀ alkyl, C₂-C₅₀ alkenyl, C₂-C₅₀ alkynyl, C₃-C₈ cycloalkyl, aryl, heteroaryl, C₄-C₅₀ alkyl-cycloalkyl, C₇-C₅₀ alkyl-aryl, or C₆-C₅₀ alkyl-heteroaryl, wherein any one or more —CH₂— groups of L is each optionally replaced independently with —C(═O)—, —CF₂—, or a heteroatomic moiety selected from the group consisting of —O—, —S—, —NH—, and —N(C₁-C₆ alkyl)-, wherein any one or more —CH₃ groups of L is each optionally replaced independently with —CF₃, —CF₂H, —CH₂F, or a heteroatomic moiety selected from the group consisting of —NH(C₁-C₆ alkyl), —N(C₁—C₆ alkyl)₂, —O(C₁-C₆ alkyl), and —S(C₁-C₆ alkyl), and wherein any two heteroatomic moieties are separated from one another by at least two carbon atoms;
   Z is a C₁-C₂₀ alkyl, wherein any one or more —CH₂— groups of the C₁-C₂₀ alkyl is each optionally replaced independently with —C(═O)—, —CF₂—, or a heteroatomic moiety selected from the group consisting of —O—, —S—, —NH—, and —N(C₁-C₆ alkyl)-, wherein any one or more —CH₃ groups of the C₁-C₂₀ alkyl is each optionally replaced independently with —CF₃, —CF₂H, —CH₂F, or a heteroatomic moiety selected from the group consisting of —NH(C₁-C₆ alkyl), —N(C₁-C₆ alkyl)₂, —O(C₁-C₆ alkyl), and —S(C₁-C₆ alkyl), and wherein any two heteroatomic moieties are separated from one another by at least two carbon atoms; and
   B is an oligonucleotide, a polypeptide, a protein, a small molecule, a lipid, a carbohydrate, a polyethylene glycol (PEG) molecule, or a biopolymer,
wherein Y is covalently attached to the 3′-end of A through a first linkage,
the method comprising the steps of
(i) reacting the thiol of formula (VI) with one of the two maleimides of formula (VIII) to form a compound of formula (IX)

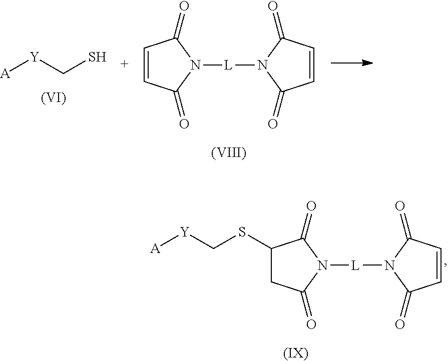

wherein A and Y of formula (VI) are defined as above for formula (III), and wherein L of formula (VIII) is defined as above for formula (III),
(ii) reacting the maleimide of formula (IX) with the thiol of formula (X) to form a compound of formula (II)

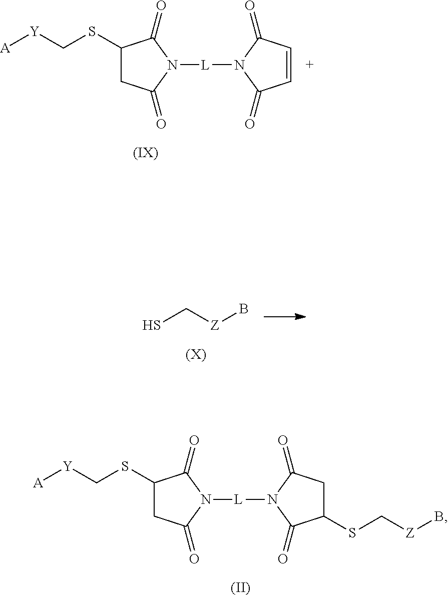

wherein Z and B of formula (X) are as defined above for formula (III), and
(iii) hydrolyzing one of the succinimides of formula (II) to form the oligonucleotide bioconjugate of formula (III)

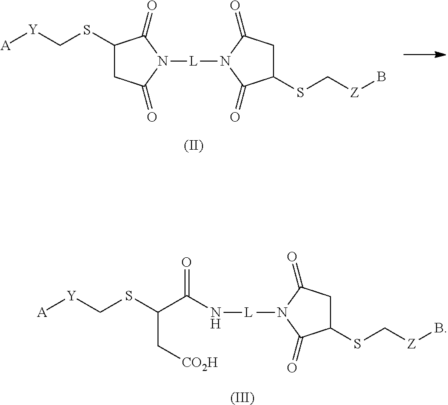

Embodiment 182. The method of Embodiment 181, wherein the first linkage is selected from the group consisting of a phosphate linkage, a phosphorothioate linkage, a phosphoramidate linkage, an amine linkage, an amide linkage, a triazole linkage, an ether linkage, and a thioether linkage.

Embodiment 183. The method of Embodiment 181 or Embodiment 182, wherein the first linkage is a phosphate linkage.

Embodiment 184. The method of Embodiment 183, wherein the phosphate linkage between Y and A comprises a phosphate linked to the 3′-oxygen atom of a nucleotide located at the 3′-end of A.

Embodiment 185. The method of any one of Embodiments 181 to 184, wherein B is an oligonucleotide having a 3′-end and a 5′-end.

Embodiment 186. The method of Embodiment 185, wherein Z is covalently attached to the 3′-end of B through a second linkage selected from the group consisting of a phosphate linkage, a phosphorothioate linkage, a phosphoramidate linkage, an amine linkage, an amide linkage, a triazole linkage, an ether linkage, and a thioether linkage.

Embodiment 187. The method of Embodiment 186, wherein the second linkage is a phosphate linkage.

Embodiment 188. The method of Embodiment 187, wherein the phosphate linkage between Z and B comprises a phosphate linked to the 3′-oxygen atom of a nucleotide located at the 3′-end of B.

Embodiment 189. A method of making an oligonucleotide bioconjugate of formula (IV)

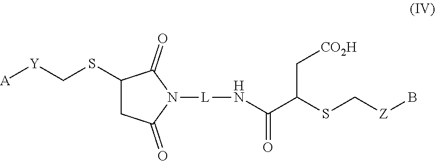

or a pharmaceutically acceptable salt thereof,
wherein
   A is an oligonucleotide having a 3′-end and a 5′-end;
   Y is a C₁-C₂₀ alkyl, wherein any one or more —CH₂— groups of the C₁-C₂₀ alkyl is each optionally replaced independently with —C(═O)—, —CF₂—, or a heteroatomic moiety selected from the group consisting of —O—, —S—, —NH—, and —N(C₁-C₆ alkyl)-, wherein any one or more —CH₃ groups of the C₁-C₂₀ alkyl is each optionally replaced independently with —CF₃, —CF₂H, —CH₂F, or a heteroatomic moiety selected from the group consisting of —NH(C₁-C₆ alkyl), —N(C₁-C₆ alkyl)₂, —O(C₁-C₆ alkyl), and —S(C₁-C₆ alkyl), and wherein any two heteroatomic moieties are separated from one another by at least two carbon atoms;
   L is a C₂-C₅₀ alkyl, C₂-C₅₀ alkenyl, C₂-C₅₀ alkynyl, C₃-C₈ cycloalkyl, aryl, heteroaryl, C₄-C₅₀ alkyl-cycloalkyl, C₇-C₅₀ alkyl-aryl, or C₆-C₅₀ alkyl-heteroaryl, wherein any one or more —CH₂— groups of L is each optionally replaced independently with —C(═O)—, —CF₂—, or a heteroatomic moiety selected from the group consisting of —O—, —S—, —NH—, and —N(C₁-C₆ alkyl)-, wherein any one or more —CH₃ groups of L is each optionally replaced independently with —CF₃, —CF₂H, —CH₂F, or a heteroatomic moiety selected from the group consisting of —NH(C₁-C₆ alkyl), —N(C₁-C₆ alkyl)₂, —O(C₁-C₆ alkyl), and —S(C₁-C₆ alkyl), and wherein any two heteroatomic moieties are separated from one another by at least two carbon atoms;
   Z is a C₁-C₂₀ alkyl, wherein any one or more —CH₂— groups of the C₁-C₂₀ alkyl is each optionally replaced independently with —C(═O)—, —CF₂—, or a heteroatomic moiety selected from the group consisting of —O—, —S—, —NH—, and —N(C₁-C₆ alkyl)-, wherein any one or more —CH₃ groups of the C₁-C₂₀ alkyl is each optionally replaced independently with —CF₃, —CF₂H, —CH₂F, or a heteroatomic moiety selected from the group consisting of —NH(C₁-C₆ alkyl), —N(C₁-C₆ alkyl)₂, —O(C₁-C₆ alkyl), and —S(C₁-C₆ alkyl), and wherein any two heteroatomic moieties are separated from one another by at least two carbon atoms; and
   B is an oligonucleotide, a polypeptide, a protein, a small molecule, a lipid, a carbohydrate, a polyethylene glycol (PEG) molecule, or a biopolymer,
wherein Y is covalently attached to the 3′-end of A through a first linkage,
the method comprising the steps of
(i) reacting the thiol of formula (VI) with the maleimide of formula (VIII) to form a compound of formula (IX)

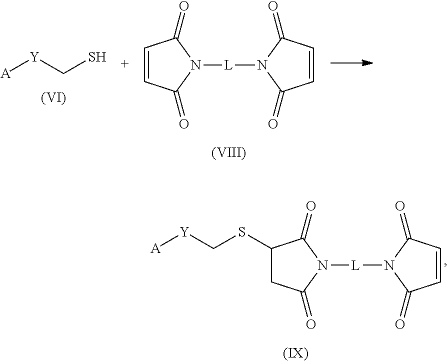

wherein A and Y of formula (VI) are defined as above for formula (IV), and wherein L of formula (VIII) is defined as above for formula (IV),
(ii) reacting the maleimide of formula (IX) with the thiol of formula (X) to form a compound of formula (II)

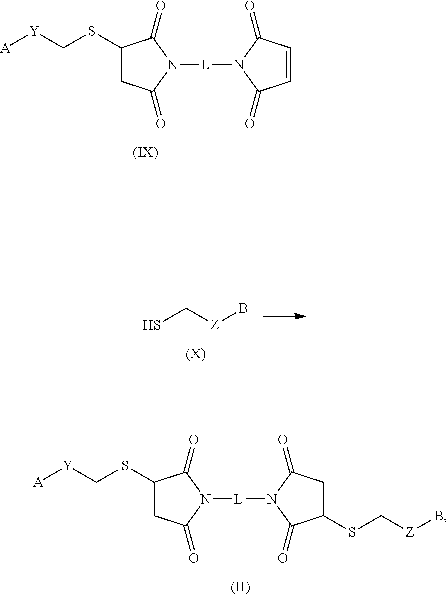

wherein Z and B of formula (X) are as defined above for formula (IV), and
(iii) hydrolyzing one of the succinimides of formula (II) to form the oligonucleotide bioconjugate of formula (IV)

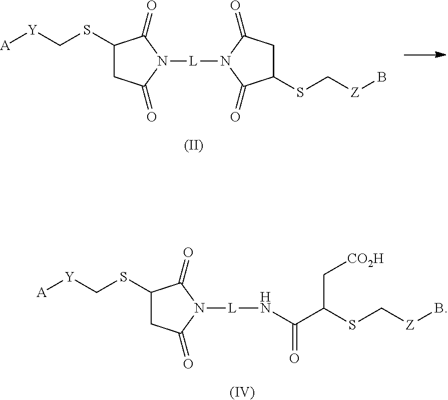

Embodiment 190. The method of Embodiment 189, wherein the first linkage is selected from the group consisting of a phosphate linkage, a phosphorothioate linkage, a phosphoramidate linkage, an amine linkage, an amide linkage, a triazole linkage, an ether linkage, and a thioether linkage.

Embodiment 191. The method of Embodiment 190, wherein the first linkage is a phosphate linkage.

Embodiment 192. The method of Embodiment 190 or Embodiment 191, wherein the phosphate linkage between Y and A comprises a phosphate linked to the 3′-oxygen atom of a nucleotide located at the 3′-end of A.

Embodiment 193. The method of any one of Embodiments 189 to 192, wherein B is an oligonucleotide having a 3′-end and a 5′-end.

Embodiment 194. The method of Embodiment 193, wherein Z is covalently attached to the 3′-end of B through a second linkage selected from the group consisting of a phosphate linkage, a phosphorothioate linkage, a phosphoramidate linkage, an amine linkage, an amide linkage, a triazole linkage, an ether linkage, and a thioether linkage.

Embodiment 195. The method of Embodiment 194, wherein the second linkage is a phosphate linkage.

Embodiment 196. The method of Embodiment 195, wherein the phosphate linkage between Z and B comprises a phosphate linked to the 3′-oxygen atom of a nucleotide located at the 3′-end of B.

Embodiment 197. A method of making an oligonucleotide bioconjugate of formula

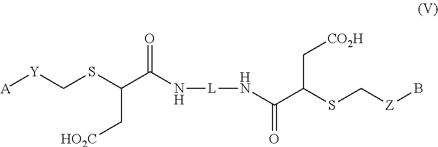

or a pharmaceutically acceptable salt thereof,
wherein
   A is an oligonucleotide having a 3′-end and a 5′-end;
   Y is a C₁-C₂₀ alkyl, wherein any one or more —CH₂— groups of the C₁-C₂₀ alkyl is each optionally replaced independently with —C(═O)—, —CF₂—, or a heteroatomic moiety selected from the group consisting of —O—, —S—, —NH—, and —N(C₁-C₆ alkyl)-, wherein any one or more —CH₃ groups of the C₁-C₂₀ alkyl is each optionally replaced independently with —CF₃, —CF₂H, —CH₂F, or a heteroatomic moiety selected from the group consisting of —NH(C₁-C₆ alkyl), —N(C₁-C₆ alkyl)₂, —O(C₁-C₆ alkyl), and —S(C₁-C₆ alkyl), and wherein any two heteroatomic moieties are separated from one another by at least two carbon atoms;
   L is a C₂-C₅₀ alkyl, C₂-C₅₀ alkenyl, C₂-C₅₀ alkynyl, C₃-C₈ cycloalkyl, aryl, heteroaryl, C₄-C₅₀ alkyl-cycloalkyl, C₇-C₅₀ alkyl-aryl, or C₆-C₅₀ alkyl-heteroaryl, wherein any one or more —CH₂— groups of L is each optionally replaced independently with —C(═O)—, —CF₂—, or a heteroatomic moiety selected from the group consisting of —O—, —S—, —NH—, and —N(C₁-C₆ alkyl)-, wherein any one or more —CH₃ groups of L is each optionally replaced independently with —CF₃, —CF₂H, —CH₂F, or a heteroatomic moiety selected from the group consisting of —NH(C₁-C₆ alkyl), —N(C₁-C₆ alkyl)₂, —O(C₁-C₆ alkyl), and —S(C₁-C₆ alkyl), and wherein any two heteroatomic moieties are separated from one another by at least two carbon atoms;
   Z is a C₁-C₂₀ alkyl, wherein any one or more —CH₂— groups of the C₁-C₂₀ alkyl is each optionally replaced independently with —C(═O)—, —CF₂—, or a heteroatomic moiety selected from the group consisting of —O—, —S—, —NH—, and —N(C₁-C₆ alkyl)-, wherein any one or more —CH₃ groups of the C₁-C₂₀ alkyl is each optionally replaced independently with —CF₃, —CF₂H, —CH₂F, or a heteroatomic moiety selected from the group consisting of —NH(C₁-C₆ alkyl), —N(C₁-C₆ alkyl)₂, —O(C₁-C₆ alkyl), and —S(C₁-C₆ alkyl), and wherein any two heteroatomic moieties are separated from one another by at least two carbon atoms; and
   B is an oligonucleotide, a polypeptide, a protein, a small molecule, a lipid, a carbohydrate, a polyethylene glycol (PEG) molecule, or a biopolymer,
wherein Y is covalently attached to the 3′-end of A through a first linkage,
the method comprising the steps of
(i) reacting the thiol of formula (VI) with one of the two maleimides of formula (VIII) to form a compound of formula (IX)

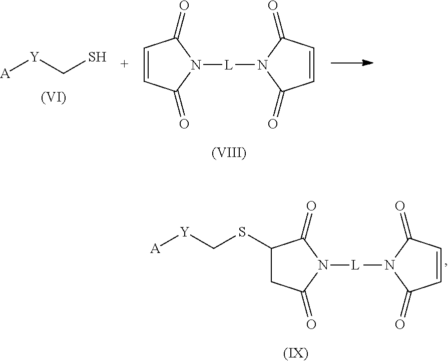

wherein A and Y of formula (VI) are defined as above for formula (V), and wherein L of formula (VIII) is defined as above for formula (V),
(ii) reacting the maleimide of formula (IX) with the thiol of formula (X) to form a compound of formula (II)

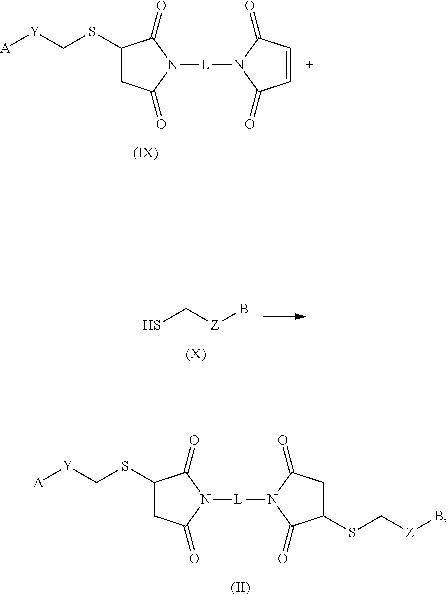

wherein Z and B of formula (X) are as defined above for formula (V), and
(iii) hydrolyzing both of the succinimides of formula (II) to form the oligonucleotide bioconjugate of formula (V)

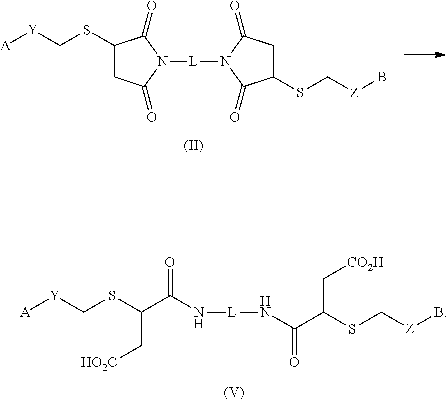

Embodiment 198. The method of Embodiment 197, wherein the first linkage is selected from the group consisting of a phosphate linkage, a phosphorothioate linkage, a phosphoramidate linkage, an amine linkage, an amide linkage, a triazole linkage, an ether linkage, and a thioether linkage.

Embodiment 199. The method of Embodiment 197 or Embodiment 198, wherein the first linkage is a phosphate linkage.

Embodiment 200. The method of Embodiment 199, wherein the phosphate linkage between Y and A comprises a phosphate linked to the 3′-oxygen atom of a nucleotide located at the 3′-end of A.

Embodiment 201. The method of any one of Embodiments 197 to 200, wherein B is an oligonucleotide having a 3′-end and a 5′-end.

Embodiment 202. The method of Embodiment 201, wherein Z is covalently attached to the 3′-end of B through a second linkage selected from the group consisting of a phosphate linkage, a phosphorothioate linkage, a phosphoramidate linkage, an amine linkage, an amide linkage, a triazole linkage, an ether linkage, and a thioether linkage.

Embodiment 203. The method of Embodiment 202, wherein the second linkage is a phosphate linkage.

Embodiment 204. The method of Embodiment 203, wherein the phosphate linkage between Z and B comprises a phosphate linked to the 3′-oxygen atom of a nucleotide located at the 3′-end of B.

Embodiment 205. A method of co-expressing a first polypeptide and a second polypeptide in a cell, the method comprising a step of contacting the cell with an mRNA bioconjugate of formula (I)

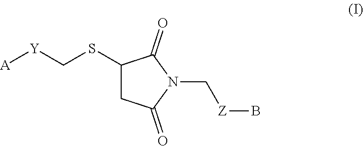

or a pharmaceutically acceptable salt thereof,
wherein
   A is a first mRNA molecule having a 3′-end and a 5′-end, wherein A encodes the first polypeptide;
   Y is a C₁-C₂₀ alkyl, wherein any one or more —CH₂— groups of the C₁-C₂₀ alkyl is each optionally replaced independently with —C(═O)—, —CF₂—, or a heteroatomic moiety selected from the group consisting of —O—, —S—, —NH—, and —N(C₁-C₆ alkyl)-, wherein any one or more —CH₃ groups of the C₁-C₂₀ alkyl is each optionally replaced independently with —CF₃, —CF₂H, —CH₂F, or a heteroatomic moiety selected from the group consisting of —NH(C₁-C₆ alkyl), —N(C₁-C₆ alkyl)₂, —O(C₁-C₆ alkyl), and —S(C₁-C₆ alkyl), and wherein any two heteroatomic moieties are separated from one another by at least two carbon atoms;
   Z is a C₁-C₂₀ alkyl, wherein any one or more —CH₂— groups of the C₁-C₂₀ alkyl is each optionally replaced independently with —C(═O)—, —CF₂—, or a heteroatomic moiety selected from the group consisting of —O—, —S—, —NH—, and —N(C₁-C₆ alkyl)-, wherein any one or more —CH₃ groups of the C₁-C₂₀ alkyl is each optionally replaced independently with —CF₃, —CF₂H, —CH₂F, or a heteroatomic moiety selected from the group consisting of —NH(C₁-C₆ alkyl), —N(C₁-C₆ alkyl)₂, —O(C₁-C₆ alkyl), and —S(C₁-C₆ alkyl), and wherein any two heteroatomic moieties are separated from one another by at least two carbon atoms; and
   B is a second mRNA molecule having a 3′-end and a 5′-end, wherein B encodes the second polypeptide,
wherein Y is covalently attached to the 3′-end of A through a first linkage, and wherein Z is covalently attached to the 3′-end of B through a second linkage.

Embodiment 206. The method of Embodiment 205, wherein the first polypeptide and the second polypeptide are different.

Embodiment 207. The method of Embodiment 205, wherein the first polypeptide and the second polypeptide are the same.

Embodiment 208. The method of any one of Embodiments 205 to 207, wherein the first linkage is selected from the group consisting of a phosphate linkage, a phosphorothioate linkage, a phosphoramidate linkage, an amine linkage, an amide linkage, a triazole linkage, an ether linkage, and a thioether linkage.

Embodiment 209. The method of any one of Embodiments 205 to 208, wherein the second linkage is selected from the group consisting of a phosphate linkage, a phosphorothioate linkage, a phosphoramidate linkage, an amine linkage, an amide linkage, a triazole linkage, an ether linkage, and a thioether linkage.

Embodiment 210. A method of co-expressing a first polypeptide and a second polypeptide in a cell, the method comprising a step of contacting the cell with an mRNA bioconjugate of formula (II)

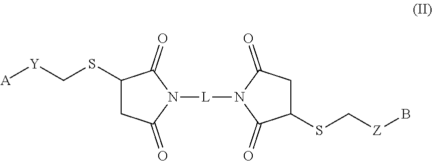

or a pharmaceutically acceptable salt thereof,
wherein
   A is a first mRNA molecule having a 3′-end and a 5′-end, wherein A encodes the first polypeptide;
   Y is a C₁-C₂₀ alkyl, wherein any one or more —CH₂— groups of the C₁-C₂₀ alkyl is each optionally replaced independently with —C(═O)—, —CF₂—, or a heteroatomic moiety selected from the group consisting of —O—, —S—, —NH—, and —N(C₁-C₆ alkyl)-, wherein any one or more —CH₃ groups of the C₁-C₂₀ alkyl is each optionally replaced independently with —CF₃, —CF₂H, —CH₂F, or a heteroatomic moiety selected from the group consisting of —NH(C₁-C₆ alkyl), —N(C₁-C₆ alkyl)₂, —O(C₁-C₆ alkyl), and —S(C₁-C₆ alkyl), and wherein any two heteroatomic moieties are separated from one another by at least two carbon atoms;
   L is a C₂-C₅₀ alkyl, C₂-C₅₀ alkenyl, C₂-C₅₀ alkynyl, C₃-C₈ cycloalkyl, aryl, heteroaryl, C₄-C₅₀ alkyl-cycloalkyl, C₇-C₅₀ alkyl-aryl, or C₆-C₅₀ alkyl-heteroaryl, wherein any one or more —CH₂— groups of L is each optionally replaced independently with —C(═O)—, —CF₂—, or a heteroatomic moiety selected from the group consisting of —O—, —S—, —NH—, and —N(C₁-C₆ alkyl)-, wherein any one or more —CH₃ groups of L is each optionally replaced independently with —CF₃, —CF₂H, —CH₂F, or a heteroatomic moiety selected from the group consisting of —NH(C₁-C₆ alkyl), —N(C₁-C₆ alkyl)₂, —O(C₁-C₆ alkyl), and —S(C₁-C₆ alkyl), and wherein any two heteroatomic moieties are separated from one another by at least two carbon atoms;
   Z is a C₁-C₂₀ alkyl, wherein any one or more —CH₂— groups of the C₁-C₂₀ alkyl is each optionally replaced independently with —C(═O)—, —CF₂—, or a heteroatomic moiety selected from the group consisting of —O—, —S—, —NH—, and —N(C₁-C₆ alkyl)-, wherein any one or more —CH₃ groups of the C₁-C₂₀ alkyl is each optionally replaced independently with —CF₃, —CF₂H, —CH₂F, or a heteroatomic moiety selected from the group consisting of —NH(C₁-C₆ alkyl), —N(C₁-C₆ alkyl)₂, —O(C₁-C₆ alkyl), and —S(C₁-C₆ alkyl), and wherein any two heteroatomic moieties are separated from one another by at least two carbon atoms; and
   B is a second mRNA molecule having a 3′-end and a 5′-end, wherein B encodes the second polypeptide;
wherein Y is covalently attached to the 3′-end of A through a first linkage, and wherein Z is covalently attached to the 3′-end of B through a second linkage.

Embodiment 211. The method of Embodiment 210, wherein the first polypeptide and the second polypeptide are different.

Embodiment 212. The method of Embodiment 210, wherein the first polypeptide and the second polypeptide are identical.

Embodiment 213. The method of any one of Embodiments 210 to 212, wherein the first linkage is selected from the group consisting of a phosphate linkage, a phosphorothioate linkage, a phosphoramidate linkage, an amine linkage, an amide linkage, a triazole linkage, an ether linkage, and a thioether linkage.

Embodiment 214. The method of any one of Embodiments 210 to 213, wherein the second linkage is selected from the group consisting of a phosphate linkage, a phosphorothioate linkage, a phosphoramidate linkage, an amine linkage, an amide linkage, a triazole linkage, an ether linkage, and a thioether linkage.

Embodiment 215. A method of delivering equimolar amounts of a first mRNA molecule and a second mRNA molecule to a cell, the method comprising a step of contacting the cell with an mRNA bioconjugate of formula (I)

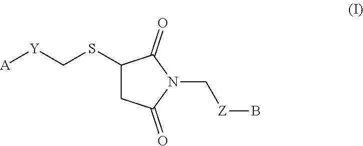

or a pharmaceutically acceptable salt thereof,
wherein
   A is a first mRNA molecule having a 3′-end and a 5′-end;
   Y is a C₁-C₂₀ alkyl, wherein any one or more —CH₂— groups of the C₁-C₂₀ alkyl is each optionally replaced independently with —C(═O)—, —CF₂—, or a heteroatomic moiety selected from the group consisting of —O—, —S—, —NH—, and —N(C₁-C₆ alkyl)-, wherein any one or more —CH₃ groups of the C₁-C₂₀ alkyl is each optionally replaced independently with —CF₃, —CF₂H, —CH₂F, or a heteroatomic moiety selected from the group consisting of —NH(C₁-C₆ alkyl), —N(C₁-C₆ alkyl)₂, —O(C₁-C₆ alkyl), and —S(C₁-C₆ alkyl), and wherein any two heteroatomic moieties are separated from one another by at least two carbon atoms;
   Z is a C₁-C₂₀ alkyl, wherein any one or more —CH₂— groups of the C₁-C₂₀ alkyl is each optionally replaced independently with —C(═O)—, —CF₂—, or a heteroatomic moiety selected from the group consisting of —O—, —S—, —NH—, and —N(C₁-C₆ alkyl)-, wherein any one or more —CH₃ groups of the C₁-C₂₀ alkyl is each optionally replaced independently with —CF₃, —CF₂H, —CH₂F, or a heteroatomic moiety selected from the group consisting of —NH(C₁-C₆ alkyl), —N(C₁-C₆ alkyl)₂, —O(C₁-C₆ alkyl), and —S(C₁-C₆ alkyl), and wherein any two heteroatomic moieties are separated from one another by at least two carbon atoms; and
   B is a second mRNA molecule having a 3′-end and a 5′-end,
wherein Y is covalently attached to the 3′-end of A through a first linkage, and wherein Z is covalently attached to the 3′-end of B through a second linkage.

Embodiment 216. The method of Embodiment 215, wherein the first mRNA molecule and the second mRNA molecule are different.

Embodiment 217. The method of Embodiment 215, wherein the first polypeptide and the second polypeptide are identical.

Embodiment 218. The method of any one of Embodiments 215 to 217, wherein the first linkage is selected from the group consisting of a phosphate linkage, a phosphorothioate linkage, a phosphoramidate linkage, an amine linkage, an amide linkage, a triazole linkage, an ether linkage, and a thioether linkage.

Embodiment 219. The method of any one of Embodiments 215 to 218, wherein the second linkage is selected from the group consisting of a phosphate linkage, a phosphorothioate linkage, a phosphoramidate linkage, an amine linkage, an amide linkage, a triazole linkage, an ether linkage, and a thioether linkage.

Embodiment 220. A method of delivering equimolar amounts of a first mRNA molecule and a second mRNA molecule to a cell, the method comprising a step of contacting the cell with an mRNA bioconjugate of formula (II)

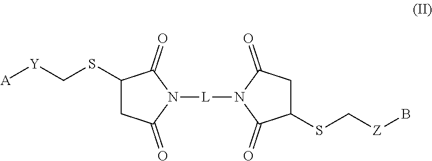

or a pharmaceutically acceptable salt thereof,
wherein
   A is a first mRNA molecule having a 3′-end and a 5′-end;
   Y is a C₁-C₂₀ alkyl, wherein any one or more —CH₂— groups of the C₁-C₂₀ alkyl is each optionally replaced independently with —C(═O)—, —CF₂—, or a heteroatomic moiety selected from the group consisting of —O—, —S—, —NH—, and —N(C₁-C₆ alkyl)-, wherein any one or more —CH₃ groups of the C₁-C₂₀ alkyl is each optionally replaced independently with —CF₃, —CF₂H, —CH₂F, or a heteroatomic moiety selected from the group consisting of —NH(C₁-C₆ alkyl), —N(C₁-C₆ alkyl)₂, —O(C₁-C₆ alkyl), and —S(C₁-C₆ alkyl), and wherein any two heteroatomic moieties are separated from one another by at least two carbon atoms;
   L is a C₂-C₅₀ alkyl, C₂-C₅₀ alkenyl, C₂-C₅₀ alkynyl, C₃-C₈ cycloalkyl, aryl, heteroaryl, C₄-C₅₀ alkyl-cycloalkyl, C₇-C₅₀ alkyl-aryl, or C₆-C₅₀ alkyl-heteroaryl, wherein any one or more —CH₂— groups of L is each optionally replaced independently with —C(═O)—, —CF₂—, or a heteroatomic moiety selected from the group consisting of —O—, —S—, —NH—, and —N(C₁-C₆ alkyl)-, wherein any one or more —CH₃ groups of L is each optionally replaced independently with —CF₃, —CF₂H, —CH₂F, or a heteroatomic moiety selected from the group consisting of —NH(C₁-C₆ alkyl), —N(C₁-C₆ alkyl)₂, —O(C₁-C₆ alkyl), and —S(C₁-C₆ alkyl), and wherein any two heteroatomic moieties are separated from one another by at least two carbon atoms;
   Z is a C₁-C₂₀ alkyl, wherein any one or more —CH₂— groups of the C₁-C₂₀ alkyl is each optionally replaced independently with —C(═O)—, —CF₂—, or a heteroatomic moiety selected from the group consisting of —O—, —S—, —NH—, and —N(C₁-C₆ alkyl)-, wherein any one or more —CH₃ groups of the C₁-C₂₀ alkyl is each optionally replaced independently with —CF₃, —CF₂H, —CH₂F, or a heteroatomic moiety selected from the group consisting of —NH(C₁-C₆ alkyl), —N(C₁-C₆ alkyl)₂, —O(C₁-C₆ alkyl), and —S(C₁-C₆ alkyl), and wherein any two heteroatomic moieties are separated from one another by at least two carbon atoms; and
   B is a second mRNA molecule having a 3′-end and a 5′-end;
wherein Y is covalently attached to the 3′-end of A through a first linkage, and wherein Z is covalently attached to the 3′-end of B through a second linkage.

Embodiment 221. The method of Embodiment 220, wherein the first mRNA molecule and the second mRNA molecule are different.

Embodiment 222. The method of Embodiment 220, wherein the first mRNA molecule and the second mRNA molecule are identical.

Embodiment 223. The method of any one of Embodiments 220 to 222, wherein the first linkage is selected from the group consisting of a phosphate linkage, a phosphorothioate linkage, a phosphoramidate linkage, an amine linkage, an amide linkage, a triazole linkage, an ether linkage, and a thioether linkage.

Embodiment 224. The method of any one of Embodiments 220 to 223, wherein the second linkage is selected from the group consisting of a phosphate linkage, a phosphorothioate linkage, a phosphoramidate linkage, an amine linkage, an amide linkage, a triazole linkage, an ether linkage, and a thioether linkage.

Embodiment 225. A method of targeted therapy, the method comprising a step of administering to a patient in need thereof a pharmaceutical formulation comprising an mRNA bioconjugate of formula (I)

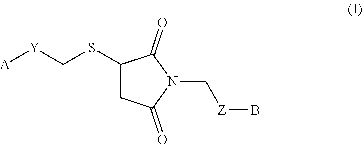

or a pharmaceutically acceptable salt thereof, and a pharmaceutically acceptable carrier, excipient, or diluent,
wherein
   A is a first mRNA molecule having a 3′-end and a 5′-end, wherein A encodes a first therapeutic polypeptide;
   Y is a C₁-C₂₀ alkyl, wherein any one or more —CH₂— groups of the C₁-C₂₀ alkyl is each optionally replaced independently with —C(═O)—, —CF₂—, or a heteroatomic moiety selected from the group consisting of —O—, —S—, —NH—, and —N(C₁-C₆ alkyl)-, wherein any one or more —CH₃ groups of the C₁-C₂₀ alkyl is each optionally replaced independently with —CF₃, —CF₂H, —CH₂F, or a heteroatomic moiety selected from the group consisting of —NH(C₁-C₆ alkyl), —N(C₁-C₆ alkyl)₂, —O(C₁-C₆ alkyl), and —S(C₁-C₆ alkyl), and wherein any two heteroatomic moieties are separated from one another by at least two carbon atoms;
   Z is a C₁-C₂₀ alkyl, wherein any one or more —CH₂— groups of the C₁-C₂₀ alkyl is each optionally replaced independently with —C(═O)—, —CF₂—, or a heteroatomic moiety selected from the group consisting of —O—, —S—, —NH—, and —N(C₁-C₆ alkyl)-, wherein any one or more —CH₃ groups of the C₁-C₂₀ alkyl is each optionally replaced independently with —CF₃, —CF₂H, —CH₂F, or a heteroatomic moiety selected from the group consisting of —NH(C₁-C₆ alkyl), —N(C₁-C₆ alkyl)₂, —O(C₁-C₆ alkyl), and —S(C₁-C₆ alkyl), and wherein any two heteroatomic moieties are separated from one another by at least two carbon atoms; and
   B is a second therapeutic polypeptide, a therapeutic small molecule, or a second mRNA molecule having a 3′-end and a 5′-end encoding a second therapeutic polypeptide,
wherein Y is covalently attached to the 3′-end of A through a first linkage.

Embodiment 226. The method of Embodiment 225, wherein the first linkage is selected from the group consisting of a phosphate linkage, a phosphorothioate linkage, a phosphoramidate linkage, an amine linkage, an amide linkage, a triazole linkage, an ether linkage, and a thioether linkage.

Embodiment 227. The method of Embodiment 225 or Embodiment 226, wherein the targeted therapy delivers the mRNA bioconjugate to a specific cell type, organ, tumor, or anatomical location in the patient in need thereof.

Embodiment 228. The method of any one of Embodiments 225 to 227, wherein B is a second mRNA molecule, and wherein Z is covalently attached to the 3′-end of B through a second linkage.

Embodiment 229. The method of Embodiment 228, wherein the second linkage is selected from the group consisting of a phosphate linkage, a phosphorothioate linkage, a phosphoramidate linkage, an amine linkage, an amide linkage, a triazole linkage, an ether linkage, and a thioether linkage.

Embodiment 230. The method of Embodiment 228 or Embodiment 229, further comprising a step of co-expressing the first and second therapeutic polypeptides.

Embodiment 231. The method of Embodiment 230, wherein the first and second therapeutic polypeptides are co-expressed in the specific cell type, organ, tumor, or anatomical location to which the mRNA bioconjugate has been delivered.

Embodiment 232. The method of any one of Embodiments 225 to 227, wherein B is a second therapeutic polypeptide.

Embodiment 233. The method of Embodiment 232, wherein B is a therapeutic antibody or a therapeutic enzyme.

Embodiment 234. The method of any one of Embodiments 225 to 227, wherein B is a therapeutic small molecule.

Embodiment 235. The method of any one of Embodiments 232 to 234, further comprising a step of expressing the first therapeutic polypeptide.

Embodiment 236. The method of Embodiment 235, wherein the first therapeutic polypeptide is expressed in the specific cell type, organ, tumor, or anatomical location to which the mRNA bioconjugate has been delivered.

Embodiment 237. The method of any one of Embodiments 225 to 236, wherein the administering is by intravenous administration, subcutaneous administration, infusion, intramuscular administration, or oral administration.

Embodiment 238. The method of any one of Embodiments 225 to 237, wherein the pharmaceutically acceptable carrier, excipient, or diluent comprises a lipid-based carrier, a polymer-based carrier, or a nanocarrier.

Embodiment 239. The method of any one of Embodiments 225 to 238, wherein the targeted therapy is targeted cancer therapy.

Embodiment 240. The method of any one of Embodiments 225 to 238, wherein the targeted therapy is a targeted obesity therapy.

Embodiment 241. The method of any one of Embodiments 225 to 240, wherein the patient in need thereof is a human.

Embodiment 242. A method of targeted therapy, the method comprising a step of administering to a patient in need thereof a pharmaceutical formulation comprising an mRNA bioconjugate of formula (II)

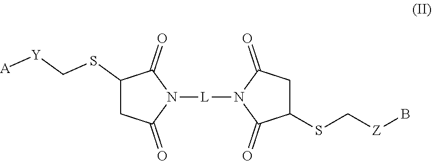

or a pharmaceutically acceptable salt thereof, and a pharmaceutically acceptable carrier, excipient, or diluent,
wherein
   A is a first mRNA molecule having a 3′-end and a 5′-end, wherein A encodes a first therapeutic polypeptide;
   Y is a C₁-C₂₀ alkyl, wherein any one or more —CH₂— groups of the C₁-C₂₀ alkyl is each optionally replaced independently with —C(═O)—, —CF₂—, or a heteroatomic moiety selected from the group consisting of —O—, —S—, —NH—, and —N(C₁-C₆ alkyl)-, wherein any one or more —CH₃ groups of the C₁-C₂₀ alkyl is each optionally replaced independently with —CF₃, —CF₂H, —CH₂F, or a heteroatomic moiety selected from the group consisting of —NH(C₁-C₆ alkyl), —N(C₁-C₆ alkyl)₂, —O(C₁-C₆ alkyl), and —S(C₁-C₆ alkyl), and wherein any two heteroatomic moieties are separated from one another by at least two carbon atoms;
   L is a C₂-C₅₀ alkyl, C₂-C₅₀ alkenyl, C₂-C₅₀ alkynyl, C₃-C₈ cycloalkyl, aryl, heteroaryl, C₄-C₅₀ alkyl-cycloalkyl, C₇-C₅₀ alkyl-aryl, or C₆-C₅₀ alkyl-heteroaryl, wherein any one or more —CH₂— groups of L is each optionally replaced independently with —C(═O)—, —CF₂—, or a heteroatomic moiety selected from the group consisting of —O—, —S—, —NH—, and —N(C₁-C₆ alkyl)-, wherein any one or more —CH₃ groups of L is each optionally replaced independently with —CF₃, —CF₂H, —CH₂F, or a heteroatomic moiety selected from the group consisting of —NH(C₁-C₆ alkyl), —N(C₁-C₆ alkyl)₂, —O(C₁-C₆ alkyl), and —S(C₁-C₆ alkyl), and wherein any two heteroatomic moieties are separated from one another by at least two carbon atoms;
   Z is a C₁-C₂₀ alkyl, wherein any one or more —CH₂— groups of the C₁-C₂₀ alkyl is each optionally replaced independently with —C(═O)—, —CF₂—, or a heteroatomic moiety selected from the group consisting of —O—, —S—, —NH—, and —N(C₁-C₆ alkyl)-, wherein any one or more —CH₃ groups of the C₁-C₂₀ alkyl is each optionally replaced independently with —CF₃, —CF₂H, —CH₂F, or a heteroatomic moiety selected from the group consisting of —NH(C₁-C₆ alkyl), —N(C₁-C₆ alkyl)₂, —O(C₁-C₆ alkyl), and —S(C₁-C₆ alkyl), and wherein any two heteroatomic moieties are separated from one another by at least two carbon atoms; and
   B is a second therapeutic polypeptide, a therapeutic small molecule, or a second mRNA molecule having a 3′-end and a 5′-end encoding a second therapeutic polypeptide,
wherein Y is covalently attached to the 3′-end of A through a first linkage.

Embodiment 243. The method of Embodiment 242, wherein the first linkage is selected from the group consisting of a phosphate linkage, a phosphorothioate linkage, a phosphoramidate linkage, an amine linkage, an amide linkage, a triazole linkage, an ether linkage, and a thioether linkage.

Embodiment 244. The method of Embodiment 242 or Embodiment 243, wherein the targeted therapy delivers the mRNA bioconjugate to a specific cell type, organ, tumor, or anatomical location in the patient in need thereof.

Embodiment 245. The method of any one of Embodiments 242 to 244, wherein B is a second mRNA molecule, and wherein Z is covalently attached to the 3′-end of B through a second linkage.

Embodiment 246. The method of Embodiment 245, wherein the second linkage is selected from the group consisting of a phosphate linkage, a phosphorothioate linkage, a phosphoramidate linkage, an amine linkage, an amide linkage, a triazole linkage, an ether linkage, and a thioether linkage.

Embodiment 247. The method of Embodiment 245 or Embodiment 246, further comprising a step of co-expressing the first and second therapeutic polypeptides.

Embodiment 248. The method of Embodiment 247, wherein the first and second therapeutic polypeptides are co-expressed in the specific cell type, organ, tumor, or anatomical location to which the mRNA bioconjugate has been delivered.

Embodiment 249. The method of any one of Embodiments 242 to 244, wherein B is a second therapeutic polypeptide.

Embodiment 250. The method of Embodiment 249, wherein B is a therapeutic antibody or a therapeutic enzyme

Embodiment 251. The method of any one of Embodiments 242 to 244, wherein B is a therapeutic small molecule.

Embodiment 252. The method of any one of Embodiments 249 to 251, further comprising a step of expressing the first therapeutic polypeptide.

Embodiment 253. The method of Embodiment 252, wherein the first therapeutic polypeptide is expressed in the specific cell type, organ, tumor, or anatomical location to which the mRNA bioconjugate has been delivered.

Embodiment 254. The method of any one of Embodiments 242 to 253, wherein the administering is by intravenous administration, subcutaneous administration, infusion, intramuscular administration, or oral administration.

Embodiment 255. The method of any one of Embodiments 242 to 254, wherein the pharmaceutically acceptable carrier, excipient, or diluent comprises a lipid-based carrier, a polymer-based carrier, or a nanocarrier.

Embodiment 256. The method of any one of Embodiments 242 to 255, wherein the targeted therapy is targeted cancer therapy.

Embodiment 257. The method of any one of Embodiments 242 to 255, wherein the targeted therapy is a targeted obesity therapy.

Embodiment 258. The method of any one of Embodiments 242 to 257, wherein the patient in need thereof is a human.

Embodiment 259. A method of treating, preventing, slowing the progression, or reducing the severity of cancer in a patient in need thereof, the method comprising a step of administering to the patient in need thereof a pharmaceutical formulation comprising an mRNA bioconjugate of formula (I) A,)

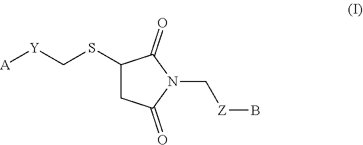

or a pharmaceutically acceptable salt thereof, and a pharmaceutically acceptable carrier, excipient, or diluent,
wherein
   A is a first mRNA molecule having a 3′-end and a 5′-end, wherein A encodes a first therapeutic polypeptide;
   Y is a C₁-C₂₀ alkyl, wherein any one or more —CH₂— groups of the C₁-C₂₀ alkyl is each optionally replaced independently with —C(═O)—, —CF₂—, or a heteroatomic moiety selected from the group consisting of —O—, —S—, —NH—, and —N(C₁-C₆ alkyl)-, wherein any one or more —CH₃ groups of the C₁-C₂₀ alkyl is each optionally replaced independently with —CF₃, —CF₂H, —CH₂F, or a heteroatomic moiety selected from the group consisting of —NH(C₁-C₆ alkyl), —N(C₁-C₆ alkyl)₂, —O(C₁-C₆ alkyl), and —S(C₁-C₆ alkyl), and wherein any two heteroatomic moieties are separated from one another by at least two carbon atoms;
   Z is a C₁-C₂₀ alkyl, wherein any one or more —CH₂— groups of the C₁-C₂₀ alkyl is each optionally replaced independently with —C(═O)—, —CF₂—, or a heteroatomic moiety selected from the group consisting of —O—, —S—, —NH—, and —N(C₁-C₆ alkyl)-, wherein any one or more —CH₃ groups of the C₁-C₂₀ alkyl is each optionally replaced independently with —CF₃, —CF₂H, —CH₂F, or a heteroatomic moiety selected from the group consisting of —NH(C₁-C₆ alkyl), —N(C₁-C₆ alkyl)₂, —O(C₁-C₆ alkyl), and —S(C₁-C₆ alkyl), and wherein any two heteroatomic moieties are separated from one another by at least two carbon atoms; and
   B is a second therapeutic polypeptide, a therapeutic small molecule, or a second mRNA molecule having a 3′-end and a 5′-end encoding a second therapeutic polypeptide,
wherein Y is covalently attached to the 3′-end of A through a first linkage.

Embodiment 260. The method of Embodiment 259, wherein the first linkage is selected from the group consisting of a phosphate linkage, a phosphorothioate linkage, a phosphoramidate linkage, an amine linkage, an amide linkage, a triazole linkage, an ether linkage, and a thioether linkage.

Embodiment 261. The method of Embodiment 259 or Embodiment 260, wherein B is a second mRNA molecule, and wherein Z is covalently attached to the 3′-end of B through a second linkage.

Embodiment 262. The method of Embodiment 261, wherein the second linkage is selected from the group consisting of a phosphate linkage, a phosphorothioate linkage, a phosphoramidate linkage, an amine linkage, an amide linkage, a triazole linkage, an ether linkage, and a thioether linkage.

Embodiment 263. The method of Embodiment 259 or Embodiment 260, wherein B is a second therapeutic polypeptide.

Embodiment 264. The method of Embodiment 263, wherein B is a therapeutic antibody or a therapeutic enzyme.

Embodiment 265. The method of Embodiment 259 or Embodiment 260, wherein B is a therapeutic small molecule.

Embodiment 266. The method of any one of Embodiments 259 to 265, wherein the administering is by intravenous administration, subcutaneous administration, infusion, intramuscular administration, or oral administration.

Embodiment 267. The method of any one of Embodiments 259 to 266, wherein the pharmaceutically acceptable carrier, excipient, or diluent comprises a lipid-based carrier, a polymer-based carrier, or a nanocarrier.

Embodiment 268. The method of any one of Embodiments 259 to 267, wherein the cancer is selected from the group consisting of breast cancer, lung cancer, pancreatic cancer, and colorectal cancer.

Embodiment 269. The method of any one of Embodiments 259 to 268, wherein the patient in need thereof is a human.

Embodiment 270. A method of treating, preventing, slowing the progression, or reducing the severity of cancer in a patient in need thereof, the method comprising a step of administering to the patient in need thereof a pharmaceutical formulation comprising an mRNA bioconjugate of formula (II)

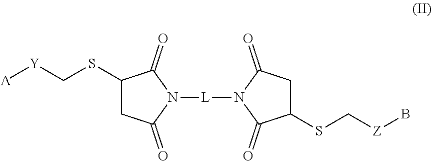

or a pharmaceutically acceptable salt thereof, and a pharmaceutically acceptable carrier, excipient, or diluent,
wherein
   A is a first mRNA molecule having a 3′-end and a 5′-end, wherein A encodes a first therapeutic polypeptide;
   Y is a C₁-C₂₀ alkyl, wherein any one or more —CH₂— groups of the C₁-C₂₀ alkyl is each optionally replaced independently with —C(═O)—, —CF₂—, or a heteroatomic moiety selected from the group consisting of —O—, —S—, —NH—, and —N(C₁-C₆ alkyl)-, wherein any one or more —CH₃ groups of the C₁-C₂₀ alkyl is each optionally replaced independently with —CF₃, —CF₂H, —CH₂F, or a heteroatomic moiety selected from the group consisting of —NH(C₁-C₆ alkyl), —N(C₁-C₆ alkyl)₂, —O(C₁-C₆ alkyl), and —S(C₁-C₆ alkyl), and wherein any two heteroatomic moieties are separated from one another by at least two carbon atoms;
   L is a C₂-C₅₀ alkyl, C₂-C₅₀ alkenyl, C₂-C₅₀ alkynyl, C₃-C₅ cycloalkyl, aryl, heteroaryl, C₄-C₅₀ alkyl-cycloalkyl, C₇-C₅₀ alkyl-aryl, or C₆-C₅₀ alkyl-heteroaryl, wherein any one or more —CH₂— groups of the C₂-C₅₀ alkyl is each optionally replaced independently with —C(═O)—, —CF₂—, or a heteroatomic moiety selected from the group consisting of —O—, —S—, —NH—, and —N(C₁-C₆ alkyl)-, wherein any one or more —CH₃ groups of L is each optionally replaced independently with —CF₃, —CF₂H, —CH₂F, or a heteroatomic moiety selected from the group consisting of —NH(C₁-C₆ alkyl), —N(C₁-C₆ alkyl)₂, —O(C₁-C₆ alkyl), and —S(C₁-C₆ alkyl), and wherein any two heteroatomic moieties are separated from one another by at least two carbon atoms;
   Z is a C₁-C₂₀ alkyl, wherein any one or more —CH₂— groups of the C₁-C₂₀ alkyl is each optionally replaced independently with —C(═O)—, —CF₂—, or a heteroatomic moiety selected from the group consisting of —O—, —S—, —NH—, and —N(C₁-C₆ alkyl)-, wherein any one or more —CH₃ groups of the C₁-C₂₀ alkyl is each optionally replaced independently with —CF₃, —CF₂H, —CH₂F, or a heteroatomic moiety selected from the group consisting of —NH(C₁-C₆ alkyl), —N(C₁-C₆ alkyl)₂, —O(C₁-C₆ alkyl), and —S(C₁-C₆ alkyl), and wherein any two heteroatomic moieties are separated from one another by at least two carbon atoms; and
   B is a second therapeutic polypeptide, a therapeutic small molecule, or a second mRNA molecule having a 3′-end and a 5′-end encoding a second therapeutic polypeptide,
wherein Y is covalently attached to the 3′-end of A through a first linkage.

Embodiment 271. The method of Embodiment 270, wherein the first linkage is selected from the group consisting of a phosphate linkage, a phosphorothioate linkage, a phosphoramidate linkage, an amine linkage, an amide linkage, a triazole linkage, an ether linkage, and a thioether linkage.

Embodiment 272. The method of Embodiment 270 or Embodiment 271, wherein B is a second mRNA molecule, and wherein Z is covalently attached to the 3′-end of B through a second linkage.

Embodiment 273. The method of Embodiment 272, wherein the second linkage is selected from the group consisting of a phosphate linkage, a phosphorothioate linkage, a phosphoramidate linkage, an amine linkage, an amide linkage, a triazole linkage, an ether linkage, and a thioether linkage.

Embodiment 274. The method of Embodiment 270 or Embodiment 271, wherein B is a second therapeutic polypeptide.

Embodiment 275. The method of Embodiment 274, wherein B is a therapeutic antibody or a therapeutic enzyme.

Embodiment 276. The method of Embodiment 270 or Embodiment 271, wherein B is a therapeutic small molecule.

Embodiment 277. The method of any one of Embodiments 270 to 276, wherein the administering is by intravenous administration, subcutaneous administration, infusion, intramuscular administration, or oral administration.

Embodiment 278. The method of any one of Embodiments 270 to 277, wherein the pharmaceutically acceptable carrier, excipient, or diluent comprises a lipid-based carrier, a polymer-based carrier, or a nanocarrier.

Embodiment 279. The method of any one of Embodiments 270 to 278, wherein the cancer is selected from the group consisting of breast cancer, lung cancer, pancreatic cancer, and colorectal cancer.

Embodiment 280. The method of any one of Embodiments 270 to 279, wherein the patient in need thereof is a human.

Embodiment 281. A method of treating, preventing, slowing the progression, or reducing the severity of obesity in a patient in need thereof, the method comprising a step of administering to the patient in need thereof a pharmaceutical formulation comprising an mRNA bioconjugate of formula (I)

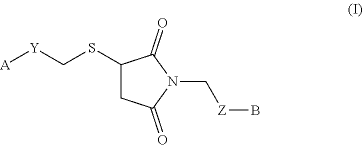

or a pharmaceutically acceptable salt thereof, and a pharmaceutically acceptable carrier, excipient, or diluent,
wherein
   A is a first mRNA molecule having a 3′-end and a 5′-end, wherein A encodes a first therapeutic polypeptide;
   Y is a C₁-C₂₀ alkyl, wherein any one or more —CH₂— groups of the C₁-C₂₀ alkyl is each optionally replaced independently with —C(═O)—, —CF₂—, or a heteroatomic moiety selected from the group consisting of —O—, —S—, —NH—, and —N(C₁-C₆ alkyl)-, wherein any one or more —CH₃ groups of the C₁-C₂₀ alkyl is each optionally replaced independently with —CF₃, —CF₂H, —CH₂F, or a heteroatomic moiety selected from the group consisting of —NH(C₁-C₆ alkyl), —N(C₁-C₆ alkyl)₂, —O(C₁-C₆ alkyl), and —S(C₁-C₆ alkyl), and wherein any two heteroatomic moieties are separated from one another by at least two carbon atoms;
   Z is a C₁-C₂₀ alkyl, wherein any one or more —CH₂— groups of the C₁-C₂₀ alkyl is each optionally replaced independently with —C(═O)—, —CF₂—, or a heteroatomic moiety selected from the group consisting of —O—, —S—, —NH—, and —N(C₁-C₆ alkyl)-, wherein any one or more —CH₃ groups of the C₁-C₂₀ alkyl is each optionally replaced independently with —CF₃, —CF₂H, —CH₂F, or a heteroatomic moiety selected from the group consisting of —NH(C₁-C₆ alkyl), —N(C₁-C₆ alkyl)₂, —O(C₁-C₆ alkyl), and —S(C₁-C₆ alkyl), and wherein any two heteroatomic moieties are separated from one another by at least two carbon atoms; and
   B is a second therapeutic polypeptide, a therapeutic small molecule, or a second mRNA molecule having a 3′-end and a 5′-end encoding a second therapeutic polypeptide,
wherein Y is covalently attached to the 3′-end of A through a first linkage.

Embodiment 282. The method of Embodiment 281, wherein the first linkage is selected from the group consisting of a phosphate linkage, a phosphorothioate linkage, a phosphoramidate linkage, an amine linkage, an amide linkage, a triazole linkage, an ether linkage, and a thioether linkage.

Embodiment 283. The method of Embodiment 281 or Embodiment 282, wherein B is a second mRNA molecule, and wherein Z is covalently attached to the 3′-end of B through a second linkage.

Embodiment 284. The method of Embodiment 283, wherein the second linkage is selected from the group consisting of a phosphate linkage, a phosphorothioate linkage, a phosphoramidate linkage, an amine linkage, an amide linkage, a triazole linkage, an ether linkage, and a thioether linkage.

Embodiment 285. The method of Embodiment 281 or Embodiment 282, wherein B is a second therapeutic polypeptide.

Embodiment 286. The method of Embodiment 285, wherein B is a therapeutic antibody or a therapeutic enzyme.

Embodiment 287. The method of Embodiment 281 or Embodiment 282, wherein B is a therapeutic small molecule.

Embodiment 288. The method of any one of Embodiments 281 to 287, wherein the administering is by intravenous administration, subcutaneous administration, infusion, intramuscular administration, or oral administration.

Embodiment 289. The method of any one of Embodiments 281 to 288, wherein the pharmaceutically acceptable carrier, excipient, or diluent comprises a lipid-based carrier, a polymer-based carrier, or a nanocarrier.

Embodiment 290. The method of any one of Embodiments 281 to 289, wherein the patient in need thereof is a human.

Embodiment 291. A method of treating, preventing, slowing the progression, or reducing the severity of obesity in a patient in need thereof, the method comprising a step of administering to the patient in need thereof a pharmaceutical formulation comprising an mRNA bioconjugate of formula (II)

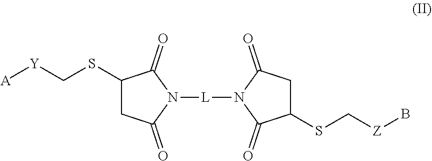

or a pharmaceutically acceptable salt thereof, and a pharmaceutically acceptable carrier, excipient, or diluent,
wherein
   A is a first mRNA molecule having a 3′-end and a 5′-end, wherein A encodes a first therapeutic polypeptide;
   Y is a C₁-C₂₀ alkyl, wherein any one or more —CH₂— groups of the C₁-C₂₀ alkyl is each optionally replaced independently with —C(═O)—, —CF₂—, or a heteroatomic moiety selected from the group consisting of —O—, —S—, —NH—, and —N(C₁-C₆ alkyl)-, wherein any one or more —CH₃ groups of the C₁-C₂₀ alkyl is each optionally replaced independently with —CF₃, —CF₂H, —CH₂F, or a heteroatomic moiety selected from the group consisting of —NH(C₁-C₆ alkyl), —N(C₁-C₆ alkyl)₂, —O(C₁-C₆ alkyl), and —S(C₁-C₆ alkyl), and wherein any two heteroatomic moieties are separated from one another by at least two carbon atoms;
   L is a C₂-C₅₀ alkyl, C₂-C₅₀ alkenyl, C₂-C₅₀ alkynyl, C₃-C₈ cycloalkyl, aryl, heteroaryl, C₄-C₅₀ alkyl-cycloalkyl, C₇-C₅₀ alkyl-aryl, or C₆-C₅₀ alkyl-heteroaryl, wherein any one or more —CH₂— groups of the C₂-C₅₀ alkyl is each optionally replaced independently with —C(═O)—, —CF₂—, or a heteroatomic moiety selected from the group consisting of —O—, —S—, —NH—, and —N(C₁-C₆ alkyl)-, wherein any one or more —CH₃ groups of L is each optionally replaced independently with —CF₃, —CF₂H, —CH₂F, or a heteroatomic moiety selected from the group consisting of —NH(C₁-C₆ alkyl), —N(C₁-C₆ alkyl)₂, —O(C₁-C₆ alkyl), and —S(C₁-C₆ alkyl), and wherein any two heteroatomic moieties are separated from one another by at least two carbon atoms;
   Z is a C₁-C₂₀ alkyl, wherein any one or more —CH₂— groups of the C₁-C₂₀ alkyl is each optionally replaced independently with —C(═O)—, —CF₂—, or a heteroatomic moiety selected from the group consisting of —O—, —S—, —NH—, and —N(C₁-C₆ alkyl)-, wherein any one or more —CH₃ groups of the C₁-C₂₀ alkyl is each optionally replaced independently with —CF₃, —CF₂H, —CH₂F, or a heteroatomic moiety selected from the group consisting of —NH(C₁-C₆ alkyl), —N(C₁-C₆ alkyl)₂, —O(C₁-C₆ alkyl), and —S(C₁-C₆ alkyl), and wherein any two heteroatomic moieties are separated from one another by at least two carbon atoms; and
   B is a second therapeutic polypeptide, a therapeutic small molecule, or a second mRNA molecule having a 3′-end and a 5′-end encoding a second therapeutic polypeptide,
wherein Y is covalently attached to the 3′-end of A through a first linkage.

Embodiment 292. The method of Embodiment 291, wherein the first linkage is selected from the group consisting of a phosphate linkage, a phosphorothioate linkage, a phosphoramidate linkage, an amine linkage, an amide linkage, a triazole linkage, an ether linkage, and a thioether linkage.

Embodiment 293. The method of Embodiment 291 or Embodiment 292, wherein B is a second mRNA molecule, and wherein Z is covalently attached to the 3′-end of B through a second linkage.

Embodiment 294. The method of Embodiment 293, wherein the second linkage is selected from the group consisting of a phosphate linkage, a phosphorothioate linkage, a phosphoramidate linkage, an amine linkage, an amide linkage, a triazole linkage, an ether linkage, and a thioether linkage.

Embodiment 295. The method of Embodiment 291 or Embodiment 292, wherein B is a second therapeutic polypeptide.

Embodiment 296. The method of Embodiment 295, wherein B is a therapeutic antibody or a therapeutic enzyme.

Embodiment 297. The method of Embodiment 291 or Embodiment 292, wherein B is a therapeutic small molecule.

Embodiment 298. The method of any one of Embodiments 291 to 297, wherein the administering is by intravenous administration, subcutaneous administration, infusion, intramuscular administration, or oral administration.

Embodiment 299. The method of any one of Embodiments 291 to 298, wherein the pharmaceutically acceptable carrier, excipient, or diluent comprises a lipid-based carrier, a polymer-based carrier, or a nanocarrier.

Embodiment 300. The method of any one of Embodiments 291 to 299, wherein the patient in need thereof is a human.

Embodiment 301. A method of enzyme replacement therapy, the method comprising a step of administering to a patient in need of enzyme replacement therapy a pharmaceutical formulation comprising an mRNA bioconjugate of formula (I)

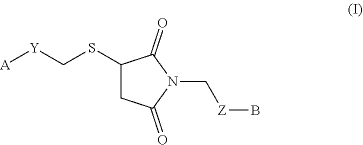

or a pharmaceutically acceptable salt thereof, and a pharmaceutically acceptable carrier, excipient, or diluent,
wherein
   A is a first mRNA molecule having a 3′-end and a 5′-end, wherein A encodes a first enzyme;
   Y is a C₁-C₂₀ alkyl, wherein any one or more —CH₂— groups of the C₁-C₂₀ alkyl is each optionally replaced independently with —C(═O)—, —CF₂—, or a heteroatomic moiety selected from the group consisting of —O—, —S—, —NH—, and —N(C₁-C₆ alkyl)-, wherein any one or more —CH₃ groups of the C₁-C₂₀ alkyl is each optionally replaced independently with —CF₃, —CF₂H, —CH₂F, or a heteroatomic moiety selected from the group consisting of —NH(C₁-C₆ alkyl), —N(C₁-C₆ alkyl)₂, —O(C₁-C₆ alkyl), and —S(C₁-C₆ alkyl), and wherein any two heteroatomic moieties are separated from one another by at least two carbon atoms;
   Z is a C₁-C₂₀ alkyl, wherein any one or more —CH₂— groups of the C₁-C₂₀ alkyl is each optionally replaced independently with —C(═O)—, —CF₂—, or a heteroatomic moiety selected from the group consisting of —O—, —S—, —NH—, and —N(C₁-C₆ alkyl)-, wherein any one or more —CH₃ groups of the C₁-C₂₀ alkyl is each optionally replaced independently with —CF₃, —CF₂H, —CH₂F, or a heteroatomic moiety selected from the group consisting of —NH(C₁-C₆ alkyl), —N(C₁-C₆ alkyl)₂, —O(C₁-C₆ alkyl), and —S(C₁-C₆ alkyl), and wherein any two heteroatomic moieties are separated from one another by at least two carbon atoms; and
   B is an oligonucleotide, a polypeptide, a protein, a second enzyme, a small molecule, a carbohydrate, a lipid, a polyethylene glycol (PEG) molecule, or a biopolymer,
wherein Y is covalently attached to the 3′-end of A through a first linkage, and wherein the patient in need of enzyme replacement therapy is deficient of the first enzyme.

Embodiment 302. The method of Embodiment 301, wherein the first linkage is selected from the group consisting of a phosphate linkage, a phosphorothioate linkage, a phosphoramidate linkage, an amine linkage, an amide linkage, a triazole linkage, an ether linkage, and a thioether linkage.

Embodiment 303. The method of Embodiment 301 or Embodiment 302, wherein B is a second enzyme.

Embodiment 304. The method of Embodiment 303, wherein the patient in need of enzyme replacement therapy is deficient of the second enzyme.

Embodiment 305. The method of Embodiment 301 or Embodiment 302, wherein B is an oligonucleotide having a 3′-end and a 5′-end.

Embodiment 306. The method of Embodiment 305, wherein Z is covalently attached to the 3′-end of B through a second linkage selected from the group consisting of a phosphate linkage, a phosphorothioate linkage, a phosphoramidate linkage, an amine linkage, an amide linkage, a triazole linkage, an ether linkage, and a thioether linkage.

Embodiment 307. The method of Embodiment 305 or Embodiment 306, wherein B is an mRNA molecule.

Embodiment 308. The method of Embodiment 307, wherein B encodes a second enzyme.

Embodiment 309. The method of Embodiment 308, wherein the patient in need of enzyme replacement therapy is deficient of the second enzyme.

Embodiment 310. The method of any one of Embodiments 301 to 309, wherein the patient in need of enzyme replacement therapy is no longer deficient of the first enzyme after the administering

Embodiment 311. The method of Embodiment 304 or Embodiment 309, wherein the patient in need of enzyme replacement therapy is no longer deficient of the second enzyme after the administering.

Embodiment 312. The method of any one of Embodiments 301 to 311, wherein the administering is by intravenous administration, subcutaneous administration, infusion, intramuscular administration, or oral administration.

Embodiment 313. The method of any one of Embodiments 301 to 312, wherein the pharmaceutically acceptable carrier, excipient, or diluent comprises a lipid-based carrier, a polymer-based carrier, or a nanocarrier.

Embodiment 314. The method of any one of Embodiments 301 to 313, wherein the patient in need thereof is a human.

Embodiment 315. A method of enzyme replacement therapy, the method comprising a step of administering to a patient in need of enzyme replacement therapy a pharmaceutical formulation comprising an mRNA bioconjugate of formula (II)

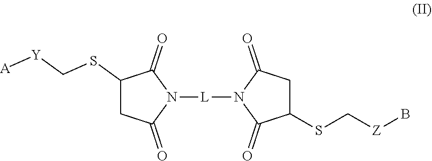

or a pharmaceutically acceptable salt thereof, and a pharmaceutically acceptable carrier, excipient, or diluent,
wherein
   A is a first mRNA molecule having a 3′-end and a 5′-end, wherein A encodes a first enzyme;
   Y is a C₁-C₂₀ alkyl, wherein any one or more —CH₂— groups of the C₁-C₂₀ alkyl is each optionally replaced independently with —C(═O)—, —CF₂—, or a heteroatomic moiety selected from the group consisting of —O—, —S—, —NH—, and —N(C₁-C₆ alkyl)-, wherein any one or more —CH₃ groups of the C₁-C₂₀ alkyl is each optionally replaced independently with —CF₃, —CF₂H, —CH₂F, or a heteroatomic moiety selected from the group consisting of —NH(C₁-C₆ alkyl), —N(C₁-C₆ alkyl)₂, —O(C₁-C₆ alkyl), and —S(C₁-C₆ alkyl), and wherein any two heteroatomic moieties are separated from one another by at least two carbon atoms;
   L is a C₂-C₅₀ alkyl, C₂-C₅₀ alkenyl, C₂-C₅₀ alkynyl, C₃-C₅ cycloalkyl, aryl, heteroaryl, C₄-C₅₀ alkyl-cycloalkyl, C₇-C₅₀ alkyl-aryl, or C₆-C₅₀ alkyl-heteroaryl, wherein any one or more —CH₂— groups of the C₂-C₅₀ alkyl is each optionally replaced independently with —C(═O)—, —CF₂—, or a heteroatomic moiety selected from the group consisting of —O—, —S—, —NH—, and —N(C₁-C₆ alkyl)-, wherein any one or more —CH₃ groups of L is each optionally replaced independently with —CF₃, —CF₂H, —CH₂F, or a heteroatomic moiety selected from the group consisting of —NH(C₁-C₆ alkyl), —N(C₁-C₆ alkyl)₂, —O(C₁-C₆ alkyl), and —S(C₁-C₆ alkyl), and wherein any two heteroatomic moieties are separated from one another by at least two carbon atoms;
   Z is a C₁-C₂₀ alkyl, wherein any one or more —CH₂— groups of the C₁-C₂₀ alkyl is each optionally replaced independently with —C(═O)—, —CF₂—, or a heteroatomic moiety selected from the group consisting of —O—, —S—, —NH—, and —N(C₁-C₆ alkyl)-, wherein any one or more —CH₃ groups of the C₁-C₂₀ alkyl is each optionally replaced independently with —CF₃, —CF₂H, —CH₂F, or a heteroatomic moiety selected from the group consisting of —NH(C₁-C₆ alkyl), —N(C₁-C₆ alkyl)₂, —O(C₁-C₆ alkyl), and —S(C₁-C₆ alkyl), and wherein any two heteroatomic moieties are separated from one another by at least two carbon atoms; and
   B is an oligonucleotide, a polypeptide, a protein, a second enzyme, a small molecule, a carbohydrate, a lipid, a polyethylene glycol (PEG) molecule, or a biopolymer,
wherein Y is covalently attached to the 3′-end of A through a first linkage, and wherein the patient in need of enzyme replacement therapy is deficient of the first enzyme.

Embodiment 316. The method of Embodiment 315, wherein the first linkage is selected from the group consisting of a phosphate linkage, a phosphorothioate linkage, a phosphoramidate linkage, an amine linkage, an amide linkage, a triazole linkage, an ether linkage, and a thioether linkage.

Embodiment 317. The method of Embodiment 315 or Embodiment 316, wherein B is a second enzyme.

Embodiment 318. The method of Embodiment 317, wherein the patient in need of enzyme replacement therapy is deficient of the second enzyme.

Embodiment 319. The method of Embodiment 315 or Embodiment 316, wherein B is an oligonucleotide having a 3′-end and a 5′-end.

Embodiment 320. The method of Embodiment 319, wherein Z is covalently attached to the 3′-end of B through a second linkage selected from the group consisting of a phosphate linkage, a phosphorothioate linkage, a phosphoramidate linkage, an amine linkage, an amide linkage, a triazole linkage, an ether linkage, and a thioether linkage.

Embodiment 321. The method of Embodiment 319 or Embodiment 320, wherein B is an mRNA molecule.

Embodiment 322. The method of Embodiment 321, wherein B encodes a second enzyme.

Embodiment 323. The method of Embodiment 322, wherein the patient in need of enzyme replacement therapy is deficient of the second enzyme.

Embodiment 324. The method of any one of Embodiments 315 to 323, wherein the patient in need of enzyme replacement therapy is no longer deficient of the first enzyme after the administering.

Embodiment 325. The method of Embodiment 318 or Embodiment 323, wherein the patient in need of enzyme replacement therapy is no longer deficient of the second enzyme after the administering

Embodiment 326. The method of any one of Embodiments 315 to 325, wherein the administering is by intravenous administration, subcutaneous administration, infusion, intramuscular administration, or oral administration.

Embodiment 327. The method of any one of Embodiments 315 to 326, wherein the pharmaceutically acceptable carrier, excipient, or diluent comprises a lipid-based carrier, a polymer-based carrier, or a nanocarrier.

Embodiment 328. The method of any one of Embodiments 315 to 327, wherein the patient in need thereof is a human.

Embodiment 329: The oligonucleotide bioconjugate of any one of Embodiments 1 to 148, further comprising one or more covalently attached carbohydrates.

Embodiment 330. The oligonucleotide bioconjugate of Embodiment 329, wherein at least one of the one or more carbohydrates is covalently attached to the 3′-end of A.

Embodiment 331. The oligonucleotide bioconjugate of Embodiment 329, wherein at least one of the one or more carbohydrates is covalently attached near the 3′-end of A.

Embodiment 332. The oligonucleotide bioconjugate of any one of Embodiments 329 to 331, wherein at least one of the one or more carbohydrates are covalently attached to the oligonucleotide bioconjugate via a sulfide linker or a peptide linker.

Embodiment 333. The oligonucleotide bioconjugate of any one of Embodiments 329 to 332, wherein the one or more carbohydrates is selected from the group consisting of GalNAc, mannose-6-phosphate, mannose, high-branched mannose, sialic acid, and galactose.

Embodiment 334. The method of any one of Embodiments 205 to 328, wherein the mRNA bioconjugate further comprises one or more covalently attached carbohydrates.

Embodiment 335. The method of Embodiment 334, wherein at least one of the one or more carbohydrates is covalently attached to the 3′-end of A.

Embodiment 336. The method of Embodiment 334, wherein at least one of the one or more carbohydrates is covalently attached near the 3′-end of A.

Embodiment 337. The method of any one of Embodiments 334 to 336, wherein at least one of the one or more carbohydrates are covalently attached to the oligonucleotide bioconjugate via a sulfide linker or a peptide linker.

Embodiment 338. The method of any one of Embodiments 334 to 337, wherein the one or more covalently attached carbohydrates is selected from the group consisting of GalNAc, mannose-6-phosphate, mannose, high-branched mannose, sialic acid, and galactose.

Embodiment 339. The method of any one of Embodiments 165 to 172, further comprising a step of covalently attaching one or more carbohydrates to the oligonucleotide bioconjugate of formula (I).

Embodiment 340. The method of Embodiment 339, wherein at least one of the one or more carbohydrates is covalently attached to the 3′-end of A.

Embodiment 341. The method of Embodiment 339, wherein at least one of the one or more carbohydrates is covalently attached near the 3′-end of A.

Embodiment 342. The method of any one of Embodiments 339 to 341, wherein at least one of the one or more carbohydrates are covalently attached to the oligonucleotide bioconjugate via a sulfide linker or a peptide linker.

Embodiment 343. The method of any one of Embodiments 339 to 342, wherein the one or more carbohydrates is selected from the group consisting of GalNAc, mannose-6-phosphate, mannose, high-branched mannose, sialic acid, and galactose.

Embodiment 344. The method of any one of Embodiments 173 to 180, further comprising a step of covalently attaching one or more carbohydrates to the oligonucleotide bioconjugate of formula (II).

Embodiment 345. The method of Embodiment 344, wherein at least one of the one or more carbohydrates is covalently attached to the 3′-end of A.

Embodiment 346. The method of Embodiment 344, wherein at least one of the one or more carbohydrates is covalently attached near the 3′-end of A.

Embodiment 347. The method of any one of Embodiments 344 to 346, wherein at least one of the one or more carbohydrates are covalently attached to the oligonucleotide bioconjugate via a sulfide linker or a peptide linker.

Embodiment 348. The method of any one of Embodiments 344 to 347, wherein the one or more covalently attached carbohydrates is selected from the group consisting of GalNAc, mannose-6-phosphate, mannose, high-branched mannose, sialic acid, and galactose.

Embodiment 349. The method of any one of Embodiments 181 to 188, further comprising a step of covalently attaching one or more carbohydrates to the oligonucleotide bioconjugate of formula (III).

Embodiment 350. The method of Embodiment 349, wherein at least one of the one or more carbohydrates is covalently attached to the 3′-end of A.

Embodiment 351. The method of Embodiment 349, wherein at least one of the one or more carbohydrates is covalently attached near the 3′-end of A.

Embodiment 352. The method of any one of Embodiments 349 to 351, wherein at least one of the one or more carbohydrates are covalently attached to the oligonucleotide bioconjugate via a sulfide linker or a peptide linker.

Embodiment 353. The method of any one of Embodiments 349 to 352, wherein the one or more covalently attached carbohydrates is selected from the group consisting of GalNAc, mannose-6-phosphate, mannose, high-branched mannose, sialic acid, and galactose.

Embodiment 354. The method of any one of Embodiments 189 to 196, further comprising a step of covalently attaching one or more carbohydrates to the oligonucleotide bioconjugate of formula (IV).

Embodiment 355. The method of Embodiment 354, wherein at least one of the one or more carbohydrates is covalently attached to the 3′-end of A.

Embodiment 356. The method of Embodiment 354, wherein at least one of the one or more carbohydrates is covalently attached near the 3′-end of A.

Embodiment 357. The method of any one of Embodiments 354 to 356, wherein at least one of the one or more carbohydrates are covalently attached to the oligonucleotide bioconjugate via a sulfide linker or a peptide linker.

Embodiment 358. The method of any one of Embodiments 354 to 357, wherein the one or more covalently attached carbohydrates is selected from the group consisting of GalNAc, mannose-6-phosphate, mannose, high-branched mannose, sialic acid, and galactose.

Embodiment 359. The method of any one of Embodiments 197 to 204, further comprising a step of covalently attaching one or more carbohydrates to the oligonucleotide bioconjugate of formula (V).

Embodiment 360. The method of Embodiment 359, wherein at least one of the one or more carbohydrates is covalently attached to the 3′-end of A.

Embodiment 361. The method of Embodiment 359, wherein at least one of the one or more carbohydrates is covalently attached near the 3′-end of A.

Embodiment 362. The method of any one of Embodiments 359 to 361, wherein at least one of the one or more carbohydrates are covalently attached to the oligonucleotide bioconjugate via a sulfide linker or a peptide linker.

Embodiment 363. The method of any one of Embodiments 359 to 362, wherein the one or more covalently attached carbohydrates is selected from the group consisting of GalNAc, mannose-6-phosphate, mannose, high-branched mannose, sialic acid, and galactose.

Embodiment 364. The oligonucleotide bioconjugate of Embodiment 16 or Embodiment 57, wherein B is a cell penetrating polypeptide.

Embodiment 365. The oligonucleotide bioconjugate of any one of Embodiments 16, 57, and 364, wherein B is selected from the group consisting of a polycationic cell penetrating polypeptide, an amphipathic cell penetrating polypeptide, and a hydrophobic cell penetrating polypeptide.

Embodiment 366. The oligonucleotide bioconjugate of any one of Embodiments 16, 57, 364, and 365, wherein B has an amino acid sequence selected from the group consisting of SEQ ID NOs: 1 to 71.

Embodiment 367: The method of any one of Embodiments 232, 249, 263, 271, 285, and 295, wherein B is a cell penetrating polypeptide.

Embodiment 368. The method of any one of Embodiments 232, 249, 263, 271, 285, 295, and 367, wherein B is selected from the group consisting of a polycationic cell penetrating polypeptide, an amphipathic cell penetrating polypeptide, and a hydrophobic cell penetrating polypeptide.

Embodiment 369. The method of any one of Embodiments 232, 249, 263, 271, 285, 295, 367, and 368, wherein B has an amino acid sequence selected from the group consisting of SEQ ID NOs: 1 to 71.

Embodiment 370. The oligonucleotide bioconjugate of any one of Embodiments 1 to 148, wherein A encodes a therapeutic polypeptide.

Embodiment 371. The oligonucleotide bioconjugate of Embodiment 370, wherein the therapeutic polypeptide has anti-cancer activity.

Embodiment 372. The oligonucleotide bioconjugate of Embodiment 370, wherein the therapeutic polypeptide has anti-obesity activity.

Embodiment 373. The oligonucleotide bioconjugate of Embodiment 370, wherein A encodes an amino acid sequence selected from the group consisting of SEQ ID NOs: 1 to 71.

Embodiment 374. The oligonucleotide bioconjugate of any one of Embodiments 1 to 148, wherein A has a nucleotide sequence selected from the group consisting of SEQ ID NOs: 72 to 74.

Embodiment 375. The method of any one of Embodiments 205 to 328, wherein A encodes a therapeutic polypeptide.

Embodiment 376. The method of Embodiment 375, wherein the therapeutic polypeptide has anti-cancer activity.

Embodiment 377. The method of Embodiment 375, wherein the therapeutic polypeptide has anti-obesity activity.

Embodiment 378. The method of Embodiment 375, wherein A encodes an amino acid sequence selected from the group consisting of SEQ ID NOs: 1 to 71.

Embodiment 379. The method of any one of Embodiments 205 to 328, wherein A has a nucleotide sequence selected from the group consisting of SEQ ID NOs: 72 to 74.

Embodiment 380. The method of any one of Embodiments 165 to 204, wherein A encodes a therapeutic polypeptide.

Embodiment 381. The method of Embodiment 380, wherein the therapeutic polypeptide has anti-cancer activity.

Embodiment 382. The method of Embodiment 380, wherein the therapeutic polypeptide has anti-obesity activity.

Embodiment 383. The method of Embodiment 380, wherein A encodes an amino acid sequence selected from the group consisting of SEQ ID NOs: 1 to 71.

Embodiment 384. The method of any one of Embodiments 165 to 204, wherein A has a nucleotide sequence selected from the group consisting of SEQ ID NOs: 72 to 74.

Embodiment 385. The oligonucleotide bioconjugate of any one of Embodiments 8 to 14, and 49 to 55, wherein B encodes a therapeutic polypeptide.

Embodiment 386. The oligonucleotide bioconjugate of Embodiment 385, wherein the therapeutic polypeptide has anti-cancer activity.

Embodiment 387. The oligonucleotide bioconjugate of Embodiment 385, wherein the therapeutic polypeptide has anti-obesity activity.

Embodiment 388. The oligonucleotide bioconjugate of Embodiment 385, wherein B encodes an amino acid sequence selected from the group consisting of SEQ ID NOs: 1 to 71.

Embodiment 389. The oligonucleotide bioconjugate of any one of Embodiments 8 to 14, and 49 to 55, wherein B has a nucleotide sequence selected from the group consisting of SEQ ID NOs: 72 to 74.

Embodiment 390. The method of any one of Embodiments 205 to 224, 228 to 231, 245 to 248, 261, 262, 272, 273, 283, 284, 293, 294, 305 to 309, and 319 to 323, wherein B encodes a therapeutic polypeptide.

Embodiment 391. The method of Embodiment 390, wherein the therapeutic polypeptide has anti-cancer activity.

Embodiment 392. The method of Embodiment 390, wherein the therapeutic polypeptide has anti-obesity activity.

Embodiment 393. The method of Embodiment 390, wherein B encodes an amino acid sequence selected from the group consisting of SEQ ID NOs: 1 to 71.

Embodiment 394. The method of any one of Embodiments 129 to 148, 152 to 155, 169 to 172, 185, 186, 196, 197, 207, 208, 217, 218, 229 to 233, and 243 to 247, wherein B has a nucleotide sequence selected from the group consisting of SEQ ID NOs: 72 to 74.

Embodiment 395. The method of any one of Embodiments 169 to 172, 177 to 180, 185 to 188, 193 to 196, and 201 to 204, wherein B encodes a therapeutic polypeptide.

Embodiment 396. The method of Embodiment 395, wherein the therapeutic polypeptide has anti-cancer activity.

Embodiment 397. The method of Embodiment 395, wherein the therapeutic polypeptide has anti-obesity activity.

Embodiment 398. The method of Embodiment 395, wherein B encodes an amino acid sequence selected from the group consisting of SEQ ID NOs: 1 to 71.

Embodiment 399. The method of any one of Embodiments 169 to 172, 177 to 180, 185 to 188, 193 to 196, and 201 to 204, wherein B has a nucleotide sequence selected from the group consisting of SEQ ID NOs: 72 to 74.

### H. Examples

### Example 1: Preparation of Oligonucleotide Bioconjugates and Characterization of Such

All reverse phase high performance liquid chromatography (RP-HPLC) is performed on an Agilent 1290 Infinity II UPLC Stack with autosampler attached to a Agilent 6470 mass spectrometer.

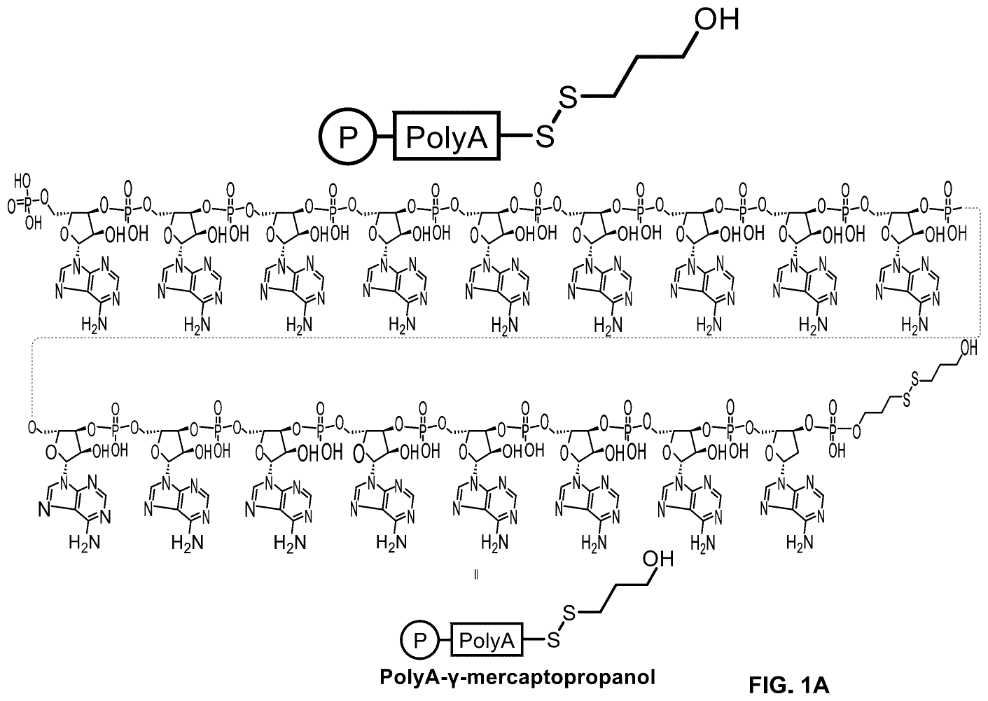

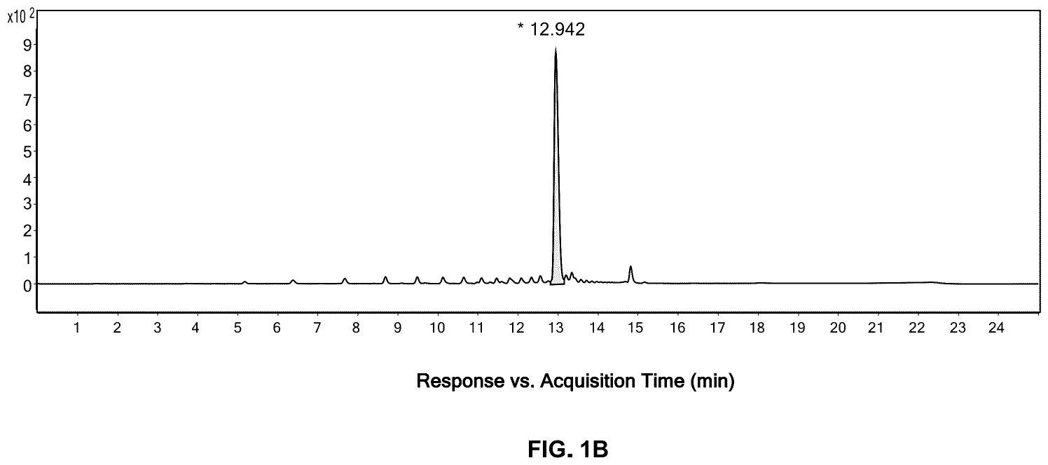

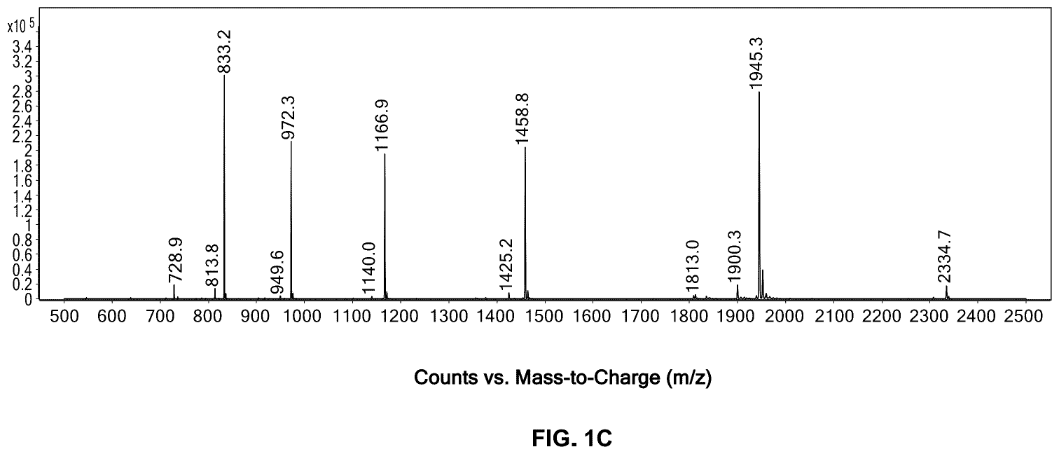

PolyA-γ-mercaptopropanol is bought from Integrated DNA Technologies (FIG. 1A). The purity of the sample is confirmed by RP-HPLC (FIG. 1B) and mass spectrometry (MS) (FIG. 1C) with parameters as shown in Table 1. The observed mass of 1945.3 Dalton for the sample corresponded well to the calculated mass of C₁₇₆H₂₁₆N₈₅O₁₀₆P₁₈S₂³⁻ at 1945.6 Daltons.

TABLE 1
RP-HPLC and MS parameters for determining
purity and identity of precursor.

| | |
|---|---|
| Column | DNAPac RP column, 2.1 × 100 mm, 4 μm |
| Column Temperature | 85° C. |
| Column flow rate | 0.4 mL/min |
| Solvent A | Water:Triethylamine:HFIP:MeOH v/v/v/v |
| | 477.9:1.05:21.06:0 |
| Solvent B | Water:Triethylamine:HFIP:MeOH v/v/v/v |
| | 227.9:1.05:21.06:250 |
| Gradient | 0.0-1.0 min 0% B, |
| | 1.0-10.0 min 0-36% B, |
| | 10.0-18.5 min 36-70% B, |
| | 18.5-20.5 min 70-100% B, |
| | 20.5-21.0 min 100-0% B, |
| | 21.0-25.0 min 0% B |
| Mass Spectrometry | Negative electrospray ionization (ESI) |
| Mass Range | 500-2500 m/z |
| Temperature of drying gas | 350° C. |
| Flow rate of drying gas | 13 L/min |
| Pressure of nebulizer gas | 55 psi |
| Sheath gas temperature | 400° C. |
| Sheath gas flow | 12 L/min |
| Capillary positive voltage | 4500 V |
| Capillary negative voltage | 3500 V |
| Nozzle positive voltage | 0 V |
| Nozzle negative voltage | 2000 V |

### Conversion to PolyA-SH

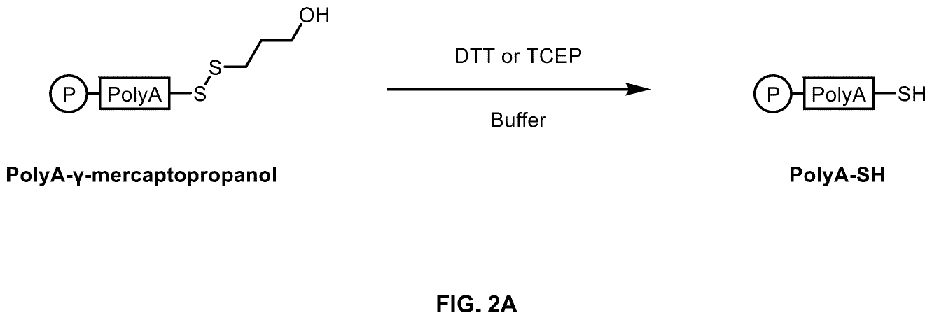

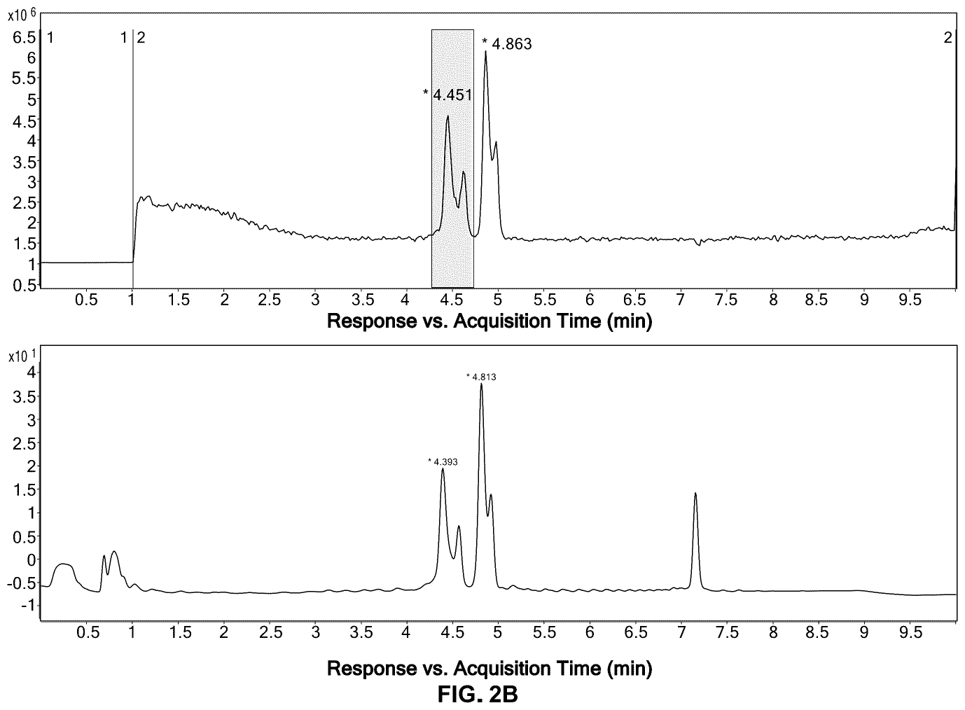

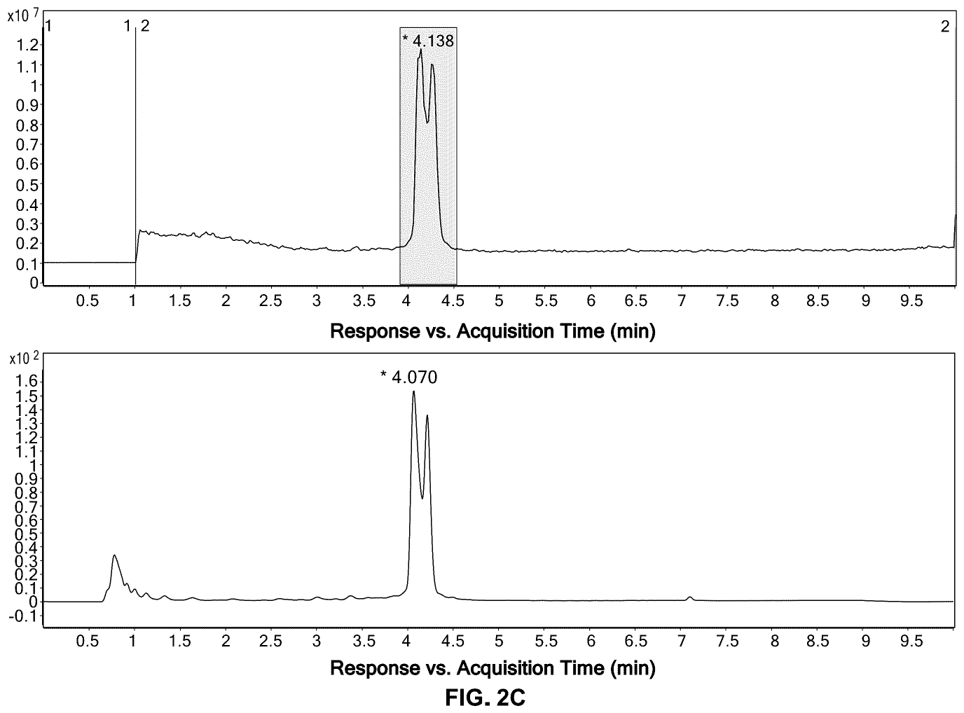

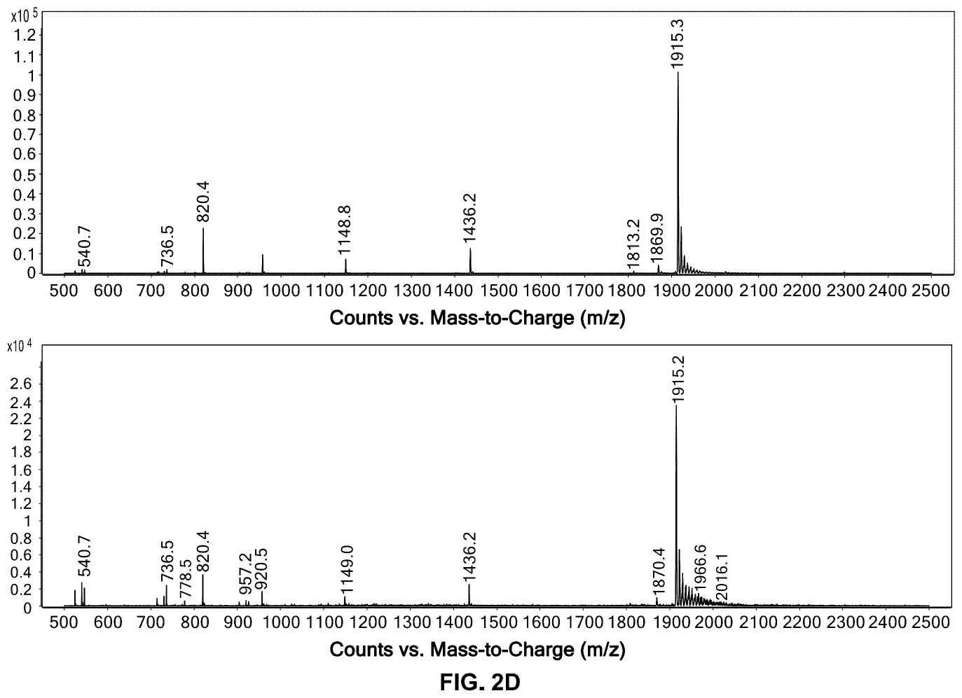

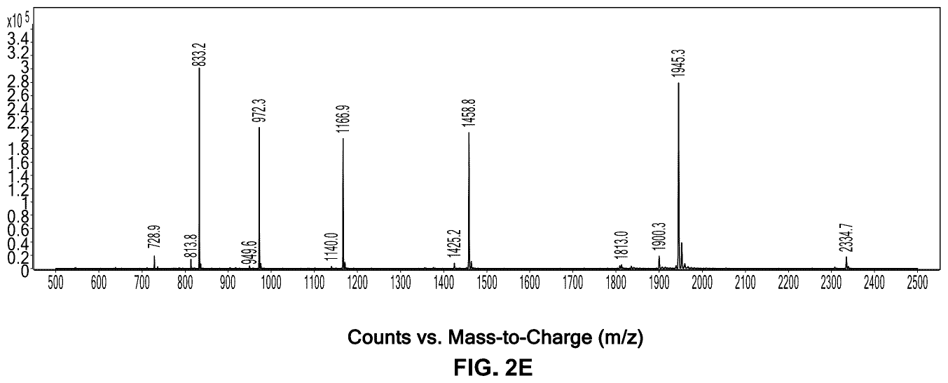

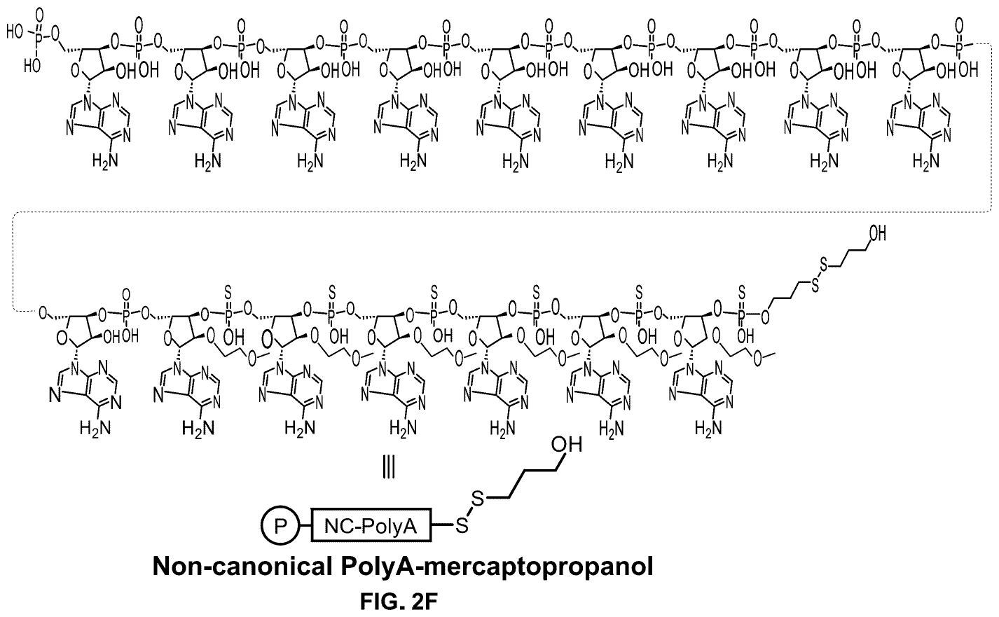

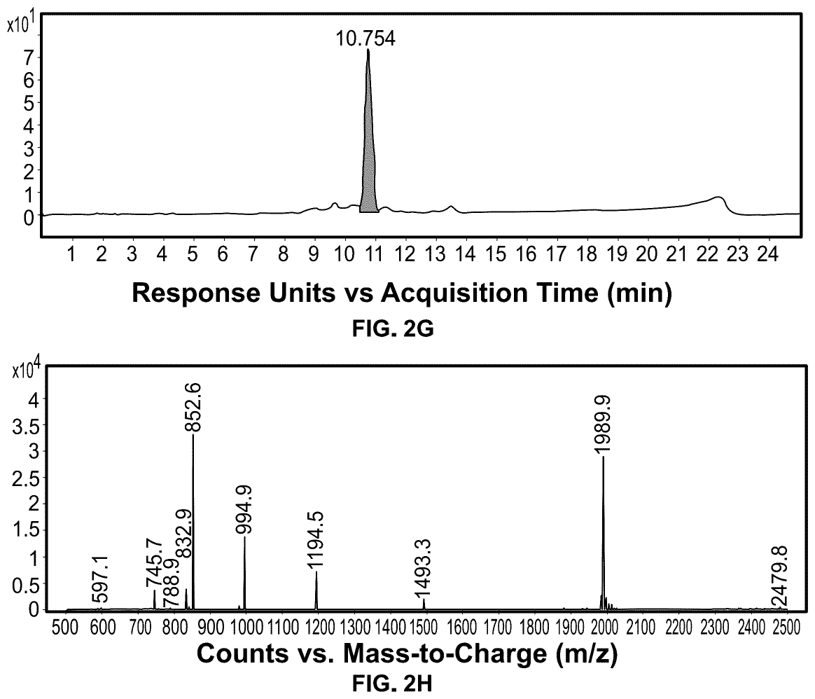

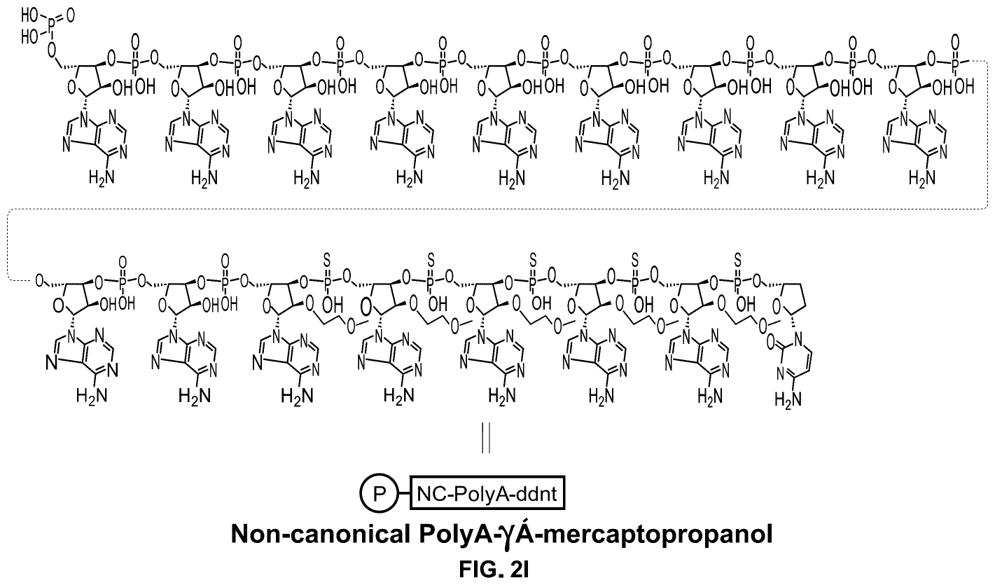

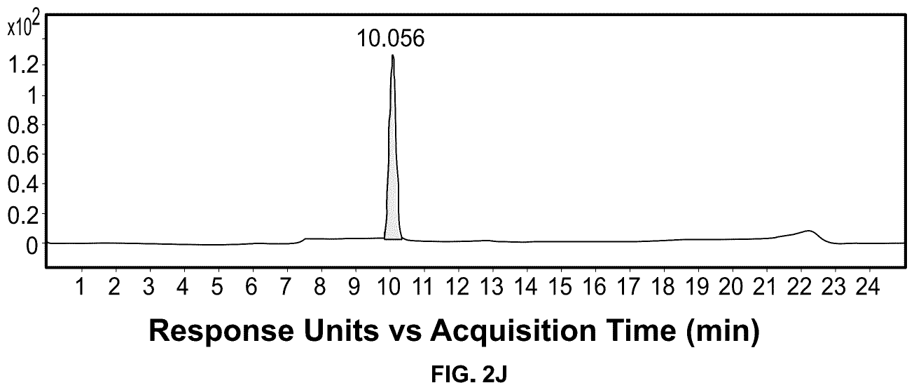

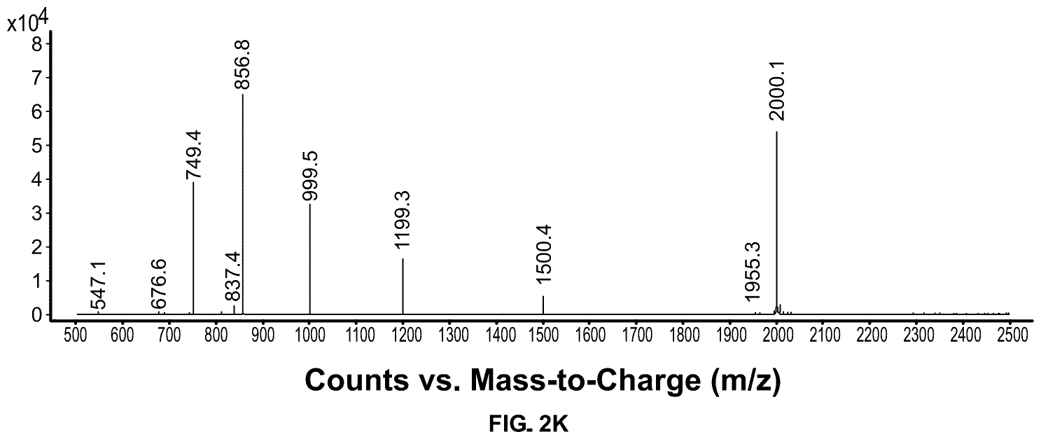

PolyA-γ-mercaptopropanol (SEQ ID NO: 75) is converted to PolyA-SH (SEQ ID NO: 76) by reduction (FIG. 2A). Briefly, PolyA-γ-mercaptopropanol (20 μL, 500 μM in water) is added to a solution of dithiothreitol (DTT) (5 μL, 200 mM in 200 mM Tris, 1.5 M NaCl pH 7.6 buffer) and incubated at room temperature. The reaction is monitored by RP-HPLC and MS using parameters as shown in Table 2. Two separate runs of the reaction show similar results. As shown by the total ion count chromatogram in FIG. 2B, PolyA-SH appears as two peaks (highlighted) on the left (+4.451 min in Run 1 (top) and +4.393 min in Run 2 (bottom)) while PolyA-γ-mercaptopropanol also appears as two peaks on the right (+4.863 min in Run 1 (top) and +4.813 min in Run 2 (bottom)). In the event of the reaction stalling, additional 3 μL of DTT solution is added followed by the addition of 3 μL of 200 mM Tris, 1.5 M NaCl, pH 7.6 buffer. Upon completion of the reaction as observed by RP-HPLC UV chromatogram (Run 1: FIG. 2C (top) and Run 2: FIG. 2C (bottom)), the entirety of the mixture is buffer exchanged/desalted using a 7 kDa MWCO Zeba 0.5 mL desalting column according to the manufacturer's protocols. The resultant solution has a concentration of 1396.2 ng/μL as measured by its absorbance at 260 nm on a nanodrop instrument with a total recovery volume of ˜50 μL. The extracted mass spectrum of the purified PolyA-SH (the highlighted peak in FIG. 2B) is shown in FIG. 2D, where the observed mass of 1915.2 Da (Run 1) and 1915.3 Da (Run 2) correspond closely the calculated mass of 1915.6 Da. Mass spectrometry of the purified PolyA-γ-mercaptopropanol is shown in FIG. 2E, with an observed mass of 1945.3 Da.

TABLE 2
RP-HPLC and MS parameters for monitoring
conversion to PolyA-SH.

| | |
|---|---|
| Column | DNAPac RP column, 2.1 × 100 mm, 4 μm |
| Column Temperature | 85° C. |
| Column flow rate | 0.2 mL/min |
| Solvent A | Water:Triethylamine:HFIP:MeOH v/v/v/v |
| | 427.9:1.05:21.06:50 |
| Solvent B | Water:Triethylamine:HFIP:MeOH v/v/v/v |
| | 227.9:1.05:21.06:250 |
| Gradient | 0.0-0.5 min 0-30% B, |
| | 0.5-7.0 min 30-100% B, |
| | 7.0-7.5 min 100% B, |
| | 7.5-8.0 min 100-0% B, |
| | 8.0-10.0 min 0% B |
| Mass Spectrometry | Negative electrospray ionization (ESI) |
| Mass Range | 500-2500 m/z |
| Temperature of drying gas | 350° C. |
| Flow rate of drying gas | 13 L/min |
| Pressure of nebulizer gas | 55 psi |
| Sheath gas temperature | 400° C. |
| Sheath gas flow | 12 L/min |
| Capillary positive voltage | 4500 V |
| Capillary negative voltage | 3500 V |
| Nozzle positive voltage | 0 V |
| Nozzle negative voltage | 2000 V |

Non-canonical PolyA-γ-mercaptopropanol

Non-canonical PolyA-γ-mercaptopropanol (SEQ ID NO: 77) (FIG. 2F) is obtained from IDT and is used as received. The non-canonical PolyA-γ-mercaptopropanol is analyzed by RP-HPLC (FIG. 2G) and using the parameters listed in Table 6. By mass spectrogram (FIG. 2H), the observed mass of 1989.9 Da corresponds closely to the calculated mass of 1989.6 Da.

### Non-Canonical PolyA-dideoxynucleotide

Non-canonical PolyA-dideoxynucleotide (SEQ ID NO: 78) (FIG. 2I) is obtained from IDT and is used as received. The non-canonical PolyA-dideoxynucleotide is analyzed by RP-HPLC (FIG. 2J) and using the parameters listed in Table 6. By mass spectrogram (FIG. 2K), the observed mass of 1999.5 Da corresponds closely to the calculated mass of 1999.0 Da.

### Synthesis of PolyA-S-Maleimide-SulfoCy5

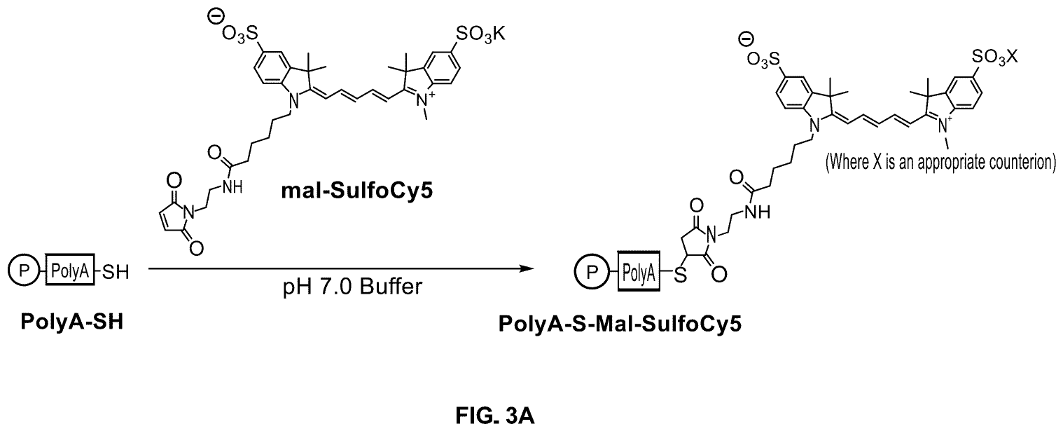

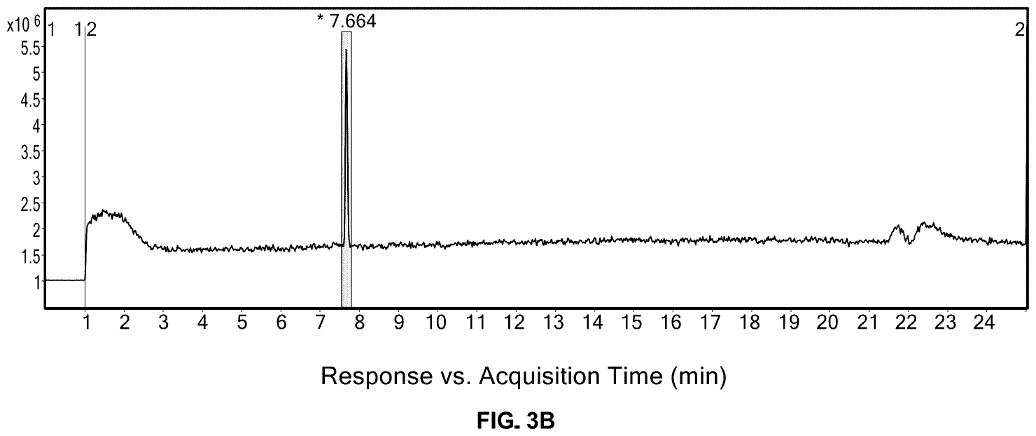

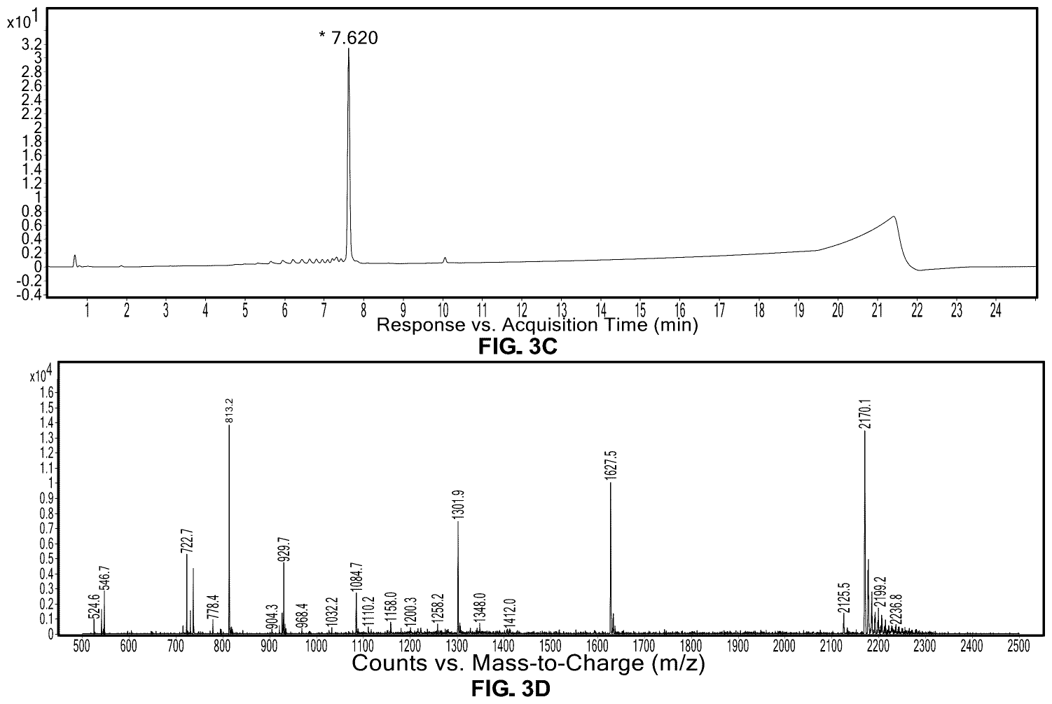

PolyA-S-Maleimide-SulfoCy5 (SEQ ID NO: 79) is synthesized from purified PolyA-SH derived as described herein (FIG. 3A). Briefly, mal-SulfoCy5 (2.5 μL, 50.0 mM solution in DMSO) is added to PolyA-SH (146 μL, 1.904 mg/mL as measured by A260). The solution is mixed by pipetting up and down 20 times and the mixture is allowed to incubate at room temperature. The reaction is monitored by RP-HPLC and MS using parameters as shown in Table 3. The peak representing PolyA-S-Maleimide-SulfoCy5 in a total ion count chromatogram is shown in FIG. 3B. The peak representing PolyA-S-Maleimide-SulfoCy5 as seen in the UV chromatogram is shown in FIG. 3C. The extracted mass spectrum of the peak highlighted in FIG. 3B is shown in FIG. 3D, with the observed mass of 2170.1 Da for PolyA-S-Maleimide-SulfoCy5 (C₂₁₁H₂₅₄N₈₉O₁₁₄P₁₈S₃³⁻) corresponding well to a calculated mass of 2170.4 Da. Upon complete consumption of the starting material as observed by RP-HPLC, the entirety of the mixture is buffer exchanged/desalted using a 7 kDa MWCO Zeba 0.5 mL desalting column according to the manufacturer's protocols. The resulting solution of PolyA-S-Maleimide-SulfoCy5 (SEQ ID NO: 79; 179 μL, 1272.2 ng/μL) is used in subsequent experiments without further purification unless otherwise specified.

TABLE 3
RP-HPLC and MS parameters for monitoring
synthesis of PolyA-S-Maleimide-SulfoCy5.

| | |
|---|---|
| Column | DNAPac RP column, 2.1 × 100 mm, 4 μm |
| Column Temperature | 85° C. |
| Column flow rate | 0.4 mL/min |
| Solvent A | Water:Triethylamine:HFIP:MeOH v/v/v/v |
| | 427.9:1.05:21.06:50 |
| Solvent B | Water:Triethylamine:HFIP:MeOH v/v/v/v |
| | 227.9:1.05:21.06:250 |
| Gradient | 0.0-1.0 min 0% B, |
| | 1.0-10.0 min 0-36% B, |
| | 10.0-18.5 min 36-70% B, |
| | 18.5-20.5 min 70-100% B, |
| | 20.5-21.0 min 100-0% B, |
| | 21.0-25.0 min 0% B |
| Mass Spectrometry | Negative electrospray ionization (ESI) |
| Mass Range | 500-2500 m/z |
| Temperature of drying gas | 350° C. |
| Flow rate of drying gas | 13 L/min |
| Pressure of nebulizer gas | 55 psi |
| Sheath gas temperature | 400° C. |
| Sheath gas flow | 12 L/min |
| Capillary positive voltage | 4500 V |
| Capillary negative voltage | 3500 V |
| Nozzle positive voltage | 0 V |
| Nozzle negative voltage | 2000 V |

### Synthesis of PolyA-S-Maleimide-PEG19-Maleimide

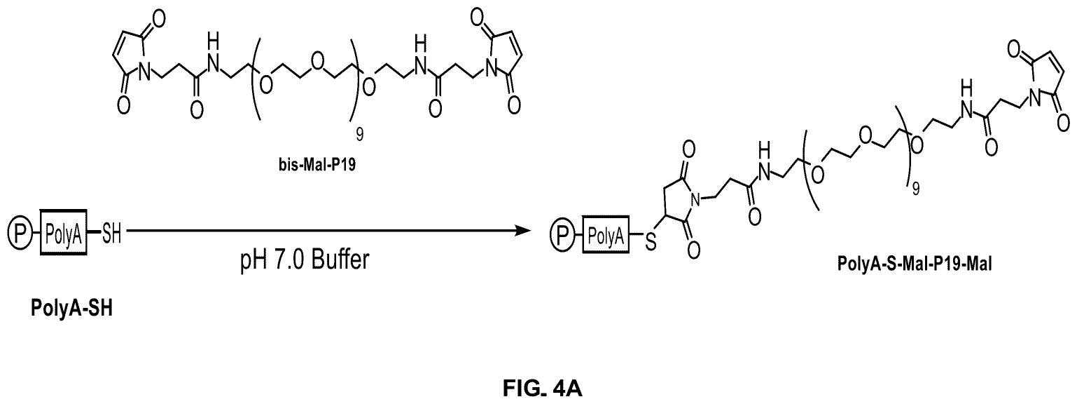

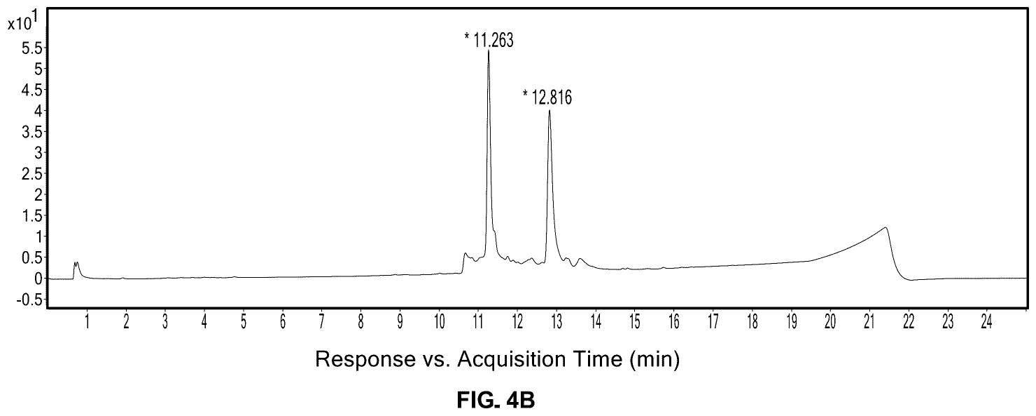

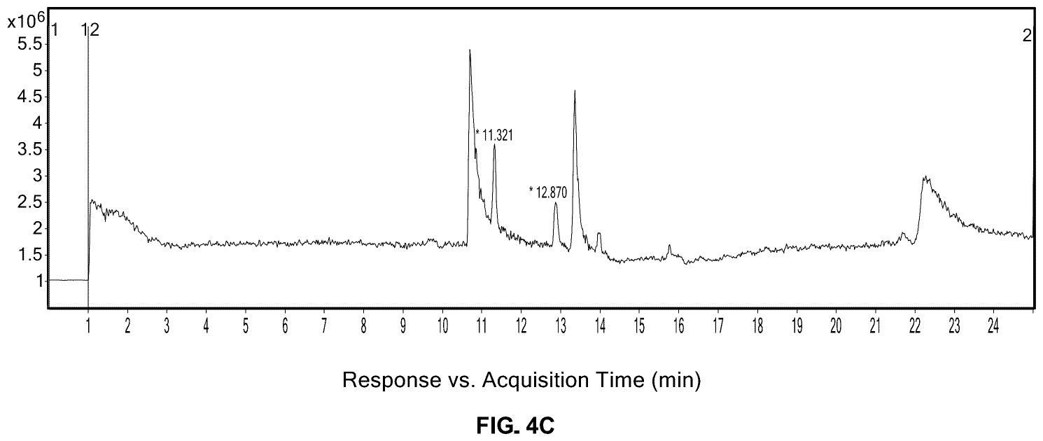

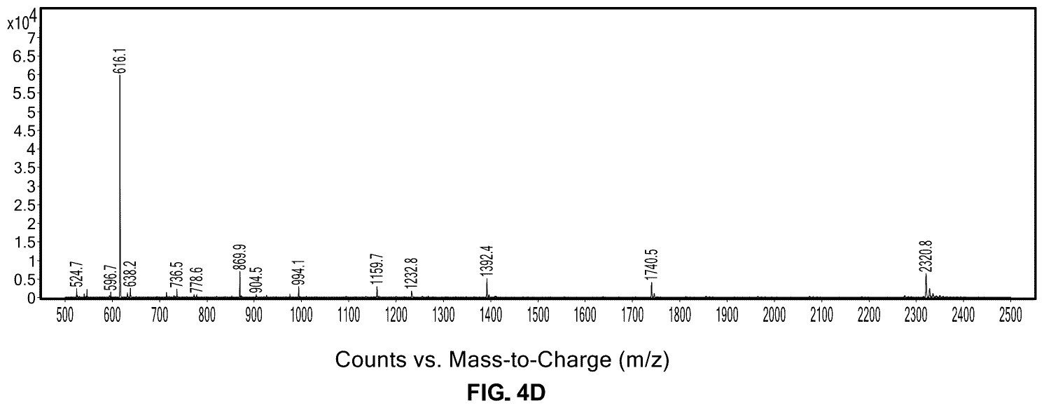

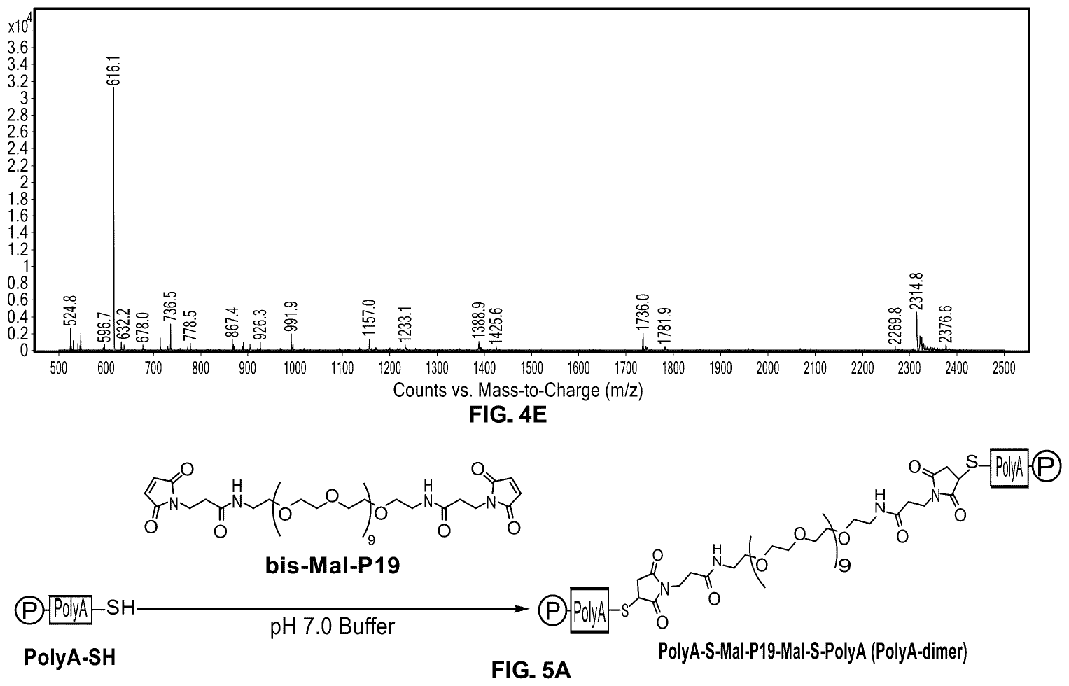

PolyA-S-Maleimide-PEG19-Maleimide (SEQ ID NO: 80) is synthesized from purified PolyA-SH derived as described herein (FIG. 4A). Briefly, PolyA-SH (5 μL, 1396.2 ng/μL in 5 μL, 20 mM Tris, 150 mM NaCl, pH 7.0), Tris 1× Buffer (5 μL, 20 mM Tris, 150 mM NaCl, pH 7.0), and bis-Maleimide-PEG19 (1.2 μL, 10.0 mM solution in DMSO) are added sequentially to a 0.5 mL Eppendorf tube. The reaction is mixed by pipetting up and down and allowed to incubate at room temperature. The reaction is monitored by UV RP-HPLC and mass spectrometry using parameters as shown in Table 3. The peak as measured by UV absorbance representing PolyA-S-Maleimide-PEG19-Maleimide is shown in FIG. 4B. The total ion count chromatogram of PolyA-S-Maleimide-PEG19-Maleimide is shown in FIG. 4C. The extracted mass spectrum peak at 11.321 min representing PolyA-S-Maleimide-PEG19-Maleimide (hydrolyzed) is shown in FIG. 4D, with an observed mass of 2320.8 Da, corresponding well to a calculated mass of 2321.2 Da. The extracted mass spectrum peak at 12.870 min representing PolyA-S-Maleimide-PEG19-Maleimide (unhydrolyzed) is shown in FIG. 4E, with an observed mass of 2314.8 Da, corresponding well to a calculated mass of 2315.2 Da. After 1 hour, the entirety of the mixture is desalted/buffer exchanged using a Zeba Spin Micro column according to the manufacturers protocol. The resulting solution containing PolyA-S-Maleimide-PEG19-Maleimide is estimated by A260 to be at a concentration of 343.3 ng/μL with a total volume of ˜14 μL.

Synthesis of PolyA-S-Maleimide-PEG19-Maleimide-S-PolyA (also referred to as “PolyA-dimer”)

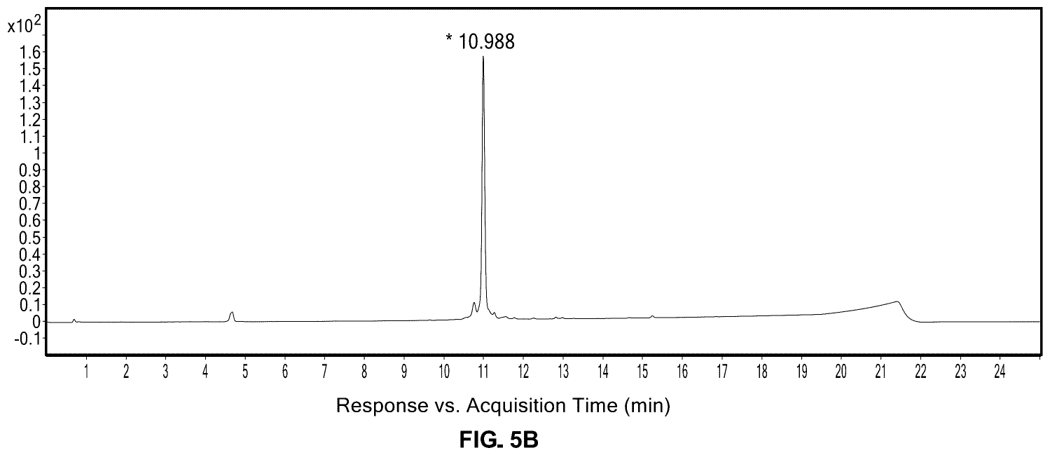

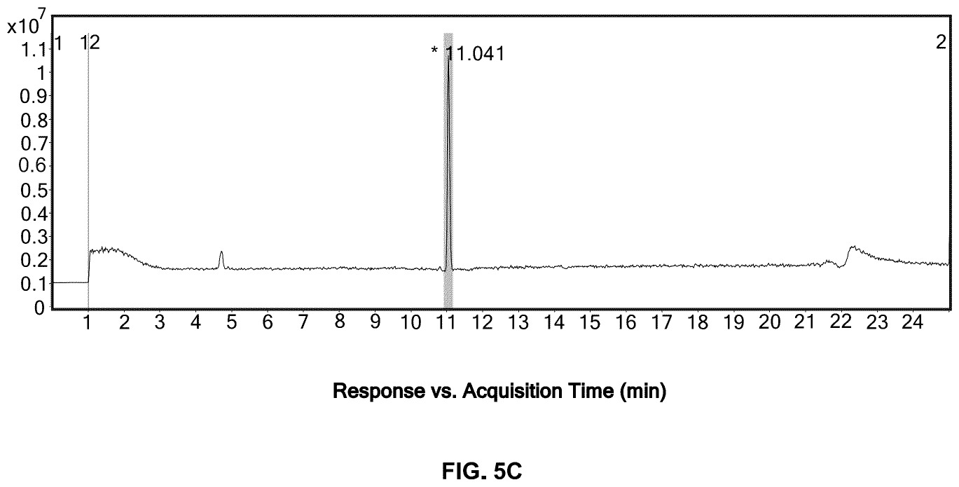

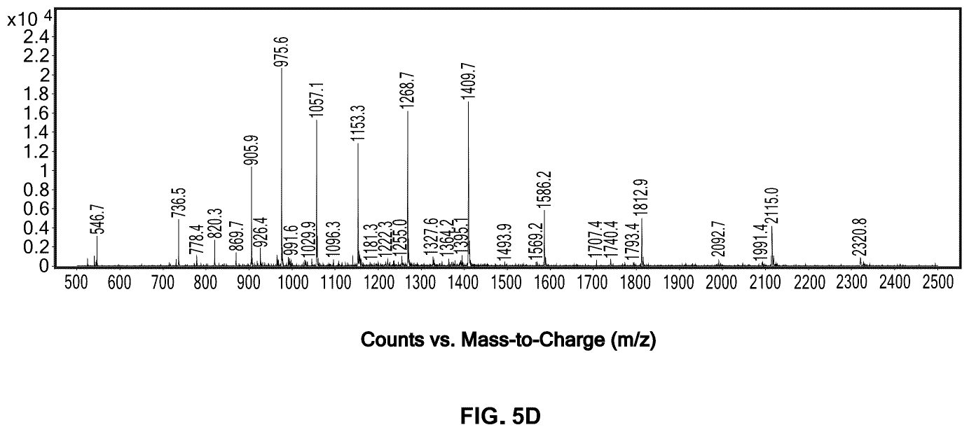

PolyA-S-Maleimide-PEG19-Maleimide-S-PolyA (SEQ ID NO: 81) is synthesized from the purified PolyA-S-Maleimide-PEG19-Maleimide derived as described above (FIG. 5A). Briefly, PolyA-SH (2.95 μL, 1396.2 ng/μL) is added to the PolyA-S-Maleimide-PEG19-Maleimide derived previously (14 μL, 343.3 ng/μL) and mixed by pipetting up and down 20 times, and then incubated for 2-5 min. The reaction is then monitored by RP-HPLC and MS using parameters as shown in Table 3. This process of adding PolyA-SH (in 1.0 to 5.90 μL aliquots), mixing, incubating for 2-5 min, and then monitoring by RP-HPLC is repeated until consumption of the starting material, as observed by both retention time and by analysis of the resulting mass spectrum of major peaks. The final solution has a total volume of ˜40-50 μL with a measured A260 concentration of 1112.7 ng/μL. The peak representing UV absorbance representing PolyA-S-Maleimide-PEG19-Maleimide-S-PolyA is shown in FIG. 5B. The total ion count chromatogram of PolyA-S-Maleimide-PEG19-Maleimide-S-PolyA is shown in FIG. 5C. The extracted mass spectrum peak highlighted in grey in FIG. 5C representing PolyA-S-Maleimide-PEG19-Maleimide-S-PolyA is shown in FIG. 5D, with an observed mass of 2115.0 Da, corresponding well to a calculated mass of 2115.4 Da.

Enzymatic Ligation with T4 RNA Ligase I: General Scheme

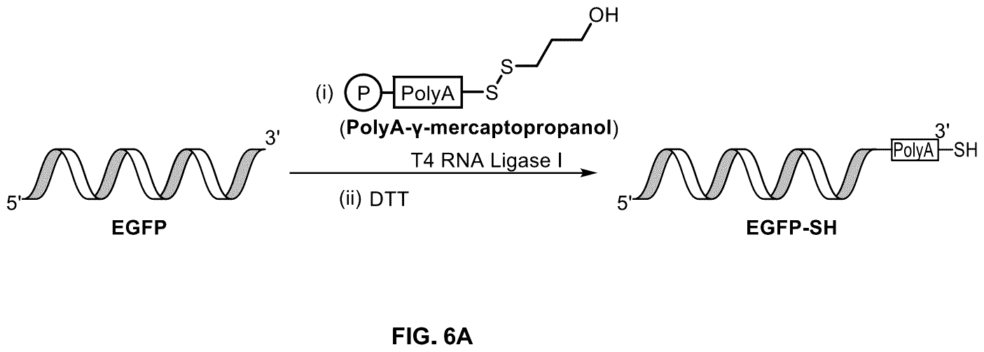

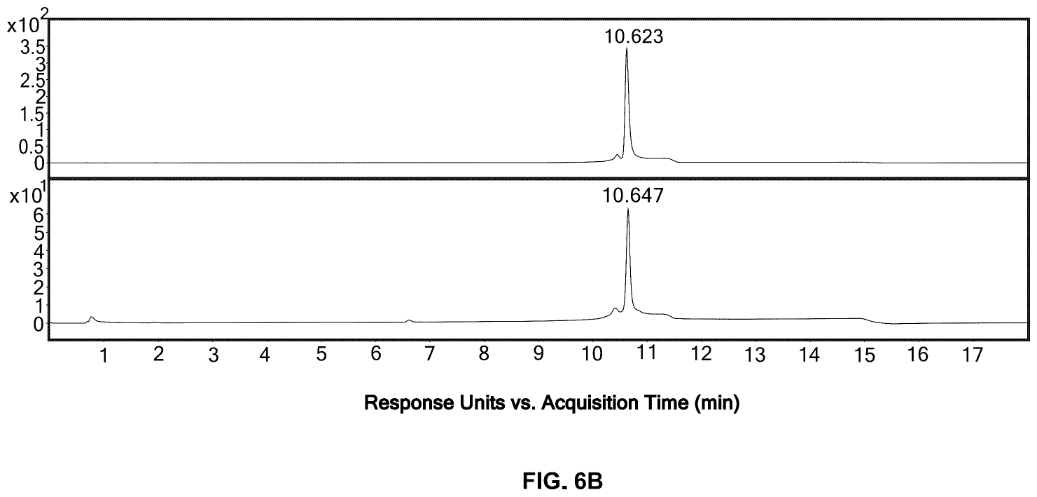

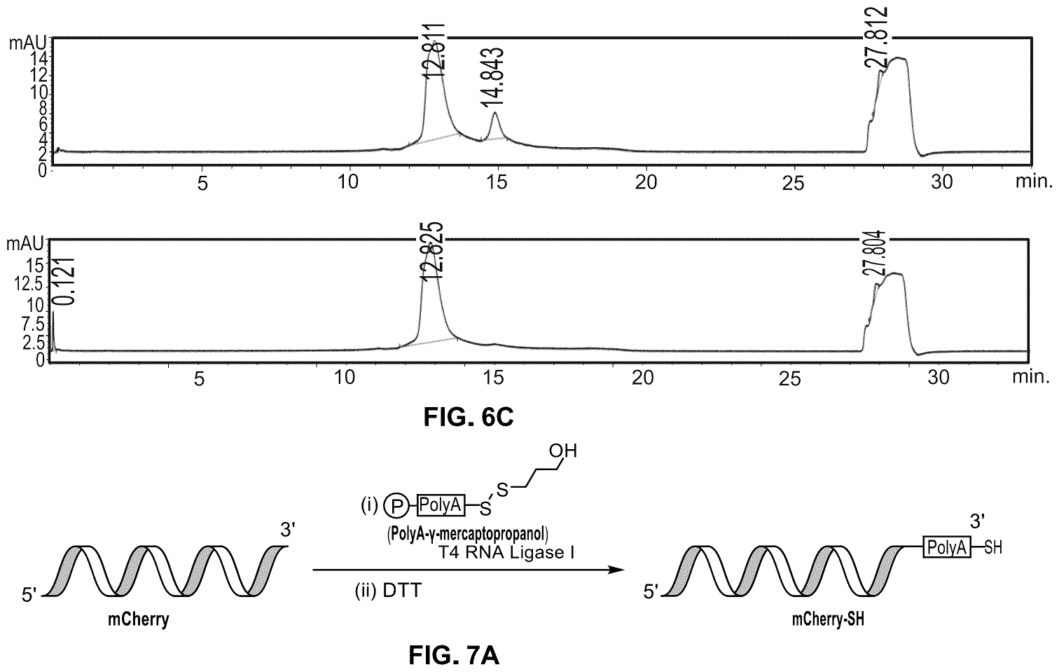

T4 RNA Ligase I 5000 units kit is procured from New England Biolabs, Inc. Target mRNA is ligated to PolyA-γ-mercaptopropanol using T4 RNA ligase I to form Target mRNA-PolyA-SH (FIGS. 6A, 7A, and 8A). For all the different Target mRNAs, ligation is performed by adding the following, in order, to a 0.5 mL Eppendorf tube:
i) Target mRNA (see Table 5)
ii) PolyA-γ-mercaptopropanol (6.0 μL, 500 μM solution in water, 3.0 nmol)
iii) Protector RNase Inhibitor (1.53 μL, used as received and obtained from Millipore-Sigma, Cat. No. 3335399001)
iv) 1Ox Ligation buffer (30.6 μL)
v) ATP (3.1 μL, provided as a 100 mM stock solution, 310 nmol)
vi) Water (adjusted to bring total reaction volume to 280 μL, see Table 5)
vii) 50% PEG-8000 (122.4 μL)
viii) T4 RNA Ligase I (30.6 μL, 30,000 units/mL)

The reaction is mixed by pipetting up and down after each addition. The mixture is incubated for 3-8 hours at room temperature or overnight at 5° C. After this time, in instances where PolyA-γ-mercaptopropanol is used, a solution of DTT (30.1 μL, 200 mM solution in Tris 10× buffer, pH 7.6) is added and the mixture allowed to incubate for 1 hour. The entire mixture is purified using any one of the following three purification methods: (i) AMPure XP Beads used according to the manufacturer's protocol (Purification Method 1); (ii) buffer exchange in Tris 1× at pH 7.6 or 7.0 buffer using a Zeba spin desalting column according to the manufacturer's protocol (Purification Method 2); or (iii) Monarch® Spin RNA Cleanup kit (50 μg or 500 μg depending on scale) used according to the manufacturers protocol (Purification Method 3). The precursor mRNA and the end product are assayed by RP-HPLC and MS using parameters as shown in Table 4 (eGFP and mCherry) and Table 6 (FLuc).

TABLE 4
RP-HPLC and MS parameters for assaying precursors
and end products of T4 RNA ligation reaction.

| | |
|---|---|
| Column | DNAPac RP column, 2.1 × 100 mm, 4 μm |
| Column Temperature | 85° C. |
| Column flow rate | 0.4 mL/min |
| Solvent A | Water:Triethylamine:HFIP:MeOH v/v/v/v |
| | 477.9:1.05:21.06:0 |
| Solvent B | Water:Triethylamine:HFIP:MeOH v/v/v/v |
| | 227.9:1.05:21.06:250 |
| Gradient | 0.0-1.0 min 0% B, |
| | 1.0-10.0 min 0-36% B, |
| | 10.0-14.0 min 36-52% B, |
| | 14.0-14.5 min 52-0% B, |
| | 14.5-18.0 min 0% B, |
| Mass Spectrometry | Negative electrospray ionization (ESI) |
| Mass Range | 500-2500 m/z |
| Temperature of drying gas | 350° C. |
| Flow rate of drying gas | 13 L/min |
| Pressure of nebulizer gas | 55 psi |
| Sheath gas temperature | 400° C. |
| Sheath gas flow | 12 L/min |
| Capillary positive voltage | 4500 V |
| Capillary negative voltage | 3500 V |
| Nozzle positive voltage | 0 V |
| Nozzle negative voltage | 2000 V |

The following Target mRNA and their starting amount (step (i)) is shown in Table 5.

TABLE 5
Concentrations and volumes of target mRNA to be tested

| | Amount | | | | | Amount | Water |
|---|---|---|---|---|---|---|---|
| Target | added | Concentration | | | Concentration | added | added |
| mRNA | (μL) | (mg/mL) | Buffer | pH | (μM) | (pmol) | (μL) |
| eGFP | 15.3 | 1.00 | 10 mM | 6.5 | 3.13 | 47.8 | 70.4 |
| | | | Citric acid | | | | |
| mCherry | 15.3 | 1.00 | 10 mM | 6.5 | 3.1 | 47.8 | 70.4 |
| | | | Citric acid | | | | |
| FLuc | 29.9 | 0.67 | 10 mM | 6.5 | 1.6 | 47.8 | 55.8 |
| | | | Citric acid | | | | |

The RP-HPLC chromatogram at an absorbance wavelength of 260 nm for eGFP (top) and eGFP-SH (bottom) is shown in FIG. 6B. Over time a new peak (14.84 min) emerges which can be observed by liquid chromatography using the parameters of Table 7. This peak is presumedly a homodimer of eGFP-SH (eGFP-S—S-eGFP) arising from oxidative disulfide formation between the thiols of two mRNA chains. Evidence for this is provided in that exposure of the solutions to DTT reducing conditions (provided in the general procedure) results in the disappearance of this new peak while the original (12.81 min), corresponding to eGFP-SH, remains minimally perturbed (12.82 min). FIG. 6C depicts the RP-HPLC chromatogram at an absorbance wavelength of 260 nm of eGFP-SH after 12 h at 4° C. (top); and eGFP-SH after reduction conditions and isolation using Purification Method 3 (bottom).

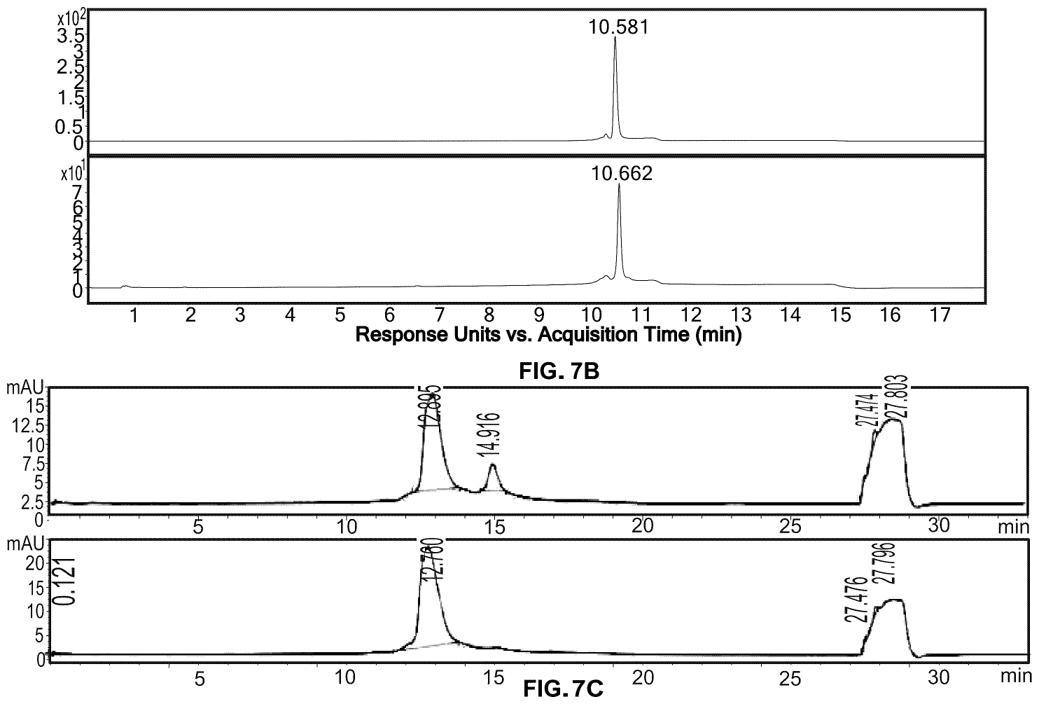

The RP-HPLC chromatogram at an absorbance wavelength of 260 nm for mCherry (top) and mCherry-SH (bottom) is shown in FIG. 7B. Over time a new peak (14.92 min) emerges which can be observed by liquid chromatography using the parameters of Table 7. This peak is presumedly a homodimer of mCherry-SH (mCherry-S—S-mCherry) arising from oxidative disulfide formation between the thiols of two mRNA chains. Evidence for this is provided in that exposure of the solutions to DTT reducing conditions (provided in the general procedure) results in the disappearance of this new peak while the original (12.90 min), corresponding to mCherry-SH, remains minimally perturbed (12.76 min). FIG. 7C depicts the RP-HPLC chromatogram at an absorbance wavelength of 260 nm of mCherry-SH after 12 h at 4° C. (top); and mCherry-SH after reduction conditions and isolation using Purification Method 3 (bottom).

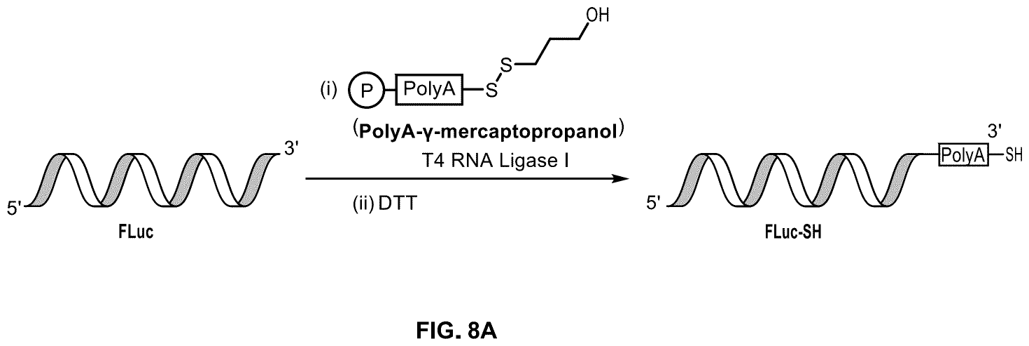

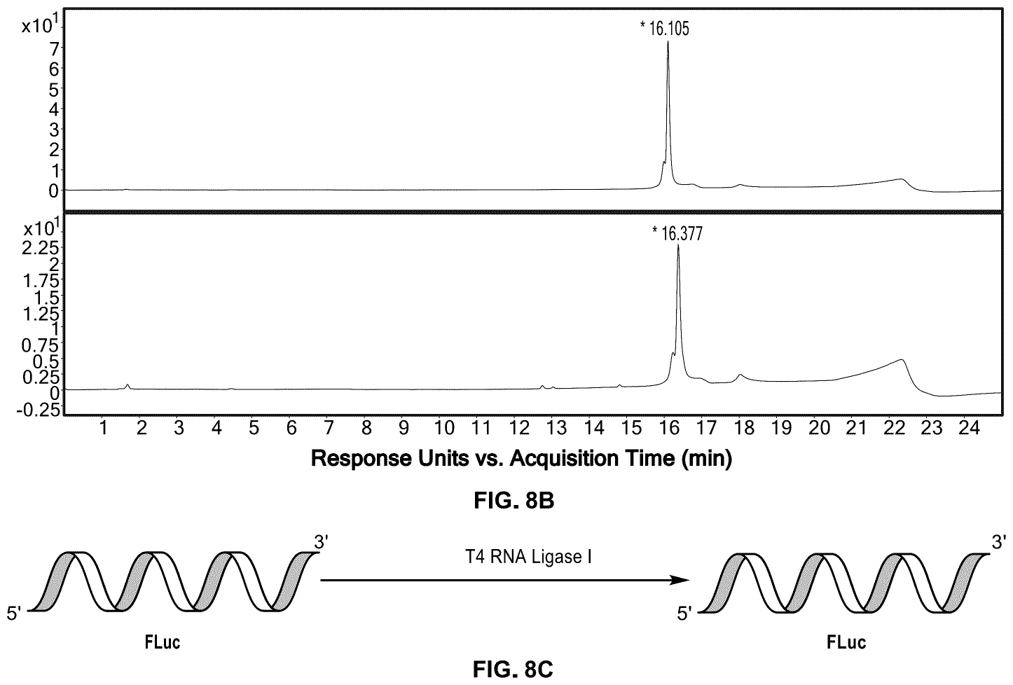

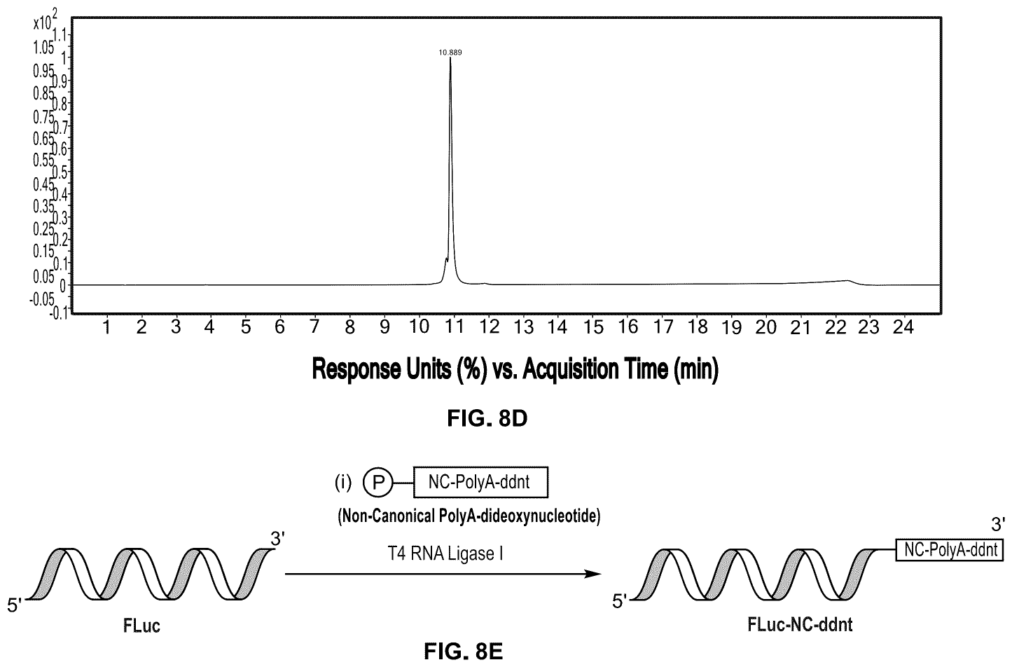

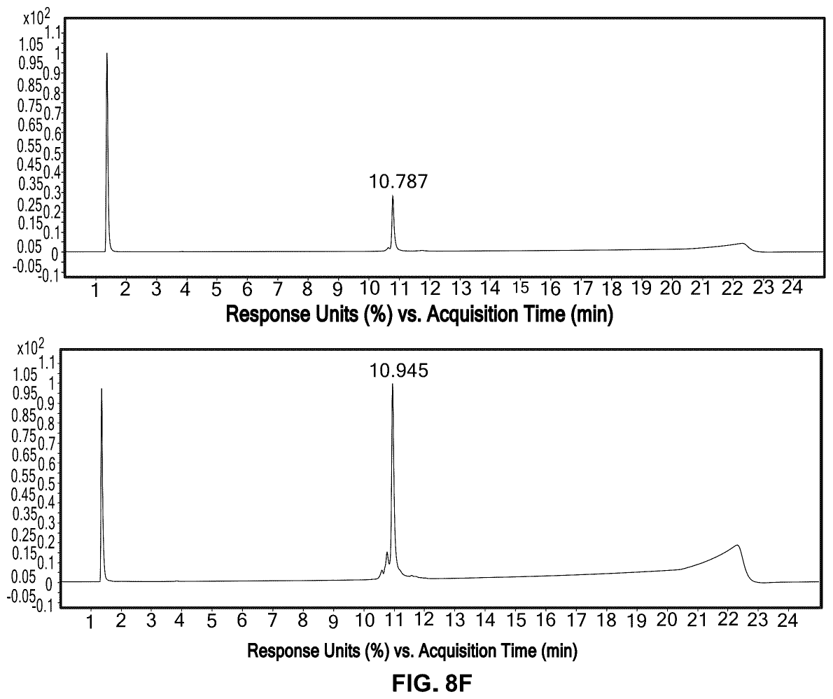

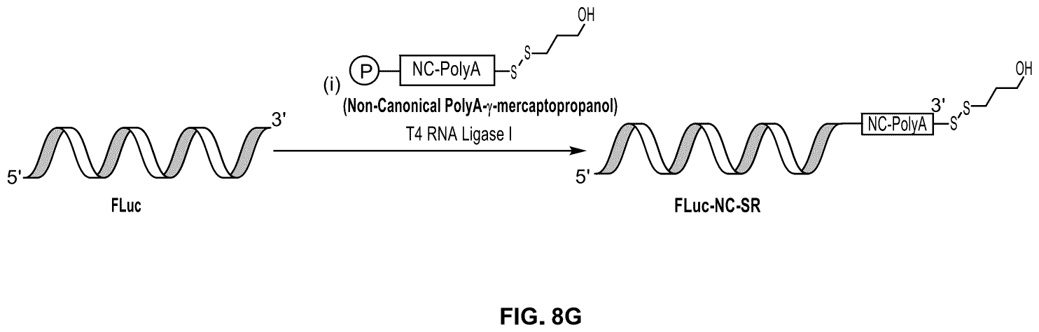

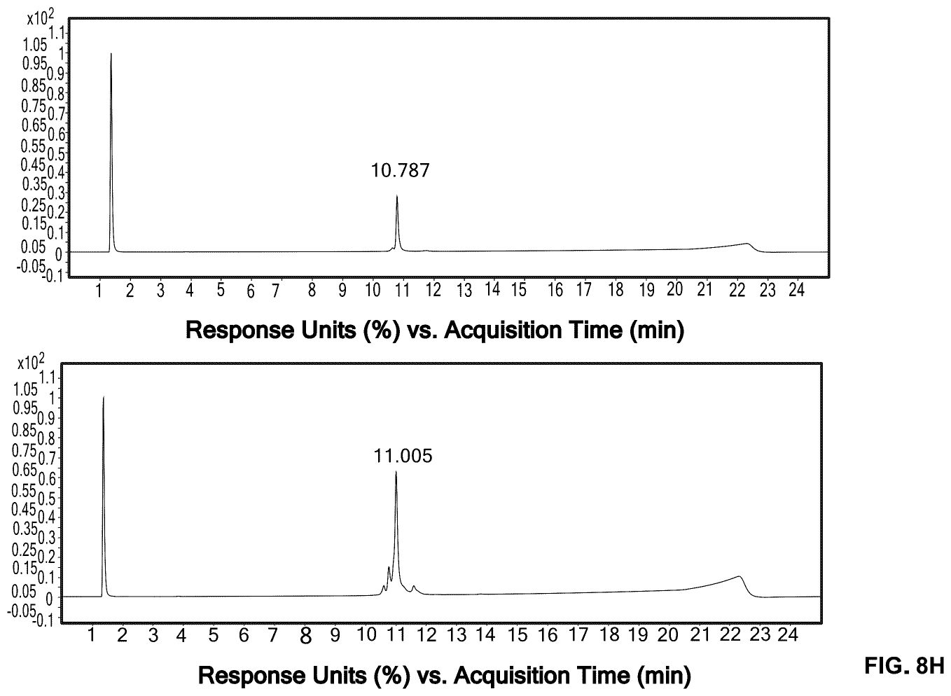

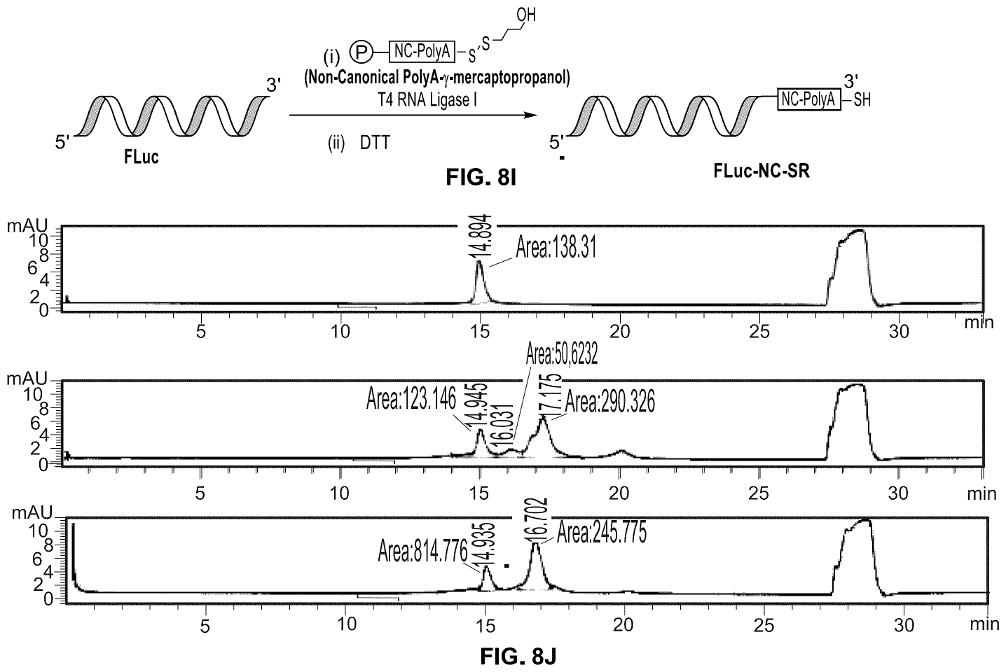

The RP-HPLC chromatogram at an absorbance wavelength of 260 nm for FLuc (top) and FLuc-SH (bottom) is shown in FIG. 8B.

### FLuc (Mock Ligation)

The general procedure for eGFP-SH is followed using FLuc (100 μL, 1.6 μM solution in 10 mM Citric Acid Buffer, pH 6.5) as starting material (FIG. 8C). In this case, PolyA-γ-mercaptopropanol is omitted along with the subsequent reduction step. Purification Method 3 is used to isolate the material (200 μL, 749 nM, 90.2% recovery). FIG. 8D shows the RP-HPLC chromatogram at an absorbance wavelength of 260 nm of FLuc (Mock Ligation), analyzed using the parameters of Table 6.

### FLuc-NC-ddnt

The general procedure for eGFP-SH is followed using FLuc (50 μL, 1.6 μM solution in 10 mM Citric Acid Buffer, pH 6.5) as starting material (FIG. 8E). In this case, non-canonical PolyA-dideoxynucleotide (250 μM, 7.8 μL, 25 equivalents) is used as the oligonucleotide partner for coupling. Purification Method 3 is used to isolate the material (150 μL, 472 nM, 85.6% recovery). FIG. 8F shows the RP-HPLC chromatogram at an absorbance wavelength of 260 nm of FLuc (top) and FLuc-NC-ddnt (bottom), analyzed using the parameters of Table 6.

### FLuc-NC-SR

The general procedure followed for eGFP-SH is followed using FLuc (50 μL, 1.6 μM solution in 10 mM Citric Acid Buffer, pH 6.5) as starting material (FIG. 8G). In this case, non-canonical PolyA-γ-mercaptopropanol (250 μM, 7.8 μL, 25 equivalents) is used as the oligonucleotide partner for coupling. A reduction step with DTT is not performed following the ligation. Purification Method 3 is used to isolate the material (150 μL, 492 nM, 88.7% recovery). FIG. 8H shows the RP-HPLC chromatogram at an absorbance wavelength of 260 nm of FLuc (top) and FLuc-NC-SR (bottom), analyzed using the parameters of Table 6.

### FLuc-NC-SH

The general procedure followed for eGFP-SH is followed using FLuc (100 μL, 1.6 μM solution in 10 mM Citric Acid Buffer, pH 6.5) as starting material (FIG. 8I). In this case, non-canonical PolyA-γ-mercaptopropanol (250 μM, 37.6 μL, 60 equivalents) is used as the oligonucleotide partner for coupling. A reduction step with DTT is not performed following the ligation. Purification Method 3 is used to isolate the material (150 μL, 1.04 μM, 102% recovery). FIG. 8J shows the RP-HPLC chromatogram at an absorbance wavelength of 260 nm of FLuc (top), an intermediate of FLuc-NC-SH after the ligation step but before the DTT reduction step (middle), and FLuc-NC-SH (bottom), analyzed using the parameters of Table 7.

Synthesis of eGFP-S-Maleimide-SulfoCy5

eGFP-S-Maleimide-SulfoCy5 (SEQ ID NO: 98) can be obtained by two different methods.

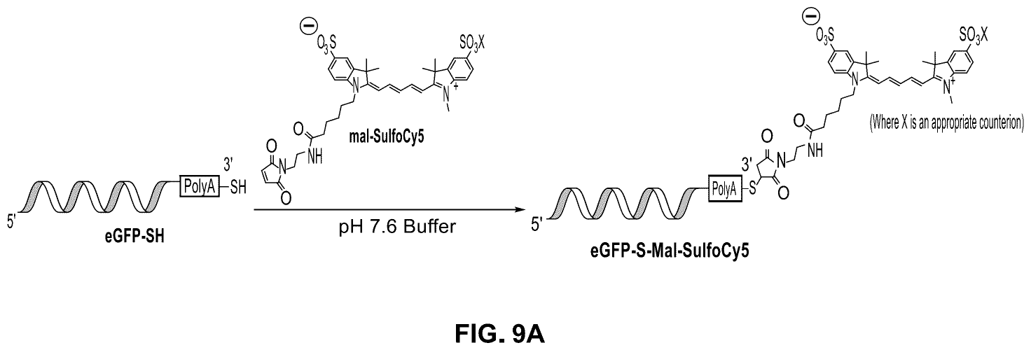

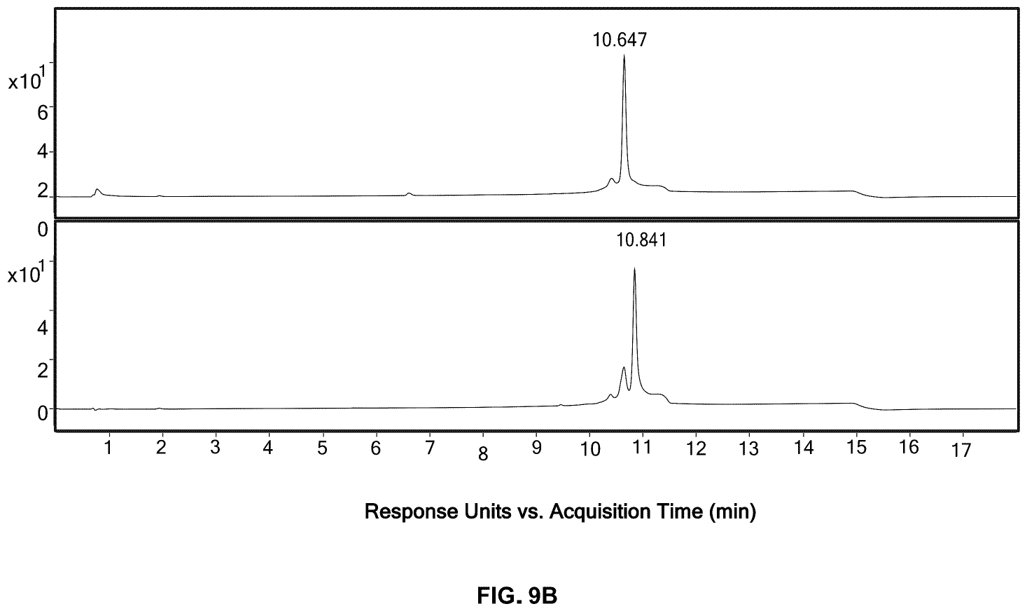

In a first method, eGFP-SH is chemically modified to form eGFP-S-Maleimide-SulfoCy5 (FIG. 9A). Briefly, eGFP-SH (10.0 μL, 100 ng/μL in Tris 1× Buffer, pH 7.6, 1.0 μg) and maleimide-SulfoCy5 (0.6 μL, 250 μM solution in water containing <5% DMSO) are added to a PCR strip tube, and the mixture is mixed by pipetting up and down 20 times. The mixture is incubated at room temperature for 1 hour. The resultant mixture is buffer exchanged/desalted utilizing a Zeba spin micro 40 k MWCO filter according to the manufacturer's protocols. The resulting solution contains eGFP-S-Maleimide-SulfoCy5 (SEQ ID NO: 85; 13 μL, 60.7 ng/μL, 0.79 μg) that is used in subsequent studies without further purification. RP-HPLC is performed using the parameters provided in Table 4. The RP-HPLC chromatogram at an absorbance wavelength of 260 nm for eGFP-SH (top) and eGFP-S-Maleimide-SulfoCy5 (bottom) is shown in FIG. 9B.

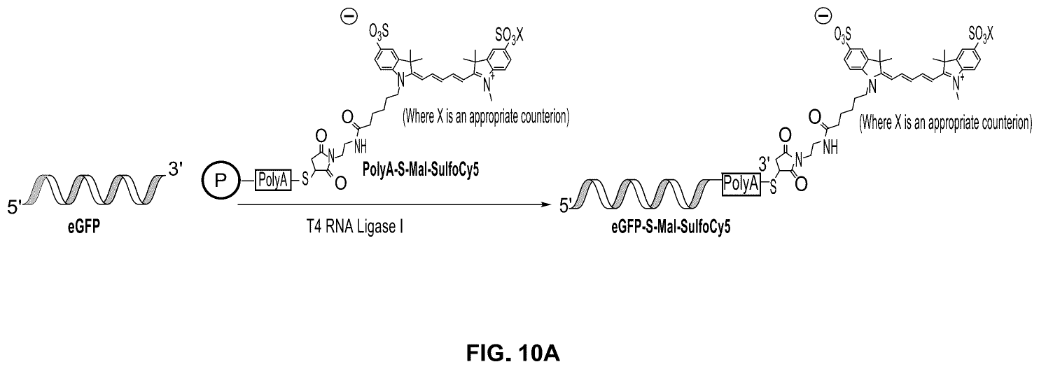

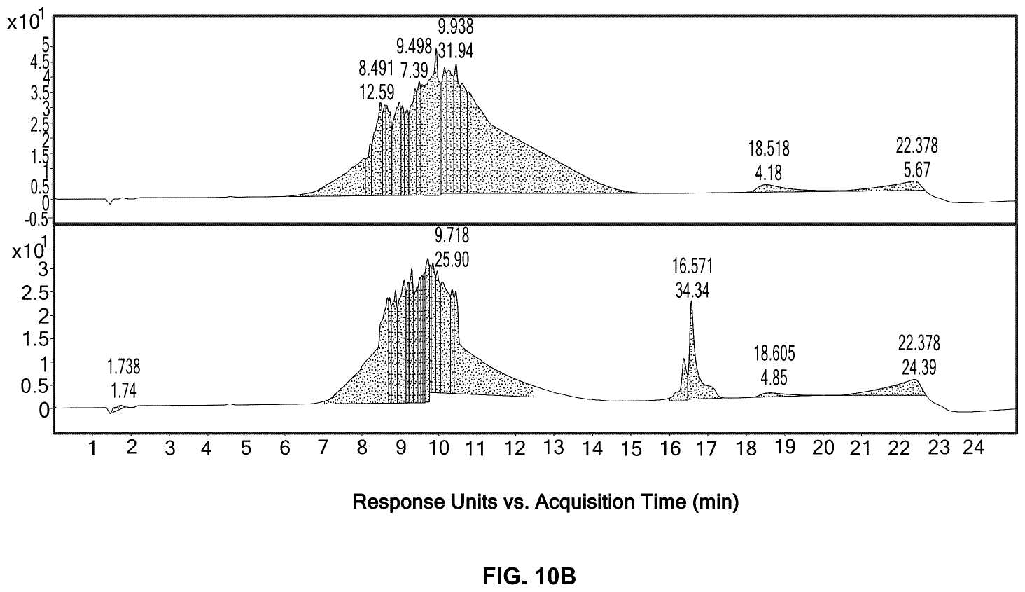

In a second method, eGFP mRNA is enzymatically-ligated to PolyA-S-Maleimide-SulfoCy5 (FIG. 10A), as described above in the section titled “Enzymatic Ligation with T4 RNA Ligase I: General Scheme.” RP-HPLC is performed using the parameters provided in Table 6. The RP-HPLC chromatogram at an absorbance wavelength of 260 nm for eGFP (top) and eGFP-S-Maleimide-SulfoCy5 (bottom) is shown in FIG. 10B.

TABLE 6
RP-HPLC and MS parameters for monitoring
synthesis of eGFP-S-Maleimide-SulfoCy5.

| | |
|---|---|
| Column | DNAPac RP column, 2.1 × 100 mm, 4 μm |
| Column Temperature | 65° C. |
| Column flow rate | 0.2 mL/min |
| Solvent A | Water:Triethylamine:HFIP:MeOH v/v/v/v |
| | 477.9:1.05:21.06:0 |
| Solvent B | Water:Triethylamine:HFIP:MeOH v/v/v/v |
| | 227.9:1.05:21.06:250 |
| Gradient | 0.0-1.0 min 0% B, |
| | 1.0-10.0 min 0-36% B, |
| | 10.0-18.5 min 36-70% B, |
| | 18.5-20.5 min 70-100% B, |
| | 20.5-21.0 min 100-0% B, |
| | 21.0-25.0 min 0% B |
| Mass Spectrometry | Negative electrospray ionization (ESI) |
| Mass Range | 500-2500 m/z |
| Temperature of drying gas | 350° C. |
| Flow rate of drying gas | 13 L/min |
| Pressure of nebulizer gas | 55 psi |
| Sheath gas temperature | 400° C. |
| Sheath gas flow | 12 L/min |
| Capillary positive voltage | 4500 V |
| Capillary negative voltage | 3500 V |
| Nozzle positive voltage | 0 V |
| Nozzle negative voltage | 2000 V |

Synthesis of mCherry-S-Maleimide-SulfoCy5

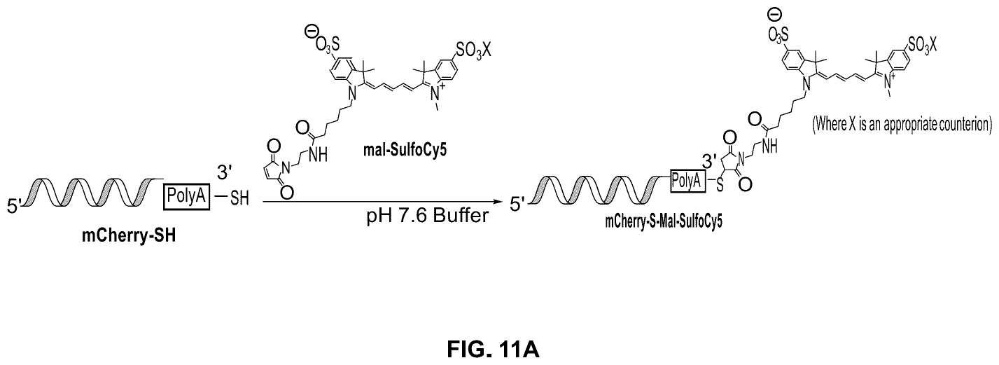

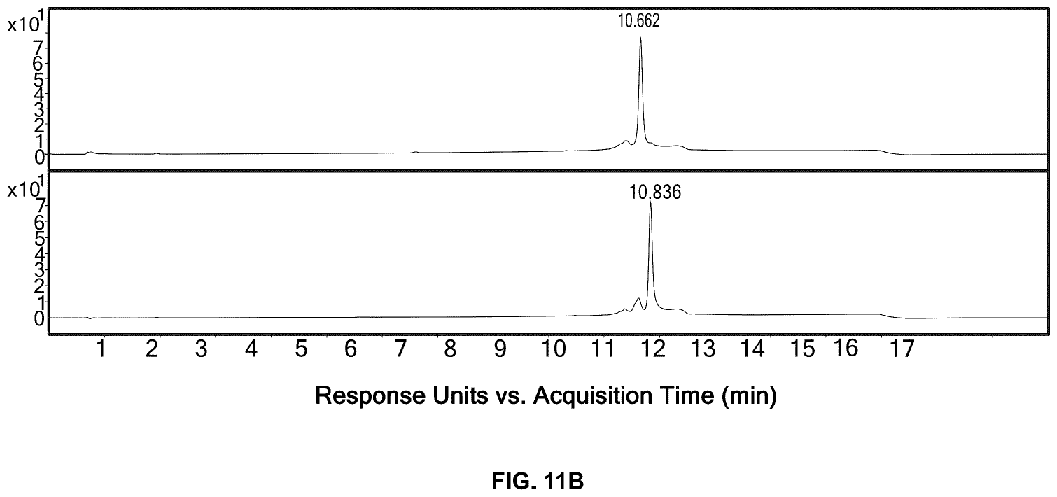

The same method is used to prepare mCherry-S-Maleimide-SulfoCy5 (SEQ ID NO: 86), where instead of starting with eGFP-SH, mCherry-SH (SEQ ID NO: 97) is used instead (FIG. 11A). RP-HPLC is performed using the parameters provided in Table 4. The RP-HPLC chromatogram at an absorbance wavelength of 260 nm for mCherry-SH (top) and mCherry-S-Maleimide-SulfoCy5 (bottom) is shown in FIG. 11B.

### Synthesis of FLuc-S-Maleimide-SulfoCy5

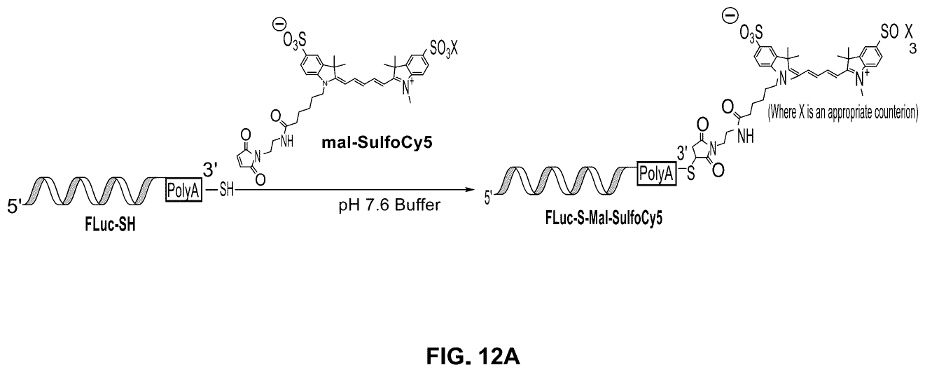

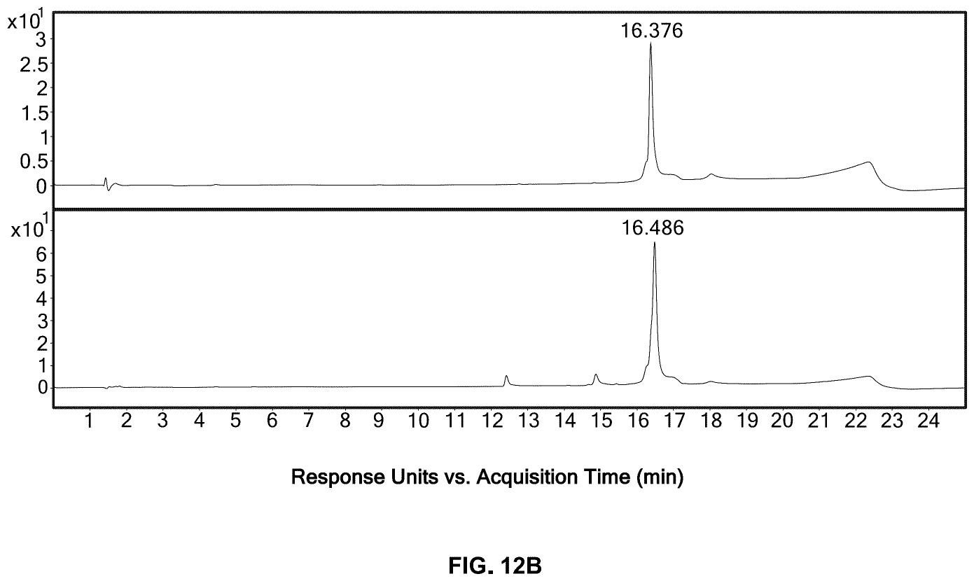

The same chemical modification method is also used to prepare FLuc-S-Maleimide-SulfoCy5 (FIG. 12A), where instead of starting with eGFP-SH, FLuc-SH (40 μL, 99.8 ng/μL, 4.0 μg) is used instead. RP-HPLC is performed using the parameters provided in Table 6. After buffer exchange, the resulting solution of FLuc-S-Maleimide-SulfoCy5 (SEQ ID NO: 87; 70 μL, 34.1 ng/μL, 2.4 μg). The RP-HPLC chromatogram at an absorbance wavelength of 260 nm for FLuc-SH (top) and FLuc-S-Maleimide-SulfoCy5 (bottom) is shown in FIG. 12B.

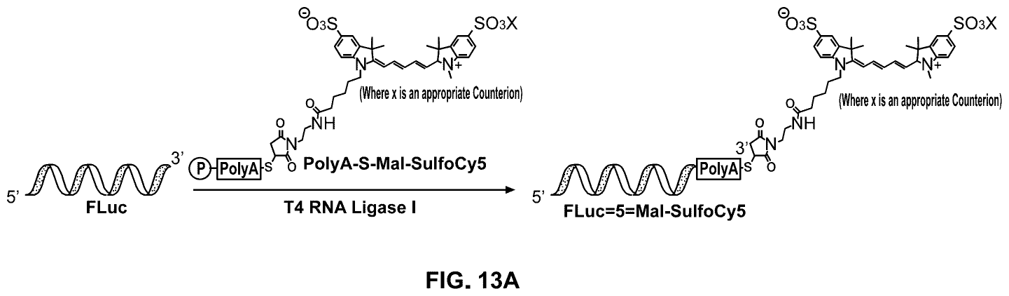

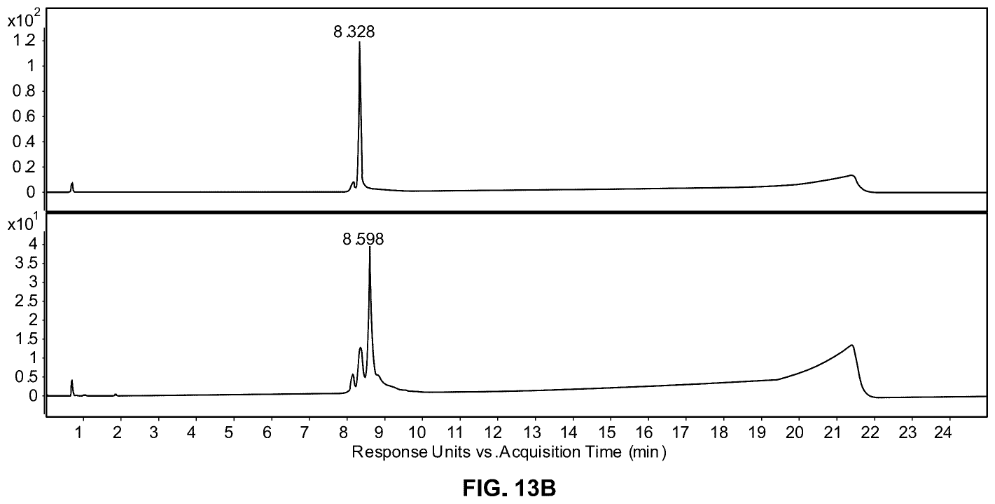

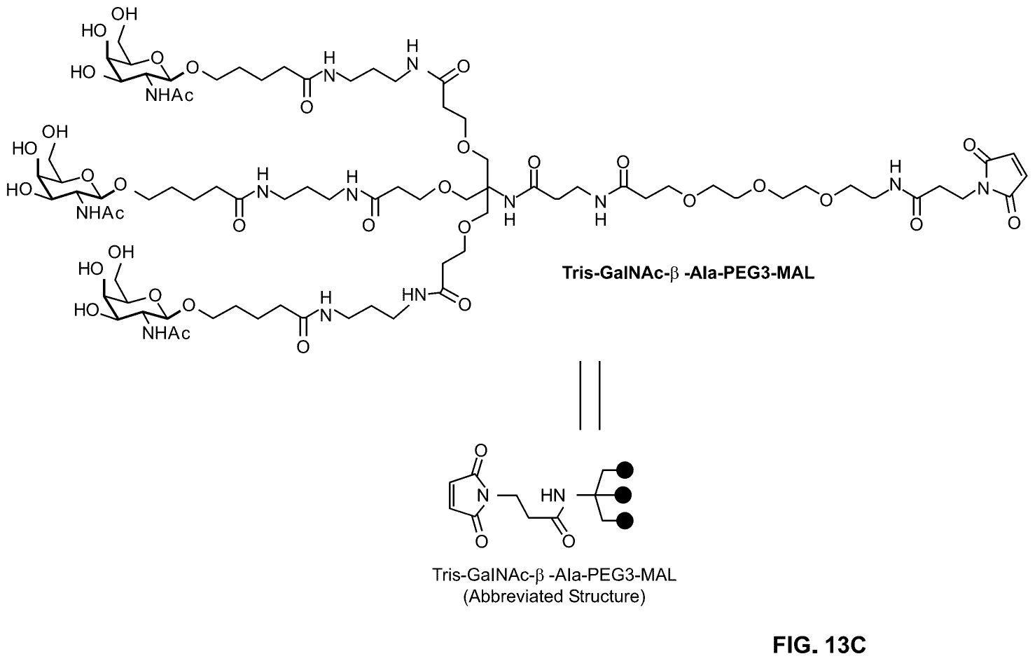

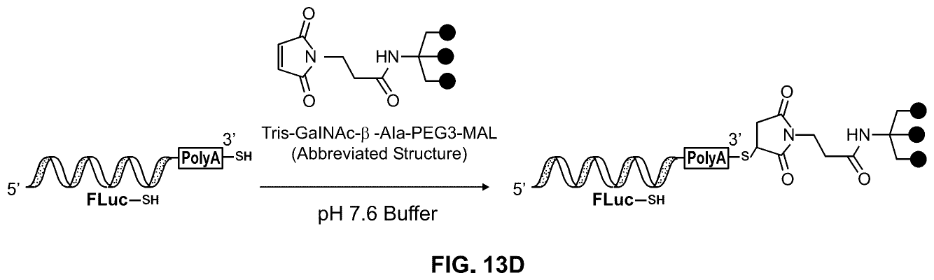

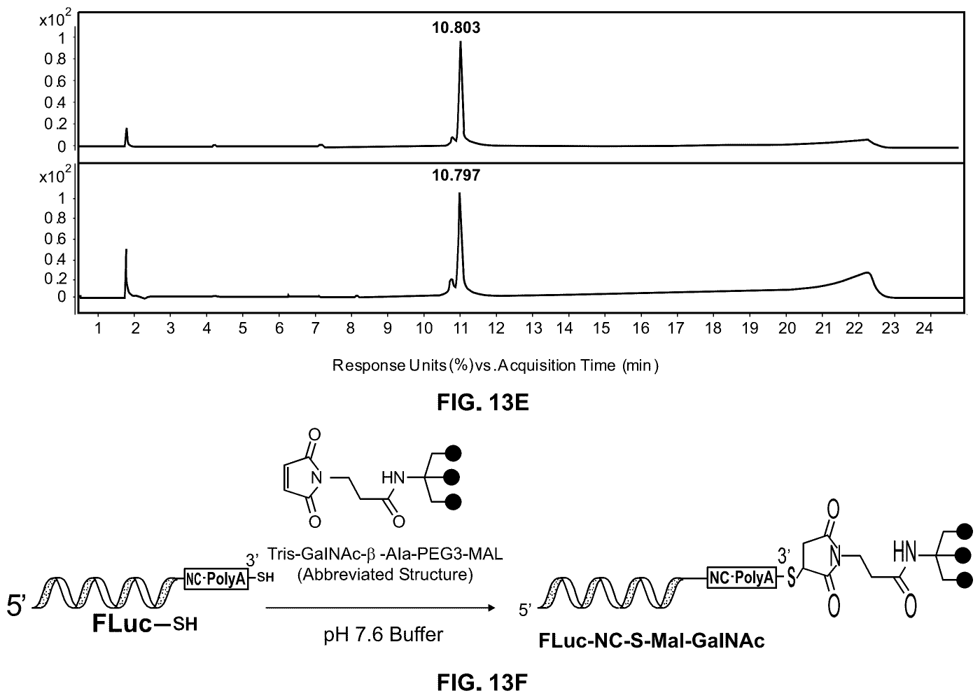

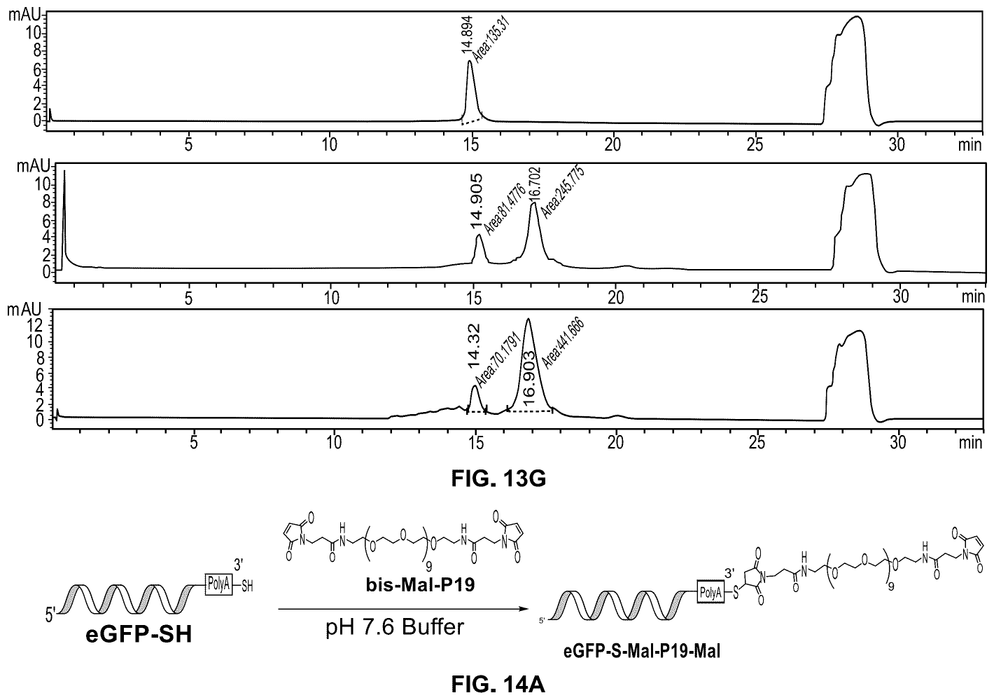

FLuc-S-Maleimide-SulfoCy5 can also be prepared using enzymatic ligation as described above (FIG. 13A), and instead using PolyA-S-Maleimide-SulfoCy5 (25.5 μL, 195 μM stock in Tris 1× buffer, pH 7) and FLuc (72.5 μL, 48.5 μg, 0.667 mg/mL in water, 78 μmol). From this, FLuc-S-Maleimide-SulfoCy5 (200 μL, 201.6 ng/μL, 40.3 μg) is obtained. RP-HPLC is performed using the parameters provided in Table 3. The RP-HPLC chromatogram at an absorbance wavelength of 260 nm for FLuc (top) and FLuc-S-Maleimide-SulfoCy5 (bottom) is shown in FIG. 13B.

### Synthesis of FLuc-S-Maleimide-GalNAc

Tris-GalNAc-β-Ala-PEG3-Maleimide is purchased from Sussex Research Laboratories and used directly. The structure and abbreviated structure are depicted in FIG. 13C and FIG. 13D respectively. The preparation of FLuc-S-Maleimide-GalNAc is carried out in an identical manner to the preparation of eGFP-S-Maleimide-SulfoCy5 above, except using FLuc-SH (73.3 μL, 50 μg, 78 μmol) as starting material and Tris-GalNAc-β-Ala-PEG3-Maleimide (8.00 μL, 1000 μM in DMSO). The reaction is allowed to incubate at room temperature for 15 minutes before the entire reaction mixture is purified using a Monarch® Spin RNA Cleanup kit (50 μg) according to the manufacturer's protocol. The isolated material is eluted into 100 μL of nuclease free water, providing a solution of FLuc-S-Maleimide-GalNAc (100 μL, 337 ng/μL, 67.4% recovery). The RP-HPLC chromatogram performed using the parameters of Table 6 at an absorbance wavelength of 260 nm for FLuc-SH (top) and FLuc-S-Maleimide-GalNAc (bottom) is shown in FIG. 13E.

### Synthesis of FLuc-NC-S-Maleimide-GalNAc

The preparation of FLuc-NC-S-Maleimide-GalNAc (FIG. 13F) is carried out in an identical manner to the preparation of eGFP-S-Maleimide-SulfoCy5 above, except using FLuc-NC-SH (SEQ ID NO: 84; 73.3 μL, 50 μg, 78 μmol) as starting material and Tris-GalNAc-3-Ala-PEG3-Maleimide (8.00 μL, 1000 μM in DMSO). The reaction is allowed to incubate at room temperature for 15 minutes before the entire reaction mixture is purified using a Monarch® Spin RNA Cleanup kit (50 μg) according to the manufacturer's protocol. The isolated material is eluted into 100 μL of nuclease free water, providing a solution of FLuc-NC-S-Maleimide-GalNAc (SEQ ID NO: 89; 100 μL, 388 ng/μL, 77.6% recovery). The RP-HPLC chromatogram performed using the parameters of Table 7 at an absorbance wavelength of 260 nm for a persistent impurity FLuc (top), FLuc-NC-SH (middle), and FLuc-S-Maleimide-GalNAc (bottom) is shown in FIG. 13G.

Synthesis of eGFP-S-Maleimide-PEG19-Maleimide

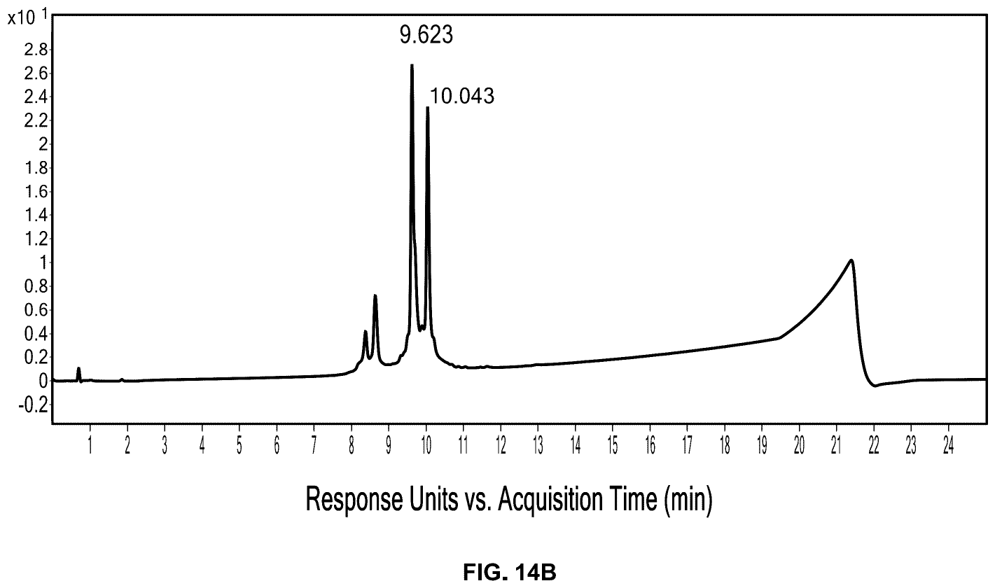

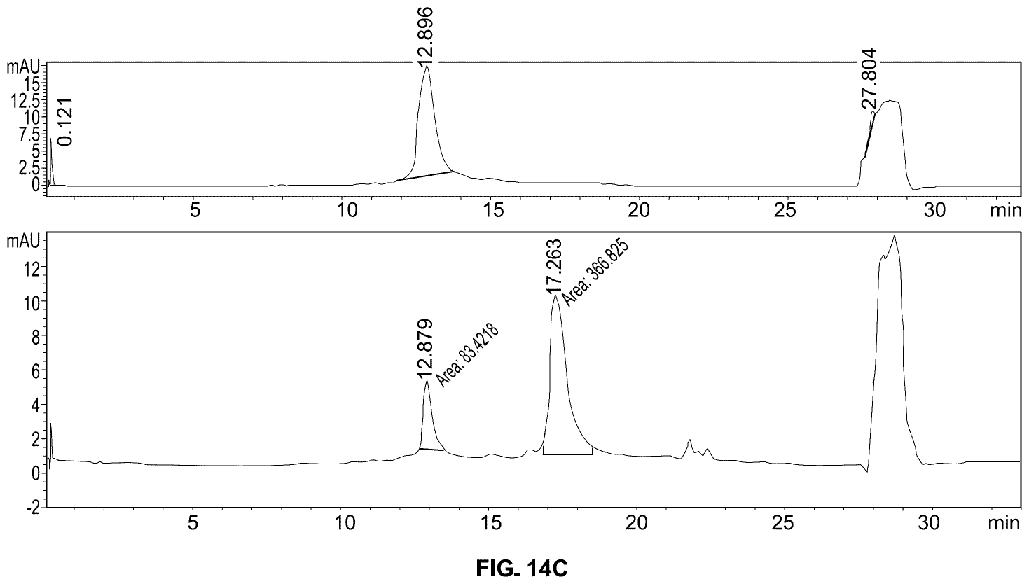

eGFP-S-Maleimide-PEG19-Maleimide is synthesized from purified eGFP-SH derived as described herein (FIG. 14A). Briefly, eGFP-SH (75 μL, 177 ng/μL in Tris 1× pH 7.6 buffer, 13.3 μg, 41.5 μmol) and bis-Maleimide-PEG19 (2.1 μL, 1.0 mM in 9:1 water:DMSO) are added to a 0.5 mL Eppendorf tube. The mixture is pipetted up and down 20 times so as to mix the reaction, and incubated at room temperature for 1.5 hours after which it is purified by a Zeba spin 0.5 mL, 40 k MWCO desalting column according to the manufacturers protocols. The resulting solution of eGFP-S-Maleimide-PEG19-Maleimide (SEQ ID NO: 90; 90 μL, 138.3 ng/μL in Tris 1× buffer, pH 7.6) is used in subsequent studies/experiments without further purification or could be further purified using a centrifugal spin concentrator with a MWCO of ≤100 kDa. RP-HPLC is performed using the parameters provided in Table 3.

As shown in FIG. 14B, the RP-HPLC UV absorption chromatogram appears to have two predominant peaks (9.623 min and 10.043 min) when analyzed with the HPLC parameters of Table 3. Peaks to the left of the main peak correspond to eGFP-SH. The two peaks are the result of the specific liquid chromatography method used, as evidenced by the different results when the analysis is performed under different temperatures.

FIG. 14C provides an alternative chromatogram, analyzing the same eGFP-S-Maleimide-PEG19-Maleimide using parameters specified in Table 7. Here, There is likely less hydrolysis as the product appears as a single peak at 17.263 min. The top panel of FIG. 14C shows the RP-HPLC chromatogram of eGFP-SH while the bottom panel of FIG. 14C shows the RP-HPLC chromatogram of the isolated eGFP-S-Maleimide-PEG19-Mal.

Synthesis of mCherry-S-Maleimide-PEG19-Maleimide

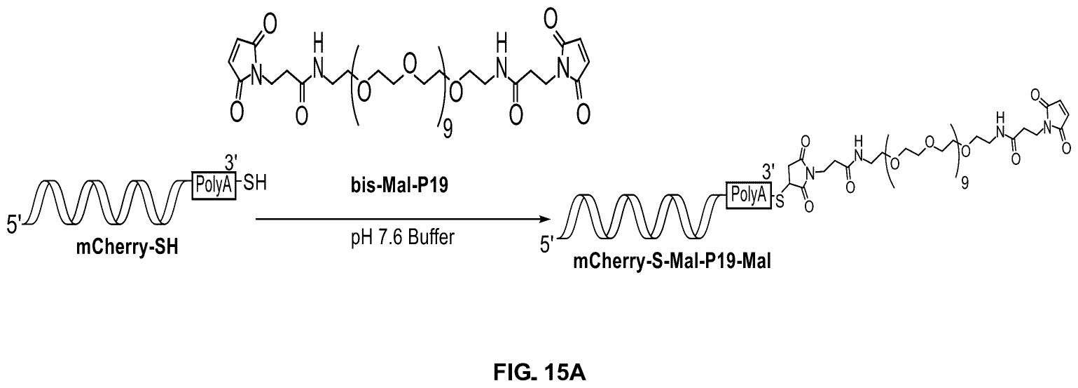

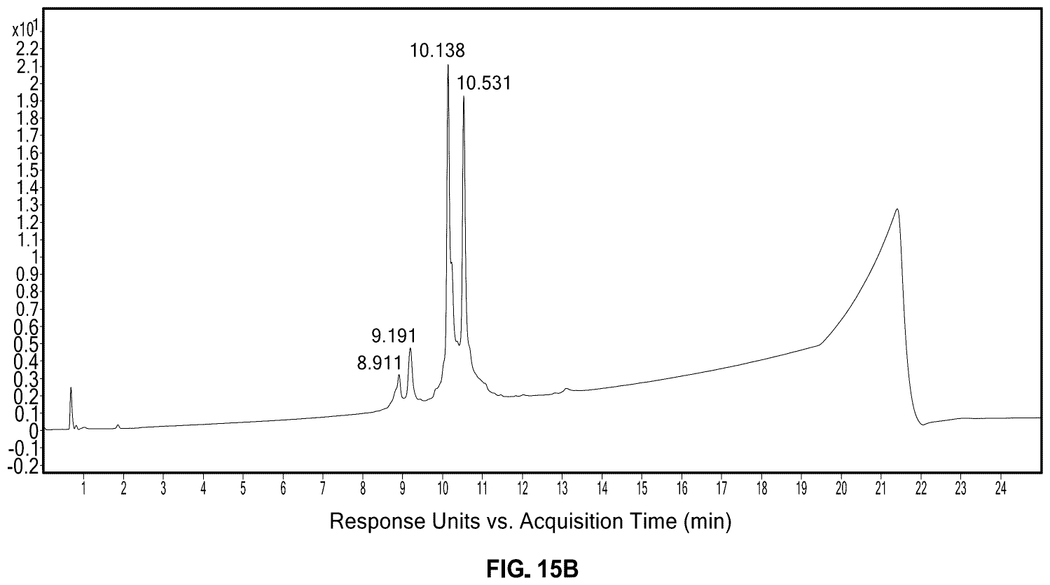

mCherry-S-Maleimide-PEG19-Maleimide is synthesized from purified mCherry-SH (SEQ ID NO: 97) derived as described herein (FIG. 15A). Briefly, mCherry-SH (110 μL, 125.0 ng/μL, in Tris 1× pH 7.0 buffer, 13.8 μg, 43.0 μmol) and bis-Maleimide-PEG19 (2.5 μL, 1.0 mM solution in 9:1 water:DMSO) are added to a 0.5 mL Eppendorf tube. The mixture is pipetted up and down 10 times so as to mix the reaction. The reaction is monitored by RP-HPLC using the parameters of Table 3. After 2 h 55 min, bis-Maleimide-PEG19 (10.0 μL, 1.0 solution in 9:1 water:DMSO) is added followed by Tris 10× buffer (5.0 μL, pH 7.0) and the solution mixed by pipetting up and down 10 times. The mixture is incubated for another 22 min after which additional bis-Maleimide-PEG19 (5.0 μL, 1.0 mM solution in 9:1 water:DMSO) is added and the solution mixed by pipetting up and down 10 times. The mixture is incubated for an additional 8 min, after which it is purified by a Zeba spin 0.5 mL 40 k MWCO desalting column according to the manufacturers protocols. The recovered mCherry-S-Maleimide-PEG19-Maleimide (135 μL, 95.9 ng/μL) is used without further purification or can be further purified using a centrifugal spin concentrator with a MWCO of ≤100 kDa.

As shown in FIG. 15B, the RP-HPLC UV absorption chromatogram appears to have two predominant peaks at 10.138 min and 10.531 min when analyzed with the HPLC parameters of Table 3. The main peaks correspond to mCherry-S-Maleimide-PEG19-Maleimide in the hydrolyzed form (see experiment on varying temperature of column). Peaks to the left of the main peak at 8.911 min and 9.191 min correspond to residual mCherry-SH. The right shoulder of the peak at 10.138 min is tentatively assigned as the homodimer of mCherry-SH (e.g., mCherry-S-Maleimide-PEG19-Maleimide-S-mCherry) or an adduct formed from reaction with maleimide and hexafluoroisopropalnol (HFIP).

Reaction of mCherry-S-Maleimide-PEG19-Maleimide with Small Molecule Thiols or a Oligonucleotide Containing a Thiol

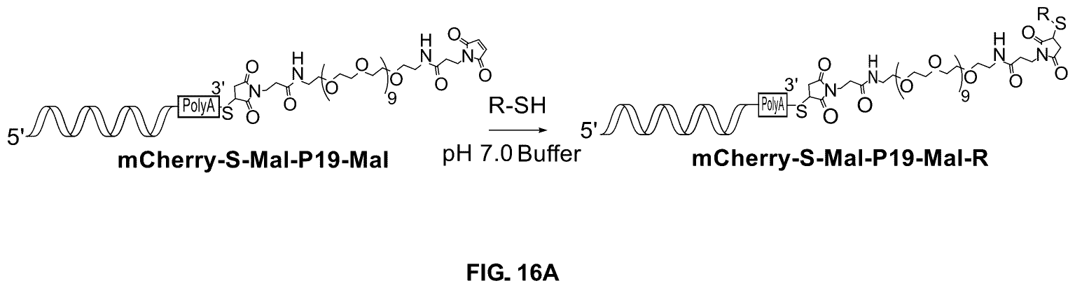

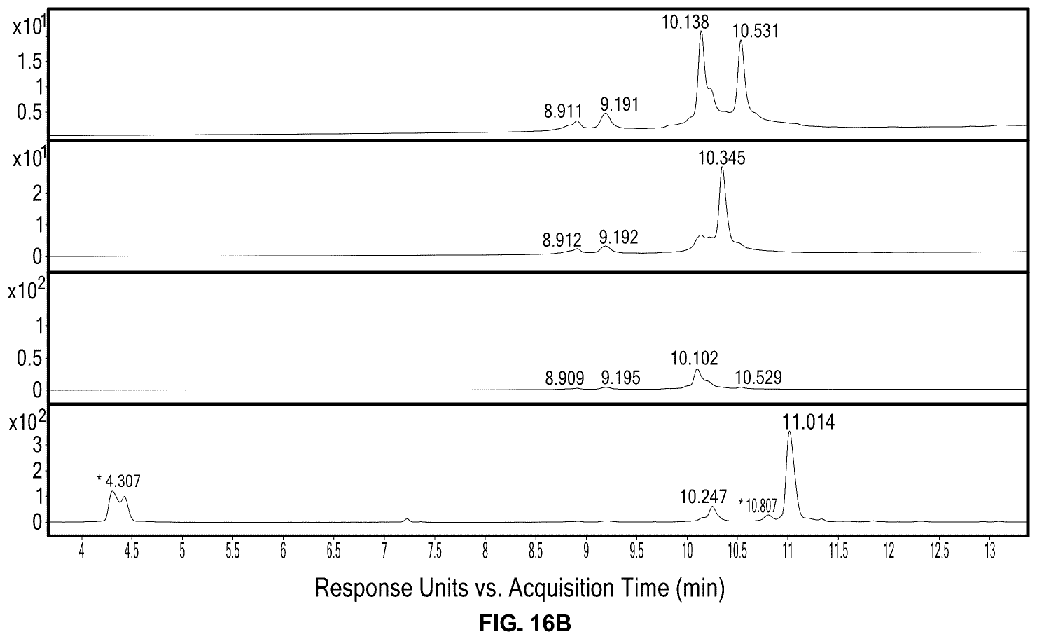

mCherry-S-Maleimide-PEG19-Maleimide can be further conjugated with thiol-containing oligonucleotides or small molecules (FIG. 16A). Briefly, mCherry-S-Maleimide-PEG19-Maleimide (SEQ ID NO: 91; 2.0 μL, 95.9 ng/μL) is added to a vial, followed by the addition of either: (i) beta-mercaptoethanol (10 μL, 285 μM in Tris 1×, pH 7.0; (ii) Cysteine (10 μL, 285 μM in Tris 1×, pH 7.0) or (iii) Tris 1× buffer (8.5 μL, pH 7.0) followed by PolyA-SH (1.5 μL, 1396.2 ng/μL). The solutions are mixed by pipetting up and down. The solutions are incubated for 45 min after which conversion is assessed by RP-HPLC monitoring at a wavelength of 260 nm using the HPLC parameters of Table 3. FIG. 16B shows the chromatograms for mCherry-S-Maleimide-PEG19-Maleimide (topmost), reaction with beta-mercaptoethanol (second from top), reaction with Cysteine (second from bottom) and PolyA-SH (bottommost). Peaks at 4.307 min and 11.014 min correspond to residual PolyA-SH and PolyA-dimer, respectively, which can be removed via MWCO filters.

### Synthesis of FLuc-S-Maleimide-PEG19-Maleimide

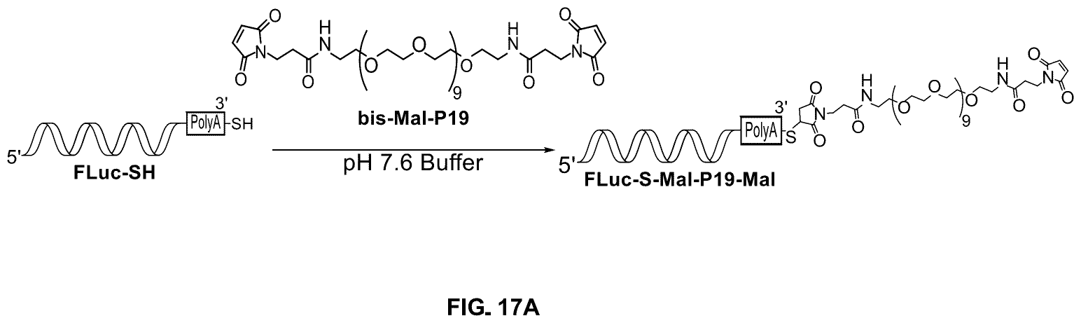

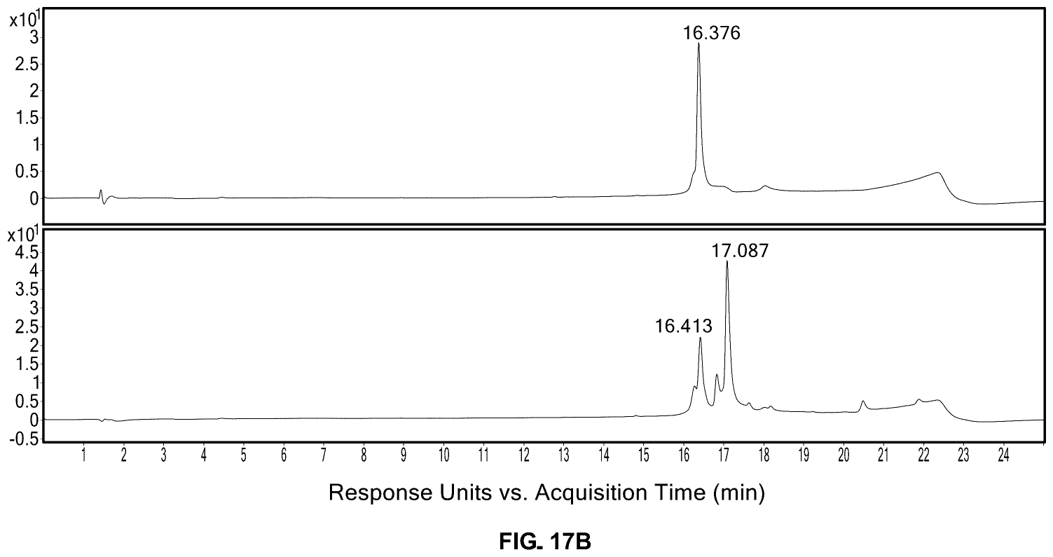

The same method is used to prepare FLuc-S-Maleimide-PEG19-Maleimide (SEQ ID NO: 92) (FIG. 17A) as eGFP-S-Maleimide-PEG19-Maleimide above, where instead of starting with eGFP-SH, FLuc-SH is used instead. The reaction is monitored by HPLC with the parameters listed in Table 6. As shown in FIG. 17B, the mixture appears as two predominant peaks at 16.413 min and 17.087 min. The two peaks are a result of the liquid chromotography method, as evidenced by carrying out liquid chromatography analysis of analogous compounds under varying temperature analysis.

Synthesis of eGFP-S-Maleimide-PEG19-Maleimide-A-PolyA

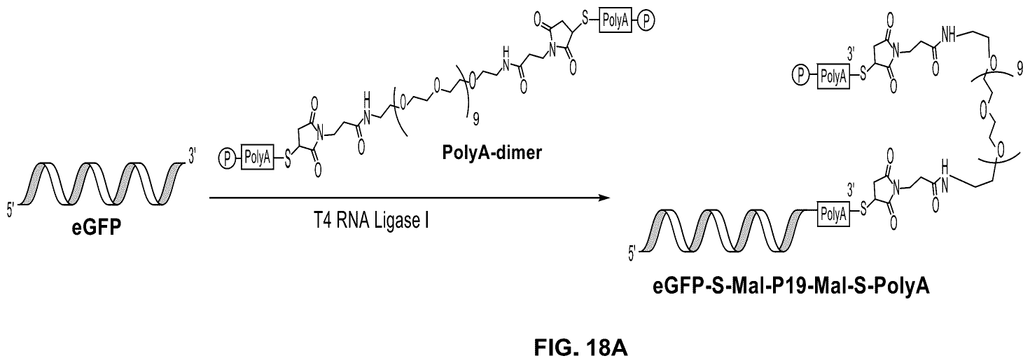

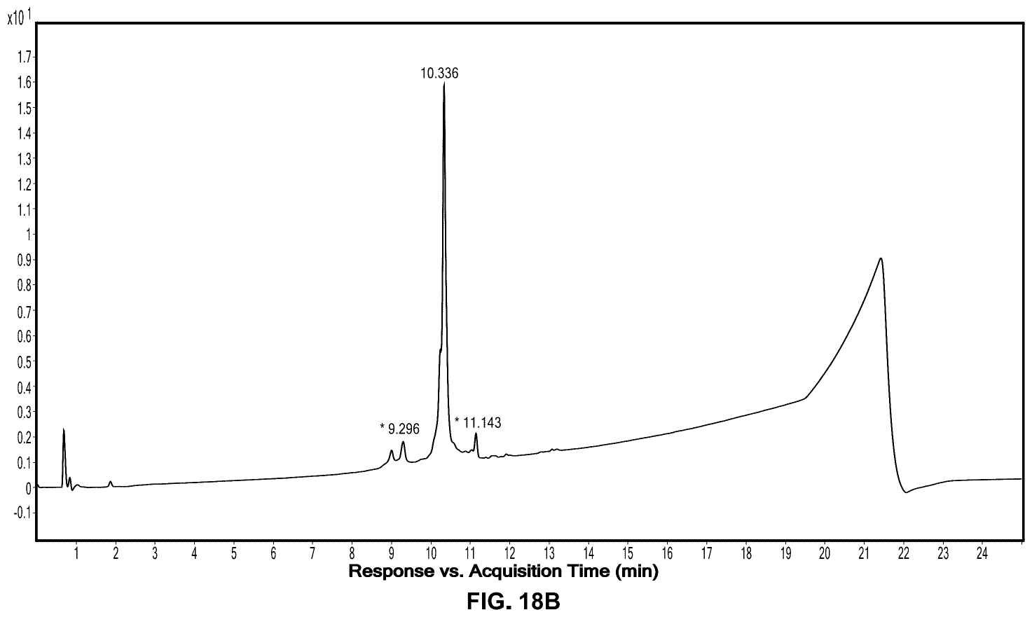

The representative T4 RNA Ligase I reaction protocol described herein is carried out using eGFP (5.0 μL, 1 mg/mL in 10 mM citrate, pH 6.5) and PolyA-dimer (11.2 μL, 88 μM in Tris 1×, pH 7.0) (FIG. 18A). After purification by AMPure XP magnetic bead, eGFP-S-Maleimide-PEG19-S-PolyA (50 μL, 61.3 ng/μL) is recovered. The material is buffer exchanged 4 times using a centrifugal spin concentrator with a MWCO of ≤100 kDa by diluting in 200 μL of Tris 1×, pH 7.0 buffer and spin concentrating for ˜2 min at 10 k rcf to give a supernatant volume of ˜50 μL. A total of 25 μL of the mixture containing eGFP-S-Maleimide-PEG19-S-PolyA is recovered at a concentration of 29.8 ng/μL. FIG. 18B shows the UV absorption chromatogram of eGFP-S-Maleimide-PEG19-S-PolyA when assessed with the HPLC parameters of Table 3. The left shoulder of the peak at 10.336 min corresponds well with eGFP-S-Maleimide-PEG19-S-eGFP.

Synthesis of eGFP-S-Maleimide-PEG19-Maleimide-S-eGFP

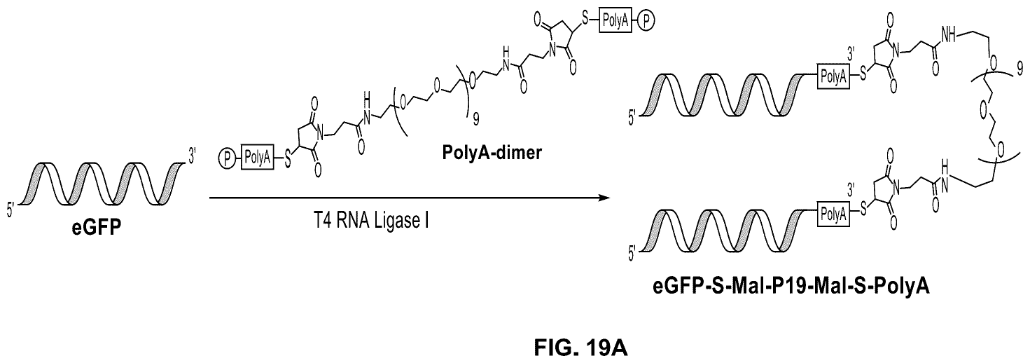

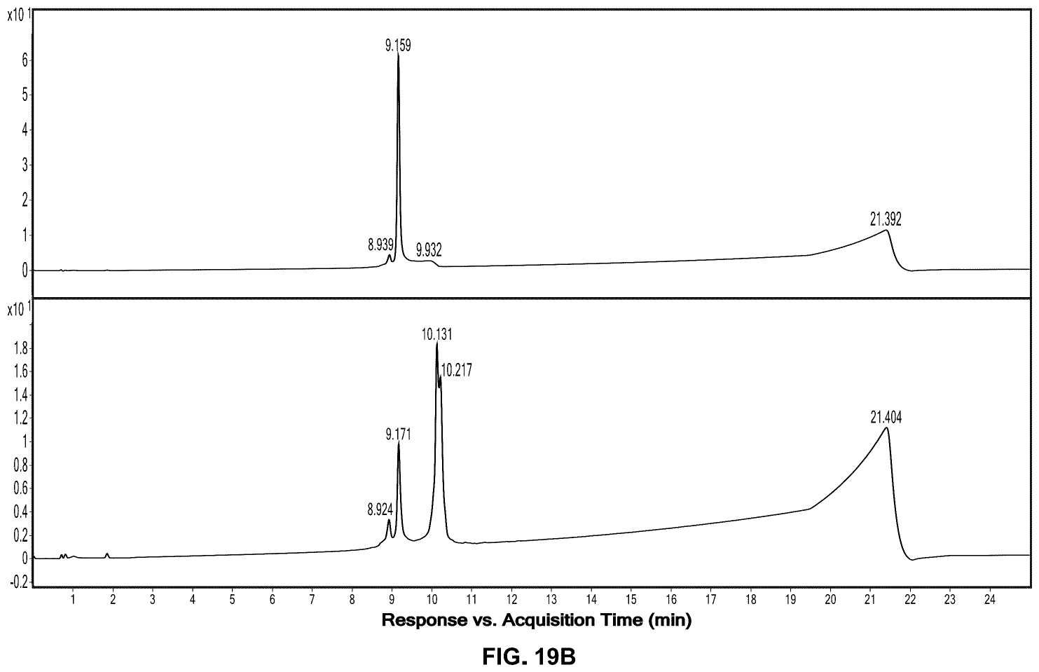

The representative T4 RNA Ligase I reaction protocol described herein is carried out using eGFP (5.1 μL, 1 mg/mL in 10 mM citrate, pH 6.5) and PolyA-dimer (1.77 μL, 8.8 μM in Tris 1×, pH 7.0) (FIG. 19A). After purification by AMPure XP magnetic bead, eGFP-S-Maleimide-PEG19-S-eGFP (50 μL, 41.5 ng/μL) is recovered. FIG. 19B (top) shows the UV absorption chromatogram of eGFP. FIG. 19B (bottom) shows the reaction of eGFP with PolyAdimer to give eGFP-S-Maleimide-PEG19-Maleimide-S-eGFP (SEQ ID NO: 94) when assessed with the HPLC parameters of Table 3. The obtained material is consistent with a mixture of eGFP (peaks at 8.924 min and 9.171 min), eGFP-S-Maleimide-PEG19-S-eGFP (peak at 10.131 min) and eGFP-S-Maleimide-PEG19-S-Maleimide-PolyA (peak at 10.217 min).

Synthesis of eGFP-S-Maleimide-PEG19-Maleimide-S-mCherry

eGFP-S-Maleimide-PEG19-Maleimide-S-mCherry (SEQ ID NO: 96) can be obtained by four different methods.

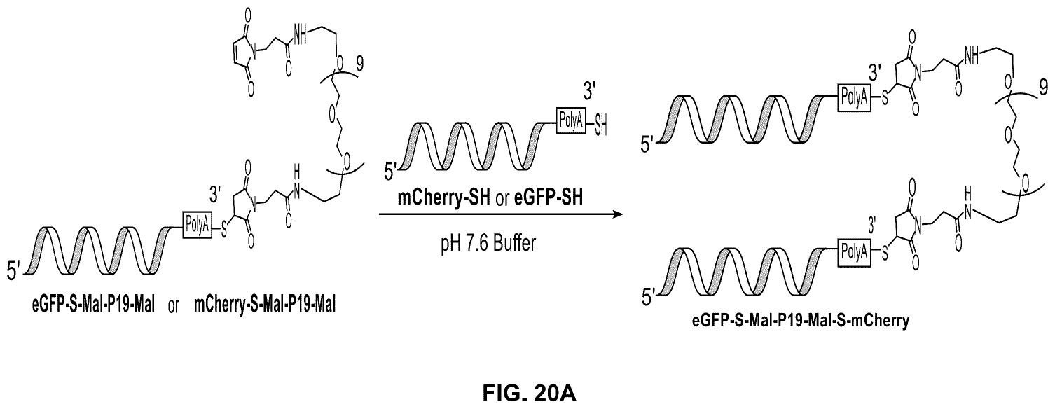

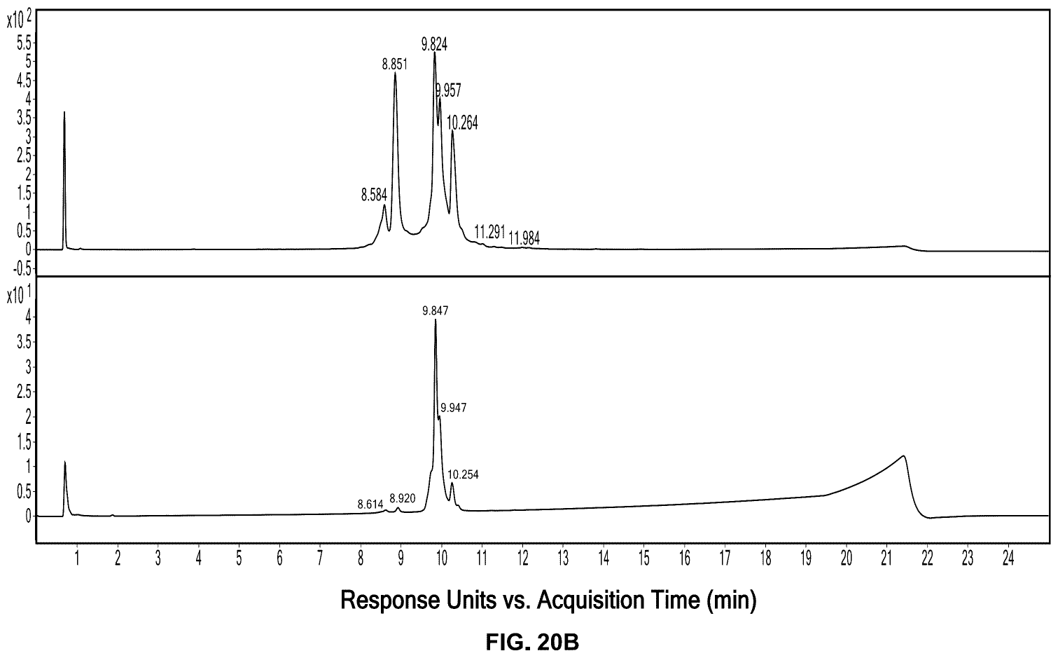

In a first method, eGFP-S-Maleimide-PEG19-Maleimide is treated with mCherry-SH (FIG. 20A). Briefly, eGFP-S-Maleimide-PEG19-Maleimide (15 μL, 138.3 ng/μL) is added to a 0.5 mL Eppendorf tube, followed by mCherry-SH (22 μL, 188 ng/μL). The solution mixed by pipetting up and down 10 times. The mixture is transferred to a centrifugal spin concentrator with a MWCO of 50 kDa and concentrated to a total volume of ˜22 μL. The mixture is incubated at room temperature for 2.5 hours and further incubated at 4° C. for ˜14 hours. Finally, the material is purified by RP-HPLC and fractions containing the latter three peaks are collected and buffer exchanged into citric acid buffer (50 mM citric acid, pH 5.5). A total of 31 μL of the mixture containing eGFP-S-Maleimide-PEG19-S-mCherry at a concentration of 42.9 ng/μL is recovered. This reaction is monitored by HPLC with the parameters listed in Table 3. FIG. 20B shows the UV absorption chromatogram of the crude reaction mixture (top) and the purified mixture used in subsequent assays (bottom). The crude mixture shows peaks at 8.584 min and 8.851 min that correspond to mCherry-SH and peaks at 9.824 min and 10.264 min that correspond to eGFP-S-Maleimide-PEG19-Maleimide. The purified mixture shows less than 5% residual mCherry-SH.

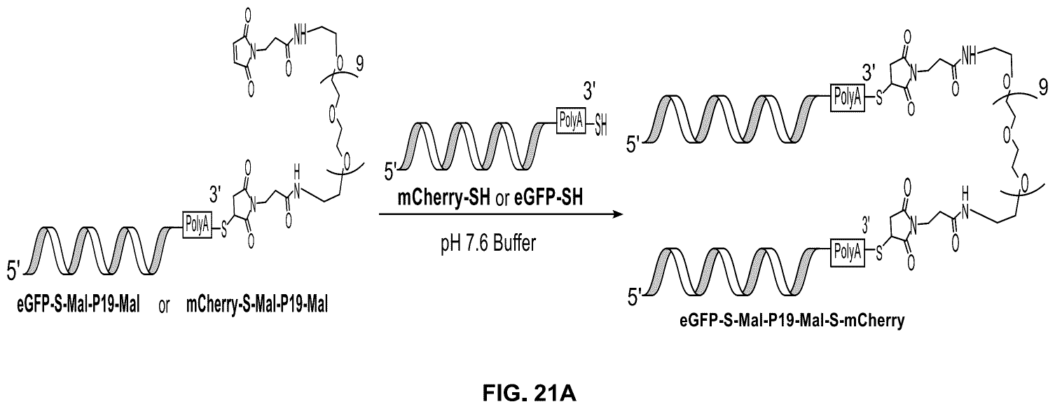

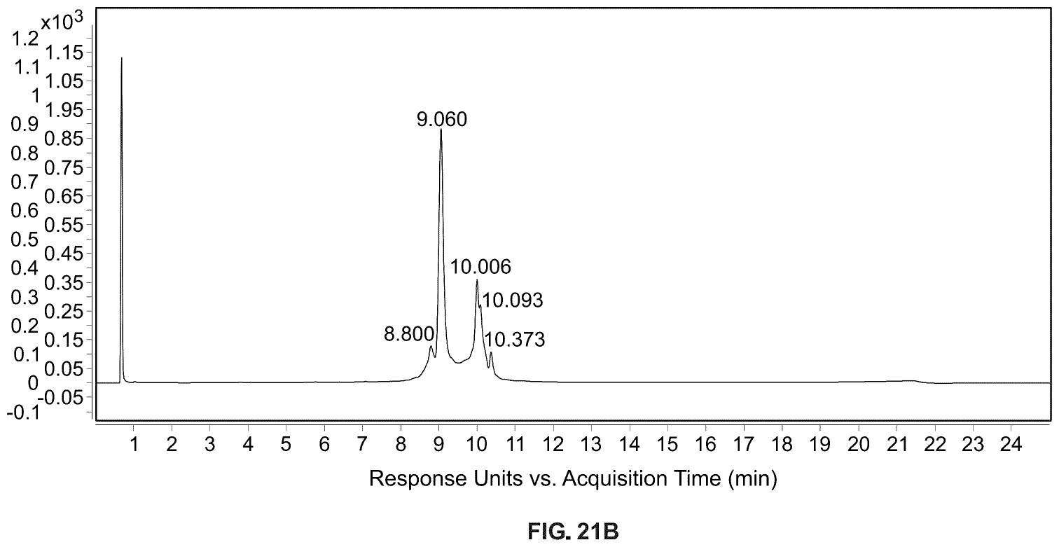

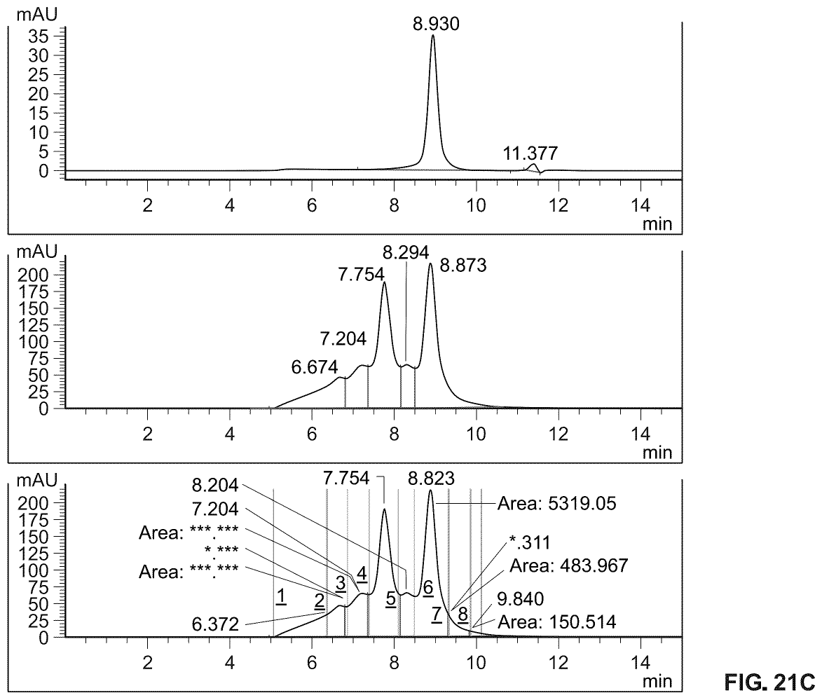

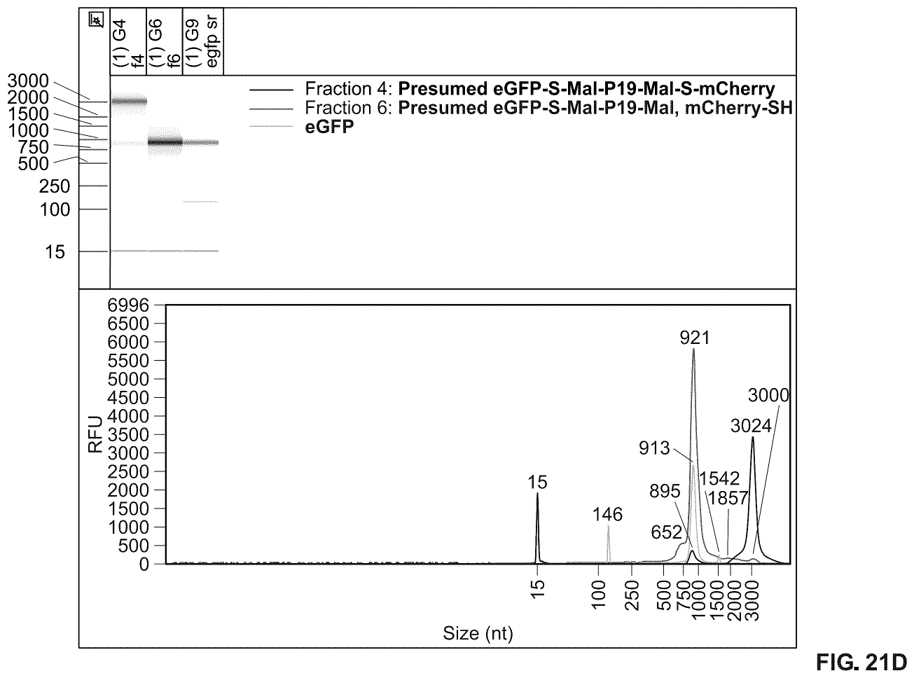

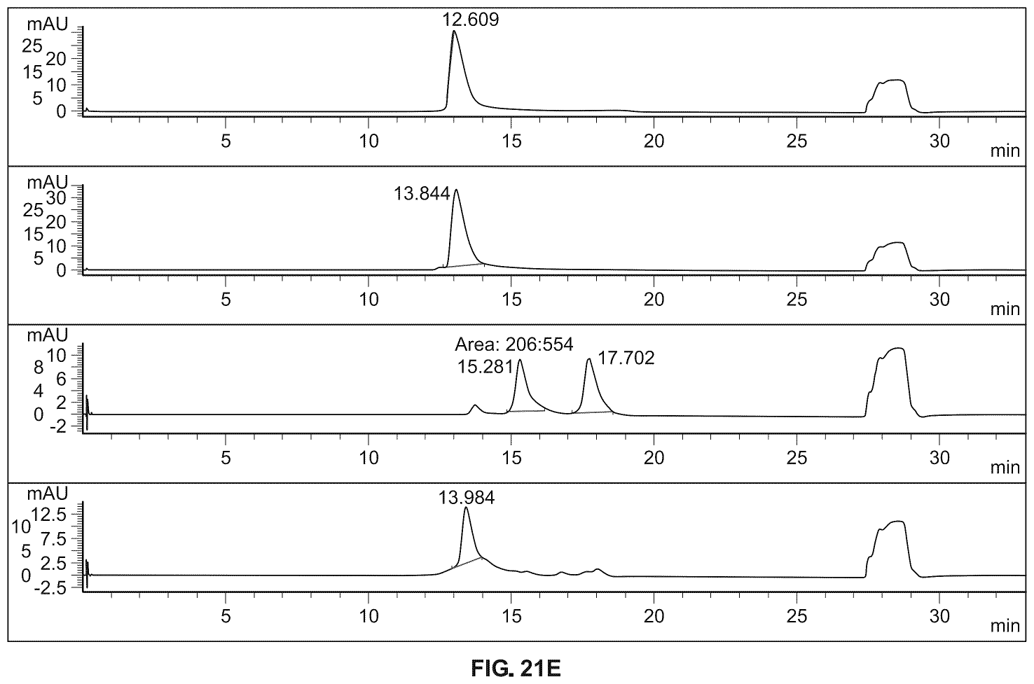

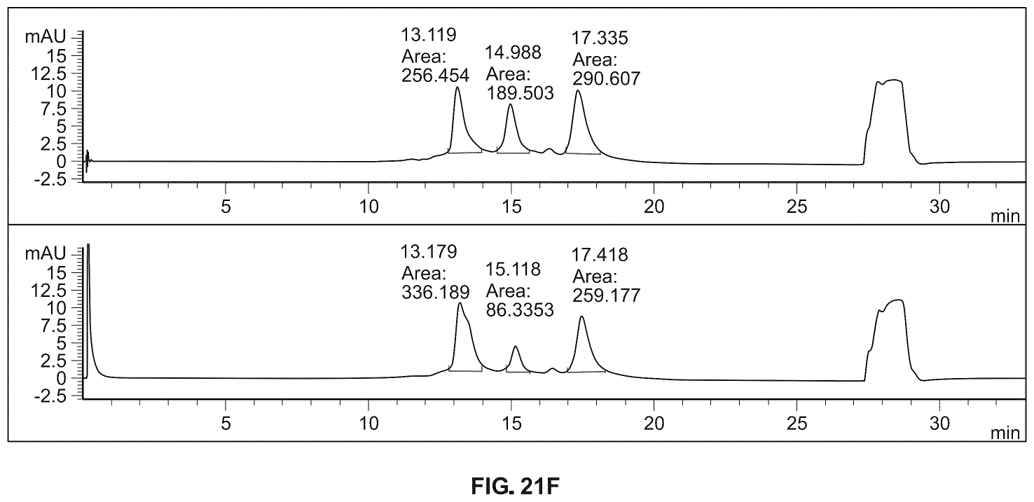

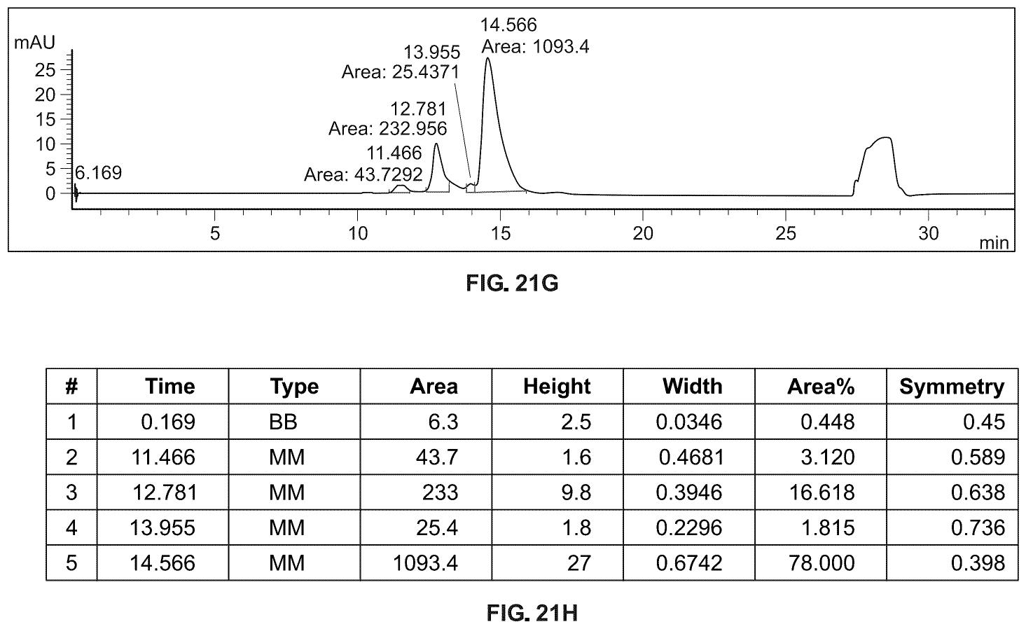

In a second method, mCherry-S-Maleimide-PEG19-Maleimide is treated with eGFP-SH (FIG. 21A). Briefly, mCherry-S-Maleimide-PEG19-Maleimide (60 μL, 64.9 ng/μL in Tris 1×, pH 7.0) is combined with eGFP-SH (SEQ ID NO: 95; 50 μL, 129.2 ng/μL) and the mixture is spin concentrated to a volume of ˜27.5 μL before it is incubated at room temperature for 4 hours. After incubation, the material is purified by RP-HPLC. Fractions containing the latter three peaks are buffer exchanged into citric acid buffer (50 mM, pH 5.5) to yield a solution containing eGFP-S-Maleimide-PEG19-Maleimide-S-mCherry (30 μL, 22.0 ng/μL). This reaction is monitored by HPLC with the parameters listed in Table 3. FIG. 21B shows the UV absorption chromatogram of the crude reaction mixture. Peaks at 8.800 min and 9.060 min correspond to eGFP-SH and peaks at 10.006 min and 10.373 min correspond to mCherry-S-Maleimide-PEG19-Maleimide. The right three peaks are collected for use in subsequent assays.

In a third method, GFP-S-Maleimide-PEG19-Maleimide is treated with mCherry-SH (FIG. 21A). Briefly, eGFP-S-Maleimide-PEG19-Maleimide (87 μL, 207 ng/μL, 5.30 μmol) and mCherry-SH (181 μL, 297 ng/μL, 15.9 μmol) are added sequentially to a 0.5 mL Eppendorf tube, and the solution is mixed by pipetting up and down 10 times. The mixture is incubated at room temperature for 2.5 h and subsequently at 4° C. for 14 h. After this time, the material is purified by size exclusion chromatography HPLC (SEC-HPLC) and fractions containing the presumed heterodimer and presumed monomer mixture are separately concentrated. The total recovery of the mixture containing eGFP-S-Maleimide-PEG19-S-mCherry is 75 μL, 37 ng/μL, and the recovery of the mixture containing eGFP-S-Maleimide-PEG19-Maleimide and mCherry-SH is 95 μL, 33 ng/μL.

The reaction of the third method is analyzed by SEC-HPLC, as shown in FIG. 21C. Briefly, the SEC-HPLC analysis uses an Agilent AdvanceBio SEC 1000 Å, 2.7 μm, 7.8×300 mm column. Part No. PL1180-5302. An isochratic method utilizing a flow rate of 1.0 mL/min is used with the mobile phase listed with a runtime of 15 min and is carried out at ambient temperature. The mobile phase is 20 mM Tris, 150 mM NaCl in water at a pH of 7.0. The SEC-HPLC chromatogram at an absorbance wavelength of 260 nm of eGFP only is shown in the top panel of FIG. 21C. The crude reaction mixture containing eGFP-S-Maleimide-PEG19-Maleimide-S-mCherry by the third method is shown in the middle panel of FIG. 21C. In the bottom panel of FIG. 21C, the same chromatogram is depicted with demarcated fractions from which eGFP-S-Maleimide-PEG19-Maleimide-S-mCherry is presumed to be within fraction 4, and the mixture containing eGFP-S-Maleimide-PEG19-Maleimide and mCherry-SH is presumed to be within Fraction 6. SEC Method 1000 Å.

Fractions 4 and 6 are isolated and capillary electrophoresis is performed on these fractions. As shown in FIG. 21D, Fraction 6 corresponds well with the size of eGFP, providing evidence for the presumed identity as a mixture of eGFP-S-Maleimide-PEG19-Maleimide and mCherry-SH. Fraction 4 contains little material that corresponds with the size of eGFP, and is, instead, more than double the size, providing evidence for the presumed identity as eGFP-S-Maleimide-PEG19-Maleimide-S-mCherry.

FIG. 21E shows RP-HPLC chromatograms of eGFP, mCherry, Fraction 4, and Fraction 6 respectively from top to bottom. The Fraction 6 chromatogram provides strong evidence for the presumed identity as primarily mCherry-SH since the peak expected for eGFP-S-Maleimide-PEG19-Maleimide (˜17.3 minutes) is minor. This suggests that the majority of the eGFP-S-Maleimide-PEG19-Maleimide is consumed during the reaction. On the other hand, the Fraction 4 chromatogram contains two peaks, with the one at 17.70 minutes being presumed to be eGFP-S-Maleimide-PEG19-Maleimide-S-mCherry due to the large retention time shift from eGFP and mCherry. The other significant peak at 15.3 minutes is likely to be mCherry disulfide (described in the section on the preparation of mCherry-SH) on account of the similar retention time and long reaction time (16 hours total).

The top panel of FIG. 21F shows RP-HPLC chromatogram at an absorbance wavelength of 260 nm of the crude reaction mixture containing eGFP-S-Maleimide-PEG19-Maleimide-S-mCherry (17.34 minutes) generated using the third method. This mixture is treated with DTT and then directly subjected to analysis as shown in the bottom panel of FIG. 21F. The peak tentatively assigned as the mCherry-SH disulfide homodimer (14.99 and 15.12 minutes for the top and bottom chromatograms, respectively) is significantly reduced while the peak tentatively assigned as eGFP-S-Maleimide-PEG19-Maleimide-S-mCherry (17.34 and 17.42 minutes for the top and bottom chromatograms, respectively) remains largely unchanged. This is consistent with the presumed disulfide being reduced into its monomeric form.

RP-HPLC chromatogram at an absorbance wavelength of 260 nm of another SEC-purified sample of eGFP-S-Maleimide-PEG19-Maleimide-S-mCherry is generated using the third method (FIG. 21G). In this case, the reaction only proceeded for 30 minutes before isolation, resulting in a greater ratio of the presumed product. Depicted below is the integrated spectrum, thus the purity of eGFP-S-Maleimide-PEG19-Maleimide-S-mCherry is assessed to be 78% based on area under the curve (FIG. 21H).

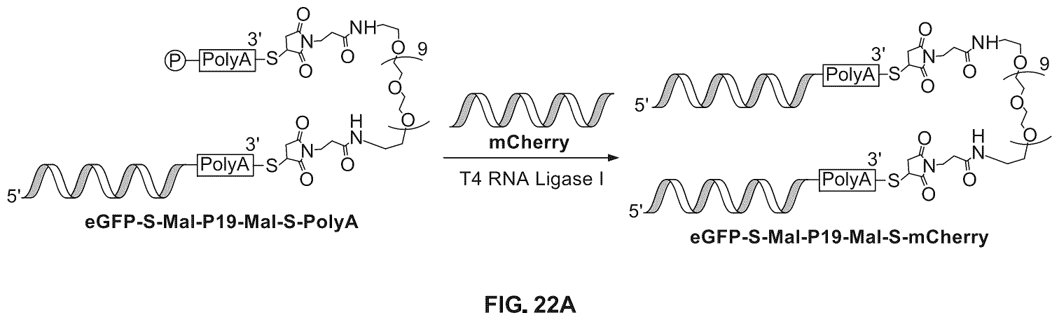

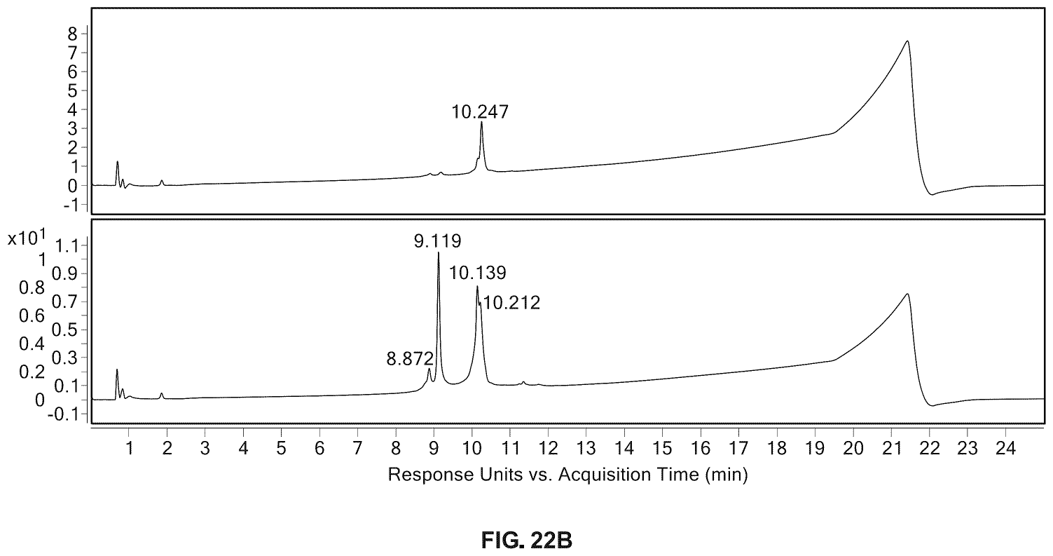

In a fourth method, mCherry is enzymatically ligated to eGFP-S-Maleimide-PEG19-Maleimide-S-PolyA (FIG. 22A). Briefly, the following are added to a 0.5 mL Eppendorf tube containing eGFP-S-Maleimide-PEG19-Maleimide-S-PolyA (SEQ ID NO: 93; 23 μL, 29.8 ng/μL, 685 ng in Tris 1× Buffer, pH 7.0):
(i) mCherry (3.5 μL, 3.5 μg)
(ii) 10× ligation buffer (7.0 μL)
(iii) Protector RNA Protector RNase Inhibitor (0.5 μL)
(iv) ATP (1.0 μL, 100 mM solution)
(v) 50% PEG-8000 (28 μL)
(vi) T4 RNA Ligase I (7.0 μL, high concentration, 30,000 units/mL)

The solution is mixed after each addition. The mixture is incubated for 6.5 hours and then purified using AMPure XP magnetic beads according to the manufacturer's protocol. After elution, the resulting crude product containing eGFP-S-Maleimide-PEG19-Maleimide-S-mCherry is obtained as a solution in water. The solution is measured at a concentration of 118.5 ng/μL with a total recovery volume of 28 μL. This reaction is monitored by HPLC with the parameters listed in Table 3. FIG. 22B shows the UV absorption chromatogram of the starting material eGFP-S-Maleimide-PEG19-Maleimide-S-PolyA (top) and the reaction mixture (bottom). Peaks at 8.872 min and 9.119 min correspond to mCherry.

### Time-Dependent Oligonucleotide Maleimide Hydrolysis

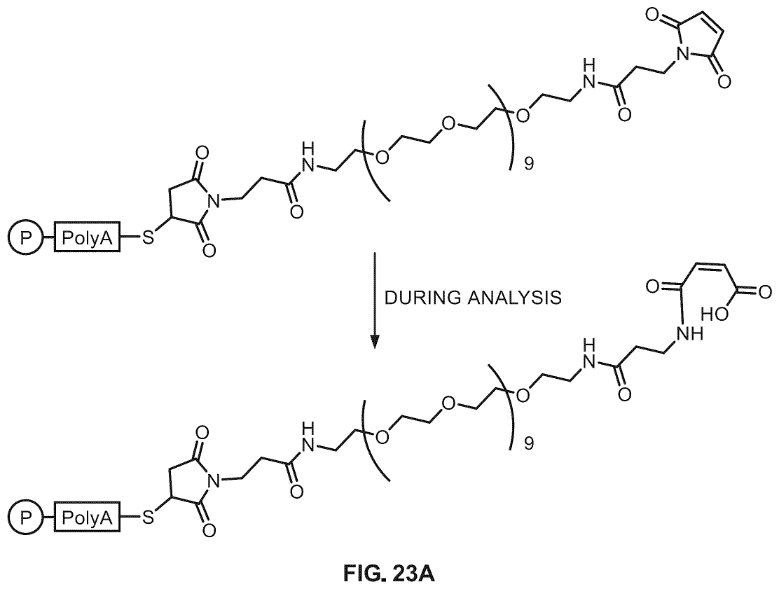

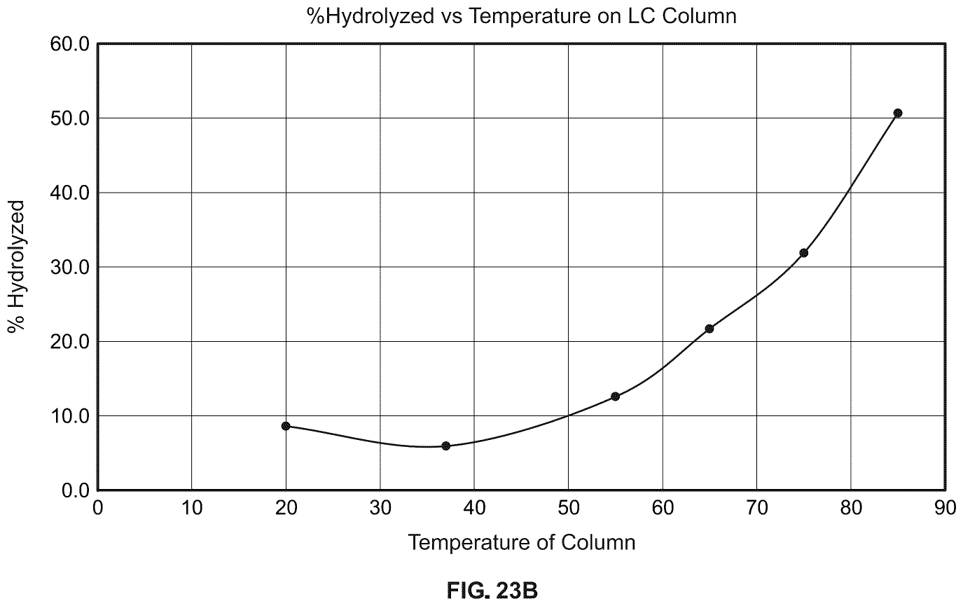

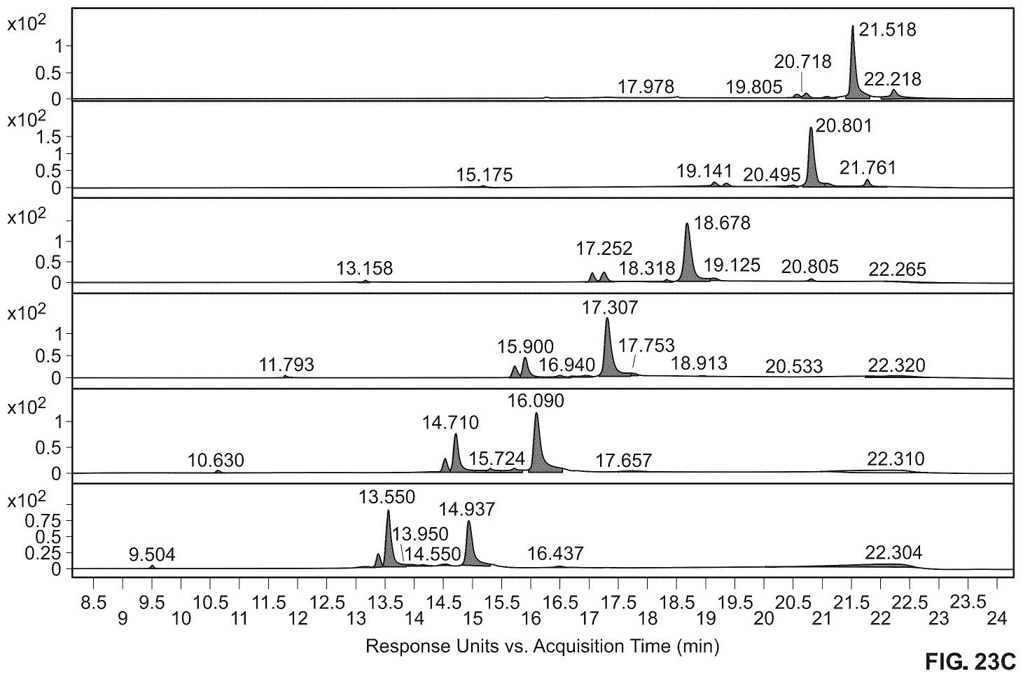

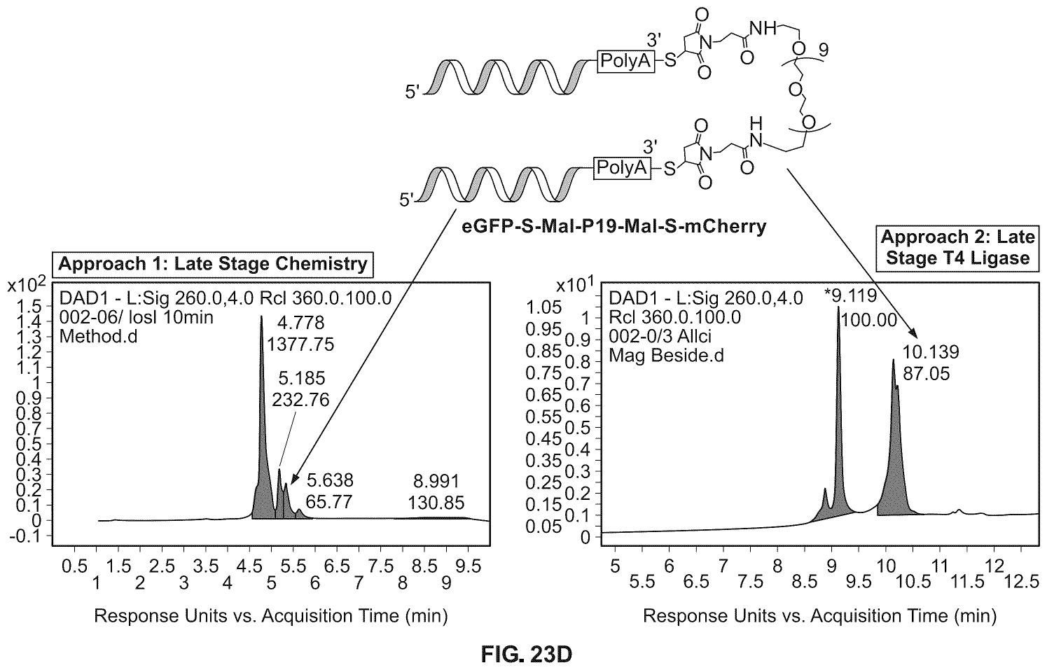

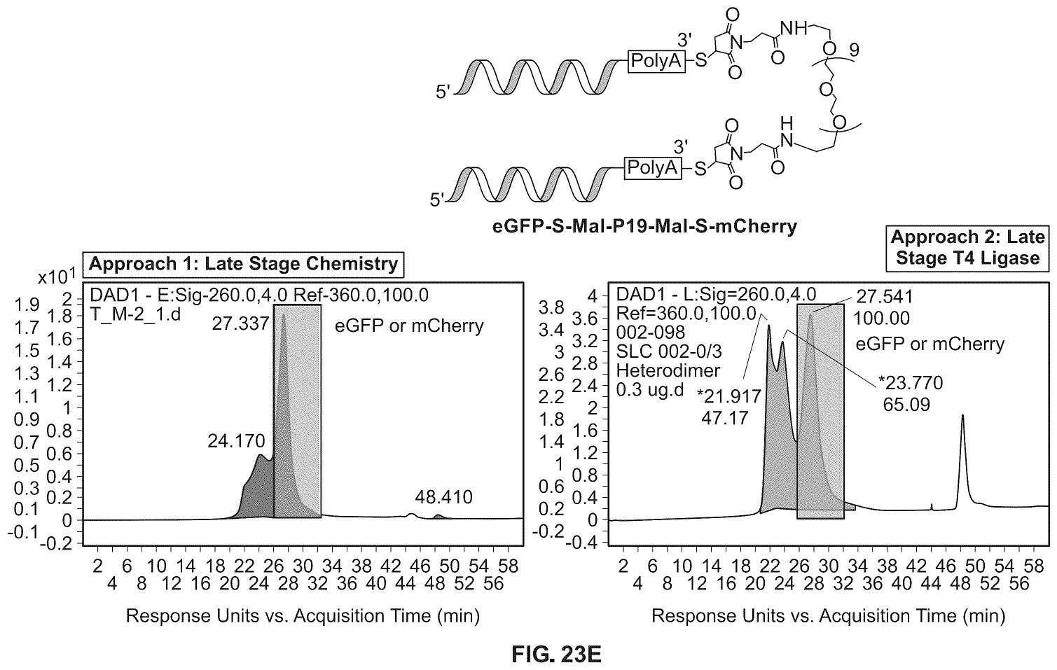

A study is conducted on PolyA-S-Maleimide-PEG19-Maleimide at various HPLC column temperatures. PolyA-S-Maleimide-PEG19-Maleimide is run on the RP-HPLC with the parameters as shown in Table 3, but at temperatures of 20° C., 37° C., 55° C., 65° C., 75° C., and 85° C. FIG. 23C shows the RP-HPLC chromatogram starting from 20° C. at the top through 37° C., 55° C., 65° C., 75° C., and finally to 85° C. at the bottom. It appears that during analysis on the RP-HPLC, maleimide hydrolyzes over time (FIG. 23A) and the amount of the +H₂O adduct increases with increasing column temperature (FIG. 23B). By extrapolation, it is estimated that prior to RP-HPLC analysis, up to 10% of the material exists in the hydrolyzed form of the compound (FIG. 23A). It is inferred that mRNA-S-Maleimide-PEG19-Maleimide compounds undergo a similar partitioning between the hydrolyzed and unhydrolyzed forms during RP-HPLC analysis at higher temperatures.

Due to the differences in the instability of the starting product, when comparing eGFP-S-Maleimide-PEG19-Maleimide-S-mCherry synthesized by chemical conjugation or enzymatic ligation (late stage T4 RNA Ligase), the distribution of products synthesized by enzymatic ligation appears to be different as shown by the larger fraction of unhydrolyzed product (FIG. 23D) and the sizing by size exclusion chromatography (FIG. 23E).

### Sortase Linkage

The Staphylococcus aureus sortase A Heptamutant (P94R, D160N, D165 Å, Y187L, E189R, K190E, K196T; GenBank Accession No. AF162687) is expressed and purified from an E. coli expression system.

The oligonucleotide bioconjugation method described herein is used to attach a peptide with sequence LPETGEK to the 3′end of a first mRNA (mRNA1). The oligonucleotide bioconjugation method described herein is also used to attach a peptide with sequence GGGG to the 3′end of a second mRNA (mRNA2). The two oligonucleotide bioconjugates mRNA1 and mRNA2 are mixed in equimolar quantities of 1 mM each, in 300 mM Tris pH 7.5 buffer containing 150 mM NaCl, 5 mM CaCl₂, and 5% v/v DMSO. The enzyme is added to the mRNA1 and mRNA2 mixture to a final concentration of 50 nM. The reaction is incubated at 22.5° C. for 20 min. The reaction may be quenched by adding 0.5 volumes of 1 M HCl. The reaction product is assayed by RP-HPLC using the parameters provided in any one of Tables 1-4 and 6.

Enzymatic ligation of mRNA with Modified Single Nucleotide and Subsequent Conjugation

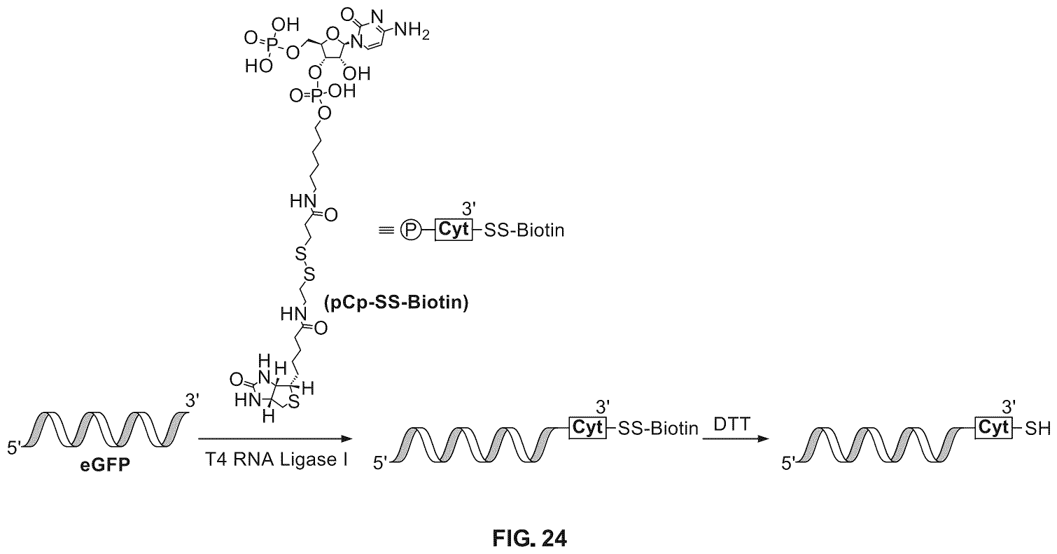

The 3′ end of an mRNA can also be modified with a single nucleotide to allow for subsequent conjugation (FIG. 24). Briefly, eGFP (50.0 μL, 1.0 mg/mL in 1.0 mM Citric Acid, pH 6.5), pCp-SS-Biotin (3.13 μL, 1.0 mM solution in water, from Jena Biosciences and used as received), 10× T4 RNA Ligase Reaction Buffer (15.6 μL), ATP (3.9 μL, 100 mM solution in water), Protector RNase Inhibitor (5.0 μL, obtained from Millipore-Sigma, Catalogue 3335399001 and used as received), water (70.8 μL), and T4 RNA Ligase I (7.8 μL, 30000 units/mL), are sequentially added and mixed by pipetting up and down 20 times. The mixture is incubated at room temperature (approximately 16° C.) for 2 h 10 min. After this time, an aliquot (1.0 μL) of the reaction mixture is diluted with 20 μL of 7.5% acetonitrile in water containing 50 mM TEAA (triethylammonium acetate) at a pH of 7.0 (Solution A in Table 7), and 20 μL of this resulting solution was analyzed by RP-HPLC using the parameters provided in Table 7. Solvent A was comprised of 7.5% acetonitrile in water containing 50 mM TEAA (triethylammonium acetate) at a pH of 7.0. Solvent B was comprised of a 18% acetonitrile in water containing 50 mM TEAA at a pH of 7.0. Solvent C was comprised of 75% acetonitrile in water containing 50 mM TEAA. The 10× buffer, ATP, and T4 RNA Ligase I are obtained from NEB kit Cat. No. M0437M and used as received.

TABLE 7
RP-HPLC and MS parameters for monitoring
synthesis of eGFP-S-Maleimide-SulfoCy5.

| | |
|---|---|
| Column | Sartorius ClMac ™ SDVB 0.1 mL Analytical |
| | Column, 2 μm |
| Column Temperature | 60° C. |
| Column flow rate | 1.0 mL/min |
| Solvent A | 7.5% acetonitrile in water containing 50 mM |
| | TEAA (triethylammonium acetate) at a pH of 7.0 |
| Solvent B | 18% acetonitrile in water containing 50 mM |
| | TEAA at a pH of 7.0 |
| Solvent C | 75% acetonitrile in water containing 50 mM |
| | TEAA at a pH of 7.0 |
| Gradient | 0.0-0.2 min 100% A, 0% B, 0% C |
| | 0.2-26.45 min 0% A, 100% B, 0% C |
| | 26.45-26.65 min 0% A, 0% B, 100% C |
| | 26.65-26.86 min 0% A, 0% B, 100% C |
| | 27.86-28.00 min 100% A, 0% B, 0% C |
| | 28.00-33.00 min 100% A, 0% B, 0% C |

Conjugation of Peptides to mRNA

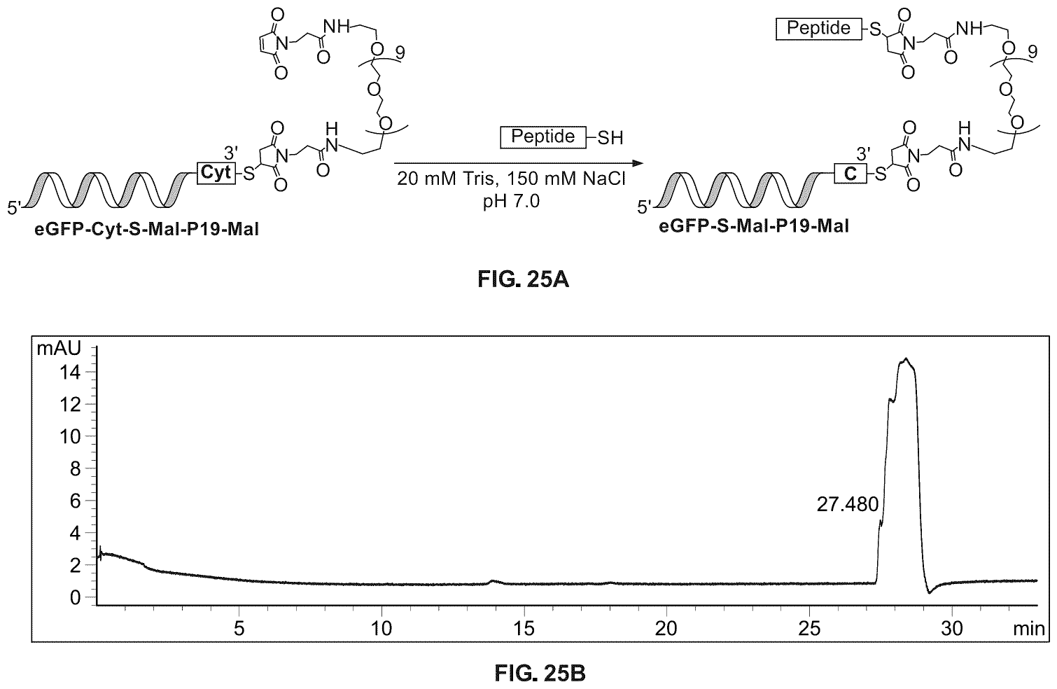

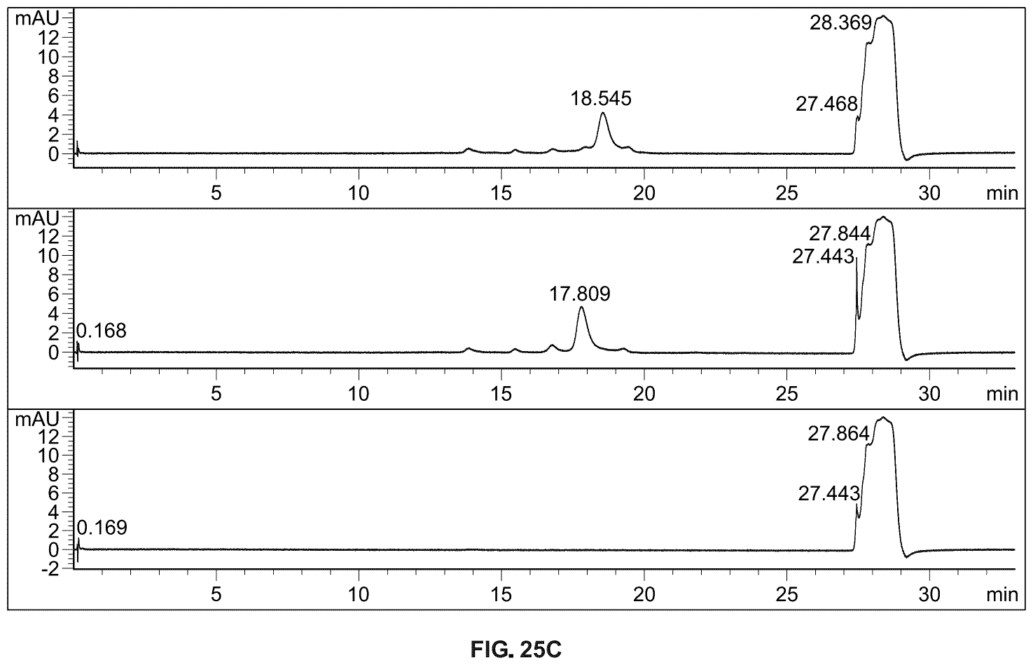

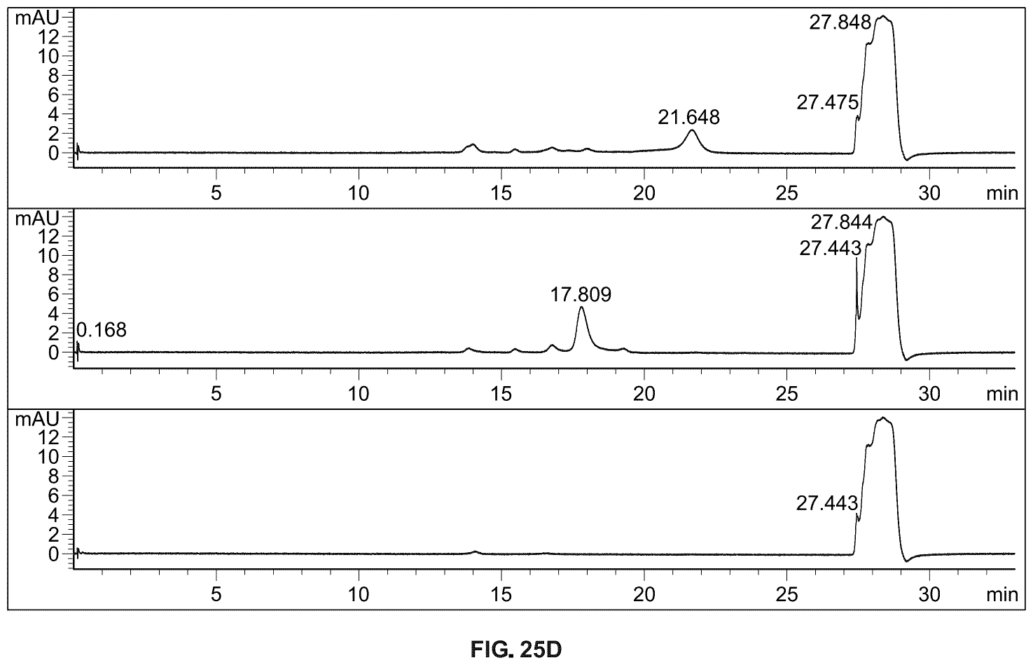

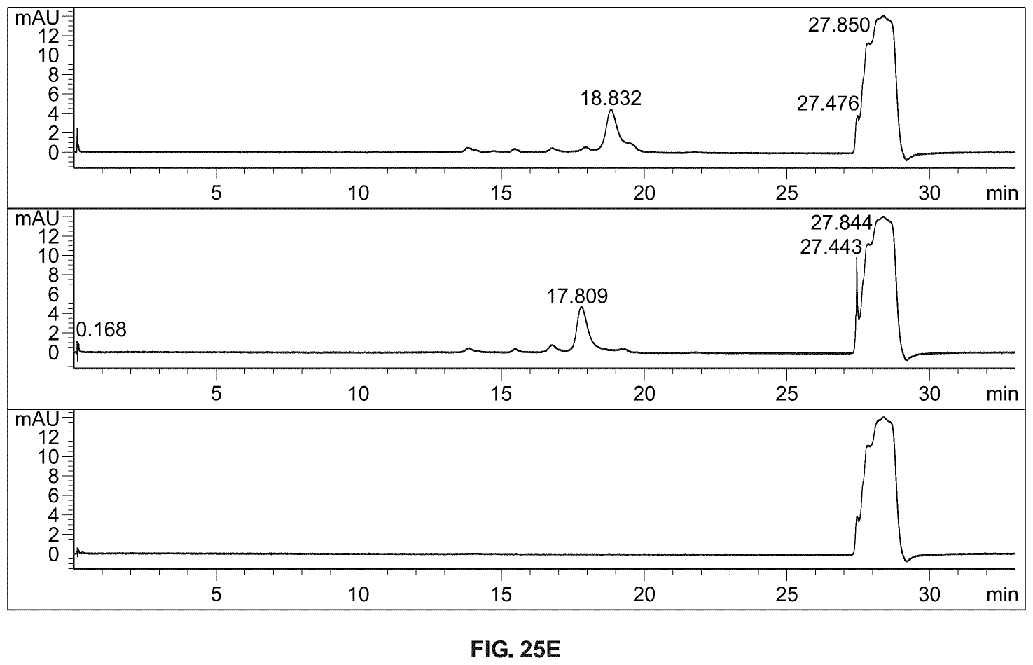

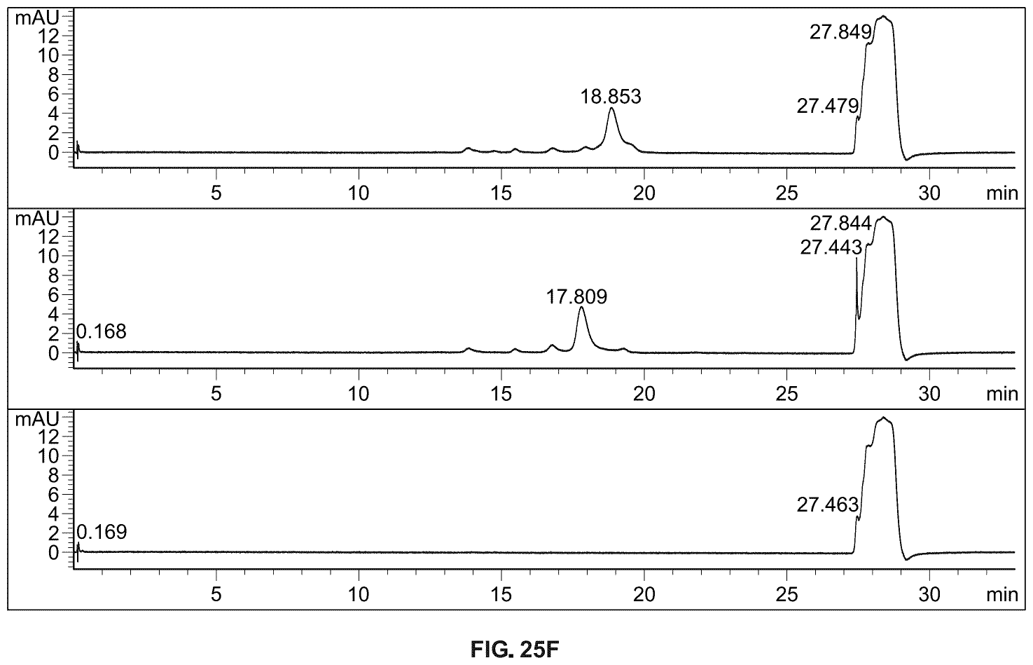

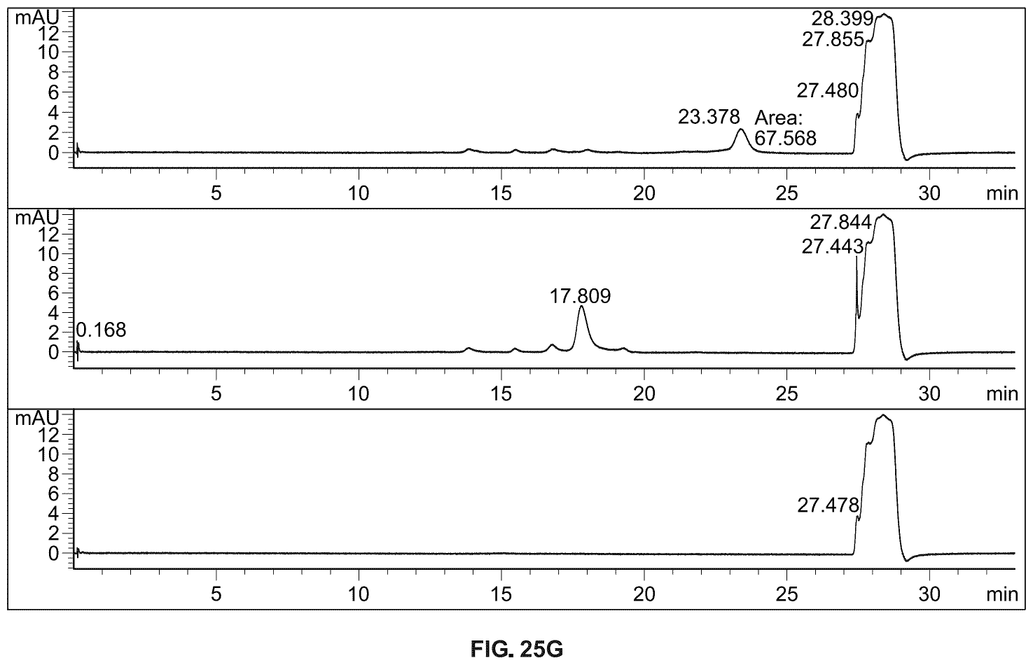

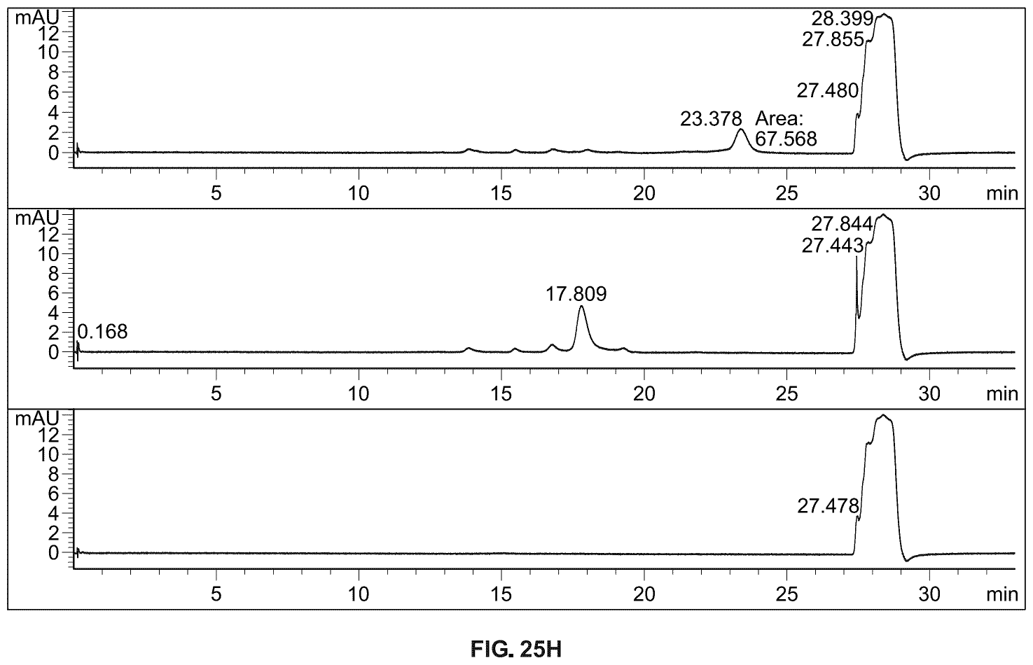

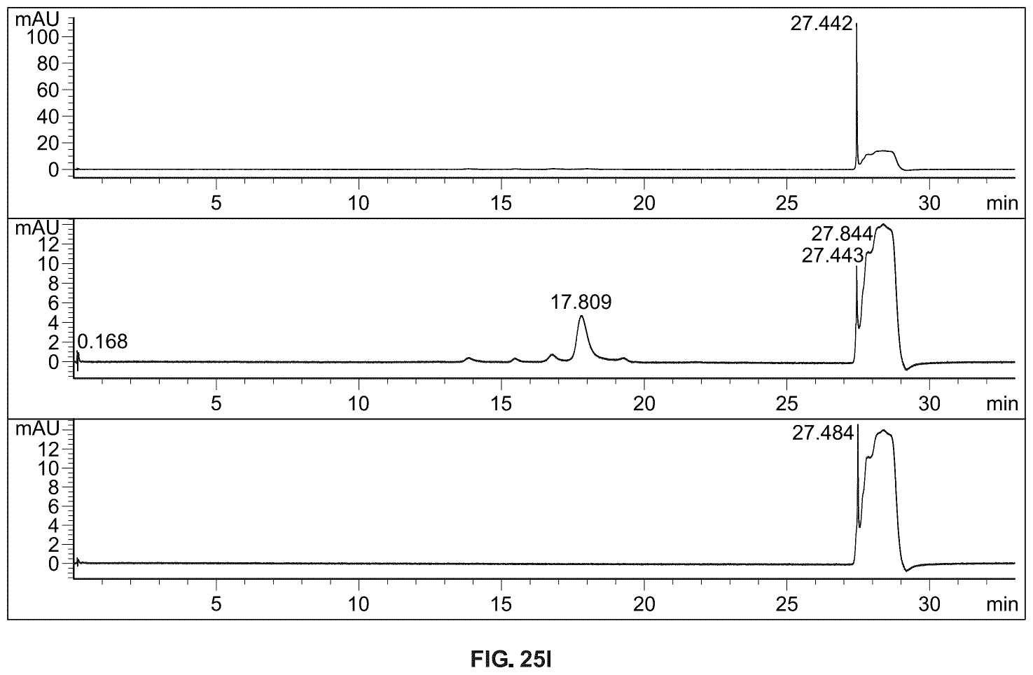

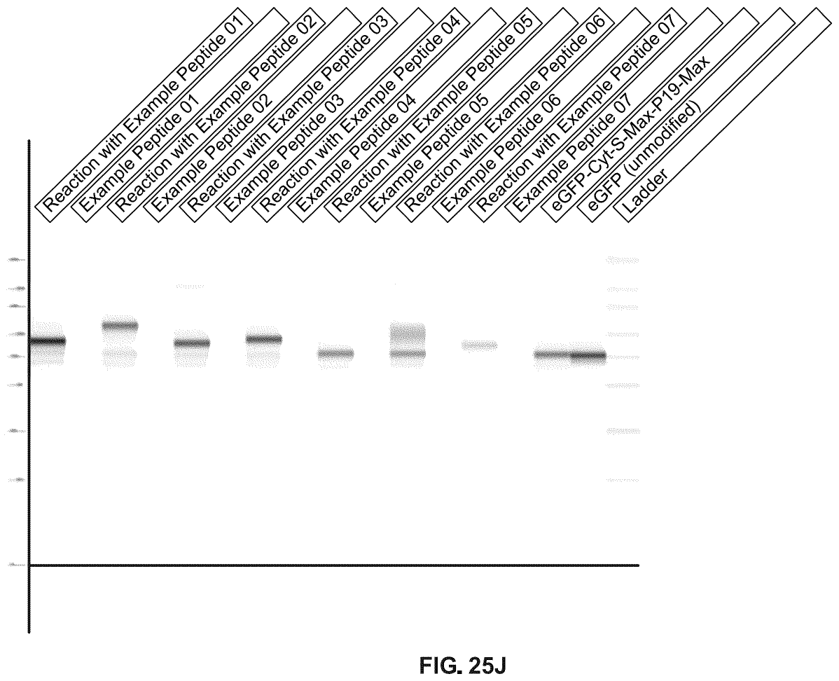

Peptides and mRNA may be conjugated using a strategy shown in FIG. 25A. Briefly, a solution of peptide (1.0 μL, 200 μM solution in water) is added to a solution containing eGFP-Cyt-S-Maleimide-PEG19-Maleimide (SEQ ID NO: 99; 9.0 μL, 66.5 ng/μL in 20 mM in Tris, 150 mM in NaCl at a pH of 7.0) in a PCR tube or Eppendorf tube. The final concentration of eGFP-Cyt-S-Maleimide-PEG19-Maleimide is approximately 60 ng/uL with a final peptide concentration of 20 μM. The solution is allowed to incubate for 30 min.

All conjugation reactions are analyzed by RP-HPLC using the parameters shown in Table 7, by diluting 3.0 μL of the reaction mixture prepared as above in 20.0 μL of 7.5% acetonitrile in water containing 50 mM TEAA (Solvent A), and injecting 20.0 μL for analysis. Alternatively, reactions were analyzed by Fragment Analyzer according to manufacturer instructions.

A number of exemplary peptides were tested. As shown by the UV chromatogram of a blank run in FIG. 25B, peaks from ˜27.3-29.2 min coincide with column wash (100% Solvent C). FIG. 25C shows the UV chromatograms when Peptide 1 (SEQ ID NO: 101; PASPASPASPASPASC-NH₂) is reacted with eGFP-Cyt-S-Maleimide-PEG19-Maleimide (top), eGFP-Cyt-S-Maleimide-PEG19-Maleimide is run alone (middle), and Peptide 1 is run alone (bottom).

FIG. 25D shows the UV chromatograms when Peptide 2 (SEQ ID NO: 102; PASPASPASPASPASPASPASPASPASPASPASPASPASPASPASC-NH₂) is reacted with eGFP-Cyt-S-Maleimide-PEG19-Maleimide (top), eGFP-Cyt-S-Maleimide-PEG19-Maleimide is run alone (middle), and Peptide 2 is run alone (bottom).

FIG. 25E shows the UV chromatograms when Peptide 3 (SEQ ID NO: 103; CPASPASPASPASPASKDEL-NH₂) is reacted with eGFP-Cyt-S-Maleimide-PEG19-Maleimide (top), eGFP-Cyt-S-Maleimide-PEG19-Maleimide is run alone (middle), and Peptide 3 is run alone (bottom).

FIG. 25F shows the UV chromatograms when Peptide 4 (SEQ ID NO: 104; CPASPASPASPASPASDEKKMP-NH₂) is reacted with eGFP-Cyt-S-Maleimide-PEG19-Maleimide (top), eGFP-Cyt-S-Maleimide-PEG19-Maleimide is run alone (middle), and Peptide 4 is run alone (bottom).

FIG. 25G shows the UV chromatograms when Peptide 5 (SEQ ID NO: 105; RGVPHIVMVDAYKRYKSGGSC-NH₂) is reacted with eGFP-Cyt-S-Maleimide-PEG19-Maleimide (top), eGFP-Cyt-S-Maleimide-PEG19-Maleimide is run alone (middle), and Peptide 5 is run alone (bottom).

FIG. 25H shows the UV chromatograms when Peptide 6 (SEQ ID NO: 106; VTTLSGLSGEQGPSGDMTTEEDSATHIKFSKRDEDGRELAGATMELRDCSGKTISTWISD GHVKDFYLYPGKYTFVETAAPDGYEVATPIEFTVNEDGQVTVDGEATEGDAHT-NH₂) is reacted with eGFP-Cyt-S-Maleimide-PEG19-Maleimide (top), eGFP-Cyt-S-Maleimide-PEG19-Maleimide is run alone (middle), and Peptide 6 is run alone (bottom). Initial preparation of a 1.0 mM stock solution of Peptide 6 was Peptide 6 diluted in 50:50 water:DMSO followed by dilution with water to a 200 μM stock solution.

FIG. 25I shows the UV chromatograms when Peptide 7 (SEQ ID NO: 107; Y-Aib-EGTFTSDYSI-Aib-LDKIAQK(GGC)AFVQWLIAGGPSSGAPPPS-NH₂) is reacted with eGFP-Cyt-S-Maleimide-PEG19-Maleimide (top), eGFP-Cyt-S-Maleimide-PEG19-Maleimide is run alone (middle), and Peptide 7 is run alone (bottom).

Each of the reactions of Peptides 1-7 are analyzed using capillary electrophoresis (CE). FIG. 25J shows CE data is obtained using an Agilent Fragment Analyzer 5200 fitted with a FA 12-capillary array short, 33 cm (Product ID A2300-1250-3355) utilizing an Agilent HS RNA Kit (15 NT) (Product ID DNF-472-0500) according to the manufacturer's instructions. Samples are prepared by the dilution of an aliquot of sample into a range of 0.1-1.0 ng/mL using the included diluent in the kit and melted at 70° C. for 2 min followed by immediate cooling to 4° C. for a minimum of one minute in a PCR thermocycler. Each lane of FIG. 25J shows each reaction mixture followed immediately by a control of analysis of the peptide only on the CE system. Largely, the peptides alone give no signal in the CE method.

Isolation of an mRNA-Peptide Conjugate

The method for isolating the mRNA-peptide bioconjugate is disclosed here. Briefly, eGFP-Cyt-S-Maleimide-PEG19-Maleimide (30 μL, 146.7 ng/μL in water), Tris Buffer (4.0 μL, 200 mM Tris, 1.5 M NaCl, pH 7.0), and Peptide 1 (3.0 μL, 200 μM in water) are added sequentially to a 0.5 mL Eppendorf tube. The mixture is incubated for approximately 30 minutes followed by isolation of the mRNA via a Monarch® Spin RNA Cleanup kit (50 μg) using 80 μL of 6 M Guanidine-HCl for a binding buffer and 132 μL of ethanol. The Spin column is washed twice with 500 μL of wash buffer and finally the mRNA is eluted with 25 μL of water, then a second elution with 20 μL of water. Both eluants are combined to give a total of ˜45 μL solution whose concentration is estimated by absorbance at 260 nm on a nanodrop system. Ultimately, 45 μL of eGFP-Cyt-S-Maleimide-PEG19-Maleimide-Peptide 1 at 71.2 ng/μL is recovered. About 7.5 μL of this material with diluted with 20 μL of 7.5% acetonitrile in water containing 50 mM TEAA (Solution A in Table 7), and 20 μL is injected for RP-HPLC analysis.

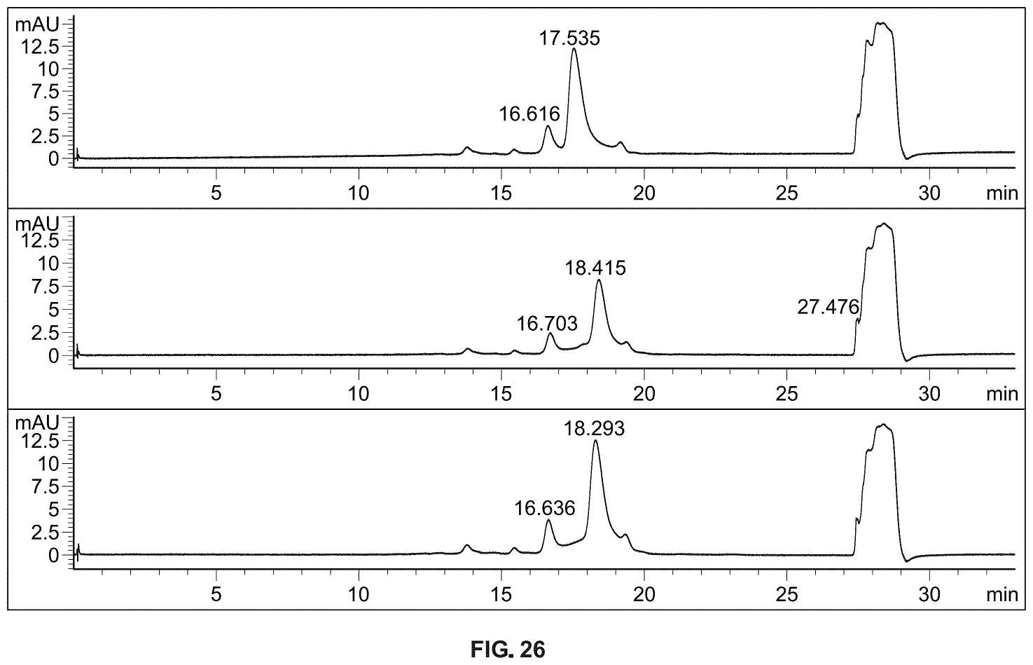

The UV chromatograms are shown in FIG. 26. As shown in FIG. 26 (top), the peak at 17.5 min is likely to be unconjugated unhydrolyzed eGFP-Cyt-S-Maleimide-PEG19-Mal. The hydrolyzed eGFP-Cyt-S-Maleimide-PEG19-Maleimide is forms a peak at 16.6-16.7 min, and is less efficient in conjugation with the thiol containing peptide. The middle trace of FIG. 26 depicts the reaction of eGFP-Cyt-S-Maleimide-PEG19-Maleimide with Peptide 1 after 10 minutes. Finally, the bottom trace of FIG. 26 shows the resultant material after Monarch Spin Cleanup kit using the conditions specified of the reaction of eGFP-Cyt-S-Maleimide-PEG19-Maleimide with Peptide 1. Chromatograms shown are at an absorbance of 260 nm.

### Example 2: In Vitro Biological Data for Oligonucleotide Bioconjugates

Experiment: Flow Cytometry on Cells Transfected with eGFP-S-Maleimide-SulfoCy5

HEK-293T cells are cultured in Dulbecco's modified Eagle's medium (DMEM, high glucose, 110 mg/L pyruvate, Thermo Fisher Scientific GlutaMAX) supplemented with 10% fetal bovine serum (FBS, Thermo Fisher Scientific) and 1% penicillin-streptomycin (Thermo Fisher Scientific) at 37° C. in a 5% CO2 humidified incubator. Cells are seeded into 96-well plates (Fisher Scientific, flat bottom) at 30,000 cells/well in 150 μL media 18 hours before transfection. Wells on the edge of the plate are not seeded but are instead filled with 150 μL Dulbecco's phosphate buffered saline (DPBS, Corning). Standard sterile technique is used in a biosafety cabinet when handling tissue cultures to avoid contamination/destruction of the cell culture during manipulations.

Cells are transfected with eGFP mRNA, eGFP-S-Maleimide-SulfoCy5 mRNA bioconjugate, or transfection reagent (control) in three ways: (i) Lipofectamine 3000, (ii) Lipofectamine MessengerMAX, and (iii) lipid nanoparticles.

In the first method, Lipofectamine 3000 (Thermo Fisher Scientific) reagent is used according to the manufacturer's protocol. Volumes provided herein correspond with the amount of reagent and mRNA for a single well on a 96-well plate (Table 8). Briefly, 0.30 μL of the Lipofectamine 3000 reagent is diluted into 5.00 μL of minimal essential media (Opti-MEM, Thermo Fisher Scientific) in a 200 μL microcentrifuge tube. In a second microcentrifuge tube, the mRNA (5-400 ng at concentrations ranging 10-1000 ng/μL) and P3000 reagent (2 μL/pg mRNA) are diluted into 5.00 μL of Opti-MEM. The contents of the first microcentrifuge tube are added to the second microcentrifuge tube, mixed, and incubated at room temperature for 15 minutes. The mixture is then added directly to a well of plated cells. The final volumes are as follows: for the control group, cells are treated with buffer only (5.68 μL, 20 mM Tris, 150 mM NaCl, pH 7.6); for test groups, cells are treated with 100 ng of eGFP mRNA or eGFP-S-Maleimide-SulfoCy5 mRNA bioconjugate (5.68 μL, 17.6 ng/μL in 20 mM Tris, 150 mM NaCl, pH 7.6). All cells are treated in duplicates.

In the second method, Lipofectamine MessengerMAX (Thermo Fisher Scientific) reagent is used according to the manufacturer's protocol. Volumes provided herein correspond with the amount of reagent and mRNA for a single well on a 96-well plate (Table 8). Briefly, 0.30 μL of the Lipofectamine MessengerMAX reagent was diluted into 5.00 μL of minimal essential media (Opti-MEM, Thermo Fisher Scientific) in a 200 μL microcentrifuge tube and allowed to incubate for 10 minutes. In a second microcentrifuge tube, the mRNA (5-400 ng at concentrations ranging 10-1000 ng/μL) is diluted into 5.00 μL of Opti-MEM. The contents of the first tube added to the second tube, mixed, and allowed to incubate at room temperature for 5 minutes. The mixture is then added directly to a well of plated cells.

In the third method, solutions of mRNA and derivatized mRNA formulated in lipid nanoparticles (LNP) are used directly in treatment of adherent cells. Depending on the concentration of mRNA LNPs (200-1000 ng/μL), a volume of solution is directly dispensed into a well of plated cells.

TABLE 8
Transfection Reaction Conditions

| | Transfection | | |
|---|---|---|---|
| Treatment Group | Reagent | Payload | Buffer |
| Control | Lipofectamine | — | 5.68 μL, 20 mM |
| (Transfection | 3000 | | Tris, 150 mM |
| Reagent only) | | | NaCl, pH 7.6 |
| eGFP mRNA | Lipofectamine | 100 ng EGFP | 5.68 μL, 20 mM |
| | 3000 | mRNA | Tris, 150 mM |
| | | | NaCl, pH 7.6 |
| eGFP-S-Maleimide- | Lipofectamine | 100 ng eGFP- | 5.68 μL, 20 mM |
| SulfoCy5 mRNA | 3000 | S-Maleimide- | Tris, 150 mM |
| bioconjugate | | SulfoCy5 | NaCl, pH 7.6 |

After transfection, cells are allowed to incubate for 24 hours before being dissociated for flow cytometry measurements. Briefly, media is removed by aspiration followed by the addition of 75 μL of TrypLE Express Enzyme (TrypLE, Thermo Fisher Scientific). Once cell dissociation is complete, 75 μL of media is added to the well and the cells are gently mixed by pipette. The entire contents of the well are then transferred to a V-bottom 96-well plate (Millipore Sigma). The cells are then pelleted by centrifugation of the V-bottom plate (350 rcf, 5 minutes), the supernatant is discarded, and the cells are resuspended in 150 μL of DPBS. The cells are again pelleted (350 rcf, 5 minutes), the supernatant is discarded, and the pelleted cells are resuspended in 100 μL of DPBS before being subjected to flow cytometry analysis.

A BD Accur C₆ Plus Personal Flow Cytometer is used to analyze cells suspended in DPBS in V-Bottom well plates. Particles (primarily cells and cell debris) are recorded by the instrument as “events,” and for all analysis, at least 5000 events are recorded. The intake flowrate used in all cases is 66 μL/minute which results in a corresponding core size of 22 μm. BD CSampler™ Software is used for all analysis. A blue excitation laser with a 530/30 emission filter (green channel) is used to probe eGFP. A red excitation laser with a 675/25 filter (red channel) is used to probe eGFP-S-Maleimide-SulfoCy5.

### Results:

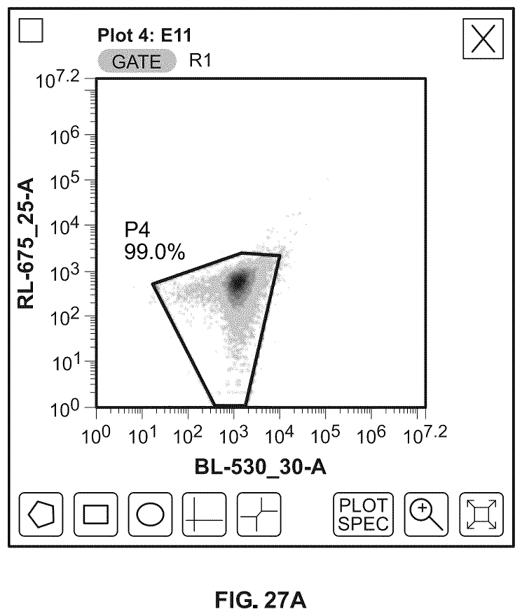

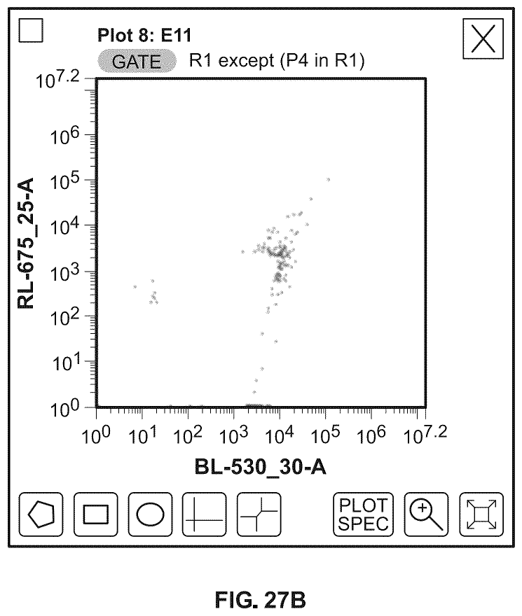

Representative flow cytometry plots are shown in FIG. 27. A majority of cells (>99%) show no significant fluorescence in either channel. FIG. 27A depicts the raw data including the majority of cells that are not fluorescent in either channel. FIG. 27B depicts the data after the cells that are not fluorescent in either channel are removed (P4 gating). The plot is further sub-divided into quadrants. The horizontal bisecting line is placed at a fluorescence intensity (675/25 filter) of 200 (a.u.) at which <0.5% of recorded events reached a fluorescence intensity above this value. The vertical bisecting line is placed at a fluorescence intensity of 1000 (a.u.) at which <0.5% of recorded events reached a fluorescence intensity above this value. These are the thresholds above which events are considered red (675/25) or green (530/30) fluorescence, respectively. As such, all events are considered as one of four outcomes depending on the quadrant they appear: (i) upper left (UL)—positive red (675/25) fluorescence; (ii) upper right (UR)—positive red (675/25) fluorescence and positive green (530/30) fluorescence; (iii) lower left (LL)—no fluorescence; and (iv) lower right (LR)—positive green (530/30) fluorescence.

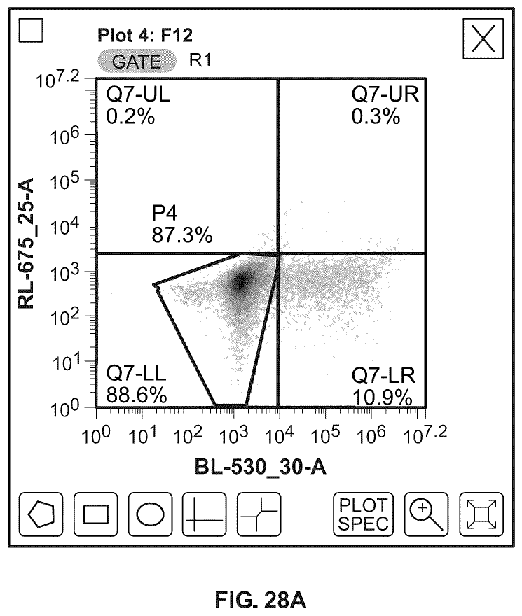

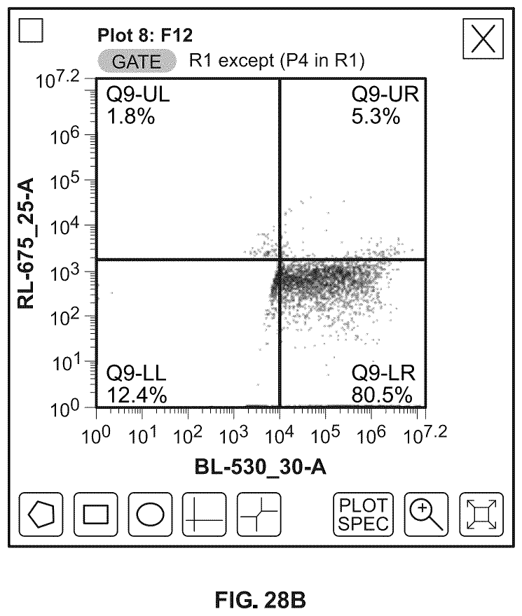

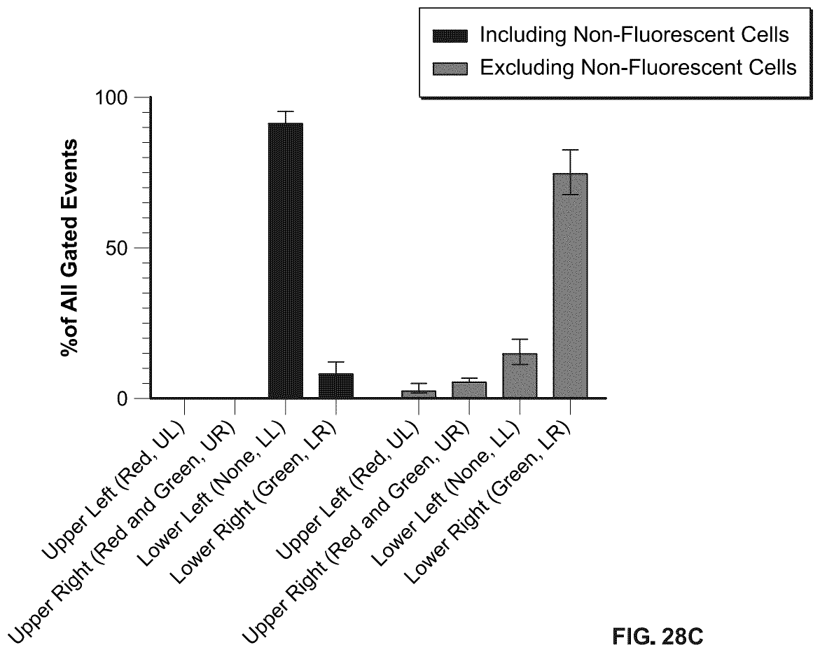

The gated flow cytometry data is shown in FIG. 28A (without P4 gate) and FIG. 28B (with P4 gate). For cells transfected with eGFP mRNA, while most cells (91% average) do not fluoresce in either channel, the majority of those that do fluoresce (75% average) produce fluorescence in the green channel only (FIG. 28B lower right quadrant). FIG. 28C depicts the tabulated duplicates in bar graph form to the right for cells treated with eGFP. The bar graph provides the average % of all gated events within a given quadrant for the duplicate runs along with a standard deviation (SD).

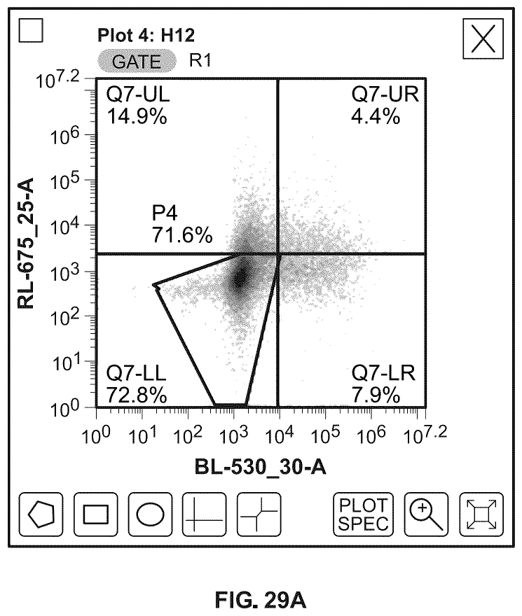

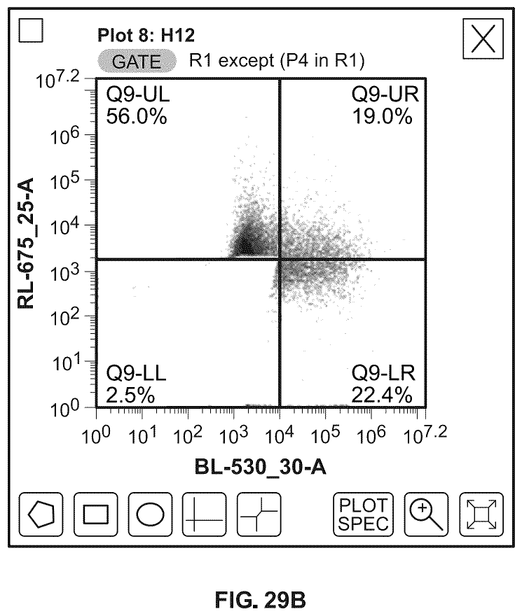

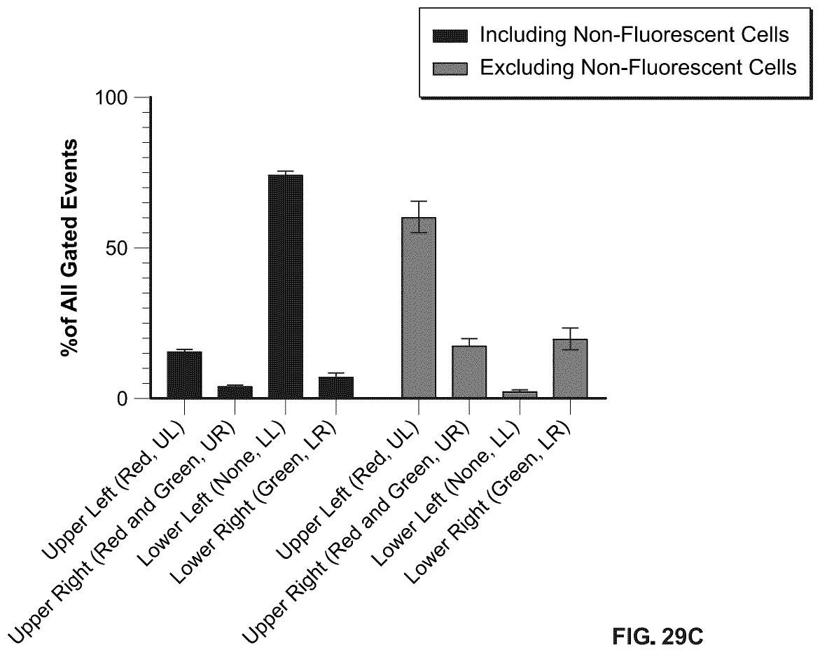

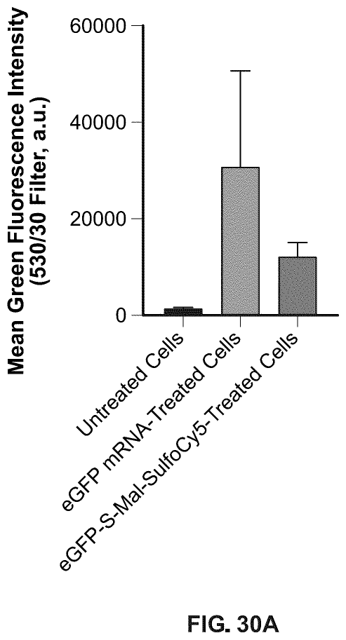

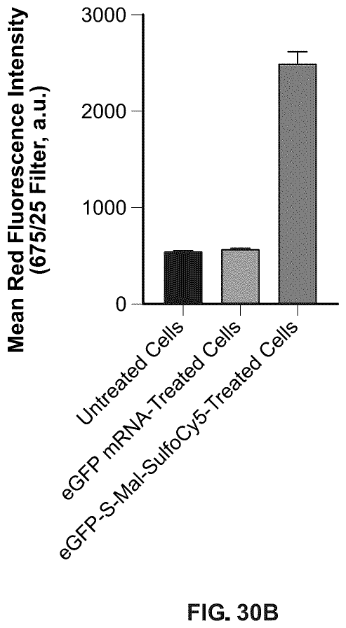

As shown in FIGS. 29A-C, for cells transfected with eGFP-S-Maleimide-SulfoCy5 mRNA bioconjugate, while most cells (73% average) do not fluoresce in either channel, for the cells that do fluoresce, 60% fluoresce in the red channel only (FIG. 29B upper left quadrant), 20% fluoresce in the green channel only (FIG. 29B lower right quadrant), and 18% fluoresce in both the green and the red channels (FIG. 29B upper right quadrant). FIGS. 30A and 30B provide the mean fluorescence intensity (a.u.) for the green (530/30 filter) and red (675/25 filter) channels, respectively, for the duplicate runs along with a SD. Thus, cells treated with eGFP-S-Maleimide-SulfoCy5 generate a significant fluorescence intensity in both the red and green channels (FIGS. 29C, 30A-B).

Experiment: Flow Cytometry on Cells Transfected with eGFP-S-Maleimide-PEG19-Maleimide-S-mCherry

HEK-293T cells are cultured in conditions identical to the eGFP-S-Maleimide-SulfoCy5 experiment above. Cells are transfected with eGFP mRNA, mCherry mRNA, and eGFP-S-Maleimide-PEG19-Maleimide-S-mCherry mRNA, or buffer (control), using Lipofectamine 3000 (Thermo Fisher Scientific) reagent according to the manufacturer's protocol, similar to the transfection protocol detailed above for the eGFP-S-Maleimide-SulfoCy5 experiment. For the control group, cells are treated with buffer only (4.67 μL, 50 mM Sodium Citrate, pH 5.5). For the treatment groups, cells are treated with 100 ng of eGFP mRNA (2.33 μL, 42.9 ng/μL in 50 mM Sodium Citrate, pH 5.5), 100 ng of mCherry mRNA (2.33 μL, 42.9 ng/μL in 50 mM Sodium Citrate, pH 5.5), or 200 ng of eGFP-S-Maleimide-PEG19-Maleimide-S-mCherry mRNA (4.67 μL, 42.9 ng/μL in 50 mM Sodium Citrate, pH 5.5). Since the bioconjugate eGFP-S-Maleimide-PEG19-Maleimide-mCherry is about twice the molecular weight of either eGFP or mCherry mRNA alone, cells are treated with twice the amount of bioconjugate to ensure approximately the same amount of each respective mRNA within the bioconjugate. All cells are treated in triplicates.

TABLE 9
Transfection Reaction Conditions

| | Transfection | | |
|---|---|---|---|
| Treatment Group | Reagent | Payload | Buffer |
| Control | — | — | 4.67 μL, 50 mM |
| (Buffer only) | | | Sodium Citrate, |
| | | | pH 5.5 |
| eGFP mRNA | Lipofectamine | 100 ng EGFP | 2.33 μL, 50 mM |
| | 3000 | mRNA | Sodium Citrate, |
| | | | pH 5.5 |
| mCherry mRNA | Lipofectamine | 100 ng | 2.33 μL, 50 mM |
| | 3000 | mCherry | Sodium Citrate, |
| | | mRNA | pH 5.5 |
| GFP-S-Maleimide- | Lipofectamine | 200 ng eGFP- | 4.76 μL, 50 mM |
| PEG19-Maleimide- | 3000 | S-Maleimide- | Sodium Citrate, |
| mCherry mRNA | | PEG19- | pH 5.5 |
| bioconjugate | | Maleimide- | |
| | | mCherry | |

After transfection, the cells are dissociated according to the method described above for the eGFP-S-Maleimide-SulfoCy5, and analyzed by flow cytometry. A blue excitation laser with a 530/30 emission filter (green channel) is used to probe eGFP. A red excitation laser with a 675/25 filter (red channel) is used to probe mCherry and eGFP-S-Maleimide-SulfoCy5-PEG19-Maleimide-S-mCherry.

### Results:

Similar to the flow cytometry method described above for eGFP-S-Maleimide-SulfoCy5 Å, a gate is used to removed data points arising from non-fluorescence cells. The plot is further sub-divided into quadrants. The horizontal bisecting line is placed at a fluorescence intensity (675/25 filter) of 300 (a.u.) at which <0.5% of recorded events reached a fluorescence intensity above this value. The vertical bisecting line is placed at a fluorescence intensity of 2000 (a.u.) at which <0.5% of recorded events reached a fluorescence intensity above this value. These are the thresholds above which events are considered red (675/25) or green (530/30) fluorescence, respectively. As such, all events are considered as one of four outcomes depending on the quadrant they appear: (i) upper left (UL)—positive red (675/25) fluorescence; (ii) upper right (UR)—positive red (675/25) fluorescence and positive green (530/30) fluorescence; (iii) lower left (LL)—no fluorescence; and (iv) lower right (LR)—positive green (530/30) fluorescence.

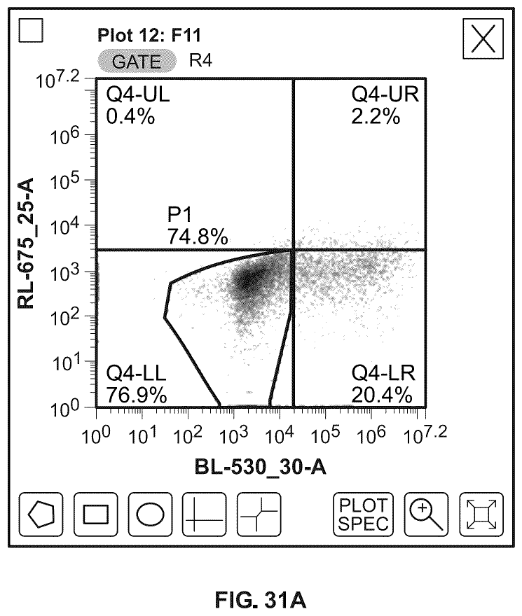

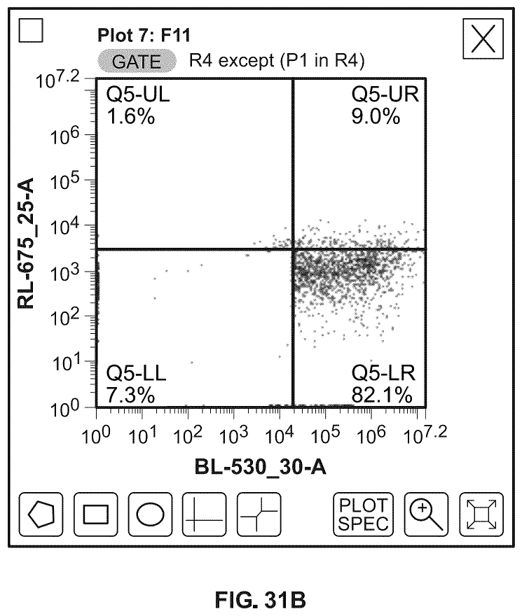

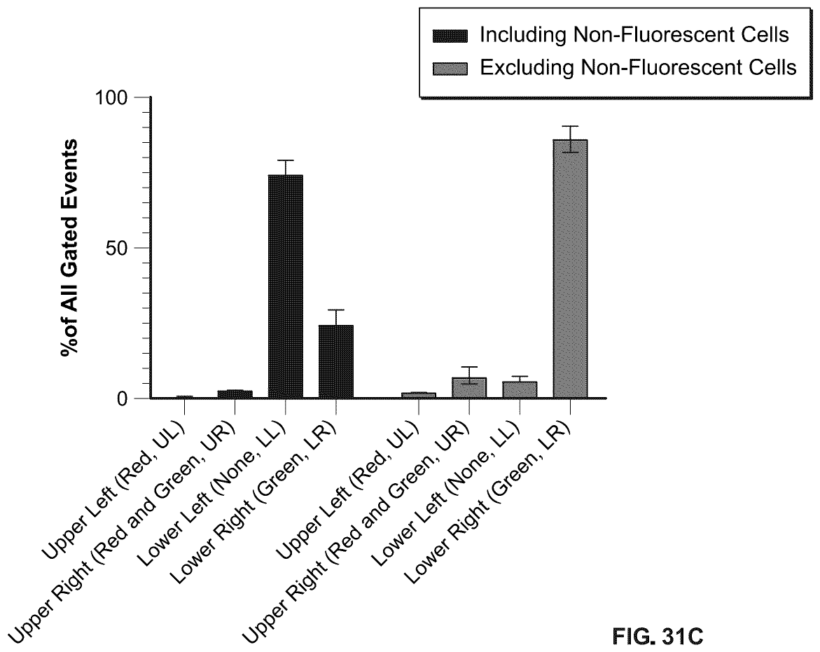

A majority of cells (74%) show no significant fluorescence in either channel. As shown in FIGS. 31A-C, for cells transfected with eGFP mRNA, the majority of cells that fluoresce (86% average) produce fluorescence in the green channel only (FIG. 31B lower right quadrant). FIG. 31C shows a bar graph that provides the average % of all gated events within a given quadrant for the triplicate runs along with a SD.

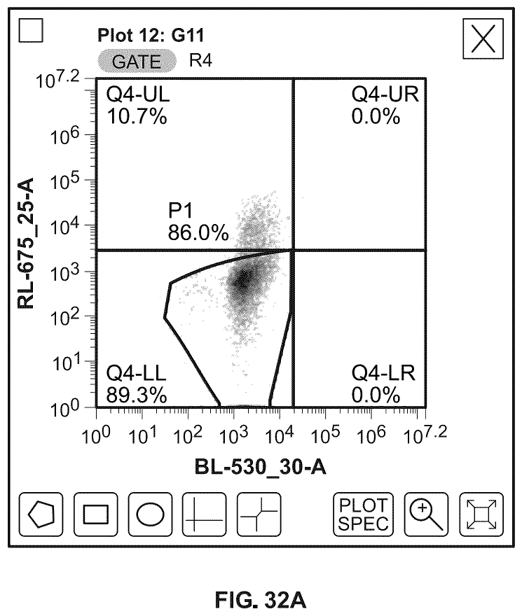

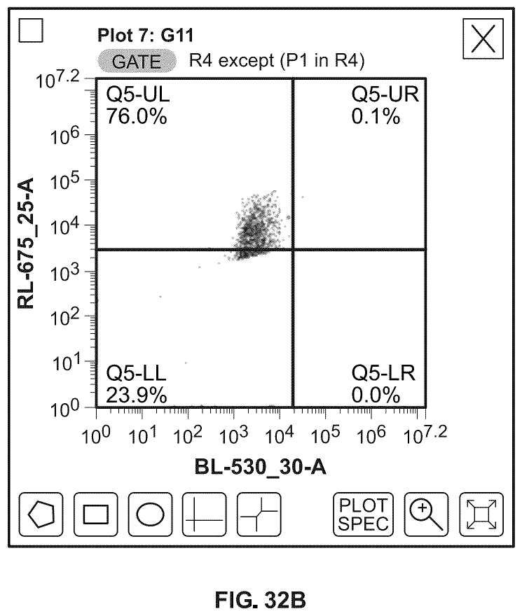

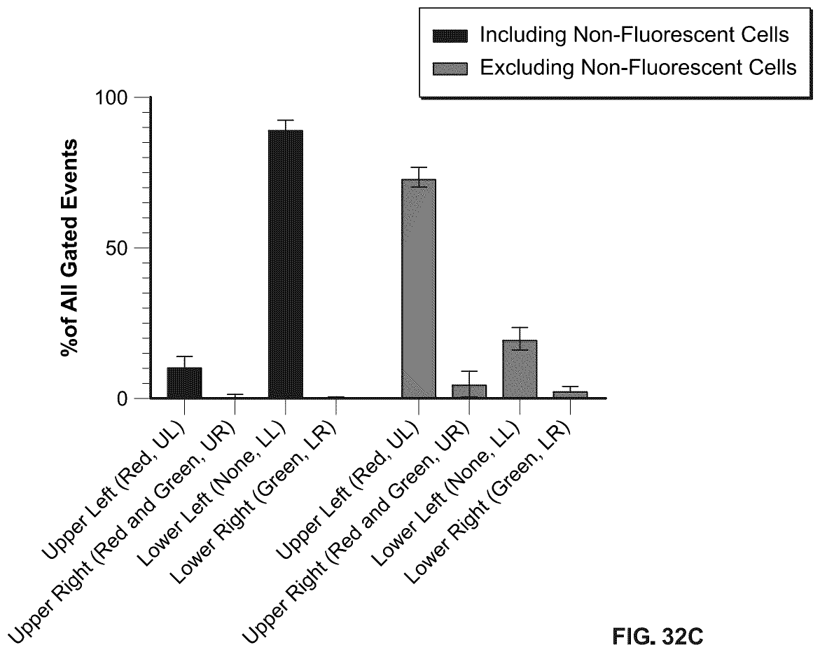

As shown in FIGS. 32A-C, for cells transfected with mCherry mRNA, the majority of cells that fluoresce (73% average) produce fluorescence in the red channel only (FIG. 32B upper left quadrant) while most events remain non-fluorescent (88% average).

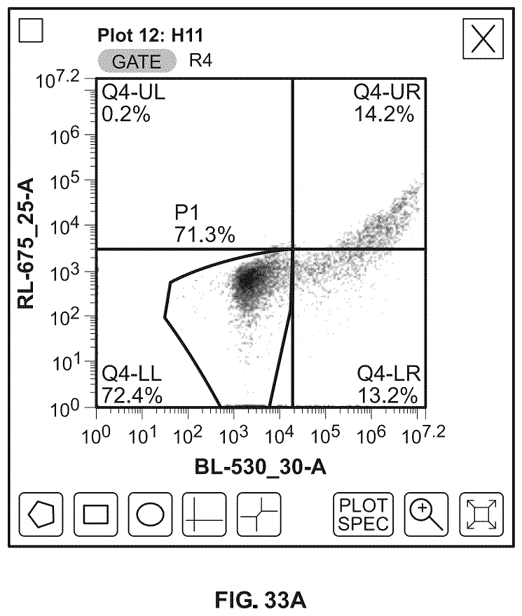

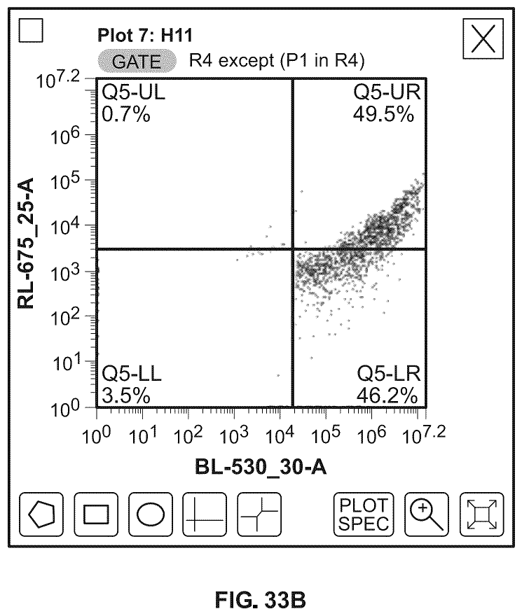

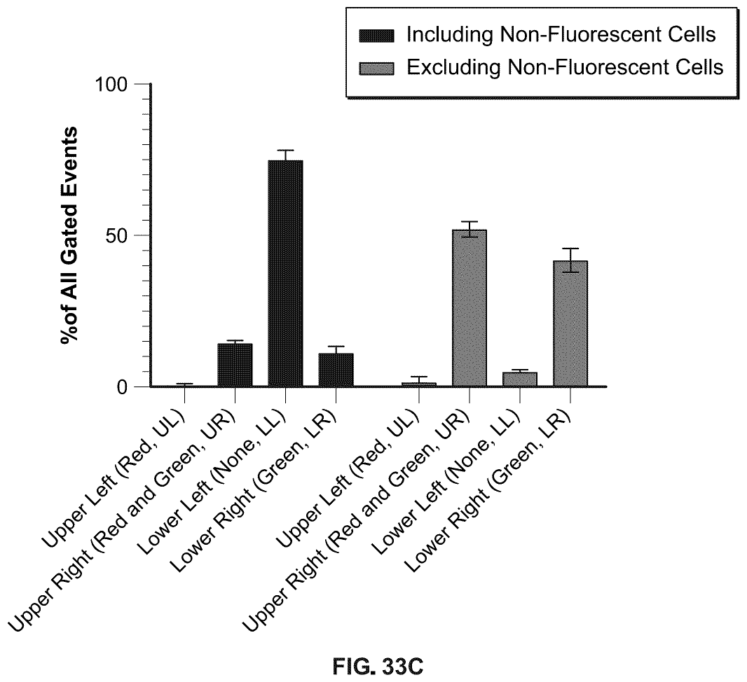

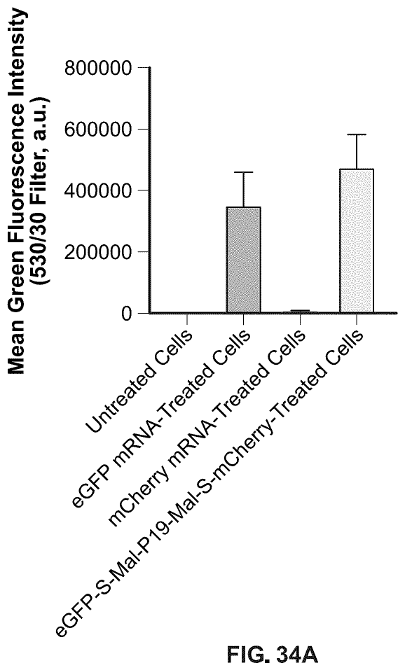

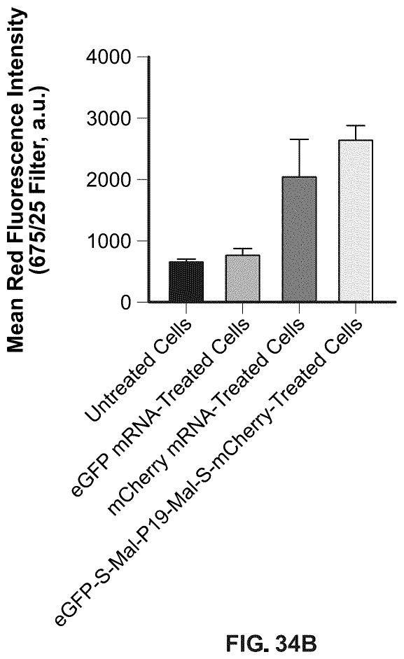

As shown in FIG. 33A-C, for cells transfected with eGFP-S-Maleimide-PEG19-Maleimide-S-mCherry mRNA bioconjugate, most cells (75% average) do not fluoresce in either channel. However, for the cells that do fluoresce, 52% fluoresce in both the green and the red channels (FIG. 33B upper right quadrant). Thus, cells treated with eGFP-S-Maleimide-PEG19-Maleimide-S-mCherry mRNA are able to produce both eGFP and mCherry protein from the mRNA bioconjugate, comparable to cells treated with either mCherry or eGFP alone (FIGS. 33C, 34A-B).

Experiment: Flow Cytometry on Cells Transfected with eGFP-S-Maleimide-PEG19-Maleimide-S-mCherry using Lipofectamine MessengerMAX

HEK-293T cells are cultured in conditions identical to the eGFP-S-Maleimide-SulfoCy5 experiment above. Cells are transfected with mCherry and eGFP-S-Maleimide-PEG19-Maleimide-S-mCherry mRNA, or buffer (control), using Lipofectamine 3000 (Thermo Fisher Scientific) reagent according to the manufacturer's protocol, similar to the transfection protocol detailed above for the eGFP-S-Maleimide-SulfoCy5 experiment. For the control group, cells are treated with buffer only (9.09 μL, 50 mM Sodium Citrate, pH 5.5). For the treatment groups, cells are treated with 100 ng of mCherry mRNA (4.54 μL, 22.0 ng/μL in 50 mM Sodium Citrate, pH 5.5), or 200 ng of eGFP-S-Maleimide-PEG19-Maleimide-S-mCherry mRNA (9.09 μL, 22.0 ng/μL in 50 mM Sodium Citrate, pH 5.5). Since the bioconjugate eGFP-S-Maleimide-PEG19-Maleimide-mCherry is about twice the molecular weight of either eGFP or mCherry mRNA alone, cells are treated with twice the amount of bioconjugate to ensure approximately the same amount of each respective mRNA within the bioconjugate.

Cells are also transfected with eGFP mRNA, or a mixture of mCherry and eGFP using Lipofectamine MessengerMAX (Thermo Fisher Scientific) reagent according to the manufacturer's protocol. Volumes provided correspond with the amount of reagent and mRNA for a single well on a 96-well plate. Briefly, 0.30 μL of the Lipofectamine MessengerMAX reagent is diluted into 5.00 μL of minimal essential media (Opti-MEM, Thermo Fisher Scientific) in a first microcentrifuge tube and is incubated for 10 minutes. In a second microcentrifuge tube, the mRNA (5-400 ng at concentrations ranging 10-1000 ng/μL) is diluted into 5.00 μL of Opti-MEM. The contents of first microcentrifuge tube are added to the second microcentrifuge tube, mixed, and incubated at room temperature for 5 minutes. The mixture is then added directly to a well of plated cells. For eGFP, cells are treated with 100 ng of eGFP mRNA (4.54 μL, 22.0 ng/μL in 50 mM Sodium Citrate, pH 5.5). For the mCherry and eGFP mixture, cells are treated with 100 ng of eGFP mRNA (4.54 μL, 22.0 ng/μL in 50 mM Sodium Citrate, pH 5.5) and 100 ng of mCherry mRNA (4.54 μL, 22.0 ng/μL in 50 mM Sodium Citrate, pH 5.5). mRNA and derivatized mRNA are transfected using Lipofectamine MessengerMAX (Thermo Fisher Scientific) reagent according to the manufacturer's protocol.

TABLE 10
Transfection Reaction Conditions

| | Transfection | | |
|---|---|---|---|
| Treatment Group | Reagent | Payload | Buffer |
| Control | — | — | 9.09 μL, 50 mM |
| (Buffer only) | | | Sodium Citrate, |
| | | | pH 5.5 |
| eGFP mRNA | Lipofectamine | 100 ng EGFP | 4.54 μL, 50 mM |
| | Messen- | mRNA | Sodium Citrate, |
| | gerMAX | | pH 5.5 |
| mCherry mRNA | Lipofectamine | 100 ng | 4.54 μL, 50 mM |
| | 3000 | mCherry | Sodium Citrate, |
| | | mRNA | pH 5.5 |
| eGRP mRNA and | Lipofectamine | 100 ng eGFP | 9.09 μL, 50 mM |
| mCherry mRNA | Messen- | mRNA and 100 | Sodium Citrate, |
| | gerMAX | ng mCherry | pH 5.5 |
| | | mRNA | |
| eGFP-S-Maleimide- | Lipofectamine | 200 ng eGFP- | 9.09 μL, 50 mM |
| PEG19-Maleimide- | 3000 | S-Maleimide- | Sodium Citrate, |
| mCherry mRNA | | PEG19- | pH 5.5 |
| bioconjugate | | Maleimide- | |
| | | mCherry | |

After transfection, the cells are dissociated according to the method described above for the eGFP-S-Maleimide-SulfoCy5, and analyzed by flow cytometry. A blue excitation laser with a 530/30 emission filter (green channel) is used to probe eGFP. A red excitation laser with a 675/25 filter (red channel) is used to probe mCherry and eGFP-S-Maleimide-SulfoCy5-PEG19-Maleimide-S-mCherry.

### Results:

Similar to the flow cytometry method described above for eGFP-S-Maleimide-SulfoCy5 Å, a gate is used to removed data points arising from non-fluorescence cells. The plot is further sub-divided into quadrants. The horizontal bisecting line is placed at a fluorescence intensity (675/25 filter) of 300 (a.u.) at which <0.5% of recorded events reached a fluorescence intensity above this value. The vertical bisecting line is placed at a fluorescence intensity of 2000 (a.u.) at which <0.5% of recorded events reached a fluorescence intensity above this value. These are the thresholds above which events are considered red (675/25) or green (530/30) fluorescence, respectively. As such, all events are considered as one of four outcomes depending on the quadrant they appear: (i) upper left (UL)—positive red (675/25) fluorescence; (ii) upper right (UR)—positive red (675/25) fluorescence and positive green (530/30) fluorescence; (iii) lower left (LL)—no fluorescence; and (iv) lower right (LR)—positive green (530/30) fluorescence.

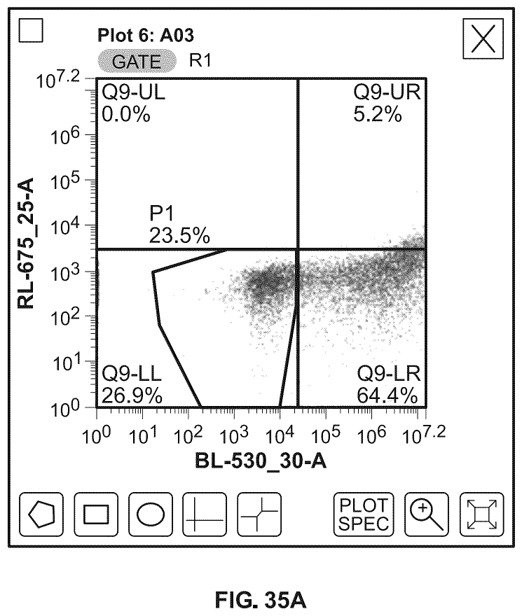

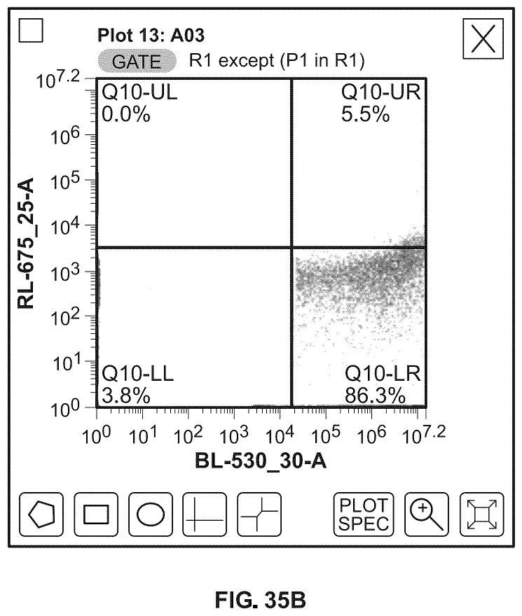

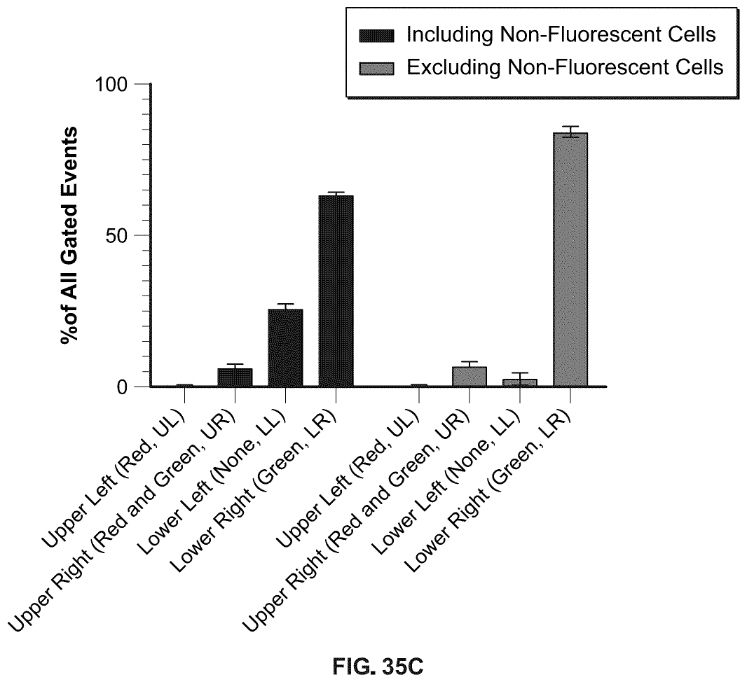

A majority of cells (97%) show no significant fluorescence in either channel. As shown in FIGS. 35A-C, for cells transfected with eGFP mRNA, the majority of cells that fluoresce (84% average if non-fluorescent cells are not included, or 63% average if non-fluorescent cells are included) produce fluorescence in the green channel only (FIG. 35B lower right quadrant). FIG. 35C provides the average % of all gated events within a given quadrant for the triplicate runs along with a SD.

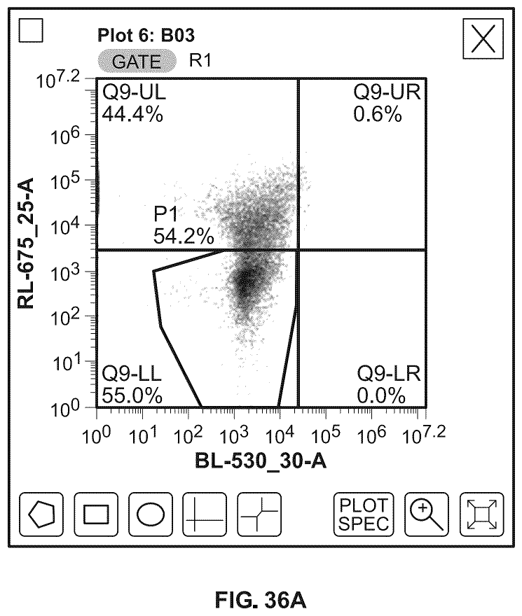

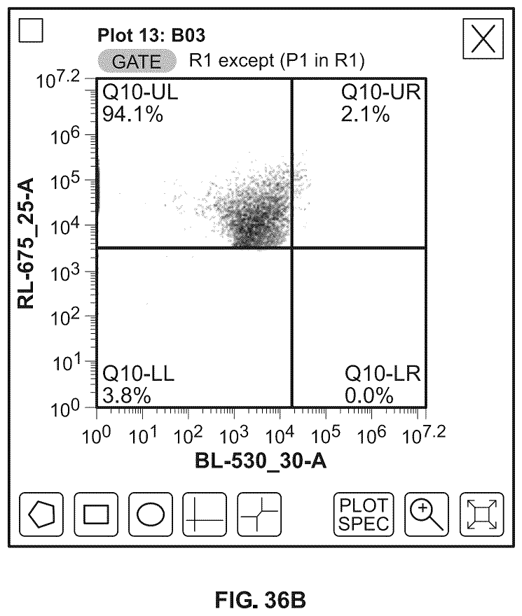

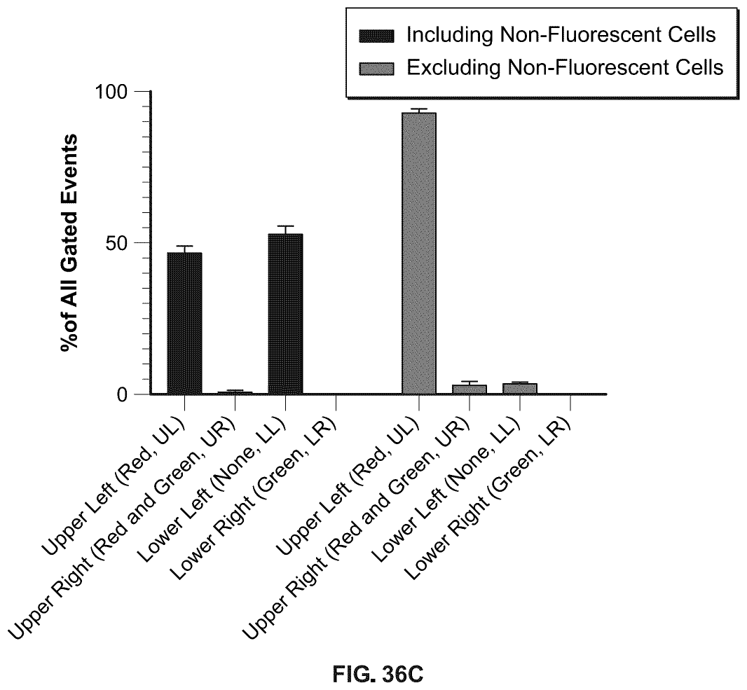

As shown in FIGS. 36A-C, for cells transfected with mCherry mRNA, the majority of cells that fluoresce (93% average) produce fluorescence in the red channel only (FIG. 36B upper left quadrant), while a majority of events are non-fluorescent (53% average). FIG. 36C provides the average % of all gated events within a given quadrant for the triplicate runs along with a SD.

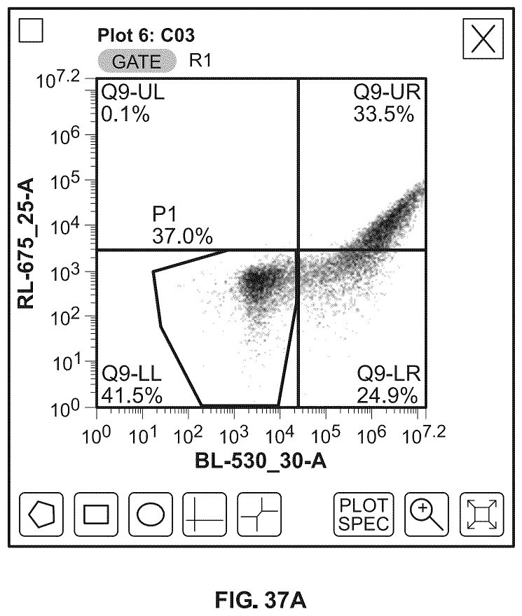

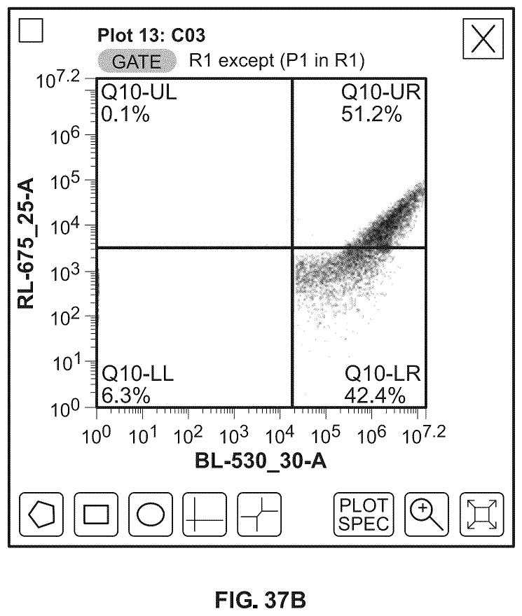

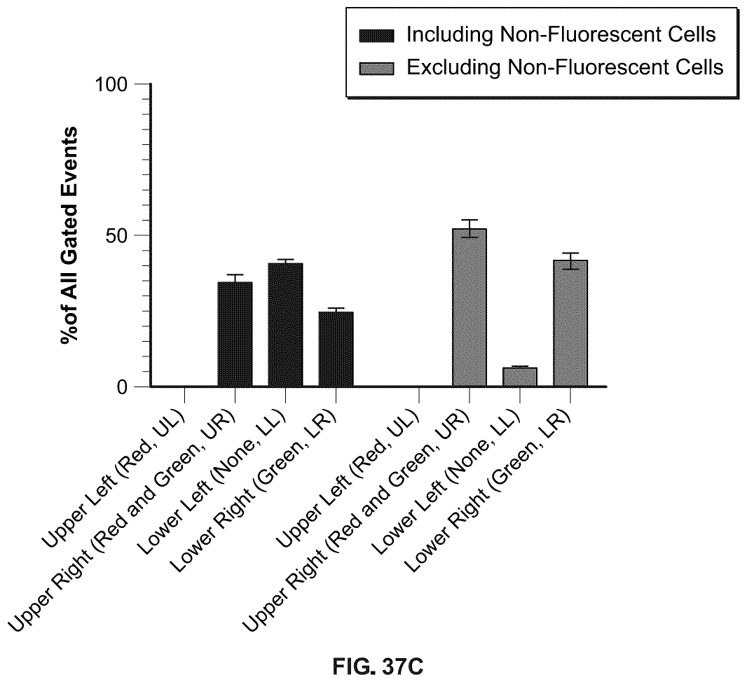

As shown in FIGS. 37A-C, for cells transfected with both eGFP mRNA and mCherry mRNA, the majority of cells that fluoresce (52% average) produce fluorescence in both red and green channels (FIG. 37B upper right quadrant), suggesting successful production of both eGFP and mCherry proteins from the two separate mRNAs. FIG. 37C provides the average % of all gated events within a given quadrant for the triplicate runs along with a SD.

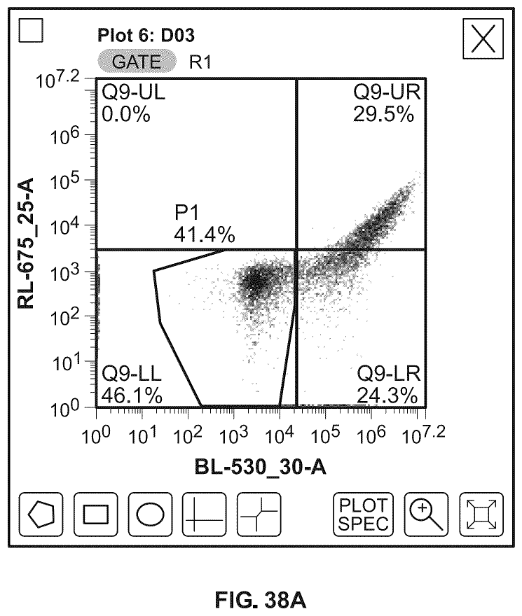

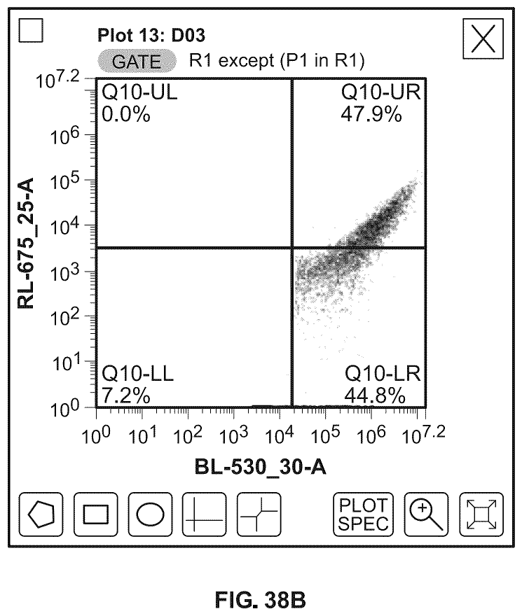

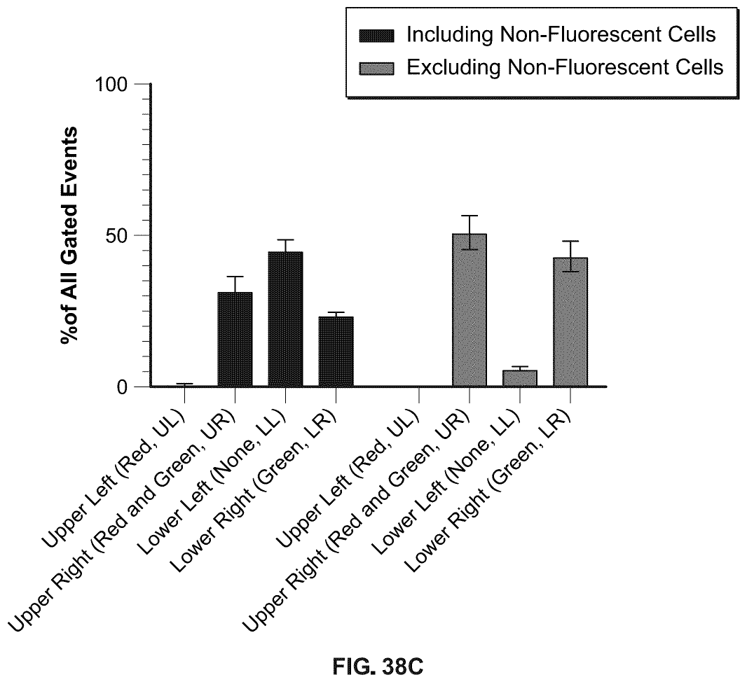

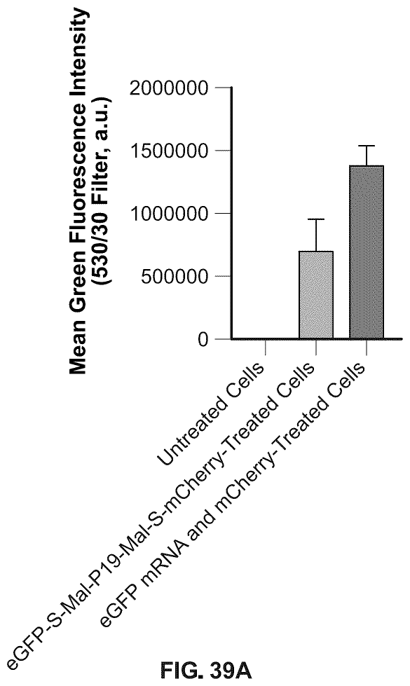

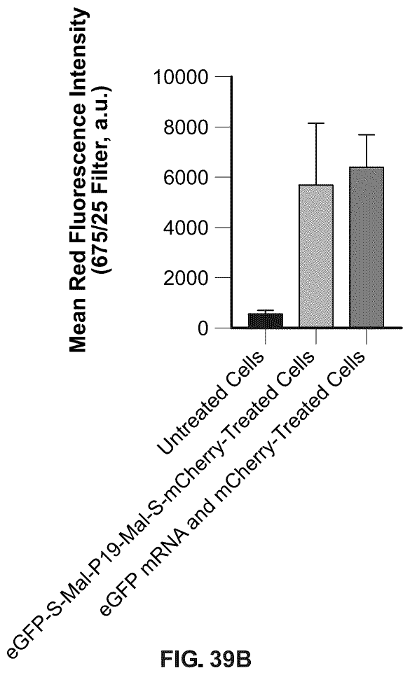

As shown in FIGS. 38A-C, most cells transfected with eGFP-S-Maleimide-PEG19-Maleimide-S-mCherry mRNA bioconjugate do not fluoresce in either channel (45% average), but for the cells that do fluoresce, 51% fluoresce in both the green and the red channels (FIG. 38B upper right quadrant), suggesting successful production of both eGFP and mCherry proteins from the mRNA bioconjugate (FIGS. 38C, 39A-B). FIG. 38C provides the average % of all gated events within a given quadrant for the triplicate runs along with a SD.

FIG. 39A-B show the gated flow cytometry data from all three samples in bar graph form. FIG. 39A and FIG. 39B provide the mean fluorescence intensity (a.u.) for the green (530/30 filter) and red (675/25 filter) channels, respectively, for the triplicate runs along with a SD. It appears that the cells treated with eGFP-S-Maleimide-PEG19-Maleimide-S-mCherry generate a significant fluorescence intensity in both the red and green channels comparable to the cells treated with both mCherry and eGFP, suggesting the conjugate is similarly resulting in the production of both fluorescent proteins.

Formulation of mRNA Lipid Nanoparticles

Formulation of mRNA into LNPS is achieved using an ElveFlow OB1 Mk4 microfluidic device. This device is used to combine an aqueous solution containing the mRNA (133 ng/μL, 100 mM sodium citrate, pH 3.0) with an ethanolic lipid solution (12.5 mM solution of D-Lin-MC3-DMA, DSPC, cholesterol, and DSPE-PEG2k at a molar ratio of 50:10:38.5:1.5) through a herringbone mixer at a flow ratio of 3:1 at a total flow rate of 3.6 mL/minute. Lipids are purchased from BroadPharm®, and cholesterol is purchased from Millipore Sigma.

After formulation, the mRNA LNP solution is diluted 40× v/v with DPBS and buffer exchanged by dialysis (Slide-A-Lyzer™ MINI Dialysis Devices, 20K MWCO, ThermoFisher) for 2 hours followed by a dialysis buffer exchange and another 2 hour incubation. Following this, the mRNA LNP solution is taken up in a syringe and passed through a 0.2 μm PES syringe filter (Millipore Sigma), collected, and stored at 4° C. until further use. For animal experiments, the same general procedure is used except with sterile material and phosphate buffered saline (PBS, Corning) instead of DPBS.

Alternatively, formulation of mRNA into LNPS is achieved using a PreciGenome Flex-S Nanoparticle Synthesis System. In all cases using the Flex-S system, the aqueous component contains the mRNA (133 ng/μL, 20 mM sodium citrate, pH 3.0) while the solvent component contains one of the two following lipid mixtures in ethanol (99%, Millipore Sigma):
Lipid Mixture 1: 20.0 mM solution of D-Lin-MC3-DMA, DSPC, cholesterol, and DSPE-PEG2k at a molar ratio of 50:10:38.5:1.5

Lipid Mixture 2: 16.0 mM solution of ALC-0315, DSPC, cholesterol, and DSPE-PEG2k at a molar ratio of 46.3:9.4:1.6:42.7

The aqueous and solvent components are then combined into a microfluidic mixer chip (CHP-MIX-4) using the Flex-S system at a flow ratio of 3:1 at a total flow rate of 3.0 mL/minute. D-Lin-MC3-DMA, DSPC, and DSPE-PEG2k lipids are purchased from BroadPharm®. ALC-0315 is purchased from MedChemExpress. Cholesterol is purchased from Millipore Sigma.

After formulation, the mRNA LNP solution is either directly subjected to buffer exchanged or first diluted 1:1 or 1:3 v/v with sterile PBS followed by exchange. In both cases, buffer exchange is carried out using the appropriately sized Slide-A-Lyzer™ MINI Dialysis Devices, 20K MWCO, (ThermoFisher) with respect to the volume of the formulated mRNA LNP solution. Dialysis containers are left to incubate in an Eppendorf ThermoMixer C (16° C., 300 rpm) for 3 hours total with a dialysis buffer exchange and after the first and second hour of incubation. Following this, the mRNA LNP solution is taken up in a syringe and passed through a 0.2 μm PES syringe filter (Millipore Sigma), collected in a sterile tube, and stored at 4° C. until subsequent use. At this point, a sample of the mRNA LNP solution is acquired for characterization.

Encapsulation efficiency of formulated mRNA LNPs is calculated via mRNA concentration in solution using a Quant-iT RiboGreen assay (ThermoFisher). Briefly, the assay is used to measure the concentration of mRNA in two samples: 1) the non-perturbated sample, which is the sample diluted in TE buffer (10 mM Tris-HCl, 1 mM EDTA, pH 8.0) where LNPs should remain intact; 2) the perturbated sample, which is the sample diluted in TE with 2% v/v Triton™ X-100 where LNPs should fail to maintain their nanoparticle structure, releasing any encapsulated mRNA. From this, the concentration of the non-encapsulated mRNA (1) and the total concentration of mRNA (2) can be measured from which the encapsulation efficiency can be calculated.

Further mRNA LNP characterization includes size and polydispersity which are measured using dynamic light scattering (DLS) on a Wyatt Mobius™ instrument. Briefly, mRNA LNP solution is diluted 1:9 v/v in PBS, transferred to a 45 μL quartz cuvette, and analyzed using the instrument (10 acquisitions with an acquisition time of 10 seconds each). The resultant data is then analyzed using Wyatt DYNAMICS v7.3 software.

The specific procedures for generating LNPs for each bioconjugate of interest is as follows:

### Specific Procedure for Generation of FLuc LNP 1

The general procedure for the formulation of mRNA LNPs with the Flex-S system is followed using Lipid Mixture 1. Briefly, FLuc is diluted to 133 ng/μL in 20 mM sodium citrate (pH 3.0) before mixing with Lipid Mixture 1 using the Flex-S system as described. This generates 200 μL of the formulated solution of LNPs which is diluted 1:3 (v/v) with 600 μL of sterile PBS before it is subjected to dialysis with a 2.0 mL Slide-A-Lyzer™ MINI Dialysis Device. After buffer exchange, filtration and collection (see the general procedure for details), the final solution of FLuc LNP 1 has the following characteristics: z-average, 108 nm; PDI, 0.02; encapsulation efficiency, 99%.

### Specific Procedure for Generation of FLuc LNP 2

The general procedure for the formulation of mRNA LNPs with the Flex-S system is followed using Lipid Mixture 2. Briefly, FLuc is diluted to 133 ng/μL in 20 mM sodium citrate (pH 3.0) before mixing with Lipid Mixture 2 using the Flex-S system as described. This generates 200 μL of the formulated solution of LNPs which is diluted 1:3 (v/v) with 600 μL of sterile PBS before it is subjected to dialysis with a 2.0 mL Slide-A-Lyzer™ MINI Dialysis Device. After buffer exchange, filtration and collection (see the general procedure for details), the final solution of FLuc LNP 2 has the following characteristics: z-average, 93 nm; PDI, 0.17; encapsulation efficiency, 97%.

### Specific Procedure for Generation of FLuc (Mock Ligation) LNP 1

The general procedure for the formulation of mRNA LNPs with the Flex-S system is followed using Lipid Mixture 1. Briefly, FLuc (Mock Ligation) is diluted to 133 ng/μL in 20 mM sodium citrate (pH 3.0) before mixing with Lipid Mixture 1 using the Flex-S system as described. This generates 200 μL of the formulated solution of LNPs which is diluted 1:3 (v/v) with 600 μL of sterile PBS before it is subjected to dialysis with a 2.0 mL Slide-A-Lyzer™ MINI Dialysis Device. After buffer exchange, filtration and collection (see the general procedure for details), the final solution of FLuc (Mock Ligation) LNP 1 has the following characteristics: z-average, 89 nm; PDI, 0.08; encapsulation efficiency, 98%.

### Specific Procedure for Generation of FLuc-SH LNP 1

The general procedure for the formulation of mRNA LNPs with the Flex-S system is followed using Lipid Mixture 1. Briefly, FLuc-SH is diluted to 133 ng/μL in 20 mM sodium citrate (pH 3.0) before mixing with Lipid Mixture 1 using the Flex-S system as described. This generates 200 μL of the formulated solution of LNPs which is diluted 1:3 (v/v) with 600 μL of sterile PBS before it is subjected to dialysis with a 2.0 mL Slide-A-Lyzer™ MINI Dialysis Device. After buffer exchange, filtration and collection (see the general procedure for details), the final solution of FLuc-SH LNP 1 has the following characteristics: z-average, 102 nm; PDI, 0.02.

### Specific Procedure for Generation of FLuc-S-Maleimide-SulfoCy5 LNP 1

The general procedure for the formulation of mRNA LNPs with the Flex-S system is followed using Lipid Mixture 1. Briefly, FLuc-S-Maleimide-SulfoCy5 is diluted to 133 ng/μL in 20 mM sodium citrate (pH 3.0) before mixing with Lipid Mixture 1 using the Flex-S system as described. This generates 200 μL of the formulated solution of LNPs which is diluted 1:3 (v/v) with 600 μL of sterile PBS before it is subjected to dialysis with a 2.0 mL Slide-A-Lyzer™ MINI Dialysis Device. After buffer exchange, filtration and collection (see the general procedure for details), the final solution of FLuc-S-Maleimide-SulfoCy5 LNP 1 has the following characteristics: z-average, 99 nm; PDI, 0.07; encapsulation efficiency, 98%.

### Specific Procedure for Generation of FLuc-S-Maleimide-GalNAc LNP 1

The general procedure for the formulation of mRNA LNPs with the Flex-S system is followed using Lipid Mixture 1. Briefly, FLuc-S-Maleimide-GalNAc (SEQ ID NO: 88) is diluted to 133 ng/μL in 20 mM sodium citrate (pH 3.0) before mixing with Lipid Mixture 1 using the Flex-S system as described. This generates 200 μL of the formulated solution of LNPs which is diluted 1:3 (v/v) with 600 μL of sterile PBS before it is subjected to dialysis with a 2.0 mL Slide-A-Lyzer™ MINI Dialysis Device. After buffer exchange, filtration and collection (see the general procedure for details), the final solution of FLuc-S-Maleimide-GalNAc LNP 1 has the following characteristics: z-average, 86 nm; PDI, 0.01; encapsulation efficiency, 92%.

### Specific Procedure for Generation of FLuc-NC-ddnt LNP 1

The general procedure for the formulation of mRNA LNPs with the Flex-S system is followed using Lipid Mixture 1. Briefly, FLuc-NC-ddnt (SEQ ID NO: 82) is diluted to 133 ng/μL in 20 mM sodium citrate (pH 3.0) before mixing with Lipid Mixture 1 using the Flex-S system as described. This generates 200 μL of the formulated solution of LNPs which is diluted 1:3 (v/v) with 600 μL of sterile PBS before it is subjected to dialysis with a 2.0 mL Slide-A-Lyzer™ MINI Dialysis Device. After buffer exchange, filtration and collection (see the general procedure for details), the final solution of FLuc-NC-ddnt LNP 1 has the following characteristics: z-average, 98 nm; PDI, 0.02; encapsulation efficiency.

### Specific Procedure for Generation of FLuc-NC-SR LNP 1

The general procedure for the formulation of mRNA LNPs with the Flex-S system is followed using Lipid Mixture 1. Briefly, FLuc-NC-SR (SEQ ID NO: 83) is diluted to 133 ng/μL in 20 mM sodium citrate (pH 3.0) before mixing with Lipid Mixture 1 using the Flex-S system as described. This generates 200 μL of the formulated solution of LNPs which is diluted 1:3 (v/v) with 600 μL of sterile PBS before it is subjected to dialysis with a 2.0 mL Slide-A-Lyzer™ MINI Dialysis Device. After buffer exchange, filtration and collection (see the general procedure for details), the final solution of FLuc-NC-SR LNP 1 has the following characteristics: z-average, 103 nm; PDI, 0.03; encapsulation efficiency.

### Specific Procedure for Generation of FLuc-NC-SH LNP 1

The general procedure for the formulation of mRNA LNPs with the Flex-S system is followed using Lipid Mixture 1. First, FLuc-NC-SH (in 10 mM sodium citrate buffer pH 6.5) is heated at 70° C. for two minutes then cooled to 4° C. for two minutes. Following this, the solution is further diluted to 133 ng/μL in 20 mM sodium citrate (pH 3.0) before mixing with Lipid Mixture 1 using the Flex-S system as described. This generates 200 μL of the formulated solution of LNPs which is diluted 1:3 (v/v) with 600 μL of sterile PBS before it is subjected to dialysis with a 2.0 mL Slide-A-Lyzer™ MINI Dialysis Device. After buffer exchange, filtration and collection (see the general procedure for details), the final solution of FLuc-NC-SH LNP 1 has the following characteristics: z-average, 82 nm; PDI, 0.04; encapsulation efficiency, 94%.

### Specific Procedure for Generation of FLuc-NC-S-Maleimide-GalNAc LNP 1

The general procedure for the formulation of mRNA LNPs with the Flex-S system is followed using Lipid Mixture 1. First, FLuc-NC-S-Maleimide-GalNAc (in 10 mM sodium citrate buffer pH 6.5) is heated at 70° C. for two minutes then cooled to 4° C. for two minutes. Following this, the solution is further diluted to 133 ng/μL in 20 mM sodium citrate (pH 3.0) before mixing with Lipid Mixture 1 using the Flex-S system as described. This generates 200 μL of the formulated solution of LNPs which is diluted 1:3 (v/v) with 600 μL of sterile PBS before it is subjected to dialysis with a 2.0 mL Slide-A-Lyzer™ MINI Dialysis Device. After buffer exchange, filtration and collection (see the general procedure for details), the final solution of FLuc-NC-S-Maleimide-GalNAc has the following characteristics: z-average, 104 nm; PDI, 0.08; encapsulation efficiency, 97%.

### Specific Procedure for Generation of FLuc-NC-S-Maleimide-GalNAc LNP 2.1

The general procedure for the formulation of mRNA LNPs with the Flex-S system is followed using Lipid Mixture 2. Briefly, FLuc-NC-S-Maleimide-GalNAc is diluted to 133 ng/μL in 20 mM sodium citrate (pH 3.0) before mixing with Lipid Mixture 2 using the Flex-S system as described. This generates 200 μL of the formulated solution of LNPs which is diluted 1:3 (v/v) with 600 μL of sterile PBS before it is subjected to dialysis with a 2.0 mL Slide-A-Lyzer™ MINI Dialysis Device. After buffer exchange, filtration and collection (see the general procedure for details), the final solution of FLuc-NC-S-Maleimide-GalNAc LNP 1 has the following characteristics: z-average, 98 nm; PDI, 0.1; encapsulation efficiency, 82%.

### Specific Procedure for Generation of FLuc-NC-S-Maleimide-GalNAc LNP 2.2

The general procedure for the formulation of mRNA LNPs with the Flex-S system is followed using Lipid Mixture 2. First, FLuc-NC-S-Maleimide-GalNAc (in 10 mM sodium citrate buffer pH 6.5) is heated at 70° C. for two minutes then cooled to 4° C. for two minutes. Following this, the solution is further diluted to 133 ng/μL in 20 mM sodium citrate (pH 3.0) before mixing with Lipid Mixture 2 using the Flex-S system as described. This generates 200 μL of the formulated solution of LNPs which is diluted 1:3 (v/v) with 600 μL of sterile PBS before being subjected to dialysis with a 2.0 mL Slide-A-Lyzer™ MINI Dialysis Device. After buffer exchange, filtration and collection (see the general procedure for details), the final solution of FLuc-NC-S-Maleimide-GalNAc LNP 1 has the following characteristics: z-average, 103 nm; PDI, 0.1; encapsulation efficiency, 78%.

### Example 3: In Vivo Biological Data for Oligonucleotide Bioconjugates

Formulation of mRNA bioconjugates in Lipid Nanoparticles (LNPs)

mRNA bioconjugates are formulated in lipid nanoparticles (LNPs) for delivery in cells and animals as described in Example 2 above. mRNA bioconjugates formulated in LNPs can be used directly on adherent cells by dispensing a volume of (200-1000 ng/μL) directly into a well of plated cells. The volume to be dispensed depends on the concentration of mRNA LNPs.

### General Study Design for Assessing Delivery and Expression Efficiency of FLuc-mRNA LNP in Mice

To test the delivery and expression efficiency of the oligonucleotide bioconjugates provided herein, animals are treated with a solution of mRNA LNPs encoding the protein firefly luciferase (FLuc) via intravenous (IV) tail injection. At a later, specific time point (or multiple timepoints), the animal is injected (intraperitoneally) with D-luciferin (150 mg/kg), anesthetized (isoflurane), and imaged within 10 minutes of D-luciferin injection with a PerkinElmer Xenogen IVIS Spectrum Imaging System. The general health, body weight, and mortality of the mice are also monitored. If the imaging event is the terminal event of the study, the animal is euthanized via CO2 asphyxiation and/or cervical dislocation. Following euthanasia, the following organs are collected and imaged ex vivo using the same IVIS Imaging system: liver, kidney, heart, spleen, pancreas, lung, and brain.

### IVIS Study Design 1

16 8-week old BALB/c mice are treated encapsulated mRNAs synthesized and formulated in LNPs by the methods described herein.

Briefly, 4 groups of 4 mice each (8 weeks old) are given a single IV dose of the test reagent on day 0. The test reagents are shown in Table 11.

TABLE 11
Test reagents

| | Animal | | | | |
|---|---|---|---|---|---|
| | | Num- | Treatment | | |
| Group | Type | ber | Test Reagent | Dosage | Regimen |
| 1 | BALB/ | 4 | PBS (Negative Control) | 100 μL | Single |
| 2 | c, male, | 4 | FLuc (Mock Ligation) in | 2.5 μg | IV dose |
| | 8 weeks | | LNP 1 (Positive Control) | mRNA/ | on day 0 |
| 3 | old | 4 | FLuc-S-Maleimide- | mouse | |
| | | | GalNAc in LNP 1 | | |
| 4 | | 4 | FLuc-S-Maleimide- | | |
| | | | SulfoCy5 in LNP 1 | | |

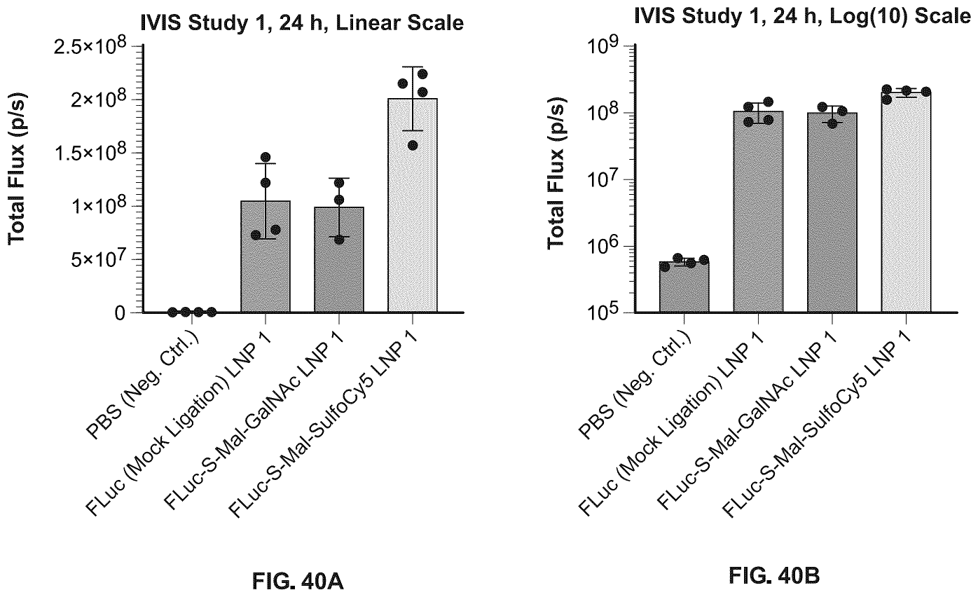

The results from imaging are shown in FIG. 40A and FIG. 40B.

### IVIS Study Design 2

15 8-week old BALB/c mice are treated encapsulated mRNAs synthesized and formulated in LNPs by the methods described herein.

Briefly, 5 groups of 3 mice each (8 weeks old) are given a single IV dose of the test reagent on day 0. The test reagents are shown in Table 12.

TABLE 12
Test reagents

| | Animal | | | | |
|---|---|---|---|---|---|
| | | Num- | Treatment | | |
| Group | Type | ber | Test Reagent | Dosage | Regimen |
| 1 | BALB/ | 3 | PBS (Negative Control) | 100 μL | Single |
| 2 | c, male, | 3 | FLuc (Mock Ligation) in | 2.5 μg | IV dose |
| | 8 weeks | | LNP 1 (Positive Control) | mRNA/ | on day 0 |
| 3 | old | 3 | FLuc-SH in LNP 1 | mouse | |
| 4 | | 3 | FLuc-NC-ddnt in LNP 1 | | |
| 5 | | 3 | FLuc-NC-SR in LNP 1 | | |

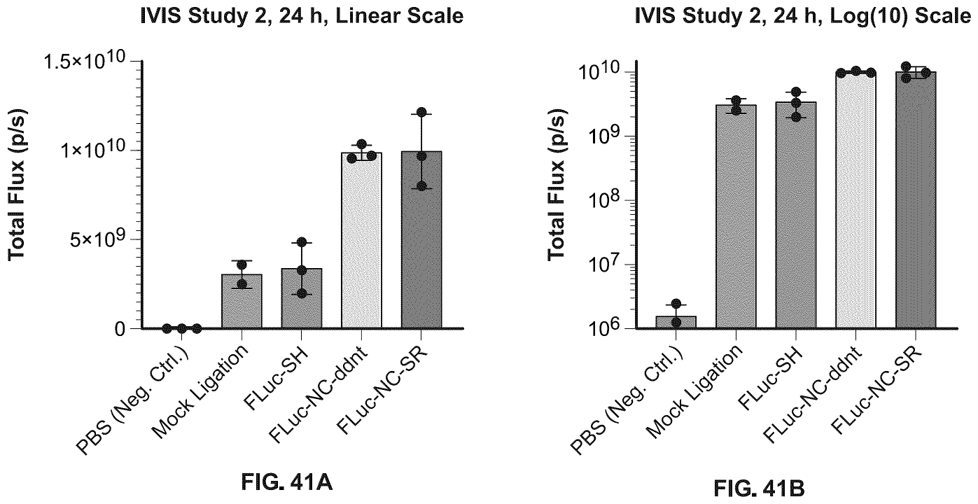

The results from imaging are shown in FIG. 41A and FIG. 41B.

### IVIS Study Design 3

12 8-week old BALB/c mice are treated encapsulated mRNAs synthesized and formulated in LNPs by the methods described herein.

Briefly, 4 groups of 3 mice each (8 weeks old) are given a single IV dose of the test reagent on day 0. The test reagents are shown in Table 13.

TABLE 13
Test reagents

| | Animal | | | | |
|---|---|---|---|---|---|
| | | Num- | Treatment | | |
| Group | Type | ber | Test Reagent | Dosage | Regimen |
| 1 | BALB/ | 3 | PBS (Negative Control) | 100 μL | Single |
| 2 | c, male, | 3 | FLuc in LNP 2 | 2.5 μg | IV dose |
| | 8 weeks | | (Alternative | mRNA/ | on day 0 |
| | | | Positive Control) | mouse | |
| 3 | | 3 | FLuc-NC-S-Maleimide- | | |
| | | | GalNAc in LNP 2.1 | | |
| 4 | | 3 | FLuc-NC-S-Maleimide- | | |
| | | | GalNAc in LNP 2.2 | | |

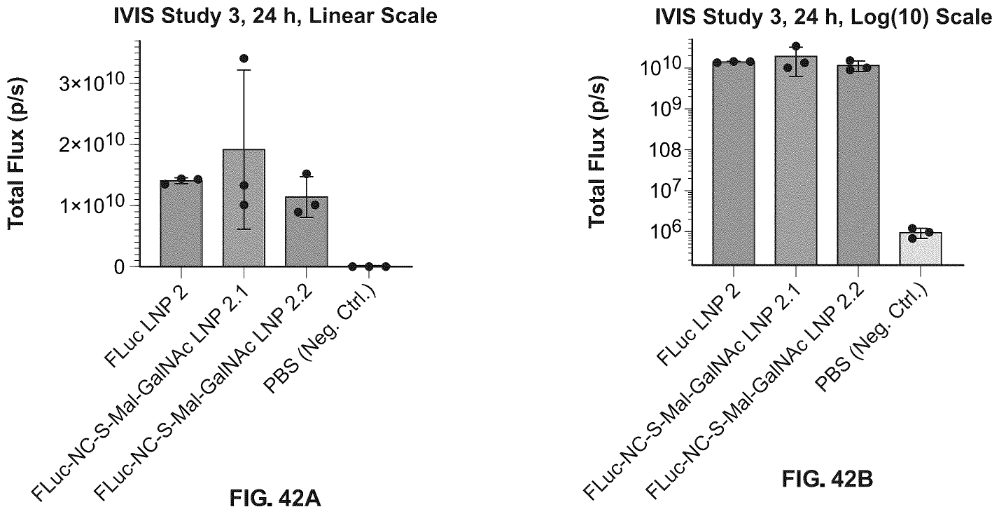

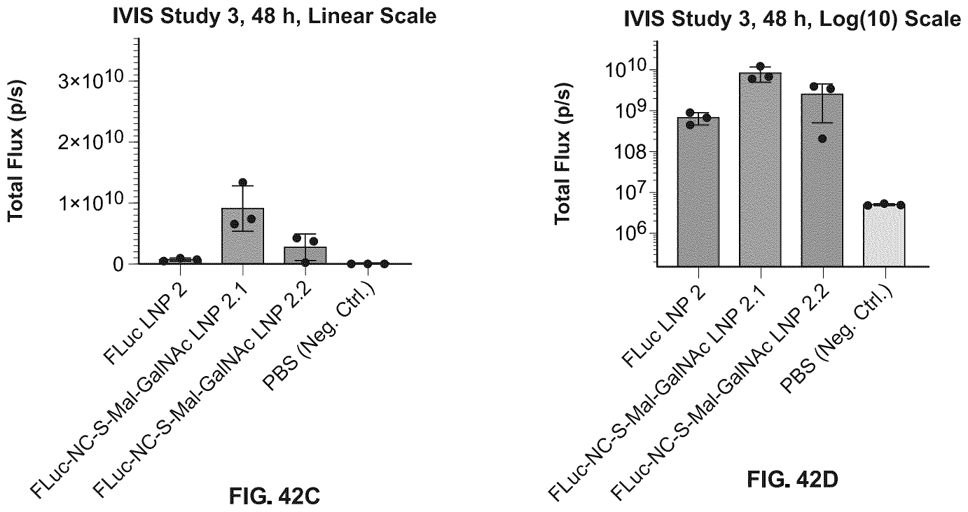

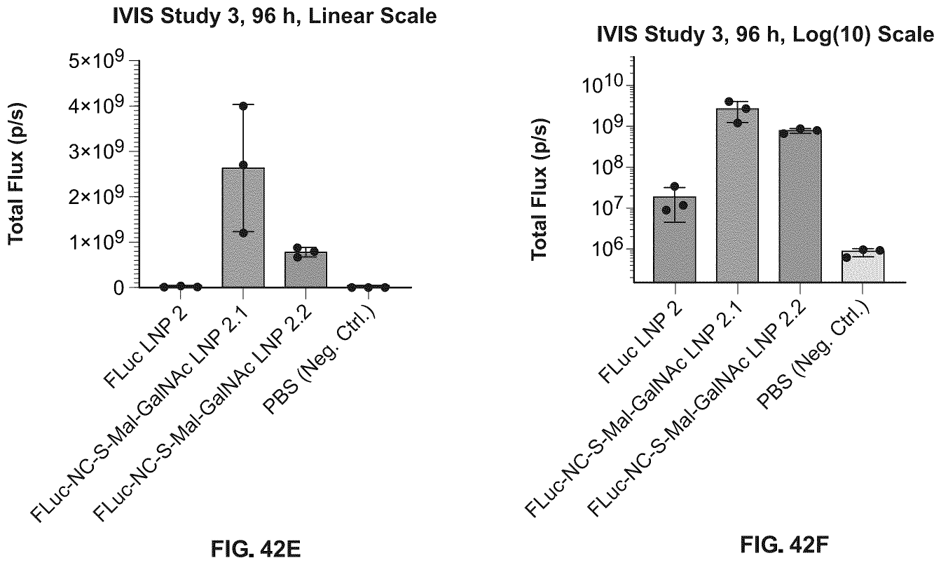

After 24, 48, and 96 hours, mice were treated with D-luciferin, anesthetized, and imaged. Following the whole-body in vivo imaging at 96 hours, mice were euthanized and organs were harvested for ex-vivo imaging. The results from imaging at 24 hours are shown in FIG. 42A and FIG. 42B. The results from imaging at 48 hours are shown in FIG. 42C and FIG. 42D. The results from imaging at 96 hours are shown in FIG. 42E and FIG. 42F.

### Example 4: Oligonucleotide Bioconjugates with Pembrolizumab

### PembrolizumabHC-SH

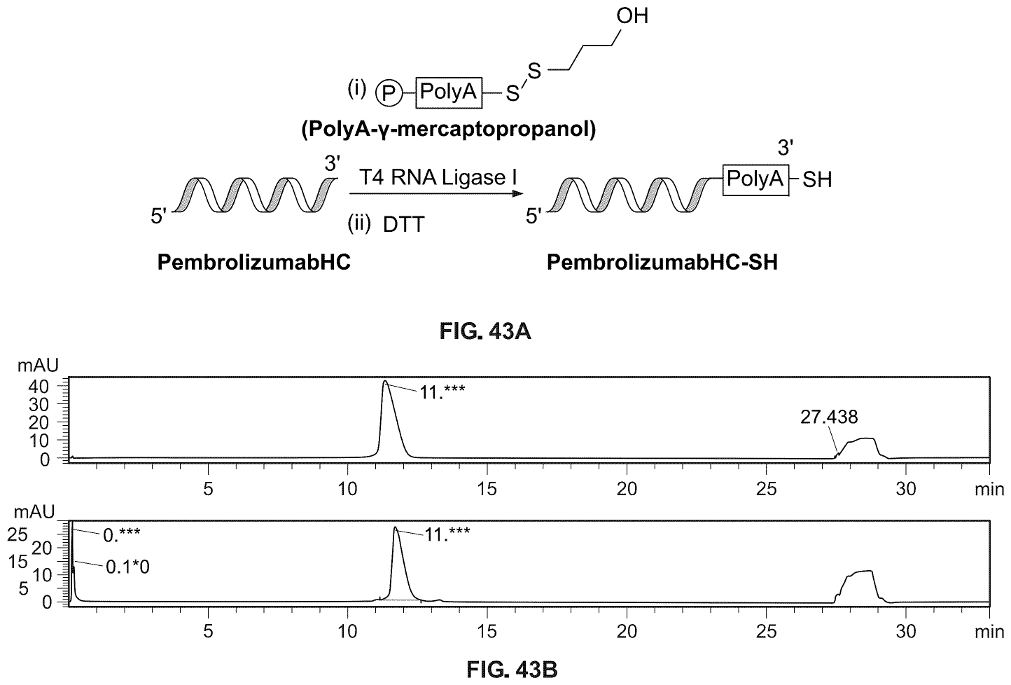

Bioconjugates comprising pembrolizumab heavy chain are synthesized using the general procedure for eGFP-SH described in Example 1, substituting eGFP-SH with PembrolizumabHC (FIG. 43A). Briefly, PembrolizumabHC (SEQ ID NO: 128; 150 μL, 1.6 μM solution in 10 mM Citric Acid Buffer, pH 6.5) is ligated to PolyA-γ-mercaptopropanol using T4 RNA Ligase I. Purification Method 3 is used to isolate the material (200 μL, 1.4 PM, 100% recovery). FIG. 43B shows the RP-HPLC chromatogram at an absorbance wavelength of 260 nm of PembrolizumabHC (top) and PembrolizumabHC-SH (bottom), analyzed using the parameters of Table 7.

### PembrolizumabLC-SH

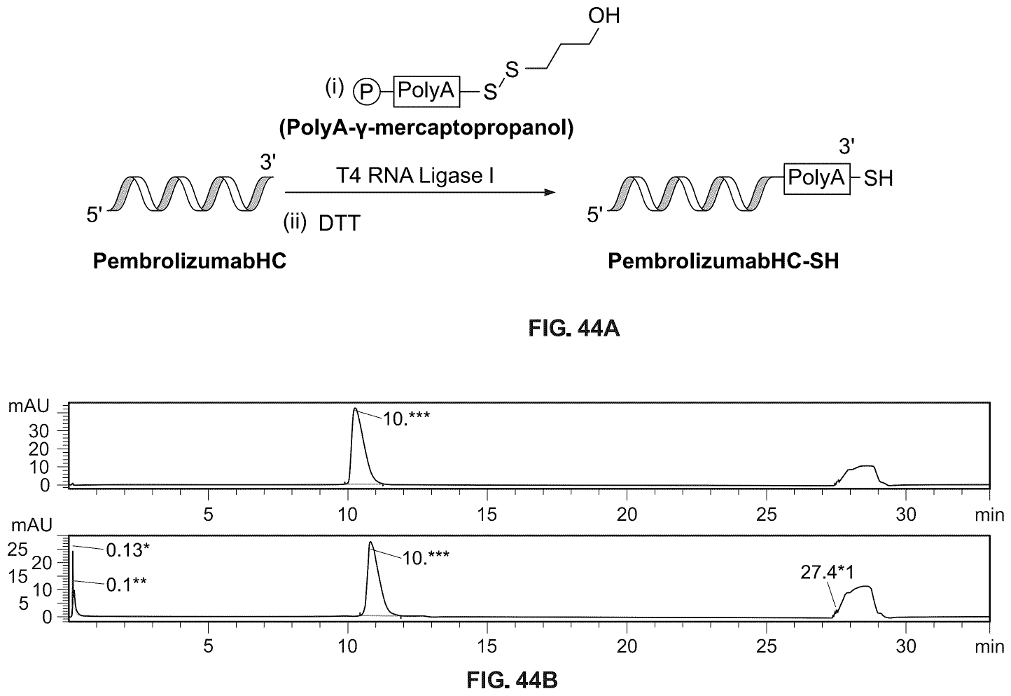

Bioconjugates comprising pembrolizumab light chain are synthesized using the general procedure for eGFP-SH described in Example 1, substituting eGFP-SH with PembrolizumabHC (FIG. 44A). Briefly, PembrolizumabLC (SEQ ID NO: 129; 150 μL, 1.6 μM solution in 10 mM Citric Acid Buffer, pH 6.5) is ligated to PolyA-γ-mercaptopropanol using T4 RNA Ligase I. Purification Method 3 is used to isolate the material (200 μL, 2.37 PM, 100% recovery). FIG. 44B shows the RP-HPLC chromatogram at an absorbance wavelength of 260 nm of PembrolizumabLC (top) and PembrolizumabLC-SH (bottom), analyzed using the parameters of Table 7.

### PembrolizumabHC-S-Maleimide-PEG19-Mal

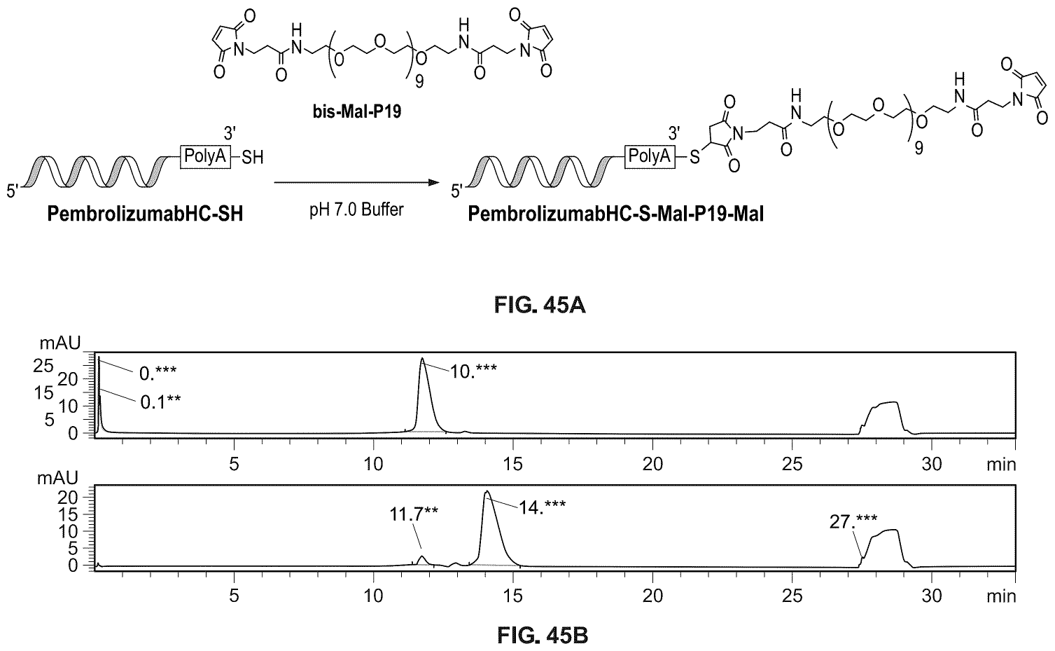

PembrolizumabHC-S-Maleimide-PEG19-Maleimide is synthesized as shown in FIG. 45A. Pembrolizumab-SH (110 μL, 772 ng/μL in Tris 1× pH 7.0 buffer, 85.5 μg, 155 μmol) is added to a 0.5 mL Eppendorf tube, followed by the addition of bis-Maleimide-PEG19 (2.1 μL, 10.0 mM in 9:1 water:DMSO ). The mixture is pipetted up and down 20 times. The mixture is allowed to incubate at room temperature for 1.5 h after which it was purified by Purification Method 3. The resulting solution of Pembrolizumab-S-Maleimide-PEG19-Maleimide (100 μL, 621 ng/μL in Tris 1× buffer, pH 7.0) can be used in subsequent studies/experiments without further purification or can be further purified using a centrifugal spin concentrator with a MWCO of ≤100 kDa. FIG. 45B shows the RP-HPLC chromatogram at an absorbance wavelength of 260 nm of Pembrolizumab-SH (top) and Pembrolizumab-S-Maleimide-PEG19-Maleimide (bottom), analyzed using the parameters of Table 7.

### PembrolizumabHC-S-Maleimide-PEG19-Maleimide-S-PembrolizumabLC

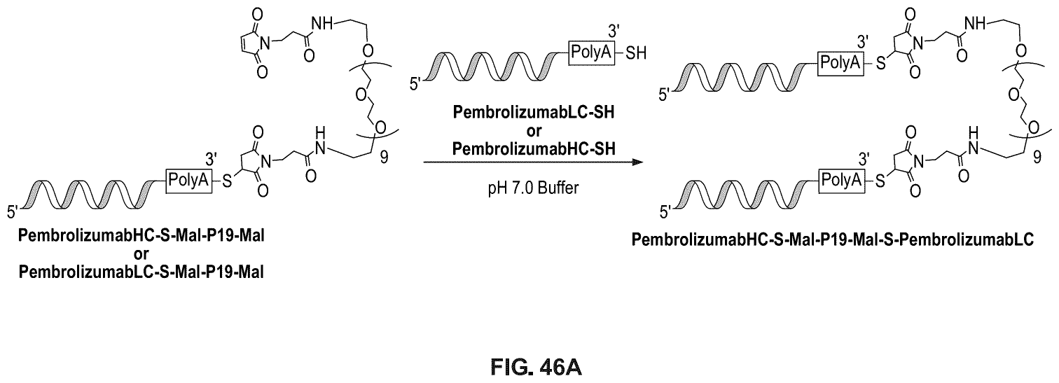

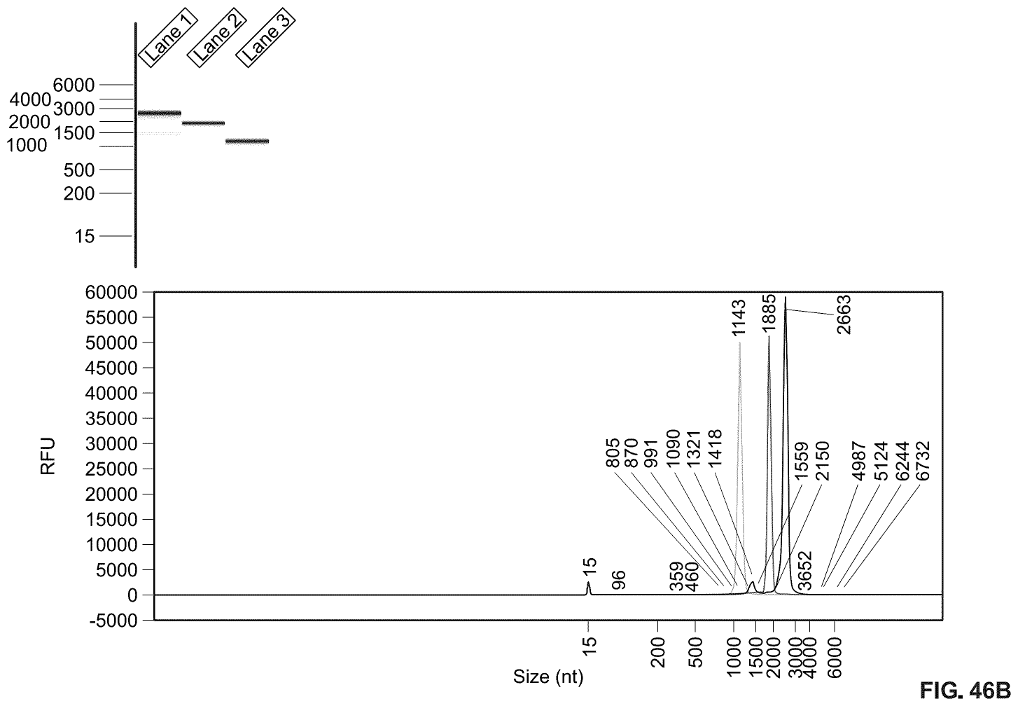

PembrolizumabHC-S-Maleimide-PEG19-Maleimide-S-PembrolizumabLC is synthesized as shown in FIG. 46A. Briefly, PembrolizumabHC-S-Maleimide-PEG19-Maleimide (110 μL, 721 ng/μL, 144 μmol) is added to a 0.5 mL Eppendorf tube, followed by PembrolizumabHC-SH (25 μL, 804 ng/μL, 63.6 μmol). The mixture is mixed by pipetting up and down 10 times. The mixture is incubated at room temperature for 0.5 h. After this time, the material is purified by SEC-HPLC and fractions containing PembrolizumabHC-S-Maleimide-PEG19-Maleimide-S-PembrolizumabLC are collected and concentrated. The collected fractions are subjected to analysis via capillary electrophoresis.

CE data is obtained from a Agilent Fragment Analyzer 5200 according to the general protocols. As shown in FIG. 46B (below), PembrolizumabHC-S-Maleimide-PEG19-Maleimide-S-PembrolizumabLC appears as a peak at 2663 nt, PembrolizumabHC shows up as a peak at 1885 nt, and PembrolizumabLC shows up as a peak at 1143 nt. The corresponding gel bands are also shown in FIG. 46B (top) where PembrolizumabHC-S-Maleimide-PEG19-Maleimide-S-PembrolizumabLC, PembrolizumabHC, and PembrolizumabLC are shown in Lanes 1, 2, and 3 respectively.

While the present disclosure has been described with reference to particular embodiments, it will be understood by those skilled in the art that various changes may be made and equivalents may be substituted for elements thereof without departing from the scope of the present disclosure. In addition, many modifications may be made to adapt a particular situation or material to the teachings of the present disclosure without departing from the scope of the present disclosure.

Therefore, it is intended that the present disclosure not be limited to the particular embodiments disclosed as the best mode contemplated for carrying out the present disclosure, but that the present disclosure will include all embodiments falling within the scope and spirit of the appended claims.

I. Sequences
SEQ ID NO: 1
(RKKRRQRRR)
SEQ ID NO: 2
(RQIKIWFQNRRMKWKK)
SEQ ID NO: 3
(RRRRRRRR)
SEQ ID NO: 4
(cyclo(FΦRRRRQ))
SEQ ID NO: 5
(KETWWETWWTEWSQPKKRK)
SEQ ID NO: 6
(GLAFLGFLGAAGSTMGAWSQPKKKRK)
SEQ ID NO: 7
(MVRRFLVTLRIRRACGPPRVR)
SEQ ID NO: 8
(MVKSKIGSWILVLFVAMWSDVGLCKKRPKP)
SEQ ID NO: 9
(KLALKALKALKAALKLA)
SEQ ID NO: 10
(GWTLNSAGYLLGKINLKALAALAKKIL)
SEQ ID NO: 11
(AGYLLGKINLKALAALAKKIL)
SEQ ID NO: 12
(GLWRALWRLLRSLWRLLWRA)
SEQ ID NO: 13
(KWLLRWLSRLLRWLARWLG)
SEQ ID NO: 14
(GLFEAIEGFIENGWEGMIDGWYGGGGRRRRRRRRRK)
SEQ ID NO: 15
(LSTAADMQGVVTDGMASGLDKDYLKPD)
SEQ ID NO: 16
(RRIRPRPPRLPRPRPRPLPFP)
SEQ ID NO: 17
(GIGAVLKVLTTGLPALISWIKRKRQQ)
SEQ ID NO: 18
(CSIPPEVKFNKPFVYLI)
SEQ ID NO: 19
(SDLWEMMMVSLACQ)
SEQ ID NO: 20
(LGAQSNF)
SEQ ID NO: 21
(PLILLRLLRGQF)
SEQ ID NO: 22
(PFVYLI)
SEQ ID NO: 23
(WSYGLRPG)
SEQ ID NO: 24
(WEARLARALARALARHLARALARALRACEA)
SEQ ID NO: 25
(Stearyl-AGYLLGKLLOOLAAAALOOLL)
SEQ ID NO: 26
(WEAKLAKALAKALAKHLAKALAKALKA)
SEQ ID NO: 27
(WEAALAEALAEALAEHLAEALAEALEALAA)
SEQ ID NO: 28
(IGKEFKRIVERIKRFLRELVRPLR)
SEQ ID NO: 29
(KKALLAHALHLLALLALHLAHALKKA)
SEQ ID NO: 30
(KKALLALALHHLAHLALHLALALKKA)
SEQ ID NO: 31
(KLLLLKLLLLKLLLLKLLLLK)
SEQ ID NO: 32
(RRXRRXRRXRRXRRX, where X represents Aib)
SEQ ID NO: 33
(RRRRRRRRR)
SEQ ID NO: 34
(MAPFASLASGILLLLSLITSSKA)
SEQ ID NO: 35
(MLLGPGHTLSAPALALAVTLTLLVRSASP)
SEQ ID NO: 36
(MLLSVPLLLGLLGLAAA)
SEQ ID NO: 37
(MQELRGILLCLLLAAAVPTTP)
SEQ ID NO: 38
(MRYVASYLLAALGGNS)
SEQ ID NO: 39
(MGKSPEAWCIVLFSVLASFSA)
SEQ ID NO: 40
(MASSGSVQQPRLVLLMLVLAGAARA)
SEQ ID NO: 41
(MRWKIIQLQYCFLLVPCMLTALEA)
SEQ ID NO: 42
(MLSRSLLCLALAWVARVGA)
SEQ ID NO: 43
(MRFSCLALLPGVALLLASARLAAA)
SEQ ID NO: 44
(MRVLWVLGLCCVLLTFGFVRA)
SEQ ID NO: 45
(MKFPMVAAALLLLCAVRA)
SEQ ID NO: 46
(MRSLLLASFCLLAVALA)
SEQ ID NO: 47
(MKILLLCVGLLLTWDNGMVLG)
SEQ ID NO: 48
(MLRISGRNMKVLFAAALIVGSVVFLLLPGPSVA)
SEQ ID NO: 49
(MAATVRRQRPRRLLCWTLVAVLLADLLALS)
SEQ ID NO: 50
(MKMGVRLAARAWPLCGLLLAALGGVCA)
SEQ ID NO: 51
(MWWRLWWLLLLLLLLWLALAAAA)
SEQ ID NO: 52
(MGWSLILLFLVAVATRVLS)
SEQ ID NO: 53
(MDFQVQIISFLLISASVIMSRG)
SEQ ID NO: 54
(MEFGLSWVFLVALFRGVQC)
SEQ ID NO: 55
(MKWVTFISLLFLFSSAYS)
SEQ ID NO: 56
(MKLPVRLLVLMFWIPAASA)
SEQ ID NO: 57
(MNLLLILTFVAAAVA)
SEQ ID NO: 58
(MGSAALLLWVLLLWVPSSRA)
SEQ ID NO: 59
(MTRLTVLALLAGLLASSRA)
SEQ ID NO: 60
(MWWRLWWLLLLLLLLWPMVWAAA)
SEQ ID NO: 61
(MKLPVRLLVLMFWIPASSS)
SEQ ID NO: 62
(MDMRVPAQLLGLLLLWLSGARC)
SEQ ID NO: 63
(MKYLLPTAAAGLLLLAAQPAMA)
SEQ ID NO: 64
(MGVKVLFALICIAVAEA)
SEQ ID NO: 65
(MPLLLLLPLLWAGALA)
SEQ ID NO: 66
(MRARALLAVLLLLLLVGIAAAA)
SEQ ID NO: 67
(MATATLLAVLLLLLLVGSAGGA)
SEQ ID NO: 68
(MRARALLVVLVLVVLLGVASSA)
SEQ ID NO: 69
(MPGPGAALLLLLLVLLGLGSAA)
SEQ ID NO: 70
(MTTTTVLLLLVLVVLAGLTSGA)
SEQ ID NO: 71
(QFGDFDPSVEEEEDL)
SEQ ID NO: 72
AUGGUGAGCAAGGGCGAGGAGGACAACAUGGCCAUCAUCAAGGAGUUCAUGCGG
UUCAAGGUGCACAUGGAGGGCAGCGUGAACGGCCACGAGUUCGAGAUCGAGGGC
GAGGGCGAGGGCCGGCCCUACGAGGGCACCCAGACCGCCAAGCUGAAGGUGACCA
AGGGCGGCCCCCUGCCCUUCGCCUGGGACAUCCUGAGCCCCCAGUUCAUGUACGG
CAGCAAGGCCUACGUGAAGCACCCCGCCGACAUCCCCGACUACCUGAAGCUGAGC
UUCCCCGAGGGCUUCAAGUGGGAGCGGGUGAUGAACUUCGAGGACGGCGGCGUG
GUGACCGUGACCCAGGACAGCAGCCUGCAGGACGGCGAGUUCAUCUACAAGGUGA
AGCUGCGGGGCACCAACUUCCCCAGCGACGGCCCCGUGAUGCAGAAGAAGACCAU
GGGCUGGGAGGCCAGCAGCGAGCGGAUGUACCCCGAGGACGGCGCCCUGAAGGGC
GAGAUCAAGCAGCGGCUGAAGCUGAAGGACGGCGGCCACUACGACGCCGAGGUGA
AGACCACCUACAAGGCCAAGAAGCCCGUGCAGCUGCCCGGCGCCUACAACGUGAA
CAUCAAGCUGGACAUCACCAGCCACAACGAGGACUACACCAUCGUGGAGCAGUAC
GAGCGGGCCGAGGGCCGGCACAGCACCGGCGGCAUGGACGAGCUGUACAAGAGCG
GCAACUGA
SEQ ID NO: 73
AUGGUGAGCAAGGGCGAGGAGCUGUUCACCGGGGUGGUGCCCAUCCUGGUCGAG
CUGGACGGCGACGUAAACGGCCACAAGUUCAGCGUGUCCGGCGAGGGCGAGGGCG
AUGCCACCUACGGCAAGCUGACCCUGAAGUUCAUCUGCACCACCGGCAAGCUGCC
CGUGCCCUGGCCCACCCUCGUGACCACCCUGACCUACGGCGUGCAGUGCUUCAGC
CGCUACCCCGACCACAUGAAGCAGCACGACUUCUUCAAGUCCGCCAUGCCCGAAG
GCUACGUCCAGGAGCGCACCAUCUUCUUCAAGGACGACGGCAACUACAAGACCCG
CGCCGAGGUGAAGUUCGAGGGCGACACCCUGGUGAACCGCAUCGAGCUGAAGGGC
AUCGACUUCAAGGAGGACGGCAACAUCCUGGGGCACAAGCUGGAGUACAACUACA
ACAGCCACAACGUCUAUAUCAUGGCCGACAAGCAGAAGAACGGCAUCAAGGUGAA
CUUCAAGAUCCGCCACAACAUCGAGGACGGCAGCGUGCAGCUCGCCGACCACUAC
CAGCAGAACACCCCCAUCGGCGACGGCCCCGUGCUGCUGCCCGACAACCACUACC
UGAGCACCCAGUCCGCCCUGAGCAAAGACCCCAACGAGAAGCGCGAUCACAUGGU
CCUGCUGGAGUUCGUGACCGCCGCCGGGAUCACUCUCGGCAUGGACGAGCUGUAC
AAGUAA
SEQ ID NO: 74
AUGGAGGACGCCAAGAACAUCAAGAAGGGCCCCGCCCCCUUCUACCCCCUGGAGG
ACGGCACCGCCGGCGAGCAGCUGCACAAGGCCAUGAAGCGGUACGCCCUGGUGCC
CGGCACCAUCGCCUUCACCGACGCCCACAUCGAGGUGGACAUCACCUACGCCGAG
UACUUCGAGAUGAGCGUGCGGCUGGCCGAGGCCAUGAAGCGGUACGGCCUGAACA
CCAACCACCGGAUCGUGGUGUGCAGCGAGAACAGCCUGCAGUUCUUCAUGCCCGU
GCUGGGCGCCCUGUUCAUCGGCGUGGCCGUGGCCCCCGCCAACGACAUCUACAAC
GAGCGGGAGCUGCUGAACAGCAUGGGCAUCAGCCAGCCCACCGUGGUGUUCGUGA
GCAAGAAGGGCCUGCAGAAGAUCCUGAACGUGCAGAAGAAGCUGCCCAUCAUCCA
GAAGAUCAUCAUCAUGGACAGCAAGACCGACUACCAGGGCUUCCAGAGCAUGUAC
ACCUUCGUGACCAGCCACCUGCCCCCCGGCUUCAACGAGUACGACUUCGUGCCCG
AGAGCUUCGACCGGGACAAGACCAUCGCCCUGAUCAUGAACAGCAGCGGCAGCAC
CGGCCUGCCCAAGGGCGUGGCCCUGCCCCACCGGACCGCCUGCGUGCGGUUCAGC
CACGCCCGGGACCCCAUCUUCGGCAACCAGAUCAUCCCCGACACCGCCAUCCUGA
GCGUGGUGCCCUUCCACCACGGCUUCGGCAUGUUCACCACCCUGGGCUACCUGAU
CUGCGGCUUCCGGGUGGUGCUGAUGUACCGGUUCGAGGAGGAGCUGUUCCUGCG
GAGCCUGCAGGACUACAAGAUCCAGAGCGCCCUGCUGGUGCCCACCCUGUUCAGC
UUCUUCGCCAAGAGCACCCUGAUCGACAAGUACGACCUGAGCAACCUGCACGAGA
UCGCCAGCGGCGGCGCCCCCCUGAGCAAGGAGGUGGGCGAGGCCGUGGCCAAGCG
GUUCCACCUGCCCGGCAUCCGGCAGGGCUACGGCCUGACCGAGACCACCAGCGCC
AUCCUGAUCACCCCCGAGGGCGACGACAAGCCCGGCGCCGUGGGCAAGGUGGUGC
CCUUCUUCGAGGCCAAGGUGGUGGACCUGGACACCGGCAAGACCCUGGGCGUGAA
CCAGCGGGGCGAGCUGUGCGUGCGGGGCCCCAUGAUCAUGAGCGGCUACGUGAAC
AACCCCGAGGCCACCAACGCCCUGAUCGACAAGGACGGCUGGCUGCACAGCGGCG
ACAUCGCCUACUGGGACGAGGACGAGCACUUCUUCAUCGUGGACCGGCUGAAGAG
CCUGAUCAAGUACAAGGGCUACCAGGUGGCCCCCGCCGAGCUGGAGAGCAUCCUG
CUGCAGCACCCCAACAUCUUCGACGCCGGCGUGGCCGGCCUGCCCGACGACGACG
CCGGCGAGCUGCCCGCCGCCGUGGUGGUGCUGGAGCACGGCAAGACCAUGACCGA
GAAGGAGAUCGUGGACUACGUGGCCAGCCAGGUGACCACCGCCAAGAAGCUGCGG
GGCGGCGUGGUGUUCGUGGACGAGGUGCCCAAGGGCCUGACCGGCAAGCUGGACG
CCCGGAAGAUCCGGGAGAUCCUGAUCAAGGCCAAGAAGGGCGGCAAGAUCGCCGU
GUGA
SEQ ID NO: 75
PolyA-γ-mercaptopropanol
/5Phos/AAAAAAAAAAAAAAAAdA/3ThioMC3-D/
Where 3ThioMC3-D is

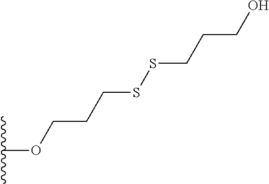 [SMILES: *OCCCSSCCCO]

attached via the 3′ end Hydroxyl. The
lowercase d before the nucleotide indicates a deoxynucleotide, e.g. dA is a deoxyadenosine.
SEQ ID NO: 76
PolyA-SH
/5Phos/AAAAAAAAAAAAAAAAdA/3ThioMC3Reduced-D/
Where 3ThioMC3Reduced-D is

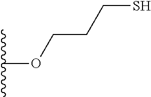 [SMILES: *OCCCS]

attached via the 3′ end Hydroxyl
SEQ ID NO: 77
Non-Canonical PolyA-γ-mercaptopropanol
/5Phos/AAAAAAAAAA/i2MOErA/*/i2MOErA/*/i2MOErA/*/i2MOErA/*/i2MOErA/*/i2MO
ErA/*/3ThioMC3-D/
Where /i2MOErA/* is

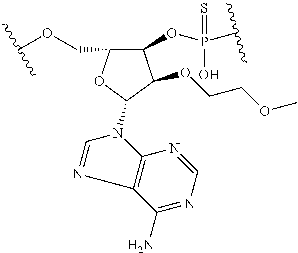 [SMILES: *OC[C@H]1O[C@@H](n2cnc3c(N)ncnc32)[C@H](OCCOC)[C@@H]1OP(*)(O)=S]

SEQ ID NO: 78
Non-Canonical PolyA-dideoxynucleotide
/5Phos/AAAAAAAAAA/i2MOErA/*/i2MOErA/*/i2MOErA/*/i2MOErA/*/i2MOErA/*/i2MO
ErA/*/3ddC/
Where 3ddC is

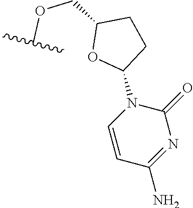 [SMILES: *OC[C@@H]1CC[C@H](n2ccc(N)nc2=O)O1]

SEQ ID NO: 79
PolyA-S-Maleimide-SulfoCy5
/5Phos/AAAAAAAAAAAAAAAAdA/3ThioMC3-MalCy5-D/
Where 3ThioMC3-MalCy5-D is

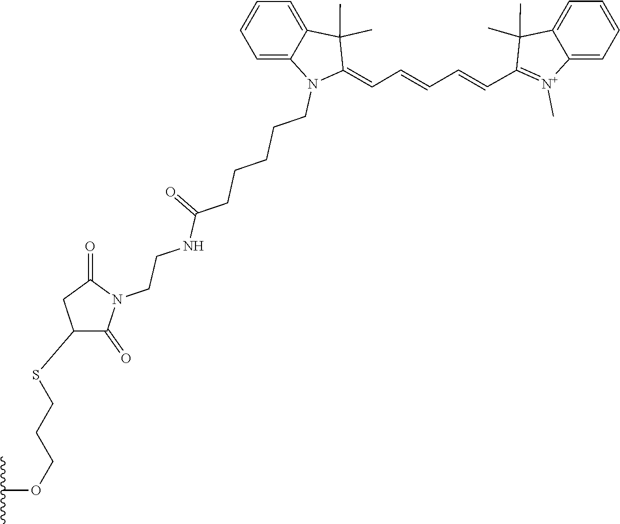 [SMILES: *OCCCSC1CC(=O)N(CCNC(=O)CCCCCN2/C(=C/C=C/C=C/C3=[N+](C)c4ccccc4C3(C)C)C(C)(C)c3ccccc32)C1=O]

SEQ ID NO: 80
PolyA-S-Maleimide-PEG19-Mal
/5Phos/AAAAAAAAAAAAAAAAdA/3ThioMC3-MalPEG19Mal/
Where 3ThioMC3-MalPEG19Mal is

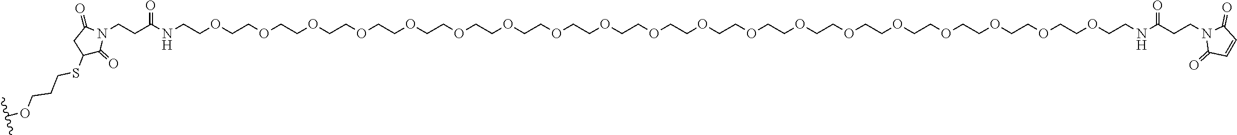 [SMILES: *OCCCSC1CC(=O)N(CCC(=O)NCCOCCOCCOCCOCCOCCOCCOCCOCCOCCOCCOCCOCCOCCOCCOCCOCCOCCOCCOCCNC(=O)CCN2C(=O)C=CC2=O)C1=O]

SEQ ID NO: 81
PolyA-S-Maleimide-PEG19-Maleimide-S-PolyA
/5Phos/AAAAAAAAAAAAAAAAdA/3ThioMC3-MalPEG19Mal-X
Where 3ThioMC3-MalPEG19Mal-X is

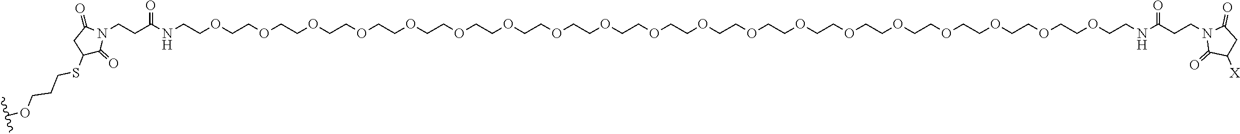

Where X is the appropriate linkage to PolyA-SH wherein the hydrogen has been removed so as
to be connected to the maleimide with the appropriate linkage
SEQ ID NO: 82
FLuc-NC-ddnt
AGGAAAUAAGAGAGAAAAGAAGAGUAAGAAGAAAUAUAAGAGCCACCAUGGAGG
ACGCCAAGAACAUCAAGAAGGGCCCCGCCCCCUUCUACCCCCUGGAGGACGGCAC
CGCCGGCGAGCAGCUGCACAAGGCCAUGAAGCGGUACGCCCUGGUGCCCGGCACC
AUCGCCUUCACCGACGCCCACAUCGAGGUGGACAUCACCUACGCCGAGUACUUCG
AGAUGAGCGUGCGGCUGGCCGAGGCCAUGAAGCGGUACGGCCUGAACACCAACCA
CCGGAUCGUGGUGUGCAGCGAGAACAGCCUGCAGUUCUUCAUGCCCGUGCUGGGC
GCCCUGUUCAUCGGCGUGGCCGUGGCCCCCGCCAACGACAUCUACAACGAGCGGG
AGCUGCUGAACAGCAUGGGCAUCAGCCAGCCCACCGUGGUGUUCGUGAGCAAGAA
GGGCCUGCAGAAGAUCCUGAACGUGCAGAAGAAGCUGCCCAUCAUCCAGAAGAUC
AUCAUCAUGGACAGCAAGACCGACUACCAGGGCUUCCAGAGCAUGUACACCUUCG
UGACCAGCCACCUGCCCCCCGGCUUCAACGAGUACGACUUCGUGCCCGAGAGCUU
CGACCGGGACAAGACCAUCGCCCUGAUCAUGAACAGCAGCGGCAGCACCGGCCUG
CCCAAGGGCGUGGCCCUGCCCCACCGGACCGCCUGCGUGCGGUUCAGCCACGCCC
GGGACCCCAUCUUCGGCAACCAGAUCAUCCCCGACACCGCCAUCCUGAGCGUGGU
GCCCUUCCACCACGGCUUCGGCAUGUUCACCACCCUGGGCUACCUGAUCUGCGGC
UUCCGGGUGGUGCUGAUGUACCGGUUCGAGGAGGAGCUGUUCCUGCGGAGCCUG
CAGGACUACAAGAUCCAGAGCGCCCUGCUGGUGCCCACCCUGUUCAGCUUCUUCG
CCAAGAGCACCCUGAUCGACAAGUACGACCUGAGCAACCUGCACGAGAUCGCCAG
CGGCGGCGCCCCCCUGAGCAAGGAGGUGGGCGAGGCCGUGGCCAAGCGGUUCCAC
CUGCCCGGCAUCCGGCAGGGCUACGGCCUGACCGAGACCACCAGCGCCAUCCUGA
UCACCCCCGAGGGCGACGACAAGCCCGGCGCCGUGGGCAAGGUGGUGCCCUUCUU
CGAGGCCAAGGUGGUGGACCUGGACACCGGCAAGACCCUGGGCGUGAACCAGCGG
GGCGAGCUGUGCGUGCGGGGCCCCAUGAUCAUGAGCGGCUACGUGAACAACCCCG
AGGCCACCAACGCCCUGAUCGACAAGGACGGCUGGCUGCACAGCGGCGACAUCGC
CUACUGGGACGAGGACGAGCACUUCUUCAUCGUGGACCGGCUGAAGAGCCUGAUC
AAGUACAAGGGCUACCAGGUGGCCCCCGCCGAGCUGGAGAGCAUCCUGCUGCAGC
ACCCCAACAUCUUCGACGCCGGCGUGGCCGGCCUGCCCGACGACGACGCCGGCGA
GCUGCCCGCCGCCGUGGUGGUGCUGGAGCACGGCAAGACCAUGACCGAGAAGGAG
AUCGUGGACUACGUGGCCAGCCAGGUGACCACCGCCAAGAAGCUGCGGGGCGGCG
UGGUGUUCGUGGACGAGGUGCCCAAGGGCCUGACCGGCAAGCUGGACGCCCGGAA
GAUCCGGGAGAUCCUGAUCAAGGCCAAGAAGGGCGGCAAGAUCGCCGUGUAAGC
UGCCUUCUGCGGGGCUUGCCUUCUGGCCAUGCCCUUCUUCUCUCCCUUGCACCUG
UACCUCUUGGUCUUUGAAUAAAGCCUGAGUAGGAAGAAAAAAAAAAAAAAAAAA
AAAAAAAAAAAAAAAAAAAAAAAAAAAAAAAAAAAAAAAAAAAAAAAAAAAAAA
AAAAAAAAAAAAAAAAAAAAAAAAAAAAAAAAAAAAAAAAAAAAAAAAAAAAAA
AAAAA/i2MOErA/*/i2MOErA/*/i2MOErA/*/i2MOErA/*/i2MOErA/*/i2MOErA/*/3ddC/
SEQ ID NO: 83
FLuc-NC-SR
AGGAAAUAAGAGAGAAAAGAAGAGUAAGAAGAAAUAUAAGAGCCACCAUGGAGG
ACGCCAAGAACAUCAAGAAGGGCCCCGCCCCCUUCUACCCCCUGGAGGACGGCAC
CGCCGGCGAGCAGCUGCACAAGGCCAUGAAGCGGUACGCCCUGGUGCCCGGCACC
AUCGCCUUCACCGACGCCCACAUCGAGGUGGACAUCACCUACGCCGAGUACUUCG
AGAUGAGCGUGCGGCUGGCCGAGGCCAUGAAGCGGUACGGCCUGAACACCAACCA
CCGGAUCGUGGUGUGCAGCGAGAACAGCCUGCAGUUCUUCAUGCCCGUGCUGGGC
GCCCUGUUCAUCGGCGUGGCCGUGGCCCCCGCCAACGACAUCUACAACGAGCGGG
AGCUGCUGAACAGCAUGGGCAUCAGCCAGCCCACCGUGGUGUUCGUGAGCAAGAA
GGGCCUGCAGAAGAUCCUGAACGUGCAGAAGAAGCUGCCCAUCAUCCAGAAGAUC
AUCAUCAUGGACAGCAAGACCGACUACCAGGGCUUCCAGAGCAUGUACACCUUCG
UGACCAGCCACCUGCCCCCCGGCUUCAACGAGUACGACUUCGUGCCCGAGAGCUU
CGACCGGGACAAGACCAUCGCCCUGAUCAUGAACAGCAGCGGCAGCACCGGCCUG
CCCAAGGGCGUGGCCCUGCCCCACCGGACCGCCUGCGUGCGGUUCAGCCACGCCC
GGGACCCCAUCUUCGGCAACCAGAUCAUCCCCGACACCGCCAUCCUGAGCGUGGU
GCCCUUCCACCACGGCUUCGGCAUGUUCACCACCCUGGGCUACCUGAUCUGCGGC
UUCCGGGUGGUGCUGAUGUACCGGUUCGAGGAGGAGCUGUUCCUGCGGAGCCUG
CAGGACUACAAGAUCCAGAGCGCCCUGCUGGUGCCCACCCUGUUCAGCUUCUUCG
CCAAGAGCACCCUGAUCGACAAGUACGACCUGAGCAACCUGCACGAGAUCGCCAG
CGGCGGCGCCCCCCUGAGCAAGGAGGUGGGCGAGGCCGUGGCCAAGCGGUUCCAC
CUGCCCGGCAUCCGGCAGGGCUACGGCCUGACCGAGACCACCAGCGCCAUCCUGA
UCACCCCCGAGGGCGACGACAAGCCCGGCGCCGUGGGCAAGGUGGUGCCCUUCUU
CGAGGCCAAGGUGGUGGACCUGGACACCGGCAAGACCCUGGGCGUGAACCAGCGG
GGCGAGCUGUGCGUGCGGGGCCCCAUGAUCAUGAGCGGCUACGUGAACAACCCCG
AGGCCACCAACGCCCUGAUCGACAAGGACGGCUGGCUGCACAGCGGCGACAUCGC
CUACUGGGACGAGGACGAGCACUUCUUCAUCGUGGACCGGCUGAAGAGCCUGAUC
AAGUACAAGGGCUACCAGGUGGCCCCCGCCGAGCUGGAGAGCAUCCUGCUGCAGC
ACCCCAACAUCUUCGACGCCGGCGUGGCCGGCCUGCCCGACGACGACGCCGGCGA
GCUGCCCGCCGCCGUGGUGGUGCUGGAGCACGGCAAGACCAUGACCGAGAAGGAG
AUCGUGGACUACGUGGCCAGCCAGGUGACCACCGCCAAGAAGCUGCGGGGCGGCG
UGGUGUUCGUGGACGAGGUGCCCAAGGGCCUGACCGGCAAGCUGGACGCCCGGAA
GAUCCGGGAGAUCCUGAUCAAGGCCAAGAAGGGCGGCAAGAUCGCCGUGUAAGC
UGCCUUCUGCGGGGCUUGCCUUCUGGCCAUGCCCUUCUUCUCUCCCUUGCACCUG
UACCUCUUGGUCUUUGAAUAAAGCCUGAGUAGGAAGAAAAAAAAAAAAAAAAAA
AAAAAAAAAAAAAAAAAAAAAAAAAAAAAAAAAAAAAAAAAAAAAAAAAAAAAA
AAAAAAAAAAAAAAAAAAAAAAAAAAAAAAAAAAAAAAAAAAAAAAAAAAAAAA
AAAAA/i2MOErA/*/i2MOErA/*/i2MOErA/*/i2MOErA/*/i2MOErA/*/i2MOErA/*/3ThioMC3
-D/
SEQ ID NO: 84
FLuc-NC-SH
AGGAAAUAAGAGAGAAAAGAAGAGUAAGAAGAAAUAUAAGAGCCACCAUGGAGG
ACGCCAAGAACAUCAAGAAGGGCCCCGCCCCCUUCUACCCCCUGGAGGACGGCAC
CGCCGGCGAGCAGCUGCACAAGGCCAUGAAGCGGUACGCCCUGGUGCCCGGCACC
AUCGCCUUCACCGACGCCCACAUCGAGGUGGACAUCACCUACGCCGAGUACUUCG
AGAUGAGCGUGCGGCUGGCCGAGGCCAUGAAGCGGUACGGCCUGAACACCAACCA
CCGGAUCGUGGUGUGCAGCGAGAACAGCCUGCAGUUCUUCAUGCCCGUGCUGGGC
GCCCUGUUCAUCGGCGUGGCCGUGGCCCCCGCCAACGACAUCUACAACGAGCGGG
AGCUGCUGAACAGCAUGGGCAUCAGCCAGCCCACCGUGGUGUUCGUGAGCAAGAA
GGGCCUGCAGAAGAUCCUGAACGUGCAGAAGAAGCUGCCCAUCAUCCAGAAGAUC
AUCAUCAUGGACAGCAAGACCGACUACCAGGGCUUCCAGAGCAUGUACACCUUCG
UGACCAGCCACCUGCCCCCCGGCUUCAACGAGUACGACUUCGUGCCCGAGAGCUU
CGACCGGGACAAGACCAUCGCCCUGAUCAUGAACAGCAGCGGCAGCACCGGCCUG
CCCAAGGGCGUGGCCCUGCCCCACCGGACCGCCUGCGUGCGGUUCAGCCACGCCC
GGGACCCCAUCUUCGGCAACCAGAUCAUCCCCGACACCGCCAUCCUGAGCGUGGU
GCCCUUCCACCACGGCUUCGGCAUGUUCACCACCCUGGGCUACCUGAUCUGCGGC
UUCCGGGUGGUGCUGAUGUACCGGUUCGAGGAGGAGCUGUUCCUGCGGAGCCUG
CAGGACUACAAGAUCCAGAGCGCCCUGCUGGUGCCCACCCUGUUCAGCUUCUUCG
CCAAGAGCACCCUGAUCGACAAGUACGACCUGAGCAACCUGCACGAGAUCGCCAG
CGGCGGCGCCCCCCUGAGCAAGGAGGUGGGCGAGGCCGUGGCCAAGCGGUUCCAC
CUGCCCGGCAUCCGGCAGGGCUACGGCCUGACCGAGACCACCAGCGCCAUCCUGA
UCACCCCCGAGGGCGACGACAAGCCCGGCGCCGUGGGCAAGGUGGUGCCCUUCUU
CGAGGCCAAGGUGGUGGACCUGGACACCGGCAAGACCCUGGGCGUGAACCAGCGG
GGCGAGCUGUGCGUGCGGGGCCCCAUGAUCAUGAGCGGCUACGUGAACAACCCCG
AGGCCACCAACGCCCUGAUCGACAAGGACGGCUGGCUGCACAGCGGCGACAUCGC
CUACUGGGACGAGGACGAGCACUUCUUCAUCGUGGACCGGCUGAAGAGCCUGAUC
AAGUACAAGGGCUACCAGGUGGCCCCCGCCGAGCUGGAGAGCAUCCUGCUGCAGC
ACCCCAACAUCUUCGACGCCGGCGUGGCCGGCCUGCCCGACGACGACGCCGGCGA
GCUGCCCGCCGCCGUGGUGGUGCUGGAGCACGGCAAGACCAUGACCGAGAAGGAG
AUCGUGGACUACGUGGCCAGCCAGGUGACCACCGCCAAGAAGCUGCGGGGCGGCG
UGGUGUUCGUGGACGAGGUGCCCAAGGGCCUGACCGGCAAGCUGGACGCCCGGAA
GAUCCGGGAGAUCCUGAUCAAGGCCAAGAAGGGCGGCAAGAUCGCCGUGUAAGC
UGCCUUCUGCGGGGCUUGCCUUCUGGCCAUGCCCUUCUUCUCUCCCUUGCACCUG
UACCUCUUGGUCUUUGAAUAAAGCCUGAGUAGGAAGAAAAAAAAAAAAAAAAAA
AAAAAAAAAAAAAAAAAAAAAAAAAAAAAAAAAAAAAAAAAAAAAAAAAAAAAA
AAAAAAAAAAAAAAAAAAAAAAAAAAAAAAAAAAAAAAAAAAAAAAAAAAAAAA
AAAAA/i2MOErA/*/i2MOErA/*/i2MOErA/*/i2MOErA/*/i2MOErA/*/i2MOErA/*/3ThioMC3
Reduced-D/
SEQ ID NO: 85
eGFP-S-Maleimide-SulfoCy5
AGGAAAUAAGAGAGAAAAGAAGAGUAAGAAGAAAUAUAAGAGCCACCAUGGUGA
GCAAGGGCGAGGAGCUGUUCACCGGGGUGGUGCCCAUCCUGGUCGAGCUGGACGG
CGACGUAAACGGCCACAAGUUCAGCGUGUCCGGCGAGGGCGAGGGCGAUGCCACC
UACGGCAAGCUGACCCUGAAGUUCAUCUGCACCACCGGCAAGCUGCCCGUGCCCU
GGCCCACCCUCGUGACCACCCUGACCUACGGCGUGCAGUGCUUCAGCCGCUACCC
CGACCACAUGAAGCAGCACGACUUCUUCAAGUCCGCCAUGCCCGAAGGCUACGUC
CAGGAGCGCACCAUCUUCUUCAAGGACGACGGCAACUACAAGACCCGCGCCGAGG
UGAAGUUCGAGGGCGACACCCUGGUGAACCGCAUCGAGCUGAAGGGCAUCGACUU
CAAGGAGGACGGCAACAUCCUGGGGCACAAGCUGGAGUACAACUACAACAGCCAC
AACGUCUAUAUCAUGGCCGACAAGCAGAAGAACGGCAUCAAGGUGAACUUCAAG
AUCCGCCACAACAUCGAGGACGGCAGCGUGCAGCUCGCCGACCACUACCAGCAGA
ACACCCCCAUCGGCGACGGCCCCGUGCUGCUGCCCGACAACCACUACCUGAGCACC
CAGUCCGCCCUGAGCAAAGACCCCAACGAGAAGCGCGAUCACAUGGUCCUGCUGG
AGUUCGUGACCGCCGCCGGGAUCACUCUCGGCAUGGACGAGCUGUACAAGUAAGC
UGCCUUCUGCGGGGCUUGCCUUCUGGCCAUGCCCUUCUUCUCUCCCUUGCACCUG
UACCUCUUGGUCUUUGAAUAAAGCCUGAGUAGGAAGAAAAAAAAAAAAAAAAAA
AAAAAAAAAAAAAAAAAAAAAAAAAAAAAAAAAAAAAAAAAAAAAAAAAAAAAA
AAAAAAAAAAAAAAAAAAAAAAAAAAAAAAAAAAAAAAAAAAAAAAAAAAAAAA
AAAAAAAAAAAdA/3ThioMC3-MalCy5-D/
SEQ ID NO: 86
mCherry-S-Maleimide-SulfoCy5
AGGAAAUAAGAGAGAAAAGAAGAGUAAGAAGAAAUAUAAGAGCCACCAUGGUGA
GCAAGGGCGAGGAGGACAACAUGGCCAUCAUCAAGGAGUUCAUGCGGUUCAAGG
UGCACAUGGAGGGCAGCGUGAACGGCCACGAGUUCGAGAUCGAGGGCGAGGGCG
AGGGCCGGCCCUACGAGGGCACCCAGACCGCCAAGCUGAAGGUGACCAAGGGCGG
CCCCCUGCCCUUCGCCUGGGACAUCCUGAGCCCCCAGUUCAUGUACGGCAGCAAG
GCCUACGUGAAGCACCCCGCCGACAUCCCCGACUACCUGAAGCUGAGCUUCCCCG
AGGGCUUCAAGUGGGAGCGGGUGAUGAACUUCGAGGACGGCGGCGUGGUGACCG
UGACCCAGGACAGCAGCCUGCAGGACGGCGAGUUCAUCUACAAGGUGAAGCUGCG
GGGCACCAACUUCCCCAGCGACGGCCCCGUGAUGCAGAAGAAGACCAUGGGCUGG
GAGGCCAGCAGCGAGCGGAUGUACCCCGAGGACGGCGCCCUGAAGGGCGAGAUCA
AGCAGCGGCUGAAGCUGAAGGACGGCGGCCACUACGACGCCGAGGUGAAGACCAC
CUACAAGGCCAAGAAGCCCGUGCAGCUGCCCGGCGCCUACAACGUGAACAUCAAG
CUGGACAUCACCAGCCACAACGAGGACUACACCAUCGUGGAGCAGUACGAGCGGG
CCGAGGGCCGGCACAGCACCGGCGGCAUGGACGAGCUGUACAAGAGCGGCAACUG
AGCUGCCUUCUGCGGGGCUUGCCUUCUGGCCAUGCCCUUCUUCUCUCCCUUGCAC
CUGUACCUCUUGGUCUUUGAAUAAAGCCUGAGUAGGAAGAAAAAAAAAAAAAAA
AAAAAAAAAAAAAAAAAAAAAAAAAAAAAAAAAAAAAAAAAAAAAAAAAAAAAA
AAAAAAAAAAAAAAAAAAAAAAAAAAAAAAAAAAAAAAAAAAAAAAAAAAAAAA
AAAAAAAAAAAAAAdA/3ThioMC3-MalCy5-D/
SEQ ID NO: 87
FLuc-S-Maleimide-SulfoCy5
AGGAAAUAAGAGAGAAAAGAAGAGUAAGAAGAAAUAUAAGAGCCACCAUGGAGG
ACGCCAAGAACAUCAAGAAGGGCCCCGCCCCCUUCUACCCCCUGGAGGACGGCAC
CGCCGGCGAGCAGCUGCACAAGGCCAUGAAGCGGUACGCCCUGGUGCCCGGCACC
AUCGCCUUCACCGACGCCCACAUCGAGGUGGACAUCACCUACGCCGAGUACUUCG
AGAUGAGCGUGCGGCUGGCCGAGGCCAUGAAGCGGUACGGCCUGAACACCAACCA
CCGGAUCGUGGUGUGCAGCGAGAACAGCCUGCAGUUCUUCAUGCCCGUGCUGGGC
GCCCUGUUCAUCGGCGUGGCCGUGGCCCCCGCCAACGACAUCUACAACGAGCGGG
AGCUGCUGAACAGCAUGGGCAUCAGCCAGCCCACCGUGGUGUUCGUGAGCAAGAA
GGGCCUGCAGAAGAUCCUGAACGUGCAGAAGAAGCUGCCCAUCAUCCAGAAGAUC
AUCAUCAUGGACAGCAAGACCGACUACCAGGGCUUCCAGAGCAUGUACACCUUCG
UGACCAGCCACCUGCCCCCCGGCUUCAACGAGUACGACUUCGUGCCCGAGAGCUU
CGACCGGGACAAGACCAUCGCCCUGAUCAUGAACAGCAGCGGCAGCACCGGCCUG
CCCAAGGGCGUGGCCCUGCCCCACCGGACCGCCUGCGUGCGGUUCAGCCACGCCC
GGGACCCCAUCUUCGGCAACCAGAUCAUCCCCGACACCGCCAUCCUGAGCGUGGU
GCCCUUCCACCACGGCUUCGGCAUGUUCACCACCCUGGGCUACCUGAUCUGCGGC
UUCCGGGUGGUGCUGAUGUACCGGUUCGAGGAGGAGCUGUUCCUGCGGAGCCUG
CAGGACUACAAGAUCCAGAGCGCCCUGCUGGUGCCCACCCUGUUCAGCUUCUUCG
CCAAGAGCACCCUGAUCGACAAGUACGACCUGAGCAACCUGCACGAGAUCGCCAG
CGGCGGCGCCCCCCUGAGCAAGGAGGUGGGCGAGGCCGUGGCCAAGCGGUUCCAC
CUGCCCGGCAUCCGGCAGGGCUACGGCCUGACCGAGACCACCAGCGCCAUCCUGA
UCACCCCCGAGGGCGACGACAAGCCCGGCGCCGUGGGCAAGGUGGUGCCCUUCUU
CGAGGCCAAGGUGGUGGACCUGGACACCGGCAAGACCCUGGGCGUGAACCAGCGG
GGCGAGCUGUGCGUGCGGGGCCCCAUGAUCAUGAGCGGCUACGUGAACAACCCCG
AGGCCACCAACGCCCUGAUCGACAAGGACGGCUGGCUGCACAGCGGCGACAUCGC
CUACUGGGACGAGGACGAGCACUUCUUCAUCGUGGACCGGCUGAAGAGCCUGAUC
AAGUACAAGGGCUACCAGGUGGCCCCCGCCGAGCUGGAGAGCAUCCUGCUGCAGC
ACCCCAACAUCUUCGACGCCGGCGUGGCCGGCCUGCCCGACGACGACGCCGGCGA
GCUGCCCGCCGCCGUGGUGGUGCUGGAGCACGGCAAGACCAUGACCGAGAAGGAG
AUCGUGGACUACGUGGCCAGCCAGGUGACCACCGCCAAGAAGCUGCGGGGCGGCG
UGGUGUUCGUGGACGAGGUGCCCAAGGGCCUGACCGGCAAGCUGGACGCCCGGAA
GAUCCGGGAGAUCCUGAUCAAGGCCAAGAAGGGCGGCAAGAUCGCCGUGUGAGC
UGCCUUCUGCGGGGCUUGCCUUCUGGCCAUGCCCUUCUUCUCUCCCUUGCACCUG
UACCUCUUGGUCUUUGAAUAAAGCCUGAGUAGGAAGAAAAAAAAAAAAAAAAAA
AAAAAAAAAAAAAAAAAAAAAAAAAAAAAAAAAAAAAAAAAAAAAAAAAAAAAA
AAAAAAAAAAAAAAAAAAAAAAAAAAAAAAAAAAAAAAAAAAAAAAAAAAAAAA
AAAAAAAAAAAdA/3ThioMC3-MalCy5-D/
SEQ ID NO: 88
FLuc-S-Maleimide-GalNAc
AGGAAAUAAGAGAGAAAAGAAGAGUAAGAAGAAAUAUAAGAGCCACCAUGGAGG
ACGCCAAGAACAUCAAGAAGGGCCCCGCCCCCUUCUACCCCCUGGAGGACGGCAC
CGCCGGCGAGCAGCUGCACAAGGCCAUGAAGCGGUACGCCCUGGUGCCCGGCACC
AUCGCCUUCACCGACGCCCACAUCGAGGUGGACAUCACCUACGCCGAGUACUUCG
AGAUGAGCGUGCGGCUGGCCGAGGCCAUGAAGCGGUACGGCCUGAACACCAACCA
CCGGAUCGUGGUGUGCAGCGAGAACAGCCUGCAGUUCUUCAUGCCCGUGCUGGGC
GCCCUGUUCAUCGGCGUGGCCGUGGCCCCCGCCAACGACAUCUACAACGAGCGGG
AGCUGCUGAACAGCAUGGGCAUCAGCCAGCCCACCGUGGUGUUCGUGAGCAAGAA
GGGCCUGCAGAAGAUCCUGAACGUGCAGAAGAAGCUGCCCAUCAUCCAGAAGAUC
AUCAUCAUGGACAGCAAGACCGACUACCAGGGCUUCCAGAGCAUGUACACCUUCG
UGACCAGCCACCUGCCCCCCGGCUUCAACGAGUACGACUUCGUGCCCGAGAGCUU
CGACCGGGACAAGACCAUCGCCCUGAUCAUGAACAGCAGCGGCAGCACCGGCCUG
CCCAAGGGCGUGGCCCUGCCCCACCGGACCGCCUGCGUGCGGUUCAGCCACGCCC
GGGACCCCAUCUUCGGCAACCAGAUCAUCCCCGACACCGCCAUCCUGAGCGUGGU
GCCCUUCCACCACGGCUUCGGCAUGUUCACCACCCUGGGCUACCUGAUCUGCGGC
UUCCGGGUGGUGCUGAUGUACCGGUUCGAGGAGGAGCUGUUCCUGCGGAGCCUG
CAGGACUACAAGAUCCAGAGCGCCCUGCUGGUGCCCACCCUGUUCAGCUUCUUCG
CCAAGAGCACCCUGAUCGACAAGUACGACCUGAGCAACCUGCACGAGAUCGCCAG
CGGCGGCGCCCCCCUGAGCAAGGAGGUGGGCGAGGCCGUGGCCAAGCGGUUCCAC
CUGCCCGGCAUCCGGCAGGGCUACGGCCUGACCGAGACCACCAGCGCCAUCCUGA
UCACCCCCGAGGGCGACGACAAGCCCGGCGCCGUGGGCAAGGUGGUGCCCUUCUU
CGAGGCCAAGGUGGUGGACCUGGACACCGGCAAGACCCUGGGCGUGAACCAGCGG
GGCGAGCUGUGCGUGCGGGGCCCCAUGAUCAUGAGCGGCUACGUGAACAACCCCG
AGGCCACCAACGCCCUGAUCGACAAGGACGGCUGGCUGCACAGCGGCGACAUCGC
CUACUGGGACGAGGACGAGCACUUCUUCAUCGUGGACCGGCUGAAGAGCCUGAUC
AAGUACAAGGGCUACCAGGUGGCCCCCGCCGAGCUGGAGAGCAUCCUGCUGCAGC
ACCCCAACAUCUUCGACGCCGGCGUGGCCGGCCUGCCCGACGACGACGCCGGCGA
GCUGCCCGCCGCCGUGGUGGUGCUGGAGCACGGCAAGACCAUGACCGAGAAGGAG
AUCGUGGACUACGUGGCCAGCCAGGUGACCACCGCCAAGAAGCUGCGGGGCGGCG
UGGUGUUCGUGGACGAGGUGCCCAAGGGCCUGACCGGCAAGCUGGACGCCCGGAA
GAUCCGGGAGAUCCUGAUCAAGGCCAAGAAGGGCGGCAAGAUCGCCGUGUGAGC
UGCCUUCUGCGGGGCUUGCCUUCUGGCCAUGCCCUUCUUCUCUCCCUUGCACCUG
UACCUCUUGGUCUUUGAAUAAAGCCUGAGUAGGAAGAAAAAAAAAAAAAAAAAA
AAAAAAAAAAAAAAAAAAAAAAAAAAAAAAAAAAAAAAAAAAAAAAAAAAAAAA
AAAAAAAAAAAAAAAAAAAAAAAAAAAAAAAAAAAAAAAAAAAAAAAAAAAAAA
AAAAAAAAAAAdA/3ThioMC3-GalNAc-D/
Where 3ThioMC3-GalNAc-D is

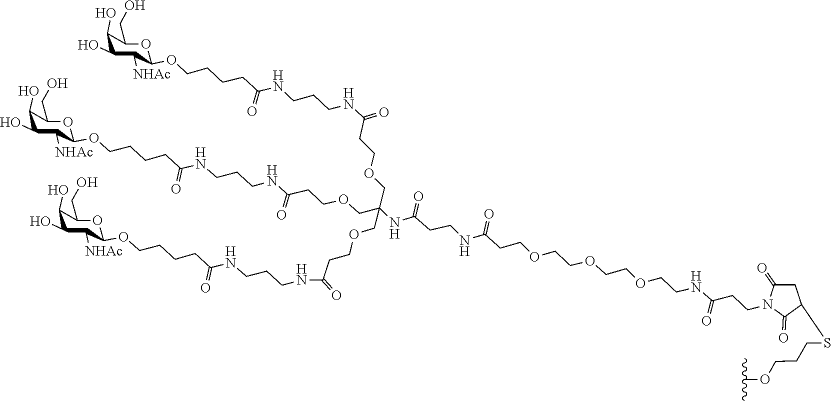

SEQ ID NO: 89
FLuc-NC-S-Maleimide-GalNAc
AGGAAAUAAGAGAGAAAAGAAGAGUAAGAAGAAAUAUAAGAGCCACCAUGGAGG
ACGCCAAGAACAUCAAGAAGGGCCCCGCCCCCUUCUACCCCCUGGAGGACGGCAC
CGCCGGCGAGCAGCUGCACAAGGCCAUGAAGCGGUACGCCCUGGUGCCCGGCACC
AUCGCCUUCACCGACGCCCACAUCGAGGUGGACAUCACCUACGCCGAGUACUUCG
AGAUGAGCGUGCGGCUGGCCGAGGCCAUGAAGCGGUACGGCCUGAACACCAACCA
CCGGAUCGUGGUGUGCAGCGAGAACAGCCUGCAGUUCUUCAUGCCCGUGCUGGGC
GCCCUGUUCAUCGGCGUGGCCGUGGCCCCCGCCAACGACAUCUACAACGAGCGGG
AGCUGCUGAACAGCAUGGGCAUCAGCCAGCCCACCGUGGUGUUCGUGAGCAAGAA
GGGCCUGCAGAAGAUCCUGAACGUGCAGAAGAAGCUGCCCAUCAUCCAGAAGAUC
AUCAUCAUGGACAGCAAGACCGACUACCAGGGCUUCCAGAGCAUGUACACCUUCG
UGACCAGCCACCUGCCCCCCGGCUUCAACGAGUACGACUUCGUGCCCGAGAGCUU
CGACCGGGACAAGACCAUCGCCCUGAUCAUGAACAGCAGCGGCAGCACCGGCCUG
CCCAAGGGCGUGGCCCUGCCCCACCGGACCGCCUGCGUGCGGUUCAGCCACGCCC
GGGACCCCAUCUUCGGCAACCAGAUCAUCCCCGACACCGCCAUCCUGAGCGUGGU
GCCCUUCCACCACGGCUUCGGCAUGUUCACCACCCUGGGCUACCUGAUCUGCGGC
UUCCGGGUGGUGCUGAUGUACCGGUUCGAGGAGGAGCUGUUCCUGCGGAGCCUG
CAGGACUACAAGAUCCAGAGCGCCCUGCUGGUGCCCACCCUGUUCAGCUUCUUCG
CCAAGAGCACCCUGAUCGACAAGUACGACCUGAGCAACCUGCACGAGAUCGCCAG
CGGCGGCGCCCCCCUGAGCAAGGAGGUGGGCGAGGCCGUGGCCAAGCGGUUCCAC
CUGCCCGGCAUCCGGCAGGGCUACGGCCUGACCGAGACCACCAGCGCCAUCCUGA
UCACCCCCGAGGGCGACGACAAGCCCGGCGCCGUGGGCAAGGUGGUGCCCUUCUU
CGAGGCCAAGGUGGUGGACCUGGACACCGGCAAGACCCUGGGCGUGAACCAGCGG
GGCGAGCUGUGCGUGCGGGGCCCCAUGAUCAUGAGCGGCUACGUGAACAACCCCG
AGGCCACCAACGCCCUGAUCGACAAGGACGGCUGGCUGCACAGCGGCGACAUCGC
CUACUGGGACGAGGACGAGCACUUCUUCAUCGUGGACCGGCUGAAGAGCCUGAUC
AAGUACAAGGGCUACCAGGUGGCCCCCGCCGAGCUGGAGAGCAUCCUGCUGCAGC
ACCCCAACAUCUUCGACGCCGGCGUGGCCGGCCUGCCCGACGACGACGCCGGCGA
GCUGCCCGCCGCCGUGGUGGUGCUGGAGCACGGCAAGACCAUGACCGAGAAGGAG
AUCGUGGACUACGUGGCCAGCCAGGUGACCACCGCCAAGAAGCUGCGGGGCGGCG
UGGUGUUCGUGGACGAGGUGCCCAAGGGCCUGACCGGCAAGCUGGACGCCCGGAA
GAUCCGGGAGAUCCUGAUCAAGGCCAAGAAGGGCGGCAAGAUCGCCGUGUGAGC
UGCCUUCUGCGGGGCUUGCCUUCUGGCCAUGCCCUUCUUCUCUCCCUUGCACCUG
UACCUCUUGGUCUUUGAAUAAAGCCUGAGUAGGAAGAAAAAAAAAAAAAAAAAA
AAAAAAAAAAAAAAAAAAAAAAAAAAAAAAAAAAAAAAAAAAAAAAAAAAAAAA
AAAAAAAAAAAAAAAAAAAAAAAAAAAAAAAAAAAAAAAAAAAAAAAAAAAAAA
AAAAA/i2MOErA/*/i2MOErA/*/i2MOErA/*/i2MOErA/*/i2MOErA/*/i2MOErA/*/3ThioMC3-
GalNAc-D/
SEQ ID NO: 90
eGFP-S-Maleimide-PEG19-Mal
AGGAAAUAAGAGAGAAAAGAAGAGUAAGAAGAAAUAUAAGAGCCACCAUGGUGA
GCAAGGGCGAGGAGCUGUUCACCGGGGUGGUGCCCAUCCUGGUCGAGCUGGACGG
CGACGUAAACGGCCACAAGUUCAGCGUGUCCGGCGAGGGCGAGGGCGAUGCCACC
UACGGCAAGCUGACCCUGAAGUUCAUCUGCACCACCGGCAAGCUGCCCGUGCCCU
GGCCCACCCUCGUGACCACCCUGACCUACGGCGUGCAGUGCUUCAGCCGCUACCC
CGACCACAUGAAGCAGCACGACUUCUUCAAGUCCGCCAUGCCCGAAGGCUACGUC
CAGGAGCGCACCAUCUUCUUCAAGGACGACGGCAACUACAAGACCCGCGCCGAGG
UGAAGUUCGAGGGCGACACCCUGGUGAACCGCAUCGAGCUGAAGGGCAUCGACUU
CAAGGAGGACGGCAACAUCCUGGGGCACAAGCUGGAGUACAACUACAACAGCCAC
AACGUCUAUAUCAUGGCCGACAAGCAGAAGAACGGCAUCAAGGUGAACUUCAAG
AUCCGCCACAACAUCGAGGACGGCAGCGUGCAGCUCGCCGACCACUACCAGCAGA
ACACCCCCAUCGGCGACGGCCCCGUGCUGCUGCCCGACAACCACUACCUGAGCACC
CAGUCCGCCCUGAGCAAAGACCCCAACGAGAAGCGCGAUCACAUGGUCCUGCUGG
AGUUCGUGACCGCCGCCGGGAUCACUCUCGGCAUGGACGAGCUGUACAAGUAAGC
UGCCUUCUGCGGGGCUUGCCUUCUGGCCAUGCCCUUCUUCUCUCCCUUGCACCUG
UACCUCUUGGUCUUUGAAUAAAGCCUGAGUAGGAAGAAAAAAAAAAAAAAAAAA
AAAAAAAAAAAAAAAAAAAAAAAAAAAAAAAAAAAAAAAAAAAAAAAAAAAAAA
AAAAAAAAAAAAAAAAAAAAAAAAAAAAAAAAAAAAAAAAAAAAAAAAAAAAAA
AAAAAAAAAAAdA/3ThioMC3-MalPEG19Mal/
SEQ ID NO: 91
mCherry-S-Maleimide-PEG19-Mal
AGGAAAUAAGAGAGAAAAGAAGAGUAAGAAGAAAUAUAAGAGCCACCAUGGUGA
GCAAGGGCGAGGAGGACAACAUGGCCAUCAUCAAGGAGUUCAUGCGGUUCAAGG
UGCACAUGGAGGGCAGCGUGAACGGCCACGAGUUCGAGAUCGAGGGCGAGGGCG
AGGGCCGGCCCUACGAGGGCACCCAGACCGCCAAGCUGAAGGUGACCAAGGGCGG
CCCCCUGCCCUUCGCCUGGGACAUCCUGAGCCCCCAGUUCAUGUACGGCAGCAAG
GCCUACGUGAAGCACCCCGCCGACAUCCCCGACUACCUGAAGCUGAGCUUCCCCG
AGGGCUUCAAGUGGGAGCGGGUGAUGAACUUCGAGGACGGCGGCGUGGUGACCG
UGACCCAGGACAGCAGCCUGCAGGACGGCGAGUUCAUCUACAAGGUGAAGCUGCG
GGGCACCAACUUCCCCAGCGACGGCCCCGUGAUGCAGAAGAAGACCAUGGGCUGG
GAGGCCAGCAGCGAGCGGAUGUACCCCGAGGACGGCGCCCUGAAGGGCGAGAUCA
AGCAGCGGCUGAAGCUGAAGGACGGCGGCCACUACGACGCCGAGGUGAAGACCAC
CUACAAGGCCAAGAAGCCCGUGCAGCUGCCCGGCGCCUACAACGUGAACAUCAAG
CUGGACAUCACCAGCCACAACGAGGACUACACCAUCGUGGAGCAGUACGAGCGGG
CCGAGGGCCGGCACAGCACCGGCGGCAUGGACGAGCUGUACAAGAGCGGCAACUG
AGCUGCCUUCUGCGGGGCUUGCCUUCUGGCCAUGCCCUUCUUCUCUCCCUUGCAC
CUGUACCUCUUGGUCUUUGAAUAAAGCCUGAGUAGGAAGAAAAAAAAAAAAAAA
AAAAAAAAAAAAAAAAAAAAAAAAAAAAAAAAAAAAAAAAAAAAAAAAAAAAAA
AAAAAAAAAAAAAAAAAAAAAAAAAAAAAAAAAAAAAAAAAAAAAAAAAAAAAA
AAAAAAAAAAAAAAdA/3ThioMC3-MalPEG19Mal/
SEQ ID NO: 92
FLuc-S-Maleimide-PEG19-Mal
AGGAAAUAAGAGAGAAAAGAAGAGUAAGAAGAAAUAUAAGAGCCACCAUGGAGG
ACGCCAAGAACAUCAAGAAGGGCCCCGCCCCCUUCUACCCCCUGGAGGACGGCAC
CGCCGGCGAGCAGCUGCACAAGGCCAUGAAGCGGUACGCCCUGGUGCCCGGCACC
AUCGCCUUCACCGACGCCCACAUCGAGGUGGACAUCACCUACGCCGAGUACUUCG
AGAUGAGCGUGCGGCUGGCCGAGGCCAUGAAGCGGUACGGCCUGAACACCAACCA
CCGGAUCGUGGUGUGCAGCGAGAACAGCCUGCAGUUCUUCAUGCCCGUGCUGGGC
GCCCUGUUCAUCGGCGUGGCCGUGGCCCCCGCCAACGACAUCUACAACGAGCGGG
AGCUGCUGAACAGCAUGGGCAUCAGCCAGCCCACCGUGGUGUUCGUGAGCAAGAA
GGGCCUGCAGAAGAUCCUGAACGUGCAGAAGAAGCUGCCCAUCAUCCAGAAGAUC
AUCAUCAUGGACAGCAAGACCGACUACCAGGGCUUCCAGAGCAUGUACACCUUCG
UGACCAGCCACCUGCCCCCCGGCUUCAACGAGUACGACUUCGUGCCCGAGAGCUU
CGACCGGGACAAGACCAUCGCCCUGAUCAUGAACAGCAGCGGCAGCACCGGCCUG
CCCAAGGGCGUGGCCCUGCCCCACCGGACCGCCUGCGUGCGGUUCAGCCACGCCC
GGGACCCCAUCUUCGGCAACCAGAUCAUCCCCGACACCGCCAUCCUGAGCGUGGU
GCCCUUCCACCACGGCUUCGGCAUGUUCACCACCCUGGGCUACCUGAUCUGCGGC
UUCCGGGUGGUGCUGAUGUACCGGUUCGAGGAGGAGCUGUUCCUGCGGAGCCUG
CAGGACUACAAGAUCCAGAGCGCCCUGCUGGUGCCCACCCUGUUCAGCUUCUUCG
CCAAGAGCACCCUGAUCGACAAGUACGACCUGAGCAACCUGCACGAGAUCGCCAG
CGGCGGCGCCCCCCUGAGCAAGGAGGUGGGCGAGGCCGUGGCCAAGCGGUUCCAC
CUGCCCGGCAUCCGGCAGGGCUACGGCCUGACCGAGACCACCAGCGCCAUCCUGA
UCACCCCCGAGGGCGACGACAAGCCCGGCGCCGUGGGCAAGGUGGUGCCCUUCUU
CGAGGCCAAGGUGGUGGACCUGGACACCGGCAAGACCCUGGGCGUGAACCAGCGG
GGCGAGCUGUGCGUGCGGGGCCCCAUGAUCAUGAGCGGCUACGUGAACAACCCCG
AGGCCACCAACGCCCUGAUCGACAAGGACGGCUGGCUGCACAGCGGCGACAUCGC
CUACUGGGACGAGGACGAGCACUUCUUCAUCGUGGACCGGCUGAAGAGCCUGAUC
AAGUACAAGGGCUACCAGGUGGCCCCCGCCGAGCUGGAGAGCAUCCUGCUGCAGC
ACCCCAACAUCUUCGACGCCGGCGUGGCCGGCCUGCCCGACGACGACGCCGGCGA
GCUGCCCGCCGCCGUGGUGGUGCUGGAGCACGGCAAGACCAUGACCGAGAAGGAG
AUCGUGGACUACGUGGCCAGCCAGGUGACCACCGCCAAGAAGCUGCGGGGCGGCG
UGGUGUUCGUGGACGAGGUGCCCAAGGGCCUGACCGGCAAGCUGGACGCCCGGAA
GAUCCGGGAGAUCCUGAUCAAGGCCAAGAAGGGCGGCAAGAUCGCCGUGUGAGC
UGCCUUCUGCGGGGCUUGCCUUCUGGCCAUGCCCUUCUUCUCUCCCUUGCACCUG
UACCUCUUGGUCUUUGAAUAAAGCCUGAGUAGGAAGAAAAAAAAAAAAAAAAAA
AAAAAAAAAAAAAAAAAAAAAAAAAAAAAAAAAAAAAAAAAAAAAAAAAAAAAA
AAAAAAAAAAAAAAAAAAAAAAAAAAAAAAAAAAAAAAAAAAAAAAAAAAAAAA
AAAAAAAAAAAdA/3ThioMC3-MalPEG19Mal/
SEQ ID NO: 93
eGFP-S-Maleimide-PEG19-Maleimide-S-PolyA
AGGAAAUAAGAGAGAAAAGAAGAGUAAGAAGAAAUAUAAGAGCCACCAUGGUGA
GCAAGGGCGAGGAGCUGUUCACCGGGGUGGUGCCCAUCCUGGUCGAGCUGGACGG
CGACGUAAACGGCCACAAGUUCAGCGUGUCCGGCGAGGGCGAGGGCGAUGCCACC
UACGGCAAGCUGACCCUGAAGUUCAUCUGCACCACCGGCAAGCUGCCCGUGCCCU
GGCCCACCCUCGUGACCACCCUGACCUACGGCGUGCAGUGCUUCAGCCGCUACCC
CGACCACAUGAAGCAGCACGACUUCUUCAAGUCCGCCAUGCCCGAAGGCUACGUC
CAGGAGCGCACCAUCUUCUUCAAGGACGACGGCAACUACAAGACCCGCGCCGAGG
UGAAGUUCGAGGGCGACACCCUGGUGAACCGCAUCGAGCUGAAGGGCAUCGACUU
CAAGGAGGACGGCAACAUCCUGGGGCACAAGCUGGAGUACAACUACAACAGCCAC
AACGUCUAUAUCAUGGCCGACAAGCAGAAGAACGGCAUCAAGGUGAACUUCAAG
AUCCGCCACAACAUCGAGGACGGCAGCGUGCAGCUCGCCGACCACUACCAGCAGA
ACACCCCCAUCGGCGACGGCCCCGUGCUGCUGCCCGACAACCACUACCUGAGCACC
CAGUCCGCCCUGAGCAAAGACCCCAACGAGAAGCGCGAUCACAUGGUCCUGCUGG
AGUUCGUGACCGCCGCCGGGAUCACUCUCGGCAUGGACGAGCUGUACAAGUAAGC
UGCCUUCUGCGGGGCUUGCCUUCUGGCCAUGCCCUUCUUCUCUCCCUUGCACCUG
UACCUCUUGGUCUUUGAAUAAAGCCUGAGUAGGAAGAAAAAAAAAAAAAAAAAA
AAAAAAAAAAAAAAAAAAAAAAAAAAAAAAAAAAAAAAAAAAAAAAAAAAAAAA
AAAAAAAAAAAAAAAAAAAAAAAAAAAAAAAAAAAAAAAAAAAAAAAAAAAAAA
AAAAAAAAAAAdA/3ThioMC3-MalPEG19Mal-X
SEQ ID NO: 94
eGFP-S-Maleimide-PEG19-Maleimide-S-eGFP
AGGAAAUAAGAGAGAAAAGAAGAGUAAGAAGAAAUAUAAGAGCCACCAUGGUGA
GCAAGGGCGAGGAGCUGUUCACCGGGGUGGUGCCCAUCCUGGUCGAGCUGGACGG
CGACGUAAACGGCCACAAGUUCAGCGUGUCCGGCGAGGGCGAGGGCGAUGCCACC
UACGGCAAGCUGACCCUGAAGUUCAUCUGCACCACCGGCAAGCUGCCCGUGCCCU
GGCCCACCCUCGUGACCACCCUGACCUACGGCGUGCAGUGCUUCAGCCGCUACCC
CGACCACAUGAAGCAGCACGACUUCUUCAAGUCCGCCAUGCCCGAAGGCUACGUC
CAGGAGCGCACCAUCUUCUUCAAGGACGACGGCAACUACAAGACCCGCGCCGAGG
UGAAGUUCGAGGGCGACACCCUGGUGAACCGCAUCGAGCUGAAGGGCAUCGACUU
CAAGGAGGACGGCAACAUCCUGGGGCACAAGCUGGAGUACAACUACAACAGCCAC
AACGUCUAUAUCAUGGCCGACAAGCAGAAGAACGGCAUCAAGGUGAACUUCAAG
AUCCGCCACAACAUCGAGGACGGCAGCGUGCAGCUCGCCGACCACUACCAGCAGA
ACACCCCCAUCGGCGACGGCCCCGUGCUGCUGCCCGACAACCACUACCUGAGCACC
CAGUCCGCCCUGAGCAAAGACCCCAACGAGAAGCGCGAUCACAUGGUCCUGCUGG
AGUUCGUGACCGCCGCCGGGAUCACUCUCGGCAUGGACGAGCUGUACAAGUAAGC
UGCCUUCUGCGGGGCUUGCCUUCUGGCCAUGCCCUUCUUCUCUCCCUUGCACCUG
UACCUCUUGGUCUUUGAAUAAAGCCUGAGUAGGAAGAAAAAAAAAAAAAAAAAA
AAAAAAAAAAAAAAAAAAAAAAAAAAAAAAAAAAAAAAAAAAAAAAAAAAAAAA
AAAAAAAAAAAAAAAAAAAAAAAAAAAAAAAAAAAAAAAAAAAAAAAAAAAAAA
AAAAAAAAAAAdA/3ThioMC3-MalPEG19Mal-Y
Where 3ThioMC3-MalPEG19Mal-Y is

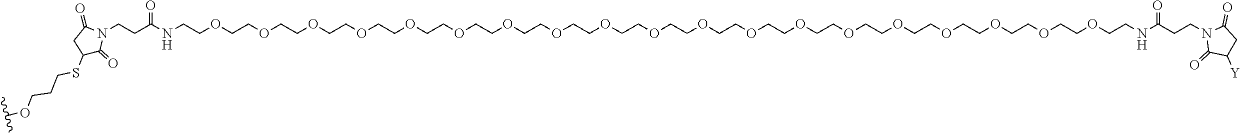

Where Y is the appropriate linkage to eGFP-SH wherein the hydrogen has been removed so as
to be connected to the maleimide with the appropriate linkage.
SEQ ID NO: 95
eGFP-SH
AGGAAAUAAGAGAGAAAAGAAGAGUAAGAAGAAAUAUAAGAGCCACCAUGGUGA
GCAAGGGCGAGGAGCUGUUCACCGGGGUGGUGCCCAUCCUGGUCGAGCUGGACGG
CGACGUAAACGGCCACAAGUUCAGCGUGUCCGGCGAGGGCGAGGGCGAUGCCACC
UACGGCAAGCUGACCCUGAAGUUCAUCUGCACCACCGGCAAGCUGCCCGUGCCCU
GGCCCACCCUCGUGACCACCCUGACCUACGGCGUGCAGUGCUUCAGCCGCUACCC
CGACCACAUGAAGCAGCACGACUUCUUCAAGUCCGCCAUGCCCGAAGGCUACGUC
CAGGAGCGCACCAUCUUCUUCAAGGACGACGGCAACUACAAGACCCGCGCCGAGG
UGAAGUUCGAGGGCGACACCCUGGUGAACCGCAUCGAGCUGAAGGGCAUCGACUU
CAAGGAGGACGGCAACAUCCUGGGGCACAAGCUGGAGUACAACUACAACAGCCAC
AACGUCUAUAUCAUGGCCGACAAGCAGAAGAACGGCAUCAAGGUGAACUUCAAG
AUCCGCCACAACAUCGAGGACGGCAGCGUGCAGCUCGCCGACCACUACCAGCAGA
ACACCCCCAUCGGCGACGGCCCCGUGCUGCUGCCCGACAACCACUACCUGAGCACC
CAGUCCGCCCUGAGCAAAGACCCCAACGAGAAGCGCGAUCACAUGGUCCUGCUGG
AGUUCGUGACCGCCGCCGGGAUCACUCUCGGCAUGGACGAGCUGUACAAGUAAGC
UGCCUUCUGCGGGGCUUGCCUUCUGGCCAUGCCCUUCUUCUCUCCCUUGCACCUG
UACCUCUUGGUCUUUGAAUAAAGCCUGAGUAGGAAGAAAAAAAAAAAAAAAAAA
AAAAAAAAAAAAAAAAAAAAAAAAAAAAAAAAAAAAAAAAAAAAAAAAAAAAAA
AAAAAAAAAAAAAAAAAAAAAAAAAAAAAAAAAAAAAAAAAAAAAAAAAAAAAA
AAAAAAAAAAAdA/3ThioMC3Reduced-D/
Where Y is the sequence for eGFP-SH with the appropriate connectivity between the 3′ thiol and
the maleimide.
SEQ ID NO: 96
eGFP-S-Maleimide-PEG19-Maleimide-S-mCherry
AGGAAAUAAGAGAGAAAAGAAGAGUAAGAAGAAAUAUAAGAGCCACCAUGGUGA
GCAAGGGCGAGGAGCUGUUCACCGGGGUGGUGCCCAUCCUGGUCGAGCUGGACGG
CGACGUAAACGGCCACAAGUUCAGCGUGUCCGGCGAGGGCGAGGGCGAUGCCACC
UACGGCAAGCUGACCCUGAAGUUCAUCUGCACCACCGGCAAGCUGCCCGUGCCCU
GGCCCACCCUCGUGACCACCCUGACCUACGGCGUGCAGUGCUUCAGCCGCUACCC
CGACCACAUGAAGCAGCACGACUUCUUCAAGUCCGCCAUGCCCGAAGGCUACGUC
CAGGAGCGCACCAUCUUCUUCAAGGACGACGGCAACUACAAGACCCGCGCCGAGG
UGAAGUUCGAGGGCGACACCCUGGUGAACCGCAUCGAGCUGAAGGGCAUCGACUU
CAAGGAGGACGGCAACAUCCUGGGGCACAAGCUGGAGUACAACUACAACAGCCAC
AACGUCUAUAUCAUGGCCGACAAGCAGAAGAACGGCAUCAAGGUGAACUUCAAG
AUCCGCCACAACAUCGAGGACGGCAGCGUGCAGCUCGCCGACCACUACCAGCAGA
ACACCCCCAUCGGCGACGGCCCCGUGCUGCUGCCCGACAACCACUACCUGAGCACC
CAGUCCGCCCUGAGCAAAGACCCCAACGAGAAGCGCGAUCACAUGGUCCUGCUGG
AGUUCGUGACCGCCGCCGGGAUCACUCUCGGCAUGGACGAGCUGUACAAGUAAGC
UGCCUUCUGCGGGGCUUGCCUUCUGGCCAUGCCCUUCUUCUCUCCCUUGCACCUG
UACCUCUUGGUCUUUGAAUAAAGCCUGAGUAGGAAGAAAAAAAAAAAAAAAAAA
AAAAAAAAAAAAAAAAAAAAAAAAAAAAAAAAAAAAAAAAAAAAAAAAAAAAAA
AAAAAAAAAAAAAAAAAAAAAAAAAAAAAAAAAAAAAAAAAAAAAAAAAAAAAA
AAAAAAAAAAAdA/3ThioMC3-MalPEG19Mal-Z
Where 3ThioMC3-MalPEG19Mal-Z is

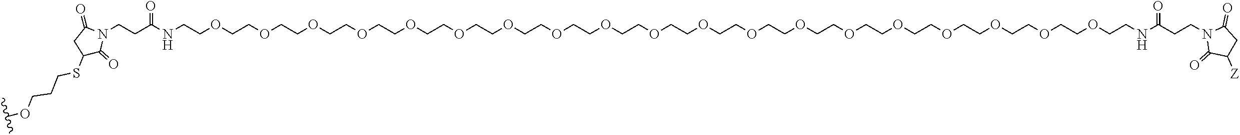

Where Z is the appropriate linkage to mCherry-SH wherein the hydrogen has been removed so
as to be connected to the maleimide with the appropriate linkage.
SEQ ID NO: 97
mCherry-SH
AGGAAAUAAGAGAGAAAAGAAGAGUAAGAAGAAAUAUAAGAGCCACCAUGGUGA
GCAAGGGCGAGGAGGACAACAUGGCCAUCAUCAAGGAGUUCAUGCGGUUCAAGG
UGCACAUGGAGGGCAGCGUGAACGGCCACGAGUUCGAGAUCGAGGGCGAGGGCG
AGGGCCGGCCCUACGAGGGCACCCAGACCGCCAAGCUGAAGGUGACCAAGGGCGG
CCCCCUGCCCUUCGCCUGGGACAUCCUGAGCCCCCAGUUCAUGUACGGCAGCAAG
GCCUACGUGAAGCACCCCGCCGACAUCCCCGACUACCUGAAGCUGAGCUUCCCCG
AGGGCUUCAAGUGGGAGCGGGUGAUGAACUUCGAGGACGGCGGCGUGGUGACCG
UGACCCAGGACAGCAGCCUGCAGGACGGCGAGUUCAUCUACAAGGUGAAGCUGCG
GGGCACCAACUUCCCCAGCGACGGCCCCGUGAUGCAGAAGAAGACCAUGGGCUGG
GAGGCCAGCAGCGAGCGGAUGUACCCCGAGGACGGCGCCCUGAAGGGCGAGAUCA
AGCAGCGGCUGAAGCUGAAGGACGGCGGCCACUACGACGCCGAGGUGAAGACCAC
CUACAAGGCCAAGAAGCCCGUGCAGCUGCCCGGCGCCUACAACGUGAACAUCAAG
CUGGACAUCACCAGCCACAACGAGGACUACACCAUCGUGGAGCAGUACGAGCGGG
CCGAGGGCCGGCACAGCACCGGCGGCAUGGACGAGCUGUACAAGAGCGGCAACUG
AGCUGCCUUCUGCGGGGCUUGCCUUCUGGCCAUGCCCUUCUUCUCUCCCUUGCAC
CUGUACCUCUUGGUCUUUGAAUAAAGCCUGAGUAGGAAGAAAAAAAAAAAAAAA
AAAAAAAAAAAAAAAAAAAAAAAAAAAAAAAAAAAAAAAAAAAAAAAAAAAAAA
AAAAAAAAAAAAAAAAAAAAAAAAAAAAAAAAAAAAAAAAAAAAAAAAAAAAAA
AAAAAAAAAAAAAAdA/3ThioMC3Reduced-D/
SEQ ID NO: 98
eGFP-S-Maleimide-SulfoCy5
AGGAAAUAAGAGAGAAAAGAAGAGUAAGAAGAAAUAUAAGAGCCACCAUGGUGA
GCAAGGGCGAGGAGCUGUUCACCGGGGUGGUGCCCAUCCUGGUCGAGCUGGACGG
CGACGUAAACGGCCACAAGUUCAGCGUGUCCGGCGAGGGCGAGGGCGAUGCCACC
UACGGCAAGCUGACCCUGAAGUUCAUCUGCACCACCGGCAAGCUGCCCGUGCCCU
GGCCCACCCUCGUGACCACCCUGACCUACGGCGUGCAGUGCUUCAGCCGCUACCC
CGACCACAUGAAGCAGCACGACUUCUUCAAGUCCGCCAUGCCCGAAGGCUACGUC
CAGGAGCGCACCAUCUUCUUCAAGGACGACGGCAACUACAAGACCCGCGCCGAGG
UGAAGUUCGAGGGCGACACCCUGGUGAACCGCAUCGAGCUGAAGGGCAUCGACUU
CAAGGAGGACGGCAACAUCCUGGGGCACAAGCUGGAGUACAACUACAACAGCCAC
AACGUCUAUAUCAUGGCCGACAAGCAGAAGAACGGCAUCAAGGUGAACUUCAAG
AUCCGCCACAACAUCGAGGACGGCAGCGUGCAGCUCGCCGACCACUACCAGCAGA
ACACCCCCAUCGGCGACGGCCCCGUGCUGCUGCCCGACAACCACUACCUGAGCACC
CAGUCCGCCCUGAGCAAAGACCCCAACGAGAAGCGCGAUCACAUGGUCCUGCUGG
AGUUCGUGACCGCCGCCGGGAUCACUCUCGGCAUGGACGAGCUGUACAAGUAAGC
UGCCUUCUGCGGGGCUUGCCUUCUGGCCAUGCCCUUCUUCUCUCCCUUGCACCUG
UACCUCUUGGUCUUUGAAUAAAGCCUGAGUAGGAAGAAAAAAAAAAAAAAAAAA
AAAAAAAAAAAAAAAAAAAAAAAAAAAAAAAAAAAAAAAAAAAAAAAAAAAAAA
AAAAAAAAAAAAAAAAAAAAAAAAAAAAAAAAAAAAAAAAAAAAAAAAAAAAAA
AAAAAAAAAAAdA/3ThioMC3-MalCy5-D/
SEQ ID NO: 99
eGFP-Cyt-S-Maleimide-PEG19-Mal
AGGAAAUAAGAGAGAAAAGAAGAGUAAGAAGAAAUAUAAGAGCCACCAUGGUGA
GCAAGGGCGAGGAGCUGUUCACCGGGGUGGUGCCCAUCCUGGUCGAGCUGGACGG
CGACGUAAACGGCCACAAGUUCAGCGUGUCCGGCGAGGGCGAGGGCGAUGCCACC
UACGGCAAGCUGACCCUGAAGUUCAUCUGCACCACCGGCAAGCUGCCCGUGCCCU
GGCCCACCCUCGUGACCACCCUGACCUACGGCGUGCAGUGCUUCAGCCGCUACCC
CGACCACAUGAAGCAGCACGACUUCUUCAAGUCCGCCAUGCCCGAAGGCUACGUC
CAGGAGCGCACCAUCUUCUUCAAGGACGACGGCAACUACAAGACCCGCGCCGAGG
UGAAGUUCGAGGGCGACACCCUGGUGAACCGCAUCGAGCUGAAGGGCAUCGACUU
CAAGGAGGACGGCAACAUCCUGGGGCACAAGCUGGAGUACAACUACAACAGCCAC
AACGUCUAUAUCAUGGCCGACAAGCAGAAGAACGGCAUCAAGGUGAACUUCAAG
AUCCGCCACAACAUCGAGGACGGCAGCGUGCAGCUCGCCGACCACUACCAGCAGA
ACACCCCCAUCGGCGACGGCCCCGUGCUGCUGCCCGACAACCACUACCUGAGCACC
CAGUCCGCCCUGAGCAAAGACCCCAACGAGAAGCGCGAUCACAUGGUCCUGCUGG
AGUUCGUGACCGCCGCCGGGAUCACUCUCGGCAUGGACGAGCUGUACAAGUAAGC
UGCCUUCUGCGGGGCUUGCCUUCUGGCCAUGCCCUUCUUCUCUCCCUUGCACCUG
UACCUCUUGGUCUUUGAAUAAAGCCUGAGUAGGAAGAAAAAAAAAAAAAAAAAA
AAAAAAAAAAAAAAAAAAAAAAAAAAAAAAAAAAAAAAAAAAAAAAAAAAAAAA
AAAAAAAAAAAAAAAAAAAAAAAAAAAAAAAAAAAAAAAAAAAAAAAAA/Cp-
Maleimide-PEG19-Mal
Where Cp-Maleimide-PEG19-Maleimide is the following structure

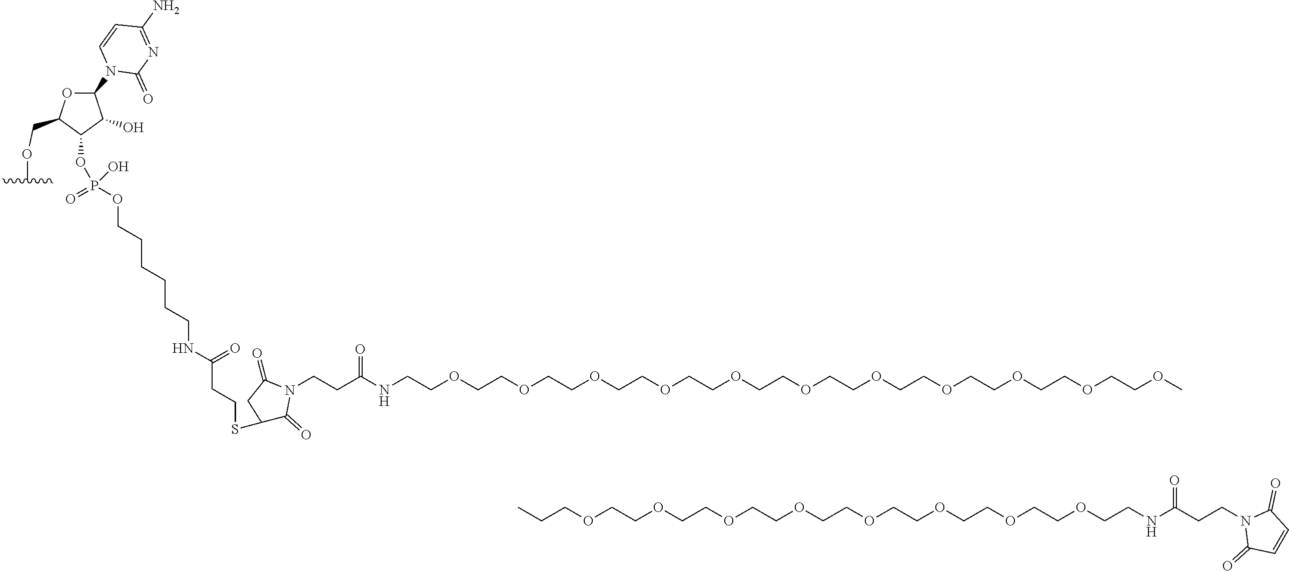

SEQ ID NO: 100
eGFP-S-Maleimide-PEG19-Maleimide-Peptide
AGGAAAUAAGAGAGAAAAGAAGAGUAAGAAGAAAUAUAAGAGCCACCAUGGUGA
GCAAGGGCGAGGAGCUGUUCACCGGGGUGGUGCCCAUCCUGGUCGAGCUGGACGG
CGACGUAAACGGCCACAAGUUCAGCGUGUCCGGCGAGGGCGAGGGCGAUGCCACC
UACGGCAAGCUGACCCUGAAGUUCAUCUGCACCACCGGCAAGCUGCCCGUGCCCU
GGCCCACCCUCGUGACCACCCUGACCUACGGCGUGCAGUGCUUCAGCCGCUACCC
CGACCACAUGAAGCAGCACGACUUCUUCAAGUCCGCCAUGCCCGAAGGCUACGUC
CAGGAGCGCACCAUCUUCUUCAAGGACGACGGCAACUACAAGACCCGCGCCGAGG
UGAAGUUCGAGGGCGACACCCUGGUGAACCGCAUCGAGCUGAAGGGCAUCGACUU
CAAGGAGGACGGCAACAUCCUGGGGCACAAGCUGGAGUACAACUACAACAGCCAC
AACGUCUAUAUCAUGGCCGACAAGCAGAAGAACGGCAUCAAGGUGAACUUCAAG
AUCCGCCACAACAUCGAGGACGGCAGCGUGCAGCUCGCCGACCACUACCAGCAGA
ACACCCCCAUCGGCGACGGCCCCGUGCUGCUGCCCGACAACCACUACCUGAGCACC
CAGUCCGCCCUGAGCAAAGACCCCAACGAGAAGCGCGAUCACAUGGUCCUGCUGG
AGUUCGUGACCGCCGCCGGGAUCACUCUCGGCAUGGACGAGCUGUACAAGUAAGC
UGCCUUCUGCGGGGCUUGCCUUCUGGCCAUGCCCUUCUUCUCUCCCUUGCACCUG
UACCUCUUGGUCUUUGAAUAAAGCCUGAGUAGGAAGAAAAAAAAAAAAAAAAAA
AAAAAAAAAAAAAAAAAAAAAAAAAAAAAAAAAAAAAAAAAAAAAAAAAAAAAA
AAAAAAAAAAAAAAAAAAAAAAAAAAAAAAAAAAAAAAAAAAAAAAAAA/Cp-
Maleimide-PEG19-Maleimide-peptide
Where Cp-Maleimide-PEG19-Maleimide-Peptide is the following structure

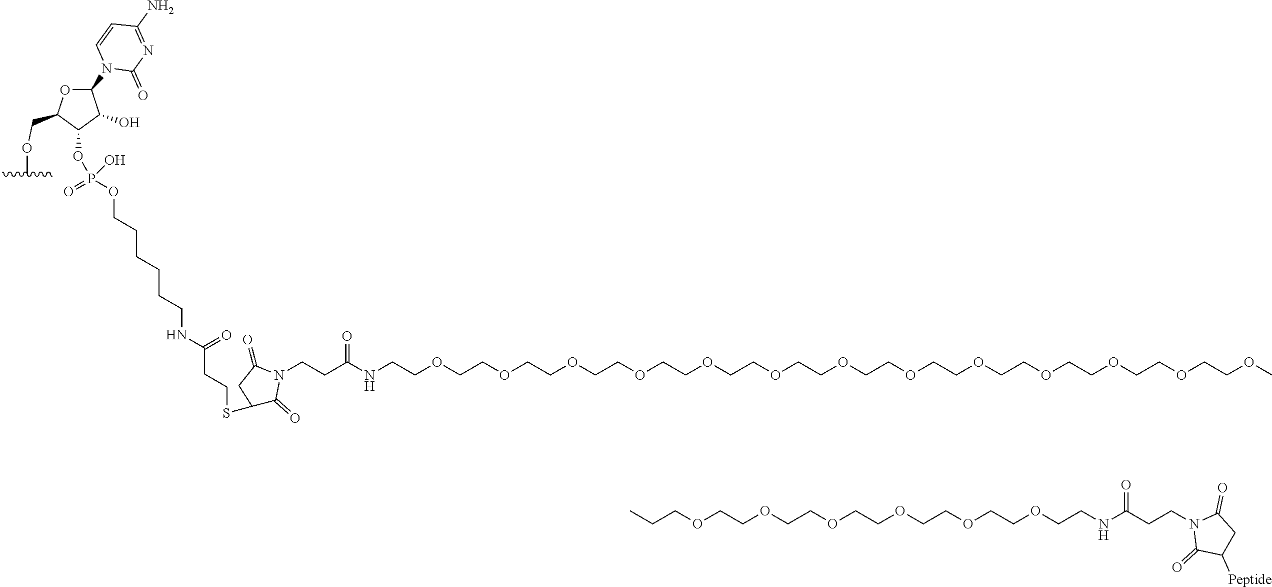

SEQ ID NO: 101
Peptide 1
PASPASPASPASPASC-NH₂
SEQ ID NO: 102
Peptide 2
PASPASPASPASPASPASPASPASPASPASPASPASPASPASPASC-NH₂
SEQ ID NO: 103
Peptide 3
CPASPASPASPASPASKDEL-NH₂
SEQ ID NO: 104
Peptide 4
CPASPASPASPASPASDEKKMP-NH₂
SEQ ID NO: 105
Peptide 5
RGVPHIVMVDAYKRYKSGGSC-NH₂
SEQ ID NO: 106
Peptide 6
VTTLSGLSGEQGPSGDMTTEEDSATHIKFSKRDEDGRELAGATMELRDCSGKTISTWISD
GHVKDFYLYPGKYTFVETAAPDGYEVATPIEFTVNEDGQVTVDGEATEGDAHT-NH₂
SEQ ID NO: 107
Peptide 7
Y-Aib-EGTFTSDYSI-Aib-LDKIAQK(GGC)AFVQWLIAGGPSSGAPPPS-NH₂
SEQ ID NO: 108
Adalimumab HC Nucleic Acid
AGGAAAUAAGAGAGAAAAGAAGAGUAAGAAGAAAUAUAAGAGCCACCAUGGAAG
UGCAGCUGGUGGAAAGCGGCGGCGGCCUGGUGCAGCCGGGCCGCAGCCUGCGCCU
GAGCUGCGCGGCGAGCGGCUUUACCUUUGAUGAUUAUGCGAUGCAUUGGGUGCG
CCAGGCGCCGGGCAAAGGCCUGGAAUGGGUGAGCGCGAUUACCUGGAACAGCGGC
CAUAUUGAUUAUGCGGAUAGCGUGGAAGGCCGCUUUACCAUUAGCCGCGAUAAC
GCGAAAAACAGCCUGUAUCUGCAGAUGAACAGCCUGCGCGCGGAAGAUACCGCGG
UGUAUUAUUGCGCGAAAGUGAGCUAUCUGAGCACCGCGAGCAGCCUGGAUUAUU
GGGGCCAGGGCACCCUGGUGACCGUGAGCAGCGCGAGCACCAAAGGCCCGAGCGU
GUUUCCGCUGGCGCCGAGCAGCAAAAGCACCAGCGGCGGCACCGCGGCGCUGGGC
UGCCUGGUGAAAGAUUAUUUUCCGGAACCGGUGACCGUGAGCUGGAACAGCGGC
GCGCUGACCAGCGGCGUGCAUACCUUUCCGGCGGUGCUGCAGAGCAGCGGCCUGU
AUAGCCUGAGCAGCGUGGUGACCGUGCCGAGCAGCAGCCUGGGCACCCAGACCUA
UAUUUGCAACGUGAACCAUAAACCGAGCAACACCAAAGUGGAUAAAAAAGUGGA
ACCGAAAAGCUGCGAUAAAACCCAUACCUGCCCGCCGUGCCCGGCGCCGGAACUG
CUGGGCGGCCCGAGCGUGUUUCUGUUUCCGCCGAAACCGAAAGAUACCCUGAUGA
UUAGCCGCACCCCGGAAGUGACCUGCGUGGUGGUGGAUGUGAGCCAUGAAGAUCC
GGAAGUGAAAUUUAACUGGUAUGUGGAUGGCGUGGAAGUGCAUAACGCGAAAAC
CAAACCGCGCGAAGAACAGUAUAACAGCACCUAUCGCGUGGUGAGCGUGCUGACC
GUGCUGCAUCAGGAUUGGCUGAACGGCAAAGAAUAUAAAUGCAAAGUGAGCAAC
AAAGCGCUGCCGGCGCCGAUUGAAAAAACCAUUAGCAAAGCGAAAGGCCAGCCGC
GCGAACCGCAGGUGUAUACCCUGCCGCCGAGCCGCGAUGAACUGACCAAAAACCA
GGUGAGCCUGACCUGCCUGGUGAAAGGCUUUUAUCCGAGCGAUAUUGCGGUGGA
AUGGGAAAGCAACGGCCAGCCGGAAAACAACUAUAAAACCACCCCGCCGGUGCUG
GAUAGCGAUGGCAGCUUUUUUCUGUAUAGCAAACUGACCGUGGAUAAAAGCCGC
UGGCAGCAGGGCAACGUGUUUAGCUGCAGCGUGAUGCAUGAAGCGCUGCAUAAC
CAUUAUACCCAGAAAAGCCUGAGCCUGAGCCCGGGCAAAGCUGCCUUCUGCGGGG
CUUGCCUUCUGGCCAUGCCCUUCUUCUCUCCCUUGCACCUGUACCUCUUGGUCUU
UGAAUAAAGCCUGAGUAGGAAGAAAAAAAAAAAAAAAAAAAAAAAAAAAAAAAA
AAAAAAAAAAAAAAAAAAAAAAAAAAAAAAAAAAAAAAAAAAAAAAAA
SEQ ID NO: 109
Adalimumab HC Amino Acid
EVQLVESGGGLVQPGRSLRLSCAASGFTFDDYAMHWVRQAPGKGLEWVSAITWNSGHI
DYADSVEGRFTISRDNAKNSLYLQMNSLRAEDTAVYYCAKVSYLSTASSLDYWGQGTL
VTVSSASTKGPSVFPLAPSSKSTSGGTAALGCLVKDYFPEPVTVSWNSGALTSGVHTFPA
VLQSSGLYSLSSVVTVPSSSLGTQTYICNVNHKPSNTKVDKKVEPKSCDKTHTCPPCPAP
ELLGGPSVFLFPPKPKDTLMISRTPEVTCVVVDVSHEDPEVKFNWYVDGVEVHNAKTKP
REEQYNSTYRVVSVLTVLHQDWLNGKEYKCKVSNKALPAPIEKTISKAKGQPREPQVY
TLPPSRDELTKNQVSLTCLVKGFYPSDIAVEWESNGQPENNYKTTPPVLDSDGSFFLYSK
LTVDKSRWQQGNVFSCSVMHEALHNHYTQKSLSLSPGK
SEQ ID NO: 110
Adalimumab LC Nucleic Acid
AGGAAAUAAGAGAGAAAAGAAGAGUAAGAAGAAAUAUAAGAGCCACCAUGGAUA
UUCAGAUGACCCAGAGCCCGAGCAGCCUGAGCGCGAGCGUGGGCGAUCGCGUGAC
CAUUACCUGCCGCGCGAGCCAGGGCAUUCGCAACUAUCUGGCGUGGUAUCAGCAG
AAACCGGGCAAAGCGCCGAAACUGCUGAUUUAUGCGGCGAGCACCCUGCAGAGCG
GCGUGCCGAGCCGCUUUAGCGGCAGCGGCAGCGGCACCGAUUUUACCCUGACCAU
UAGCAGCCUGCAGCCGGAAGAUGUGGCGACCUAUUAUUGCCAGCGCUAUAACCGC
GCGCCGUAUACCUUUGGCCAGGGCACCAAAGUGGAAAUUAAACGCACCGUGGCGG
CGCCGAGCGUGUUUAUUUUUCCGCCGAGCGAUGAACAGCUGAAAAGCGGCACCGC
GAGCGUGGUGUGCCUGCUGAACAACUUUUAUCCGCGCGAAGCGAAAGUGCAGUG
GAAAGUGGAUAACGCGCUGCAGAGCGGCAACAGCCAGGAAAGCGUGACCGAACA
GGAUAGCAAAGAUAGCACCUAUAGCCUGAGCAGCACCCUGACCCUGAGCAAAGCG
GAUUAUGAAAAACAUAAAGUGUAUGCGUGCGAAGUGACCCAUCAGGGCCUGAGC
AGCCCGGUGACCAAAAGCUUUAACCGCGGCGAAUGCGCUGCCUUCUGCGGGGCUU
GCCUUCUGGCCAUGCCCUUCUUCUCUCCCUUGCACCUGUACCUCUUGGUCUUUGA
AUAAAGCCUGAGUAGGAAGAAAAAAAAAAAAAAAAAAAAAAAAAAAAAAAAAAA
AAAAAAAAAAAAAAAAAAAAAAAAAAAAAAAAAAAAAAAAAAAAA
SEQ ID NO: 111
Adalimumab LC Amino Acid
DIQMTQSPSSLSASVGDRVTITCRASQGIRNYLAWYQQKPGKAPKLLIYAASTLQSGVPS
RFSGSGSGTDFTLTISSLQPEDVATYYCQRYNRAPYTFGQGTKVEIKRTVAAPSVFIFPPS
DEQLKSGTASVVCLLNNFYPREAKVQWKVDNALQSGNSQESVTEQDSKDSTYSLSSTL
TLSKADYEKHKVYACEVTHQGLSSPVTKSFNRGEC
SEQ ID NO: 112
Duplimab HC Nucleic Acid
AGGAAAUAAGAGAGAAAAGAAGAGUAAGAAGAAAUAUAAGAGCCACCAUGGAAG
UGCAGCUGGUGGAAAGCGGCGGCGGCCUGGAACAGCCGGGCGGCAGCCUGCGCCU
GAGCUGCGCGGGCAGCGGCUUUACCUUUCGCGAUUAUGCGAUGACCUGGGUGCGC
CAGGCGCCGGGCAAAGGCCUGGAAUGGGUGAGCAGCAUUAGCGGCAGCGGCGGCA
ACACCUAUUAUGCGGAUAGCGUGAAAGGCCGCUUUACCAUUAGCCGCGAUAACAG
CAAAAACACCCUGUAUCUGCAGAUGAACAGCCUGCGCGCGGAAGAUACCGCGGUG
UAUUAUUGCGCGAAAGAUCGCCUGAGCAUUACCAUUCGCCCGCGCUAUUAUGGCC
UGGAUGUGUGGGGCCAGGGCACCACCGUGACCGUGAGCAGCGCGAGCACCAAAGG
CCCGAGCGUGUUUCCGCUGGCGCCGUGCAGCCGCAGCACCAGCGAAAGCACCGCG
GCGCUGGGCUGCCUGGUGAAAGAUUAUUUUCCGGAACCGGUGACCGUGAGCUGG
AACAGCGGCGCGCUGACCAGCGGCGUGCAUACCUUUCCGGCGGUGCUGCAGAGCA
GCGGCCUGUAUAGCCUGAGCAGCGUGGUGACCGUGCCGAGCAGCAGCCUGGGCAC
CAAAACCUAUACCUGCAACGUGGAUCAUAAACCGAGCAACACCAAAGUGGAUAAA
CGCGUGGAAAGCAAAUAUGGCCCGCCGUGCCCGCCGUGCCCGGCGCCGGAAUUUC
UGGGCGGCCCGAGCGUGUUUCUGUUUCCGCCGAAACCGAAAGAUACCCUGAUGAU
UAGCCGCACCCCGGAAGUGACCUGCGUGGUGGUGGAUGUGAGCCAGGAAGAUCCG
GAAGUGCAGUUUAACUGGUAUGUGGAUGGCGUGGAAGUGCAUAACGCGAAAACC
AAACCGCGCGAAGAACAGUUUAACAGCACCUAUCGCGUGGUGAGCGUGCUGACCG
UGCUGCAUCAGGAUUGGCUGAACGGCAAAGAAUAUAAAUGCAAAGUGAGCAACA
AAGGCCUGCCGAGCAGCAUUGAAAAAACCAUUAGCAAAGCGAAAGGCCAGCCGCG
CGAACCGCAGGUGUAUACCCUGCCGCCGAGCCAGGAAGAAAUGACCAAAAACCAG
GUGAGCCUGACCUGCCUGGUGAAAGGCUUUUAUCCGAGCGAUAUUGCGGUGGAA
UGGGAAAGCAACGGCCAGCCGGAAAACAACUAUAAAACCACCCCGCCGGUGCUGG
AUAGCGAUGGCAGCUUUUUUCUGUAUAGCCGCCUGACCGUGGAUAAAAGCCGCU
GGCAGGAAGGCAACGUGUUUAGCUGCAGCGUGAUGCAUGAAGCGCUGCAUAACC
AUUAUACCCAGAAAAGCCUGAGCCUGAGCCUGGGCGCUGCCUUCUGCGGGGCUUG
CCUUCUGGCCAUGCCCUUCUUCUCUCCCUUGCACCUGUACCUCUUGGUCUUUGAA
UAAAGCCUGAGUAGGAAGAAAAAAAAAAAAAAAAAAAAAAAAAAAAAAAAAAAA
AAAAAAAAAAAAAAAAAAAAAAAAAAAAAAAAAAAAAAAAAAAA
SEQ ID NO: 113
Duplimab HC Amino Acid
EVQLVESGGGLEQPGGSLRLSCAGSGFTFRDYAMTWVRQAPGKGLEWVSSISGSGGNT
YYADSVKGRFTISRDNSKNTLYLQMNSLRAEDTAVYYCAKDRLSITIRPRYYGLDVWG
QGTTVTVSSASTKGPSVFPLAPCSRSTSESTAALGCLVKDYFPEPVTVSWNSGALTSGVH
TFPAVLQSSGLYSLSSVVTVPSSSLGTKTYTCNVDHKPSNTKVDKRVESKYGPPCPPCPA
PEFLGGPSVFLFPPKPKDTLMISRTPEVTCVVVDVSQEDPEVQFNWYVDGVEVHNAKTK
PREEQFNSTYRVVSVLTVLHQDWLNGKEYKCKVSNKGLPSSIEKTISKAKGQPREPQVY
TLPPSQEEMTKNQVSLTCLVKGFYPSDIAVEWESNGQPENNYKTTPPVLDSDGSFFLYSR
LTVDKSRWQEGNVFSCSVMHEALHNHYTQKSLSLSLG
SEQ ID NO: 114
Duplimab LC Nucleic Acid
AGGAAAUAAGAGAGAAAAGAAGAGUAAGAAGAAAUAUAAGAGCCACCAUGGAUA
UUGUGAUGACCCAGAGCCCGCUGAGCCUGCCGGUGACCCCGGGCGAACCGGCGAG
CAUUAGCUGCCGCAGCAGCCAGAGCCUGCUGUAUAGCAUUGGCUAUAACUAUCUG
GAUUGGUAUCUGCAGAAAAGCGGCCAGAGCCCGCAGCUGCUGAUUUAUCUGGGC
AGCAACCGCGCGAGCGGCGUGCCGGAUCGCUUUAGCGGCAGCGGCAGCGGCACCG
AUUUUACCCUGAAAAUUAGCCGCGUGGAAGCGGAAGAUGUGGGCUUUUAUUAUU
GCAUGCAGGCGCUGCAGACCCCGUAUACCUUUGGCCAGGGCACCAAACUGGAAAU
UAAACGCACCGUGGCGGCGCCGAGCGUGUUUAUUUUUCCGCCGAGCGAUGAACAG
CUGAAAAGCGGCACCGCGAGCGUGGUGUGCCUGCUGAACAACUUUUAUCCGCGCG
AAGCGAAAGUGCAGUGGAAAGUGGAUAACGCGCUGCAGAGCGGCAACAGCCAGG
AAAGCGUGACCGAACAGGAUAGCAAAGAUAGCACCUAUAGCCUGAGCAGCACCCU
GACCCUGAGCAAAGCGGAUUAUGAAAAACAUAAAGUGUAUGCGUGCGAAGUGAC
CCAUCAGGGCCUGAGCAGCCCGGUGACCAAAAGCUUUAACCGCGGCGAAUGCGCU
GCCUUCUGCGGGGCUUGCCUUCUGGCCAUGCCCUUCUUCUCUCCCUUGCACCUGU
ACCUCUUGGUCUUUGAAUAAAGCCUGAGUAGGAAGAAAAAAAAAAAAAAAAAAA
AAAAAAAAAAAAAAAAAAAAAAAAAAAAAAAAAAAAAAAAAAAAAAAAAAAAAA
AAAAAAA
SEQ ID NO: 115
Duplimab LC Amino Acid
DIVMTQSPLSLPVTPGEPASISCRSSQSLLYSIGYNYLDWYLQKSGQSPQLLIYLGSNRAS
GVPDRFSGSGSGTDFTLKISRVEAEDVGFYYCMQALQTPYTFGQGTKLEIKRTVAAPSV
FIFPPSDEQLKSGTASVVCLLNNFYPREAKVQWKVDNALQSGNSQESVTEQDSKDSTYS
LSSTLTLSKADYEKHKVYACEVTHQGLSSPVTKSFNRGEC
SEQ ID NO: 116
Trastuzumab HC Nucleic Acid
AGGAAAUAAGAGAGAAAAGAAGAGUAAGAAGAAAUAUAAGAGCCACCAUGGAAG
UGCAGCUGGUGGAAAGCGGCGGCGGCCUGGUGCAGCCGGGCGGCAGCCUGCGCCU
GAGCUGCGCGGCGAGCGGCUUUAACAUUAAAGAUACCUAUAUUCAUUGGGUGCG
CCAGGCGCCGGGCAAAGGCCUGGAAUGGGUGGCGCGCAUUUAUCCGACCAACGGC
UAUACCCGCUAUGCGGAUAGCGUGAAAGGCCGCUUUACCAUUAGCGCGGAUACCA
GCAAAAACACCGCGUAUCUGCAGAUGAACAGCCUGCGCGCGGAAGAUACCGCGGU
GUAUUAUUGCAGCCGCUGGGGCGGCGAUGGCUUUUAUGCGAUGGAUUAUUGGGG
CCAGGGCACCCUGGUGACCGUGAGCAGCGCGAGCACCAAAGGCCCGAGCGUGUUU
CCGCUGGCGCCGAGCAGCAAAAGCACCAGCGGCGGCACCGCGGCGCUGGGCUGCC
UGGUGAAAGAUUAUUUUCCGGAACCGGUGACCGUGAGCUGGAACAGCGGCGCGC
UGACCAGCGGCGUGCAUACCUUUCCGGCGGUGCUGCAGAGCAGCGGCCUGUAUAG
CCUGAGCAGCGUGGUGACCGUGCCGAGCAGCAGCCUGGGCACCCAGACCUAUAUU
UGCAACGUGAACCAUAAACCGAGCAACACCAAAGUGGAUAAAAAAGUGGAACCG
AAAAGCUGCGAUAAAACCCAUACCUGCCCGCCGUGCCCGGCGCCGGAACUGCUGG
GCGGCCCGAGCGUGUUUCUGUUUCCGCCGAAACCGAAAGAUACCCUGAUGAUUAG
CCGCACCCCGGAAGUGACCUGCGUGGUGGUGGAUGUGAGCCAUGAAGAUCCGGAA
GUGAAAUUUAACUGGUAUGUGGAUGGCGUGGAAGUGCAUAACGCGAAAACCAAA
CCGCGCGAAGAACAGUAUAACAGCACCUAUCGCGUGGUGAGCGUGCUGACCGUGC
UGCAUCAGGAUUGGCUGAACGGCAAAGAAUAUAAAUGCAAAGUGAGCAACAAAG
CGCUGCCGGCGCCGAUUGAAAAAACCAUUAGCAAAGCGAAAGGCCAGCCGCGCGA
ACCGCAGGUGUAUACCCUGCCGCCGAGCCGCGAAGAAAUGACCAAAAACCAGGUG
AGCCUGACCUGCCUGGUGAAAGGCUUUUAUCCGAGCGAUAUUGCGGUGGAAUGG
GAAAGCAACGGCCAGCCGGAAAACAACUAUAAAACCACCCCGCCGGUGCUGGAUA
GCGAUGGCAGCUUUUUUCUGUAUAGCAAACUGACCGUGGAUAAAAGCCGCUGGC
AGCAGGGCAACGUGUUUAGCUGCAGCGUGAUGCAUGAAGCGCUGCAUAACCAUU
AUACCCAGAAAAGCCUGAGCCUGAGCCCGGGCGCUGCCUUCUGCGGGGCUUGCCU
UCUGGCCAUGCCCUUCUUCUCUCCCUUGCACCUGUACCUCUUGGUCUUUGAAUAA
AGCCUGAGUAGGAAGAAAAAAAAAAAAAAAAAAAAAAAAAAAAAAAAAAAAAAA
AAAAAAAAAAAAAAAAAAAAAAAAAAAAAAAAAAAAAAAAA
SEQ ID NO: 117
Trastuzumab HC Amino Acid
EVQLVESGGGLVQPGGSLRLSCAASGFNIKDTYIHWVRQAPGKGLEWVARIYPTNGYT
RYADSVKGRFTISADTSKNTAYLQMNSLRAEDTAVYYCSRWGGDGFYAMDYWQGTL
VTVSSASTKGPSVFPLAPSSKSTSGGTAALGCLVKDYFPEPVTVSWNSGALTSGVHTFPA
VLQSSGLYSLSSVVTVPSSSLGTQTYICNVNHKPSNTKVDKKVEPKSCDKTHTCPPCPAP
ELLGGPSVFLFPPKPKDTLMISRTPEVTCVVVDVSHEDPEVKFNWYVDGVEVHNAKTKP
REEQYNSTYRVVSVLTVLHQDWLNGKEYKCKVSNKALPAPIEKTISKAKGQPREPQVY
TLPPSREEMTKNQVSLTCLVKGFYPSDIAVEWESNGQPENNYKTTPPVLDSDGSFFLYSK
LTVDKSRWQQGNVFSCSVMHEALHNHYTQKSLSLSPG
SEQ ID NO: 118
Trastuzumab LC Nucleic Acid
AGGAAAUAAGAGAGAAAAGAAGAGUAAGAAGAAAUAUAAGAGCCACCAUGGAUA
UUCAGAUGACCCAGAGCCCGAGCAGCCUGAGCGCGAGCGUGGGCGAUCGCGUGAC
CAUUACCUGCCGCGCGAGCCAGGAUGUGAACACCGCGGUGGCGUGGUAUCAGCAG
AAACCGGGCAAAGCGCCGAAACUGCUGAUUUAUAGCGCGAGCUUUCUGUAUAGC
GGCGUGCCGAGCCGCUUUAGCGGCAGCCGCAGCGGCACCGAUUUUACCCUGACCA
UUAGCAGCCUGCAGCCGGAAGAUUUUGCGACCUAUUAUUGCCAGCAGCAUUAUAC
CACCCCGCCGACCUUUGGCCAGGGCACCAAAGUGGAAAUUAAACGCACCGUGGCG
GCGCCGAGCGUGUUUAUUUUUCCGCCGAGCGAUGAACAGCUGAAAAGCGGCACCG
CGAGCGUGGUGUGCCUGCUGAACAACUUUUAUCCGCGCGAAGCGAAAGUGCAGU
GGAAAGUGGAUAACGCGCUGCAGAGCGGCAACAGCCAGGAAAGCGUGACCGAAC
AGGAUAGCAAAGAUAGCACCUAUAGCCUGAGCAGCACCCUGACCCUGAGCAAAGC
GGAUUAUGAAAAACAUAAAGUGUAUGCGUGCGAAGUGACCCAUCAGGGCCUGAG
CAGCCCGGUGACCAAAAGCUUUAACCGCGGCGAAUGCGCUGCCUUCUGCGGGGCU
UGCCUUCUGGCCAUGCCCUUCUUCUCUCCCUUGCACCUGUACCUCUUGGUCUUUG
AAUAAAGCCUGAGUAGGAAGAAAAAAAAAAAAAAAAAAAAAAAAAAAAAAAAAA
AAAAAAAAAAAAAAAAAAAAAAAAAAAAAAAAAAAAAAAAAAAAAA
SEQ ID NO: 119
Trastuzumab LC Amino Acid
DIQMTQSPSSLSASVGDRVTITCRASQDVNAVAWYQQKPGKAPKLLIYSASFLYSGVPS
RFSGSRSGTDFTLTISSLQPEDFATYYCQQHYTTPPTFGQGTKVEIKRTVAAPSVFIFPPSD
EQLKSGTASVVCLLNNFYPREAKVQWKVDNALQSGNSQESVTEQDSKDSTYSLSSTLTL
SKADYEKHKVYACEVTHQGLSSPVTKSFNRGEC
SEQ ID NO: 120
Exenatide Nucleic Acid
AGGAAAUAAGAGAGAAAAGAAGAGUAAGAAGAAAUAUAAGAGCCACCAUGCAUG
GCGAAGGCACCUUUACCAGCGAUCUGAGCAAACAGAUGGAAGAAGAAGCGGUGC
GCCUGUUUAUUGAAUGGCUGAAAAACGGCGGCCCGAGCAGCGGCGCGCCGCCGCC
GAGCGCUGCCUUCUGCGGGGCUUGCCUUCUGGCCAUGCCCUUCUUCUCUCCCUUG
CACCUGUACCUCUUGGUCUUUGAAUAAAGCCUGAGUAGGAAGAAAAAAAAAAAA
AAAAAAAAAAAAAAAAAAAAAAAAAAAAAAAAAAAAAAAAAAAAAAAAAAAAAA
AAAAAAAAAAAAAA
SEQ ID NO: 121
Exenatide Amino Acid
HGEGTFTSDLSKQMEEEAVRLFIEWLKNGGPSSGAPPPS
SEQ ID NO: 122
GLP-1 Nucleic Acid
AGGAAAUAAGAGAGAAAAGAAGAGUAAGAAGAAAUAUAAGAGCCACCAUGCAUG
CGGAAGGCACCUUUACCAGCGAUGUGAGCAGCUAUCUGGAAGGCCAGGCGGCGAA
AGAAUUUAUUGCGUGGCUGGUGAAAGGCCGCGGCGCUGCCUUCUGCGGGGCUUG
CCUUCUGGCCAUGCCCUUCUUCUCUCCCUUGCACCUGUACCUCUUGGUCUUUGAA
UAAAGCCUGAGUAGGAAGAAAAAAAAAAAAAAAAAAAAAAAAAAAAAAAAAAAA
AAAAAAAAAAAAAAAAAAAAAAAAAAAAAAAAAAAAAAAAAAAA
SEQ ID NO: 123
GLP-1 Amino Acid
HAEGTFTSDVSSYLEGQAAKEFIAWLVKGRG
SEQ ID NO: 124
Gastric Inhibitory Peptide Nucleic Acid
AGGAAAUAAGAGAGAAAAGAAGAGUAAGAAGAAAUAUAAGAGCCACCAUGUAUG
CGGAAGGCACCUUUAUUAGCGAUUAUAGCAUUGCGAUGGAUAAAAUUCAUCAGC
AGGAUUUUGUGAACUGGCUGCUGGCGCAGAAAGGCAAAAAAAACGAUUGGAAAC
AUAACAUUACCCAGGCUGCCUUCUGCGGGGCUUGCCUUCUGGCCAUGCCCUUCUU
CUCUCCCUUGCACCUGUACCUCUUGGUCUUUGAAUAAAGCCUGAGUAGGAAGAAA
AAAAAAAAAAAAAAAAAAAAAAAAAAAAAAAAAAAAAAAAAAAAAAAAAAAAAA
AAAAAAAAAAAAAAAAAAAAAAA
SEQ ID NO: 125
Gastric Inhibitory Peptide Amino Acid
YAEGTFISDYSIAMDKIHQQDFVNWLLAQKGKKNDWKHNITQ
SEQ ID NO: 126
Tariparatide Nucleic Acid
AGGAAAUAAGAGAGAAAAGAAGAGUAAGAAGAAAUAUAAGAGCCACCAUGAGCG
UGAGCGAAAUUCAGCUGAUGCAUAACCUGGGCAAACAUCUGAACAGCAUGGAAC
GCGUGGAAUGGCUGCGCAAAAAACUGCAGGAUGUGCAUAACUUUGCUGCCUUCU
GCGGGGCUUGCCUUCUGGCCAUGCCCUUCUUCUCUCCCUUGCACCUGUACCUCUU
GGUCUUUGAAUAAAGCCUGAGUAGGAAGAAAAAAAAAAAAAAAAAAAAAAAAAA
AAAAAAAAAAAAAAAAAAAAAAAAAAAAAAAAAAAAAAAAAAAAAAAAAAAAAA
SEQ ID NO: 127
Tariparatide Amino Acid
SVSEIQLMHNLGKHLNSMERVEWLRKKLQDVHNF
SEQ ID NO: 128
Pembrolizumab Heavy Chain (PembrolizumabHC)
AGGAAAUAAGAGAGAAAAGAAGAGUAAGAAGAAAUAUAAGAGCCACCAUGAGGG
CUUGGAUCUUCUUUCUGCUCUGCCUGGCCGGGCGCGCCUUGGCCCAAGUGCAGCU
GGUGCAGAGCGGUGUAGAGGUCAAGAAACCCGGCGCUAGCGUGAAGGUGAGCUG
CAAGGCUAGCGGCUACACCUUCACCAACUACUACAUGUACUGGGUGAGACAAGCC
CCCGGCCAAGGCCUGGAGUGGAUGGGCGGCAUCAACCCUAGCAACGGCGGCACCA
ACUUCAACGAGAAGUUCAAGAACAGAGUGACCCUGACCACCGACAGCAGCACCAC
CACCGCCUACAUGGAGCUGAAAAGCCUGCAGUUCGACGACACCGCCGUGUACUAC
UGCGCUAGAAGAGACUACAGAUUCGACAUGGGCUUCGACUACUGGGGCCAAGGC
ACCACCGUGACCGUGAGCAGCGCUAGCACCAAGGGUCCUAGCGUUUUCCCCCUAG
CCCCCUGCAGCAGAAGCACAAGCGAGAGCACCGCCGCCCUGGGCUGCCUUGUCAA
GGACUAUUUCCCCGAGCCCGUGACCGUAUCCUGGAACAGCGGCGCACUUACGUCC
GGCGUGCACACCUUCCCCGCCGUGCUGCAGAGCAGCGGCCUGUACAGCCUGAGCA
GCGUGGUGACCGUGCCUUCGAGCAGCCUGGGCACCAAGACCUACACCUGCAACGU
GGACCACAAGCCUAGCAACACCAAGGUGGACAAGAGAGUUGAGUCCAAGUAUGG
ACCCCCGUGUCCACCCUGUCCCGCCCCGGAGUUCCUGGGCGGCCCUAGCGUAUUC
CUAUUUCCCCCCAAGCCCAAGGACACCCUGAUGAUCAGCAGAACCCCCGAGGUGA
CCUGCGUGGUGGUGGACGUGAGCCAAGAGGACCCCGAGGUGCAGUUCAACUGGU
ACGUGGACGGGGUGGAAGUUCACAACGCCAAGACCAAGCCUAGAGAGGAGCAGU
UCAACAGCACCUACAGAGUGGUGAGCGUGCUGACCGUGCUGCACCAAGACUGGCU
GAACGGCAAGGAGUACAAGUGCAAGGUGAGCAACAAGGGCCUGCCUAGCAGCAU
CGAGAAGACCAUCAGCAAGGCCAAGGGGCAGCCUAGAGAGCCCCAAGUGUACACC
CUGCCCCCUAGCCAAGAGGAGAUGACCAAGAACCAAGUGAGCCUGACCUGCCUUG
UAAAGGGCUUCUACCCUAGCGACAUCGCCGUGGAGUGGGAGAGCAACGGGCAGCC
CGAGAACAACUACAAGACCACCCCCCCCGUGCUGGACAGCGACGGCAGCUUCUUC
CUGUACAGCAGACUGACCGUGGACAAGAGCAGAUGGCAAGAGGGCAACGUGUUC
AGCUGCAGCGUGAUGCACGAGGCCCUGCACAACCACUACACACAGAAAAGCCUGA
GCCUGAGCCUGGGCAAGUAAGCUGCCUUCUGCGGGGCUUGCCUUCUGGCCAUGCC
CUUCUUCUCUCCCUUGCACCUGUACCUCUUGGUCUUUGAAUAAAGCCUGAGUAGG
AAGAAAAAAAAAAAAAAAAAAAAAAAAAAAAAAAAAAAAAAAAAAAAAAAAAAA
AAAAAAAAAAAAAAAAAAAAAAAAAAAAA
SEQ ID NO: 129
Pembrolizumab Light Chain (PembrolizumabLC)
AGGAAAUAAGAGAGAAAAGAAGAGUAAGAAGAAAUAUAAGAGCCACCAUGAGGG
CUUGGAUCUUCUUUCUGCUCUGCCUGGCCGGGCGCGCCUUGGCCGAGAUCGUACU
GACACAGAGCCCCGCGACCCUGAGCCUGUCCCCCGGUGAGAGAGCUACCCUGAGC
UGCAGAGCUAGCAAGGGCGUGAGCACAAGCGGCUACAGCUACCUGCACUGGUAUC
AGCAGAAGCCCGGCCAAGCCCCUAGACUGCUGAUCUACCUGGCUAGCUACCUGGA
GAGCGGCGUGCCCGCUAGAUUCAGCGGCAGCGGCAGCGGCACCGACUUCACCCUG
ACCAUCAGCAGCCUGGAGCCCGAGGACUUCGCCGUGUACUACUGUCAGCACAGCA
GAGACCUGCCCCUGACCUUCGGCGGCGGCACCAAGGUGGAGAUCAAGAGAACCGU
GGCCGCCCCUAGCGUGUUCAUCUUCCCCCCUAGCGACGAGCAGCUGAAAAGCGGC
ACCGCUAGCGUGGUGUGCCUGCUGAACAACUUCUACCCUAGAGAGGCCAAGGUGC
AGUGGAAGGUGGACAACGCCCUGCAGAGCGGCAACAGCCAAGAGAGCGUGACCGA
GCAAGACAGCAAGGACAGCACCUACAGCCUGAGCAGCACCCUGACCCUGAGCAAG
GCCGACUACGAGAAGCACAAGGUGUACGCCUGCGAGGUGACCCACCAAGGCCUGA
GCAGCCCCGUGACCAAGAGCUUCAACAGAGGCGAGUGCUAAGCUGCCUUCUGCGG
GGCUUGCCUUCUGGCCAUGCCCUUCUUCUCUCCCUUGCACCUGUACCUCUUGGUC
UUUGAAUAAAGCCUGAGUAGGAAGAAAAAAAAAAAAAAAAAAAAAAAAAAAAAA
AAAAAAAAAAAAAAAAAAAAAAAAAAAAAAAAAAAAAAAAAAAAAAAAAA

We claim:

1. An oligonucleotide bioconjugate of formula (I) 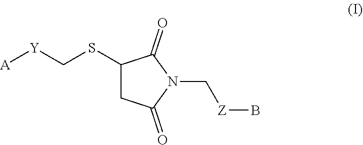
or a pharmaceutically acceptable salt thereof,
wherein
A is an oligonucleotide having a 3′-end and a 5′-end;
Y is a C₁-C₂₀ alkyl, wherein any one or more —CH₂— groups of the C₁-C₂₀ alkyl is each optionally replaced independently with —C(═O)—, —CF₂—, or a heteroatomic moiety selected from the group consisting of —O—, —S—, —NH—, and —N(C₁-C₆ alkyl)-, wherein any one or more —CH₃ groups of the C₁-C₂₀ alkyl is each optionally replaced independently with —CF₃, —CF₂H, —CH₂F, or a heteroatomic moiety selected from the group consisting of —NH(C₁-C₆ alkyl), —N(C₁-C₆ alkyl)₂, —O(C₁-C₆ alkyl), and —S(C₁-C₆ alkyl), and wherein any two heteroatomic moieties are separated from one another by at least two carbon atoms;
Z is a C₁-C₂₀ alkyl, wherein any one or more —CH₂— groups of the C₁-C₂₀ alkyl is each optionally replaced independently with —C(═O)—, —CF₂—, or a heteroatomic moiety selected from the group consisting of —O—, —S—, —NH—, and —N(C₁-C₆ alkyl)-, wherein any one or more —CH₃ groups of the C₁-C₂₀ alkyl is each optionally replaced independently with —CF₃, —CF₂H, —CH₂F, or a heteroatomic moiety selected from the group consisting of —NH(C₁-C₆ alkyl), —N(C₁-C₆ alkyl)₂, —O(C₁-C₆ alkyl), and —S(C₁-C₆ alkyl), and wherein any two heteroatomic moieties are separated from one another by at least two carbon atoms; and
B is an oligonucleotide, a polypeptide, a protein, a small molecule, a carbohydrate, a lipid, a polyethylene glycol (PEG) molecule, or a biopolymer,
wherein Y is covalently attached to the 3′-end of A through a first linkage.

2. The oligonucleotide bioconjugate of claim 1, wherein the first linkage is selected from the group consisting of a phosphate linkage, a phosphorothioate linkage, a phosphoramidate linkage, an amine linkage, an amide linkage, a triazole linkage, an ether linkage, and a thioether linkage.

3. The oligonucleotide bioconjugate of claim 2, wherein the first linkage is a phosphate linkage, and wherein the phosphate linkage between Y and A comprises a phosphate linked to the 3′-oxygen atom of a nucleotide located at the 3′-end of A.

4. The oligonucleotide bioconjugate of any one of claims 1 to 3, wherein A is an mRNA molecule, or wherein A comprises a poly-adenosine monophosphate region or a poly-thymidine monophosphate region.

5. The oligonucleotide bioconjugate of any one of claims 1 to 4, wherein B is an oligonucleotide having a 3′-end and a 5′-end.

6. The oligonucleotide bioconjugate of claim 5, wherein Z is covalently attached to the 3′-end of B through a second linkage selected from the group consisting of a phosphate linkage, a phosphorothioate linkage, a phosphoramidate linkage, an amine linkage, an amide linkage, a triazole linkage, an ether linkage, and a thioether linkage.

7. The oligonucleotide bioconjugate of claim 6, wherein the second linkage is a phosphate linkage, and wherein the phosphate linkage between Z and B comprises a phosphate linked to the 3′-oxygen atom of a nucleotide located at the 3′-end of B.

8. The oligonucleotide bioconjugate of any one of claims 5 to 7, wherein B is an mRNA molecule, or wherein B comprises a poly-adenosine monophosphate region or a poly-thymidine monophosphate region.

9. The oligonucleotide bioconjugate of any one of claims 1 to 4, wherein B is selected from the group consisting of a DNA molecule, a polypeptide, a protein, an antibody, a small molecule, a carbohydrate, a lipid, a PEG molecule, and a biopolymer.

10. The oligonucleotide bioconjugate of any one of claims 1 to 9, wherein
Y is selected from the group consisting of *—CH₂—, *—CH₂CH₂—, *—(CH₂)₂O(CH₂)₂—, and *—(CH₂)₂O(CH₂)₂O(CH₂)₂—, wherein * indicates the attachment point to A; or
Y comprises a polyethylene glycol (PEG) moiety, a polyamide moiety, or an acyl group.

11. The oligonucleotide bioconjugate of any one of claims 1 to 10, wherein
Z is selected from the group consisting of *—CH₂—, *—CH₂CH₂—, *—(CH₂)₂O(CH₂)₂—, and *—(CH₂)₂O(CH₂)₂O(CH₂)₂—, wherein * indicates the attachment point to B; or
Z comprises a polyethylene glycol (PEG) moiety, a polyamide moiety, or an acyl group.

12. An oligonucleotide bioconjugate of formula (II) 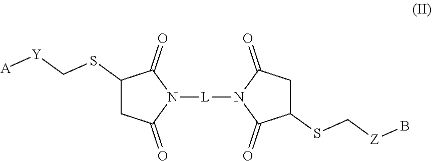
or a pharmaceutically acceptable salt thereof,
wherein
A is an oligonucleotide having a 3′-end and a 5′-end;
Y is a C₁-C₂₀ alkyl, wherein any one or more —CH₂— groups of the C₁-C₂₀ alkyl is each optionally replaced independently with —C(═O)—, —CF₂—, or a heteroatomic moiety selected from the group consisting of —O—, —S—, —NH—, and —N(C₁-C₆ alkyl)-, wherein any one or more —CH₃ groups of the C₁-C₂₀ alkyl is each optionally replaced independently with —CF₃, —CF₂H, —CH₂F, or a heteroatomic moiety selected from the group consisting of —NH(C₁-C₆ alkyl), —N(C₁-C₆ alkyl)₂, —O(C₁-C₆ alkyl), and —S(C₁-C₆ alkyl), and wherein any two heteroatomic moieties are separated from one another by at least two carbon atoms;
L is a C₂-C₅₀ alkyl, C₂-C₅₀ alkenyl, C₂-C₅₀ alkynyl, C₃-C₈ cycloalkyl, aryl, heteroaryl, C₄-C₅₀ alkyl-cycloalkyl, C₇-C₅₀ alkyl-aryl, or C₆-C₅₀ alkyl-heteroaryl, wherein any one or more —CH₂— groups of L is each optionally replaced independently with —C(═O)—, —CF₂—, or a heteroatomic moiety selected from the group consisting of —O—, —S—, —NH—, and —N(C₁-C₆ alkyl)-, wherein any one or more —CH₃ groups of L is each optionally replaced independently with —CF₃, —CF₂H, —CH₂F, or a heteroatomic moiety selected from the group consisting of —NH(C₁-C₆ alkyl), —N(C₁-C₆alkyl)₂, —O(C₁-C₆ alkyl), and —S(C₁-C₆ alkyl), and wherein any two heteroatomic moieties are separated from one another by at least two carbon atoms;
Z is a C₁-C₂₀ alkyl, wherein any one or more —CH₂— groups of the C₁-C₂₀ alkyl is each optionally replaced independently with —C(═O)—, —CF₂—, or a heteroatomic moiety selected from the group consisting of —O—, —S—, —NH—, and —N(C₁-C₆ alkyl)-, wherein any one or more —CH₃ groups of the C₁-C₂₀ alkyl is each optionally replaced independently with —CF₃, —CF₂H, —CH₂F, or a heteroatomic moiety selected from the group consisting of —NH(C₁-C₆ alkyl), —N(C₁-C₆ alkyl)₂, —O(C₁-C₆ alkyl), and —S(C₁-C₆ alkyl), and wherein any two heteroatomic moieties are separated from one another by at least two carbon atoms; and
B is an oligonucleotide, a polypeptide, a protein, a small molecule, a lipid, a carbohydrate, a polyethylene glycol (PEG) molecule, or a biopolymer,
wherein Y is covalently attached to the 3′-end of A through a first linkage.

13. The oligonucleotide bioconjugate of claim 12, wherein the first linkage is selected from the group consisting of a phosphate linkage, a phosphorothioate linkage, a phosphoramidate linkage, an amine linkage, an amide linkage, a triazole linkage, an ether linkage, and a thioether linkage.

14. The oligonucleotide bioconjugate of claim 13, wherein the first linkage is a phosphate linkage, and wherein the phosphate linkage between Y and A comprises a phosphate linked to the 3′-oxygen atom of a nucleotide located at the 3′-end of A.

15. The oligonucleotide bioconjugate of any one of claims 12 to 14, wherein A is an mRNA molecule, or wherein A comprises a poly-adenosine monophosphate region or a poly-thymidine monophosphate region.

16. The oligonucleotide bioconjugate of any one of claims 12 to 15, wherein B is an oligonucleotide having a 3′-end and a 5′-end.

17. The oligonucleotide bioconjugate of claim 16, wherein Z is covalently attached to the 3′-end of B through an second linkage selected from the group consisting of a phosphate linkage, a phosphorothioate linkage, a phosphoramidate linkage, an amine linkage, an amide linkage, a triazole linkage, an ether linkage, and a thioether linkage.

18. The oligonucleotide bioconjugate of claim 17, wherein the second linkage is a phosphate linkage, and wherein the phosphate linkage between Z and B comprises a phosphate linked to the 3′-oxygen atom of a nucleotide located at the 3′-end of B.

19. The oligonucleotide bioconjugate of any one of claims 16 to 18, wherein B is an mRNA molecule, or wherein B comprises a poly-adenosine monophosphate region or a poly-thymidine monophosphate region.

20. The oligonucleotide bioconjugate of any one of claims 12 to 15, wherein B is selected from the group consisting of a DNA molecule, a polypeptide, a protein, an antibody, a small molecule, a carbohydrate, a lipid, a PEG molecule, and a biopolymer.

21. The oligonucleotide bioconjugate of any one of claims 12 to 20, wherein
Y is selected from the group consisting of *—CH₂—, *—CH₂CH₂—, *—(CH₂)₂O(CH₂)₂—, and *—(CH₂)₂O(CH₂)₂O(CH₂)₂—, wherein * indicates the attachment point to A; or
Y comprises a polyethylene glycol (PEG) moiety, a polyamide moiety, or an acyl group.

22. The oligonucleotide bioconjugate of any one of claims 12 to 21, wherein
Z is selected from the group consisting of *—CH₂—, *—CH₂CH₂—, *—(CH₂)₂O(CH₂)₂—, and *—(CH₂)₂O(CH₂)₂O(CH₂)₂—, wherein * indicates the attachment point to B; or
Z comprises a polyethylene glycol (PEG) moiety, a polyamide moiety, or an acyl group.

23. The oligonucleotide bioconjugate of any one of claims 12 to 22, wherein
L is a C₂-C₅₀ alkyl;
L is a polypeptide; or
L comprises a polyethylene glycol (PEG) moiety, a polyamide moiety, an acyl group, or an aryl group.

24. An oligonucleotide bioconjugate of formula (III) 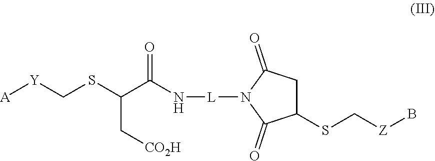
or a pharmaceutically acceptable salt thereof,
wherein
A is an oligonucleotide having a 3′-end and a 5′-end;
Y is a C₁-C₂₀ alkyl, wherein any one or more —CH₂— groups of the C₁-C₂₀ alkyl is each optionally replaced independently with —C(═O)—, —CF₂—, or a heteroatomic moiety selected from the group consisting of —O—, —S—, —NH—, and —N(C₁-C₆ alkyl)-, wherein any one or more —CH₃ groups of the C₁-C₂₀ alkyl is each optionally replaced independently with —CF₃, —CF₂H, —CH₂F, or a heteroatomic moiety selected from the group consisting of —NH(C₁-C₆ alkyl), —N(C₁-C₆ alkyl)₂, —O(C₁-C₆ alkyl), and —S(C₁-C₆ alkyl), and wherein any two heteroatomic moieties are separated from one another by at least two carbon atoms;
L is a C₂-C₅₀ alkyl, C₂-C₅₀ alkenyl, C₂-C₅₀ alkynyl, C₃-C₈ cycloalkyl, aryl, heteroaryl, C₄-C₅₀ alkyl-cycloalkyl, C₇-C₅₀ alkyl-aryl, or C₆-C₅₀ alkyl-heteroaryl, wherein any one or more —CH₂— groups of L is each optionally replaced independently with —C(═O)—, —CF₂—, or a heteroatomic moiety selected from the group consisting of —O—, —S—, —NH—, and —N(C₁-C₆ alkyl)-, wherein any one or more —CH₃ groups of L is each optionally replaced independently with —CF₃, —CF₂H, —CH₂F, or a heteroatomic moiety selected from the group consisting of —NH(C₁-C₆ alkyl), —N(C₁—C₆ alkyl)₂, —O(C₁-C₆ alkyl), and —S(C₁-C₆ alkyl), and wherein any two heteroatomic moieties are separated from one another by at least two carbon atoms;
Z is a C₁-C₂₀ alkyl, wherein any one or more —CH₂— groups of the C₁-C₂₀ alkyl is each optionally replaced independently with —C(═O)—, —CF₂—, or a heteroatomic moiety selected from the group consisting of —O—, —S—, —NH—, and —N(C₁-C₆ alkyl)-, wherein any one or more —CH₃ groups of the C₁-C₂₀ alkyl is each optionally replaced independently with —CF₃, —CF₂H, —CH₂F, or a heteroatomic moiety selected from the group consisting of —NH(C₁-C₆ alkyl), —N(C₁-C₆ alkyl)₂, —O(C₁-C₆ alkyl), and —S(C₁-C₆ alkyl), and wherein any two heteroatomic moieties are separated from one another by at least two carbon atoms; and
B is an oligonucleotide, a polypeptide, a protein, a small molecule, a lipid, a carbohydrate, a polyethylene glycol (PEG) molecule, or a biopolymer,
wherein Y is covalently attached to the 3′-end of A through a first linkage.

25. An oligonucleotide bioconjugate of formula (IV) 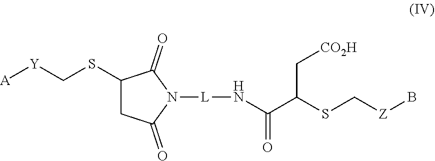
or a pharmaceutically acceptable salt thereof,
wherein
A is an oligonucleotide having a 3′-end and a 5′-end;
Y is a C₁-C₂₀ alkyl, wherein any one or more —CH₂— groups of the C₁-C₂₀ alkyl is each optionally replaced independently with —C(═O)—, —CF₂—, or a heteroatomic moiety selected from the group consisting of —O—, —S—, —NH—, and —N(C₁-C₆ alkyl)-, wherein any one or more —CH₃ groups of the C₁-C₂₀ alkyl is each optionally replaced independently with —CF₃, —CF₂H, —CH₂F, or a heteroatomic moiety selected from the group consisting of —NH(C₁-C₆ alkyl), —N(C₁-C₆ alkyl)₂, —O(C₁-C₆ alkyl), and —S(C₁-C₆ alkyl), and wherein any two heteroatomic moieties are separated from one another by at least two carbon atoms;
L is a C₂-C₅₀ alkyl, C₂-C₅₀ alkenyl, C₂-C₅₀ alkynyl, C₃-C₈ cycloalkyl, aryl, heteroaryl, C₄-C₅₀ alkyl-cycloalkyl, C₇-C₅₀ alkyl-aryl, or C₆-C₅₀ alkyl-heteroaryl, wherein any one or more —CH₂— groups of L is each optionally replaced independently with —C(═O)—, —CF₂—, or a heteroatomic moiety selected from the group consisting of —O—, —S—, —NH—, and —N(C₁-C₆ alkyl)-, wherein any one or more —CH₃ groups of L is each optionally replaced independently with —CF₃, —CF₂H, —CH₂F, or a heteroatomic moiety selected from the group consisting of —NH(C₁-C₆ alkyl), —N(C₁-C₆alkyl)₂, —O(C₁-C₆ alkyl), and —S(C₁-C₆ alkyl), and wherein any two heteroatomic moieties are separated from one another by at least two carbon atoms;
Z is a C₁-C₂₀ alkyl, wherein any one or more —CH₂— groups of the C₁-C₂₀ alkyl is each optionally replaced independently with —C(═O)—, —CF₂—, or a heteroatomic moiety selected from the group consisting of —O—, —S—, —NH—, and —N(C₁-C₆ alkyl)-, wherein any one or more —CH₃ groups of the C₁-C₂₀ alkyl is each optionally replaced independently with —CF₃, —CF₂H, —CH₂F, or a heteroatomic moiety selected from the group consisting of —NH(C₁-C₆ alkyl), —N(C₁-C₆ alkyl)₂, —O(C₁-C₆ alkyl), and —S(C₁-C₆ alkyl), and wherein any two heteroatomic moieties are separated from one another by at least two carbon atoms; and
B is an oligonucleotide, a polypeptide, a protein, a small molecule, a lipid, a carbohydrate, a polyethylene glycol (PEG) molecule, or a biopolymer,
wherein Y is covalently attached to the 3′-end of A through a first linkage.

26. An oligonucleotide bioconjugate of formula (V) 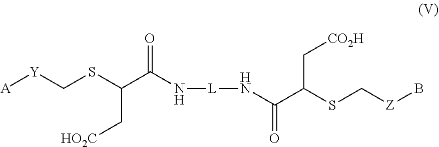
or a pharmaceutically acceptable salt thereof,
wherein
A is an oligonucleotide having a 3′-end and a 5′-end;
Y is a C₁-C₂₀ alkyl, wherein any one or more —CH₂— groups of the C₁-C₂₀ alkyl is each optionally replaced independently with —C(═O)—, —CF₂—, or a heteroatomic moiety selected from the group consisting of —O—, —S—, —NH—, and —N(C₁-C₆ alkyl)-, wherein any one or more —CH₃ groups of the C₁-C₂₀ alkyl is each optionally replaced independently with —CF₃, —CF₂H, —CH₂F, or a heteroatomic moiety selected from the group consisting of —NH(C₁-C₆ alkyl), —N(C₁-C₆ alkyl)₂, —O(C₁-C₆ alkyl), and —S(C₁-C₆ alkyl), and wherein any two heteroatomic moieties are separated from one another by at least two carbon atoms;
L is a C₂-C₅₀ alkyl, C₂-C₅₀ alkenyl, C₂-C₅₀ alkynyl, C₃-C₈ cycloalkyl, aryl, heteroaryl, C₄-C₅₀ alkyl-cycloalkyl, C₇-C₅₀ alkyl-aryl, or C₆-C₅₀ alkyl-heteroaryl, wherein any one or more —CH₂— groups of the L is each optionally replaced independently with —C(═O)—, —CF₂—, or a heteroatomic moiety selected from the group consisting of —O—, —S—, —NH—, and —N(C₁-C₆ alkyl)-, wherein any one or more —CH₃ groups of L is each optionally replaced independently with —CF₃, —CF₂H, —CH₂F, or a heteroatomic moiety selected from the group consisting of —NH(C₁-C₆ alkyl), —N(C₁-C₆alkyl)₂, —O(C₁-C₆ alkyl), and —S(C₁-C₆ alkyl), and wherein any two heteroatomic moieties are separated from one another by at least two carbon atoms;
Z is a C₁-C₂₀ alkyl, wherein any one or more —CH₂— groups of the C₁-C₂₀ alkyl is each optionally replaced independently with —C(═O)—, —CF₂—, or a heteroatomic moiety selected from the group consisting of —O—, —S—, —NH—, and —N(C₁-C₆ alkyl)-, wherein any one or more —CH₃ groups of the C₁-C₂₀ alkyl is each optionally replaced independently with —CF₃, —CF₂H, —CH₂F, or a heteroatomic moiety selected from the group consisting of —NH(C₁-C₆ alkyl), —N(C₁-C₆ alkyl)₂, —O(C₁-C₆ alkyl), and —S(C₁-C₆ alkyl), and wherein any two heteroatomic moieties are separated from one another by at least two carbon atoms; and
B is an oligonucleotide, a polypeptide, a protein, a small molecule, a lipid, a carbohydrate, a polyethylene glycol (PEG) molecule, or a biopolymer,
wherein Y is covalently attached to the 3′-end of A through a first linkage.

27. A pharmaceutical formulation comprising the oligonucleotide bioconjugate of any one of claims 1 to 26, and a pharmaceutically acceptable carrier, excipient, or diluent, optionally wherein the pharmaceutically acceptable carrier, excipient, or diluent comprises a lipid-based carrier, a polymer-based carrier, or a nanocarrier.

28. The pharmaceutical formulation of claim 27, for use in treating, preventing, slowing the progression, or reducing the severity of a disease, disorder, or condition in a patient in need thereof, the disease, disorder, or condition selected from the group consisting of obesity and cancer.

29. The pharmaceutical formulation of claim 27, for use in enzyme replacement therapy (ERT) in a patient in need thereof, optionally wherein the ERT treats one or more lysosomal storage diseases (LSDs).

30. The pharmaceutical formulation of claim 27, for use as a vaccine therapy in a patient in need thereof, optionally wherein the vaccine therapy is for vaccination against an infectious disease.

31. A method of making an oligonucleotide bioconjugate of formula (I) 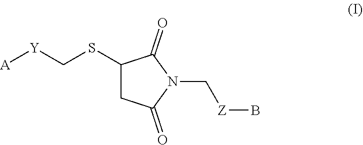
or a pharmaceutically acceptable salt thereof,
wherein
A is an oligonucleotide having a 3′-end and a 5′-end;
Y is a C₁-C₂₀ alkyl, wherein any one or more —CH₂— groups of the C₁-C₂₀ alkyl is each optionally replaced independently with —C(═O)—, —CF₂—, or a heteroatomic moiety selected from the group consisting of —O—, —S—, —NH—, and —N(C₁-C₆ alkyl)-, wherein any one or more —CH₃ groups of the C₁-C₂₀ alkyl is each optionally replaced independently with —CF₃, —CF₂H, —CH₂F, or a heteroatomic moiety selected from the group consisting of —NH(C₁-C₆ alkyl), —N(C₁-C₆ alkyl)₂, —O(C₁-C₆ alkyl), and —S(C₁-C₆ alkyl), and wherein any two heteroatomic moieties are separated from one another by at least two carbon atoms;
Z is a C₁-C₂₀ alkyl, wherein any one or more —CH₂— groups of the C₁-C₂₀ alkyl is each optionally replaced independently with —C(═O)—, —CF₂—, or a heteroatomic moiety selected from the group consisting of —O—, —S—, —NH—, and —N(C₁-C₆ alkyl)-, wherein any one or more —CH₃ groups of the C₁-C₂₀ alkyl is each optionally replaced independently with —CF₃, —CF₂H, —CH₂F, or a heteroatomic moiety selected from the group consisting of —NH(C₁-C₆ alkyl), —N(C₁-C₆ alkyl)₂, —O(C₁-C₆ alkyl), and —S(C₁-C₆ alkyl), and wherein any two heteroatomic moieties are separated from one another by at least two carbon atoms; and
B is an oligonucleotide, a polypeptide, a protein, a small molecule, a carbohydrate, a lipid, a polyethylene glycol (PEG) molecule, or a biopolymer,
wherein Y is covalently attached to the 3′-end of A through a first linkage, the method comprising a step of reacting the thiol of formula (VI) with the maleimide of formula (VII) to form the oligonucleotide bioconjugate of formula (I) 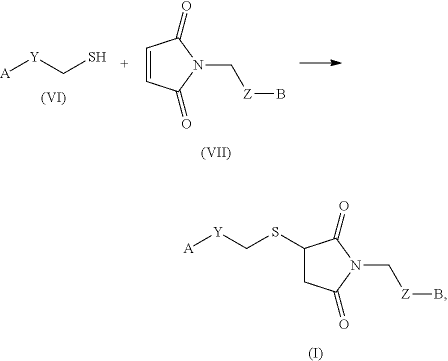
wherein A and Y of formula (VI) are defined as above for formula (I), and wherein Z and B of formula (VII) are defined as above for formula (I).

32. A method of making an oligonucleotide bioconjugate of formula (II) 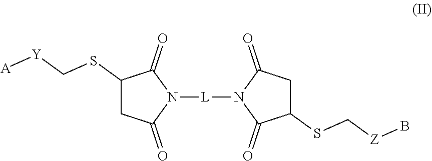
or a pharmaceutically acceptable salt thereof,
wherein
A is an oligonucleotide having a 3′-end and a 5′-end;
Y is a C₁-C₂₀ alkyl, wherein any one or more —CH₂—groups of the C₁-C₂₀ alkyl is each optionally replaced independently with —C(═O)—, —CF₂—, or a heteroatomic moiety selected from the group consisting of —O—, —S—, —NH—, and —N(C₁-C₆ alkyl)-, wherein any one or more —CH₃ groups of the C₁-C₂₀ alkyl is each optionally replaced independently with —CF₃, —CF₂H, —CH₂F, or a heteroatomic moiety selected from the group consisting of —NH(C₁-C₆ alkyl), —N(C₁-C₆ alkyl)₂, —O(C₁-C₆ alkyl), and —S(C₁-C₆ alkyl), and wherein any two heteroatomic moieties are separated from one another by at least two carbon atoms;
L is a C₂-C₅₀ alkyl, C₂-C₅₀ alkenyl, C₂-C₅₀ alkynyl, C₃-C₈ cycloalkyl, aryl, heteroaryl, C₄-C₅₀ alkyl-cycloalkyl, C₇-C₅₀ alkyl-aryl, or C₆-C₅₀ alkyl-heteroaryl, wherein any one or more —CH₂— groups of L is each optionally replaced independently with —C(═O)—, —CF₂—, or a heteroatomic moiety selected from the group consisting of —O—, —S—, —NH—, and —N(C₁-C₆ alkyl)-, wherein any one or more —CH₃ groups of L is each optionally replaced independently with —CF₃, —CF₂H, —CH₂F, or a heteroatomic moiety selected from the group consisting of —NH(C₁-C₆ alkyl), —N(C₁-C₆alkyl)₂, —O(C₁-C₆ alkyl), and —S(C₁-C₆ alkyl), and wherein any two heteroatomic moieties are separated from one another by at least two carbon atoms;
Z is a C₁-C₂₀ alkyl, wherein any one or more —CH₂— groups of the C₁-C₂₀ alkyl is each optionally replaced independently with —C(═O)—, —CF₂—, or a heteroatomic moiety selected from the group consisting of —O—, —S—, —NH—, and —N(C₁-C₆ alkyl)-, wherein any one or more —CH₃ groups of the C₁-C₂₀ alkyl is each optionally replaced independently with —CF₃, —CF₂H, —CH₂F, or a heteroatomic moiety selected from the group consisting of —NH(C₁-C₆ alkyl), —N(C₁-C₆ alkyl)₂, —O(C₁-C₆ alkyl), and —S(C₁-C₆ alkyl), and wherein any two heteroatomic moieties are separated from one another by at least two carbon atoms; and
B is an oligonucleotide, a polypeptide, a protein, a small molecule, a lipid, a carbohydrate, a polyethylene glycol (PEG) molecule, or a biopolymer,
wherein Y is covalently attached to the 3′-end of A through a first linkage,
the method comprising the steps of
(i) reacting the thiol of formula (VI) with one of the two maleimides of formula (VIII) to form a compound of formula (IX) 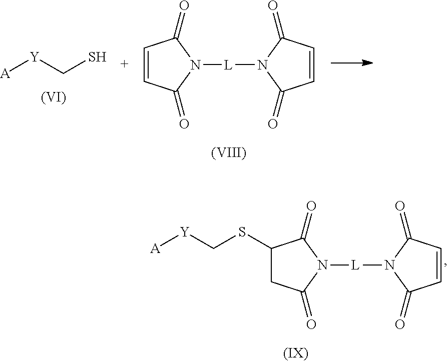
wherein A and Y of formula (VI) are defined as above for formula (II), and wherein L of formula (VIII) is defined as above for formula (II), and
(ii) reacting the maleimide of formula (IX) with the thiol of formula (X) to form the oligonucleotide bioconjugate of formula (II) 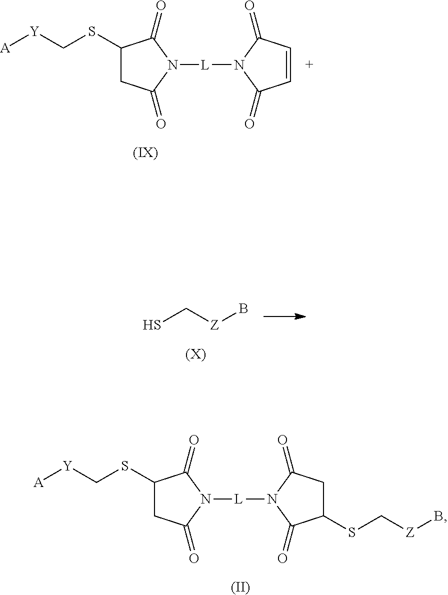
wherein Z and B of formula (X) are as defined above for formula (II).

33. A method of making an oligonucleotide bioconjugate of formula (III) 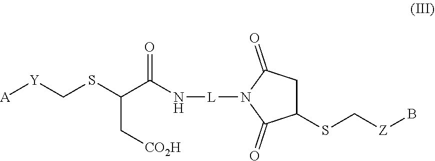
or a pharmaceutically acceptable salt thereof,
wherein
A is an oligonucleotide having a 3′-end and a 5′-end;
Y is a C₁-C₂₀ alkyl, wherein any one or more —CH₂—groups of the C₁-C₂₀ alkyl is each optionally replaced independently with —C(═O)—, —CF₂—, or a heteroatomic moiety selected from the group consisting of —O—, —S—, —NH—, and —N(C₁—C alkyl)-, wherein any one or more —CH₃ groups of the C₁-C₂₀ alkyl is each optionally replaced independently with —CF₃, —CF₂H, —CH₂F, or a heteroatomic moiety selected from the group consisting of —NH(C₁-C₆ alkyl), —N(C₁-C₆ alkyl)₂, —O(C₁-C₆ alkyl), and —S(C₁-C₆ alkyl), and wherein any two heteroatomic moieties are separated from one another by at least two carbon atoms;
L is a C₂-C₅₀ alkyl, C₂-C₅₀ alkenyl, C₂-C₅₀ alkynyl, C₃-C₈ cycloalkyl, aryl, heteroaryl, C₄-C₅₀ alkyl-cycloalkyl, C₇-C₅₀ alkyl-aryl, or C₆-C₅₀ alkyl-heteroaryl, wherein any one or more —CH₂— groups of L is each optionally replaced independently with —C(═O)—, —CF₂—, or a heteroatomic moiety selected from the group consisting of —O—, —S—, —NH—, and —N(C₁-C₆ alkyl)-, wherein any one or more —CH₃ groups of L is each optionally replaced independently with —CF₃, —CF₂H, —CH₂F, or a heteroatomic moiety selected from the group consisting of —NH(C₁-C₆ alkyl), —N(C₁-C₆alkyl)₂, —O(C₁-C₆ alkyl), and —S(C₁-C₆ alkyl), and wherein any two heteroatomic moieties are separated from one another by at least two carbon atoms;
Z is a C₁-C₂₀ alkyl, wherein any one or more —CH₂— groups of the C₁-C₂₀ alkyl is each optionally replaced independently with —C(═O)—, —CF₂—, or a heteroatomic moiety selected from the group consisting of —O—, —S—, —NH—, and —N(C₁-C₆ alkyl)-, wherein any one or more —CH₃ groups of the C₁-C₂₀ alkyl is each optionally replaced independently with —CF₃, —CF₂H, —CH₂F, or a heteroatomic moiety selected from the group consisting of —NH(C₁-C₆ alkyl), —N(C₁-C₆ alkyl)₂, —O(C₁-C₆ alkyl), and —S(C₁-C₆ alkyl), and wherein any two heteroatomic moieties are separated from one another by at least two carbon atoms; and
B is an oligonucleotide, a polypeptide, a protein, a small molecule, a lipid, a carbohydrate, a polyethylene glycol (PEG) molecule, or a biopolymer,
wherein Y is covalently attached to the 3′-end of A through a first linkage,
the method comprising the steps of
(i) reacting the thiol of formula (VI) with one of the two maleimides of formula (VIII) to form a compound of formula (IX) 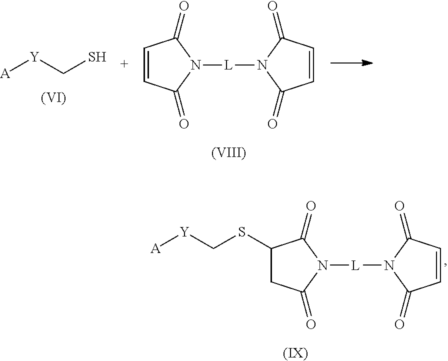
wherein A and Y of formula (VI) are defined as above for formula (III), and wherein L of formula (VIII) is defined as above for formula (III),
(ii) reacting the maleimide of formula (IX) with the thiol of formula (X) to form a compound of formula (II) 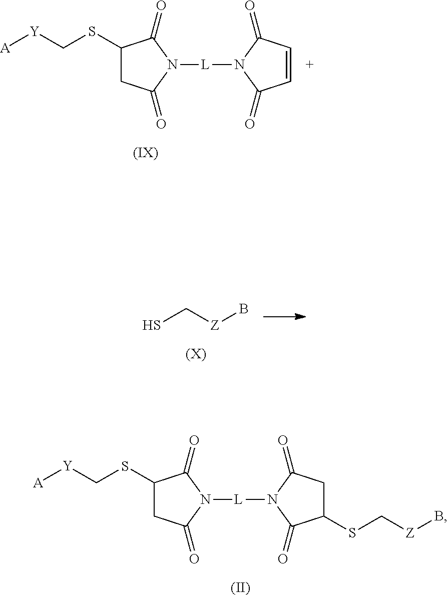
wherein Z and B of formula (X) are as defined above for formula (III), and
(iii) hydrolyzing one of the succinimides of formula (II) to form the oligonucleotide bioconjugate of formula (III)

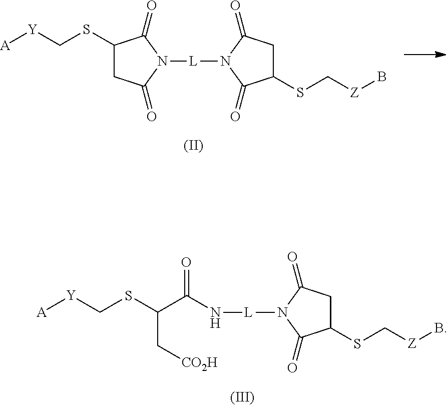

34. A method of making an oligonucleotide bioconjugate of formula (IV) 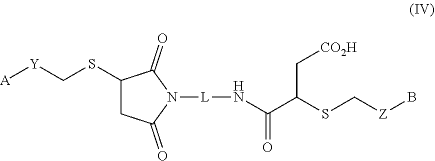
or a pharmaceutically acceptable salt thereof,
wherein
A is an oligonucleotide having a 3′-end and a 5′-end;
Y is a C₁-C₂₀ alkyl, wherein any one or more —CH₂— groups of the C₁-C₂₀ alkyl is each optionally replaced independently with —C(═O)—, —CF₂—, or a heteroatomic moiety selected from the group consisting of —O—, —S—, —NH—, and —N(C₁-C₆ alkyl)-, wherein any one or more —CH₃ groups of the C₁-C₂₀ alkyl is each optionally replaced independently with —CF₃, —CF₂H, —CH₂F, or a heteroatomic moiety selected from the group consisting of —NH(C₁-C₆ alkyl), —N(C₁-C₆ alkyl)₂, —O(C₁-C₆ alkyl), and —S(C₁-C₆ alkyl), and wherein any two heteroatomic moieties are separated from one another by at least two carbon atoms;
L is a C₂-C₅₀ alkyl, C₂-C₅₀ alkenyl, C₂-C₅₀ alkynyl, C₃-C₈ cycloalkyl, aryl, heteroaryl, C₄-C₅₀ alkyl-cycloalkyl, C₇-C₅₀ alkyl-aryl, or C₆-C₅₀ alkyl-heteroaryl, wherein any one or more —CH₂— groups of L is each optionally replaced independently with —C(═O)—, —CF₂—, or a heteroatomic moiety selected from the group consisting of —O—, —S—, —NH—, and —N(C₁-C₆ alkyl)-, wherein any one or more —CH₃ groups of L is each optionally replaced independently with —CF₃, —CF₂H, —CH₂F, or a heteroatomic moiety selected from the group consisting of —NH(C₁-C₆ alkyl), —N(C₁-C₆alkyl)₂, —O(C₁-C₆ alkyl), and —S(C₁-C₆ alkyl), and wherein any two heteroatomic moieties are separated from one another by at least two carbon atoms;
Z is a C₁-C₂₀ alkyl, wherein any one or more —CH₂— groups of the C₁-C₂₀ alkyl is each optionally replaced independently with —C(═O)—, —CF₂—, or a heteroatomic moiety selected from the group consisting of —O—, —S—, —NH—, and —N(C₁-C₆ alkyl)-, wherein any one or more —CH₃ groups of the C₁-C₂₀ alkyl is each optionally replaced independently with —CF₃, —CF₂H, —CH₂F, or a heteroatomic moiety selected from the group consisting of —NH(C₁-C₆ alkyl), —N(C₁-C₆ alkyl)₂, —O(C₁-C₆ alkyl), and —S(C₁-C₆ alkyl), and wherein any two heteroatomic moieties are separated from one another by at least two carbon atoms; and
B is an oligonucleotide, a polypeptide, a protein, a small molecule, a lipid, a carbohydrate, a polyethylene glycol (PEG) molecule, or a biopolymer,
wherein Y is covalently attached to the 3′-end of A through a first linkage,
the method comprising the steps of
(i) reacting the thiol of formula (VI) with the maleimide of formula (VIII) to form a compound of formula (IX) 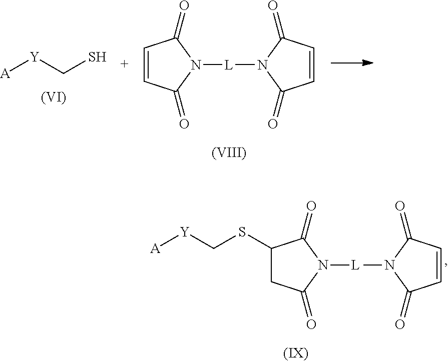
wherein A and Y of formula (VI) are defined as above for formula (IV), and wherein
L of formula (VIII) is defined as above for formula (IV),
(ii) reacting the maleimide of formula (IX) with the thiol of formula (X) to form a compound of formula (II) 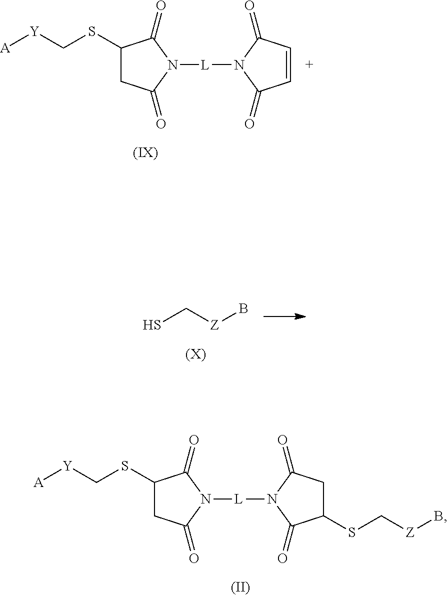
wherein Z and B of formula (X) are as defined above for formula (IV), and
(iii) hydrolyzing one of the succinimides of formula (II) to form the oligonucleotide bioconjugate of formula (IV)

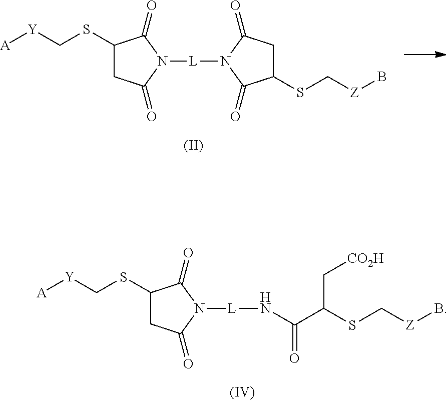

35. A method of making an oligonucleotide bioconjugate of formula (V) 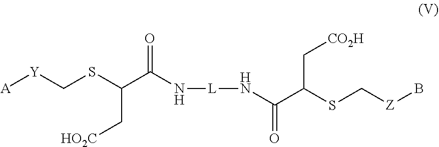
or a pharmaceutically acceptable salt thereof,
wherein
A is an oligonucleotide having a 3′-end and a 5′-end;
Y is a C₁-C₂₀ alkyl, wherein any one or more —CH₂— groups of the C₁-C₂₀ alkyl is each optionally replaced independently with —C(═O)—, —CF₂—, or a heteroatomic moiety selected from the group consisting of —O—, —S—, —NH—, and —N(C₁-C₆ alkyl)-, wherein any one or more —CH₃ groups of the C₁-C₂₀ alkyl is each optionally replaced independently with —CF₃, —CF₂H, —CH₂F, or a heteroatomic moiety selected from the group consisting of —NH(C₁-C₆ alkyl), —N(C₁-C₆ alkyl)₂, —O(C₁-C₆ alkyl), and —S(C₁-C₆ alkyl), and wherein any two heteroatomic moieties are separated from one another by at least two carbon atoms;
L is a C₂-C₅₀ alkyl, C₂-C₅₀ alkenyl, C₂-C₅₀ alkynyl, C₃-C₈ cycloalkyl, aryl, heteroaryl, C₄-C₅₀ alkyl-cycloalkyl, C₇-C₅₀ alkyl-aryl, or C₆-C₅₀ alkyl-heteroaryl, wherein any one or more —CH₂— groups of L is each optionally replaced independently with —C(═O)—, —CF₂—, or a heteroatomic moiety selected from the group consisting of —O—, —S—, —NH—, and —N(C₁-C₆ alkyl)-, wherein any one or more —CH₃ groups of L is each optionally replaced independently with —CF₃, —CF₂H, —CH₂F, or a heteroatomic moiety selected from the group consisting of —NH(C₁-C₆ alkyl), —N(C₁-C₆alkyl)₂, —O(C₁-C₆ alkyl), and —S(C₁-C₆ alkyl), and wherein any two heteroatomic moieties are separated from one another by at least two carbon atoms;
Z is a C₁-C₂₀ alkyl, wherein any one or more —CH₂— groups of the C₁-C₂₀ alkyl is each optionally replaced independently with —C(═O)—, —CF₂—, or a heteroatomic moiety selected from the group consisting of —O—, —S—, —NH—, and —N(C₁-C₆ alkyl)-, wherein any two heteroatomic moieties are separated from one another by at least two carbon atoms; and
B is an oligonucleotide, a polypeptide, a protein, a small molecule, a lipid, a carbohydrate, a polyethylene glycol (PEG) molecule, or a biopolymer,
wherein Y is covalently attached to the 3′-end of A through a first linkage,
the method comprising the steps of
(i) reacting the thiol of formula (VI) with one of the two maleimides of formula (VIII) to form a compound of formula (IX) 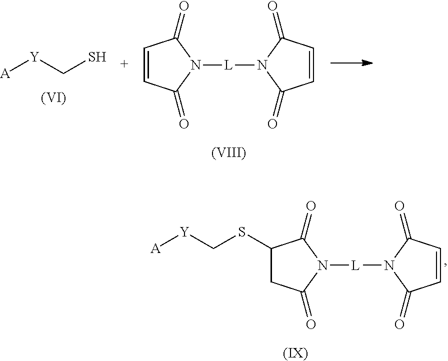
wherein A and Y of formula (VI) are defined as above for formula (V), and wherein
L is defined as above for formula (V),
(ii) reacting the maleimide of formula (IX) with the thiol of formula (X) to form a compound of formula (II) 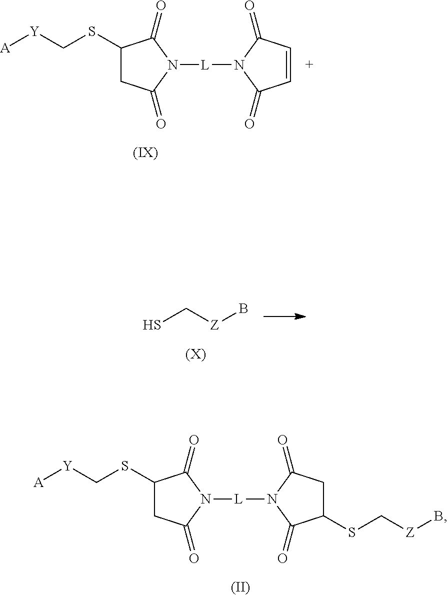
wherein Z and B of formula (X) are as defined above for formula (V), and
(iii) hydrolyzing both of the succinimides of formula (II) to form the oligonucleotide bioconjugate of formula (V)

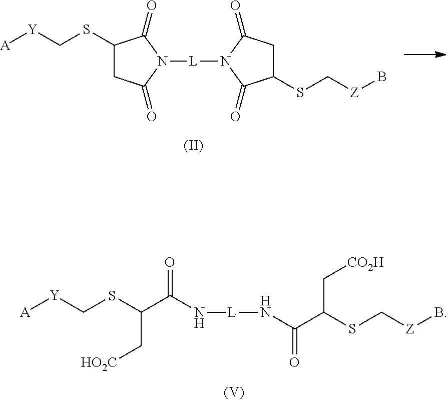

36. A method of co-expressing a first polypeptide and a second polypeptide in a cell, the method comprising a step of contacting the cell with an mRNA bioconjugate of formula (I) 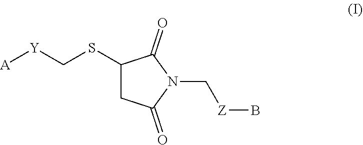
or a pharmaceutically acceptable salt thereof,
wherein
A is a first mRNA molecule having a 3′-end and a 5′-end, wherein A encodes the first polypeptide;
Y is a C₁-C₂₀ alkyl, wherein any one or more —CH₂— groups of the C₁-C₂₀ alkyl is each optionally replaced independently with —C(═O)—, —CF₂—, or a heteroatomic moiety selected from the group consisting of —O—, —S—, —NH—, and —N(C₁-C₆ alkyl)-, wherein any one or more —CH₃ groups of the C₁-C₂₀ alkyl is each optionally replaced independently with —CF₃, —CF₂H, —CH₂F, or a heteroatomic moiety selected from the group consisting of —NH(C₁-C₆ alkyl), —N(C₁-C₆ alkyl)₂, —O(C₁-C₆ alkyl), and —S(C₁-C₆ alkyl), and wherein any two heteroatomic moieties are separated from one another by at least two carbon atoms;
Z is a C₁-C₂₀ alkyl, wherein any one or more —CH₂— groups of the C₁-C₂₀ alkyl is each optionally replaced independently with —C(═O)—, —CF₂—, or a heteroatomic moiety selected from the group consisting of —O—, —S—, —NH—, and —N(C₁-C₆ alkyl)-, wherein any one or more —CH₃ groups of the C₁-C₂₀ alkyl is each optionally replaced independently with —CF₃, —CF₂H, —CH₂F, or a heteroatomic moiety selected from the group consisting of —NH(C₁-C₆ alkyl), —N(C₁-C₆ alkyl)₂, —O(C₁-C₆ alkyl), and —S(C₁-C₆ alkyl), and wherein any two heteroatomic moieties are separated from one another by at least two carbon atoms; and
B is a second mRNA molecule having a 3′-end and a 5′-end, wherein B encodes the second polypeptide,
wherein Y is covalently attached to the 3′-end of A through a first linkage, and wherein Z is covalently attached to the 3′-end of B through a second linkage.

37. A method of co-expressing a first polypeptide and a second polypeptide in a cell, the method comprising a step of contacting the cell with an mRNA bioconjugate of formula (II) 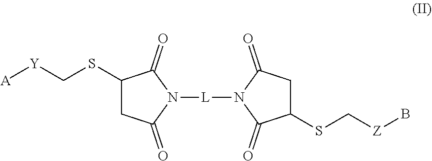
or a pharmaceutically acceptable salt thereof,
wherein
A is a first mRNA molecule having a 3′-end and a 5′-end, wherein A encodes the first polypeptide;
Y is a C₁-C₂₀ alkyl, wherein any one or more —CH₂— groups of the C₁-C₂₀ alkyl is each optionally replaced independently with —C(═O)—, —CF₂—, or a heteroatomic moiety selected from the group consisting of —O—, —S—, —NH—, and —N(C₁-C₆ alkyl)-, wherein any one or more —CH₃ groups of the C₁-C₂₀ alkyl is each optionally replaced independently with —CF₃, —CF₂H, —CH₂F, or a heteroatomic moiety selected from the group consisting of —NH(C₁-C₆ alkyl), —N(C₁-C₆ alkyl)₂, —O(C₁-C₆ alkyl), and —S(C₁-C₆ alkyl), and wherein any two heteroatomic moieties are separated from one another by at least two carbon atoms;
L is a C₂-C₅₀ alkyl, C₂-C₅₀ alkenyl, C₂-C₅₀ alkynyl, C₃-C₈ cycloalkyl, aryl, heteroaryl, C₄-C₅₀ alkyl-cycloalkyl, C₇-C₅₀ alkyl-aryl, or C₆-C₅₀ alkyl-heteroaryl, wherein any one or more —CH₂— groups of L is each optionally replaced independently with —C(═O)—, —CF₂—, or a heteroatomic moiety selected from the group consisting of —O—, —S—, —NH—, and —N(C₁-C₆ alkyl)-, wherein any one or more —CH₃ groups of L is each optionally replaced independently with —CF₃, —CF₂H, —CH₂F, or a heteroatomic moiety selected from the group consisting of —NH(C₁-C₆ alkyl), —N(C₁-C₆ alkyl)₂, —O(C₁-C₆ alkyl), and —S(C₁-C₆ alkyl), and wherein any two heteroatomic moieties are separated from one another by at least two carbon atoms;
Z is a C₁-C₂₀ alkyl, wherein any one or more —CH₂— groups of the C₁-C₂₀ alkyl is each optionally replaced independently with —C(═O)—, —CF₂—, or a heteroatomic moiety selected from the group consisting of —O—, —S—, —NH—, and —N(C₁-C₆ alkyl)-, wherein any one or more —CH₃ groups of the C₁-C₂₀ alkyl is each optionally replaced independently with —CF₃, —CF₂H, —CH₂F, or a heteroatomic moiety selected from the group consisting of —NH(C₁-C₆ alkyl), —N(C₁-C₆ alkyl)₂, —O(C₁-C₆ alkyl), and —S(C₁-C₆ alkyl), and wherein any two heteroatomic moieties are separated from one another by at least two carbon atoms; and
B is a second mRNA molecule having a 3′-end and a 5′-end, wherein B encodes the second polypeptide;
wherein Y is covalently attached to the 3′-end of A through a first linkage, and wherein Z is covalently attached to the 3′-end of B through a second linkage.

38. A method of delivering equimolar amounts of a first mRNA molecule and a second mRNA molecule to a cell, the method comprising a step of contacting the cell with an mRNA bioconjugate of formula (I) 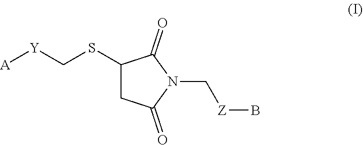
or a pharmaceutically acceptable salt thereof,
wherein
A is a first mRNA molecule having a 3′-end and a 5′-end;
Y is a C₁-C₂₀ alkyl, wherein any one or more —CH₂— groups of the C₁-C₂₀ alkyl is each optionally replaced independently with —C(═O)—, —CF₂—, or a heteroatomic moiety selected from the group consisting of —O—, —S—, —NH—, and —N(C₁-C₆ alkyl)-, wherein any one or more —CH₃ groups of the C₁-C₂₀ alkyl is each optionally replaced independently with —CF₃, —CF₂H, —CH₂F, or a heteroatomic moiety selected from the group consisting of —NH(C₁-C₆ alkyl), —N(C₁-C₆ alkyl)₂, —O(C₁-C₆ alkyl), and —S(C₁-C₆ alkyl), and wherein any two heteroatomic moieties are separated from one another by at least two carbon atoms;
Z is a C₁-C₂₀ alkyl, wherein any one or more —CH₂— groups of the C₁-C₂₀ alkyl is each optionally replaced independently with —C(═O)—, —CF₂—, or a heteroatomic moiety selected from the group consisting of —O—, —S—, —NH—, and —N(C₁-C₆ alkyl)-, wherein any one or more —CH₃ groups of the C₁-C₂₀ alkyl is each optionally replaced independently with —CF₃, —CF₂H, —CH₂F, or a heteroatomic moiety selected from the group consisting of —NH(C₁-C₆ alkyl), —N(C₁-C₆ alkyl)₂, —O(C₁-C₆ alkyl), and —S(C₁-C₆ alkyl), and wherein any two heteroatomic moieties are separated from one another by at least two carbon atoms; and
B is a second mRNA molecule having a 3′-end and a 5′-end,
wherein Y is covalently attached to the 3′-end of A through a first linkage, and wherein Z is covalently attached to the 3′-end of B through a second linkage.

39. A method of delivering equimolar amounts of a first mRNA molecule and a second mRNA molecule to a cell, the method comprising a step of contacting the cell with an mRNA bioconjugate of formula (II) 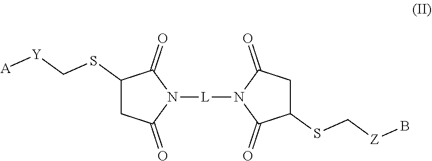
or a pharmaceutically acceptable salt thereof,
wherein
A is a first mRNA molecule having a 3′-end and a 5′-end;
Y is a C₁-C₂₀ alkyl, wherein any one or more —CH₂— groups of the C₁-C₂₀ alkyl is each optionally replaced independently with —C(═O)—, —CF₂—, or a heteroatomic moiety selected from the group consisting of —O—, —S—, —NH—, and —N(C₁-C₆ alkyl)-, wherein any one or more —CH₃ groups of the C₁-C₂₀ alkyl is each optionally replaced independently with —CF₃, —CF₂H, —CH₂F, or a heteroatomic moiety selected from the group consisting of —NH(C₁-C₆ alkyl), —N(C₁-C₆ alkyl)₂, —O(C₁-C₆ alkyl), and —S(C₁-C₆ alkyl), and wherein any two heteroatomic moieties are separated from one another by at least two carbon atoms;
L is a C₂-C₅₀ alkyl, C₂-C₅₀ alkenyl, C₂-C₅₀ alkynyl, C₃-C₈ cycloalkyl, aryl, heteroaryl, C₄-C₅₀ alkyl-cycloalkyl, C₇-C₅₀ alkyl-aryl, or C₆-C₅₀ alkyl-heteroaryl, wherein any one or more —CH₂— groups of L is each optionally replaced independently with —C(═O)—, —CF₂—, or a heteroatomic moiety selected from the group consisting of —O—, —S—, —NH—, and —N(C₁-C₆ alkyl)-, wherein any one or more —CH₃ groups of L is each optionally replaced independently with —CF₃, —CF₂H, —CH₂F, or a heteroatomic moiety selected from the group consisting of —NH(C₁-C₆ alkyl), —N(C₁-C₆alkyl)₂, —O(C₁-C₆ alkyl), and —S(C₁-C₆ alkyl), and wherein any two heteroatomic moieties are separated from one another by at least two carbon atoms;
Z is a C₁-C₂₀ alkyl, wherein any one or more —CH₂— groups of the C₁-C₂₀ alkyl is each optionally replaced independently with —C(═O)—, —CF₂—, or a heteroatomic moiety selected from the group consisting of —O—, —S—, —NH—, and —N(C₁-C₆ alkyl)-, wherein any one or more —CH₃ groups of the C₁-C₂₀ alkyl is each optionally replaced independently with —CF₃, —CF₂H, —CH₂F, or a heteroatomic moiety selected from the group consisting of —NH(C₁-C₆ alkyl), —N(C₁-C₆ alkyl)₂, —O(C₁-C₆ alkyl), and —S(C₁-C₆ alkyl), and wherein any two heteroatomic moieties are separated from one another by at least two carbon atoms; and
B is a second mRNA molecule having a 3′-end and a 5′-end;
wherein Y is covalently attached to the 3′-end of A through a first linkage, and wherein Z is covalently attached to the 3′-end of B through a second linkage.

40. A method of targeted therapy, the method comprising a step of administering to a patient in need thereof a pharmaceutical formulation comprising an mRNA bioconjugate of formula (I) 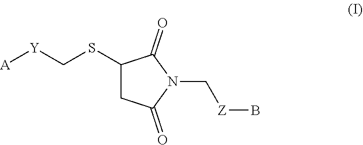
or a pharmaceutically acceptable salt thereof, and a pharmaceutically acceptable carrier, excipient, or diluent,
wherein
A is a first mRNA molecule having a 3′-end and a 5′-end, wherein A encodes a first therapeutic polypeptide;
Y is a C₁-C₂₀ alkyl, wherein any one or more —CH₂— groups of the C₁-C₂₀ alkyl is each optionally replaced independently with —C(═O)—, —CF₂—, or a heteroatomic moiety selected from the group consisting of —O—, —S—, —NH—, and —N(C₁-C₆ alkyl)-, wherein any one or more —CH₃ groups of the C₁-C₂₀ alkyl is each optionally replaced independently with —CF₃, —CF₂H, —CH₂F, or a heteroatomic moiety selected from the group consisting of —NH(C₁-C₆ alkyl), —N(C₁-C₆ alkyl)₂, —O(C₁-C₆ alkyl), and —S(C₁-C₆ alkyl), and wherein any two heteroatomic moieties are separated from one another by at least two carbon atoms;
Z is a C₁-C₂₀ alkyl, wherein any one or more —CH₂— groups of the C₁-C₂₀ alkyl is each optionally replaced independently with —C(═O)—, —CF₂—, or a heteroatomic moiety selected from the group consisting of —O—, —S—, —NH—, and —N(C₁-C₆ alkyl)-, wherein any one or more —CH₃ groups of the C₁-C₂₀ alkyl is each optionally replaced independently with —CF₃, —CF₂H, —CH₂F, or a heteroatomic moiety selected from the group consisting of —NH(C₁-C₆ alkyl), —N(C₁-C₆ alkyl)₂, —O(C₁-C₆ alkyl), and —S(C₁-C₆ alkyl), and wherein any two heteroatomic moieties are separated from one another by at least two carbon atoms; and
B is a second therapeutic polypeptide, a therapeutic small molecule, or a second mRNA molecule having a 3′-end and a 5′-end encoding a second therapeutic polypeptide,
wherein Y is covalently attached to the 3′-end of A through a first linkage.

41. The method of claim 40, wherein the targeted therapy delivers the mRNA bioconjugate to a specific cell type, organ, tumor, or anatomical location in the patient in need thereof.

42. The method of claim 40 or claim 41, further comprising a step of co-expressing the first and second therapeutic polypeptides.

43. The method of any one of claims 40 to 42, wherein the targeted therapy is targeted cancer therapy or targeted obesity therapy.

44. A method of targeted therapy, the method comprising a step of administering to a patient in need thereof a pharmaceutical formulation comprising an mRNA bioconjugate of formula (II) 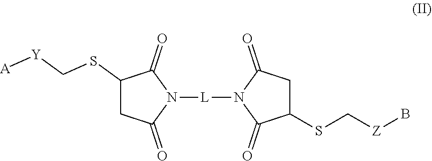
or a pharmaceutically acceptable salt thereof, and a pharmaceutically acceptable carrier, excipient, or diluent,
wherein
A is a first mRNA molecule having a 3′-end and a 5′-end, wherein A encodes a first therapeutic polypeptide;
Y is a C₁-C₂₀ alkyl, wherein any one or more —CH₂— groups of the C₁-C₂₀ alkyl is each optionally replaced independently with —C(═O)—, —CF₂—, or a heteroatomic moiety selected from the group consisting of —O—, —S—, —NH—, and —N(C₁-C₆ alkyl)-, wherein any one or more —CH₃ groups of the C₁-C₂₀ alkyl is each optionally replaced independently with —CF₃, —CF₂H, —CH₂F, or a heteroatomic moiety selected from the group consisting of —NH(C₁-C₆ alkyl), —N(C₁-C₆ alkyl)₂, —O(C₁-C₆ alkyl), and —S(C₁-C₆ alkyl), and wherein any two heteroatomic moieties are separated from one another by at least two carbon atoms;
L is a C₂-C₅₀ alkyl, C₂-C₅₀ alkenyl, C₂-C₅₀ alkynyl, C₃-C₈ cycloalkyl, aryl, heteroaryl, C₄-C₅₀ alkyl-cycloalkyl, C₇-C₅₀ alkyl-aryl, or C₆-C₅₀ alkyl-heteroaryl, wherein any one or more —CH₂— groups of L is each optionally replaced independently with —C(═O)—, —CF₂—, or a heteroatomic moiety selected from the group consisting of —O—, —S—, —NH—, and —N(C₁-C₆ alkyl)-, wherein any one or more —CH₃ groups of L is each optionally replaced independently with —CF₃, —CF₂H, —CH₂F, or a heteroatomic moiety selected from the group consisting of —NH(C₁-C₆ alkyl), —N(C₁-C₆ alkyl)₂, —O(C₁-C₆ alkyl), and —S(C₁-C₆ alkyl), and wherein any two heteroatomic moieties are separated from one another by at least two carbon atoms;
Z is a C₁-C₂₀ alkyl, wherein any one or more —CH₂— groups of the C₁-C₂₀ alkyl is each optionally replaced independently with —C(═O)—, —CF₂—, or a heteroatomic moiety selected from the group consisting of —O—, —S—, —NH—, and —N(C₁-C₆ alkyl)-, wherein any one or more —CH₃ groups of the C₁-C₂₀ alkyl is each optionally replaced independently with —CF₃, —CF₂H, —CH₂F, or a heteroatomic moiety selected from the group consisting of —NH(C₁-C₆ alkyl), —N(C₁-C₆ alkyl)₂, —O(C₁-C₆ alkyl), and —S(C₁-C₆ alkyl), and wherein any two heteroatomic moieties are separated from one another by at least two carbon atoms; and
B is a second therapeutic polypeptide, a therapeutic small molecule, or a second mRNA molecule having a 3′-end and a 5′-end encoding a second therapeutic polypeptide,
wherein Y is covalently attached to the 3′-end of A through a first linkage.

45. The method of claim 44, wherein the targeted therapy delivers the mRNA bioconjugate to a specific cell type, organ, tumor, or anatomical location in the patient in need thereof.

46. The method of claim 44 or claim 45, further comprising a step of co-expressing the first and second therapeutic polypeptides.

47. The method of any one of claims 44 to 46, wherein the targeted therapy is targeted cancer therapy or targeted obesity therapy.

48. A method of treating, preventing, slowing the progression, or reducing the severity of cancer in a patient in need thereof, the method comprising a step of administering to the patient in need thereof a pharmaceutical formulation comprising an mRNA bioconjugate of formula (I) 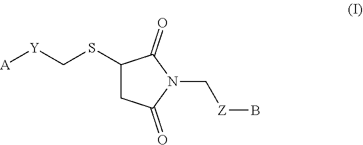
or a pharmaceutically acceptable salt thereof, and a pharmaceutically acceptable carrier, excipient, or diluent,
wherein
A is a first mRNA molecule having a 3′-end and a 5′-end, wherein A encodes a first therapeutic polypeptide;
Y is a C₁-C₂₀ alkyl, wherein any one or more —CH₂— groups of the C₁-C₂₀ alkyl is each optionally replaced independently with —C(═O)—, —CF₂—, or a heteroatomic moiety selected from the group consisting of —O—, —S—, —NH—, and —N(C₁-C₆ alkyl)-, wherein any one or more —CH₃ groups of the C₁-C₂₀ alkyl is each optionally replaced independently with —CF₃, —CF₂H, —CH₂F, or a heteroatomic moiety selected from the group consisting of —NH(C₁-C₆ alkyl), —N(C₁-C₆ alkyl)₂, —O(C₁-C₆ alkyl), and —S(C₁-C₆ alkyl), and wherein any two heteroatomic moieties are separated from one another by at least two carbon atoms;
Z is a C₁-C₂₀ alkyl, wherein any one or more —CH₂— groups of the C₁-C₂₀ alkyl is each optionally replaced independently with —C(═O)—, —CF₂—, or a heteroatomic moiety selected from the group consisting of —O—, —S—, —NH—, and —N(C₁-C₆ alkyl)-, wherein any one or more —CH₃ groups of the C₁-C₂₀ alkyl is each optionally replaced independently with —CF₃, —CF₂H, —CH₂F, or a heteroatomic moiety selected from the group consisting of —NH(C₁-C₆ alkyl), —N(C₁-C₆ alkyl)₂, —O(C₁-C₆ alkyl), and —S(C₁-C₆ alkyl), and wherein any two heteroatomic moieties are separated from one another by at least two carbon atoms; and
B is a second therapeutic polypeptide, a therapeutic small molecule, or a second mRNA molecule having a 3′-end and a 5′-end encoding a second therapeutic polypeptide,
wherein Y is covalently attached to the 3′-end of A through a first linkage.

49. The method of claim 48, wherein the cancer is selected from the group consisting of breast cancer, lung cancer, pancreatic cancer, and colorectal cancer.

50. A method of treating, preventing, slowing the progression, or reducing the severity of cancer in a patient in need thereof, the method comprising a step of administering to the patient in need thereof a pharmaceutical formulation comprising an mRNA bioconjugate of formula (II) 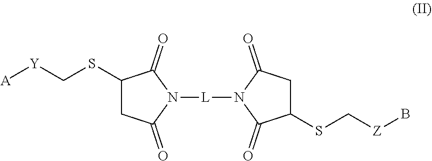
or a pharmaceutically acceptable salt thereof, and a pharmaceutically acceptable carrier, excipient, or diluent,
wherein
A is a first mRNA molecule having a 3′-end and a 5′-end, wherein A encodes a first therapeutic polypeptide;
Y is a C₁-C₂₀ alkyl, wherein any one or more —CH₂— groups of the C₁-C₂₀ alkyl is each optionally replaced independently with —C(═O)—, —CF₂—, or a heteroatomic moiety selected from the group consisting of —O—, —S—, —NH—, and —N(C₁-C₆ alkyl)-, wherein any one or more —CH₃ groups of the C₁-C₂₀ alkyl is each optionally replaced independently with —CF₃, —CF₂H, —CH₂F, or a heteroatomic moiety selected from the group consisting of —NH(C₁-C₆ alkyl), —N(C₁-C₆ alkyl)₂, —O(C₁-C₆ alkyl), and —S(C₁-C₆ alkyl), and wherein any two heteroatomic moieties are separated from one another by at least two carbon atoms;
L is a C₂-C₅₀ alkyl, C₂-C₅₀ alkenyl, C₂-C₅₀ alkynyl, C₃-C₈ cycloalkyl, aryl, heteroaryl, C₄-C₅₀ alkyl-cycloalkyl, C₇-C₅₀ alkyl-aryl, or C₆-C₅₀ alkyl-heteroaryl, wherein any one or more —CH₂— groups of the C₂-C₅₀ alkyl is each optionally replaced independently with —C(═O)—, —CF₂—, or a heteroatomic moiety selected from the group consisting of —O—, —S—, —NH—, and —N(C₁-C₆ alkyl)-, wherein any one or more —CH₃ groups of L is each optionally replaced independently with —CF₃, —CF₂H, —CH₂F, or a heteroatomic moiety selected from the group consisting of —NH(C₁-C₆ alkyl), —N(C₁-C₆ alkyl)₂, —O(C₁-C₆ alkyl), and —S(C₁-C₆ alkyl), and wherein any two heteroatomic moieties are separated from one another by at least two carbon atoms;
Z is a C₁-C₂₀ alkyl, wherein any one or more —CH₂— groups of the C₁-C₂₀ alkyl is each optionally replaced independently with —C(═O)—, —CF₂—, or a heteroatomic moiety selected from the group consisting of —O—, —S—, —NH—, and —N(C₁-C₆ alkyl)-, wherein any one or more —CH₃ groups of the C₁-C₂₀ alkyl is each optionally replaced independently with —CF₃, —CF₂H, —CH₂F, or a heteroatomic moiety selected from the group consisting of —NH(C₁-C₆ alkyl), —N(C₁-C₆ alkyl)₂, —O(C₁-C₆ alkyl), and —S(C₁-C₆ alkyl), and wherein any two heteroatomic moieties are separated from one another by at least two carbon atoms; and
B is a second therapeutic polypeptide, a therapeutic small molecule, or a second mRNA molecule having a 3′-end and a 5′-end encoding a second therapeutic polypeptide,
wherein Y is covalently attached to the 3′-end of A through a first linkage.

51. The method of claim 50, wherein the cancer is selected from the group consisting of breast cancer, lung cancer, pancreatic cancer, and colorectal cancer.

52. A method of treating, preventing, slowing the progression, or reducing the severity of obesity in a patient in need thereof, the method comprising a step of administering to the patient in need thereof a pharmaceutical formulation comprising an mRNA bioconjugate of formula (I) 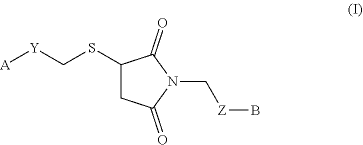
or a pharmaceutically acceptable salt thereof, and a pharmaceutically acceptable carrier, excipient, or diluent,
wherein
A is a first mRNA molecule having a 3′-end and a 5′-end, wherein A encodes a first therapeutic polypeptide;
Y is a C₁-C₂₀ alkyl, wherein any one or more —CH₂— groups of the C₁-C₂₀ alkyl is each optionally replaced independently with —C(═O)—, —CF₂—, or a heteroatomic moiety selected from the group consisting of —O—, —S—, —NH—, and —N(C₁-C₆ alkyl)-, wherein any one or more —CH₃ groups of the C₁-C₂₀ alkyl is each optionally replaced independently with —CF₃, —CF₂H, —CH₂F, or a heteroatomic moiety selected from the group consisting of —NH(C₁-C₆ alkyl), —N(C₁-C₆ alkyl)₂, —O(C₁-C₆ alkyl), and —S(C₁-C₆ alkyl), and wherein any two heteroatomic moieties are separated from one another by at least two carbon atoms;
Z is a C₁-C₂₀ alkyl, wherein any one or more —CH₂— groups of the C₁-C₂₀ alkyl is each optionally replaced independently with —C(═O)—, —CF₂—, or a heteroatomic moiety selected from the group consisting of —O—, —S—, —NH—, and —N(C₁-C₆ alkyl)-, wherein any one or more —CH₃ groups of the C₁-C₂₀ alkyl is each optionally replaced independently with —CF₃, —CF₂H, —CH₂F, or a heteroatomic moiety selected from the group consisting of —NH(C₁-C₆ alkyl), —N(C₁-C₆ alkyl)₂, —O(C₁-C₆ alkyl), and —S(C₁-C₆ alkyl), and wherein any two heteroatomic moieties are separated from one another by at least two carbon atoms; and
B is a second therapeutic polypeptide, a therapeutic small molecule, or a second mRNA molecule having a 3′-end and a 5′-end encoding a second therapeutic polypeptide,
wherein Y is covalently attached to the 3′-end of A through a first linkage.

53. A method of treating, preventing, slowing the progression, or reducing the severity of obesity in a patient in need thereof, the method comprising a step of administering to the patient in need thereof a pharmaceutical formulation comprising an mRNA bioconjugate of formula (II) 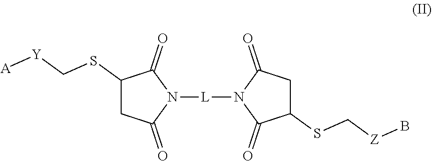
or a pharmaceutically acceptable salt thereof, and a pharmaceutically acceptable carrier, excipient, or diluent,
wherein
A is a first mRNA molecule having a 3′-end and a 5′-end, wherein A encodes a first therapeutic polypeptide;
Y is a C₁-C₂₀ alkyl, wherein any one or more —CH₂— groups of the C₁-C₂₀ alkyl is each optionally replaced independently with —C(═O)—, —CF₂—, or a heteroatomic moiety selected from the group consisting of —O—, —S—, —NH—, and —N(C₁-C₆ alkyl)-, wherein any one or more —CH₃ groups of the C₁-C₂₀ alkyl is each optionally replaced independently with —CF₃, —CF₂H, —CH₂F, or a heteroatomic moiety selected from the group consisting of —NH(C₁-C₆ alkyl), —N(C₁-C₆ alkyl)₂, —O(C₁-C₆ alkyl), and —S(C₁-C₆ alkyl), and wherein any two heteroatomic moieties are separated from one another by at least two carbon atoms;
L is a C₂-C₅₀ alkyl, C₂-C₅₀ alkenyl, C₂-C₅₀ alkynyl, C₃-C₈ cycloalkyl, aryl, heteroaryl, C₄-C₅₀ alkyl-cycloalkyl, C₇-C₅₀ alkyl-aryl, or C₆-C₅₀ alkyl-heteroaryl, wherein any one or more —CH₂— groups of the C₂-C₅₀ alkyl is each optionally replaced independently with —C(═O)—, —CF₂—, or a heteroatomic moiety selected from the group consisting of —O—, —S—, —NH—, and —N(C₁-C₆ alkyl)-, wherein any one or more —CH₃ groups of L is each optionally replaced independently with —CF₃, —CF₂H, —CH₂F, or a heteroatomic moiety selected from the group consisting of —NH(C₁-C₆ alkyl), —N(C₁-C₆ alkyl)₂, —O(C₁-C₆ alkyl), and —S(C₁-C₆ alkyl), and wherein any two heteroatomic moieties are separated from one another by at least two carbon atoms;
Z is a C₁-C₂₀ alkyl, wherein any one or more —CH₂— groups of the C₁-C₂₀ alkyl is each optionally replaced independently with —C(═O)—, —CF₂—, or a heteroatomic moiety selected from the group consisting of —O—, —S—, —NH—, and —N(C₁-C₆ alkyl)-, wherein any one or more —CH₃ groups of the C₁-C₂₀ alkyl is each optionally replaced independently with —CF₃, —CF₂H, —CH₂F, or a heteroatomic moiety selected from the group consisting of —NH(C₁-C₆ alkyl), —N(C₁-C₆ alkyl)₂, —O(C₁-C₆ alkyl), and —S(C₁-C₆ alkyl), and wherein any two heteroatomic moieties are separated from one another by at least two carbon atoms; and
B is a second therapeutic polypeptide, a therapeutic small molecule, or a second mRNA molecule having a 3′-end and a 5′-end encoding a second therapeutic polypeptide,
wherein Y is covalently attached to the 3′-end of A through a first linkage.

54. A method of enzyme replacement therapy, the method comprising a step of administering to a patient in need of enzyme replacement therapy a pharmaceutical formulation comprising an mRNA bioconjugate of formula (I) 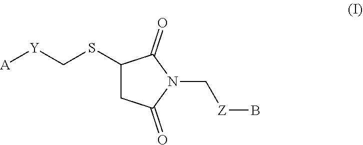
or a pharmaceutically acceptable salt thereof, and a pharmaceutically acceptable carrier, excipient, or diluent,
wherein
A is a first mRNA molecule having a 3′-end and a 5′-end, wherein A encodes a first enzyme;
Y is a C₁-C₂₀ alkyl, wherein any one or more —CH₂— groups of the C₁-C₂₀ alkyl is each optionally replaced independently with —C(═O)—, —CF₂—, or a heteroatomic moiety selected from the group consisting of —O—, —S—, —NH—, and —N(C₁-C₆ alkyl)-, wherein any one or more —CH₃ groups of the C₁-C₂₀ alkyl is each optionally replaced independently with —CF₃, —CF₂H, —CH₂F, or a heteroatomic moiety selected from the group consisting of —NH(C₁-C₆ alkyl), —N(C₁-C₆ alkyl)₂, —O(C₁-C₆ alkyl), and —S(C₁-C₆ alkyl), and wherein any two heteroatomic moieties are separated from one another by at least two carbon atoms;
Z is a C₁-C₂₀ alkyl, wherein any one or more —CH₂— groups of the C₁-C₂₀ alkyl is each optionally replaced independently with —C(═O)—, —CF₂—, or a heteroatomic moiety selected from the group consisting of —O—, —S—, —NH—, and —N(C₁-C₆ alkyl)-, wherein any one or more —CH₃ groups of the C₁-C₂₀ alkyl is each optionally replaced independently with —CF₃, —CF₂H, —CH₂F, or a heteroatomic moiety selected from the group consisting of —NH(C₁-C₆ alkyl), —N(C₁-C₆ alkyl)₂, —O(C₁-C₆ alkyl), and —S(C₁-C₆ alkyl), and wherein any two heteroatomic moieties are separated from one another by at least two carbon atoms; and
B is an oligonucleotide, a polypeptide, a protein, a second enzyme, a small molecule, a carbohydrate, a lipid, a polyethylene glycol (PEG) molecule, or a biopolymer,
wherein Y is covalently attached to the 3′-end of A through a first linkage, and wherein the patient in need of enzyme replacement therapy is deficient of the first enzyme.

55. The method of claim 54, wherein B is a second enzyme, and wherein the patient in need of enzyme replacement therapy is deficient of the second enzyme.

56. The method of claim 55, wherein the patient in need of enzyme replacement therapy is no longer deficient of the first enzyme, the second enzyme, or both after the administering.

57. A method of enzyme replacement therapy, the method comprising a step of administering to a patient in need of enzyme replacement therapy a pharmaceutical formulation comprising an mRNA bioconjugate of formula (II) 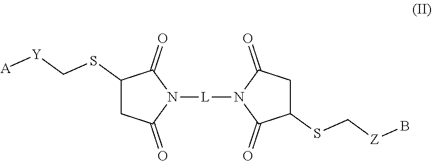
or a pharmaceutically acceptable salt thereof, and a pharmaceutically acceptable carrier, excipient, or diluent,
wherein
A is a first mRNA molecule having a 3′-end and a 5′-end, wherein A encodes a first enzyme;
Y is a C₁-C₂₀ alkyl, wherein any one or more —CH₂— groups of the C₁-C₂₀ alkyl is each optionally replaced independently with —C(═O)—, —CF₂—, or a heteroatomic moiety selected from the group consisting of —O—, —S—, —NH—, and —N(C₁-C₆ alkyl)-, wherein any one or more —CH₃ groups of the C₁-C₂₀ alkyl is each optionally replaced independently with —CF₃, —CF₂H, —CH₂F, or a heteroatomic moiety selected from the group consisting of —NH(C₁-C₆ alkyl), —N(C₁-C₆ alkyl)₂, —O(C₁-C₆ alkyl), and —S(C₁-C₆ alkyl), and wherein any two heteroatomic moieties are separated from one another by at least two carbon atoms;
L is a C₂-C₅₀ alkyl, C₂-C₅₀ alkenyl, C₂-C₅₀ alkynyl, C₃-C₈ cycloalkyl, aryl, heteroaryl, C₄-C₅₀ alkyl-cycloalkyl, C₇-C₅₀ alkyl-aryl, or C₆-C₅₀ alkyl-heteroaryl, wherein any one or more —CH₂— groups of the C₂-C₅₀ alkyl is each optionally replaced independently with —C(═O)—, —CF₂—, or a heteroatomic moiety selected from the group consisting of —O—, —S—, —NH—, and —N(C₁-C₆ alkyl)-, wherein any one or more —CH₃ groups of L is each optionally replaced independently with —CF₃, —CF₂H, —CH₂F, or a heteroatomic moiety selected from the group consisting of —NH(C₁-C₆ alkyl), —N(C₁-C₆ alkyl)₂, —O(C₁-C₆ alkyl), and —S(C₁-C₆ alkyl), and wherein any two heteroatomic moieties are separated from one another by at least two carbon atoms;
Z is a C₁-C₂₀ alkyl, wherein any one or more —CH₂— groups of the C₁-C₂₀ alkyl is each optionally replaced independently with —C(═O)—, —CF₂—, or a heteroatomic moiety selected from the group consisting of —O—, —S—, —NH—, and —N(C₁-C₆ alkyl)-, wherein any one or more —CH₃ groups of the C₁-C₂₀ alkyl is each optionally replaced independently with —CF₃, —CF₂H, —CH₂F, or a heteroatomic moiety selected from the group consisting of —NH(C₁-C₆ alkyl), —N(C₁-C₆ alkyl)₂, —O(C₁-C₆ alkyl), and —S(C₁-C₆ alkyl), and wherein any two heteroatomic moieties are separated from one another by at least two carbon atoms; and
B is an oligonucleotide, a polypeptide, a protein, a second enzyme, a small molecule, a carbohydrate, a lipid, a polyethylene glycol (PEG) molecule, or a biopolymer,
wherein Y is covalently attached to the 3′-end of A through a first linkage, and wherein the patient in need of enzyme replacement therapy is deficient of the first enzyme.

58. The method of claim 57, wherein B is a second enzyme, and wherein the patient in need of enzyme replacement therapy is deficient of the second enzyme.

59. The method of claim 58, wherein the patient in need of enzyme replacement therapy is no longer deficient of the first enzyme, the second enzyme, or both after the administering.


SEQUENCE LISTING (129 sequences)

SEQ ID NO: 1 - protein, 9 aa, synthetic construct
  REGION 1..9: note = Description of sequence: artificial peptide
RKKRRQRRR

SEQ ID NO: 2 - protein, 16 aa, synthetic construct
  REGION 1..16: note = Description of sequence: artificial peptide
RQIKIWFQNRRMKWKK

SEQ ID NO: 3 - protein, 8 aa, synthetic construct
  REGION 1..8: note = Description of sequence: artificial peptide
RRRRRRRR

SEQ ID NO: 4 - protein, 7 aa, synthetic construct
  REGION 1..7: note = Description of sequence: artificial peptide
  SITE 2: note = X is l-2-naphthylalanine
  REGION 1..7: note = cyclic peptide
  SITE 1..7: note = phenylalanine-glutamate bond
FXRRRRQ

SEQ ID NO: 5 - protein, 19 aa, synthetic construct
  REGION 1..19: note = Description of sequence: artificial peptide
KETWWETWWTEWSQPKKRK

SEQ ID NO: 6 - protein, 26 aa, synthetic construct
  REGION 1..26: note = Description of sequence: artificial peptide
GLAFLGFLGAAGSTMGAWSQPKKKRK

SEQ ID NO: 7 - protein, 21 aa, synthetic construct
  REGION 1..21: note = Description of sequence: artificial peptide
MVRRFLVTLRIRRACGPPRVR

SEQ ID NO: 8 - protein, 30 aa, synthetic construct
  REGION 1..30: note = Description of sequence: artificial peptide
MVKSKIGSWILVLFVAMWSDVGLCKKRPKP

SEQ ID NO: 9 - protein, 17 aa, synthetic construct
  REGION 1..17: note = Description of sequence: artificial peptide
KLALKALKALKAALKLA

SEQ ID NO: 10 - protein, 27 aa, synthetic construct
  REGION 1..27: note = Description of sequence: artificial peptide
GWTLNSAGYLLGKINLKALAALAKKIL

SEQ ID NO: 11 - protein, 21 aa, synthetic construct
  REGION 1..21: note = Description of sequence: artificial peptide
AGYLLGKINLKALAALAKKIL

SEQ ID NO: 12 - protein, 20 aa, synthetic construct
  REGION 1..20: note = Description of sequence: artificial peptide
GLWRALWRLLRSLWRLLWRA

SEQ ID NO: 13 - protein, 19 aa, synthetic construct
  REGION 1..19: note = Description of sequence: artificial peptide
KWLLRWLSRLLRWLARWLG

SEQ ID NO: 14 - protein, 36 aa, synthetic construct
  REGION 1..36: note = Description of sequence: artificial peptide
GLFEAIEGFIENGWEGMIDGWYGGGGRRRRRRRRRK

SEQ ID NO: 15 - protein, 27 aa, synthetic construct
  REGION 1..27: note = Description of sequence: artificial peptide
LSTAADMQGVVTDGMASGLDKDYLKPD

SEQ ID NO: 16 - protein, 21 aa, synthetic construct
  REGION 1..21: note = Description of sequence: artificial peptide
RRIRPRPPRLPRPRPRPLPFP

SEQ ID NO: 17 - protein, 26 aa, synthetic construct
  REGION 1..26: note = Description of sequence: artificial peptide
GIGAVLKVLTTGLPALISWIKRKRQQ

SEQ ID NO: 18 - protein, 17 aa, synthetic construct
  REGION 1..17: note = Description of sequence: artificial peptide
CSIPPEVKFNKPFVYLI

SEQ ID NO: 19 - protein, 14 aa, synthetic construct
  REGION 1..14: note = Description of sequence: artificial peptide
SDLWEMMMVSLACQ

SEQ ID NO: 20 - protein, 7 aa, synthetic construct
  REGION 1..7: note = Description of sequence: artificial peptide
LGAQSNF

SEQ ID NO: 21 - protein, 12 aa, synthetic construct
  REGION 1..12: note = Description of sequence: artificial peptide
PLILLRLLRGQF

SEQ ID NO: 22 - protein, 6 aa, synthetic construct
  REGION 1..6: note = Description of sequence: artificial peptide
PFVYLI

SEQ ID NO: 23 - protein, 8 aa, synthetic construct
  REGION 1..8: note = Description of sequence: artificial peptide
WSYGLRPG

SEQ ID NO: 24 - protein, 30 aa, synthetic construct
  REGION 1..30: note = Description of sequence: artificial peptide
WEARLARALARALARHLARALARALRACEA

SEQ ID NO: 25 - protein, 21 aa, synthetic construct
  REGION 1..21: note = Description of sequence: artificial peptide
  MOD_RES 1: note = Stearyl-Alanine
AGYLLGKLLOOLAAAALOOLL

SEQ ID NO: 26 - protein, 27 aa, synthetic construct
  REGION 1..27: note = Description of sequence: artificial peptide
WEAKLAKALAKALAKHLAKALAKALKA

SEQ ID NO: 27 - protein, 30 aa, synthetic construct
  REGION 1..30: note = Description of sequence: artificial peptide
WEAALAEALAEALAEHLAEALAEALEALAA

SEQ ID NO: 28 - protein, 24 aa, synthetic construct
  REGION 1..24: note = Description of sequence: artificial peptide
IGKEFKRIVERIKRFLRELVRPLR

SEQ ID NO: 29 - protein, 26 aa, synthetic construct
  REGION 1..26: note = Description of sequence: artificial peptide
KKALLAHALHLLALLALHLAHALKKA

SEQ ID NO: 30 - protein, 26 aa, synthetic construct
  REGION 1..26: note = Description of sequence: artificial peptide
KKALLALALHHLAHLALHLALALKKA

SEQ ID NO: 31 - protein, 21 aa, synthetic construct
  REGION 1..21: note = Description of sequence: artificial peptide
KLLLLKLLLLKLLLLKLLLLK

SEQ ID NO: 32 - protein, 15 aa, synthetic construct
  REGION 1..15: note = Description of sequence: artificial peptide
  MOD_RES 3: note = Aib
  MOD_RES 6: note = Aib
  MOD_RES 9: note = Aib
  MOD_RES 12: note = Aib
  MOD_RES 15: note = Aib
RRARRARRARRARRA

SEQ ID NO: 33 - protein, 9 aa, synthetic construct
  REGION 1..9: note = Description of sequence: artificial peptide
RRRRRRRRR

SEQ ID NO: 34 - protein, 23 aa, synthetic construct
  REGION 1..23: note = Description of sequence: artificial peptide
MAPFASLASGILLLLSLITSSKA

SEQ ID NO: 35 - protein, 29 aa, synthetic construct
  REGION 1..29: note = Description of sequence: artificial peptide
MLLGPGHTLSAPALALAVTLTLLVRSASP

SEQ ID NO: 36 - protein, 17 aa, synthetic construct
  REGION 1..17: note = Description of sequence: artificial peptide
MLLSVPLLLGLLGLAAA

SEQ ID NO: 37 - protein, 21 aa, synthetic construct
  REGION 1..21: note = Description of sequence: artificial peptide
MQELRGILLCLLLAAAVPTTP

SEQ ID NO: 38 - protein, 16 aa, synthetic construct
  REGION 1..16: note = Description of sequence: artificial peptide
MRYVASYLLAALGGNS

SEQ ID NO: 39 - protein, 21 aa, synthetic construct
  REGION 1..21: note = Description of sequence: artificial peptide
MGKSPEAWCIVLFSVLASFSA

SEQ ID NO: 40 - protein, 25 aa, synthetic construct
  REGION 1..25: note = Description of sequence: artificial peptide
MASSGSVQQPRLVLLMLVLAGAARA

SEQ ID NO: 41 - protein, 24 aa, synthetic construct
  REGION 1..24: note = Description of sequence: artificial peptide
MRWKIIQLQYCFLLVPCMLTALEA

SEQ ID NO: 42 - protein, 19 aa, synthetic construct
  REGION 1..19: note = Description of sequence: artificial peptide
MLSRSLLCLALAWVARVGA

SEQ ID NO: 43 - protein, 24 aa, synthetic construct
  REGION 1..24: note = Description of sequence: artificial peptide
MRFSCLALLPGVALLLASARLAAA

SEQ ID NO: 44 - protein, 21 aa, synthetic construct
  REGION 1..21: note = Description of sequence: artificial peptide
MRVLWVLGLCCVLLTFGFVRA

SEQ ID NO: 45 - protein, 18 aa, synthetic construct
  REGION 1..18: note = Description of sequence: artificial peptide
MKFPMVAAALLLLCAVRA

SEQ ID NO: 46 - protein, 17 aa, synthetic construct
  REGION 1..17: note = Description of sequence: artificial peptide
MRSLLLASFCLLAVALA

SEQ ID NO: 47 - protein, 21 aa, synthetic construct
  REGION 1..21: note = Description of sequence: artificial peptide
MKILLLCVGLLLTWDNGMVLG

SEQ ID NO: 48 - protein, 33 aa, synthetic construct
  REGION 1..33: note = Description of sequence: artificial peptide
MLRISGRNMKVLFAAALIVGSVVFLLLPGPSVA

SEQ ID NO: 49 - protein, 30 aa, synthetic construct
  REGION 1..30: note = Description of sequence: artificial peptide
MAATVRRQRPRRLLCWTLVAVLLADLLALS

SEQ ID NO: 50 - protein, 27 aa, synthetic construct
  REGION 1..27: note = Description of sequence: artificial peptide
MKMGVRLAARAWPLCGLLLAALGGVCA

SEQ ID NO: 51 - protein, 23 aa, synthetic construct
  REGION 1..23: note = Description of sequence: artificial peptide
MWWRLWWLLLLLLLLWLALAAAA

SEQ ID NO: 52 - protein, 19 aa, synthetic construct
  REGION 1..19: note = Description of sequence: artificial peptide
MGWSLILLFLVAVATRVLS

SEQ ID NO: 53 - protein, 22 aa, synthetic construct
  REGION 1..22: note = Description of sequence: artificial peptide
MDFQVQIISFLLISASVIMSRG

SEQ ID NO: 54 - protein, 19 aa, synthetic construct
  REGION 1..19: note = Description of sequence: artificial peptide
MEFGLSWVFLVALFRGVQC

SEQ ID NO: 55 - protein, 18 aa, synthetic construct
  REGION 1..18: note = Description of sequence: artificial peptide
MKWVTFISLLFLFSSAYS

SEQ ID NO: 56 - protein, 19 aa, synthetic construct
  REGION 1..19: note = Description of sequence: artificial peptide
MKLPVRLLVLMFWIPAASA

SEQ ID NO: 57 - protein, 15 aa, synthetic construct
  REGION 1..15: note = Description of sequence: artificial peptide
MNLLLILTFVAAAVA

SEQ ID NO: 58 - protein, 20 aa, synthetic construct
  REGION 1..20: note = Description of sequence: artificial peptide
MGSAALLLWVLLLWVPSSRA

SEQ ID NO: 59 - protein, 19 aa, synthetic construct
  REGION 1..19: note = Description of sequence: artificial peptide
MTRLTVLALLAGLLASSRA

SEQ ID NO: 60 - protein, 23 aa, synthetic construct
  REGION 1..23: note = Description of sequence: artificial peptide
MWWRLWWLLLLLLLLWPMVWAAA

SEQ ID NO: 61 - protein, 19 aa, synthetic construct
  REGION 1..19: note = Description of sequence: artificial peptide
MKLPVRLLVLMFWIPASSS

SEQ ID NO: 62 - protein, 22 aa, synthetic construct
  REGION 1..22: note = Description of sequence: artificial peptide
MDMRVPAQLLGLLLLWLSGARC

SEQ ID NO: 63 - protein, 22 aa, synthetic construct
  REGION 1..22: note = Description of sequence: artificial peptide
MKYLLPTAAAGLLLLAAQPAMA

SEQ ID NO: 64 - protein, 17 aa, synthetic construct
  REGION 1..17: note = Description of sequence: artificial peptide
MGVKVLFALICIAVAEA

SEQ ID NO: 65 - protein, 16 aa, synthetic construct
  REGION 1..16: note = Description of sequence: artificial peptide
MPLLLLLPLLWAGALA

SEQ ID NO: 66 - protein, 22 aa, synthetic construct
  REGION 1..22: note = Description of sequence: artificial peptide
MRARALLAVLLLLLLVGIAAAA

SEQ ID NO: 67 - protein, 22 aa, synthetic construct
  REGION 1..22: note = Description of sequence: artificial peptide
MATATLLAVLLLLLLVGSAGGA

SEQ ID NO: 68 - protein, 22 aa, synthetic construct
  REGION 1..22: note = Description of sequence: artificial peptide
MRARALLVVLVLVVLLGVASSA

SEQ ID NO: 69 - protein, 22 aa, synthetic construct
  REGION 1..22: note = Description of sequence: artificial peptide
MPGPGAALLLLLLVLLGLGSAA

SEQ ID NO: 70 - protein, 22 aa, synthetic construct
  REGION 1..22: note = Description of sequence: artificial peptide
MTTTTVLLLLVLVVLAGLTSGA

SEQ ID NO: 71 - protein, 15 aa, synthetic construct
  REGION 1..15: note = Description of sequence: artificial peptide
QFGDFDPSVEEEEDL

SEQ ID NO: 72 - RNA, 720 nt, synthetic construct
  misc_feature 1..720: note = Description of sequence: artificial sequence
  source 1..720: mol_type = other RNA
atggtgagcaagggcgaggaggacaacatggccatcatcaaggagttcatgcggttcaaggtgcacatggagggcagcgtgaacggccacgagttcgagatcgagggcgagggcgagggccggccctacgagggcacccagaccgccaagctgaaggtgaccaagggcggccccctgcccttcgcctgggacatcctgagcccccagttcatgtacggcagcaaggcctacgtgaagcaccccgccgacatccccgactacctgaagctgagcttccccgagggcttcaagtgggagcgggtgatgaacttcgaggacggcggcgtggtgaccgtgacccaggacagcagcctgcaggacggcgagttcatctacaaggtgaagctgcggggcaccaacttccccagcgacggccccgtgatgcagaagaagaccatgggctgggaggccagcagcgagcggatgtaccccgaggacggcgccctgaagggcgagatcaagcagcggctgaagctgaaggacggcggccactacgacgccgaggtgaagaccacctacaaggccaagaagcccgtgcagctgcccggcgcctacaacgtgaacatcaagctggacatcaccagccacaacgaggactacaccatcgtggagcagtacgagcgggccgagggccggcacagcaccggcggcatggacgagctgtacaagagcggcaactga

SEQ ID NO: 73 - RNA, 720 nt, synthetic construct
  misc_feature 1..720: note = Description of sequence: artificial sequence
  source 1..720: mol_type = other RNA
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 74 - RNA, 1653 nt, synthetic construct
  misc_feature 1..1653: note = Description of sequence: artificial sequence
  source 1..1653: mol_type = other RNA
atggaggacgccaagaacatcaagaagggccccgcccccttctaccccctggaggacggcaccgccggcgagcagctgcacaaggccatgaagcggtacgccctggtgcccggcaccatcgccttcaccgacgcccacatcgaggtggacatcacctacgccgagtacttcgagatgagcgtgcggctggccgaggccatgaagcggtacggcctgaacaccaaccaccggatcgtggtgtgcagcgagaacagcctgcagttcttcatgcccgtgctgggcgccctgttcatcggcgtggccgtggcccccgccaacgacatctacaacgagcgggagctgctgaacagcatgggcatcagccagcccaccgtggtgttcgtgagcaagaagggcctgcagaagatcctgaacgtgcagaagaagctgcccatcatccagaagatcatcatcatggacagcaagaccgactaccagggcttccagagcatgtacaccttcgtgaccagccacctgccccccggcttcaacgagtacgacttcgtgcccgagagcttcgaccgggacaagaccatcgccctgatcatgaacagcagcggcagcaccggcctgcccaagggcgtggccctgccccaccggaccgcctgcgtgcggttcagccacgcccgggaccccatcttcggcaaccagatcatccccgacaccgccatcctgagcgtggtgcccttccaccacggcttcggcatgttcaccaccctgggctacctgatctgcggcttccgggtggtgctgatgtaccggttcgaggaggagctgttcctgcggagcctgcaggactacaagatccagagcgccctgctggtgcccaccctgttcagcttcttcgccaagagcaccctgatcgacaagtacgacctgagcaacctgcacgagatcgccagcggcggcgcccccctgagcaaggaggtgggcgaggccgtggccaagcggttccacctgcccggcatccggcagggctacggcctgaccgagaccaccagcgccatcctgatcacccccgagggcgacgacaagcccggcgccgtgggcaaggtggtgcccttcttcgaggccaaggtggtggacctggacaccggcaagaccctgggcgtgaaccagcggggcgagctgtgcgtgcggggccccatgatcatgagcggctacgtgaacaaccccgaggccaccaacgccctgatcgacaaggacggctggctgcacagcggcgacatcgcctactgggacgaggacgagcacttcttcatcgtggaccggctgaagagcctgatcaagtacaagggctaccaggtggcccccgccgagctggagagcatcctgctgcagcaccccaacatcttcgacgccggcgtggccggcctgcccgacgacgacgccggcgagctgcccgccgccgtggtggtgctggagcacggcaagaccatgaccgagaaggagatcgtggactacgtggccagccaggtgaccaccgccaagaagctgcggggcggcgtggtgttcgtggacgaggtgcccaagggcctgaccggcaagctggacgcccggaagatccgggagatcctgatcaaggccaagaagggcggcaagatcgccgtgtga

SEQ ID NO: 75 - DNA, 17 bp, synthetic construct
  misc_feature 1..17: note = Description of sequence: construct, name: PolyA-gamma-mercaptopropanol
  source 1..17: mol_type = other DNA
  modified_base 1: mod_base = OTHER; note = 5'phosphorylation
  modified_base 17: mod_base = OTHER; note = deoxyadenosine-1-O-dimethoxytrityl-propyl-disulfide -propanol
  misc_feature 1..16: note = RNA
  misc_feature 17: note = DNA
  misc_feature 1..17: note = molecule is DNA/RNA hybrid (every T represent uracil)
aaaaaaaaaaaaaaaaa

SEQ ID NO: 76 - DNA, 17 bp, synthetic construct
  misc_feature 1..17: note = Description of sequence: construct, name: PolyA-SH
  source 1..17: mol_type = other DNA
  modified_base 1: mod_base = OTHER; note = 5'phosphorylation
  modified_base 17: mod_base = OTHER; note = deoxyadenosine-1-O-dimethoxytrityl-propyl-disulfide
  misc_feature 1..16: note = RNA
  misc_feature 17: note = DNA
  misc_feature 1..17: note = molecule is DNA/RNA hybrid (every T represent uracil)
aaaaaaaaaaaaaaaaa

SEQ ID NO: 77 - RNA, 16 nt, synthetic construct
  misc_feature 1..16: note = Description of sequence: construct, name: Non-Canonical PolyA-gamma-mercaptopropanol
  source 1..16: mol_type = other RNA
  modified_base 1: mod_base = OTHER; note = 5'phosphorylation
  modified_base 11: mod_base = OTHER; note = 2'-O-methoxy-ethyl adenosine
  misc_feature 11^12: note = 5'-3'-phosphorothioate linkage
  modified_base 12: mod_base = OTHER; note = 2'-O-methoxy-ethyl adenosine
  misc_feature 12^13: note = 5'-3'-phosphorothioate linkage
  modified_base 13: mod_base = OTHER; note = 2'-O-methoxy-ethyl adenosine
  misc_feature 13^14: note = 5'-3'-phosphorothioate linkage
  modified_base 14: mod_base = OTHER; note = 2'-O-methoxy-ethyl adenosine
  misc_feature 14^15: note = 5'-3'-phosphorothioate linkage
  modified_base 15: mod_base = OTHER; note = 2'-O-methoxy-ethyl adenosine
  misc_feature 15^16: note = 5'-3'-phosphorothioate linkage
  modified_base 16: mod_base = OTHER; note = 2'-O-methoxy-ethyl adenosine-phosphorothioate-1-O-d imethoxytrityl-propyl-disulfide-propanol
aaaaaaaaaaaaaaaa

SEQ ID NO: 78 - DNA, 17 bp, synthetic construct
  misc_feature 1..17: note = Description of sequence: construct, name: Non-Canonical PolyA-dideoxynucleotide
  source 1..17: mol_type = other DNA
  modified_base 1: mod_base = OTHER; note = 5'phosphorylation
  modified_base 11: mod_base = OTHER; note = 2'-O-methoxy-ethyl adenosine
  misc_feature 11^12: note = 5'-3'-phosphorothioate linkage
  modified_base 12: mod_base = OTHER; note = 2'-O-methoxy-ethyl adenosine
  misc_feature 12^13: note = 5'-3'-phosphorothioate linkage
  modified_base 13: mod_base = OTHER; note = 2'-O-methoxy-ethyl adenosine
  misc_feature 13^14: note = 5'-3'-phosphorothioate linkage
  modified_base 14: mod_base = OTHER; note = 2'-O-methoxy-ethyl adenosine
  misc_feature 14^15: note = 5'-3'-phosphorothioate linkage
  modified_base 15: mod_base = OTHER; note = 2'-O-methoxy-ethyl adenosine
  misc_feature 15^16: note = 5'-3'-phosphorothioate linkage
  modified_base 16: mod_base = OTHER; note = 2'-O-methoxy-ethyl adenosine
  misc_feature 15^16: note = 5'-3'-phosphorothioate linkage
  modified_base 17: mod_base = OTHER; note = dideoxycytosine
  misc_feature 1..16: note = RNA
  misc_feature 17: note = DNA
  misc_feature 1..17: note = molecule is DNA/RNA hybrid (every T represent uracil)
aaaaaaaaaaaaaaaac

SEQ ID NO: 79 - DNA, 17 bp, synthetic construct
  misc_feature 1..17: note = Description of sequence: construct, name: PolyA-S-Mal-SulfoCy5
  source 1..17: mol_type = other DNA
  modified_base 1: mod_base = OTHER; note = 5'phosphorylation
  modified_base 17: mod_base = OTHER; note = deoxyadenosine-3'methoxypropanethiol-maleimide-3H-I ndolium, 1-[6-[(6-aminohexyl)amino]-6-oxohexyl]-2-[5-(1,3- dihydro-1,3,3-trimethyl-5-sulfo-2H-indol-2-ylidene)-1,3-pe ntadien-1-yl]-3,3-dimethyl-5-sulfo-[salt]
  misc_feature 1..16: note = RNA
  misc_feature 17: note = DNA
  misc_feature 1..17: note = molecule is DNA/RNA hybrid (every T represent uracil)
aaaaaaaaaaaaaaaaa

SEQ ID NO: 80 - DNA, 17 bp, synthetic construct
  misc_feature 1..17: note = Description of sequence: construct, name: PolyA-S-Mal-P19-Mal
  source 1..17: mol_type = other DNA
  modified_base 1: mod_base = OTHER; note = 5'phosphorylation
  modified_base 17: mod_base = OTHER; note = deoxyadenosine-3'methoxypropanethiol-maleimide-nove mdec-ethylether-maleimide
aaaaaaaaaaaaaaaaa

SEQ ID NO: 81 - DNA, 17 bp, synthetic construct
  misc_feature 1..17: note = Description of sequence: construct, name: PolyA-S-Mal-P19-Mal-S-PolyA
  source 1..17: mol_type = other DNA
  modified_base 1: mod_base = OTHER; note = 5'phosphorylation
  modified_base 17: mod_base = OTHER; note = deoxyadenosine-3'methoxpropanethiol-maleimide-novem dec-ethylether-maleimide-polyA-linkage
aaaaaaaaaaaaaaaaa

SEQ ID NO: 82 - DNA, 1931 bp, synthetic construct
  misc_feature 1..1931: note = Description of sequence: construct, name: FLuc-NC-ddnt
  source 1..1931: mol_type = other DNA
  modified_base 1925: mod_base = OTHER; note = 2'-O-methoxy-ethyl adenosine
  misc_feature 1925^1926: note = 5'-3'-phosphorothioate linkage
  modified_base 1926: mod_base = OTHER; note = 2'-O-methoxy-ethyl adenosine
  misc_feature 1926^1927: note = 5'-3'-phosphorothioate linkage
  modified_base 1927: mod_base = OTHER; note = 2'-O-methoxy-ethyl adenosine
  misc_feature 1927^1928: note = 5'-3'-phosphorothioate linkage
  modified_base 1928: mod_base = OTHER; note = 2'-O-methoxy-ethyl adenosine
  misc_feature 1928^1929: note = 5'-3'-phosphorothioate linkage
  modified_base 1929: mod_base = OTHER; note = 2'-O-methoxy-ethyl adenosine
  misc_feature 1929^1930: note = 5'-3'-phosphorothioate linkage
  modified_base 1930: mod_base = OTHER; note = 2'-O-methoxy-ethyl adenosine
  misc_feature 1930^1931: note = 5'-3'-phosphorothioate linkage
  modified_base 1931: mod_base = OTHER; note = dideoxycytosine
  misc_feature 1..1930: note = RNA
  misc_feature 1931: note = DNA
  misc_feature 1..1931: note = molecule is DNA/RNA hybrid (every T represent uracil)
aggaaataagagagaaaagaagagtaagaagaaatataagagccaccatggaggacgccaagaacatcaagaagggccccgcccccttctaccccctggaggacggcaccgccggcgagcagctgcacaaggccatgaagcggtacgccctggtgcccggcaccatcgccttcaccgacgcccacatcgaggtggacatcacctacgccgagtacttcgagatgagcgtgcggctggccgaggccatgaagcggtacggcctgaacaccaaccaccggatcgtggtgtgcagcgagaacagcctgcagttcttcatgcccgtgctgggcgccctgttcatcggcgtggccgtggcccccgccaacgacatctacaacgagcgggagctgctgaacagcatgggcatcagccagcccaccgtggtgttcgtgagcaagaagggcctgcagaagatcctgaacgtgcagaagaagctgcccatcatccagaagatcatcatcatggacagcaagaccgactaccagggcttccagagcatgtacaccttcgtgaccagccacctgccccccggcttcaacgagtacgacttcgtgcccgagagcttcgaccgggacaagaccatcgccctgatcatgaacagcagcggcagcaccggcctgcccaagggcgtggccctgccccaccggaccgcctgcgtgcggttcagccacgcccgggaccccatcttcggcaaccagatcatccccgacaccgccatcctgagcgtggtgcccttccaccacggcttcggcatgttcaccaccctgggctacctgatctgcggcttccgggtggtgctgatgtaccggttcgaggaggagctgttcctgcggagcctgcaggactacaagatccagagcgccctgctggtgcccaccctgttcagcttcttcgccaagagcaccctgatcgacaagtacgacctgagcaacctgcacgagatcgccagcggcggcgcccccctgagcaaggaggtgggcgaggccgtggccaagcggttccacctgcccggcatccggcagggctacggcctgaccgagaccaccagcgccatcctgatcacccccgagggcgacgacaagcccggcgccgtgggcaaggtggtgcccttcttcgaggccaaggtggtggacctggacaccggcaagaccctgggcgtgaaccagcggggcgagctgtgcgtgcggggccccatgatcatgagcggctacgtgaacaaccccgaggccaccaacgccctgatcgacaaggacggctggctgcacagcggcgacatcgcctactgggacgaggacgagcacttcttcatcgtggaccggctgaagagcctgatcaagtacaagggctaccaggtggcccccgccgagctggagagcatcctgctgcagcaccccaacatcttcgacgccggcgtggccggcctgcccgacgacgacgccggcgagctgcccgccgccgtggtggtgctggagcacggcaagaccatgaccgagaaggagatcgtggactacgtggccagccaggtgaccaccgccaagaagctgcggggcggcgtggtgttcgtggacgaggtgcccaagggcctgaccggcaagctggacgcccggaagatccgggagatcctgatcaaggccaagaagggcggcaagatcgccgtgtaagctgccttctgcggggcttgccttctggccatgcccttcttctctcccttgcacctgtacctcttggtctttgaataaagcctgagtaggaagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaac

SEQ ID NO: 83 - RNA, 1930 nt, synthetic construct
  misc_feature 1..1930: note = Description of sequence: construct, name: FLuc-NC-SR
  source 1..1930: mol_type = other RNA
  modified_base 1925: mod_base = OTHER; note = 2'-O-methoxy-ethyl adenosine
  misc_feature 1925^1926: note = 5'-3'-phosphorothioate linkage
  modified_base 1926: mod_base = OTHER; note = 2'-O-methoxy-ethyl adenosine
  misc_feature 1926^1927: note = 5'-3'-phosphorothioate linkage
  modified_base 1927: mod_base = OTHER; note = 2'-O-methoxy-ethyl adenosine
  misc_feature 1927^1928: note = 5'-3'-phosphorothioate linkage
  modified_base 1928: mod_base = OTHER; note = 2'-O-methoxy-ethyl adenosine
  misc_feature 1928^1929: note = 5'-3'-phosphorothioate linkage
  modified_base 1929: mod_base = OTHER; note = 2'-O-methoxy-ethyl adenosine
  misc_feature 1929^1930: note = 5'-3'-phosphorothioate linkage
  modified_base 1930: mod_base = OTHER; note = 2'-O-methoxy-ethyl adenosine-phosphorothioate-1-O-d imethoxytrityl-propyl-disulfide-propanol
aggaaataagagagaaaagaagagtaagaagaaatataagagccaccatggaggacgccaagaacatcaagaagggccccgcccccttctaccccctggaggacggcaccgccggcgagcagctgcacaaggccatgaagcggtacgccctggtgcccggcaccatcgccttcaccgacgcccacatcgaggtggacatcacctacgccgagtacttcgagatgagcgtgcggctggccgaggccatgaagcggtacggcctgaacaccaaccaccggatcgtggtgtgcagcgagaacagcctgcagttcttcatgcccgtgctgggcgccctgttcatcggcgtggccgtggcccccgccaacgacatctacaacgagcgggagctgctgaacagcatgggcatcagccagcccaccgtggtgttcgtgagcaagaagggcctgcagaagatcctgaacgtgcagaagaagctgcccatcatccagaagatcatcatcatggacagcaagaccgactaccagggcttccagagcatgtacaccttcgtgaccagccacctgccccccggcttcaacgagtacgacttcgtgcccgagagcttcgaccgggacaagaccatcgccctgatcatgaacagcagcggcagcaccggcctgcccaagggcgtggccctgccccaccggaccgcctgcgtgcggttcagccacgcccgggaccccatcttcggcaaccagatcatccccgacaccgccatcctgagcgtggtgcccttccaccacggcttcggcatgttcaccaccctgggctacctgatctgcggcttccgggtggtgctgatgtaccggttcgaggaggagctgttcctgcggagcctgcaggactacaagatccagagcgccctgctggtgcccaccctgttcagcttcttcgccaagagcaccctgatcgacaagtacgacctgagcaacctgcacgagatcgccagcggcggcgcccccctgagcaaggaggtgggcgaggccgtggccaagcggttccacctgcccggcatccggcagggctacggcctgaccgagaccaccagcgccatcctgatcacccccgagggcgacgacaagcccggcgccgtgggcaaggtggtgcccttcttcgaggccaaggtggtggacctggacaccggcaagaccctgggcgtgaaccagcggggcgagctgtgcgtgcggggccccatgatcatgagcggctacgtgaacaaccccgaggccaccaacgccctgatcgacaaggacggctggctgcacagcggcgacatcgcctactgggacgaggacgagcacttcttcatcgtggaccggctgaagagcctgatcaagtacaagggctaccaggtggcccccgccgagctggagagcatcctgctgcagcaccccaacatcttcgacgccggcgtggccggcctgcccgacgacgacgccggcgagctgcccgccgccgtggtggtgctggagcacggcaagaccatgaccgagaaggagatcgtggactacgtggccagccaggtgaccaccgccaagaagctgcggggcggcgtggtgttcgtggacgaggtgcccaagggcctgaccggcaagctggacgcccggaagatccgggagatcctgatcaaggccaagaagggcggcaagatcgccgtgtaagctgccttctgcggggcttgccttctggccatgcccttcttctctcccttgcacctgtacctcttggtctttgaataaagcctgagtaggaagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 84 - RNA, 1930 nt, synthetic construct; identical to SEQ ID NO: 83
  misc_feature 1..1930: note = Description of sequence: construct, name: FLuc-NC-SH
  source 1..1930: mol_type = other RNA
  modified_base 1925: mod_base = OTHER; note = 2'-O-methoxy-ethyl adenosine
  misc_feature 1925^1926: note = 5'-3'-phosphorothioate linkage
  modified_base 1926: mod_base = OTHER; note = 2'-O-methoxy-ethyl adenosine
  misc_feature 1926^1927: note = 5'-3'-phosphorothioate linkage
  modified_base 1927: mod_base = OTHER; note = 2'-O-methoxy-ethyl adenosine
  misc_feature 1927^1928: note = 5'-3'-phosphorothioate linkage
  modified_base 1928: mod_base = OTHER; note = 2'-O-methoxy-ethyl adenosine
  misc_feature 1928^1929: note = 5'-3'-phosphorothioate linkage
  modified_base 1929: mod_base = OTHER; note = 2'-O-methoxy-ethyl adenosine
  misc_feature 1929^1930: note = 5'-3'-phosphorothioate linkage
  modified_base 1930: mod_base = OTHER; note = deoxyadenosine-1-O-dimethoxytrityl-propyl-disulfide

SEQ ID NO: 85 - DNA, 998 bp, synthetic construct
  misc_feature 1..998: note = Description of sequence: construct, name: eGFP-S-Mal-SulfoCy5
  source 1..998: mol_type = other DNA
  modified_base 998: mod_base = OTHER; note = deoxyadenosine-3'methoxypropanethiol-maleimide-3H-I ndolium, 1-[6-[(6-aminohexyl)amino]-6-oxohexyl]-2-[5-(1,3- dihydro-1,3,3-trimethyl-5-sulfo-2H-indol-2-ylidene)-1,3-pe ntadien-1-yl]-3,3-dimethyl-5-sulfo-[salt]
  misc_feature 1..997: note = RNA
  misc_feature 998: note = DNA
  misc_feature 1..998: note = molecule is DNA/RNA hybrid (every T represent uracil)
aggaaataagagagaaaagaagagtaagaagaaatataagagccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaagctgccttctgcggggcttgccttctggccatgcccttcttctctcccttgcacctgtacctcttggtctttgaataaagcctgagtaggaagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 86 - DNA, 998 bp, synthetic construct
  misc_feature 1..998: note = Description of sequence: construct, name: mCherry-S-Mal-SulfoCy5
  source 1..998: mol_type = other DNA
  modified_base 998: mod_base = OTHER; note = deoxyadenosine-3'methoxypropanethiol-maleimide-3H-I ndolium, 1-[6-[(6-aminohexyl)amino]-6-oxohexyl]-2-[5-(1,3- dihydro-1,3,3-trimethyl-5-sulfo-2H-indol-2-ylidene)-1,3-pe ntadien-1-yl]-3,3-dimethyl-5-sulfo-[salt]
  misc_feature 1..997: note = RNA
  misc_feature 998: note = DNA
  misc_feature 1..998: note = molecule is DNA/RNA hybrid (every T represent uracil)
aggaaataagagagaaaagaagagtaagaagaaatataagagccaccatggtgagcaagggcgaggaggacaacatggccatcatcaaggagttcatgcggttcaaggtgcacatggagggcagcgtgaacggccacgagttcgagatcgagggcgagggcgagggccggccctacgagggcacccagaccgccaagctgaaggtgaccaagggcggccccctgcccttcgcctgggacatcctgagcccccagttcatgtacggcagcaaggcctacgtgaagcaccccgccgacatccccgactacctgaagctgagcttccccgagggcttcaagtgggagcgggtgatgaacttcgaggacggcggcgtggtgaccgtgacccaggacagcagcctgcaggacggcgagttcatctacaaggtgaagctgcggggcaccaacttccccagcgacggccccgtgatgcagaagaagaccatgggctgggaggccagcagcgagcggatgtaccccgaggacggcgccctgaagggcgagatcaagcagcggctgaagctgaaggacggcggccactacgacgccgaggtgaagaccacctacaaggccaagaagcccgtgcagctgcccggcgcctacaacgtgaacatcaagctggacatcaccagccacaacgaggactacaccatcgtggagcagtacgagcgggccgagggccggcacagcaccggcggcatggacgagctgtacaagagcggcaactgagctgccttctgcggggcttgccttctggccatgcccttcttctctcccttgcacctgtacctcttggtctttgaataaagcctgagtaggaagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 87 - DNA, 1931 bp, synthetic construct
  misc_feature 1..1931: note = Description of sequence: construct, name: FLuc-S-Mal-SulfoCy5
  source 1..1931: mol_type = other DNA
  modified_base 1931: mod_base = OTHER; note = deoxyadenosine-3'methoxypropanethiol-maleimide-3H-I ndolium, 1-[6-[(6-aminohexyl)amino]-6-oxohexyl]-2-[5-(1,3- dihydro-1,3,3-trimethyl-5-sulfo-2H-indol-2-ylidene)-1,3-pe ntadien-1-yl]-3,3-dimethyl-5-sulfo-[salt]
  misc_feature 1..1930: note = RNA
  misc_feature 1931: note = DNA
  misc_feature 1..1931: note = molecule is DNA/RNA hybrid (every T represent uracil)
aggaaataagagagaaaagaagagtaagaagaaatataagagccaccatggaggacgccaagaacatcaagaagggccccgcccccttctaccccctggaggacggcaccgccggcgagcagctgcacaaggccatgaagcggtacgccctggtgcccggcaccatcgccttcaccgacgcccacatcgaggtggacatcacctacgccgagtacttcgagatgagcgtgcggctggccgaggccatgaagcggtacggcctgaacaccaaccaccggatcgtggtgtgcagcgagaacagcctgcagttcttcatgcccgtgctgggcgccctgttcatcggcgtggccgtggcccccgccaacgacatctacaacgagcgggagctgctgaacagcatgggcatcagccagcccaccgtggtgttcgtgagcaagaagggcctgcagaagatcctgaacgtgcagaagaagctgcccatcatccagaagatcatcatcatggacagcaagaccgactaccagggcttccagagcatgtacaccttcgtgaccagccacctgccccccggcttcaacgagtacgacttcgtgcccgagagcttcgaccgggacaagaccatcgccctgatcatgaacagcagcggcagcaccggcctgcccaagggcgtggccctgccccaccggaccgcctgcgtgcggttcagccacgcccgggaccccatcttcggcaaccagatcatccccgacaccgccatcctgagcgtggtgcccttccaccacggcttcggcatgttcaccaccctgggctacctgatctgcggcttccgggtggtgctgatgtaccggttcgaggaggagctgttcctgcggagcctgcaggactacaagatccagagcgccctgctggtgcccaccctgttcagcttcttcgccaagagcaccctgatcgacaagtacgacctgagcaacctgcacgagatcgccagcggcggcgcccccctgagcaaggaggtgggcgaggccgtggccaagcggttccacctgcccggcatccggcagggctacggcctgaccgagaccaccagcgccatcctgatcacccccgagggcgacgacaagcccggcgccgtgggcaaggtggtgcccttcttcgaggccaaggtggtggacctggacaccggcaagaccctgggcgtgaaccagcggggcgagctgtgcgtgcggggccccatgatcatgagcggctacgtgaacaaccccgaggccaccaacgccctgatcgacaaggacggctggctgcacagcggcgacatcgcctactgggacgaggacgagcacttcttcatcgtggaccggctgaagagcctgatcaagtacaagggctaccaggtggcccccgccgagctggagagcatcctgctgcagcaccccaacatcttcgacgccggcgtggccggcctgcccgacgacgacgccggcgagctgcccgccgccgtggtggtgctggagcacggcaagaccatgaccgagaaggagatcgtggactacgtggccagccaggtgaccaccgccaagaagctgcggggcggcgtggtgttcgtggacgaggtgcccaagggcctgaccggcaagctggacgcccggaagatccgggagatcctgatcaaggccaagaagggcggcaagatcgccgtgtgagctgccttctgcggggcttgccttctggccatgcccttcttctctcccttgcacctgtacctcttggtctttgaataaagcctgagtaggaagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 88 - DNA, 1931 bp, synthetic construct; identical to SEQ ID NO: 87
  misc_feature 1..1931: note = Description of sequence: construct, name: FLuc-S-Mal-GalNAc
  source 1..1931: mol_type = other DNA
  modified_base 1931: mod_base = OTHER; note = deoxyadenosine-3'methoxpropanethiol-maleimide-N-ace tylgalactosamine
  misc_feature 1..1930: note = RNA
  misc_feature 1931: note = DNA
  misc_feature 1..1931: note = molecule is DNA/RNA hybrid (every T represent uracil)

SEQ ID NO: 89 - DNA, 1930 bp, synthetic construct
  misc_feature 1..1930: note = Description of sequence: construct, name: FLuc-NC-S-Mal-GalNAc
  source 1..1930: mol_type = other DNA
  modified_base 1925: mod_base = OTHER; note = 2'-O-methoxy-ethyl adenosine
  misc_feature 1925^1926: note = 5'-3'-phosphorothioate linkage
  modified_base 1926: mod_base = OTHER; note = 2'-O-methoxy-ethyl adenosine
  misc_feature 1926^1927: note = 5'-3'-phosphorothioate linkage
  modified_base 1927: mod_base = OTHER; note = 2'-O-methoxy-ethyl adenosine
  misc_feature 1927^1928: note = 5'-3'-phosphorothioate linkage
  modified_base 1928: mod_base = OTHER; note = 2'-O-methoxy-ethyl adenosine
  misc_feature 1928^1929: note = 5'-3'-phosphorothioate linkage
  modified_base 1929: mod_base = OTHER; note = 2'-O-methoxy-ethyl adenosine
  misc_feature 1929^1930: note = 5'-3'-phosphorothioate linkage
  modified_base 1930: mod_base = OTHER; note = 2'-O-methoxy-ethyl adenosine
aggaaataagagagaaaagaagagtaagaagaaatataagagccaccatggaggacgccaagaacatcaagaagggccccgcccccttctaccccctggaggacggcaccgccggcgagcagctgcacaaggccatgaagcggtacgccctggtgcccggcaccatcgccttcaccgacgcccacatcgaggtggacatcacctacgccgagtacttcgagatgagcgtgcggctggccgaggccatgaagcggtacggcctgaacaccaaccaccggatcgtggtgtgcagcgagaacagcctgcagttcttcatgcccgtgctgggcgccctgttcatcggcgtggccgtggcccccgccaacgacatctacaacgagcgggagctgctgaacagcatgggcatcagccagcccaccgtggtgttcgtgagcaagaagggcctgcagaagatcctgaacgtgcagaagaagctgcccatcatccagaagatcatcatcatggacagcaagaccgactaccagggcttccagagcatgtacaccttcgtgaccagccacctgccccccggcttcaacgagtacgacttcgtgcccgagagcttcgaccgggacaagaccatcgccctgatcatgaacagcagcggcagcaccggcctgcccaagggcgtggccctgccccaccggaccgcctgcgtgcggttcagccacgcccgggaccccatcttcggcaaccagatcatccccgacaccgccatcctgagcgtggtgcccttccaccacggcttcggcatgttcaccaccctgggctacctgatctgcggcttccgggtggtgctgatgtaccggttcgaggaggagctgttcctgcggagcctgcaggactacaagatccagagcgccctgctggtgcccaccctgttcagcttcttcgccaagagcaccctgatcgacaagtacgacctgagcaacctgcacgagatcgccagcggcggcgcccccctgagcaaggaggtgggcgaggccgtggccaagcggttccacctgcccggcatccggcagggctacggcctgaccgagaccaccagcgccatcctgatcacccccgagggcgacgacaagcccggcgccgtgggcaaggtggtgcccttcttcgaggccaaggtggtggacctggacaccggcaagaccctgggcgtgaaccagcggggcgagctgtgcgtgcggggccccatgatcatgagcggctacgtgaacaaccccgaggccaccaacgccctgatcgacaaggacggctggctgcacagcggcgacatcgcctactgggacgaggacgagcacttcttcatcgtggaccggctgaagagcctgatcaagtacaagggctaccaggtggcccccgccgagctggagagcatcctgctgcagcaccccaacatcttcgacgccggcgtggccggcctgcccgacgacgacgccggcgagctgcccgccgccgtggtggtgctggagcacggcaagaccatgaccgagaaggagatcgtggactacgtggccagccaggtgaccaccgccaagaagctgcggggcggcgtggtgttcgtggacgaggtgcccaagggcctgaccggcaagctggacgcccggaagatccgggagatcctgatcaaggccaagaagggcggcaagatcgccgtgtgagctgccttctgcggggcttgccttctggccatgcccttcttctctcccttgcacctgtacctcttggtctttgaataaagcctgagtaggaagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 90 - DNA, 998 bp, synthetic construct; identical to SEQ ID NO: 85
  misc_feature 1..998: note = Description of sequence: construct, name: eGFP-S-Mal-P19-Mal
  source 1..998: mol_type = other DNA
  modified_base 998: mod_base = OTHER; note = deoxyadenosine-3'methoxypropanethiol-maleimide-nove mdec-ethylether-maleimide
  misc_feature 1..997: note = RNA
  misc_feature 998: note = DNA
  misc_feature 1..998: note = molecule is DNA/RNA hybrid (every T represent uracil)

SEQ ID NO: 91 - DNA, 998 bp, synthetic construct; identical to SEQ ID NO: 86
  misc_feature 1..998: note = Description of sequence: construct, name: mCherry-S-Mal-P19-Mal
  source 1..998: mol_type = other DNA
  modified_base 998: mod_base = OTHER; note = deoxyadenosine-3'methoxypropanethiol-maleimide-nove mdec-ethylether-maleimide
  misc_feature 1..997: note = RNA
  misc_feature 998: note = DNA
  misc_feature 1..998: note = molecule is DNA/RNA hybrid (every T represent uracil)

SEQ ID NO: 92 - DNA, 1931 bp, synthetic construct; identical to SEQ ID NO: 87
  misc_feature 1..1931: note = Description of sequence: construct, name:FLuc-S-Mal-P19-Mal
  source 1..1931: mol_type = other DNA
  modified_base 1931: mod_base = OTHER; note = deoxyadenosine-3'methoxypropanethiol-maleimide-nove mdec-ethylether-maleimide
  misc_feature 1..1930: note = RNA
  misc_feature 1931: note = DNA
  misc_feature 1..1931: note = molecule is DNA/RNA hybrid (every T represent uracil)

SEQ ID NO: 93 - DNA, 998 bp, synthetic construct; identical to SEQ ID NO: 85
  misc_feature 1..998: note = Description of sequence: construct, name: eGFP-S-Mal-P19-Mal-S-PolyA
  source 1..998: mol_type = other DNA
  modified_base 998: mod_base = OTHER; note = deoxyadenosine-3'methoxpropanethiol-maleimide-novem dec-ethylether-maleimide-polyA-linkage
  misc_feature 1..997: note = RNA
  misc_feature 998: note = DNA
  misc_feature 1..998: note = molecule is DNA/RNA hybrid (every T represent uracil)

SEQ ID NO: 94 - DNA, 998 bp, synthetic construct; identical to SEQ ID NO: 85
  misc_feature 1..998: note = Description of sequence: construct, name: eGFP-S-Mal-P19-Mal-S-eGFP
  source 1..998: mol_type = other DNA
  modified_base 998: mod_base = OTHER; note = deoxyadenosine-3'methoxpropanethiol-maleimide-novem dec-ethylether-maleimide-polyA-linkage
  misc_feature 1..997: note = RNA
  misc_feature 998: note = DNA
  misc_feature 1..998: note = molecule is DNA/RNA hybrid (every T represent uracil)

SEQ ID NO: 95 - DNA, 998 bp, synthetic construct; identical to SEQ ID NO: 85
  misc_feature 1..998: note = Description of sequence: construct, name: eGFP-SH
  source 1..998: mol_type = other DNA
  modified_base 998: mod_base = OTHER; note = deoxyadenosine-1-O-dimethoxytrityl-propyl-disulfide
  misc_feature 1..997: note = RNA
  misc_feature 998: note = DNA
  misc_feature 1..998: note = molecule is DNA/RNA hybrid (every T represent uracil)

SEQ ID NO: 96 - DNA, 998 bp, synthetic construct; identical to SEQ ID NO: 85
  misc_feature 1..998: note = Description of sequence: construct, name: eGFP-S-Mal-P19-Mal-S-mCherry
  source 1..998: mol_type = other DNA
  modified_base 998: mod_base = OTHER; note = deoxyadenosine-3'methoxpropanethiol-maleimide-novem dec-ethylether-maleimide-polyA-linkage
  misc_feature 1..997: note = RNA
  misc_feature 998: note = DNA
  misc_feature 1..998: note = molecule is DNA/RNA hybrid (every T represent uracil)

SEQ ID NO: 97 - DNA, 998 bp, synthetic construct; identical to SEQ ID NO: 86
  misc_feature 1..998: note = Description of sequence: construct, name: mCherry-SH
  source 1..998: mol_type = other DNA
  modified_base 998: mod_base = OTHER; note = deoxyadenosine-1-O-dimethoxytrityl-propyl-disulfide
  misc_feature 1..997: note = RNA
  misc_feature 998: note = DNA
  misc_feature 1..998: note = molecule is DNA/RNA hybrid (every T represent uracil)

SEQ ID NO: 98 - DNA, 998 bp, synthetic construct; identical to SEQ ID NO: 85
  misc_feature 1..998: note = Description of sequence: construct, name: eGFP-S-Mal-SulfoCy5
  source 1..998: mol_type = other DNA
  modified_base 998: mod_base = OTHER; note = deoxyadenosine-3'methoxypropanethiol-maleimide-3H-I ndolium, 1-[6-[(6-aminohexyl)amino]-6-oxohexyl]-2-[5-(1,3- dihydro-1,3,3-trimethyl-5-sulfo-2H-indol-2-ylidene)-1,3-pe ntadien-1-yl]-3,3-dimethyl-5-sulfo-[salt]
  misc_feature 1..997: note = RNA
  misc_feature 998: note = DNA
  misc_feature 1..998: note = molecule is DNA/RNA hybrid (every T represent uracil)

SEQ ID NO: 99 - DNA, 982 bp, synthetic construct
  misc_feature 1..982: note = Description of sequence: construct, name: eGFP-Cyt-S-Mal-P19-Mal
  source 1..982: mol_type = other DNA
  modified_base 982: mod_base = OTHER; note = deoxycytidine-3'methoxpropanethiol-maleimide-novemd ec-ethylether-maleimide
  misc_feature 1..981: note = RNA
  misc_feature 982: note = DNA
  misc_feature 1..982: note = molecule is DNA/RNA hybrid (every T represent uracil)
aggaaataagagagaaaagaagagtaagaagaaatataagagccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaagctgccttctgcggggcttgccttctggccatgcccttcttctctcccttgcacctgtacctcttggtctttgaataaagcctgagtaggaagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaac

SEQ ID NO: 100 - DNA, 982 bp, synthetic construct; identical to SEQ ID NO: 99
  misc_feature 1..982: note = Description of sequence: construct, name: eGFP-S-Mal-P19-Mal-Peptide
  source 1..982: mol_type = other DNA
  modified_base 982: mod_base = OTHER; note = deoxycytidine-3'methoxpropanethiol-maleimide-novemd ec-ethylether-maleimide-peptide
  misc_feature 1..981: note = RNA
  misc_feature 982: note = DNA
  misc_feature 1..982: note = molecule is DNA/RNA hybrid (every T represent uracil)

SEQ ID NO: 101 - protein, 16 aa, synthetic construct
  REGION 1..16: note = Description of sequence: Artificial sequence, name: Peptide 1
PASPASPASPASPASC

SEQ ID NO: 102 - protein, 46 aa, synthetic construct
  REGION 1..46: note = Description of sequence: Artificial sequence, name: Peptide 2
PASPASPASPASPASPASPASPASPASPASPASPASPASPASPASC

SEQ ID NO: 103 - protein, 20 aa, synthetic construct
  REGION 1..20: note = Description of sequence: Artificial sequence, name: Peptide 3
CPASPASPASPASPASKDEL

SEQ ID NO: 104 - protein, 22 aa, synthetic construct
  REGION 1..22: note = Description of sequence: Artificial sequence, name: Peptide 4
CPASPASPASPASPASDEKKMP

SEQ ID NO: 105 - protein, 21 aa, synthetic construct
  REGION 1..21: note = Description of sequence: Artificial sequence, name: Peptide 5
RGVPHIVMVDAYKRYKSGGSC

SEQ ID NO: 106 - protein, 113 aa, synthetic construct
  REGION 1..113: note = Description of sequence: Artificial sequence, name: Peptide 6
VTTLSGLSGEQGPSGDMTTEEDSATHIKFSKRDEDGRELAGATMELRDCSGKTISTWISDGHVKDFYLYPGKYTFVETAAPDGYEVATPIEFTVNEDGQVTVDGEATEGDAHT

SEQ ID NO: 107 - protein, 40 aa, synthetic construct
  REGION 1..40: note = Description of sequence: Artificial sequence, name: Peptide 7
  SITE 2: note = Aib
  SITE 8: note = Aib
  SITE 21: note = branched peptide side chain: Glycine-Glycine-Cysteine
YAEGTFTSDYSIXLDKIAQKXAFVQWLIAGGPSSGAPPPS

SEQ ID NO: 108 - RNA, 1576 nt, synthetic construct
  misc_feature 1..1576: note = Description of sequence: Artificial sequence, name: Adalimumab HC Nucleic Acid
  source 1..1576: mol_type = other RNA
aggaaataagagagaaaagaagagtaagaagaaatataagagccaccatggaagtgcagctggtggaaagcggcggcggcctggtgcagccgggccgcagcctgcgcctgagctgcgcggcgagcggctttacctttgatgattatgcgatgcattgggtgcgccaggcgccgggcaaaggcctggaatgggtgagcgcgattacctggaacagcggccatattgattatgcggatagcgtggaaggccgctttaccattagccgcgataacgcgaaaaacagcctgtatctgcagatgaacagcctgcgcgcggaagataccgcggtgtattattgcgcgaaagtgagctatctgagcaccgcgagcagcctggattattggggccagggcaccctggtgaccgtgagcagcgcgagcaccaaaggcccgagcgtgtttccgctggcgccgagcagcaaaagcaccagcggcggcaccgcggcgctgggctgcctggtgaaagattattttccggaaccggtgaccgtgagctggaacagcggcgcgctgaccagcggcgtgcatacctttccggcggtgctgcagagcagcggcctgtatagcctgagcagcgtggtgaccgtgccgagcagcagcctgggcacccagacctatatttgcaacgtgaaccataaaccgagcaacaccaaagtggataaaaaagtggaaccgaaaagctgcgataaaacccatacctgcccgccgtgcccggcgccggaactgctgggcggcccgagcgtgtttctgtttccgccgaaaccgaaagataccctgatgattagccgcaccccggaagtgacctgcgtggtggtggatgtgagccatgaagatccggaagtgaaatttaactggtatgtggatggcgtggaagtgcataacgcgaaaaccaaaccgcgcgaagaacagtataacagcacctatcgcgtggtgagcgtgctgaccgtgctgcatcaggattggctgaacggcaaagaatataaatgcaaagtgagcaacaaagcgctgccggcgccgattgaaaaaaccattagcaaagcgaaaggccagccgcgcgaaccgcaggtgtataccctgccgccgagccgcgatgaactgaccaaaaaccaggtgagcctgacctgcctggtgaaaggcttttatccgagcgatattgcggtggaatgggaaagcaacggccagccggaaaacaactataaaaccaccccgccggtgctggatagcgatggcagcttttttctgtatagcaaactgaccgtggataaaagccgctggcagcagggcaacgtgtttagctgcagcgtgatgcatgaagcgctgcataaccattatacccagaaaagcctgagcctgagcccgggcaaagctgccttctgcggggcttgccttctggccatgcccttcttctctcccttgcacctgtacctcttggtctttgaataaagcctgagtaggaagaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 109 - protein, 451 aa, synthetic construct
  REGION 1..451: note = Description of sequence: Artificial sequence, name: Adalimumab HC Amino Acid
EVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 110 - RNA, 865 nt, synthetic construct
  misc_feature 1..865: note = Description of sequence: Artificial sequence, name: Adalimumab LC Nucleic Acid
  source 1..865: mol_type = other RNA
aggaaataagagagaaaagaagagtaagaagaaatataagagccaccatggatattcagatgacccagagcccgagcagcctgagcgcgagcgtgggcgatcgcgtgaccattacctgccgcgcgagccagggcattcgcaactatctggcgtggtatcagcagaaaccgggcaaagcgccgaaactgctgatttatgcggcgagcaccctgcagagcggcgtgccgagccgctttagcggcagcggcagcggcaccgattttaccctgaccattagcagcctgcagccggaagatgtggcgacctattattgccagcgctataaccgcgcgccgtatacctttggccagggcaccaaagtggaaattaaacgcaccgtggcggcgccgagcgtgtttatttttccgccgagcgatgaacagctgaaaagcggcaccgcgagcgtggtgtgcctgctgaacaacttttatccgcgcgaagcgaaagtgcagtggaaagtggataacgcgctgcagagcggcaacagccaggaaagcgtgaccgaacaggatagcaaagatagcacctatagcctgagcagcaccctgaccctgagcaaagcggattatgaaaaacataaagtgtatgcgtgcgaagtgacccatcagggcctgagcagcccggtgaccaaaagctttaaccgcggcgaatgcgctgccttctgcggggcttgccttctggccatgcccttcttctctcccttgcacctgtacctcttggtctttgaataaagcctgagtaggaagaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 111 - protein, 214 aa, synthetic construct
  REGION 1..214: note = Description of sequence: Artificial sequence, name: Adalimumab LC Amino Acid
DIQMTQSPSSLSASVGDRVTITCRASQGIRNYLAWYQQKPGKAPKLLIYAASTLQSGVPSRFSGSGSGTDFTLTISSLQPEDVATYYCQRYNRAPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 112 - RNA, 1576 nt, synthetic construct
  misc_feature 1..1576: note = Description of sequence: Artificial sequence, name: Duplimab HC Nucleic Acid
  source 1..1576: mol_type = other RNA
aggaaataagagagaaaagaagagtaagaagaaatataagagccaccatggaagtgcagctggtggaaagcggcggcggcctggaacagccgggcggcagcctgcgcctgagctgcgcgggcagcggctttacctttcgcgattatgcgatgacctgggtgcgccaggcgccgggcaaaggcctggaatgggtgagcagcattagcggcagcggcggcaacacctattatgcggatagcgtgaaaggccgctttaccattagccgcgataacagcaaaaacaccctgtatctgcagatgaacagcctgcgcgcggaagataccgcggtgtattattgcgcgaaagatcgcctgagcattaccattcgcccgcgctattatggcctggatgtgtggggccagggcaccaccgtgaccgtgagcagcgcgagcaccaaaggcccgagcgtgtttccgctggcgccgtgcagccgcagcaccagcgaaagcaccgcggcgctgggctgcctggtgaaagattattttccggaaccggtgaccgtgagctggaacagcggcgcgctgaccagcggcgtgcatacctttccggcggtgctgcagagcagcggcctgtatagcctgagcagcgtggtgaccgtgccgagcagcagcctgggcaccaaaacctatacctgcaacgtggatcataaaccgagcaacaccaaagtggataaacgcgtggaaagcaaatatggcccgccgtgcccgccgtgcccggcgccggaatttctgggcggcccgagcgtgtttctgtttccgccgaaaccgaaagataccctgatgattagccgcaccccggaagtgacctgcgtggtggtggatgtgagccaggaagatccggaagtgcagtttaactggtatgtggatggcgtggaagtgcataacgcgaaaaccaaaccgcgcgaagaacagtttaacagcacctatcgcgtggtgagcgtgctgaccgtgctgcatcaggattggctgaacggcaaagaatataaatgcaaagtgagcaacaaaggcctgccgagcagcattgaaaaaaccattagcaaagcgaaaggccagccgcgcgaaccgcaggtgtataccctgccgccgagccaggaagaaatgaccaaaaaccaggtgagcctgacctgcctggtgaaaggcttttatccgagcgatattgcggtggaatgggaaagcaacggccagccggaaaacaactataaaaccaccccgccggtgctggatagcgatggcagcttttttctgtatagccgcctgaccgtggataaaagccgctggcaggaaggcaacgtgtttagctgcagcgtgatgcatgaagcgctgcataaccattatacccagaaaagcctgagcctgagcctgggcgctgccttctgcggggcttgccttctggccatgcccttcttctctcccttgcacctgtacctcttggtctttgaataaagcctgagtaggaagaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 113 - protein, 451 aa, synthetic construct
  REGION 1..451: note = Description of sequence: Artificial sequence, name: Duplimab HC Amino Acid
EVQLVESGGGLEQPGGSLRLSCAGSGFTFRDYAMTWVRQAPGKGLEWVSSISGSGGNTYYADSVKGRFTISRDNSKNTLYLQMNSLRAEDTAVYYCAKDRLSITIRPRYYGLDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 114 - RNA, 880 nt, synthetic construct
  misc_feature 1..880: note = Description of sequence: Artificial sequence, name: Duplimab LC Nucleic Acid
  source 1..880: mol_type = other RNA
aggaaataagagagaaaagaagagtaagaagaaatataagagccaccatggatattgtgatgacccagagcccgctgagcctgccggtgaccccgggcgaaccggcgagcattagctgccgcagcagccagagcctgctgtatagcattggctataactatctggattggtatctgcagaaaagcggccagagcccgcagctgctgatttatctgggcagcaaccgcgcgagcggcgtgccggatcgctttagcggcagcggcagcggcaccgattttaccctgaaaattagccgcgtggaagcggaagatgtgggcttttattattgcatgcaggcgctgcagaccccgtatacctttggccagggcaccaaactggaaattaaacgcaccgtggcggcgccgagcgtgtttatttttccgccgagcgatgaacagctgaaaagcggcaccgcgagcgtggtgtgcctgctgaacaacttttatccgcgcgaagcgaaagtgcagtggaaagtggataacgcgctgcagagcggcaacagccaggaaagcgtgaccgaacaggatagcaaagatagcacctatagcctgagcagcaccctgaccctgagcaaagcggattatgaaaaacataaagtgtatgcgtgcgaagtgacccatcagggcctgagcagcccggtgaccaaaagctttaaccgcggcgaatgcgctgccttctgcggggcttgccttctggccatgcccttcttctctcccttgcacctgtacctcttggtctttgaataaagcctgagtaggaagaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 115 - protein, 219 aa, synthetic construct
  REGION 1..219: note = Description of sequence: Artificial sequence, name: Duplimab LC Amino Acid
DIVMTQSPLSLPVTPGEPASISCRSSQSLLYSIGYNYLDWYLQKSGQSPQLLIYLGSNRASGVPDRFSGSGSGTDFTLKISRVEAEDVGFYYCMQALQTPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 116 - RNA, 1570 nt, synthetic construct
  misc_feature 1..1570: note = Description of sequence: Artificial sequence, name: Trastuzumab HC Nucleic Acid
  source 1..1570: mol_type = other RNA
aggaaataagagagaaaagaagagtaagaagaaatataagagccaccatggaagtgcagctggtggaaagcggcggcggcctggtgcagccgggcggcagcctgcgcctgagctgcgcggcgagcggctttaacattaaagatacctatattcattgggtgcgccaggcgccgggcaaaggcctggaatgggtggcgcgcatttatccgaccaacggctatacccgctatgcggatagcgtgaaaggccgctttaccattagcgcggataccagcaaaaacaccgcgtatctgcagatgaacagcctgcgcgcggaagataccgcggtgtattattgcagccgctggggcggcgatggcttttatgcgatggattattggggccagggcaccctggtgaccgtgagcagcgcgagcaccaaaggcccgagcgtgtttccgctggcgccgagcagcaaaagcaccagcggcggcaccgcggcgctgggctgcctggtgaaagattattttccggaaccggtgaccgtgagctggaacagcggcgcgctgaccagcggcgtgcatacctttccggcggtgctgcagagcagcggcctgtatagcctgagcagcgtggtgaccgtgccgagcagcagcctgggcacccagacctatatttgcaacgtgaaccataaaccgagcaacaccaaagtggataaaaaagtggaaccgaaaagctgcgataaaacccatacctgcccgccgtgcccggcgccggaactgctgggcggcccgagcgtgtttctgtttccgccgaaaccgaaagataccctgatgattagccgcaccccggaagtgacctgcgtggtggtggatgtgagccatgaagatccggaagtgaaatttaactggtatgtggatggcgtggaagtgcataacgcgaaaaccaaaccgcgcgaagaacagtataacagcacctatcgcgtggtgagcgtgctgaccgtgctgcatcaggattggctgaacggcaaagaatataaatgcaaagtgagcaacaaagcgctgccggcgccgattgaaaaaaccattagcaaagcgaaaggccagccgcgcgaaccgcaggtgtataccctgccgccgagccgcgaagaaatgaccaaaaaccaggtgagcctgacctgcctggtgaaaggcttttatccgagcgatattgcggtggaatgggaaagcaacggccagccggaaaacaactataaaaccaccccgccggtgctggatagcgatggcagcttttttctgtatagcaaactgaccgtggataaaagccgctggcagcagggcaacgtgtttagctgcagcgtgatgcatgaagcgctgcataaccattatacccagaaaagcctgagcctgagcccgggcgctgccttctgcggggcttgccttctggccatgcccttcttctctcccttgcacctgtacctcttggtctttgaataaagcctgagtaggaagaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 117 - protein, 448 aa, synthetic construct
  REGION 1..448: note = Description of sequence: Artificial sequence, name: Trastuzumab HC Amino Acid
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 118 - RNA, 865 nt, synthetic construct
  misc_feature 1..865: note = Description of sequence: Artificial sequence, name: Trastuzumab LC Nucleic Acid
  source 1..865: mol_type = other RNA
aggaaataagagagaaaagaagagtaagaagaaatataagagccaccatggatattcagatgacccagagcccgagcagcctgagcgcgagcgtgggcgatcgcgtgaccattacctgccgcgcgagccaggatgtgaacaccgcggtggcgtggtatcagcagaaaccgggcaaagcgccgaaactgctgatttatagcgcgagctttctgtatagcggcgtgccgagccgctttagcggcagccgcagcggcaccgattttaccctgaccattagcagcctgcagccggaagattttgcgacctattattgccagcagcattataccaccccgccgacctttggccagggcaccaaagtggaaattaaacgcaccgtggcggcgccgagcgtgtttatttttccgccgagcgatgaacagctgaaaagcggcaccgcgagcgtggtgtgcctgctgaacaacttttatccgcgcgaagcgaaagtgcagtggaaagtggataacgcgctgcagagcggcaacagccaggaaagcgtgaccgaacaggatagcaaagatagcacctatagcctgagcagcaccctgaccctgagcaaagcggattatgaaaaacataaagtgtatgcgtgcgaagtgacccatcagggcctgagcagcccggtgaccaaaagctttaaccgcggcgaatgcgctgccttctgcggggcttgccttctggccatgcccttcttctctcccttgcacctgtacctcttggtctttgaataaagcctgagtaggaagaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 119 - protein, 213 aa, synthetic construct
  REGION 1..213: note = Description of sequence: Artificial sequence, name: Trastuzumab LC Amino Acid
DIQMTQSPSSLSASVGDRVTITCRASQDVNAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 120 - RNA, 340 nt, synthetic construct
  misc_feature 1..340: note = Description of sequence: Artificial sequence, name: Exenatide Nucleic Acid
  source 1..340: mol_type = other RNA
aggaaataagagagaaaagaagagtaagaagaaatataagagccaccatgcatggcgaaggcacctttaccagcgatctgagcaaacagatggaagaagaagcggtgcgcctgtttattgaatggctgaaaaacggcggcccgagcagcggcgcgccgccgccgagcgctgccttctgcggggcttgccttctggccatgcccttcttctctcccttgcacctgtacctcttggtctttgaataaagcctgagtaggaagaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 121 - protein, 39 aa, synthetic construct
  REGION 1..39: note = Description of sequence: Artificial sequence, name: Exenatide Amino Acid
HGEGTFTSDLSKQMEEEAVRLFIEWLKNGGPSSGAPPPS

SEQ ID NO: 122 - RNA, 316 nt, synthetic construct
  misc_feature 1..316: note = Description of sequence: Artificial sequence, name: GLP-1 Nucleic Acid
  source 1..316: mol_type = other RNA
aggaaataagagagaaaagaagagtaagaagaaatataagagccaccatgcatgcggaaggcacctttaccagcgatgtgagcagctatctggaaggccaggcggcgaaagaatttattgcgtggctggtgaaaggccgcggcgctgccttctgcggggcttgccttctggccatgcccttcttctctcccttgcacctgtacctcttggtctttgaataaagcctgagtaggaagaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 123 - protein, 31 aa, synthetic construct
  REGION 1..31: note = Description of sequence: Artificial sequence, name: GLP-1 Amino Acid
HAEGTFTSDVSSYLEGQAAKEFIAWLVKGRG

SEQ ID NO: 124 - RNA, 349 nt, synthetic construct
  misc_feature 1..349: note = Description of sequence: Artificial sequence, name: Gastric Inhibitory Peptide Nucleic Acid
  source 1..349: mol_type = other RNA
aggaaataagagagaaaagaagagtaagaagaaatataagagccaccatgtatgcggaaggcacctttattagcgattatagcattgcgatggataaaattcatcagcaggattttgtgaactggctgctggcgcagaaaggcaaaaaaaacgattggaaacataacattacccaggctgccttctgcggggcttgccttctggccatgcccttcttctctcccttgcacctgtacctcttggtctttgaataaagcctgagtaggaagaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 125 - protein, 42 aa, synthetic construct
  REGION 1..42: note = Description of sequence: Artificial sequence, name: Gastric Inhibitory Peptide Amino Acid
YAEGTFISDYSIAMDKIHQQDFVNWLLAQKGKKNDWKHNITQ

SEQ ID NO: 126 - RNA, 325 nt, synthetic construct
  misc_feature 1..325: note = Description of sequence: Artificial sequence, name: Tariparatide Nucleic Acid
  source 1..325: mol_type = other RNA
aggaaataagagagaaaagaagagtaagaagaaatataagagccaccatgagcgtgagcgaaattcagctgatgcataacctgggcaaacatctgaacagcatggaacgcgtggaatggctgcgcaaaaaactgcaggatgtgcataactttgctgccttctgcggggcttgccttctggccatgcccttcttctctcccttgcacctgtacctcttggtctttgaataaagcctgagtaggaagaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 127 - protein, 34 aa, synthetic construct
  REGION 1..34: note = Description of sequence: Artificial sequence, name: Tariparatide Amino Acid
SVSEIQLMHNLGKHLNSMERVEWLRKKLQDVHNF

SEQ ID NO: 128 - RNA, 1615 nt, synthetic construct
  misc_feature 1..1615: note = Description of sequence: Artificial sequence, name: Pembrolizumab Heavy Chain (PembrolizumabHC)
  source 1..1615: mol_type = other RNA
aggaaataagagagaaaagaagagtaagaagaaatataagagccaccatgagggcttggatcttctttctgctctgcctggccgggcgcgccttggcccaagtgcagctggtgcagagcggtgtagaggtcaagaaacccggcgctagcgtgaaggtgagctgcaaggctagcggctacaccttcaccaactactacatgtactgggtgagacaagcccccggccaaggcctggagtggatgggcggcatcaaccctagcaacggcggcaccaacttcaacgagaagttcaagaacagagtgaccctgaccaccgacagcagcaccaccaccgcctacatggagctgaaaagcctgcagttcgacgacaccgccgtgtactactgcgctagaagagactacagattcgacatgggcttcgactactggggccaaggcaccaccgtgaccgtgagcagcgctagcaccaagggtcctagcgttttccccctagccccctgcagcagaagcacaagcgagagcaccgccgccctgggctgccttgtcaaggactatttccccgagcccgtgaccgtatcctggaacagcggcgcacttacgtccggcgtgcacaccttccccgccgtgctgcagagcagcggcctgtacagcctgagcagcgtggtgaccgtgccttcgagcagcctgggcaccaagacctacacctgcaacgtggaccacaagcctagcaacaccaaggtggacaagagagttgagtccaagtatggacccccgtgtccaccctgtcccgccccggagttcctgggcggccctagcgtattcctatttccccccaagcccaaggacaccctgatgatcagcagaacccccgaggtgacctgcgtggtggtggacgtgagccaagaggaccccgaggtgcagttcaactggtacgtggacggggtggaagttcacaacgccaagaccaagcctagagaggagcagttcaacagcacctacagagtggtgagcgtgctgaccgtgctgcaccaagactggctgaacggcaaggagtacaagtgcaaggtgagcaacaagggcctgcctagcagcatcgagaagaccatcagcaaggccaaggggcagcctagagagccccaagtgtacaccctgccccctagccaagaggagatgaccaagaaccaagtgagcctgacctgccttgtaaagggcttctaccctagcgacatcgccgtggagtgggagagcaacgggcagcccgagaacaactacaagaccaccccccccgtgctggacagcgacggcagcttcttcctgtacagcagactgaccgtggacaagagcagatggcaagagggcaacgtgttcagctgcagcgtgatgcacgaggccctgcacaaccactacacacagaaaagcctgagcctgagcctgggcaagtaagctgccttctgcggggcttgccttctggccatgcccttcttctctcccttgcacctgtacctcttggtctttgaataaagcctgagtaggaagaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 129 - RNA, 928 nt, synthetic construct
  misc_feature 1..928: note = Description of sequence: Artificial sequence, name: Pembrolizumab Light Chain (PembrolizumabLC)
  source 1..928: mol_type = other RNA
aggaaataagagagaaaagaagagtaagaagaaatataagagccaccatgagggcttggatcttctttctgctctgcctggccgggcgcgccttggccgagatcgtactgacacagagccccgcgaccctgagcctgtcccccggtgagagagctaccctgagctgcagagctagcaagggcgtgagcacaagcggctacagctacctgcactggtatcagcagaagcccggccaagcccctagactgctgatctacctggctagctacctggagagcggcgtgcccgctagattcagcggcagcggcagcggcaccgacttcaccctgaccatcagcagcctggagcccgaggacttcgccgtgtactactgtcagcacagcagagacctgcccctgaccttcggcggcggcaccaaggtggagatcaagagaaccgtggccgcccctagcgtgttcatcttcccccctagcgacgagcagctgaaaagcggcaccgctagcgtggtgtgcctgctgaacaacttctaccctagagaggccaaggtgcagtggaaggtggacaacgccctgcagagcggcaacagccaagagagcgtgaccgagcaagacagcaaggacagcacctacagcctgagcagcaccctgaccctgagcaaggccgactacgagaagcacaaggtgtacgcctgcgaggtgacccaccaaggcctgagcagccccgtgaccaagagcttcaacagaggcgagtgctaagctgccttctgcggggcttgccttctggccatgcccttcttctctcccttgcacctgtacctcttggtctttgaataaagcctgagtaggaagaaaaaaaaaaaaaaaaaaaaaaaaaaaaaaaaaaaaaaaaaaaaaaaaaaaaaaaaaaaaaaaaaaaaaaaaaaaaaaaa